SEQUENCE LISTING (28 sequences)

SEQ ID NO: 1 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 2 - protein, 220 aa, Homo sapiens
MPLTGVGPGSCRRQIIRALCLLLLLLHAGSAKNIWKRALPARLAEKSRAEEAGAPGGPRQPRADRCPPPPRTLPPGACQAARCQADSECPRHRRCCYNGCAYACLEAVPPPPVLDWLVQPKPRWLGGNGWLLDGPEEVLQAEACSTTEDGAEPLLCPSGYECHILSPGDVAEGIPNRGQCVKQRRQADGRILRHKLYKEYPEGDSKNVAEPGRGQQKHFQ

SEQ ID NO: 3 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 4 - protein, 325 aa, Homo sapiens
MLPRVGCPALPLPPPPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 5 - protein, 639 aa, Homo sapiens
MEDSQDLNEQSVKKTCTESDVSQSQNSRSMEMQDLASPHTLVGGGDTPGSSKLEKSNLSSTSVTTNGTGGENMTVLNTADWLLSCNTPSSATMSLLAVKTEPLNSSETTATTGDGALDTFTGSVITSSGYSPRSAHQYSPQLYPSKPYPHILSTPAAQTMSAYAGQTQYSGMQQPAVYTAYSQTGQPYSLPTYDLGVMLPAIKTESGLSQTQSPLQSGCLSYSPGFSTPQPGQTPYSYQMPGSSFAPSSTIYANNSVSNSTNFSGSQQDYPSYTAFGQNQYAQYYSASTYGAYMTSNNTADGTPSSTSTYQLQESLPGLTNQPGEFDTMQSPSTPIKDLDERTCRSSGSKSRGRGRKNNPSPPPDSDLERVFVWDLDETIIVFHSLLTGSYAQKYGKDPPMAVTLGLRMEEMIFNLADTHLFFNDLEECDQVHIDDVSSDDNGQDLSTYSFATDGFHAAASSANLCLPTGVRGGVDWMRKLAFRYRRVKELYNTYKNNVGGLLGPAKRDAWLQLRAEIEGLTDSWLTNALKSLSIISTRSNCINVLVTTTQLIPALAKVLLYSLGGAFPIENIYSATKIGKESCFERIMQRFGRKVVYVVIGDGVEEEQAAKKHNMPFWRISSHSDLLALHQALELEYL

SEQ ID NO: 6 - protein, 236 aa, Homo sapiens
MHTTQKDTTYTKIFVGGLPYHTTDASLRKYFEVFGEIEEAVVITDRQTGKSRGYGFVTMADRAAAERACKDPNPIIDGRKANVNLAYLGAKPRIMQPGFAFGVQQLHPALIQRPFGIPAHYVYPQAFVQPGVVIPHVQPTAAAASTTPYIDYTGAAYAQYSAAAAAAAAAAAYDQYPYAASPAAAGYVTAGGYGYAVQQPITAAAPGTAAAAAAAAAAAAAFGQYQPQQLQTDRMQ

SEQ ID NO: 7 - DNA, 14193 bp, Homo sapiens
  source 1..14193: mol_type = other DNA
ctgcctcagcctcccgagtagctgggactacaggcgcgcgcgaccacgcccagctaatttttgtatttttagtagagatggggtttcaccatgttggccaggatggtctcgatttcttgacctcgtgatccgcccgcctcggcctcccagagtgctgggattacaggcataaaccaccgcgcccagcctctttcctcccctctctactcccggctccaagatcagccctggcagcagcagtgagggtcccaggcatcgccgggatgggatggaacaaggctccccttctccagggctacccctgggatgctggcagccccgtgtggcagggaagaggaactgcaggagcgtccttcagggaggagaggggccagaagacctcaacatggcgcccccaggtttcccacaggccccctcctgtagaccagccggccttcccctcagtgacgactcaaagtcagatttgaagagctctctggggacattcctctccctggatggtggtattctggggagcggatgtgaacttccctgaaggggagaggtgtaaactcccagcatcctgctaccaccctccgcccccatcacccttcattccccaggtgcaggctgggggcagaggagagggaaggggattaggtgctggggccggggaaccgagacccagggtgtcaccgctgacagcagcggggcccgcagagctgcagggagacagagccgcctccccgcggcgccgacgctgccatctagctagagggccccactctttctttaaccagaggtcacagcctcattgcctacccaggaggcagccccgagacgccccctgcggcctgggttttctccaccctgtccccgcgcccaccccagctctgccatccccggctgcaccgctttgtaaaggggacggctggtcagaccgaatcactgcgcgggctcgcgctgggtcgggcctggagactggatctggggcttccggagagccccaggctccccgcgcgcccatccacccgcagccgcctgagcatttatggtaatgtttgggttggggcgagcggccggatccccgccccctgcctccgccgggcgggacctcgaggccggctcctggggagcttagcgccggcgccttcccgccgcggccgggtcccctgggtggaggccctgggtgccggaggcctgaggtccggcaggtgggctggcccccgtgcggaggctcctaagaccggggggtacgagagggacagtcccagtcggtcggaggaggggtccgcggggttggggactccgatcccgctcaacctaccctaaccacccactgctgcccacccaacccccgcctctggcttaagagtccatcatccccaggaggagtcccgatgcatccacatcctctttctccttctctccccccaccccctggcagggaaggggctgagggaagaggccagcagctctcaggggagtcgtcggaatgctgggagcctgggaacagaccctgcggtccctgggaaagccaggctgtgggaatcccaccgtcagccccctccccaggatgggccaccggatgtcagagaccccagttccccaacggggatctttgtgagggtgggccctgccccccagttcaagccccagcctggctcgaccccagagttcagtaggccatctgtgacgaagacagcccctcccccgctctccgccctccacccccctgcctgctccaacataatcctcgccattgtgcccgtcgccccctccccccgctccccctctctagacccctgtacaatggggcctttgcagcctccagaatggagatgaactcacggtttttccaagtttaaaaatagcccctcccatcccgcagaaggccaccctccctctctcccatggtggtgtggggtgcacctccatcccggctgtgcctccttcctcctgccctgggtcaggtgcgaaggacatgggtgagacagcatccggcgggagcagggctgcttcccccgattcaatctcttcccccacatcttccatccaccccccagtacagtatccggtttcctgccccctacccttttccaccctgttttcactccttcagtataatcttctcctactcacacccacattctggaaaaattctccaacttccgcaccccctccaaagacccaggccggatcctattatctggcctggccagggggttccccagtctcggtaactgccaagattgttgggactcccaccccatgtcctcacagcagctgcaaacacctctttatcagcttccttcctcgagctacttctgacccacccttctcatatcctcacatcctggtcaaggcccctggatccagaagggtgggtcagaagtcgctaagttccctttgctggagctgagagaggacacttgggttccagtcttgccccttcatggcctctggggacaggtccatccttgtctttttacctccaactccccagggttccacttctccttacaatgagctgaaagctccacccatccaggagactgtggctgaggagcccaaccaggacccccaccccacctggcctcagtttctccacctgtggtcagacctataccagagacacgcagagggcattatgcgcaggagggtcccctcatgtgtgaggggcccaggtccaccatggggctggaggaaagcagccttcttgcctgcaacccaacgagggaaaaaagaagaattaggactctgcccccagggcctcttctggccctggcctacatctgagccaggctcttccaacccagtccaggtgctgggtccctgcagggtagacggccgtggcctcctcctccaactcccttctgctggctgtcactcctgtcctgctggaagtcccgcagccaccttctctcccctggggctgccggagcactagcctctggcctctaagaagagctgagggctgatccccttcaccttccgctccctccaccctgctccctgcctccactcctgcgtcctttccgaggtcctgacccgtctttagtttggaatcccaaagtgcccacctcatgaaaccatcccaaagctatctgtcccctgaggctatgacccacttccgagcagcctagacagccccaggctactggcttccttaaaggacagtcttctgtgcttgtttggtggggcaatgcgaagggacgtcaccctgggctctaggctctgacctcatttccctttcaccgcagagacaaatcctccacccccacctggcttcactctcctccagcggaagaagaattcagaactgactgtccttggtggttagctggctttgttttcaggccagagaggcgaatgattagttaatggtgttaagtgaaggaagggggttgggcagggaagccagctccatgcccagccgggcctgccagggcaaacggcatccttcagccattcccactccagcccagcagacaaaacattctcatctagtcagctctgggcctggggccagtccatacattcactctggagggcgagccagcgtcctcctcttgttctccctgcaggctccaggctccaggcccctggcccggtgggcctgggctctgcacagcctctggctggagactaatggcattggaggaggggtcgagaaggctctgaaagacaagcctggtgtggttttccagtcatctctgcgtcatcaaaacgccaccccggagcatgctctcaagtcacctggccatctccagggtttgctggctcaccctccacctcccccagcgagcaaaggggtggccccgctgggcaacagacctctacccaaatccaggcccctatttggcactccctggtggaggcgcgccagcagatgtggaccccttttctgaggccaggctgctggggtcagagtgaggcaagagcacctccccctccctagagccttagttcaagaggccagaagacccagggagaagccggcgttttgggagaaggggagctgcagagctccccacccaagcaggtctgatttgattcttccctcagctatggacccacaatcaatagggctggggaagacatggggtgggggaagatcagcgctgccgggatcctttcaatcatcaggaacagcaacaggtttgcagggtcaggctggggaccctcgcccattaactctttcttctccctgtttctttctcttaggtgaggggaaactgagttccagggtaggctccagagtgaagagggaagaaacatgattctcaaggcccaggtctggacaagtgtgaacaccttgggcctgcgaattcagccccctccttcctttctctggtcaaaggctagacttgcaggagcttgcgtttgaagggacagcccagaaggcatcgtctgcactccccatacaggtacttctgggtctgtgggactggcgcagggttcttctcccaaagctgccagcactgaggctgaggcagtgtcaggccggcggcagcggcagtggtgcaatcgttctgggaagcgatagaggccggcctgaattctctgtggcaagggaggggagcccaagtgggaggccccttggggacaccgaggaccaggtccgctactgctcctcccccaggaggtcccctaggggctacattggctggcaggggctgagcagcggtgagcctggctggcttcgacccggggcgactccgggcatccgggacagcttctcctcgctgccacctcggccagtcagaccccgagacacctgtcactaccccctcagccttcccaagccaggagcctgggagtccggctctggcctacctccggcagcgctcctaggcgcagctcccgggctggcggcgccggggcccgccccctagggctgcggcgcgcggggcgggggctgggggctgcgcggggcggggcgggcccgggcgctccgggccccctcccccgcccccctgacgtcagcccccggcagcctcgagctgctcacttggctctcgccctccggccgggaagcatggggcttcccaggctggtctgcgccttcttgctcgccgcctgctgctgctgtcctcgcgtcgcgggtgagttcgctcgcaggggggccgcgccccggctaggggtctgcggtggagcgtgccagggagcagagccagcggcgcggcggggtcggggcgttgcgtctgggaggacgagcctcctccctgggtccccgatccccgggcgccttgcgcgcgagcaactcttctttgcagccagtttgcagccgggattctagagtatcccgggagcagcactcggaaggcggggaggaggctgcttctgggaacgagaaggggtggagctcagcctttcggggtgctggggggtgggtggtccctgaggtgctcactctgggggcccgcaattgaagccgggcaggaggcgcagctggggcgcatcctcaaagcctgaattccgcgcccgcctgttgctggaaaaggcagcttccttcgctggagggggtgcgccgacccaccccttcccccttctgcctgggcatcacgccaggctggaggtgagcgagagcgggaggttcggcggctcccgcccgagctgggcgttggcaggggttgcggggcggtgtgggtcgcctcgcgcctccccgagtgatgggatcataggggacagagatgagggatggaggattcccatactggaagcgcccgctggcttattttggggaccacattcaggtgggaagtgcgcccgggcacctcggagcgtttctccggatccgcctggtagcagggcgctctcgggtccacgctgcccttgtatggcccgcagcggtgtcgcgtgtttctcttggctcccattccgccgtcccagccgctgtccggctggggaaggggagggctaggcaatacccagctcgctggcctcatgcccagtgccaaccatgtccctggggtattccaggctactgcctcccaggctgactttatttctggggaaagggctaaatcgggctccacagttgcagccggtccagctccaccctgccctgctcttctagctctgggaggagtcaggggtctgaggctctgggttggagaccccaccttccacctgccctccttgtccgagagccaaggtaacaacccaggcatcccagagcccaggcagatggtgtcgagtgacatcacctcctcacagggctggcagcacgctggcaccactgacgtcactcctgcccactgcctggcccttgccctgacccctgggggagactctgacctctccatccttaccagctacctagggtggggtccgcgggtgtgtgcggagtgttcatggaggtgctgctgagggagcatgagaccggaacttccgccagagttagcccgctggggagtgagggcagggattttggagggcagaggatagagcagtggtgtcttcccctggcggtggtgacacaaaagggcctgtgtgccccagcctggcacatgctttgcattcccacactctgagctcacccggagaggagggggcctggaaggaaaggggccttcctcttggccccgagcctaggttgcccctttctgcccctctaccagcctcagctggagctgttcggtgctcagtctctgctcaatctctgcttggctccaaggacctgggatctcctggacggggagagggctggcccaggtggggcgcgggtcggggtgggggtagagcgttcagaggacagggccctctgcagaccctctgagtggcaggaaaaacagctgcagcagcgctgcgaggggaggggcggacagcagccggagctgacacagctgcgggggccccgcctccctcccccaggtgtgcccggagaggctgagcagcctgcgcctgagctggtggaggtggaagtgggcagcacagcccttctgaagtgcggcctctcccagtcccaaggcaacctcagccatgtcgactggttttctgtgagtgcttgggctcctgcaagagcagcccggggagggagactctgcccctcttgtagcccaggacccttctcctcctctgtgtctcctcccattaggtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacagtgcgtgaccctgtgtctgccagcagggtagtgagggtggtgggacgtggccagatgtccccctcatttctattttcttgtagaagctccggaggagccaaacatccaggtcaaccccctgggcatccctgtgaacagtaaggagcctgaggaggtgagatgtgtggcaggtgggctgggcagctggtgcgcagccgctgagatggggcttgaagggggctcggaggcagggagactcacaccctgtgctctttaggtcgctacctgtgtagggaggaacgggtaccccattcctcaagtcatctggtacaagaatggccggcctctgaaggaggagaagaaccgtgagtagccctttctcccaaaagccacccttgggccaggtgtggtagctcacacctgtaatctcggtgctttgggaggccaaggcaggaggatctctggaggtgaggagttcaggaccaccttggacaacatagggtgaccctgtctctacagaaaataaaaaaattagccaggcatggtggtgtgtgcctgtggtcccagatacttgggaggctgaggcaggaggatcatatgagcccaggaaatgaaggctgcagtgagctaggattgcaccaccacactccggcctgggtgacagagcgagaccccatctcttaaaaagagctgtccctatggtactacacaggaactgccttatattttcattaagcttttaacttaaaaaaattatacaaatggtacttgaaaatattttcatgagaatttaaactgacaaaaaatctagaagtttcttcttgcctgagaccccccctcccagaaataatctctgctatcagggtgtgttctttcaagcctatttctatgtatttgctcatatatagaaatatttctagaatgatataggcttctgtgttttattatctaaatcagtcattcttaaccaggggtgattttgtaccccctcctcctaggagatacttggcaatgtctggagatatttttggttgtcacacatagagggggtgctactgccatctagtaggtagagagaccaaggatgttgctaacatcctatagggcacaggacagcccccacaataaagaatcaacgtggcctaaaacatcagtagtgctggctgggctcacgcctgtaatcccagcacttttggaggccaaggtgggcggatcacctgaggtcgggagttcaagaccagcctgtccaacacggagaaaccccatctctactgaaaatacaaaagtagccgggcgtggtggcgcatgtctgtaatcccagctactcaggaggctgaggcaggagaatcacttgaagccgggagggaggtggaggttgcggtgagccgagattgtaccactgcactccagcctgggcaacaagagtgaaactctgtctgaaaaaaaaaaaaaaaaaattatcagtagtgctgagaaaccctggtctaagtggtggtgtatggtatacattgttagacaatttcttttatacaatgtttctgggtcagtctatttagatcaactgatcgttttgcttactgccaagttttccatactacgcatagcaggtagtcgagttcaccattccccatttagtggacatctagacggctgctcgtttttatcattgcagcattctttgcacacatccttggatatgagcagacatgaaaatgtttttctagggttgacactgagcagtaaaagtgctgggttgaagggtttccagcttgcatttgtacctggccttctacaggggacagggggctatttagatggtcccctgccaaccccagtggacaaccctagggtggggctggaggtggggctgaggctgagtcttcctccccttcctccctgcccaggggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctgtgagtactaacctgactctctgcttactggggctggggctgctggaaactcctgctggctcccccttatccagcccagggttgactcatgggccaagagctaccccctttgcctatgcagacccgacagaaaaagtgtggctggaagtggagcccgtgggaatgctgaaggaaggggaccgcgtggaaatcaggtgtttggctgatggcaaccctccaccacacttcagcatcagcaagcaggtgtgggggctttgtgctgggtgcaggtggggatgcaagcagagacccaggtggagtggcgggaggtgcactctgctcacctccccaatcctccctagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactgtgaggggctggggacccaggacagggggaccaggctggggcaatagggaactggccccaccctgccctgggtgacatgattgctgtacccccagatgtgtctgacgtccgagtgagtcccgcagcccctgagagacaggaaggcagcagcctcaccctgacctgtgaggcagagagtagccaggacctcgagttccagtggctgagagaagaggtacccaggcgggtctggggcaaggggctgatggggtgggtggggctggcggtgacaagaatgtgctctttcccagaccctctgacccatctctcatcccagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggtgaggccctccctctgggtagagaccaggtcaccccaagtggggtggtttttaagctctttgacaaaaagccacctgctgccctggggagctctggtgcggagggggaggcaggctagctgggcttgccacctcacctctccctacctccccaaggccccccttggatggcattcaaggagaggaaggtgtgggtgaaagagaatatggtgttgaatctgtcttgtgaagcgtcagggcacccccggcccaccatctcctggaacgtcaacggcacggtgagcgggtgtctcatctgccacactctcctaatccctgcctgcgctttgtggccaggaccagagatcctccccatcccctgagctgcctccttcctttcctcgcaggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctgggtgagggctgcatccctgcaggggatggcaggggcaggcccaggcacgggcagtaacttgcgcttctcctaacacccctccctctgcctcctagtcaatttaaccaccctcacaccagactccaacacaaccactggcctcagcacttccactgccagtcctcataccagagccaacagcacctccacaggtaagccaggcctggcaagagaacagggctgtgccagggcatcctttctgccctgtccctccccagagagccctgtccagaaaggtgagtagcagccccatcttgtcggccctggactggctgggccaacgatggtgacgaagtggcctggggcagggagtgacgaggagtgtctttgtggcgcagagagaaagctgccggagccggagagccggggcgtggtcatcgtggctgtgattgtgtgcatcctggtcctggcggtgctgggcgctgtcctctatttcctctataagaagggcaagctgccgtgcaggcgctcagggaagcaggagatgtaagcccaacaccggcccctccgcccccgcccgttttgccacccagcctgctgcctgcccagcgcccgcctgccccttgacagcaggagtggggccctgggtcagggtgctgcctgctccactacccactgcccagcttccatcagccacatggtctcatgacctgcttccctgccctgggctggccctggagcaaggagccctcatccattccacatccttcacagcgggcccttctcttcctgtggccctgagataccaacctctcacctccctccctcctcccagcacgctacccccgtctcgtaagagcgaacttgtagttgaagttaagtcagataagctcccagaagagatgggcctcctgcagggcagcagcggtgacaagagggctccgggagaccaggtaggaggcggttcctgtggccagagcctgccacatcctcagggaccgggggcagtagggactaacggttctctctatccttcatccctctttgtcagagatagggagcaggccccatcttgccttcttaacactactcacagatggcagggccaagcaagtggctgagggagcttggaggaagactgagaaagaccctgtcccatcaccttttcctgaccaatgcccgtggctctgggtgtggccatttcaccagtgcagtcagacgggggccgccctccttccctggcctgccatgcccatcctagcttcctccccaacgctgcccaccaaattcatcggcttctgcttccatctctccttcctctcggtgctcacaccttcaactcagcaccgtctccgtgttcccctcttctcctctacccacttttagggagagaaatacatcgatctgaggcattagccccgaatcacttcagctcccttccctgcctggaccattcccagctccctgctcactcttctctcagccaaagcctccaaagggactagagagaagcctcctgctcccctcgcctgcacaccccctttcagagggccactgggttaggacctgaggacctcacttggccctgcaaggcccgcttttcagggaccagtccaccaccatctcctccacgttgagtgaagctcatcccaagcaaggagccccagtctcccgagcgggtaggagagtttcttgcagaacgtgttttttctttacacacattatggctgtaaatacctggctcctgccagcagctgagctgggtagcctctctgagctggtttcctgccccaaaggctggcttccaccatccaggtgcaccactgaagtgaggacacaccggagccaggcgcctgctcatgttgaagtgcgctgttcacacccgctccggagagcaccccagcagcatccagaagcagctgcagtgttgctgccaccaccctcctgtctgcctcttcaaagtctcctgtgacattttttctttggtcagaagccaggaactggtgtcattccttaaaagatacgtgccggggccaggtgtggtggctcacgcctgtaatcccagcactttgggaggccgaggcgggcggatcacaaagtcaggacgagaccatcctggctaacacggtgaaaccctgtctctactaaaaatacaaaaaaaaattagctaggcgtagtggttggcacctatagtcccagctactcggaaggctgaagcaggagaatggtatgaatccaggaggtggagcttgcagtgagccgagaccgtgccactgcactccagcctgggcaacacagcgagactccgtctcgaggaaaaaaaaagaaaagatacgtgcctgcggtgaggaagctgggcgctgttttcgagttcaggtgaattagcctcaatcccccgtgttcacttggctcccatagccctcttgatggatcacgtaaaactgaaaggcagcggggagcagacaaagatgaggtctacactgtccttcatggggattaaagctatggttatattagcaccaaacttctacaaaccaagctcagggccccaaccctagaagggcccaaatgagagaatggtacttagggatggaaaacgggcctggctagagcttcgggtgtgtgtgtctgtctgtgtgtatgcatacatatgtgtgtatatatggttttgtcaggtgtgtaaatttgcaaattgtttcctttatatatgtatgtatatatatatatgaaaatatatatatatatgaaaaataaagcttaattgtcccagaaatcatacattgctttttattcatcatgggtaccacaggaacctgggggcctgtgaaactacaaccaaaaggcacacaaaaccgtttccagttggcagcagagatcaggggttacctctgcttctgagcaaatggctcaagttctaccagagcagacagtcaccctacttttcagcagcaaaactgccctgatgacgcagcacgaagggcctggcaggctgttagcaggagctatgtccccttcctatgctttcctgccacttggaactgccctccagacagcagctcaggctcaccgccatgaggaagggggccttggcatttggtcctacggcaactcgagtattgcctcccaaccccctgaatgagcaagaactcaagtctgctacaacaaaagaaactttatttaaaaatatttttttacagacatgggtgcttgaaaaagctctttagttaactaactgtgtatggaaatgaaaaaaaattaataaataacattagtaaaacagacttctaaacattaatacactggcaaaataaaatggacttaaaatgaaaatgaatcttaaagctctgctattctctgtaagaatatttacctt

SEQ ID NO: 8 - DNA, 36199 bp, Homo sapiens
  source 1..36199: mol_type = other DNA
cacatgtgcagatacaccggctgacagtgacagcacacttatgtgtgtgaaatgctttatgtgctcaatttcttttaaatctcacaggaaacacacgctatgaggaactacccacatgcccacatcaggcaggaggaccccggcgggtgcaagggtggaagtcgtctcttcatggctgcacccttggccagtgccagtccagcagcctctttcaaagaagcttttataggcagatagtttgtcagcccagctcgaaatgtgaacaaggtagcagccgagcccccacccatccggctgcactcccctgcatgtccagccccgagccctgtgtcaagcacacctccttgggaggagaccgccccagcctgcaaggaagtgctcattatggagagtggagagtgacaggccactcccggccacgtctccaagcaagccggtgccccctcctcctgctgccaccgcccactgagctgcctgggctgcagacgccctggttggagcgtctggggccagatgttatagggcaagctaaaaataaccgggcaggccagccaccatcgaggaaggggcatgtgctggacgcggacacatgatccgagggaccctgctgggtggaactaagaaagtccagcagactgtgcacgctcctgtccccactcacaggcccacgcagcgaggggggcccctcttctgtgtgcgtctggaaggtcgctgcccagggaggaaatgcctttaaccggcgtggggccgggcagctgcaggaggcagatcatccgggctctgtgcctcttgctacttctcctccacgccggctctgccaagaatatctggaaacgggcattgcctgcgaggctggccgagaaatcccgtgtaagtgcctgggatggggaggctggggcagcctgtgtgggtgggagtcagccttgaggagaggcgatccctagacactgccttctcctgtaagtgctcccccaaaacgtggcaaggcaggcgtcttcctatccgtgtgtgtctgagttgtgtggtgggcagatggggctcacccccaaaaaaggacccttatctgtgaatcaagaggcaaaagacacatcacaaatgtgccaaagaatcgtgccttacttttgccttgttacaaaagtatgcttgctcctgaaagacagcatctcatttcggaaccccaggatttaaacggagcattccgtctccctgatcagtaccaaatgtttgtgaaatacacttatgcattcatgtggttgtttattcattcagcaaacagaatgaatacactctgttaaacagaatttagcagaaaccgctctggggaaggcactctgtggggtcctgtggatacaagataaaggaggtaccagcccccatcctcaagggtggcgtggccgaggtgcagaggcagttgggtgacaaacgatggcagtacaaagagaaggcatgaggcagtgagtggcaagaaacacatgccctggggccagatgtctgggtgtgaatcgggctctgctgctcattagctgcgtggcttctctcaggcaagctgcttgacgtctctgaaccccagcaatctcatctgcaaaagacagagggatgcatttcttcacacaactatgtctgggtcgccactgtttaccaggtactgtcctaggctgtggggatacaggcctcacatctctgcctggggaactcaatggggggctgggggcagcaaagaataaatgagagagatctcatttattagtatgtgcagattgcaagtgctgaggaagcaataaagcaggaagggaactaggagagagaatggggtggaggaggctatcatttttggttcctcccaccccccagagatcacagatccgcagggacggagtgagccaggacgatatcagcacacagagggctcaagaaggtaatgctggcacccacagcacctgtgtgaggattgaatgtctcagtatgtagaacgtctttgcaacatagcaagctggcagtgcatcttggcagctttcaatcaaataattgtgactttgtttggtgttgcctaacagggtagcatgtaggttccttggggtagggcagggttctgtttcactaatgtgtgcccctggtgcctgccattaagttgacgcctggtacattcttgtgggtgaattatgagcggctgcagtggtagcacgggatgcgtgtgcagctcttccccaggagagtcagacagggtttttagggttggggacagtggacctgtggctttaaaggataaataggagtttgccagctggagaagagggaagggagaatgattaatgaccccaggcatagtggggtgcctgaaactgtattgagacaaatctgggcctccagggctcatattctccatgttcggcacatgctcgtggcgggagacaggaggacttcgagaagtggaaagagtagtagagtgacatgatgaggcgcttggagaagggaaagctcatgtgagatggtgtagtcacctaaggcttcctggaggaggtggagaccttgactggatcttggggaaggattaggaattaggaggaggaaggaaattcctggaagaaggctcagcatgagccgaggctcacaggcagaagtggtgcatctaagagctgcttgggagaccagacctggccagaggggaggcattgaggaggggtgagagactcttctcagattttattgactcctccatgctcttctctggtgcaacaggatgatttaaccctcaactgtagttagtagcactttggttctacaaccatagagagattgccaatatgtcatctgattttatctccaccatgtcccaagatgtcataggagccagcactgtgacatccacataggagggtcagggttgatatgcgcgggggatttgggggagtctctaaaacacaggcctgggtgggctgcaggagctgattcacttgtagccaccactggtcaggatgcaagtgctttgaattggctcctcgctgaattgaaactttccctggttatcaacagatattggcctccttcctcagcgcctagacataggtgaggtttatgcagaacaactgtgccagccaggcacggtggctcatgcctgtaatcccagcactttgggaggccgaggcgggtggatcacctgaggtcaggagttcaagaccagcctgaccaacatggtgaaaccccatctctactaaaaatacaaaattagctgggtgtggcggcaaatgcctgtaatcccagctactcagaaggctgaggtggaagaattgcttgaacccgggaggtggaggttatagtgagccgagattgtgccattgcactccagcctgggcaacaagagtgaaactctgtctcaaaaaaaaaaaaaaaaaaaaaaaaaaaaaactgtgcctcagagaaagccctgggcgaagacaggctgttcgcctgtggctcagaggacagggagggctgggaagagcacctggcctggaacttatcacagtgaaatctctaatgtgctgtgcaatcttggaccactcgctgcccttctctggtcctcagtcctcacctgggaaatgaagatggagaacctatgactttcatggccctttcgttcgacagggacttacggatgataggaagcagcccccacatgcacaaagtcgagacttccccgtgaggcaccactccctcaccctacgctgagtgagaccttcagccttggccctgaccctcaagggctatgggctcgtcagagtgaggagatgctggctgtgtgctgaactggcttgcaccccctctggagagccagttttgttccacacagccatgatcacaaactaagttatataaacttacagttaaatcatttgtattaaaaagaaaggaaagaattttttttttttaagacagagtcccgtggtgtgatcttggttcactacaacctccacctccccggctcaagtgattcttgtgcctaggcctcctgagtagttgggattacaggcatgtaccaccacgcctggctaattttttgtatttttagtagagatggggttttgctggtctcaaactcctgagctcaggtgatccgcccgcctcggcctcccaaagtgctgggattataggtgtgagccaccgcacccagccaggaaatacttcttgttagaaatgcatcactttccaattgtattgctccgttttgctattctcttggctcctggggttttatgtcttttgcatccgtgtggtatgaccaggagagaagctactccacatctcttcccactcccattcagtggcctcacgtgggttgcctgaaatcaaccatgctgggagtatttacaccttggaaattggccagggctacatgaggagagctggtggttcagtcctgaccaagcgcagggctgcctgtagcctgctctagtctctcaaagcctcgtcccccggcagagtgagttttgtgtagcggactggtctcttagagtggggccggttaatgtcctgagtgctgtttatgcttcttggtctaagcatgcagagacagtgccagagatgagcacgcttgccttccattcccgtgggaggcccttaggagaagcttctaatgagaagtgccgggtctcccgccttctatctctaagaccttcaggagtttccttctcctatctgagcctcggtttcctcatctgcagcacgggacacagtagtgcctgctttgcagggctcagtaaaaagaccatagagcccacagttccctgactggcacagaactgcattccctacatgtgcactacggacagatttaaaaacccattccaggccgggcacagtggctcacagtggctcatgcctgtaatcccagcactttgggaggccaaggcaggcagatctcctgaggtcgggagttcaaaaccagcctgaccaacatggagaaaccccgtctgtactaaaaatacaaaaaaaattagccgggcatggtggcgcatgcctgtaatcccagctactcgggaggctgaggtaggagaattgcttgaaccctgggaggcggaggttgcggtgagccgagatcgtgccattggattccagcctgggcaagaagagcgcaactccatctcaaaaaacaaaaaacaaaaagcaaaaaaaaaaaaccaaaaaccccattccagaaccaaatatttatgagaatgaataaaacagaagcagaacgtttgtaaatagtccaataacctttccatggagctcagtcggttctgtggacttggtgtttttggtttttctttcctggcattagatgaaactcctgagaacaatgatttactgagcgcttacactgtgtcagggcaagagtttgccacgtattcgctcttgggagcctctcgacaaatccaccgggtggggcccatggcagccaccatgcgggcctcggtgctatgcagcccctggcctgggcaggctggcccctactgtcagtgtgactgtgggtctgttttcccctctaagcacccctccagggcagctggaaatggtggttcagtttgcaccctgcccagagatgcctgtcctgctgaggggggctgggaggggctagagcagccaggagctggtgccactttctctttcaaatgcatcctgccagacggggggagggaggggagcattgcattgtctatctggaggcaccccctgtgctaatagcagtaggggactgtaggagccagcagtggcccacaacttgcattcctcagttccccatctcctttcccccattatttctgtgagctcttgagagccttccaaacatccttttattttttgctgagatagcaagagtgggagaagcagatggcctggagctgtgctttcaccattccatgagttctgaccttcaccttcatctttgtaccgtaagcatctgtgtctgctggcctgaaggctttctctggctgcaggagcctgctcagcccacacacagtcaggccaaggtgccggggaggtaacacctgtaggagcaaatggagctggagcccgatggcccagctggcttctcaactttggtgggacacgagtgaggagtgttccacacggtttctccaaggtccactgggtttgagctctagtgcccaggcagccacctgctcattaacactccctgaagggcgccttccctttcctgcccagcttcccactccccagctggggcttcctgggatcccctcccaaatgtactccctgcactcgaatcctgtcggatccatgctgtgggttgaactgtgtccccccaaagatatatccaacccctaacttctgatccttgtgaatgtgaccttttttggaaacaggttcattacaggtgtaattagtcaaaataagatgaagtcatgctggaggtgggtggttccttaatatgaaccccgttccttataagaaaaggagaaggggccaggcatggtggcccacgcctgtaattccagcactttgggaggccgaggtgggcggatcacccgaggtcaggagttcgagaccagcctggccaacatgctgaaaccctgtctctactaaaaatacaaacattagttgggcatggtggtgcatgcctgtaatcacagctactcaggaggctgaggcagtagaatcacttgaacctgggaggcgaggtggacgttgcagtgagccaagatcatgccattgcactccagcctgggcgacagagtgagacttcatctcaaaaaaaaaaaggaaaaggagaagagacatagaatcagagacacatggagagaaggccatgcaaagacagagggttgcagaggtcagagtgatgtatctacaagagacacatgagggaccagagaacatgagggactgccagccaccactggaagctgggagagacattgaaaggttttctccttagagcctccacaaaggaaccaaccctgccagacacctggatctcagccttctggcttccagaactgtgaaagaatccacttgtgttgttttaagcccccagcgtgtggcattttgttgcagcaccctcggacacgaatacagtctgctctgggcatcaagggagtcagcttctgttggttgcaaccggcaacgagacatttcacagataaggaaactgaggccattgcagagaatgacttatctgtgctgttttggctggggacaagtggggctatcactatatccccagagtctgggagctttgtctgctccttgaaccaggtgagctcacaccctgcgggacttcatcatcaagactatgaagatgcctacgtcttgggggtcagcgggtgctcagtaggctccggaaacccccagcagcacccaggagggggtcttcaaggctcatggttgagaaccccagaaaagccccggagctgcctgggtgctggagagaggggatatgtcgtactgatgtgaagcaaacttactgtaggcttggccacaccctggtgtctagcccagaacctggcatggaggggggactggaaggaatgaaaagtccatgaatagctgacttttgagggggaccttaggcaaatcactttctcctcgctgggactgagattcctcatctgaggtcccagccctgaaattccaggtggaaatcaacgagtaaagaagcatcccaggtccctgtgtggttatcactcgggagagggagacaggaaagcaggacacctctgtgccagcactcaccgcagccacagggcagaggcagatggatgcccatggggtgagcacatcgcacctgtccactcaggggactagaactcagccatagaaaggtacggagcctggacacacgccattgcacagatgaacctcgaagacatgatgctgagtgcacaaaggccacgggtgtgtgacactgtttggatgcaacgtccagaagaggcaaatccagcgggacgggaaggagattcttggttgccaggggctgggggagggaggaatagggggcagctgtctgatgagaacagaggttctgcctggggtgatggaaaagccgcagaaatagatcatggaggtggttgcacacatcgtaagtctaattaatgccgccaactcgcacacttaagatggctgaaatggaaaattgcatgttatatatattttaccacaataatttttctaaaaagcagcgagaacctgaggcacctctcgggaattctgcgatggcacatagagtccttgtgagtcctctgtgtccccagcaccgagaggcagcctctcagcaagtgtccgctcacggtgcccactccggctcgtgggtcatcgtagcttcacgcccatctcctccgaggcctctccctgaatgccccattatgatttcccttaaatgtttacataagagcccagctcttccttcttagcatttagctctgttgttgttattggcatgatttgtttaggtttctctctaactgcttttgcagatgaccacaaaaagcaattcgttcgccttatctgtgtgctgttgtgtgtggtgtctctgacttgtcctgaaacagccacgtgaagtcagggccaacgtcttctggttcaactcggaactccccatttccagcccagcgcccgaaacccagtgggcctgggaattatgaatttactgttgaatgaatgaatcaattatgttaaaaccttccggaacagaatagaagggatggaaagttctagaggactctcgggtgcactgactgacaatgacattgttgggaagacttggtcgactttgccgtctcgtgaatttctttctttcttttttttttttttttggcctccctctgaggaagtcccattaaccttgtgccaaattctgtccccagagcccagtgttaagggccccgggcaccaggaaatggcattcatctacgcacgctgaactctaagcccactgttccggggagcgacatatttttagatgtttgacgtccaaattccagaggaagagcaagtaatgcttagaggaaaactggctttcttcctccaccacgcaaaccagtgctggggtgcatttttattggtctggccgctaagctgctggaactttccaaagaagggtaaaagctcctgcatgtggaaggaagggggtggtgagcactgagggaggcatgtgggtagctttggtggcagctctggagattggattctttttccctaaatggaagtggtttataaattcttctactgattattaaaagaatgatgtttactgtaaaaaaaaaaaaaagtcaggcaacacagaaagttataaattagtagaaatgatctgaattcttcctccaagatgtaaactctggtgacattctggggtgtctgtctagattgtttcatgaagcagttacacacagcacacgtgttttctcttagtggtattttattcgtactgattcagatttcttttattgaaatgaaattcacatgacagacaattcaccctttcaaaggtacaattcagtggtgtttagtatactcagagtcatgcaaccgtcctaactgcctagttttggaacgttcccatcaaccctacaggaaaccctgcagccattagccgtcagtcctccttccctcttcccgcagcctctggcaactaccaatccactttctgtctttatggatctgcctgttgtagacgtttcatggaaatggaatcatccagtatgtgctatttggtgtctggcttattcacactgttttatatgttgcattttcacttaataatctgtgggaagtatgttaacatggtggatcgacatcttcctttctaaggcctgcctggtgttctctgtgccttcacttatgtatccagcctcttttggttgcacatccagcttgtttgctttaattgaatctggacttggacttgctgaattggggttcccactgctgcagcccaaaatgggcatttccccacctgcttcctagggatatcagtggcagaatggctttgcaaatctatttttgttcatctatatgaacaaaaggtcatatagtttcactcttgtcacccaggctggattgcagtggtgccgtctggctcactgcaacttctgcctgcgtggttcaaatgattctcctgcctcagcctcctgaatagctgggattataggcacccgccaccacgcctggctagtttttgtatttttagtagagacgggtttcaccatattggccaggctggtctcgaactcctgacctcaggtgatccacctgccttggcctcccaaagtgctgggattataggtgtgagccaccgcgcccggccaggaatcatttttatccaggtaaggcatacagaaccggagggttcttgtctaagttgtaccatgaatgtgtgctcagtggctactttgaaatagacttactgtacaatccctgagcaggaatgaaaataaaagctggaaatcacgggttgctctggggttttcaggaaggtgtcgtccgtctggggtgagtgtcctcctttctcagggatttcatgtggcattattttgttttcatctgagaggcaacataagctgggagggaagggctgcaaagagggggccaggacaaatgtgtacctgggggcccccattttgactccaccctgagctggggctgggccacaggagggcagagggaacactttgggagggggagagagcccctggactgaaagaatgggaagaaattgacaggcaccattgggaagcaactcccagttccaggctcctgggtcaggaacgcccatggagtaggacagacccaggttcgagtcctgcctctgactcttctcagctgcttgctcttgggcaaatgcttttagtttttttgtgcctcagtttcctcatcagtaaagtgggtttagaatagcagcagccccagagggtgatactgaagattcagtatgtgaggacccagcacagcattggcacacagtaagagcttactaaatgctgcctcccttgccataagcatgaatcacacagcccaggctctaagcctggtgtctgtgtatggtgactgtgtgcctccgtttcctcacctatgcaatggggacagcagtggtacctactggggagggctggaggcgacttcaggtggtcctccgagggcagtgctcttgcacatgctgggtggctgtttccgcatggccccttctcttaggaactctgcgagttgtttgccctggtcttatccagaaatgatgaggttatatccaatcccacttttcactgttgctggtgggtttctcgtacgtgtatctgcagcctcagctggactggagctccttaggtttcttctgcatctgctgagcatctaacctaacgctatggagatggtgggcgttcactaaatcatctgggagtgaattgagggaggatacagcggaaatgaagaaagactggaagatatgaagaatgaagttactatgggacgagaggcttctgagtccattgattaaaaataaaaggagccaggcgtggtggctgacgcttgtaatcccagcactttgggaggctgaagcaggcagatcacctgagctcaggagttcaagaccatcctgggcaacaaggcaaaaccccatctctactaaaaatacaaaatattagccaggcgtggtggcgggcacctgtagtcccagctacttgggaggctgaagcatgagaactggttgagtctgggaggcggaggttgcagtgagctgagatcgtgcccttgcactccagcctgggtgacagagtgagaccttttctcaaaataataataataataataataataataataataataataataaaaggaataaaaatcaggtgaactatggattagcattgcccgtgctgagccgagcacacgcaggtgtggtctcttttgatcttcaccacagacatcggcagcaggtattacttttgtgtccattttgcagatgacgctcagatacgttagtgattttcctgtatgatcgcacagccatggggttcaaacccagacgcgtcagactcctacacctgagcccttaatccctgtgcaaagcagcctttgatgtataagagatcatgagcttcggtcccttccatcttgggatatggctcctaagttcttgccccaaaggatgtggaggttcagctgtgaaaaagagttaggaagcgttccgagtagaaggaacagtgtgcggaagcctccaaggccaggaggagcagcgaatcctcagaaactgaggatggccgagtccagctttacattttgaaagcatccttcctcatctctggagtgtgggatatcaagacccactcccctaattcacatccgcattaagtgagcataggagtgtgatgatgcttgcccatagcacagccttacacaaacaaaagggctgcccccacacctgtgcaggacccctgctctgcagaacccactgcacagggcccatgtgcagatcagcgagttaatgcatgttaagcacttagaagggtgcccagcaaactgccagggtgtgttgaccaccatcatcctcatcttcatcatcatcatcatcatcacagcttttgttccacccgaagaatatttactgagtacttgctatgtgccaggcactgttctacgagccagggacacaggtaagaacaaaaccaagttcctgtccctaggagggagaaaaacagcaaacaaatgagttcataatacacagtccacccaagggtgatgaggtgtgtgaagctccacaaagcagggagagggggaagggaaggctgtatgagcagatcccaggtaggtgagagccgggcagatatggtgggtgggcggggaaagagccagtgcaaaggcccagaggtgggactgggtggctggagagaatgagtaaggaggggagtgctggaagagaggtcagagacgtggagggggtcaggggagtcaattccagtccgacctctaggccaagaaccttggctgttaactcctttaacccatggggccagggataaattggagggtaagctgttcatgtctctgagtaaaagctatttcccagaactccagaaaacgccctgtgtttagaaccctctttgagtgtcaccttgcgtgacatgaaatatttttcaaaacatgtaatcctttaatctgggcctctcagtgaaatcagtgctttatcactcacgaccatctgttgtttcagtcacgcagtaaataactcgggagatgtggcttctaaggcgcttatctctttgttctctccaaattctgagaaatgacatttgaattttcctctgagacggcggcacagctcaaattgatctctgcgtaccctcccatctgggactgccattaaggaagtcccaggactgggaggcatttgtggtgggtgaaaagtgttttgcttctgatcaccaggttccattaaatttcataaatgaggcttcatcgaaaaataagctgggcggaaaaataagccgccccctcccgacgacgtacggtgtattcttaaccaccctccgaaggaggctggggcgcctgtcatgttagattaggattaaaaattgcggaccccaacatgggtccaatgtagttaattctgcagcaagtgaactgagagctaatggcgtgggattatgcacctgctgcggctggaaagttattggtggtggttacaggggtgagggagattgggcggagaaaggggtgataaaaggtgataaaaggtccacgctctggtctcccctctgtctttggggaaaaccattcacctccttaaggcctcgtctccatatccaccaaatgccagcttagtggtcattttcacttttcgtgaatttaacagccaatgtttacaagagtaaaaagaagggttctctgggtgaagcagggatgggggccaggggcccccctcccaggctttcctgccgcaccaccccccgctcctgcttggccattcctgctctctgcccctcatgccctcagtctcaggccctgaggctccctgggggcagctgaaaacctctggccttggtgattccaccaattcccaggggcacccggcccggacctgttcccagggacctggtgccccctgcagggcactggtgacaccgtgtgcaatttgcagctacccagataggcacgcaggaaggactgcgctcggtcccagccaggcagggacagacacagcctctgctctcagtgtggggtggcgagggcctaaaaatgaagaatcatactagaaacatcccgacgagtctggggaaagctgccctcagaaggctgtttaagctgaggactaaagggtgatttggccaattgaagctgatgggaggccttccaggcagaggaccagcacgtgccaatgatgatcggtggcactgtgctagatgccagcctgagtgtagatgcccgcctgggtgtacactccatcctgagtgtagacaccagcctgggtgtagacgccatccttggtgtagatgccatcctgagtgtagatgccagcctgggtgtagacgccatcctgagtgtagatgccagcctgggtgtagacgccatgctgagtgtagacgccagcctgggtgtagatgccatcctcagtgtagatgccagcctgggtgtagatgccagcctgggtgtagacgccagcctgggcatagatgccatcctgggtgtagacaccagcctgggcatagatgccatcctgggtgtagacaccatcctgggtgcagactcaggataccccaggagggaagttctgtagggagaaactgatgcttgcccaaggtcacacagcaagtgagtggcagagaggatttaagccctagagccgggatttgaaccaaggccgttagctgtggagcccgctctggttgcccgtctgctctgccggatgagacaagctcagctggaggaaacggtttgaagagatgaatgtgcttggagggcgaggggaggagggagggcagtttgctggcatgtaaggacaaggagggtcggggcgatgagatgaggccaatgtatcctatatctaagttattaaatcgaaaaagtaacatgtacctggttggcgcagggctcggaggtcgagtggggagctgggactttgtcccgagggtgctgggaagcctctggggccttggagcacagaggtgaccaaatccacaggtgcctttcataaagcctctttgggctgcagtttggagaatggatggatttggaggcatcgaggccagggacgaggccactgtggaggcactgatggggacttgggcctggggtggggttcatttatagaatgtgcctggcagagggacagatatcaagtttccaggggccgccgtaacaaatgaccacaaactggggtgggaggggtggttaagccaacagaaatgaattgtctcatggttctggggccagaagttcagcatcagcttcactggactgagatcaaggggtcagcagagccgcgcccccccaactccagaggttccttccttgccaatccctgttccggtggctgctggcaatcatggtcactccttggtctgtagacgccatctctgcctccctcttcacatgactttcttgtgtgcatgtgtgtgtgcgtgcgtgtgtacatgtgtgtgtggggggggcgtgcttgtgtgtgtgtgtgtgtgtgatctccctctgctcccctcttctcagacacttgtgactggatttagggcccactggaaaatgcagaatcatctccccatgtcaagatccttaacctaattgcgtctgcaaagaccctttttcctttgcatttaggattccagggagtagggcctggtgtctttggggacctttattcagccaactgtatgtgcaggaggagacacaggaggctcgaggccctcccttgaaggggtcgctcactggttaaatgcgatggcggatttctattaagacaccgagaaagagtgacaagggagggtgggtgtgggacatagaagcgggggtggtgtggccctaaccctggcctgggagggctggtgtcaccgaagcacatcccagccttgagcgctgatttgagtctcggggaatggtccctctggaagacctgaaggaacgggcattcggcagaggatcagcacgggcaaaggcctggaggtgacggcgcccacactgtcatcaaagcagcttcagtagctttgtgaggttctgtgagtcaagtctagattaagccaggacttggatgatgtgccctcgctctgcatggagactgcctttaaattgaagaagaatgtgtacatggtaaaaagacattgaaccactgcagaagggtgtacaatagaaagaaatctctctcagctttacaccctattcttctctccaaaggcacctttcctaatggtttcttgtgtgatcttgtgtatccttccaaaaacgttctcagcctatgcaaatagatatatatatgttacatctgtgtgcatttttttaaaaacacagatggggcagagcctacacgctgttccgaaccgtggttttcttcatgagccgccatatcctggcgatctttcatatgtggcacattgagtcttatttcattttttgccaacaccttaaaggcaggttttcatggctgtctactattccaatttgcagatatgccctatgtactgggccagcacaagggggccagattttgaatatgttgggggaggacttgtggggaggtaagtaggtgaagcattttagattaaaggaacagtttaggcagaggccaggaggcagcgcaagccagggtggtttttttccaattaatctatcctgtatctaactctgtgtgtgtgtgcaaatgagcacaaactgctcaccacgcaccccattttcatagaagcagaagagtgttactgggcctggatggagtctggtgtcaggggacccagggcagtttgcctggtggcttaaactttctcggtcttacctttaaaatattgatcttattggtcactcacccatattcctatatgttaagtacagtactatatcttttttttttttggagatggagtctcactctgtcacccagcctggagtgcaatggctcgatcttggctcactgcaagctccgactcccaggtccacaccattctcctacaggtgctcgccaccacgcccggctaattttttttcttttttttgtatttttagtagagacggggtttcaccgtgttagccaggatggtctcgatctcctgacctcatgatccacctaccttggcctcccaaagtgctgggattacaggtgtgagccactgcgcctggactttttttttttttttttttttaacagggtctcactctattgcccaggctggaggggagtagtgcgatcacagctcactgcagccttgacctcctgggatcaaaccatcctcccgcctcagcctcccaagtacctgggactacaggagagaatgagcatgcctgactgctttttttttttttttttttttagtggagaaggggtttcgctatgttggccagcctggtctcaaaacttctgagctcaagtgattcacccaccttgacttctggaaatgctgggattatacgcttgtgtgccacagcacctggcccctatatcttatttttgaactttttattaaaacataatacttcgggccaggcgcggtggctcacacctgtaatcccagcactttgggaggctgaggcaggcggatcacctgaggtagggaggtccagaccagcctggccaacatgtagaaaccctgtctctactaaaaatacaaaattagccgggtgtggtggcacatgcctctaatcccagctactcgggaggctgaggcaggagaatcgcttgaacccaggaggtggaggttgtggtgagctgagatcgcgccactgcattccagcctgggcaacaagagcgaaactccgtctcaaaaacaaaacaaaacaaacaaacaaaaaatacttcaaagcgtataaatcctacgtgttcagctccatgaattcccacaaaataaacacaccatgtaaccagcccccagaccaagaaacagaacagagccagtcccaagaccacgtgttcccagccagtcactgcccaccagattcctgacttctcatgccagacatcagcttggcctgtttttgagctgcaaatataagtggaatcccactgtggtgttctcttttgatctggcttttttcctttctttattttctgagatgtagcgatgttgttgcatggatttaatgattctgtgtataatttgtaaattctcaaagctctatataaacatggattctaagaaatccacacatatgtacatatacatatgtacatatagacatatgtgtgtacgtatatgtatacatgcagacatatatgtatatacacatatataccacatatattatgtattatgtatcatctctctctcacaccgggtagatacttaagtaattgtccttcagcaaacacaccaagatttgcttatccttttgctctgggctttcagttatttcgtgccgactgttataaaaagatacatggtatcttctgaccttctgagccgccctccgaggctggctctgcctgcttgctgtttctcacaagaggaaacgcaggctcagagaggcccggcgcactgcccacccccttgcaagctccgggtggcgccgggactcgaacgcgcgccccagagctgctgacaccgccctctccccgcaggccgaggaggcgggcgcgcccggcggcccccggcagccccgagcagaccgctgcccgccgcctccgcggacgctgccccccggcgcctgccaggccgcgcgctgtcaggcggactccgagtgcccgcggcaccggcgctgctgctacaacggatgcgcctacgcctgcctagaagctgtgccgcccccgccaggtaggtcctgggcccgagggagggggctgagggaggaggacaggtgaacagagctgtcccgggggaggggaagaaaggagctgggcttaaagctgggccacctgggcgggtctcgggcgcctccactgcgcacctgtgaactgggacgggcgctccttgagcaggcgtggcgctgctgtgcagctgctgtgtgtcccactgcccctggggacttgcgttgtaggagatgcccttccagagaaaatgccacttcctatgaaaatgggatgggggcagggaccctagatgcctccagggagccacagtccagcagacacccattcccgggaaaatgcccgctgataggatgcgggtgcagtgggatttctcatttaaaagatcaagattcgaggcttttctttaaaataccggaccatgttccctgggctgccaccagctggaacctcaggtgcccctggatgaggaggtgtcctctagcttcccactcaccccatcaacccaccaggcccgccacacttctcatgcctggccctggggtatttgaggtggagaccaggagcttagcaatccagatggggcgtgccccagagaggagcgggtgcgtggcttcagatccaccggccagctgtcactttcttgagtctcctctcctgtgaaatgggtggatcacctgaggtcaggagttcaagaccagtctgggcaacatgctgaaaccccgtctctactaaaaatacaaaaattagccaggcgtggtggcaggagcctcaaatcccagctacttgggagactgaggcaggagaatcacttgaacccaggaggtggagcttgcagtgagccaaggttgtgccattgcactccagcgtgggtgacaaagtgagactctgtctcaaaaaaaaaaagaaagaaaaagaaaagaaaagacaacgtgcagctcacggtggacctgccactccaaagtgctcagcactgcgcggggctgggagcagataatgagatgtgtgttgaatcacgatgccaacctctcggggagaaacaatgttcagggattcatcattggcaggacggagtttcagatcccttttgaagagaaggtttgggaagggaaggaggtgaccccgtcggggacagtaagtggggagtagggtagacaagtagctatttcctggggaaaatgtgcccttgggtggggaaaatgtgcccctaagcgggtaggatttgcggcaaggaagtcttctccaccctcggctctgctcagggtgataacaacagctgtttggactgtaccttccacgagaggctgtgggttcccatgggccttaattagggaaggcggctcgagaaggtggcccagcccctcctcccaaggctgagaggagagtcagagatggcaaggatgacctcactttgttttctttggggtgaggtcacagcgatgacaggggactcagcacacttaatcatgctcataattacagtcttataatgctcatcaagacagacagggactatttcagtactgcctctgggcaggaactggccgctgctccaggccaggcagcctcctactcagccttcataagaaaacctaaatgttgcccctcatggtggacaagcacataaaggcactcccctctgggtggacttatcaaaaatggtgccccttcctctggaatgtgacctgacatagggattctagccttgcctcctgcctatagcaggtgcttttgagcagtgcacagatgatacaactgtacatggcattcccaatagccttttcatacatcatctcgtttcacgctcacacagcttgatgagatgactgttatcagccctgtgtcacaactgaagctcagagaagctgaatcatttgcccaaggtcacacagcaggcagacctgcagtttccatccagttgtctgactgtagagaagtgttcattgccttaggccgtgagtgtgggaatgtggagcccctgccctggcacctgtacccacgtgtggggtccttcattgccaggaatatcctcccccagacatcccatggctcacttccttgcctcctttagacctatttccaaaggccacttttcaggaaagatttccctctctcaatgacagcccctccttggccatttttaaaccctccatccctgtttaatttttctctgtagcgtaatctggaagactgtgttttacttgtttgttcattgactgttctctccccacttgaaggtcagccccacagggcagggagtttgtctttcattcactgctgtaaccccagcatctaaaacggtctctgggcatataatactcaataaatattcactcattcattcattcactagccagcaggaagagcaatcatactactagtgagtaataacaacaatgctgtctaacaccagatttcacaatcttggcatattgacatttgggattggttgattctacttgctgggcaggggggcgctctcccgtgcactgtaggatggtcagcggcttccttggcccttccccaccagatgccagtagcaacctctaccccaagttgtgacaaccaaaaatgtctctaatctttcctaaatgctctctggggaacaaaatcgctctgcattgagaaacactggtctaactcaggccctgttccaggcaccttacttcttcattgccatctggatttgggtctgtccaaagcagacccagagacaaggacttgggcccaggttgtttatttgggaggtgatcccaggtagttccagtgaggtcatgaagagctgagaccccagggccaccaggcctttaagggtcttcaactagagtaatggggagcccggaccaagtttcactactccaaacatttgcttatttccctgctgctcagaagcctttgatcaaggcaaaagagaatacaaacagcacatattactacgtttcccacctgaaaatgtttcctaacacgtggtctgatgtggtggtggaaaggagggaagaaccgaaaggttgcgtatagaggctgaagaaccatcgcgtcattcctgttttggaggtcacagactgctggctcctttctctgctgttccaacttccatgtcattataatcagtataatcagcgagtgctcctttacagaaaaagctaaccagcatttttaccagtacccatcaatatactttaaaagatgctaattatttttctattctctttataaagataaatattagtaatttttatttttgtcttactttggagtcaaacgaatgcagaggtcacaagcaaatgtgatcccagaacagttttgatctccaacatgcttaaagttttctgcccaggcacggtggctcacacctgtaatcccagcactttgggaggccaaagcaggaggatcacgcgaggccaggagtttgggaccagcctgggcaacatagggagaccgcctatctctgtagaataatcatcatcatcatcattagtaaaaaaagttttaaaattacttgtcaacacttaaaaaccagaagatttctatttttaaaaatacagacttggccgggtgtggtggctcatgcctgtgataccagcactttgggagggcaaggcgggtagatcacctgaggtcaggagttctagaccaccctggccaacatggcaaaaacccgtctctactaaaaatacaaaaattagccaggcatggtgatgcgtgcctgtaatcccagctacttgggagactgaggcaggagactcgcttgaacccggaaggcagaggttgcaatgagccaagattgcgccattgcactccagcctgggcgacaagagtgaaactttgtctcaaaaaaaaaaaagaaaaaaatacagacttacagtccgggtgcagtggctcactcctgtaatcccagcactttgggaggccgaagggggtggatcacttgaggccaggagttcaagaccagcctgggcaacatggcaaaaccccatctctactaaaaatacaaaaattagccaggcgtggtggcacatgcctgtaatcccagctactcgggagactgagagaggagaactgcctgaacctgggaggcagaggttgcaatgagctgagatcgtgccactacccttcagcctgggtgacagaacaagactctgtctcaaaataaataaataaataaataaataaataaataaataaataaataagaagtaaataaataaatagaataagaacttacagtaagaaaaaaaataaaacagtctgggagcattggaaatgactggtcagggctaaccagcagctgattcatccaggcaggaggcactctctgctttgctgtggccctgcctggtctgttttcctgtcccctagaggcatttgagttttcgacccctgccttttgaaggccttcccataaactgtgtgggtgactctttctgttaaaaccaggtatattggttatacctgggtgatgggatcacctgtacaccaaacttcacccatacaccaaacttcagtgacatacaatttacctgtgcaacaaacctgcatgtgtacccccgaacctaaaaattaaaaaaataaagtattagttagagaacaggctatgctgctgcaacaaaaagacccataaatacagggacccaacctgtgaggcattttcttctctctcagctattagtgcagagacagacatttgtggtaggcaggtagctctgctcgcaaagccatctagggacccagcttccttccttcttgccactctgcagtctgactgatgtccttgtccacatggtcagaatggcccccagcctgcaggaaggaaaagagggagggagaggagcagagagccccgattggagggtgagacaggaagttgcatggtcactgctgctcacatctttgtagctgcaagggaggcagggaaggctagtccccagctttgtggcccagtgcccagcccacagtgaaaggttctatcactaaaagcaaagggtgaaggtgtgccaggaaccatagttatttctcccatggggagcctctgtgctgtcatgaagttggggtgcccccagcccccaagcctgggcgtttctcagctgcttgaggagggccctgatctgaccccgtattgtgttagtcttagactggctggtgcagccgaaacctcgatggcttggtggcaatggctggctcctggatggccctgaggaggtgttacaaggtacctgccgggtaaagcccagaccctacatccaagcctcgatccctccttctcagctgcttccagaaagctggcagcaccaggccggctgtcccccatgcacgggcccttcagccaaacactcagccctctttgatcctccccaccccagcgaagaagggctgatggctgggggcttggccagaatcaaagcccagtacagaataagggcttcgactctccccaagaatggagcctcctcctggatggagaattgaaggggagggggcttagggttggttttcaaattcctagaattctagggggttcaccgctctctaatgatgaagcctccctgggggtgggggaggtaccaggaggcagagggcaatttctggaaggaacagcatgcaggtcgggggcctggaggcctaggaggtgcgagcgggcacccttgcacacctgcggctgtgcttctgactccacgtccatctgtgtgttgtgtgactgtgtccgctagagtgtgcggccgtatatgcaactgtgtggctgtccttgtgcctgtggctgaatatttgtgtgtgtgtgtgtgtgtgtgtgtgtgtgtgcatgcaaaagtgtgtgactgtattgtgagtgtacatgtgtgggtaagcgtgtgtgtatgtgggtgtggatatgtgtgtgcaggatttgtgtgtggggatgcgtgcgtatatgtctgtgtgtatttgtgcgtctgacttgcttgtgtaaatgtctgtgtccccatgcgactgtagggctgagtgtgtgtttcagggtgtatccgtgaacatgtctatatggatgttgctgagcctgtgagagtatgtttgggatcaggtgagcttggactgcctgtgtttgtgtgcgtgtttgtgggtgtgcacgtgtgccacatgccgctgagtgagacccagggcctagcctggaacctggggtacagaggcgaggccgcctctctgggtgaggtgcgttccctgcacccgtcccgggagtctgccttctagaccctagcatgtgcacctgtcctgggagtcggccttctagaccccagcgtgtgtccctcctgcagcagaggcgtgcagcaccacggaggatggggccgaacccctgctctgtccctcgggctatgagtgccacatcctgagcccaggtgacgtggccgaaggtatccccaaccgtgggcagtgcgtcaagcagcgccggcaagcaggtgagtgtggcaccccagccctgatcctggctcctgccccagtccccttctccctgtaagcgcctctcacccgcatggacgacctgccccaggaagcagccccagcacctgggcctgactgagagctcctcacatcttccccctgcccggctccttctttcatggcccctaccctttccctgtcacactagcatgtgaccgaagctcttgagccagcagactggctgcaaaccccagctctgcacctcccagctgggcgacactggggttaatttcttcctatctctgagcctcagtttcctcatctgtaaaatggggctactacatgctgttgtggggcgtgactgaaatcataagtgcagagcagtcggagcagccctaaggaatagtaggtggtcagtaagcactggatgttttatatccacgtatgtgtagatggaccccgacttacgatggtcaacttaagattgttagactttacgctggtgcagaagcaacatgcattctgtagaaactgtacttcaagttcccatccaaccattctgtttttcactttcagtatggtaatcaaattacacgacacattcaatattttattataaaataggctttgtgttagatgactttgcccaactctaggctaatgtaagtgttctgagcacagttaaggtaggctcggctaaggtatggtgttcagtaggttaactgtgttcagtgcattttgacttaagatattctcagcttgtagggggtatatcagtacataactccattgtaagtcaagaagcatctgtatctgttttcaaactgcattgaattagaattgtctgagaatccttaacttccagaacttctgccaggactgttcagtttgacttaatttgtgttcgtctttggacacaaaggcaataaatagatgagtgaattgtgttgctctcattggcataattttcccaagaaaagaaagaacaatggcacagcgattcctggtggtatctaaaaccatctcgaaccccactcaaggcaatcaagttgaaagacttcaatgtgctttttggcatggagtttggatgaactgtgtggtgagccaggaatctgggacttcagggtggccaggttcaccttgtcaaagtcagtgtcagtcagactgagaccacacccaggggcccctgtccccgcttcggcatctcatagccctaatagctaaggacaggaccctcagacaagattggtgataattacagtgaccctatttgccgaaagtatactggggagtgcatgatctgccaaaacctctttttttttctgatttgagaatgtttttctctgaagtgtttcaagaaaggcatagacgtaaaatcagtcatgcatttaatgtcattatcattgcagataaagtgatgcaaatcccggaatcctgaactttgaagaaattatctacaaaaggctttgaaacaaagagatgcctttttggtttcctctacattccacttgctatttgcagggctttgtttggggttatagggtggcttctccagccagatagcctgggctgccacagctactcagccccttactgctgtgggcagcaaataacataaccttttcaggcctcatttgccttttctgtaaaatgggttcataatacttgaggtacttcacagggctctttgaggagtgagttaatctgtgtagagtgtccatcaattcacaagcacagtaaacaaccttcactgttttatcactgcttgtctctagacactcaatcctgccgtgggtgggtggggtgtatggtcctttggaggtaaaaccgcaagggcctgccagccactgtgaggctgctggagtgtggaagggatagccttttccaaaagagagctgtggggcgagttgtgagttcgttgggctatgccaccatttataataatgacggcataataggaatgccttgggcacttgtctttttcatcccctgatcttttcattcagccctatgatgtctaagaccaaggtcaaaccaagtcaaactagttcttaagggttcaaaccagttaaaaaaattcccatttcaagacagagctaagcaaagagggtcagtcattcagtggtgaccacaccagtgatgcctactgttaacatgcacctttggagtgctctctttaaattaagctttgctgcaagcctgcaaaaggagaagtagaaccaggaggcagtgctgcataggtttcagagtgcaggctcagaaccacagaaagaagtccaaagcctgtaactttcttcctccgtggccttgagcaggtcacccaacctctcttagcctcagttccttgatctgtaaaatagagatggcgatggtaaccatagtgactacttcatggggtagttatggggcttgagtgagagaaacacgtatgaagtgcttagaatagcacctggcccactgtaggtcctcagtgaatgtgtgccaccatcatccctgtcttcgtaaagggaggatatgagttcaggaatttacccaaaagatgcagagctggaagagctggaattccaagactgtccaccagcctcagagcctgtgtgtgtgtgtgcgtgtgtgtgtgtgtgtgtgtgtgtgtgtgtgagcttcccctcccaaatgaacgatttgaggcttccaaatggaactgtaagtggaattccccctagccttgttctcagggggctgacacttagcaataattactggtaatttactggtaattatatataggtttgaaaacattttgaattccagcaacttcccttggagcaaaaatctcaggggaagggtggcccccttgtcccagccttaccaaggaagaagctttttatttaagtttttaaaaactgcatatttgaaataatttcagacttataaaaatgttgcaaaagtagtaccttgaatcccccacccagattcaccagttgttaaaaaactaccgttacattttcctttatgctctctccccacttataaatctacgtgtgtatttttctggaccatttgagagtaagcgacaggccctgtgccgacacctaagatgccatcctcccgtaagagaggcttctagcgggtaccaatagtccttcaagtttgaccatccagcacctggaaccaggcatttgaggggttcacattttatgcaagaggaaaatgaacactgacatgtatagtaaatctggtttttcagagagtgggttcagggaaaatcggtgcatttttctgagtaggtttgaggtcacacagaagctgagctgcgggggctggcccctccttgtcctgcccaagacactgcttgagaagaatgaagtggggcttgtctagccttgagtgggaggcaatttgagaaaagtgacgagtaatcatttttggtaataaattataagtgattatatttggcctgaggagggggtccggggtccacatttacctctgtctccttgtggtgttatgggccagaattcacccccaggtaaagggggagcaggaaagttttggggagctcccccagcctacaaaaaccttagccatttgctttggaaaatagttattgctgcagttgctgatcagcaggcgctgacccaccggagggcctggagcggttccatggagcgttagcaccttgaccgataaatgtccgtgctggtcccaaacagcctgatgatttttagtatggccacaaaacaaaagaaaaagaaaatgtctggctaccgcgcatatgtgcttgattaaccttatagcagaaaaggcagcgctgatcaccaggatggagaagtgagctaaatttgagagagattgccacattcaagcatccacatgtcctggtcacattcctgtcagcaaatgctttcctgctaccatggggtgggtagcacggctagtcatgggacatgcatgaaattgccagcagttgaccactttttacacacaaagatagaattttcatacggctcatcctaatactgcatagatatgttttaaatgaaattgtttcttttaatcctcacaataactttaagaggtactaattttatgcctatttttaggagaggaaactgaggctcaaagaggacgagtgaggattcatactcaagttactttgtcatttgcagtaatgacatcagagtgtctcttgggcagactcaaaagagtgtcatctcttccccctgtgaacatattttgtaatgacatttactcaacagtgtgttgaaatttggcttccagttcatctgttaagacaatggttttggccgggcatggcggctcacgcctgtaatcccagcactttggcaggctgaggtgggtgaatcacctgaggtcaggagttcgaggccagcctgaccaacatggcgataccccatctctactcaaaatacaaaattgactgggtgtggtggcgcatgcctgtaatcccagctactcgggaggctgaggcaggagaatcacttgaacccaggaggcagaggttacagtgagctgagatcatgccactgcactccagcctgggcaacagagtgagacttcgtctaaaagaaaaaaaaaaaagacagtggttctgaaactttttgctctcaagaccgctgtaccctgaaagatgttaaggagatctcacagggctttcctttatgggttgatggctctggatattatcatattagaaactagagctgagacattacaaatgctcatgtagcctctgaacaattccattgtacactagtgagatggggagactgaaaaacacaagtaattcctcagtgttattatgacaacagttttggccttctaaactcctaaaagaagttttttcctctcacttgttttccagatgggcgaatcctacgacacaaactttacaaagaatatccaggtaaaagaagaaactgttctttctttccagagggcacaaaaaaaaggcagtgaaagtttcttatgtgaagaaaaaaataaaagcccagggctgagatatagggaacaaaatgggacctgggattaagaaacaaaatagaagacctgtggtctgatggtgggcagggagttgtgagtcacagaggagctcatggcaaacatgacctcatgctgaagagcagagcctggaactgctcatatttaagcctctgctactttcattcagagaattggaaaacacgcctgataccactcttccattcatccatctaatcatccattgatccatccatctctccatctattatttcatccatccattcatccctccatccatctacccacccatccatctacccaccatccatccatccatccaccgattcatccatccatcctctcattcatccattcatatatccatctattctttcattcatcttcctgttttttcatccattcattcctctatcaatctattcatccatctattcattcttccatccatccacccattcatccatccatccacatattcatctatcttttcacccacccatctatccttttatccacccctttattccttcatccctccatccatccatcctttcatttacttacctattcaccaatcatccattcatctatccatccatccattcatacatacatcctttcacttatccatctatccacccatctaccttttttttttttttgagatggagtctcactctgttgcctaggctggggtgcactggagtaatcttggctcactgcaaactctgcctcctgggttcaagcagttctcctgcgtcagcctcccaaatagctgggattacaggtgtccgccaccatacctggataatttttgtatttttagtagagacgaggtttcaccttgttggccaggctggtcttaaactcctgacctcaggtgatctgcccgcctcggcctcccaaagtgctagatttacaggcatgagccactgtgcctggccccatccacccatctgtctatccatttatccatccatccactcatccatccacccacccatctatgcatccacccaccatccatccacccacccattcatctatccattcactcattcatccattcatatattcatctaatctttcatccatccattttttcaagcatgcatccatccatccattcctccatcgatctattcatccatctgttcattcttccatctatccatctatatattcatctatcttttcatctacccatctattattttatccacccttctattcctttatccctccatccaaccatccttatatccactcatctatttgcccgtacatccattcacccaaccatccatccatctgcccattcatccattcatgcacatatccttgcattcattcatctatctcttctttcatccattcatacatccatccatccacccatccacccatctacccatccatccattcacctatgtatccattcatccatccacccatatatccatctatctattcagccatccagccagccactatccatccatctatccatccatccatatatccatccgtccatccacccacccatctatccatccatccatccatccatccattcatctacccatctatccatctatccacccacccatatatccatgcatccatccatccatcatttatccattcacccattcacccatccatccatccatcatttatccattcacccatccatccatccattctcccatccatctgatatgtctttatcaataatttacagcacatattttctaagtattactcatcagtctgtgctgggggttggagctacatttgtgaacatgacagacccagtcgctgctgtcatgcagctaatatcaagcagaaaattcagacatgaatatagcccaagtggttattttatgagaatagtgacaagccttcctgacatcacatgctggatactatgaggacaccttagtccaggttgtgagaaaaggcttcctaaggaggagacatgaaagctcagcctaaagaataagaaatagtgggcagcagaatggcatgtgtgaagctcttgaggtcggaagcaatttggccatccccaaagctggagtgtgtggtgagctgtggaggtggtgggaggtaagaccagaaacatagacagagctggtgacaccagctggggggcctggggagggagaggaggcactgcaggtggggactgagtgacctcagctgggggagggaggagagggccaggtcaccaggcttcatttggcagttcctggtgagccacggggggcattagagcaggaatagatacatctaccccaacagagctgtgttcttttcacagaaggtgactcaaagaatgtggcagaacctggaaggggacaacagaagcactttcagtaaagcaacggcaagcaggtgagtgggcagagagcaagagtcatggccagggtaaatgtgggcagccagtgtcctggcagctttcaccaccaggttgaggagcaggagggtgagagtagcgctttccctactggtagaatttgaaattcccttctgcagtaagtcctcacctaacgtcatcaataggttcttggaaactgactttaaatgcaacgacatactatatgcactgtatgccatagaagcttaactcatttgtttgtttttttaagagatggggtctcactctgtcacccaggctggagtgcactggcacaatcattgttcactgcagcctcagactcctaggctcaagccatcctcctgcctcagctttagtctcagtagctgggactacagacatacaccacctcacctggctaattttttttttgtattttttgtagagatgagttctcactatgttactcaggctggtcttgaactcctggcctcaagcagtcctcccactttggcctcccaaagtgctgggattgcaggcatgagtcactgtgcctggccttaactcttgtttatatccattagcctatggtaaaattgtttttgttatatagtaggtcattttgcttaaagttgcagtttccaataagctatccatgatgttaaatgaggacctactgcaatttcaaggcaggaacgtaggagaactttgtgtgtgatcagaagcagaagcaattctgtatccctctgaatcaaaaatccaagaccagactaggtcgcaactttccttttcattcctttgccttgatgtctccaggaagtcttgtctatctcttctcatttctgtcaaacaattctgttgggtgccaactatctgatagtcatggttccagcctcttggttgctcagcggtagttcatgccagggtggtatagtgaacaaatcccacagagccgctgccctcagggaggccacatcctggtaggggacggctacacaagccaacagatcaatcaatacaaagacaggtgtggtgaagaagtgaacaggcaatgtggggcagagtccctgggcaggagcagagcctggggcaggggctgcttcgtttggggtcagggaaggccctgtgggggttgaagtctgagccaagatccaaggtgagaaggtgctgcttggggagggtctggaggataggcgcccaggtggagggagttggaagagtgaaagtactgaggcaggagggacgctggctttggggagccaaagaaggcccctggggtagacgcaggaaggtggcagggaggctgactccagaggaggccgcaggtgaggcacaggcttgccctcacgtgccctcaaaggtgaggcgtaggtgcatgcaaagggctgtggagttgcagccctaaacctgaaaatctacccagcccctgtgaatgctggatagccacagagggcaaaaaggagcccctaaaagcaggtgacagcctcccaggacgtcgctgaaacgcaaatgtcagattgcaggttccagaatcctcccggaaaatctggagcccgtagggactgatgccagcacgtctgttacctgctgatgacaggagccgtccctggagtaggtcagatgcagctgaggactgagcttctaacagggaagaaggaggccagtggatgaggacgagaccttgggaggcagttgaggatggcagtttggaagttgaggcttttgggtgggcgcagcagctcccgcctgtaatctcaacattttgggagaccgaagcaggcatatggtataatcccaggagttcaagcccagcctggcctggccaacatggtgaaaccctatctctacaaaaaaaaatacaaaactacccaggcatggtggtgtgcacctgtggtcccagctacttgggaggctgtggtgggaggatcacctgagcctagggaggttaaggctgcagtgagccatgtttgcaccattgcactccagcctgggtgacagagtgagaccctgtttcaagaaaaaaaaaaagtacagcctttggagcaagctaggtctgggtttaaattcttgttccagcactgtggatttagtaaataatttcggctctccaagcctcagcctcctcatctataaaatggaactgatggtaggggaggagttaggaactaaataagatgatgccgtgaagacgtcacagtgagttcagaattgggtcttctcagaaaggggcatcttcctggtgtttctgatggtggcggtggcggcggggagacgaaagatttgtgactctgtggtctgccctgcagtggttcaccctgacccttctttgtctctggtctgccctgcagcggttcaccctgatccttctttgcttgctttccagctaggttgcaagaacattcctctactttctgctaagccttggaaacagttgggaaaagtagtttgaccctcacagttcacattcagctcagcagagcaagaccccagagatgcttagagacaggacacctggccatcaaacccagtttggcccagcctggttgggtgactttgtgggagccacttaacagctctgggtccctgttttaccatcctgggagcaaggccctgcagctccacgagacctttaccccgggaagaagccgccacccatgaaagcatttctgaagcccctttctaagacaaggctcagcatcttgatatttttgacagattcctcccaagtctggctctgggaggtatgtacccatctcaaatgttcccaagataaattcatccttcaggaaatggaaatgaacttgcttactaatgtgtgattcctagttgtagccaccggatgtgctgaggcctaaatgttagcaggtgggaggaggccacagaacaataaaaacaaccaaataagatgccgagtctctggtgttttgtgatactttgtcactgtttaattcaggggtgggaagcagcgtgggtattgctgggatgaggtgtcagctaggctctgctcctggaaccctgtgttagcatagagcccctgaagcagaccttgggagggggatttgttgaaagtgattcattagggcggttcctgggaaaaactggcatgagagtggggaagcggaacaggaggagacagtcagacgagggagaggtgacgagggcccatggagggcacctttgctgcaatccctctgggggtcttggaggaagcctgagtcccaccagggctggctccagggctgtgcatccagaaggaccctatgcttggtttaatgatctgctgtcaccatttaaaaactcttagtcattgttgaacaaggggcctgcttgttcattttgcaaatgatgtcggtagccctgagccacactgtggagtcggtcccactggggctggacagcggaacccgagtatgcgtaacgcttccagcacccatcagtgggtggcgaggctgcccctccccctaagagaggaggcagcccacctgctgccagggagggagaggcac

SEQ ID NO: 9 - DNA, 87071 bp, Homo sapiens
  source 1..87071: mol_type = other DNA
atttcccaacatggtaccccaaagcctctcatgacacaatctgcctccctcattttggattttccaaaagattttactcccctgtcttcccagagaatgtgtcactccctctcattgtgcactgttcatgtttttaaaagagcacatgcccatcatatgactgtgaagttttcctctttgtctctgacattcagctgaaagcccctgaagggacttgaccatatcttattcctctttgttttcctagggatgttgtagttgggcacatagtagagctcacaaaatgttgctgaacatagtgcaccattgacacaacataattccagatttctaactttgatttctgaaaggtaaacattgctacaatgtattaaccacacagaagactcaaacaaacccaacccccttccaaacaaagcaggtttgtttgttttttgttagggcacccacttctacaaacaaatttctggccccaggataacacaaagagccagagtaggatttcaagcagaactgtgttttcagttgatgtgtcagtcccctgagagtcatgtggaaaaaaaaaaaaagaaaaaattcaaggtccaggttatttccaccactcctgggaaaccaggcctggagagctctctagggaaagaggtatgtctgctctgggcttttgcaaccttattttataattcactttcttatctactgcttcacaaaaccaaagggaaataggtacaaactgtatcgacaaaagatcagaactgaattctcaatggcaaaggcaagtgtacattataaatagcaaaacagctggcttggaccatgttgccggccagtcacccagttgagggatttgaatgacatcataaccctcgagagggtattgctagccagctggtgttatttagaatacacaaaaatcagagaaagaaaacacactctggcacacagactccctctgtcatacacacacacacacacacacacacacacacacacacacacaggtttgagttatatggaaaattcaaacaacaggaaaattgtttgccccccaggtacccttctcccagagtggtggggtggggaggggacagtgacaggcagcctagtagaagaataaagaaaaatgttctatttcagttgggttttacagctcggcatagtctttgcctcatcgcaggagaaaaagtatgagacagtgccctaaagggaccaatccaatgctgcctgcccctccataggttctaggaaatgagatcacacctctcacttggcaactgggacaaggggtcacccgagtgctgtcttccaatctactttaccccagtcacttcagggttaaaattgtagagtttgctggagagggtcttatcgtcctttctttctttttttgttttaaataatgcatttgctctagaatctaaaattgctctcccatcccccatattcctttaatactggtaaggtgtattagcagacgtttgtgtcttcatgcccagcagaaagttaatcagaaaacagatccttattttctatggcagcataagtattttaatgtctgcgaaccctgtcactaacacacattcttttaagggaaaaaaatgcttctgtgctctagttttaaaatgcaaaggtatgatgttatttgtcaccatgcccaaaaaagtccttactcaataactttgccagaagagggagagagagagaaggcaaatgttcccccagctgtttcctgtctacagtgtctgtgttttgtagataaatgtgaggattttctctaaatccctcttctgtttgctaaatctcactgtcactgctaaattcagagcagatagagcctgcgcaatggaataaagtcctcaaaattgaaatgtgacattgctctcaacatctcccatctctctggatttctttttgcttcattattcctgctaaccaattcattttcagactttgtacttcagaagcaatgggaaaaatcagcagtcttccaacccaattatttaagtgctgcttttgtgatttcttgaaggtaaatatttcttactctttgaagtcattggggaattctatttaaattgtgtactgtttgcttctgcctagaactgttcttcactttaaaattttcattgtttcggaaccgagagttatttataaattgctgaatatgcaattctgtggaatctgaaaaatagctcggggagatggatgcatttgcacagatatctgtatgagtagaaactattgcaaggtacttatgctaaatcctccacttctgcagggcttccgtggtgtcattacagaagattcctttaaatcctctctatggctaagggctatagagcatggatatgaacttggggattttttttttcttttgcaggtgcagatgttttatttttaagaccatgttcttttgcatgtgtgtatgtgtctctgtgtgtatgtgtctctgtgtgtgtaactcatcaggactttatttacaaggcatgctatatatgaagggttgtattttagatgatattaaagcttttaaatatgatctttggagctaaggtcccggaactctgcacttatgcccagagagagtgttaaagttagagtaaagtgtaatttgttcttccaaaaaagaactccttgacaactcttgctgaccttgcatatttgtataatttaaacaaatacatactgtatatggaaagcagaaactttctaagagctgcttgcccaacttttctgtttagaagaggactttcatgggcaaagtttggacttggggttctgtgttataaaactctgattttatattcagtgtcgtgaagtccctttaggtaaatctggctgctgctgtcagtgcaccgacttctcgtttccgattgctggccgtagttctagtttccattctcagcaaaattatatccttcaagacttgtgtttttttttcaatttgcaagcgcttttaagctgctgtcactggctccaccgattcaattgcctgagggctcaattcataagacgtctctgccacttagtgcagcattcagttgctgctttcaaacacttcaccactacgactttccctagtcaagtaagtggctccaggagttaaagatacacccttgtcaggcacctgattctgctgttcctgagatgccaacacatgcaggccaccttgctttcaaagaaatgacgtcactgtgcatacatactatgtgatctagcagctggagtttttgtctccttacttaggggatcataaaagaggctgtggagcgttatctctgcattaattacaagttaagaaaattgtttccaaatgcacttttcatgctgtgtatgctgaacacttcagaagtggagctcttaataagttgttacttttttctgtacttgaagcaggaagtggtttgagggagctgcgtggtctttcacatgtaattcagtgggtaaaggtgtcctgcccagaggcagagctcaccagctgattgtactctgactctcaaggtatttccaagtgagtgagtcgggggaagggagtaagggagtggactggagcttgggtcactatctgggctgctacaataggcatacaatggaaataggtggcttgactggggggaaaagattgacttaaatcccagctgtgcaatttgttcattgtttcaatggacaaaaggcagtttacccaggctcataatagcatacctgcctgggtgtccaaatgtaactagatgctttcacaaaccccacccacaaagcagcacatgtttttaagacttcagttttctattcacatcggcctcataatacccaccctgacctgctgtaaaagacctggaacaaacaaaaatgattacacctacagtgagtattttcttatgactgttgccctcaaattttacagggcattttcattgtggcccagacatctggaatcaattaatattccatttatctaagattaaaaaaaaaagaacttttaaaatttgggtttgtgaatgatttttgagaaagtgttttctgatttttttttctttttttctcatgtctttctgattcttcccttttttttctatcatatctttcctttctctctattgatttcttttgtgtttggcaaaataaaaggccaaggaaataatgaacatatgggaccacttgtttcacactttaaactcctaagcaagttcggtattgttttcatttgtgggaacataaaattctctggttctctgtgggtgtactggactgttccctacactaaaggaaaatgcactagaggttctgtcttgtcctagaactgtaagtactgagatttatctgacaatataactcagcaaaaaaaaaaaaaaagaaaaagagaaatctatttaaaaaaaatcttaaattcttaagttccagagacttatatgtatcgctgcacttaaatgtgttatactagaactattaaaaatatagttttgacatctaatgaagtttgaaattcactaaggctataacttgagtaatttttttccctggatttcacaaaaactgaagaacttgaaggcatctacagttgcaggtgcttagccttcagcattgctatgaacgcaacaaagaagggaggggtggagaaataggattcggtagaataacatactttctagaactggcccattcccgcagtgtgggggtatgctctgcaagatcaatcacaggtttgttcttttgaaaaatgccacgaggtccaggtgggttggatttgttgaggtttccaatggaaatattgaacaggaaaaccccactaattttacaaccaaaaaaaaaaaaaaatgagaactggatgataacatgcttcttccagtcccagtgttttgggctatgtagggggtggaagatttccatgcggagatcttgttgagaaaaatgcagtaaatgttcagttgagcttttacctcctcttctattttatgtccattatttatgatcatttgcaaatagagggcgcttttcatcttcaactgctttacaaccattaactgaatatttatattttgttaacgagagacaggggttttggttttttttttttttcctcagttgctgaggtgaaggtctgactacaaaaaatggaaatcatcattagaatgatggggaaaattttgccactgatatagagccactaaattgagagtccatggagggcaaggagggtagtcagaaaataaatgactgtttctccaaaagtgatggggttgaatggacctgccactactctgaggttcaaaaaaaatgctcttcttataattatagcttccagcatgactccacaacccttttaatagcgcccctcacctaacaccctgtcagggactcttcccatgatgctgaaagtagatgcatctgagagaagtgaagccactcaccttgagaggcttcttggaaaaagccacatctttgtgccacaagtaacaatatggaaaaacttaatgatcatcttagcatggtttctactacttagagtgaggatgaagacttgagccacaggactgctgcttttggcaaggctgccgtttggcagtctatcattgactaagtcatgccaaccaggggtcccttggtatattttgctataaaatagatacagtgacacataagtccctccaagagatgaagccataattcctgtgactaaaaataaaataaaagatttttgccagtttgagcccagtgccctatacacagaagggattctcaacacgtgttgatttgatattggacttgaggccagggactttagaatacaggagtggtttctatttgagccaatttcattcttcgattctcatatttttttctgtttctgttattttcttctatttgtggttgtgtcaactgctgatatgccccaggtaaggaatcagtctctctctctctctctctctctctctctctctcatattgaggaaacaaaacaaaaacaaaaccctgtgatgatatatagtattgtcaaaagccattttttggccatagtaaattatttttagtcaaaaatagggaatgatgttttggttaaaaatattctgctcaataatgaatgcatataccatctatggatggccaatattgagagaatttaaattaacaaaacttatttggccccaaatctgtactgagcacaatttaacctttgataattcaaaagttctaccagggtctcagagtgatatgggatcccactgtaataatagcatctttcatttccgtagtaaacgtttctagatattttgtctcaattcattgaaataggaacccataaagaaagggttcagggaggactcctccaaagatccacagtagccaggggaataaacacaggttgttggatgccgagacacgctccatccacaactccctctgggttctcatgtactctattggcttctgtgctgggtagtcctgattaatgacagtcgtggaatcgtgggagtcaatgcacttctgtcccaccccactccccttgcaaggatcaaggaggaaacctgaccctccctctgtttcttgggcaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcagtaagtagccctccctctcaatgtgctgctcaagtctaaagtgtacagctctgtggatttacaaccgcagggagtgtgtgaataactgaatgagtgcccgtatctggcagccatcctaggcctctgagattcctcaccttaaagtaagcatagtgttttggcgggactttggcaggttttgcagatctaggatggaattttcctccttaacaattcgtctttaactacttcctgctaagccttctttcaactattatgatccacctcactattccattcctttcaatgaaaggacaggacaaaataaccctgaggtattgatgtttttgttttcaaaacctatgggcgcacatataaacatatggaacacatgcagggctgctttaagtgctatgtgatttggagtaaaatagtaaggactctccagtcatgtggatctgggaaaaaaaatattaaagggcatatcctagatgtctccattcaatatgaaatcaccatgaacagacagaaggttaactccccttaaaggcaggcctgtttgcaaatacacagactgtctctaaatgcaaactgaaatgccatactgatttgccctctatgccacttgaccttgatttttttgttgttgctctcttttggcatagttcttctccgggacccatcaatgatatcaccatctggaaaagggtctctgacaagtacagaatcacttatctcttaaatagctcttaggtgattagaaagtcaacagccagtaaaaagtagacagccaagtcaaggcgtataaataatacacacaagcaaaagtcaatgattcctgtgggaactgatcacttaaagaatctagctcccatgcatctatctcactgtatattataggacagagaaagatttaaagccagatgcttgttccttataattctttggagttttagagagttccaggaaactgagcatgagtcattcttaaaggttcatatcactccagtgtagacagcttacctcagttacccactatgctttgaatagctcctgtgatgactcacacacttgaagtctctctcatccttcccaaatgtcagcctctctctaggcaggtggggtatgtgtgtgtatgactggcagggtagagggtggcctgtgtataccagtctggagatcagtaagggagatgggaagaaagaggcttgtgagggtattggcgatcagtacttacagtggatttggcaacaaatagtttttaaatcactagtgacaaagaatcagaagtgccttctactatttgaataggcaaattcccactatttgcttccagccagtcctttaattgaacgaactcatttatttttctggcaataaagcaattctatccagttctctgttgcagagcagatgaatgagtgcattgggtgagtgattttccaatagcagacttgttttacctttaaaatatcctgtggaagataactgtgggacatcacatgatccaacagtgggtcataattattctataatttgcagctactgatcctcattttttaaagaaatgaaaaagtggttctcacttattatgcccaaggggactttttttttccttgataatccatttgtgcaatttcctttgatccagttcactaggtaattgccaagcctagaagtgtcctctgttacgtgccaggttgaattgtgatgttcaacacgacattgctccagaatttccatgcacagtgcatgaaaactggatctttctatgcctatgatgtttgcattaaatgatgaaaaattagaatggaaaaaatgattgttcctaagtaactgtatgatacttataagtgtttgcacagacacttaaggatattggacaactaaaatggaaaccaatttccaggttctcttattctgttactgcaacaatcaaatttcagcaaatttttaaaaattgtatctcaataacttgaaaaggaaatgttatatatagcaaatcttgattcttactatatgactttagctagaaagtggctttgtaaatcatatgttcttgtcagtattgtcattgaaaatatgtgtgtcatgagttttgccttatagaattccatgacttatgttaatccgacataccctcatgcacagaagtgtccaacataccttcatgcccaaaagtattcttgctaactttctagtcaactggacaatcttatttgcccaaaagaaattagtccatttagattattttcatgtaattcaaaacattgatggagcatctgttatgtgtaagcattgaatgagatatgaatgagatgcctagaaatgaatgagagattgaccttgttttcaaggggcttacagttgagtggtaaaagaaaaatgcaactaagtattattgtcataagagaaaaataaccatggagaataaattatatcctttatgtttttcttgaatattttggtctaccctgccaagaataataccctgtcaatgggcttcttgatgccaaaggacatatttatttacatttgaatccaagtgtctagctcatagcctggatgctatagtagctgcccatatttgattgttgaactgacaggattgaattgaattgaattgagtctagttgaatccaatcgaatagaatggaattacttgtttgggatcttggtcttaagaaccatcaagaattaaggttgtcaagtactgtggacaatacttctcacctcatggttatctagttatttaataaaactgcttttatttttttttgaactgaaaaaatatatactaacaatgacttagaggctgctgcattttggtcatccccaggctacattgatgtctgttttgtgtctgagccaattattcctacaggcatcaattgttgtcaacaaatttcattcagtttccttttcatccgctgagatttactgtgtgtggagaagtcagctctaagcacatagtctttgaaatactttttagagggaagcattcttccccacctctatacactgtcagttgactcctattattctctttacacttattttcaaagttttattgtggctgtgacttttattttaaactgcttcaaatccattttggaagtacaatttttttagtagtagaaggcagtttctgattttagtttgaaattcaaacatctaaaagaaaattgcctccatatctatcatttttgtcaatctgcaatatttaacaaggagagtttttttttaactctattagaactgttctgaataaaatattttgtaatgcttactgtgtgtagattgacccaatgatgtgaattgagctcatattccccttcaagcccaagttccactacagcaagtgaaagcaaccttctagattatctgtttgtgaacattatcccatcataaatggaacagttcatgtataattaatgcagcttccctagacattttatgaaatgaaagacccaatgaagagctaattactcattctttcctatgcatcatagggaagataggagagcctatcagccagccttgtgttcttgcagtcctgtgagaaaaatggtggcataatggagatgaaagcacattgggaaggagctagcgattttcttactagattgatggagcccttataaagggtggctcattacaacctcattttgcatgaaaaacaccaagagaaaaagagcttcaaatgaaataggagaagtgaagcttaccatagatataatttttgacagccatggtgattaaccattttaacatgctgctatgggcctgtggaatttctctccttggagattcttaaaaaattgacagacaaccaaccatcttggatggtttcaattaagagcaactgaaagaaagagagtttctggggtctttttaagttttaagagtctataaacagtcccttaaataaactgtatatgctttaccttggcggtctggggaaatggagaagtaaatcaattagaaaagccaagttggatattctttcagctcttcaggatttatagcttttgtaaaacatacttccaatgtatagttattaagaacaggtctgaataaacgaatggcattcttcatgggttaaaacagactgttttgtaagagctttaagtgaaactttacaatgaactttcattgttattgattgcaacaaccttcttagatctaggttattattattaagtcaagcttctttcctttgatatatactgcccagaaaactactcccagacctgtcgttcattagttgtaggagtttggcaggcatttaaacttcctgggtctaaacgtccttgtccatagaacaagggaattttattaaaataaatgacctcttctagctttaacagactcatagtctaggttgtatttaacaaagctctggtgctattcaagaaagaacaaccttggatttaagtagtgaccaaataacatttagtctcttttatcttatttacatagtttctgttggtccccagagataggagattccacccagttaaagtaagaggagccagattcttcgttttgttttgctttaagctggatttcaacccctcggggatggaatagaagcttagatatgaccaaatacatgcaggtcttttggactaaggagccaaagaagcacacgcttcatgaaaacagactgtcatttttctttgttaggatagacccactgacagttctcatctttacattctctcactctcctgactgtgaggtcaagtgagtatgtacatttttagatgccctttattaaattagaggggacaaataagaatctacaggtttctaaaccttcaccaaaagcctcttcccagcctattaccttttcaattaaagaagagagtgtataattggtgaatttgacatcacactcacccaccattagcattgttttgagtggggctggatttgagttgcatcattttcattccttttattggtaccccttagactgcctgctatgcatctgtgtgacagctggtcaaagttaggggtacagtcactagctgtgggaaagcttttctctttgaaactaattggtacatgtggggtttgaccttatgaccctggcctcattagtaccactaagctaataggctacagatattattaaagagaagcaatgtgtttttatattgaccccaaatgcttgttcctgtctttaaatagtaaaggaccagtttctgcaaccataggaccactaagaaaatgatgcaaaattagaaaataatttgcatttttttctgacatgctaatgcccttcaagaccatctccctcctgatggagagttataaacaggagagcgaagggttcaagaccaagggcatggcctctattgccaaacacttcaggtttgaaccccagctctgcaacttacttggtgagtgatcttgagcaagttatttaacctgtttgttcctcaattttctcatctgtaaaatggggcctgccttatagagttggtgttaggattaaattagttattatattggaatatgttaacttagaatattgcctggcacacagtaagcaccacatatgtgagctattacacttaattattctcttctgagatcaattgcagaatctattgccaatgctgaggtatatgaacggcccaattaattacacactcattttctctccttggcggcagctgattaaaaacctattatgagtagtaagaattatgtagtagtattagtaataatcacatgaagaatgttatgtttatctagtatatttactttttcaaaatccagttgtatcagctctctcactttgtcttgactccacttttggtgggcaaagattattgcttcatgggacaaatgagaaatgtgagacatgaacattttgtaacttaccccactgaatggttccagaaacaaagctaggttctcttgcctccaagaagtgtgtagcatagggagatgcttccaaaatcccagtagtcttctgggagataaggagcaagaatcccagggtgactaaattgattttgaacactttcttgaaaagaggcatgagagagatatctatgtaatgaccctatattttagcttctggaaaaattgaggaaattctaagaaaacttcctcaggatgcacaagaaaagtcttcttttagaagaggctaattagaggctgctgctgtacaagacccttcacagactgaattcagacagaacactgcaactcacctgtatacaccaggatatgcagtaggatctggggagcttctcttctgcccaacttcaaatcttcttcctcttcctgcaaacctcaagtgcttcattacttgcaactatttgtttaattggtctttctcaaagacctactaaaaaaatgctgtttaattggaccaaactttttcttggggtgctattagagtcgtagtaatgggaggtggtcacagacaccataatccaggcaggaagaaacttccccaaaataaaggagatgtgggagcagccagtggttgggatgcaagaccgccacctttgctggtattactcaacgagctgtttgaagaccattggtattgaaatgtatcctatctccagtggctggtaggggagacattttggatccaggagagaattttggcattctgctaattcaatgggcctccaatactttgaccagacatcttgtgtcgtgccatcattgcccaagatctccattagagctgacttcatctcttcagcagccttccttctggttttacagcccacaccacagattcagacacagtgaaaatagccacaaggtctctcccaaggagatgtctcattgttctcgataacagcctgcaagggctttgctcttttgtgcagccagacatctttgacaacccatggtggcaaaaaatcgttttcgcattgagctgccgccctggcagagttccatcgtacacctctccatgagacatcccaaagaattactttaggttgcctggaaagttcaaacccccaggccttattagctgttagactaaggagatgacaacacaacattttgtttgtctttatctatacatgagatcatgtttaaaacatagcaaagaggtgttatttgacaagcaattccctaactgcagtaagttgtatgcctatttaatgcattgtttagtctcacattggctaaagccacttaaagaagcacttggcaagctctagcttttcattcttggcatagctgcccctcttactttttcactgaaccttaaataccaagaaaaacccttcacttactttacatctggcatattgctttctgcaactggccagaacctttgcatgtggccctctgcatttgtcctaaggtaattaatggctttgcaatatttgatcatcagcagcacagacacaaagagaaattgtgcaaccagcagagaccaaacagcctcttttaaattaaaatcttttgcattaaatgctcaggcattctgcaggaataaagatttccatgccaaaaaaaagaggggtcaaaaagaggggaggaaatacccaagaacctgattattgattaagtatacatctctgtaatctctccctcttaaaactacccgattgttcaaagaaaataaaattgaagattgctacaaacttagaagtccttgaaaaataaacttgtcaatattatgcaatctagtcatgtatcaaaactgcacttttaccccatacattcatgtacatgaaaatgataataataagaattagccaaatgctgactgaaatccagagagaagtgacatgtgcaaagtcacacagctaattagtgacaaagggagaagatactgtgattctcttttctcatcccactgcagtattcctccatttagcaacaattagccatgtctgtgtcaaagttacctggtaaattaaaatgaccaagagaagccaccaatcaaattcatccaaatgagcatagatcttgttggttccttcctttcaataaattcacctgagacttaggttaacacttttgctccctcgctgtggaaatttgtacaaactaaagtgattccctgcctgtctcctatatggtgtgcaaagccttcacatgtactttgggcattccgtccacttaaggtcataatttgactattattgtatattatactattctgactctatggtcactctctccaatgtcaactttggtagaaatgctttggaatgacttagcaaagctgcgctcctggaaaatctgaaacagtctgacgagttgttggatcagctttttttttctggagatttctctgactgtcccaaaccaaaagaaatttacacctgatttttttgcagactcccaagcaaatactctatgaatctcttctggttttcttctttcatctctggtctttatgaggtactaaaaggctctttgagggacccttttaaactttgaaaatttgtcagacatagaagcaatccggagagtattgctgggaagatggtactacctctgtgccatttaccctgatgaaatgggtctttcatcagaggcaagtgatttagaaagtctgcactgagccaacccacatgcagccaactcctgattacctgtaaaactggggaaaggagcaccaagacttagaattctggattaaatggatttaaggacactatagatttttcttctccctccttgctgaacctatccccaacctctttatgagaccaagtgatgtgggttttttcattttggtttttgtttttttgagacagggtctcagtccattgctcaggctagagtgcagtggtgtgaacaaagctcactgcagcctcaacctcccaggctcaagtgatcctcctaccacctcagcctccccagtagctgggaccataggcgcatgccaccacagctggctaacttttgtatttttttgtagagatggggttttaccatgttgcccatgctggtctcaaattcctgggctcaagtgatctgcccgcctcagtctcccaaagtgctggatttacagtcatgagccactatgcccagactgttttaaaatcttaccttattctcatactcctcagtcaactttccccaagcgccattggcaacagagtttttcagcctcaacactattgacattttgggtcagactattttttgctgggagagggagctgccctgtgcattatacgataactagtaacatacttggcctctatccactaggtgccagcagtcccttcctcccagtcacgaccatcaagaatgtttgcagacattgccaaatgtacccctgggggcaaaatcacccccgagtcgagattcctagactagacaagtctccagaagagaggcaattagagagattggaaaaccatgagtgaaagtaacacatcaattctcagtcactgtttaatccagagcctatggtccattgcctggagcaagccatgagaagagtcattgtggttttgaaagcttttctctgaaaaataaaagttcttcctataggctatttaaaaccttttgcaggccttttctttcttgccttatagccatggaattcatgtattttagtaagagaaggaagggcaggcagaaatggttcagagtagaaccctgtgcttagaagtttgattgagaccacctggagcaaattaaaaagaagttggttatctttatttatttatttatttatttatttatttatttatttatttatttatttgaaacagagtcttgctctgttgcccaggctggagtacagtggtgcaatctcggctcactgcaacctctgtctcctaggttcaagcaattctcccgcctcagcctcccgagtagctgggatgacaggcatgcaccaccttgcccggctaatgtttgtatttttagtagagacggggtttcaccatgttggcctggttggtctcgaactcctgacctcaggtgatccgcctgccttggactcacaaagtgctgggattacaggcgtgagccaccgcacccagccagttgtctttgaccttaacatatgtggctgttcccttgtcaggcacataataaataaactcaattcctcacaagtgaaaaagcctggttcaaaggtaatgaccaaatttttctccttttttcttgtgcctttcctatctgagtcctatcacagtcattcaattgctcagaaacagtcagaagttccccatttgggcctgacccagctcccctactgaacagttctatgaccttggagaagtcacttaacgtccttgagccttacatagcttatcgtaaaatgggaataaaaatacacttctgagttgttctgaggctcagctgtagaagtgctttggaaacgaagggccatccaaatagtcacaactgatgaactgaattggatgctttttgccttgtatcccaaatcatggctcacaaattagatcaatgccatgatttctgtaaaaccttaggcaacaattataatctggaaaagttaatttggcagtgtatcttctcaacctcattaattaaaaaagaaaagcatgttgctgcctcctgttcacatatcactttacattgagaccacccaagagttaattaatgtatcttcagaatgctcactcagtatcactatttcctctcattggaagaagcagcaaagagatggggcctatcattttaccacactgagcttgcttaaagactttgttccccacgaggtgattgggtagtcactgagagatatcacatataggcaaaagatttctattctctcaagtcagctattggtgatttctgcttagcctcaaaaagcagcattcagcagaggaaatgtactcactaatcagaaaaagtatcttcgcactctcaagttgtacatccaaactgttttcttatcacagcctgacttaggggaattaaatttttttgtttgagaatggctttgatgaaatcaaaattgtgagttttatcaccatatggaccagtgaccctgtggttcttctatcccaggggcctcacatcccatggctcccagagacacaacatttgatgcagttgtaacacttttgaaccacacaatcaaatactggctactgctactgctgctactgtaaattttcattctctctattttatcttgagctttttcagaagaacacagactaagttttaagggttttcccccctcctgtttttcacagcatctagtatggagcacttaataaaatttcttgatttgatattgaaaatgtcattccaaaagctatttccttcttccagtccccagaatatataccatgacttacaatttgctactgacatcattttggatctgagattgaagatataggagaaaaaagtcaacaagtctacttgagatgtcacctcttaggaccccttcaccactgctccactgaaaatgcctagctcaagattaagctaactctagtcaacaggtaattttcaaaggctactggaccctcaccaataaggtctacttatgaaagtgagaacagaacctccactcagttcctctctacttctatgtgagttatttaaaaggagtagtgttgggcaagggaaggtatgcaacaactggtacatccctggctaaggtatgaatcacagctggagcaccagcccttagagaacactacaagtggtttctatttgccaatgcagaattgaaaaattagagagctagaaagaaccatatagagcaatctgatccagtgctctcacttcccagatgaaaggaccaaggtccagaaaaaacatgatgtgcatgaggtcacaaaagttggtcggaaaagctggagctactggtcggaaagccaaggctacatttcagtcacctcatgcttggtccagagctctttgcacagctggaccctttaaacttaacctcaataaactcatatgagggtagtgctcataaccacagtccatacactcaaagtgaactgctatgtcatattcttctggtgtaacacctcagaatagtttctagaaaattaagctcttagccattcatgaaatcttcatttactaatttctaccatccatcggataacatcatgaagtctctttatcccaaatctgatcgacctgactgtaggactgtgtaccttaataaaccctagtagtttgagggattggaggcttacaaaagaaataaaggaaaaagctgattttaatttgaagtcaagccagcatgttgcattgctctctaactcagaagtgaccccaaacaaaaggaattatcgatgtgttgtctgtcttcacacattcctaacttgctagtcttccacgaagatcttccatgaagaagcttaaattttcctacgtagtttgagttttatgtttctttccattaaagtttccagccgttcatagacatatttagtttggcagaaggtagtggtgactcacggaatgaagaaatttgaaggcaaagctactgaaaatggatctaatttctttttggggtaatttgggagcgagcaattgataatttgtctaattttcctggtttcatcagctgccacactgccaaatatatgttccatatggatgctttgttattcctgaaacatataacaaatttcagctgcgactttatctaatatggtcaaagggtaagactattgttggctcattttctcattgtttttggctggacaccctcaatcaatggctattgagtgaaaaaacaggatcatattgaaaccattttcagcatgttgcttcctatggatccaaccatttcactgtgagtttgtcaagcacagcaaatgttctctattaaagggatgactgtgttctcaacatggatgcgactgccagttaattgcattcaccttataattccagtctctcaatttggtgtagagatggttttggctctccaaagattccttcttttctgctgattcttacctcctagtatggaacagatatttgaggatgaattttgagaagatcaatgacacacatacaataagcatcatttcaggcttcccttaagttagacagtctgtgggccctgagtcatatgaatcataagacttgtgaaaggtattagtgccacaaatctggctggcatttctgtagattatcggtgctttgcatgcaattatgaaaatctctcttaccagataagtcagatgggggttcagtaacattgcacaaaagcatcatgatgtgtaagctaaagacaaagaaaggcttacttctggctggttctgaagtagacactggccacttagtgttttaaagtcattcagtggatgattggtttcaatgaatataaatgattttgatctccctccccaacacacatatcaaaaacaacagtaataataaagaacagaaaagtctatctcttagaaaaaccagatttgaaaactggctagggaaagtgttttgtggcaagaaacaatctcttgtatctcttgtttacaacgggctaattggtttgattaacaaattgtctaaggggaatgtatcaatgttgcttaggaaaaacaaagcttagtgtgtgaaaatacacatagacatagagtcttgctactcaaagaaacctgagggctttttagaaatgcagagtcaggggccctgaccccaaaccaattaaatcaaaatctgcattttaacccgatacccacaggatttggatgcgtgttaaggtttgagaagccccatctagagaatggcaggaagtaagaggtacttgctaattcgtggctcacatttgataagacagagtacaaaaagatcaactgtcactgcagatttcaaaaatgatgttaaggtcaaacctgcttgtggccattaacaatatatatgctgattcactggtatttggtccagattccctcaagttggtggaacctttcctcagaggaatgatagaactgctccaaggataaccccatggcctgaccttctgtgaactggaaattggaacttctaattgctgtagctgcaatttccaagcccatcactggggccaggaactgagccccagtccttaagatcttgccagtttctttttcaatagttacatttttcaagaacagccaagcaagagtgcttcaaaaaaagaaaaaaatctaattgcattaagtggacgaatgtgtgttttactcatatcaaatttcatcttttgttatgctcagccagattctaatattatttcttatgggtctgagttaattagttctatgagggttcccactgggattttagagaaatggttttaaggtaagaaaagtctattttggtcacctatcagaaggtgaagggtaaactttaaaactgcttttcaaagagatacagctggcttcgggtctttacgagggtcattctgaaactcaagttccccagcctgagcccattaatgaaggtcattaatgaaggccttgatatctgaagggtgtgcttcttcaaagggacattgtcccacagcagggcaggtaaagtgagagaggaccagtttttaacttgtatttcttgaaatgacaataaaaagaaaggggggcctttggattggagacttagaaaatcttggatttactcagaactctaccactaaataacttggtagatttgattattaatcccctgggtctcaaaacctgttgccattgaaacaatgatacgtaaatgaaccaagacattccacctttgaaatgtgataattgtgatctctcttcccagtttggcagggaaaaaaagtcactaatatagttttcacattgttttcacacatcttttacttgaactttaagaacatgccacttcttgtctatattcttgacaaggataagatagtattggacttagaccaaaactatatatttgatttgtttagatttaatgcatattattttaatacagtgatatccctgactcattggcactctacccattgaattaatgttgacaagataatctctaataccttttccagaatgtttactgctgttcatgaaaatctctaaaatcccttaagaaatctgacggatatggagattattgtttgtttctttgttagtattctctcattccttttgctgggtgctttagtaaaatgtatggaagtaaaattatagctctgcccaatttaattgtagaatcatcagctgggcacctgctctgtgtttagcaatacccaagggagaatgatgccatcccttcaaagaggtttctttgtggggggacccatgaaattccaatcatacaactgcaggcttgagtcagatgggaatagttctcccctatggtatcccagtccacaccatctattgagtgtcttctggttagccaaaactaatgatgaaagaaatgagtaaagcacagctctatgcacatggaattgatattctagagcaatgtcacaaaacgtttccactcccaccccacctgcatctagtccacagacatgttttgtttggtccacccaatatgtatttgttttaattttggaaatataaaaatcaagagattataagcaaaaacaaaagattaacaacttctcttatgaaaaaaatggtgcaatccagcatggctgagcctgcattcttgagtggcagtttttggtcacccttttatgtgaagtgtgtgcccttgcaggcttaatttattcattatatacctatctggctcagcatacattgacctatgccactcctgctccaaatatgcaagatctcgttggatattaactatacgcttgtctattttattacgttcaagacatatctattgcctttgttctatcacatagttaccagactaactgctgctggtttatataaataagacacaaacctcatctttatagaactgtatagccctccccaaacagagcaagggtacagctgagctagaatttaaaagacaatcaagtcattctggtgaaggaagaaggcagattttaaaaactgaacattcaaaaatttagacatcatcaaagggatatttcctcaagagcaagtggagtttttagccggtgaccttgtaaagcaggaaaggccaggttcccagccaatgcaatggatgggttgcacaatcaccctaagtgttgagcacagtggagaccatcaacccaactcttcagacaaaacaagctgtagccttacagtttgttttgcaccgagatccataacagctgttacctatttactggaagatccttccagtcccatcactatttagatcctcctcatcgcccaccaaaactattgcaacagcctcctccatgatcgtgctatgtgcaagactgcttgaggccagccgtcccacccatgagattaatctttccaaagcaattcttatcactttatcccttttctaaacaacatccagggaccattcatggctatgtaaattgagtgagtccaaactccccaatttgacagtaaggcattttacgtaaggtctctatctgactttcttaatgtatccctacagcagagctcacaaactggcaatctgtaagttgtatccagtcctcataagtgtttttgtgtagtgaatacagtgtttttactaaaatttgaattatctgtcagtatttactaaatgactctccacttcatattggcacatgctttctgctttaccaaaaaccccccaccactctctattgccttgctctggtctacttcacttgcctgcatgacttgctgtagtcattgagacattgacttgattgactcaccaagtttacaaatgaccccctacatattccaacatccgacctatgtgacttattaccacctattttattttcctggactttcctctcttcttctctcaaatgtcacctcctctgtaaaatcttctaccagatgtcaaccctctttcctctgagagagagaagcaataatattactcttacagcactttgtttttggtttttttttttttttttggtcagtgttcctaccatctgcttaactataagctctgcaaaggtgagggctatgttttattaatctctgtaagttcccacttactccctcatacactgcactatccatgtctgcatggaattaattgttattcgtttttatgtggccttacaggcctagattagttatgtgtgcctttaggaaggcagtaactcatttgaaatgtagacatggctgagtcggaaggcctccatggacttttcggtattttcaagtacagagtgagaagtgctgtatctaaaggaaactactaagggaatttaggtaaacagaacatgatcctttttgttggaagctggctgtgatctccagattaaaacactattgcatgcctcatgaggagcatcagcaaatcctttagtatgcacaacttgtgttagaattgcaggagtcagtctattcctgggggctgggctgagataatgaattgagtcaatgccaggtagagcacaaatacgctagaaaaaatgaaacagaaagaactccgtatgctgcattcaaaagtgtcaatttctaaagtttctgaataactagaaaggggctcgcattcctgtttgccctgcagaaggattgtcaacaagttacaagattatagaatcaaatctcaggcaccagaaatggatccaacttagtgaattttgactggaatgaccagttgcaaaatattgaccaagaagaagaaagtcattatttttcaatgtcattaaattcagcaatgttttatggataactgtgtacctagaattgtgctagaaaccaaggggaagctgaaacgaaaaagccagagcagaaaactaacgttcatgaacacccatcatgctcccaaaaagtaacatacaaatctgccttaaggttaccttcaatctggttaagaaaacaattcttacccatatgtcagaataatcatcataaacctatagaattaaagtaagtaataatgtataaaaataacatttttttctcttattcttcctggatatggcagatctcaaaatatgtttactgaatttccaaaagaatacaaaaaattttcatccttctctctatatatacagtcttgtgaaatcgtccactaaagtttaaaagtaggtggaatttacaccttcatgtactctctgtctgcctgggtccttggctaggtagaggcatggttgtgttctatagactgagagaggccagaaatactgggcaaacaagccgctttgactttgagttgatgaaaagcttaggaaaaaatccctccttttgtagagctgccaggccagtctttccaactcaagttcaatgatgcgtcaaaattctacctggagtcatgaggttcccctcccatcttatcctctgtatcccccggtccagataggatttgatgcagataacatgggcactgttggttgcttttcttttagcatgaggattcctcaaaacagagttttgttaataaattaagcagaatttgagtgaatttaagcagagtttgagaatttaagtgaatgtctgggtatatttgaataatatttattttgctatcagcatgacaacattggatatcaaaaagagttctgaatatgaggtgaggagacctggacaccctgaaaagccttgaattcgctggttggctgggttctgtcaggcaattctctcccttgtcaaatgggtacagtcagccctgcctgcctacccaaagcagtttttttagtaccaggagagataatacctgtggaattgccacaaaattcttagaaaatcaggacccgctaccagtttccagaggtaatttgagtgaagtgttgttagaattgagggagggctaggctagagtacatcctgaagttgaaagtgggttagccaagatgcattcatgccccttcctttggaacgtggggactgtcattcctttttgggtcatgtatgatttctgtggactggaattacaagggcaaatgtaggaattgctgtaatattggtcagtacttttcacattccgccatcccatgagtagggataagaggttaattaaatacatctctcgggcagcagaaatgatcaaggctgtccctggaacacagttgcttcaaaaagtaggcttttgtttcagggaagatctcccacagagaagcatggaacgccaaagagaactataaataggactatcatttatgtcaattataaatagatttgttcccttttgattagaagattaatgctagctgaaactctgtagttgagcagaaatgagggaaaaacttctgacaaaatattattacatgctaacagttagatttgataccttttcctccctgaagtattttctctgattttccctaaatgaagactttcttctcgtttgaatttatgtccagtttagtctttgaagtgcgtttgttcatacttcttagctcctcaggatctctagattagacaatacacacacacacacacacacacacacacacacacacacacacacaccttttttaaatggaaaaaattgaataagttgaatgtccgtgttaggaatttaaaagggggcaaaactttctcctcagatgctcctaagatggccactacgccctcctccaaaattctgctttctgtgagggagatgttttaccaaattaccatgtacatatggttccttagcaacccatattaacactgagcataatgaccaaagagataaaggaaaagatcagtagaggaagggccataagccatcgctgctgagcacctgcctgatatggagccatattttttcattctccaggaagcgatatccaatgggatcccaactctttacatatggttaaccaacttcttgtccttgattgtgttcatgacttgtctcttcccagggccagagagagaaatggcaacttcccatccaagctgactgccaggttcaacctgggaggagagattgcagggtgcctcagtcctctactggacatactagaatgctggtctcttccctgctaggatgacaggatggggaattttgcctgagtctgcccaacttgcctcatggaggcttttgatttcagctactgcaaactaagcattaaggaaaggggtgcaggtagagactggaagagagggtagatgcaatgagtggagacctctgtgtggaactcctcccccacttgaaatgccctattctggtttcgctgtgagatatttcaaaatgaacttaacttgtcaactgaaaatgtactgcctgacctgagatttctggaaaaacatgaatcagaggtttggaaagtctgggttctaactcataaagaaatatattgaatatttataaagatataacattcttagaactgtttctgtgtgtgacctctctcttccaagtggcattgctcaacagcccagctctcatcagaaggctgacattatgctgttgtgcaacttgcagccatagcaatgtcactttgttaactgcacttgctgtgcattcaactattgagaagccaaacagctttaagtttgagtcagaagttctaggcaggatcattcttgtgaaacctgaacccttagttcttctaaaaagtcaaaatacatttggatcttgcaaatcgagaatgtcagagccaatattttcctctttaatcaaaacccaggaatggcacggtgggtggctggggccatgtcctcccttcttccctttcccttcccccacacttccagccaagattctccaggcttgagagagctgatgtggaaaagcagctctggcccttgtgtgttcagccctgaacctctctgccaagcaccacattaatggtggacactaacgcattgtgatatagaggcatgagcctgaaatcaccaatttgtccccagactgataggtaaacagcaaataggaaagccttcttcttccctctcatccctgcattcccaaatatttggggctctatccaacagcctggatctgcagcaaacatttgcctctgactcaattgaccccttcatggcgaatcttacatccacattggataactctactggctcaatctatgaacaattttcaaaattgaggaaataaggcccttttccattcttaaataggctggcctaaatattcttgggagcttcatggagagtgtcatgccctggcttattcgcaaaggtggcaatggccttcagactcagctaagacaaacattggcacatgcaggtggccatcagatgttaatgtttatcttgttcctctgtattgctgttatctttcagggaaatctaggaatagcagaaagatgaatctttccttccccatggggcatgtacttccttctagggagtaggaagagaaaaccctctcattccctagacctcatgagttactctggagggttggtagtagccatggttaatggcaagcttcagaagggaagagcttccaaaaccttgccatgatgcctgtgaagtgctgtgagagtactcatgtttctctggaagtcacatcatgtgtttaaggtaggccttgtacagggaggattttgatagatggaaaaattctggacacagaaactaactcaatagaagagcacatagacagcaaagtaaggagtatgtttgcaggtgcatgagacatccagccagttgtttgggtgttaggctagtgcagagcagtgcaagataagcccagaaagataggttggcacatctatattgaagaattataatagctagcactttccaagctcttatatgcatgacactgttttaagcacttacatgtattaactcactgaatcaccaaacaactctgtgaaataggtactatcattatcttcactttacaagtgaggacactgagacacagagaagtacagaatgtgattaaaggcacatagattctaagtgcaacgaatccagggttttaactcaggcagcctactcaagcacgcatggtacctgacaaccttgacagcatgctctatagagcatacatttattctttgcttcttcttccatccccacatccctagccatggaaatgctgagtaacttttaaggccaaactcatctctttcctagcaatgcctcttccaacttcatcataagagaaacttatctttccctgttagaggcctctctttttttgtacatacagtaattttcccaaccttacttatattagagttaagaatgtgttcatctgtgaacttcttcagacaagaattggcatcatattcctctgtatgtcctcagaatctgggacagtgatttgcatgtagaaagtgctcaattaacattgcctgagtataattgaataccggaggccactaactgaggcctgaagaaaagtttcctaatcctaccatacctctttaagatcatctttccaaattcaaaacaaagcaaaacaaacaaacaaatgccaagctctatagggaactctcccactcctgaaaacctacatcctataaaaaacactttggaaaagcacaatgtagaccctcctaggccacacacctaacacattattccagctttagccttgcttgactgtaaaagaatgctaatggcagcagtgcttctaaaggcaaaagcacagccacacatgaatgatgtatctcactaagaactgtggggttccaggatttctgagaagactcctttcattctgataaagttctcagcttctcgacccccaaatcctaactccctgaggaagattcaaaaacacccaatttaattaaagcctttttttttctcatattgaagtcacaagttttcacagggtgcaacgtgttgctaacccagcaatttccagacaaggctgctcagcaaagcaggtgctgttttctttttagggcccatgtccttaaattgctttgctgctcagatgtctcaaaggtccaagtttctctggcagattccagcccaagcgctcctctttgacccagctctttctggctcagaagacttctttttcccataggtgcagttagccaataagaagagaatgtcttatctttttaaaaagtacacttccaatgtcttcattagttatcttcaaactcagagggtcaataatcacagcaagaggagcaccatgccttctggaaggatgtcaaataagcatgctgaatggcctacagttttgtaattggcaagacccccctaacacagattgagtgttacagatctgcaaagttcagtgctaaaatatgattgattagtgtgcaaatatttactgagcatctattttatgacaggctctataacagatgctagggatatgacatttgtatgagtccattctcacactgctataaagaactgtccaagactgggtaatttataaagaaaagaggtttaattgactcacagttccacatggcagcagaggcctcaggaaacttatcatggcagaagaagtagacatatctttttttttttttttttttttttttttttgagacggagtctcgctctgtcgcccaggctggagtgcagtggcgcgatctcggctcactgcaagctccgcctcccgggttcacgccattctcctgcctcagcctcccgagtagctgggactacaggcgcccgctaccacgcccggctaattttttgtatttttagtagagacggggtttcaccgtgttagccaggatggtcttgatcttctaacctcgtgatccgcccgcctcggcctcccaaagtgctgggattacaggcgtgagccaccgcgcccggccgaagtagacatatcttacatggtggcaggtgagagagtgtgaagagggaagagccccttctaaaaccattagatcgcatgagaactcactcaccatcatgagaacagcatgggggaaaccacccccatgctccaatcacctcccaccaggtccctccctccacacatgggaattacagttcaagatgagatttgggtggggacacagccaaaccctgtcaacactgaaccaaactgacagggttcctgcctcagatgcttagaggttagccagaaaggtgaacatggccagtgccaaacaatagcaaggaagggagagatccctgtgcactggaatcatctgagcggtgagctatacagtatattcctcaagttttctttccttcccaatggttggaatatctttctcatcgatctcccatcttcttttcactttctcttcttttcatcatctacagcatgcattgttacacacccactgttacaaaaactggtattagatttgttgtggcaagaaagcctcactgtccacaggcctacatcttagcaccttgatcttgagcttctagaagttctggcttcctggaagttcattacttactctgcgtaaacaaatgaaatagagagcaataaaatgcgctcagtaattctcacattatttgtttgagatatactgaaattatctacagaaaggagagcaatactctcttatccttgaagatcaaatgcatcaaagagttacaggttttgataaaaacttcattgatgaacttaattcctatccaggagtcaattggtctacgtagaagagtcctaagaatagaaaagctacactaggtgtgcagaaccattcttgaaattctgtatatgaaactgtgactcacttgatataactttagagtgtatgttttgacacctgcttattttcttaccagtagcctttctggttttccaaaatctttctccttgttatccactgacaccatcagactaacctactataacaggttaacagactaagcaactataactaatctactataaagatcttttaaagcttcttcaaggctgaacctctgtgactctgcattcctagatcccaaatcctatcttcaattaacagaggtttaggagaaatgacccaagatccttcctctgaaaacccgcccaaagtattattgaaatcttattcaaattggaaagaatgatttaaaactagagacagagatggaagtatattattgtattgacaaacagcatgtagatattgagaaaatgaaattaaatgtagcaagagaacttatttaagtctcccaaatgagtttgaaagaaaggcaaatgtgccctgtatctgtatttgccaacacagaaagagtaataataatcatctccagattagatgatgaaggccttagccttcagatggaaggcgaagaagagattgaaaaatctggaaatgcagcattttatcaagtcgaatttagtcccagagcaattgggcatccatttgtctccaagtggagcagtgtacgatggctgtttccattcgtggcagagagctgctggggccatcggagcaagagatgcttcacaaagaaagtggaaagcagcgccaaaaggcaaatacaattcctgtcacatccaaaatagttcatttagctctttatgctactctcttactctttgtcatggagcaatttctaggagagtcgcataaaagtagggagatttttagctttgagcagcaaataagtgaactgggcaaggagagcatgaaatttagtgaggaaaaagaaaaagaatgacaggggaataatataaaaaccctgggaagaaagcacttcccctgtagaaaatctactatatgaaaagtcactcctctaaaacagatatactcaggatggattttctccattcggaagagtccatcactttaataaaacacttttgaagtcttcatctcgtaacctcacgtttaacatgtctgagcacttcttgagaactcgccctctctctatgctactggatgagacgaaaggagaatccgtgagcccggtgttctttggaggcctctctacctacgcacatttgtgcttcacttctgccaagtgaatataatatgacaaggcttagaaagcccaggtgatccagagggcaagagtttgtgtcaagaatagaggtagaagaaccatcgaattatagggctgtaaaagaccttgaattcataaaatccctccacagagaaggggttaggattcaaccctagaggaatgtcagcacaggcctctctcaggatgtcccttctctgacatcctgagagaggcctgtgctgacattcctctagttggggggcgggatggggaacaaataactttactgccttcctgttctgcaaacagaaaaccgtagcaatggggtatcataccaatttttcattattattatactttaagttctgggaaacgtgcagaatgtgcaggtttcttacatagctatacatgtgccattgtggtttactgcacccatcaagccgtcacctacattaggtatttctcttaatgctatcccttcccttgcccccaccccccgactggccccactgtgtgatgttcccctccctgtgtccatgtgttctcattgttcaactcacacttatgagtgagaaaatgtggtgtttggttttccgttcctgtgttagtttgctgagaatggttttcagcttcatccatgtcccttcaaggaacacgaattcatcccttttcatggctgcatagtattctatggtgtatatgtgccacattttcttcttccagtctatcactgatgggcatttgggttggttccaagtctttcctattgtgaatagtgctgcaataaacatatatgtgcatgtgtctttatagtagaataatttataatcctttgggtatatacccagtaatgggattgctgggtcaaatggtatttctggttctagatccttgagaaatcgccacaccgtcttccacaatggttgaactaatttacactctcaccaacagtgaaaaagcatttctatttctccacatcctctccagcatctgttgtttcctgactttttaatgatcaccattctaactggcatgagatggtatctcattgtggttttgatttgcatttctctaatgaccagtgatgatgagcttttttttcatgtccgatggctgcatatacgtcttcttttgagaagtgtctgttcataccctttgcccactttctgatgagatttttttttttttttttttttttttttattatactctaagttttagggtacatgtgcacattgtgcaagttagttacatatgtatacatgtgccatgctggtacgctgcacccactaatgtgtcatctagcattaggtatatctcccaatgctatccctcccccctccccccaccccaccacagtccccagagtgtgatattccccttcctgtgtccatgtgatctcattgttcaattcccacctatgagtcagaatatgcggtgtttggttttttgttcttgcgatagtttactgagaatgatggtttccaatttcatccatgtccctacaaaggacatgaactcatcattttttatggctgcatagtattccatggtgtatgtgtgccacattttcttaatccagtctatcattgttggacatttgggttggttccaagtctttgctattgtgaatagtgccgcaataaacatacgtgtgcatgtgtctttatagcagcatgatttatagtcctttgggtatatacccagtaatgggatggctgggtcaaatggtatttctagttctagatccctgaggaatcgccacactgacttccacaatggttgactagtttacagtcccatgagattttttttttttgtaaatttgtttaagttccttatagattttggatattaccctttgtcagatggatagattgaaaaaattttctcccattctgtaggttgcctgttcactctgatgatagtttcttttgctgtgcagaagctctttagttcaattacatcccatttgtcagttttggcttttcttgccattgctattggtgttttagtcaggaagtgtttgtccatgcctatgtcctgaatggtattgcctaggttttcttctagagtttttatggttttaggtcttacattaaagtctttaatccatcttgagttaatttttgtataagatgtaaggaagcggtccagtttcaattttctgcatatggaaatgggctaaatgccctaattaaaggcacagactggcaaataggatagagtcaagacccattggtgctctgtattcaggacactcatctcacatgcaaagacgcacataggctcgaaataaagggttgaaggaatatttaccaagcaaataaaaagcaaaaacaagaagaggttgcaatcctagtctctgataaaacagactttaaaccaacaaagataaaaaaagacaaagaatgacataatggtaaagggatcaatgcaacaagaagagctaactatcctaaatatatatgcacccaatgcaggagcacccagattcataaagcaagtttttagagacctacaaagagacttagagtcccacacaataatgctgggagactttaactccccactgtcaatattaggcagatcaacgagacagaaaattagcaaggatattcaggacttgaactcagctctggatcaagcggacctgatagacatctacagaactctccaccgcaaatcaacagaatatacactcttctcagcaccatatcgcacttattctaaaattgaccacataattggaagtaaaacactccttagcaaatgaaaaagaatggaaatcataacaaacagtctctccaaccacagtgcaatcaaattagaactcaggatcaagaaactcactcaaaactgcacaagtacatggaaactgaacaacctgctcctgaatgaatactgggtaaataacaaaattaaggcagaaataaataagttcttagaaaccagtaagaaaaaaaagacaaaatgtaccagaatctctgggacacagctaaagcagtttttagagggaaatttacagcattaaatgcccacaggagaaagtgggaaagatctaaaatcgacacctgaacatcacaattaaaagaactagagaagcaagagcaaacaaattcgaaaactagcagaagacaagaaataactaagatcagagcagaactgaaagagatagagacatgagaaacccttcaaaaaattcaggaatctaggagctcattttttgaaagagcagcaaaatagatagaccgctagcaagactaataaagaagaaaagagagaagaatcaaatagatgcaataaaaaatgataaagggggtatcaccaccgatcccacagaaatacaaactaccatcagagaatactataaacacctctatgcaaagaaacaagaaaatctagaagaaatggataaattgctggacacatacaccctgccaagactaaaccaggaagaagttgaatccctgaatagaccaataacaagttctgaaattgaggcagtaattaatagcctaccaaccaaaaaaaaacccaggaccagatggatgcatagccgaattctaccagaggtacaaagaggagctggtaccattccgtctgaaactattccaaacaagagaaaaagagggactcctccctaactcatttaatgaggtcagcatcatcctgacaccataacttggcagagacacaacaaaaaaagaaaatttaaggccaatatccctgatgaacatcaatatgaaaattctcagtaaaatactggcaaactgaatccagcagcacatcaaaaagcttatccacgacgatcatccctgggatgcaaggctggttcaacataggcaaaccaataaacataatccatcacataaacagaaccaatgacaaaaaccacatgattatctcaagagatgcagaaaaggccttcaataaaattcaacagcccttcatgctaaaaactctcaataaactaggtattgatggaacatatctcaaaataataagagctgttcatgacaaacccacagccaatatcatactgagtgggcaaaagctggaagcactccctttgaaaaccagcacaggacaagaatgccccctctccccactcctattcaacacagcattggaagttctggccagggcaatcaggaaagagaaagaaatagttttcaaataggaagaaaggaagtcaaattgtctctgtttgcagatgacatgattgtatatttagaaaaccccatcatctcagccccaaatctccttaagctgataagcaacttcagcaaagtctcaggatgcaaaatcaatgtggaaaaatcacaagcattcttatacaccaataacagacaaacagagagccaaatcatgagtgaactccattcacaattgctacaaagagaataaaatacctaggaatccaacttacaagggatgtgaaggacctcttcaaggagaactacaaaccactgctcaaggtaatcagagaggacacaaacaaatggaaaaacattccatgctcatggataggaagaatcaatatcatgaaaattgccatactgcccaaagtaatttatagattcaatgctattcccatcaaggtaccattgactttcttcacagaattagaaaaaaccactttaaatttcatatggaaccaaaaaagagcctggatagccaagacaatcctaagcaaaaggaacaaagctggaggcatcacgctacctgacttcaaactatactacaagtctacggtaaccaaaacagcatggtactggtacctaaacagatatatcgaccaatggaacagaacagaggcctcagaaataacgctacacatctacaaccacctgaactttgacaaaccggacaaaaacaagcaatggggaaaggattccctatttaataaatgatgttggggtatcataccaatttatgagatggtcatttggctcttgagtggaggccacctctcagtgactcagcaaaatttgtcctctgcacggtccttgtatcttgtaccacctgaggcaatcaagtatattctgctttatatcatagttatttatgagttaacgcatctccttactgggtcccaaattccttgaaggttgcaactttgccttgcctattcattttcacatactacgcggcacttagggcagtgctttcaatgtagcaggtaaccaattgttttgcttgcttttaaagtagttgtttttaaattgacttgagcttagttctgcaagaagactagatgagatatgattttgtcatcataaatttaaaatgtccttaatattttcttaatatttaatctataaagagtattcctggcacatagtagacagtaaataactttgttgaatgaacttacgcgtaaatacatcaatgactgactgctcatggagggtgccccctttttgcccatgaagttgaggtggtattgggagaatgctttcatttcctgatcctaactctcctgttgcagtctagtattgtcaatacaaaggtgtcatcattaccgttaatcccaaactgatgtaatctgaacttttgctctgtgtaattacagatatggggtacatttcctagaaaagaactgccctggacaaaactgttaggtagaaaaaaaaaaaaggaaattgggtgtcttttttccatattgtcaagagcagagaggacctcactctgccccctccctcttgactcctcacacccaatagcagcaccaatgcctgccacagaattatgagcacttcagccgggcgtggtggctcatgcctgtaatctcagcactttgagaggctgaggcaggcggatcacttgaggtcaggagttcgagaccagcctggccaacatggtgaaaccccatctttactaaaatacaacaattagccagacatggtggcaggcacctgtaatctcagctatttaggaggttgaggcaggagaatcacttgaaccccgggaggcagaggttgcagttagccaagattgtgtatcattacattccagcctaggtgacagagtgagacgctgtctcaaaaaataaataaataaataaataaaaataaaaagttacaggcacctcaaggttgcctgttttttgcctactgttgcttcagttatgtactctcagtgatctgttaccttcctttccagaaactctgtgtaaatatatcttaattcaatgaacaaagccataatggagacattggaggcattagtagccagccagtgatctgttgttttgttcattcaagaggaggttatcatgaaaatactctgtgagaaacagagttccaggattacactggcttaaacaatatggaagtttgtttctcttatgtagtagtcccaagatacatagttcagggctgctgaaccatctgctgtgcaaagtagctctatgccacaaaatcttcaagacccagcaatttctagatttctatttcaccatcccagcatggtggccctcatctgcaggggccaagatggctgctggaggttcaacattatacccatgctctagccaataggaagatagaaaaagcaaaggatgcattttggttgtcttttaatgaaggatcccagaagctttcatgtgacactttcacttatatttcatttcctagaacttgaacacatagccacacagagttctaagttaggctggaaaatacagttagctgaaaattagggtttctattactgcagaaagagggaaaatagctattgggaaattactaggaatctctgccaaagacgctatccctgagctccttacgttctaccacttgagtgccccaaatcaagacactacaaaatgaaaggttgatctagtgtcagccagatctagatgccagtcctattagcatcacttaatgagctttgtgatctctgagccattttccatgctatcaaaaacagcaataacacctccctattaggttcctgtaggatgatataagataaccagtggtgcctagcacagtgcctggcatatatggtagatacttagtcaacatttatttttaccattctttctgcatataagactggctcagctccacagtttatatcaacttgggcacaaaaacattaacaaagttcctggcggccatatttcttgagcaattttatactctatccattagccagatgtgttagagcaaatgagcggctgctcaaaagaatgtctttatcctcacttgcctttttttcctgttattgttggatggtgccaaacaagacagaagcaattaataaacttgtcagtatcataagagatgacaattagaatcagcagagcttaaaaaggagcctgaaatcataggggttgaggtagtggggtttttaaaatgcaaagagcatgccttttcacttcactctgtttatcaggccaagcaaactaaagtgaaaagtcactgtggaaaaaggtgaagaaaaaggtacctattttgcatcacccactatgtcatatcaagtaaatgaattaaaatcccatgtcaagtactgggctttattccccatggtttcacaccaagtttctcttagaagatactctaagatgcactaagctttcccattcacttctgtctaggcaggaagattctactcaagttaagaagcagtgttgccagatgcagtggctcactcctgaaattccagcaatttgagaggccaaggcaggaaaattgcttgagctgaggagttcgagaccagcctgagcagcatagtgagaccctgtctctacaaataatcaaaaattaaccagacatggtaatgcatgcctgcagtcccagctactcagaaggctgaggtgggaggatctcttgagcctgagaggttgacgctgtagtgagccacaattgcaccactgcactccagcctgggcaacagtgcaaaaccctgtctcaaaaaaaaaaaagaagaagaagaagaagaagaagaagaagaagaagaagaaggagaagaagcagtgtaatagaatgacttagtgctcagaaagtcttgagtttggatcctggtttgggcatttaataagtgagtggcctgagacaaataaataaacttttctatggctcaatattttcaactgtaaagtgaagatgataacagtgtccatctcataggattgttgtaaaaggtaaatgatacagcatatgtataaaccagatgcactcaggttttccagtctccttgggtcacgcttttctccaatatctacataaaataacctgcaaccccttttcacattccatgggaagcccatcctctgcccacgagctctgaagatttggggtatcatgcttctctcaagttatttcactctatagcctgcttcccaaatatggcccactcccacttgtcattcctgaaaccattattctatgttccctgccccctttgcttcagctcatctaaagggcaggaaaacgctggaagcaggagagtcatgagtgcctgaaattttgcaggagcagcaggcagtaagagtgggaggacctgagatctatgatagtgaaagacatgatgcaatatattgcctgcctggttaaaggagtagaaaggagtcaaagatgactgtaacattttgagtctgagtaagcaagaagataatggtgcagataccagcaagtccagaggaggatttgactttgttgacttgactttggttgacattgactttggtcaatggcaaagattgactttggtttcagagacactgatgtataatagagcttttagaaaataattcccatgagtcactagggtgcctgtccaaagtgcagtgaagggactgcatagaagaagaggcttggagatttccttgtacaggggatagctgaagcatattccccaacacaatattaaatttgtaaattacctttaaaaatagaaagaaaattgccattttagagtctcaaaaggactgagcaccatctaattacatcacaaatgatctcccaaaaaactcattcctttagaccaggagatgagtttctattcattattatttttgtagctaaaatttcaaatcacagaatcataatatattgcagtttgtttaagccttaaaatgttcctggcccaaatgcctcacttaaatacggggaaactgacatgctgaagagtctgagtgacctatccttgttcactcaagttgaataatgctgccagaaattctactgttagaatttctgctctcccttgctcattttttcacctgcagtccagctaccaaagataggattttagacattctatctacatttaaaatgttccttctaactgcagcggaaacacaggtagcaaggagacagtgtagaataaggagccagaggcctggattttagtcccgtggaaaaagaaccagccgcactacctggagagaatcatttggtcttccaagttctcagtttcctgatttgagtagcaaggagatgggtctggattcgctctgaaatgtcttgcagctctgacattccatgattcttggagaaatcggctatctccaactgaatccacatcttccaatcccacacaagatggagaagcaggcccatcccagtatttggggaggcccatcaggaccaaagctgtcctctctctgtgggggctggagaagggaagcagctgaggcaacacaaccagtttccttcccattcagaagagaacaattgagctcctcccaggggaagccatgcggatttggacagtgtggggtctgtcttatgacaggaatggaggatttctgtagaatggggatgcttagcggccgtaagaatcaaagatggaaggaaagccaagaggtccttaggtccggtcccttgcctctagctaagccttccaaggcagatggttgtcatcctacatttaaagatctccagagtagcaattgctcagcttcccacagcgcctggggtcagcaagtttccccttataatcaagaaattcttcccgatgtttaagtggaaatgcttccaatgtggcccgagtctgtttacccttgtctagtcctcagaagagatgaggaataatcttctgtgtaataatcactcttcttatgcctggtgctggtgattaagtcacttcttagccttttcttttttgggctagatcttaacttcattttccacccagttaatctttttttttttttttccttctcctagctggatcttttccaggttctccaaatgagccttaaaataggagaatgaaatctgagttcccactctaattaaaacctcagcaatgccaagttttgtggaaagattactctctaggctttcgtttcataattctctacatacacccgcctcatattacaatttgttgtagtactttcctgctcactcagggcccacagctattttgttatagtacttgtctctgccagatggagcccatttttgtctattttaaacatctttctgtttttaatgacttctcgtttctaaattgttaaaggtctccttaaattcaaatcctaccttctaatgtttctttacctgacttagtgccatctgaaaaacattctagtccattactcatcattgaggaaggaaactcaattttgtatcaatccgagaagggcctgaatagcactcatttctcccaatactcacaagcatgctttgagcatgaaaaaaaatgcaggccaccccttctccattcatttattcatgaaacatcttttgaacagggtagacactggacacacagtgatgaacagtttctgtatccattcactaatgtgttcctacatgtgtacaaaatgtactccttaagcacttactctgtgtaaaaccgttgcaagtgttgtggctacagcagtgaatagggaataaaaggctactggcttcatagatcccacattccaatgggtatgggggaggacagagaaaaattacaagtcaagcaacaaataaacaagaccatgtcaaatttgataaacgttttgaaagaaataataggatgatgaacctgaggatggtactgcctttgatggattggatgggggaggtccctctcagagggagaccctgaagacctttcagtttagcagagttatgttattctgaaccaatcccataaagcaaacccacttccctcatgttttcttctgacagacccagcactaactttttctggggtgttcacggtttataatgccttgttactcagtaattgtttacattcccaaaggactctctcatgttctttaattgaaagaagagataaaagaaggaatttaagtatacaggcgcctttgttcctcaggaagttaatggacattcctggaaaatgagagtccggtggtctattgtgtggagaaagtggacttcagtaccatgaaacagatttcttcactgtggaacttgaataggctgttcctcgaccatcttgaataggctggtgaactttaagactctgcaaaataagataacgtttacagccgtttcctaagctttttgcacggaacttcttgaacaactctttggtgaatgctgagctgaaaagatctcttggatccttaggtgtcctttgacataggatttggcatttgggatgtgattccaaatcaccagcagccaatgtgcaggccagtcgtcgatgtctctgaagagtgtgatagaaagaagaccgcagaggaaggcagggagtccttcctgactttgcctcagcttctcttgtgaccctggtcacgtgtgaactatgcttctttagttttctcagatcatggaatcttctgagaatcttctgagtgtctaaatgtcctctttccaggaaaatccacaaacatacataattatacatcatgcagagggttgatatgcctcctaccaaaatgcatctctgaactggaggatttctaaggatctgtctgtccagtgtgaaagttcaaataggtttctgctctggtcatattgacatcttggtgttttggtttgtatgcttgcttttaattgacacataataattgtaaataattatggggtacacatgatgttctgatacatgcatacattgtgtaaaagtctaacagggtatttagcatatcgatcacctcatgcctttatcatttctttgtggtgagaacattcaatatcctcttttttagctattttgaaatatacaatacgatattgttaaccagagtcaccctattgtgcaatagaacaccagaaattattcctcttgtctaactgtaactttgtacccattgaacagtttctatctccccacctaactaatgctccgcagcctctagtgaccactattctactctctacttctattagattaacttatttagattccatatacaagtgagatcatatagtatttgtcttttgacattagtttggacaagaccccaaaagcaaaagcaacaaaagcaaaaatagacaaatgggattacatcaaactaaaaagcttctgcacagcaaaggaacaattaacagagtgaaaaatcaatcttggtccatgtttttgattagcttgtgtgctccttgggacaaaaccaagcatcctaccacctctcaagcttccttcagccttcaccacacaagtggtgctaatatgtgtttctgcacctgtgccaggttcctctgcagagtcccgaggtggccgttgcatgagagactgctagggaggccagggagacccctccacacagaggtaccagccagccctggggatgccccgttgacttctcaatgagtgaatgaggtcatttttcctctgtcttttccagaaatagattcgttgagaaggcatatgctgaaatgacctagcaaggcagcaggaaggaagtgactagaaagagaatcattaaaaacccaacttcttatttaaaggaaaatggaaacaggggcctggaggaatgctctaattgggaagccagcagtggcccctgccttccctcccagtgctcccaatatggatttcctgcctttgggcactgttggatttaaagctatgaaagcctcatacacagtctcatcagaagggctcagaaaagaaacagagcttcatgtgccttcttcctgccacttatgtatggaatcagttggattaattaaccctgtttcctcagtgagattgattctatcactcgctcgagaaattctgatgattaggtgtaacctaactgataaataaaaaattataattttttttttgcaaaattaaatgtcctctgctttgtataattactgtggttctatgggcaatcatcagtggtatattcccttccaccctcctttgccacaaattttgaatgatccatgaaaagatattttgatgatgtggtactgaaggtgccccaaacccacactgcctttttggcttctctccattaccctaggaggttatagggcaaaggcataggaaggataggtttgggttcctgggaagtctgtcttctgagctgaattttacaattgagcaagttggcttgactggccatctgacaatctggaaagcttaaaaataaattgaactcagtatttagaaaaggcagctttcaatatcagaagactagcttgtgtttctcgctttccatttggcgggggaatctgtgaaggctctaatttcctttcctgagacctggcttgttgtcttcaggtgctagctggaattcaaaaggacacagactgtgcaaataaaaaactcagggaagggaaaaaggtcaacatgtgttttttgtttcagttcagtttgttactttgaagggttataatattttttccttctgttttgcaataaaactggttctgctactagactgacatatagatacctttattaggcaattatcacaagagatatgaaggctttctgtgtaagaaagtataagaggtctttcccaggactgcagttcttcccttctctccagtgccacaggacgtgatcgtatattatatacacacatattaggagttgatattgcacacaggcactggcatcatttgagaacttatggaaaatgcaagtgcttgagcaccaccccagtacctgttgaaacagcaactctaaaagtggggcctgttgatcgtgttttaataagccatccaggtgataaacatgcaggctcaagtttgaaaaccactagtatgtgttagttatcttgggcactacctgcttcaggaacccccacagaattatgggcctgaatttcatcctttcatgaggtttggggacaagtatgtgggtggagaagtcttgccatttgaaatcaaaggaacttccagattcataagccacttaaactctcagttttttcatttataaaagtgagaataattccattggcaccataaggttgttgaaatggctgatgcacaaaaggtatgtgaaaatgatttttgaactgccaggcactctctggaattagctatatggattgatgatgccaatacacattatggaccacaggcatgctgggggatgtggggagactgtgtaacctgcgtatcctgtgtcctcttctatattctgagcaccaagcccaggcagataatggccattcactgagtctttgctgagtaattttaactaatattgggtaagtgttcactcatgcatgcatcacaagcaccttgtgaggtgagtatgtgcctctttttggatgcaacctgagaggttaaggagcttcctcacatcacatagttagaaatggtatgctcaaggctcttctttaaaatggcatggtattaatacttgcatgtaacctatgcacttttcttgtgcactttaaaatcatctctggatcacttataacatctaatataatgtaaattctatgtaaacacctgttatactgtattttaaaaatgtgtattatttttaattgttatattgttatttttaattttcgttcaaatattttctatccatggttgtggacatgaaacctgcaaatacagagaagacatttatgcagccaaaaaatacatgaaaaaatgctcatcatcactggccatcagagaaatgcaaatcaaaaccactatgagataccatctcacaccagttagaatggcaatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattacttatatacccaaatgattataaatcatgctgctataaagacacatgcacacgtatgtttattgcggcattattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaaccatcattctcagtaaactatcgcagggacagaaaaccaaacaccgcatgttctcattcataggtgggaattgaacaatgagatcacatggtcacaggaaggggaacatcacactctggggactgttgtggggtggggggaggggggagggatagcattgggagatatacctaatgctagatgacgagttagtgggtgcagcgcaccagcatggcacatgtatacatatgtaactaacctgcacaatgtgcacatgtaccctaaaacttaaagtataataaaaaaaaaaaaaaagagatgactgtacttttgaaatgccctctgaactcaaatgatgaaacaagtgaaatcaaaaaatttgtttttcaagtaaaaaaagaaatggtttagccagcatctgaacccaggcaatctcctccagagcctgtgttctaagcacttcaccacacaacgtcacaatcagttcttcataatgatgctaaggagggtggggtgagatgacattctgggagtgcttgctctgtgccacaagctgtctcaagctctctttgtgccttaactcacaaaaaccctgtagagggaaatgatgatcatcctcttttaacaagaaaggaaatggagacttgagactggccctggtgttaggtttcttctttccaaagtacaaatttcttctattgcttctctctttgtaccgtttgtgcacccaacttgtcaatccaccaatcagtgagcatttgtgaagtgtccaaatgcccagtgtagtgcttctaaacacagaaggatgagatgacagtgtctgccctcgagatgctcaaaatcaaataggaaagaataggccagtcttcacaaaacagaaaaaaaaggctaatatacagttaagagcttgtttatgatacataaagtaatcattttccatatttagaaggttaatgggaatgaggtcatcacacaagaattctcatgggggcctgaagaaattttgtgttgctagactgagacttcaacaatttctctgagcaattatccttgaactgttgagttcagcatccctgcattgcagtttcctttaagtttgttacagttaccctgattataaggagatagtgacctgttatcctgtttttttctgtgttgttaacttcttatccctggtactgagaatagcgtctacaattcagtaggtgctcaagtttacatgtgaccaaatgtttataaaaaggagaataataatgtggtgaaagaaggacaggagtgttcagccaaattttggattggatgagaaggcagattgtcagggatatcatgacccaaacaaaggtggagagacaccagattagggaggagccaggttctccagagaggacctgagttcagaaggagcacaggagcagagcctttgttatttaggatgcaaactctcccactcccatacactgaagtgtggtgctgtcttggcagctgtgtgttgaacctctctccctaccccagagaagaaacctcatacgtacttggtgattttctttttaagttttttatttttatttatttgtttatgtatttatgtatgtatgtatgtatgtatgtatgtatttttattatactttaagttttagggtacatgtgcacaacgtgcaggttagttacatatgtatacaagtacttggtgattttcaaagttgagagaccagagtcccttgtcatcaatttcattttccttggttttacttctacttagaaaacctttggatgccaaactcaagtcttggttacgccccatgtttccttcaaaggaaactgcactagtaattgaaaacagaaattggttcatttattctaactcacaggacacaggtaagatccctgtctgtgctccaccttggctgtctcttttctaatcccgggagaaaatctggatataattctctgctttgttgattaaattcctctggtccttatgcaaaattgtgggctttcatcaaaataggattgtctgataataccctttgactcagtaattccattccttggaatttattcttataaatgatctatgagtagtagaatgttatgtgtacaaagacatttattgctgcattagttaaaatgatgataaaactagaaatagcctaaatgcctagtaataggaaaatggttacagcaacaaatgatggcatatctactcagtaaaatattctgcagttttaaaattataattatgaaaactatatagcaatatgggaaacgcttacaatgtcatgttaagtgggaaaaaatacagtactgcattaaatttaggttatggctgtagctatgccaaaataatgtatgtatctggatgagaaacagaaacaattttgaaaaatgaagataaagtgatttgctgggatatcaagattatgggtaaaaacttctctatttcagtgtctgctaaagttgttataactttatgcaattcaagggtcttggcaggctatgaaacaaggctgactgccccatctgaaactgaaagggtcacaatgcactgggtaagatgtgagcatgcaagagctatgtcctaaactgcggaagtgcacagacttctctctcaagggaaaaagccatgcatggcattccttctggcattctctagaatacctaggataaaactctgtgcaactccatgtactcaaatcctggggttagtgggtccatttagatgccttcacccatattcttcattgatattagcatcatgtgcccacaagaatcccagtaaaggtatcaacttcagagttcttcatcaccagggaattctctcactttcaaggtgttcccgagtaaggtgacctggctttgcagcttactagatgtatgatttaggtcaaggtacttaaccttgctaagcctcagctttctaaaatgtaaaatggatgtagtaataatgctgcctccatgtagatactgcacatgtgtaaataagacaagaaaagtaatatttactatagtagcttagtaggaagcactcaataaagggaagctattggtagtgtattattgcaaacgccctgatttttctaaatgtcaaggagaaacttgtgtttaatggaatagttttcaacttcatttgattatatctttgggtcttgttatagtgccttctctctcagtagctaattagtatcctctaagtttgagtcttttcaagcgttcacgcttccatggaagtgttttctgcactgtgtttaaatcacgttagagaacttaactaagcttcaagcctagtagtgtggtatgtgtagttattctgacattcagagaaatgcagaagttcaggccagatagaaggctaagagaaagagaccccttggtgatgccacagatgactaacccagatgggggagatgagaaggctggggagctaagagccacagccagacacaagagaggaatttggtttgaaaagagagtattaagacatcaacagcctggaaagagaagtacaggctgatttgtgacctgttgagcaccaagaaaaaagatgtggcaaaagaaggcacagatgttctgaaaaatgcaaagaatttgtgatggctcagagtccaactgagggaaatgactgccaaaggcagatctcagtggaccgtgggagcactggctcgatgagaagacatgaggaggctaggacttcataactgacctttaccagagtgtcttcctgacccagagcaaggaagagagatgaagaggagaagatgaacaagtcgtttgagaagaaaaataaattagcaagtctttagaagtagattaaaatattcctgaaccagcagcctgctttttactaaaattcttttttttaagtcatcttcaatggcttgaacttacagttcagttaagaggatgtgtgtagctctgtctacattctctcatcagaactcctactactcagagtcaaaagcttcacttctttccttctatccttatcatatggaagctgctacaataccttgagggtcatttcattcgtcagcctaaaggttactaaatgtagcttttatataaacaggcatccagttaacatagtggagattacaaagaccatacccagcaggaggtcagtggattttgatattctcctttttcccaaagggaagaggcttcccattggggttacaaagtagcaggtacttggctggaaggctgagaagagagaatggaggctgccctgttgaaagactgtgtatcagaaaggaatgtttaaatcctgaacttaggccctcacttctgcttgagcagagcagagctacttatgcctaagtattggcataagtgatgcctccacacagctttctcaaacccagatgtgagacaaagagagcagacagaaggcaagtgtccagggaaataaaatggcccacaagcagttatcctggcccagtgtccctgcaccaaaggagcttccccagctgtgtgtcacattaggtctcacattgcgtattctcaaggaggccaactaattctgtaaccatggataggtcctgtgataaattggaaaatgacttctggggcaacagtctctagttctcagaaaggaaattcacagaagtgagcggttggccagaaggccaaaaggccattgcctcagagtggatcccaaagcagagtgatgtggccattcacagcatgtttgagtatgtaagaaaaaagaaaaataagaaagggtatgatttctcctgccaatatcataagctgagaatatttttctttgctgaattcagtaaaataaatcagagatttttctcgatggataattacatttgttaaaaagcatttaaggaggtttgggaatctgggcatgatgttggtcccagatgaaataaagatgagaacatgatttagcttggagacaaactgtaacttaaaccacacctctctttttctttttccttattcatagttcatttcttcaattcagaaaaatagttttcatgggggaggggattgggaggagtgcaatggctaaaaaacaggctcagtgaaggccagaagattactaagaaacctcttcagttgaccttgagtttgctgcaatatccaaagatatatggcattctaaaatacaataatttaaggaaggcatttacagtttcatggtcatgtttaagcaataatatattaagaataaaagggaaagaaataatagcagcaaaatcctgcagaaagaaaagaaataatggtctgatgagtccagattttatctgaaattcctgaaataatgttatcacaccctaaatgaaccattcagcctcagtctgcatacttgcttggtattttgaaaaaaaataatataataccacaacatgcttgagcactcttcgtatagacttttaaattttactgaacttagcagtaatttattttaccaaaaaaagaggatggcatttgaaaaactatgtttaatctttatcttctgattttatattctgggcagatgttgggtaccaatggcatagatgatgtcctgtctggaagcccattcatactatcagagataacatgtagttacggctagacatttccaaggaaactatgcatctgtccacctgaaaataagtgaatatcacacagagaactcatgtagcattgacagacaccaaggaaattagtgaaagaaaacattggcctatgttaaacactaggaaataaagcaatcttgtcaacaatattaacagtcctcctggggccgaaagagtcatttgtttaccttatgaagatgcggctttgtgaagaataaaagtaaaaggccatgcccaaggattattagaaagactcttctcatactaattctcatttctgagtgcccacagaagtgagggccatctctctttccttaggactccggttttcaaaatgtagtttgattctatcacctgcttcaaatgactctctaaagggctctttgtgccctaccttgagctggcttctgcaatccatatggatgggcttgtctccttcccctaatcaagcccaactccaaattagacattttccttcatcagactcaacactgatccctgctttccttaaattcagcttatacatcttgtttacatccaaaattaaatctaaaaaagagaaaactcttggttttctcatcaaacttctcttcactactatactcccctattattagctagaaacattaatatgccatatatatatatacacattattgattacaaagtgggtcgttcatgaccatctggatatttagggtcatctttttagaaggggtgcttgatattatctagcacatttggttgtttacatgagaaactgaatctcagagaatttgggtgacttgtgcatccacacagaggatttggggctgtaatccaatgttgttttcaatataccatattgccttgtttttattccctctctgcatatggcactgtgctaggtgctatagaaacattccccaggacctctcaatccaagaacaacactgttgatacaccagcatgaaagggacatcacacacaacccaactggcactcaagtctgcaccatgcagttatttatatcatgtaacaccaaaccatctcctcccactcgccctccctccttccctctctcctgtgttcaatctgtcgctgactctggctgctcccttctctgcgacagtgccaagagctgtttctgctgctgtagaggctgtcttccaacagtttactccctgatgcaggcccggatgcctctctttaaggagcgtccacctcccaacactctccctccagacacgccagactgccctggcagccgccaccccttcacctgccatcctggccatgtcactcccacccctgggacaaggtccagatgtgccctgagcaaaaaacaggtccctgagtgaggagactaatggggtgcagggcccctgagaacagagggcaggtgccaagttacaaagaaaaaaagaaatgaaagactttagagagaaactaaaaataaagagaatgtgttttaatatccctttggctgattcatctttctgtggcaccagaaagagggcgttaccctcgactcacgaatgccttatgttttgttacaaggcctctcttcccacatctgaaagagtaaaataacaataagagtgtaagtacaggagataatgctggtggcatagctacaatctccaagctggagccagtctcagcttacagttgtgtcagtctttctaaaaaaaagaaaaaaaaatgagacagctaaggcccacgtatgcgattcctcaacatcccttcttattctttctctggcagttttcatcttattcaggcatgggtccaagatggagtagtgattaagcggcctggtgacactgtatactgggtaactgcttattcccaaagcatggctggcaggagagggagccctggttttcagtcattccagggaggaaatgaaatgctaatacctagcctaccgtaaactccacagtggcataagccttgctcaaaagtacatcagtgaataaaaagaatgtcaaaggctgtcagtagcagaagaggaaaggaaataggaaaagaagagaacagaatgagatgagacatgaagtagagagagatgagagaaatactatgctgggtgaaagtgattgggaaaccatttctgaccatgtatttcaccacactcatcccaggccttgcagcattgttttgaaataagaagcattgatattaataccaaaaagccaatgattttaaccagtggattatttgggacaaatcaaaggtaacaaggggtcaagataagcccatacgccttatttgacttgattgaaagattcattctcatagtgtctattctagattaaaactattcagaccagtctatacaacataaagtttggacattctttcaagagaacagcctttcatttactttagtgaataacaatatctagaaaggccatctgcttcgaccaaactttggaaaggatgcatttttaagattaatggaggaagaggatacctgcccaactagctatgttaaccaattcaacaccataataataattgtcaacatttatatagtttaccatataccagccattgtgcaaaacactttgcatacattctcacttaatcttcatgccagtcctgggaggtactcttatatatcctttttacatatgaggacctgaatcttagaaggttgagtcctttgccaaatgaaagcttaacagtaaagaaacagtagaatccagactgtctggttccaaaacttgtgcttttagccacttcacatattgcttctgtcattggtatgacagatgtcacaggcagatcatactcaaattctgtaatatgttctaaataatacaaacaatataactagaaaatggtagctaacttatatagcaggccccactgaaataattcaaacataaaaatgacctcttggcatttgattatctttcaccttagacgttgataaaccaaaccattcccttaggacaatatttttctgtccagcaaagcatttctcttgccaaagtataattttgttggctacatctatctcctccaagccctggaatcagacctaagctagtaaattgtgctgtcctggcaagacactcatcctccagggcattgtatcaggctaaagtaatacatcatgttgcttcccactctgtctgcccatttctccaaagagtcctaaaacagtaatgtttctgctttgaatctcttgctgttatcgcccctaggacttgcactttagcttcctggccttggaaaccaaggtgagctgaatagcattcttttcacacctgctcttgacttgggcttccaaaagaaaggaggctttcaacaaaggggcagccccatagaactctgcttcattccttttagactcagagaaacaaaaacattgacttgattgggtaacggaagactttggtcttttgggttatggggatttagtagctgagaaagagttgctgagtagtgagggtgggggcaggtcgtagagggccataattcctagaaaaagatgaccttgtccaattgagaggtagtgagaccagcaaacactggcaattaaaaattaacacaggccaaaagtgaaatcgatgcctaaatttgataggttttcaagaaaagtttatgacatgcagttcatagaaaagagaacattttctcagtgttcacttttaagatgccaaaggtcaatcttacttggaaagatccttccacagaaaagcagaaatgtacagggtctcctagcaaagtataaggcaggcaatagatatattagaaaatcttttttaaaaatcctaatttcaaaatgttttacagcagctttggtactacagtaattcacttaattccctaatatgtgtttgagtatatgtgtgtacatactcatgttattctaaacataaaaagatgtggccagttgacagtagtaaaagcttctttccatatctttactaagcacatgacaatgtataactcaataattgctttaattcaagttgtgatttcaatagacaaaaatttaaaggcaaaatattgactctcaaaaggttttcaagtaaatgcaaatcctatgaattctggaaatatcaaccattcaatgtattacattggggtcttggtaataatataatcacaattttccttatattagaaatttatgaaaggactcaagtcttccttataaagccatcagagatgtaggaaatattgggaggtgattctcccttttccacaaatgaaaatagtaattaaaatgctaaaatgaagactactttaagattattcatatcttatacaattttcctactagtatctcatgcaaaataatgcaaaccaaatgaatttatattaaataaggcggcccagggagaaaatcttgccctaaaaaactaaccgttccaagttattctaaattggcaatgtgtctggagttctgaaataaataactggtgtgaaaaacctaagtttccagattcttcagatcttaactgtaatatttggttgtgtaatcaatccttagttaatctaagtgaaatgcaggttcatttttattagctaataatttcatttgtgttttttatcagacatataattgaacattttctagaaatagcccttaaaatccaggttaattgtcttacctcttttttcctcctttcaaaaaaaaaaaaaaagggatcaactttggaacctcttcctgttagcaaatttctaaagaatagttttattccttagaactcaaagaataacaaatgtatatcttaagaactagttgcttcccaacttgaaaacgtttgcgtttttttattatttaaatactgactcttcaaatcaatgtcttccctttccaatgatagagttgaaatagttaaaataaaactttctcaagaagcaagatgggaagtttcaaaggaaattaaagctgaatgcattgttccaactgcattcccagaaatctataagaaacacaaacttatgcagcatcagtagaggaggtagtaccatttttgccagtgatgaacaaccagagtcctttgttctagaagtgatgaccccggttaaatctcagcattacacatttcccttcagagatggaggggagcgagttgcttgcatccattacaaaatgtgagactcagctaattggtgtggttttgatgatgccacagttctgcccaaaacccttgtaactagcaagtttgccctgaagattattgaattactccccgaccatgcattatttaaagggaagtgctgttattatagaagccagctgtttaaccattggaattctgtctgttggccaacttttaagatacagcagtacattgtgactaaacattatgtctagtcatggaggagtcaaagtctaggataggtggtggccaacttgtctggtaagcaatctttggtctacatgctatgaaacctttgaagcaaaactgacaaaataaatatataaatttttaatgtcaagactttgtcagcaagaaaaattatgtggaatgttatacatgcctattcattctaacagttgagtaagtgcatgaacacttcctcatgccagtacaacagaactgttggaccacaccttgctgtagaagggactataccaggcaccggtctttttttttttttttttttttttttttttcgtagcatcgagtaaggacaattaaaaagtgtttagagcatcttggggaacttcaaatagatagaaaacaacttttcaccttctaagttagaatatatttgcacctcttccttgaagtggtttccaaccatactgaatattgggcccaagccagttttctccttcatctcttcacttgttatagaatgtctggaatagaaaagaccatagcagtcatccactgtgttcaacataggaatcttctgctaggatctctgacagatgtttattacagcctttacttaactgtttcctgcaatggggagttcattacttttcattgaaatctgtcccatatgtaacacttgtaaattcaaattgttaggaatgcttttcttatattaagccaatatttgtttttcattcacctaccagctctgattctgcccactgggacagaattatacagcacctaatgaagagtttctcaaggcaaagataaaacaattagttccatctcatttaaaaattagtcgaaatgacaatagttataatttagaagtcattaaaaataactatctcaaataggaaaatgtcaaatactgaagccaattgttttattagctttctaataaattacattgtatcaggcctctacctgtagaccatagcagcaaatatctttattgtggttctggaaaaataaacaatatgtagcatttgaagatatggaacaaaactttgaccctcaactgtgatctttcaattggcgagaaatgagttttttgtttgttaaatattttgatgtagagccagaagaaatcaagcttacagggcacttcaaaagatgaattaaacttcaaaaggcaataggtaaacacttgttatattcatatattcccaatgacgtacaatgagtccattgtagacattatgagtagacaaagaaaaacatctctctaggattgtggtagctagagataattggtaattatatttatggcaaaatcgaagtctgtataccactatttcaacaatattttttaaaaaaacaaatgtagacataagataaactttctctatggaacattgcagaaaagtcctaaaattctctgtacctaaacccatcgatctacagactaaaaagcatgagctcagagtttagatgagcttagagtttgactgagtcatgtcaaattctgagctcatttaagcaaggtgataaaccagatgcctggtatttcctgttattttttttgctgtgatgatgatgactttctattttacaagaggaaatcgacatgtggtgagtttcatggttgtttacagtcaccaagaagtgaatatacaatcatagaaactctctctgccatcccaagatgaaggttaatgtcttcctcttatagcagacaaggagctccagcacctgtgggacctcagaagcagaattaagttagaggagcttgaaagggaaatactaaataggtcctggaatcccaaagcctgcccttgtctatcttcaaaggctaaccatgaattttgaaatcagttgtttctgagttggctaactttgaaagtttctggagctcttcctaccagcccacccccaaacatcaagttaccatttcataatcaacaaatgaatcccaattacatcccattaggaaatttatagtttcaacaaagggcaatcattacctgtaatgggcaaaagatacaatgatcacaaaataggccaagtcaccttttccttcctccccacccacaagagagaagccaaagggtatcagctgtgctttactggctagccacttagcaaaaagtacgtagaagtcctttgtggattacaaaggacagtgtaagcccatttgtgtctttcctctaatttatagttccaggaaaagattataaaaacaaaaaaacaaaaaaatattgtttacattgctcaggggacccgctgagacatggcttgccaggtcccagtccctcccaaatgccacagccattgggagtccacagggtctgcttcgagatctgcacagcagactctgcccagaaagaccacaaatgaaccctcactgagttactacctccagctgccctaagaatgacaataaacctgccttacagaaggaagcagccaccagggtcctgtgccaagcatcaggaaagcatttgggttcttagaggttgccatgaatgctggcccttccagcagggaggcagccaaactggtttggtagcaggccattaccatgggcctgatctcccccaatgagggagtgcaaaatacatgaggattctgactttccaaaccctatcctcaagtaatggtgtcattattacagaaacatagacttgtgatttaaaacgtaatgttttcttctgtcaggaattaagattgaatacatgtcagattaataaattggattcttctgtgcccgtgatttataaaatttcttccataaagcctgagcatccgtggagaagaccaggagagataagggaggagaagagaaagcaagtttctgtccctctgcccttgcctgagctagaacagaatcacatgaaagatggacatttcgttcactgtttcagtttggcagttactcaataagaaatagaattcattgttctggcctcttggggtggtaagagagggagccctactagattggtattgtaattagtttcaagtcccaagacttctgtacacggtcaatagtgacaatgcttcttcacaaaccacataggatcctggaaacggtactcgcagaaagctggctatgatttacttacgacttacactatatgtcaggtgctgtgctatgcattattcatacttgatttcatttaatcctcaaacaattcttacaaggaagatattattaactccattacataaatgggaaaactgaggcccagagagcttagtgacttttccagagtcatacaacagttctcctgggctcctcaatgaggcacttgggctttgcagcaagtagatctgagtttgcatcttgactccacgacttactgcatgaatgaccttcagcaagtctttgccacctctgagtctcagttttcacattagtaaaatgaggagccctttccaaacttaaaaactctttgatgttcaacctgactatatagtcaaagcagttaaacaaaaaggcaaacatgtgagctgtcctgcttataaaagcaatgcagcgttgccaacaagattaagaaaacaatgcatgatcttattttaatatttctatcatcctctgaagccaatatataagaaaagactaggattgggcatagacaagatccttgcctacaggttgcaaaacttggggagagttaccacaaatagcatggtgaatgagttagtgctaatgagtgtggtacagactccgggagacatactggcattcagaaagatacgtggggtcaggaatcattggggaagtttcaagaagaggttgagacattgcagaatatgtaggttgactagctgtggggagaggcaggagctgggtggaagactgaagaaagcagattgcaccctaacatgaggccactctgtttgatttgtgcagacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggtaagcccacctgggtgggatccagccatcctcaagtggtctctctcttgtgcatgtgggtgggccaagcagaaatcctgccccatagtctcctggcttacaagtcagaaaagctcctttgcaccaaagggatggattacatccccatctctttggtcactctgcattgcaaatttcccctcccaccgctatggacgatgtgatgattggaagatgttacaaaacagtggctaaacaaacatgggctttggtgtcagacaaaagtgaagtcctggctttctcacacaccagcttagagcccttggcaaataatgtgatgtacccaagcctcagtttcatcagtaacattgggataataataatatctaccacatcagtttgttgtcaaaattaagtagctcatgcatatactttgagatgcttttcacatgcctgcataaagtaattgttggaccatcgttaatgtctgccataattgcacttaataacaaagcttgtaacctttcaagttctgagattctacaatcttccaaagaaaataaaaggctaatgggaactattcaaaattcatattcagtagcaagcataattaaacatgaaacattaaaaatagaaatttctgtttggctataagaatgcctagacatttgtaatgatcaaaatctgcaggcatcattttctaagagctagactgtaaacaaacctcagaggtaccaactatgccatcagtagtacataaaacatctgatgcacatttagtcacttgatcgatttctcttgaatgagtgaacgaatgaacaaatgaatataagagattaaaattttagccattaagtagaaagaataagaactaaagagaaggtaaaggaggaaaaagagaaggcaaggaagttgagtaagggaagaaatagctctcgtttaagtattttggggactctgttgaaaaaagaaatgccaacatgtggttttaatctttggagctagaactaataatattgtgcaaaagcacaagatgagagatcaagaagttcaccatgacaccttcgctgcttcctggtcttaaacctcagctgaggctggaagaggaccatggtggcttattggagatgtgaccccagggagcccctctgaaggatggaaggggactgggcaagacccaacacacacagaacacagtagccactggccaggcaggaagcaaggatctcagaaaagacttttaggtgaatgtggcaggaaagcgtgcttgctggggcaaaggcagattcattctttctcttcccaggtgacccagcgcctcttggtttctaactggggagggggtaggtgtcaagagatgagtcccaaagttctggaatggtgggtcttgtgactgaggtctagacccctctccagcatgagtgctgtctcctgcatcatatggagcctgggcattctgagctcattcaaagggacaccatgggaaccacttgttctcaatgcaattatttttgtgatgtttacagtatcagcccccatctaccaacaagaacacgaagtctcagagaaggaaaggttggccaaagacacatccaggaggggaacagaaggaggggacagaagcaagtctgcagatcagaggaaagaagaaagagcagaggagggagattggaagtagaaatgctgaatgcagaggcaaaaaaggaaaatgaaggacaggaggattaaacagacagaggcaaggatgatgagagaggagcagacagcaagaatgaaaagcagaaaatacaatagaggaaatgaagaaaagtaggcctgctggagctagatgatgatgtgatggaaatagaagtaaccttttagagaatctcgctaagaaacatggagaaaacggaaaagaaaaatgtaatgccctagaaagcgcaaagaaagacagtggcaaaaatgaaaaaaaaaaataaaaattataaaagaggcaaaaaaagacacactattctctgcctctaaaacacaattaaataaaagaatttaaataaaaattaaggcttctatatgcatttttaaattttgtatgaatctgttatggaagaattgcctatgtcaatatatgttcagagttaaatattagccccaaatgctcagcaagactgaattgtgtcatagaagttcccagattcccttttcccgcaatgtcattggaggctgcatttcttagtcaagtccagggtttaggccaaagggcatccggtattgcctaaaaccctgtgaggtctgtgaggtaacttttgagaagaggtcactgcactcttcatcttttttgcactttggaatcagatataaaagatgtataagtttgctagggctgccataacaaagtatcataggctaggtagtttaaaccacagaaattgattttttcatagttctgggagttgaaagtccaaaatcaaagtatcagcccttgcaagggccttagagaaggctctgtcatgggctcctcccctcggcttgtaggtggcctccttcttctccccctgtgtcttcacttcatcttccctccatacatatctctgtgtctaaacatcctctgtgtgaaacaacaccagccaggttggatttgggcccaccccactgacctcattttaacttaattatctctgtaaagactctgtctccaaatacagtcatattttgacgtactgggagttagggcttcaacacatgaatttggacacaattcagccagtgacagaagacttctgatctctgatgataaccactgcattttgattacagctcctagaaaacactcccctccaccaccccaccacagatctatttttatatctgaaaccctgagtttctgctccatgagaaccccaggaacatactatgttagatctggaagaagcctcagaaatccccttattttgaagactaggacactgagatccagaagtgggtaaagatgtgcttgggttctaagctgctcttcttttggccaggagacaacagcacataatcaaagtgggtcaactaagaaagaattccagaaggaaaagagagggcagaaatgaagggagagaatgagagcaaaagtgctggatttccctgagggtgaagaaaagttaaatagaatcacagaattcagattttagagatcttctccttcagatcccttggtttaatcagtaggattggggtcttcatagataataaagcaaaaactctcgccatcctccaagttgtgaattagaagagctgagaaagggtacaagacggaagttctctaccaaacaaatggtgacattttggggtaagaatatgactaacccagaagtgaagcatttcatccaagtagtctattttgaagatgtcatggtataaaggaacctcctttctgcctggtcctccatgcctctgccatgctttttactccaggatcaccctttctagtggttcactgaaaacccaggattacttaaatatgatggacatgttcacggctcaatccaggaggaaaaggtcgaactgaaagcatgccaaagccccacatgggagccaagccactgctgctgtggttgcaaagtggatcctggcttatcagagcagagagaagccaggctcgtgccttagcccaagtggccagtcaccttattcaggagatactaagttctccagctaagacatccatgctttgggaccagctgcagacagaagccaattcctactacaaccatcaccttagagtagcatatagacacagatggctcttcaaaggaccacagttccatggaataactaagaattcatgtcctgtggaaaggtttgaataaactataattatacccaatcataaatttcattcaagaagaactaaagcaaaggcaaagacagagagaagaaggaaggaaggagggagggagggagggaaggaaggaaggaaggaaggaaggaaggaaggaaggaaggaaggaaggaaggaaagggaaggaagaacaaaaagactttctagttaaagaatgcttaactagcaaactatgtactataagacagttcttttcggaatgagttttatcaactctaaagcaattatcttgaatgcctacatgtgattactgaataatatgaaccaagaaaacagaaagaatctatattatctttccatttccttctttccagtatcaatacccaagcctctagtgatacatggcatataatgttggatggatggatggatggatggatggatggatggatggatggatggatgaatggatggttggatggacaaatgagtaacataggctgatgaatagtggtagaaagacacaccataaaaacaagtggcacttctgagatgaaatgattcctattctcctacacaagacagtgaggcaagtacagagtaaaaaaggaaaggcataggagctatgcttatacaagtattgtatgtttggaatttccttcgctggccaaattgaaattgttcaaggacctattgctacaggtggcaactggctaagaatttcatagtgaatattatacacctattactccccttaatgtttctttgaagtaagcagaatattaataatcatttaaaattccagtgtttcaacttcaattgtttcctagggcaaattgataattgtgtgtaaaactaattggaatatgtatggaataatcatcctgaaataaaattggtgaaaagtatttgttattgggcatctacaatgtgcaaacctctgtactaggcatgaacaagagttataagcattggagaggctaaaatatagtccttaaggctgggcacagtggctcatgcctgtaatcctagcactttgggaggccaaggcgggcagattgcctgagctcaggagttcaagaccagcctgggcaacatagcgaaaccccatctctactaaaaatacaaaaaaattacctgggcatggtggcacgcacctgtaatcccagctactcaggaggctgaggcatgagaattccttgaacctgggaggcagaggttgcagcgagccgagatcctgccgctgcatcccagcttgggtgacagagtgagactctgtctcaaaaaaaaattaaataataaataaatagtaaaatacagtcattaagagtacaaaatgtagattcagactacctgggttcaaatcttggctcttacttgcattgtggctttgggcagatcatgtaacttatgtgtgcctcagtttcctcatctgttaaataggggcaacaactgaatctaccttattcagttgttgtgagggtttattgagattgtgtgtgtgtatgtgtgtgagtgtagtgtgtgcatgtgtgtgtctgtgcaaggagtgggaggtgtatattcagagacacatattacagcacttaaaatggtatctagcacttagtaagcattattcaagttttagttaacattattttacttacctctgaaaattggagctatgtgaaaaagaagttggtctcctgaagtagaagccagtcttgtgtcaccaaaaacttcaagcccaagcttgccaacgcttttccatgatgtggtagtagagtttcaagcatgtggtaggataagagaactcaatgacctaagaaccattccaacccagagaacccctggttctatgaataattccaacttaaataggtagcttggctctcccaagtgagagccattgcttctgtttccgggtcatataatgaactttcagaaaaccaccatttttctcaaccagttaaaattaagtgtaatacgtgctttcatttcatggtgcctggggaaaatttaattgtagtatgaactccagttattggtagtcttaagtaaaattgccaaaataaatagaaatgcaggatatttctgggctcacacagcttccgggacactttagtttcttgggctgccaatccagtgcctttcacaagcatttgatcttttttcaaacatctcttgaaaacaaacaaaacctcacacagcttctaatgtgtgcactgttcgaatgtaagggtggaaaaggaggcaaagaaatgagctcccaaagagcaattccccttctctcgcctccatcccttgacgacctccctcccactaaagggaaacattgttttcttaggtaataaattctgcaatttctcaagtccattaacatccactgggcaagatgagatctattctttttatttgcccataggaaaagaatagtgcttttttgcaatattcactagataacacagagttgacttttaatccaagggcaacattgatagtctctagttaaaggggaagccttcaggagcaatgaaaagattaatagttttagatgaagcagaatccaaatccctttttatgagttttgaaatatccagtttgtatgctcacctcaatacttaaagcccagttactgattcctttggcctaagcaagacaggtcaatttttaaagagggagtagctgaggttagcaaaaattctccaggtccacaaaacttccagacctgcaaggtgaaaatcagcttttctgtcatccctaaaggcctaactggaatcagaacttttccctgatgcccacatatttggaggtccttttttaatgggactccttaatgcctttagtgccatcccattttcatccagtgtccaaaagaaatgatttaaaaatataaacgtatgtttaaattccagaagagagaaatggagattgagaacaatagggaaatgatgagagctatgggaaaagaggtttatgagtccatgtctgattcttccagagagcccctaagaaagttcttatcataccaggaactcaattataactttcattgcctattgttagatgagtaacaggagctagaaaacattttggaaattcccatctttatttttttaactaatatgattatagttttaagaaccattggtcaagaagctaactttttaaaaagtggaagtatgatggttagaaataagaatgctaaaggtgcatcaagctgattttaattctaaatgtccttggcagcaatttagaatctgtaataaactacaccaaacagttttgaggggaaggggattagtttctccccttccttcgtgtgtgtgtgtgcgcgtgtgtgtgtgtgcacctttgtgttctagcattgttgcacccattacagagctggggggaactattttccaaaattataggtgagaacagtttcttggattgtctttcagtgaaggtaaattcctctgtaaaaactaaccatcattcagtaaaaactgcaggattcctttgtcttctcaaaagcctgtttctcatcctaaattaaaaattattcaggaaatagagaggacattattggaggggtggaaataagttggttttctttttattgtatcttttgaggatccagggacttctaccatttcccatctaacatacagagaaggattctctaggtccctgtctatagactgcagtaactttcctatagaaccaatttgcaattttagaaatttctaggtctaattattgacccattacaaccaaaggtcaatgcatccagccaatcttccttctatcatcccctgcccttacttctattagggactgggattacaggcaaaacccatcaaatgcctcttctaccactttcccatttcttaaccattagcctctaacttcctctattcagtttctcatatgctttcatgcccattgggtcagataaaggaacattcatttatttgagtaggcatctgttatgatcactccggaaaaaagatgacaatgggttaccttgtcctcctgggcttctctaactgacatggtcaaaatgcccatatgaagataagatgttaagagcaagatttatgaaaagctgagtatgatggcagctcttgtctcataaaataactcgaaagttcccagtgaaagaccaagaaattttacatcaaacccaaaccggccaaatggtccaagcttccaagctgggatccatggctaaagtttctacaaaattctgggtacaatgtataaacattcacttggggctttctgtctagccagcaccaagaggtcaagtaatcaaggaccaactagccctgccatctgtgaaaatatgtgctattttcacggctttagttcacaattatggcaagacaaaagttccaaataattaggagcaagaccatggcaggttgacggttgagtaaggttctcaatcagccgacaattgtagagttggggatgtgcaatgtttatgtcatggtgtaagtatgtggcatgcttgactagcttgtgaggcactggaagactagaaggaatgaaaaatatgaatgaatcaataaatgcatagtataattactgttattttgtcagtattgttttacctaggtcactattgaatgctctgatttgtctctttataaataataatatgttttcttcttcaaaagaacactaggatgaaggtagaggtgcttttggcacaatgccacaattctgatttttttaaaactgtatgcatgcataaaatgttcttgagccattctctgccttggaatagcactggctggcattctgcatgtttacttttatatgctgaaggcccccatcaacctcaaacagaggcaaatcaatttaacttctcatagtgttattttgttcatcctaaaagttcaagagagccttccaaacttccaaaatttctctcaattcagtgaggaggaaaattcagaacacagcatttgaatgttctgcccagatttgtcacacacacaaggaatgagtgaaagagggcaacaccctttcctcctaaccctgtgaactcatcactattgcattgaaatgacaccaaaaggtaaaaaccctaggcctcacatctcccaagaacactgcaataggagttactgcatacaccagtttaagtaactctagcataaattgtatgtcagatgaaacaatggcattttggaggcttaagagaaaaagaataatcaaatccagtttttaggtactaatgtgctgaatctttagcacatagcagcaaaattgctagaatctggtgtttcactttttaaaataccacatttgaacctttcagcaattccaaaatcaactccctctgcgaaagataataagcttaaacattttttaaatttaaaaatgtaacacaaacaaacagctaagcaaacaagctgcccataaaatcaacagtctggggagccctgatcctgaagtattttacaacatccttcatgactattaaaggcaacataaacacctcttgtcagcaagggaaactacccttggcatttttttttctttgttccccaggcttttaaaccattttgatagagattttttacatcacaggcagaaatatttgaaatagagtcaggtggtagtctttaaaagagtaagaaagttgctaagtcaagataatcttggaataaagtcctctgattcctggggattcctagggatgccccagtcactagaaaacagagctgtaagtccactctcccagcactcaacggagctccggaaaccaaggagctagctactgtttccccacattcagccagagaaagggcagcactctagcatgcaaactgctttgacaatagtaacaattaaaaagtaaattaaaaagaatcataatagctgatattgattaggtacttgccctgtggcaagagctatagggaatcacctcatttaatcttcacatgaagcttgcagagtgagtaccacaattatcactattgtatagacaggaaaactcaggctgagtatggctaagtgtcttgccaacgtcttgggctaacaagcggtcaagcagaatccaaacccgagatagatagaccacagtgtgctaatcaagcactgcactctctcctgcatttcttagttgatatttaccatatacaatctgtcacttgtatgagatggcagggggttctgtgctatttgtccttgtagagaataccacaggaagaaagtaagcagccatgcaatatttgctgttgacctgaactccattccatcattcctgcaggaaattcgcatccattaaatgagcatttcctggtttgccactttgctcaaacactttgcttggatctggagaggatatagaagtgaaggaaatatgctacctgctctcaaggaacttatgttttagtggagagacaaacatgcagaatttactctacagaacatcaatgcttgagcaaatgtagacccagagagggctcttacagcacacaagccagaacagactgatggtgctaacaattaggttcaaggtttttctaaacagtagactctcctgcatacaactataccgcatgccaggtaaatgactgagggttattacatccaattataacaccactgtgatgtaggtgctcttaccccacactttcattttacagaagaggaaattgaggacagcacaatgtagtgattatcaaaggtcacacgactactgtgtgggagagctaggatttaaaccagatgcataagatgaggtcctccaagaaacagaagatgagaaggtgttaaatgagcaggggttttattagggggaattaatgtgtgaacagaaataggggaggataggcaaagccatcagattgcaaggcaagcctaaccccaagggaaggagagagagagagtagattggttggaaacatttttggtgggtctatggtctaaggaaagttcagcaaagtcatcatggagtttttgagccaaagttgggcaatacagttgcccaacaaatttctgtgtttctcagaaataggtctgcctcaatgtccccaccatacttggtcactggctcttgggaggggcctgccctgttccaatccactagagccaaagaagagccgttgtactggcagggggtgggggaattcctacaaccacataaaaagtggggtgaggtttccagaaaaaaacgtgatgctgggctaaccaaaactgtgtccagtaagtacatatccctcactctgttaaagaagcagccacataaacaaggagtacacgtttctcaaaatgtgcaccttgttctttggttttgaagtcacatcccaaagtgctgagtagatcgcatgaccctcgctttgcctggctgccagagaggaaaggctgatccaactctcctggaatttgaacttgtgattccctgaagtaaagagatatcaaagttgatactgagacatctaaatcatcctccaccatttcacatgtccccaggccaagccagcaaaattgctatagcacatccctttcaacaggtaaagggctgatatctgagccctctttccaatcatccactgctcttttcttctcattttgccctttttgggagcaggtcaatgctgagttagtactttatgctgtacaataagctgctgatattccatgctggacagaattttcccagtattttttatagagtgccaggcttttcctagacttcatgtcatacaatacttaacttgtttggagtgggtggagatggaaacatagtctattgaaaacatcactgcttcctccctgaagtttaaagagcctatttttatccttttagattctatctctcaggcaaaatctcataaagataagtggggaggaaaaaaagggggttataatacctagggagtttgcttttgctaattgaatactgtgctcctagacttctataaataccattacaaatgggtcccagcttgtggtaatactcaccctcctcattgagtcttctgtcccatggcacagcctttccctccaaactagcatctacccccatctggaagcatgggcagctcatgatattatcaactattgctattggaaagtgatttggacttgaaagcactagatattttttacctcttggggaggcagtttagcagagtggttaactggtgagctccagaatcagaaggaataggtccaaattccaaccactattacatctccatcataagaaattaggcaagttgtttatcctaagtttcagattccttaaagataaaacagtcaagacagtagtacttatccctgagagaagtataggaaacaagaaaatatatgcaatttacatacatactacaatccccagcacatgacaaatgttcaagtaatgggaactgttattattttagccctttgtctatcagtttgttcctctgtgacctcaagcacattactaaatgttagcgagcttcagcttgtacgtgggactgacaggaataacaccgcatcacctcatgtggtgattgtaaggattcagtgatattattttgtaaactgtaaagcctttgcaaatgttaagcaagattattattattgccgttgttattagtcctcagtgatcttttttttttttttttttttttttttttttttttttttttggagacagagttttactctgtcgccaaggctggaatgcagtggcacaatctcagctcactgcaacctccgcctcctgggttcaagcaattttcctgcctcagcctcctgagtagctgaaactacaggcacacgccaccacaccaggctaattttttgtatttttagtagagacggggtttcaccatgttggccaggctggtctccagctcctgacctcaagtgatctgcccacctcggcctcccaaagtgctgggattacaggtgtgagccaccacacctggcacagtaatcttaattgaaaagtctgtggatagctttccaaaggaaagcttggagcttggataagaaccaagagataatgggagaaggtgaatggcctcttcagggccttttctagcaccctaaatatgcgtgtctgtccataatgggtaatcatatatatcacaaatcaaaccctccacaaacttatttcctaatgtgtttgttaacctttccttctaaagggtaaacttctttaaccaaccccagtgagctggaggatcaatgttttcttaatagtcttaccttcgttggtgtcaataggaaacagtatttactcactactgttttccttttaaaaatctgtctagttgcatactagaaacagtttcagctggtttgtttgtattggacaagctgctgaagtgaaaagtttttgcttgactgaatgtgagacagtttcataactcttcaagaagtgcaccaaaggtgggtgccagctctgatgacggctgcttctaacatgcctccacttgccgcccattgtcaagggtggctggcgtaattaagttaagacaatgagcaaagcaacagatgcaactgagacctagtccctgagtgcttttgttttgtcactgtcattgtctgcaacaaagaagtcacatgtgacagcctgggaagagagccaaatgcaaaccagacgatatcccagctggtttgaatggcctccaccgtgcacgtgtgtgcatgggaatcatgctacttggtacagcatctgcttcactcaagtgagtttcagcccatggctttgctgtgatgctgagacagacccagaagaaacagaccagggaatccctccgctcagactttacactttataccttgtgctttgagagaaaagaaaaagaatctctctattggagacaaaaaataggatgtatgtggttggtcaatctaacctcaattctttttgctatagccccccgctaatttaaagagtgaagcatagatggtatcttaatgttttcttgtagaaatttgggattaatttggcttgagaggaagaatggagattaaacgctttatgaggctttcttttaatttgttcccatttcattcctgaatattttcttagtttgggcattgcagatgtttaaagaacttcttattttgagctggtatgcctcttaaacagaaaaacaaaaggtaaaattcaaattagtgtgtttctccgcctgttaattaatttggttagtagttaggcagagagatggcatccttaataatatctattttgcgggtttgatcagctacagaccatcaacagtgttgattgagaattgaacaaaaacatttcaaggagtttgggaacattagggatgctattctgtggccccatgtgtccttctctcatttttctagagaactcctataagaaagcagaacacggccaggcatgatggctcatgcctgtaatcccagcacttcaggaggctgaggcaggcagatcacctgaggtcaggagttcaagaccagcctggccaacatggtgaaaccctatctctattaaaaatacaaaaaattagctgggcatgatggcgcgtgcctgtaatcccagctacttgggaggctgaggcaggagaatcacttgaactgggaggcaaaggttgcagtgagcctagatcacaccactgcactccagcctgggtgacagagtgagactccaactcaaaaaaaagaaagaaagaaagaaagaaagcagaacccaatggaagattaagaacacacatttagcttacgcctgtaataccagcactttgggaggccaaggcgggtggatcacaaggtcagaagttcgagaccaacctggccaatatggtgaaaccccatctctactaaaaagtacaaaaattagccatgcatggtggcaggcgtctgtaatcccagctactacagaggctgaggcaggagaatcacttgaacccgggaggcagaggttgcagtgagctgagaacgcgccactgcactccagcctgggtgacagagcgagactccatctcaaaaaaaaaaaacaacaaaaaaaaacaaaacacaagtttactgggaacttagcagtagatgctttgcaccacaacaaatgtatcttaagtggtcttttgtgatatttgagggaaagtgccagaatttaaaacaaatggcatttcaagttattctatacaaatgcccagtttctttctaccatctttttttcctttttgcagtggtcactgagctattttagtgaatgtttttacacaatgatgccatcttccttctactcagtcagtacaagatgttgaccatcgactcataaaacactagctacctttcatgaaggacttggtgataactctcatgttccaagtagaaccggaaaacatgtgtaagaaaacctgccgatccctatgggccttggccaataggtattattcccaaggggtggcagtttatctttttccccagccttcatattaaaacctctcaccttctccaggtctcaggtctgtgtaatctcaaatgtgctttagctcctcacaatattgtaactgtgtgggtgttcattaccttagccagaagacagtttacagattccaggtctcatggagagaacttttgtttttggttatgaacctcactgtataccaataattatccattacatccttctgtagagggctctctggctagagataaaaccaaaaaaagaagtacctcaggtttatgcatataaatgccagttcctccttgattttatttcaaaactcctgtctacatactttgcaatttaaatacattcaaggataaagtaataactgtaggaaaagtattataatataatgacttagttctgcacatcacaagggggtccctcatactcattcattcatttcactcattttacagatatttattgagcacctgcaataacctgcacactgctctagacactgggactataacagtaaacagacagatacatctctggtctcacagggcttctattctaagcaaaactcaatatccaggccgggtgcagtggctcatgcctggaatgccagcactttgggagaccaaggccaggcagatcacctgagcccactagttgaagaccagcctgggcaatatagcaaaaccccgtctctacaaaaaaaaaaaaaaaaaaaaaaaaaaaattgtcaaggcatggtggcatgcgcctgtggtcccagctacttaggaggctgaggcaggaggattgtgtaagcctgggaggcagaggttgcagtgacctgagatggcaccaccacactccagcctgggcaacagagtgagaccctgtccaaaaaaaaaaaaccctcactatccttaagataacatcattgcttgttgatgagtgaatgttaacaccaaattaggaacccaggacttttagtcttggcatggttactttccaataaagatgacaatactaagaagagaaaaatgatttaataatgataatagtggctaatacttatgtagtgcttaccatgtgccaggtctattgtaagtacttttatatatattaattatttaatctttgatcctataaggtagatattattgttaccctagtttatagatgaagaaacggaaacacaagagattgccactcatacaagtttacacagccagaaaatagaaaagctacgagttgagctcagcccagtatgtctatgattttacagactcaaaattaattataagatttcctaatcttcgatttctgaaactctgccttgctctagaggaaaacaagaaaaacaatgaaaaataaatgtctcttttttacaaaaattaaaacagaacaaactgcaataaaacaacagaggatgaatccagaatgtgattgattttttttcttactaggaaaggatctagaggccagaaggctggatttttcaggatctcctttcaatcaatgaatctgtgatagaagcagatgaatcaaatctcatctttgtgtgattataaagctgtctgtggtattcacgccaccaggggtacatagaagatgcctgagtgaggtttggcaaaagtactaagggcctgtccacctatacatgcccttctcaggaaaaccaaggttcaagctctctattagctcaactggtaaggcgtaagacatggaaggttgaggcccaatgttagaaatagatggatacataaaacttcatcaagttaatgtcactttttctcctttatttataggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaagaaaaaaaaaatatatatatatatatatcttagtccctgcctctcaagagccacaaatgcatgggtgttgtatagatccagttgcactaaattcctctctgaatcttggctgctggagccattcattcagcaaccttgtctaagtggtttatgaattgtttccttatttgcacttctttctacacaactcgggctgtttgttttacagtgtctgataatcttgttagtctatacccaccacctcccttcataacctttatatttgccgaatttggcctcctcaaaagcagcagcaagtcgtcaagaagcacaccaattctaacccacaagattccatctgtggcatttgtaccaaatataagttggatgcattttattttagacacaaagctttatttttccacatcatgcttacaaaaaagaataatgcaaatagttgcaactttgaggccaatcatttttaggcatatgttttaaacatagaaagtttcttcaactcaaaagagttccttcaaatgatgagttaatgtgcaacctaattagtaactttcctctttttattttttccatatagagcactatgtaaatttagcatatcaattatacaggatatatcaaacagtatgtaaaactctgttttttagtataatggtgctattttgtagtttgttatatgaaagagtctggccaaaacggtaatacgtgaaagcaaaacaataggggaagcctggagccaaagatgacacaaggggaagggtactgaaaacaccatccatttgggaaagaaggcaaagtccccccagttatgccttccaagaggaacttcagacacaaaagtccactgatgcaaattggactggcgagtccagagaggaaactgtggaatggaaaaagcagaaggctaggaattttagcagtcctggtttctttttctcatggaagaaatgaacatctgccagctgtgtcatggactcaccactgtgtgaccttgggcaagtcacttcacctctctgtgcctcagtttcctcatctgcaaaatgggggcaatatgtcatctacctacctcaaaggggtggtataaggtttaaaaagataaagattcagattttttttaccctgggttgctgtaagggtgcaacatcagggcgcttgagttgctgagatgcaaggaattctataaataacccattcatagcatagctagagattggtgaattgaatgctcctgacatctcagttcttgtcagtgaagctatccaaataactggccaactagttgttaaaagctaacagctcaatctcttaaaacacttttcaaaatatgtgggaagcatttgattttcaatttgattttgaattctgcatttggttttatgaatacaaagataagtgaaaagagagaaaggaaaagaaaaaggagaaaaacaaagagatttctaccagtgaaaggggaattaattactctttgttagcactcactgactcttctatgcagttactacatatctagtaaaacctcgtttaatactataaataatattctattcattttgaaaaacacaatgattccttcttttctaggcaatataaggaaagtgatccaaaatttgaaatattaaaataatatctaataaaaagtcacaaagttatcttctttaacaaactttactcttattcttagctgtatatacatttttttaaaagtttgttaaaatatgcttgactagagtttccagttgaaaggcaaaaacttccatcacaacaagaaatttcccatgcctgctcagaagggtagcccctagctctctgtgaatgtgttttatccattcaactgaaaattggtatcaagaaagtccactggttagtgtactagtccatcatagcctagaaaatgatccctatctgcagatcaagattttctcattagaacaatgaattatccagcattcagatctttctagtcaccttagaactttttggttaaaagtacccaggcttgattatttcatgcaaattctatattttacattcttggaaagtctatatgaaaaacaaaaataacatcttcagtttttctcccactgggtcacctcaaggatcagaggccaggaaaaaaaaaaaaaagactccctggatctctgaatatatgcaaaaagaaggccccatttagtggagccagcaatcctgttcagtcaacaagtattttaactctcagtccaacattatttgaattgagcacctcaagcatgcttagcaatgttctaatcactatggacagatgtaaaagaaactatacatcatttttgccctctgcctgttttccagacatacaggttctgtggaataagatactggactcctcttcccaagatggcacttctttttatttcttgtccccagtgtgtaccttttaaaattattccctctcaacaaaactttataggcagtcttctgcagacttaacgtgttttctgtcatagttagatgtgataattctaagagtgtctatgacttatttccttcacttaattctatccacagtcaaaaatcccccaaggaggaaagctgaaagatgcactgccatattatctttcttaactttttccaacacataatcctctccaactggattataaataaattgaaaataactcattataccaattcactattttattttttaatgaattaaaactagaaaacaaattgatgcaaaccctggaagtcagttgattactatatactacagcagaatgactcagatttcatagaaaggagcaaccaaaatgtcacaacccaaaactttacaagctttgcttcagaattagattgctttataattcttgaatgaggcaatttcaagatatttgtaaaagaacagtaaacattggtaagaatgagctttcaactcataggcttatttccaatttaattgaccatactggatacttaggtcaaatttctgttctctcttccccaaataatattaaagtattatttgaactttttaagatgaggcagttcccctgaaaaagttaatgcagctctccatcagaatccactcttctagggatatgaaaatctcttaacacccaccctacatacacagacacacacacacacacacacacacacacacacacacacattcaccctaaggatccaatggaatactgaaaagaaatcacttccttgaaaattttattaaaaaacaaacaaacaaacaaaaagcctgtccacccttgagaatccttcctctccttggaacgtcaatgtttgtgtagatgaaaccatctcatgctctgtggctccagggtttctgttactattttatgcacttgggagaaggcttagaataaaagatgtagcacattttgctttcccatttattgtttggccagctatgccaatgtggtgctattgtttctttaagaaagtacttgactaaaaaaaaaagaaaaaaagaaaaaaaagaaagcatagacatatttttttaaagtataaaaacaacaattctatagatagatggcttaataaaatagcattaggtctatctagccaccaccacctttcaactttttatcactcacaagtagtgtactgttcaccaaattgtgaatttgggggtgcaggggcaggagttggaaattttttaaagttagaaggctccattgttttgttggctctcaaacttagcaaaattagcaatatattatccaatcttctgaacttgatcaagagcatggagaataaacgcgggaaaaaagatcttataggcaaatagaagaatttaaaagataagtaagttccttattgatttttgtgcactctgctctaaaacagatattcagcaagtggagaaaataagaacaaagagaaaaaatacatagatttacctgcaaaaaatagcttctgccaaatcccccttgggtattctttggcatttactggtttatagaagacattctcccttcacccagacatctcaaagagcagtagctctcatgaaaagcaatcactgatctcatttgggaaatgttggaaagtatttccttatgagatgggggttatctactgataaagaaagaatttatgagaaattgttgaaagagatggctaacaatctgtgaagattttttgtttcttgtttttgttttttttttttttttactttatacagtctttatgaatttcttaatgttcaaaatgacttggttcttttcttctttttttatatcagaatgaggaataataagttaaacccacatagactctttaaaactataggctagatagaaatgtatgtttgacttgttgaagctataatcagactatttaaaatgttttgctatttttaatcttaaaagattgtgctaatttattagagcagaacctgtttggctctcctcagaagaaagaatctttccattcaaatcacatggctttccaccaatattttcaaaagataaatctgatttatgcaatggcatcatttattttaaaacagaagaattgtgaaagtttatgcccctcccttgcaaagaccataaagtccagatctggtaggggggcaacaacaaaaggaaaatgttgttgattcttggttttggattttgttttgttttcaatgctagtgtttaatcctgtagtacatatttgcttattgctattttaatattttataagaccttcctgttaggtattagaaagtgatacatagatatcttttttgtgtaatttctatttaaaaaagagagaagactgtcagaagctttaagtgcatatggtacaggataaagatatcaatttaaataaccaattcctatctggaacaatgcttttgttttttaaagaaacctctcacagataagacagaggcccaggggatttttgaagctgtctttattctgcccccatcccaacccagcccttattattttagtatctgcctcagaattttatagagggctgaccaagctgaaactctagaattaaaggaacctcactgaaaacatatatttcacgtgttccctctttttttttttcctttttgtgagatggggtctcgcactgtcccccaggctggagtgcagtggcatgatctcggctcactgcaacctccacctcctgggtttaagcgattctcctgcctcagcctcctgagtagctgggattacaggcacccaccactatgcccggctaattttttggatttttaatagagacggggttttaccatgttggccaggttggtctcaaactcctgaccttgtgatttgcccgcctcagcctcccaaattgctgggattacaggcatgagccaccacaccctgcccatgtgttccctcttaatgtatgattacatggatcttaaacatgatccttctctcctcattcttcaactatctttgatggggtctttcaaggggaaaaaaatccaagcttttttaaagtaaaaaaaaaaaaagagaggacacaaaaccaaatgttactgctcaactgaaatatgagttaagatggagacagagtttctcctaataaccggagctgaattacctttcactttcaaaaacatgaccttccacaatccttagaatctgcctttttttatattactgaggcctaaaagtaaacattactcattttattttgcccaaaatgcactgatgtaaagtaggaaaaataaaaacagagctctaaaatccctttcaagccacccattgaccccactcaccaactcatagcaaagtcacttctgttaatcccttaatctgattttgtttggatatttatcttgtacccgctgctaaacacactgcaggagggactctgaaacctcaagctgtctacttacatcttttatctgtgtctgtgtatcatgaaaatgtctattcaaaatatcaaaacctttcaaatatcacgcagcttatattcagtttacataaaggccccaaataccatgtcagatctttttggtaaaagagttaatgaactatgagaattgggattacatcatgtattttgcctcatgtatttttatcacacttataggccaagtgtgataaataaacttacagacactgaattaatttcccctgctactttgaaaccagaaaataatgactggccattcgttacatctgtcttagttgaaaagcatattttttattaaattaattctgattgtatttgaaattattattcaattcacttatggcagaggaatatcaatcctaatgacttctaaaaatgtaactaattgaatcattatcttacatttactgtttaataagcatattttgaaaatgtatggctagagtgtcataataaaatggtatatctttctttagtaattacattaaaattagtcatgtttgattaattagttcttttggataatgttggtgtaatgtgtcattccttaagaattctactgttatataccatttttattaaggtcattgcaataaataaacatgaaaacatacccagaaaaacaaaactaatcacacacaaaaatgaagacatctcatagaatttacagattctatttaaaagtaaatatgtttcaaaggacttgcaaaagctatgtaagagatgtatgaattgtacaatatatgcattaacaataaagcagttgtacgtaaaatcccctcactgtcacccaacagtgacttataatccctgacacaaagtggttgggaaaatattctattgccagaaggttcacacgtatcaaataaggcacataatatataaatggcatctaatacattcccataccaagagatttataagaatgtgatctctcagcattgagtttttgtttcaataaatcgttccaattttttactgaatagatactgtgaatatcaaattcacaacagcatttatagaagtacaaggtataaagattggtaatctccctcgtttaaaagcatctcatttcattttttaaaaattcattaactatttggaaagt

SEQ ID NO: 10 - DNA, 32809 bp, Homo sapiens
  source 1..32809: mol_type = other DNA
gcatctaggtcaccttcagagccaggtggccattgggttaactagctgttgaactgaatcaaacaggctctgagccttgaagtcagaaagagccagagtggaggggtctggggggaagaggggcgctgaggtatagggagatgaaacctgcggtggaggcagcttaatggtccacctgtcacccaaggaatctctctgacaggggtctttgttagggtcacaccccaggagatggttgattatggctgagtccagcctggaatgatgggggttgggggcagcttgggtagatgactcagtaaatcaaacagaacaatgaaaggaggtcatgcttgtccatctgcattattgaagacagccataaatggccttaccccagagcgggtctgtcacacctggagagctgatctgacctctccaagacccctgcaactgagtgttctgggatctgtcctgcaacaagtgcctcgagatttgtaggtgggggcccagggtgctaagggattattttagcgggcggagaagggagggggtctgcagacgaagggggcaggttttgcggggcacttagggttctcataggttgtagtcacgagctcccaggtttggaaaactgcaatagtcactaagaccattcggggctgtttgggtctagcttggtctagacgggtctgaaactccgcaggacccacccaacaagaagtcattgttccaagccacgtgtcagtggtgggtgataccccaggatggaaggagttggtatgagccgactgaaatctacttgaaggtcaaaacggagccttatgtcttttgtgttccccagcggttagcccagtgccggccacaggggaagcgcgcaaacgaagtcctcgcgaactgaactgagagcagacaaaagcacgcgctcttctccaccgccacgccggtcctacccaaacccgcgagttatccgtattctccttcaggagtcatagtcaggccagaagagtgcggagggacggggcccgggaagagcagggaacccccagagcccgcagccaacgcggaggtgggcgagcgggcgtgcgcgcactcacttgccggcgcgagggagtgtcgggggggaagggagtggtctccaaaagggggaggggagaaggcagggggcggggagaagccggccctttaggacccggctgcggcggcgagggaggaggaagaagcggaggaggcggctcccgcgctcgcagggccgtgccacctgcccgcccgcccgctcgctcgctcgcccgccgcgccgcgctgccgaccgccagcatgctgccgagagtgggctgccccgcgctgccgctgccgccgccgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcaggtaacgcggtctggaacaagtagttgggagaaacttggagggcagcggagaagcccgacgggcggctggaccttacggacggttttaggggcggcagagccgagaccttggaccaaatcaagggggactgttgctagcgggacgcggaagtcaggcccggggaggggaactggagttagagcaagtggaagagccctggcgactcatttggtggggatgaatggggagaggaaaggcatccctgctgccatagttttacccgcgtggaacggacccagacttctattctggaggagggatttgctgagaagggggccggtggtcagatccatgggagtaggaactcgaatcttatttggggcactgcttgaggacccaagggtcctggtgggatcgctgctgggaggcggaggctgcggggtgccagtgacaccaggaaccttggaagaggagagggagctaggaggaggcaccggcgtcccaaggggcccttccttctactggggttgtgagttggaaagaaaggacttggtactttgtaggatgagtcgcttaggagaaaaatgaaaaccaccactactatttctccatagggaacgagcagggctcttgctaccacgttgaatggaaaaataaaagggaggcatttggtaggcgaggggtctcctagcagctgaatcaatgtaacttaagcccgatcgccggctgcattccctggacgggggcagccaggctgtgcggtttcgcatctgggccccagatcgaaaggcaggagagatgcgttgccaccccccacccccgcccctatcttaaattgagggaccctttcctgtaggatagaatgaggacccgatattctttgttcaggggccctcatccctcccaaaaagttatttgagggacacaaccaggctctaaggaggttacttttttttttttttaattacgaaagcctcctgccccagtggtcgggaggaagctgtaattattttctttcctcttggagacagtggtgaaatgtgtccactgcctgctgctgttgttgaaaaagcaagtgagtcttaagtttctcaaacttaaaaagaaaaagtgaaaagaagaatgcttgctttttagcttctgaggtcttcgtaagccccccaccccatgcaccaacccgcctcccttctcctggtcggctattgtttttttccctgttctccttgagaatggaattaggggtcagagttgaaatctttcttctgtttctctagcacattttgctctggagtacagagcaagttagggcttcctctgagtggagcctctcagtccctttcttctccctcggctcgcttccttccctagcctgcctggaagttaacccttccctcctgcttctcttagaaagtaacaataaaaggaaaaagcccccacgaaatcctttcactgcggggtgcctgtcccccagtcatgcgttcctcctcatcacccctcgtggtccaccggggaggtgctgtgcagtttctgagctgaactctgacaaactaaacatcttcctcagcagggtgaactttttgaaaggcctctaagttcataggatggtttggctgctgactgttttaataaagtgtgtgatgcctttccttctttaacgttttacttttttttttttcctactcttcattcgtcttattctaagcctgaaaaccaattctacttcccaaatccctgaagccggttagggtctttagccaggaacagatgcggcagattttctcccattcacttttccctggagcctgaagggggtggggggtagttgtcaggggaggccagggacaatggggtgagttcatgggaagaatgtcactctggattttctgcctggttatgggactcccttccgccagcttgccccagctgcgttctcctccccagtgcccagacgccttccgcatttttgtgcaccggcctgggaagaggggatgttaagcaggggtggaaggtggggggtggggctggaagctgagtgggtgcactcaaaagaagtgaccaatgtcaggggtgcctgagaaagagtcgggctgggggaggggagcactttgggaaggagcctgagcttgcaagggtgcaggagaaaaaggggtcaaggttggaggcttttgtgtgggagagggtcttaggctggggcagagactggagtcactggatggagagtgctgggtccgagttgggtggggcctggcccagacggagatcttagcactgaatcctctgcctcccactccatctcccgtcccgtcttctctcctctccgaagaatgagcaccccccctccagcctgcattcctctttgagcaggttgcttttgagaattttaaacataccctcttatttcgaggcagcgaacctcagcagttccggctttgctgttagctcttttgtctttgttcccgggactcaggggctgtgttgctgctggcttcctggggatggcaaggatgcaggggtgggggtgggcacatcagtgggtggaaggagtgggtggtggtgagcctgtatcccacggggacgatgggctggggcttgttggttcaaagaaactgagaagtcagtcttgggggaaggggtgggtaggcctagaagaaaaaccgaaaggagaccctgagatctgcttgaagttgggactctaaaagcgtgtgtatgtgtttggtggtggagtggatagaagtgcctagaaatgtcagggtcgaggagggaacagggttgggccagtccaggccctttcagaattaaggctgggctagaccggttccttcagtatctccccagcctccttccctaccgtgtacaatgcaggggattatgctgaggggcggggccgcccgagtggccgtttggcccgctgcacgtataaaggtgctacagaaatgtgtagtcgttcaaagtcccagggcgggcagacaggcaggcaggcaggcgggcgggcgggcgggcgggcgggcctggggaggctctgcatctgcagttgccggggaagaataaaggggggaagaggcaagcactgatttacagtggttcagtgcagcttcaaaaaaaaagggggaaaaagcaatgggacttctggctcagagtactgctttgcatctcgtcttgcataaatttgcatactgagttcaaagtcgtaaaatagactagggaaatgagtgggatctactgtcactgattgttaacaccactaccttccacctcccctccccccatctggacagaactcccggcactagttctgctaagcagcgctgcctgagtcaccagtcaccactgttcatgaaagaaggagatggggaagcgcctgtctaggagaccaagcagggtgtggcagtgtgcaccctggcctcggaggcagggcctctctgaagggtgtggcccgacagattctggaccagtgaggcaagtggtgttctttctgacgcctggggctgccgttgcccatggtgggctgcagtttgaggaagctgcagaatccgggtcttctttgtttgaaatttccctactcagcttccacatatgtccaaaacatttactgtgataagtccctttgtgtggtggtaatgacccacttctttagacaagatatttgaggaaggttcgagaacagaaaaattctacttaacaatgttaaggaatctgagaaaagtggggcacaggagtctctaggttagaataggtggtagagcctggaaggagagaagaacccatctgttgtattcccgagggtctgcccacacaatggctcttggccaagtggactgttgagcttcctgtcaggcagagggaagagggaggcttagatagttctcactgttggcctacttcagcactgagcttcaagactttcaggcaaatcattccttcaggctcagaggctggaaattagttctataaaaatgccatggggccggtcgcattggctcatgcctgtaatcccagcactttgggaggccgaggcaggtggatcacaaggtcaggagtttggagaccagcctggccagtatagtgaaacctcttctctgctacaaatacaaaaattagctgggcatggtggagcatgcctgtagtcccagctgcttgggaggctgaggcaggagaattgcttgaacccgggaggtggagcttgcagtgagctgggattgcatcactgctctccagcccgggcaacagagcaaaaaaactccgtctcaaaaaataaataaaataaaaatgccacgggttggccaggcgcggtggctcacgcctgtaattccagcacttagggaggctgaggtgggcagatcacctaaggccaggagttcaagaccagcctggctaacatagtgaaaccccatctctactaaaaatgcaaaaattagttgggcatggtggcacctgcctgtagtcccagctgctcgggactctgaggtaggagaatcgcttgaacctgggaactggaggttgcagtgagccgagatggcgccactccactccagcctgggcaacaagagtgaaactccgtctcaaaaataaaataaaataaaaataaaaataaaaagccatgggttacctgaaagtaggagcatatccattcactcattatctgacctttgagggctgacttggagagaactgttctctggcccagtcatcaggtggcttccagttggttctcagctgggctgttcacacccatagaaaatgggaggtacgactgttcaatttctttttttttttgagacggagactcgctctcttgcccaggctggagtgcagtggtgcgatctcggctcactgcaagctccgtctcctgggttcacgccattctcctgcctcagcctcccgagtagttgggactacaggcacctgccaccacgcccggctaactttttgtattttttagtagagacggggtttcacagtgttagccaggatggtctcgatctcctgacctcatgatccgcccacctcggcctcccaaagtgctggggattacaggcatgagccacggtgcccgaccaggctaatttttttttttttttttttttttgagacggagtcttgctccgtcgcccaggctggagtgcagtcgcccagcctggagtgcagtggcgtgatcttggctcactgcaagctctgcttcctgggttcacgccattgtcctgcctcagcctcccgagtagttgggaattcacgcatgcaccaccatacacggttaatatttttgtatttttagtagaggcagggttttaccatgttggccaggttggtctcaaactcgtgaacctcaagtgatctgcccgccttggcctcccaaagtgctgggattacaggcatgagctaccgcacctggccacaactgttcgatattgatatgaggaagttctgaaaattctgcaccaaagggaatggagaacaggtaaatacctatgatgcctggcaggataggacagggggctgctgaaaggaatgaaggaggagcagaacaaaaaggggctgtgaggccacatccacatctgcttttgttgttggtccttcccgcgtactagaagactgtacccaaaagaaaagaaaaaagcttgttttttttttgtttgtttttttgtttttacaaagagtctgtgtatctgggaattgttatttttatttgtattattatttttattcatttatttatttcaagaccgagttttgctctgtcacccaggctggagtgcagtggcgcaatctcggttcacggcaacctctgcctcccgagttcaagcaattctcctgcctcagccccccaagtagctggaattatagacacctgccaccatgcctggctaatttttgtatttttagtagagacaggttttcaccatgttggccaggctggtctcgaactctggacctcaggtgatcctcccgccttggcctcccaaagtgctgtgagtacaggcgtgaaccactgcacctggcctattttaattttttaaggaactctgtatccagggagtgcatttcgtctcagcctttctctgtatgttcccgactccctcttcctcctcctgccttctccatgctgtcctctttgtccctgcagaccattctttcctttcccacacttctcccttccatgcctttcctactccggctcagctagaacacctgcttgctaggtaggtgggtggggctcaaaaatggtgtggaaaatatagtggtctcagcctcagagatccatccaatcagagggatattatgtaggtgactggagactataatgcttgaaaaaactagaatccttaaatgaggagggtggttgactgggctttgagggaaaggacttctataggccagggaatgggattctctcctataagagttagcactagcgctgcttctgctgaaaccaaggcatagaagcgggagatgatgtttgaattcatgagggaagctggtaacgtggttggcagatattctagatccctctgcttatcctgtctctggccagcaattcactgagatggcaggcagccgccaggagattggtggtgctcgcatctgtcctcaccctattatgaactggcttctgtgcctgctttgtctcctgtcttccacctccctgcgagggaggccgttctacctccattttgcaggtgtggacactgggtgactcagcgatacgaaaatgctgagaggctaacctgggtaccttcccactgcacccctctacctccgtggccgggtaaggagttgggctgttggtagagaaaagcttttgaccgtatcaaccagcagttttgttcctggacagctgccatgtgcaggtgatgtatggggcacagtaaatttctgccagatttcaattatgtagattcccttgactccatggacagttacaagtcctggagtttggggacctcacagttgtaagtagccagcatacagaggtcagtcaagagggacaagaaggtcacagggtgggcctcttgagagaagagctgctgggtagcagatgatggtgcaggcagcctggcctttgctgtcatattgaccggggttggttccattgcaaccctgtctcttaatacttcagtagttagtaaggctcttcctccaatgccagttccctgttggtaaaatagcaatgattttacctctttcccagggtgcgtgttggggtggctgtgataatggcaagtacctggcacatagtatgttcttgtacatgttagctgccttaatcttggcaagtgacatctcctccttgacaattagctggcctttaaaatgaaggccaaactggaaaggtcaggcaggccactcctctctggaggttcaaatcccaggtggtctaccatgtactaatcacccaaggtaagagatttcttgctaagcctcagttttcttatttgcaaatcagaaattgatagtatttcattgggtcaactacacagatgtgtgtaaagatataaactatatataaaacctaagctcttgcattttggtgatgagtcacttgatttccatctcttcacctatgaatggaaatatccatacctctccaacctactttcacagggctgtagtgaggtggtttatggaaagggcaatgaaactgtactttaaatgcaatacaaatgcaattgataaatgcaatataaatgcaactgataagtgcattataaatgcagtgcataaacggttggagaccatcgggcttgcattttttttttttttttttttagacagagtcttgctctgtcgcccaggctggagtgcagtggcgcgatctcagctcactgcaagctccacctcctgggttcacgccattctcctgcctcagcctcccgagtagctgggactacaggcacccgccaccacgcccggctaattttttgtattttttagtagagacggggtttcacagtgttagccaggatggtctcgatctcctgacctcatgatccgcctacctcagcctcccaaagtgctgatgattacaggcgtgagccatggtgcccgaccaggcttacatttttttttttttttcgtgagacggagtctcgctgtgtcgcccaggctggagttcagtggcacgatctcggctcactgcaagctccatctcccgggttcatgccattctcctgcctcagcctcctgagtagctggactataggcacttgccaccacgcccagctaattttttgcatttttagtagagatggggtttcacagtgttagccaggatggtctcgatctcttgacctcatgatccgcccacctcggcctcccaaagtgctgggattacaggtgtgagccaccccgtctggctaggcttgcattttttttttttttttttgagacggagtcttgctctgtcacccaggctggagtgcagtggcgcgatctcggctcactgcaagcccgcctcctggggttgatgccattttcctgcctcagcctccccagcagctgggactacaggcgcacgccgccacgctcggctaattttttgtatttttagtagagacggggtttcaccgtgttagccaggatggtctcgatctcctgaccttgtgatccgcccgcctcggcctcccaaagtgctgggattgcaggcgtgagctaccacgcctggcctaggcttgcatttttaatggattagttgggcagtgctctttggatccgatgtgctgatgtctaggactgactgtttttggtatgggagcttctgtctgcagtgtctagaatgggggttggagggactatggggaacagaggtggccccagagcttggacatctgggttctcatgctggccttgctgctcataagcaggtaacacagggttgagttcctcctttgcctggccagccaacctggaagacctagccttctggctgtcagtgtccagagctctggagaatccctgaggctacatctctgggtgggggaaggtcctctctgtatagatggccctccaggggaagtctgcaaggatccagacttcagaataatagagttaagtattaactaatccaagtggtcctagcacatttagcacatatggcatgctcaagggtgtgcaattgtgcagtcatcaaagccagtgagggcacccgatgccacccatttcctgcaccaggaaaatggacctttcacgtctctgaacctgggatgccagaatgcactgaacttgatcctggcaaataaattggaagctgtttccaaaaaagtgatatgttcttgctacattgtctccagagtttactcctgagttggggagcagcatctcattttgcaaggggctgttggattctccatagaaacgctgggttgggggagtgtcttctgagtactggggctctacctaatgtctgattccatctgtggtaccaaaaagccctctgaatggcagcctgtctgtgcacaatgatgccgaagactttcttttccagcatctaatgaggcatggccccaggaaagcagagaaggcggatgcttgagtctctgagggtcagaatacgcttatggtgaaatcagaactggggacaaggcaaagtactgccattgggaatccggggtggatggtcatgttagaccaggggtgtccaatcttttggcttccctgggccacttggaagaagaattgtcgtgggtcacacataaaatacactaacactaaccatagctgatgagctaaaaaaaaaaaaattgcaaaaaaatctcataatgttttaagaaagtttacgaaattgtgttgggccacattcaaagctctcctgggctgcaggttggacaagcttggcgtagactcttggagggctttcatggtggaggcaggaaaggaaaagccccttttacagatgggatagtaggtccagagaaatgtgttggttttcagttccagagtaatttcacctgctcccaccctccacactcacaaagagcaggctgggtgggatgtcgatttttttttcttctggagaatggaccatctccctatgcctctcctcctccttggctactaacccctgacttttccctcccccttctctggccactcctgttcctgaacaaagccagggaagatctgggctggctcagcccctgggtgtcagtcaagactcagagggagactttgctcaaaaagccagccctcccggaaagaaatacagagcaactgtagtcaggaatttttccctttccttttttctgtgggcggggggagggacacttggcagtctttggggcccttctggctgccagccccacatctcttcctggctgttgggaggcagggcaggagggaagattggggagaagggggcaggggctggcaaacacccagctgctgggcagtggaattcgtccataggccctggtggagttggcctctagtgaagcctggggaggtttctaattagtggaggacggtgaaggtgtggcagcctgtgttcattcaaggaactgagctctctgagccaggaagtcaaagggaagagggggactgagtcccggaaggccttggtaggtggggaggaagttgtacctactcagtgtgttccatcctattagaagttgagaagaggggggtgtttcagctggagtgattcaggttggactctgggggtgcagagagtcagtcattggcactaagcaaagggagccacctcaaagaataagtgatataacagtgataaacaaaatggctttgttctagacattgcctgcttctaccagaaagctcattttgttttttttgtttgttttatattcttcccctctcccacttcctgccccagctctgttttagcctcgagattctctccacttaacaaatgaagaaatcaaggctacattcaggttcaactgccaaggcaagcacagaccagattgtagtctgggtctctagattcttccagctctccatttcaccagactcacacatgcagaaaagcagtgaattatatactgcttcttggaagagctccttttccctgggaagtagtaattaaggcatatcttctcaaaaatgttcctttatgcaactctctaggaagtttcttttaggtctattgtctttacttctggatggaggtgccaagttgcctctgggtggtgaacttgttttctaaaatgcccgaggccaagaacaaaatgaaaatggcaattcccttgtgccctgtgccctgccccgtgacttcctgataatgctgagcagttgctatagcaacagtggtgggaggtgtgtcaggcaggataaagggggtgggggtaggacaggtttactggatcaaagaggcttttgcggttgagtagggctgctgacatgactgatagcctacttctagggaaagatgggggtcccatagagatggggaggggaagaaatgaagccctggggctaaaaggtcttgcttgagagtctttccttaggtgatgccaagtgaccaggtgccatctggttggttaacaacctgggcttggttttggccactggctacatgactttgtgcagttggcctccagctctatagttcatttatcaagtgtgagggggataagaaattaccaacctcatgtgcctgcttgataacaaaggtaaacatgcaagccacgtagcacagaaactcagataatcaaatgaggtagtgcaggttctgattcttgcaatacaggagaacaggagaatggcagacaccgcttttggacgttttctgattggtcatttcaagaatcctgttagcaggtgtgacttattcccttgagtgtggcttacctggacagctgccactttgctttggaaagcctccagtgagtccagagaggagtcaatggttgagagttttccagggggcagtcatgaccctcgatgccattgtgcctgtggttctaccaacagcttaatgaccaaccatgtcccatttatgccccagaagttaggactcccacttgaggctctgctggttgtctctggtttggttcactgcctcttaaaccttgagagcatagccaaggaaggtaattgatgtgatatactatcagacaaaattagtgaactttagttgaagacagagaaaagcatggtgacctatttaaagacacttatttatattcaagacacttcttgtgctggtttgagagtggccggcaatcctggacatctttgcaggttttaaatttctttttgcatcctgtgggttcaatttgtcaggcctggaatatctgagagcagagttatggggatggagaagaggacagtggaaggggagggtggtgggaaaggttggtaggattcagcctttgttacactatgggagtggggctgtgggattgcatcatcgaaactgaattgtttgaatgtgggtcttaggacaggcatgtgtccttcccatcccatctcatggttccacttgctcactgcctttcttgacttgctcactgcctttcttgactcttaaccactcttgctgatacgggctctagatgaggcaaaggccagaagaatgagaaaaagagttaatgtaacagggtactcttgggcaagtgctagatgctctttgatagtgtattagtctgttttcacgctgctgataaagacatacccaagactgggtaatttacaaaagaaagaggtttaatagactcacagttccatgtggctggggaggcctcacaatcatggtgggaggtgaaggcacgtctcacatggtggcagacaagagagcttgtgcagggaaactcccctttataaaaccatcagatctcatgagacttgttcactatcacgagaacagcatgggaaagacctaccccgatgattcaattacctcccactgggtccctcctacaacatgtaggaattgtgggagctacaattcatggtgagatttgggtggggacacagccaaaccatatcagatggtcaccttttctaatgtgtaaacattaattgagcacatgctgtgtgtcagctaggagaccatatgaaaaagcagttgtcataccacctgtaacacatgctttttcatgctggagggggacaagaaaagcattttagaaaggtgaataaagaagaaaccaaggcagcagagatgtatatacatcctatgcaaataaacaggtagctcttggtcactaaaagagcttaaccaagtctcttgggaagagcttgatgttaaataatgaagtcccttcatggtaaataaagttatgaaagtaagagaatcaagttggacaggcttctacttcttcagtcttggaagaaaaaaaaaagcagtgtcttccttcctaaaatgttaattgaggacacagaatgtcttctcacctgtccttcattcactaagtatggaaagctgtacggaagctaagaccaagtttatgtcttcaggaagcttctgtccttaggaggactaagaaaatgcacagacctacagctttttggcaatggactcaggctagagtgctgtgaagaaagttgggtcaaattaccagaagactttacccagcagtgcttttcttttttaaaattttgtaaaatttttatttatttatttattttatttattttgagacagagtccccctgttatgcccaggctggagtgcagtggcgtgatctcggcttaccgcaagctccgcctcccgggtttgcaccattctcctgcctcagcctcctgagtagctgggactacaggcgcccaccaccacgcctggctaattttttgtattttttagtagagacggggtttcaccatgttagccaggatggtctcgatttgctgacctcgtgatctgcccgccttggcctcccaaagtgctgggattacaggggtgagccactgcgcccggcccttatttatttgtttttttgagacaagctctggctctgtcacctaggctggaatgcaattgcacgatctcagctcactgcaacctccacctcccgggctcaagccatcctcccagctatggcagtgcttttcatcaatgcaatcaagtgacctttggacacttggaggccatattgttcatccttcttccttctcaaaaggtttctcctaagttataccagagggagtctgtgcaatttcttcccatctgcaatttatcagatttgttttgttctttcttatgaaattcttggatggcttctaacttggatctctcctgttttagtttaaggctgttttctctctggtccttggggagacagaacggctccttcccttcccttgggttttcctggcccttggacacctccaccactcagcaccctctggcagcaccaggatggatttgtcccttggctttcagttctcaggcctgagtcatcttgattacttcatcttccccttcttaattctgtgaccctcttctactacctttgggtcttctttctgactcatttccgagttcttcatggcatagagaacaggccaggagcccagggctaggttctgactggtggacaagtgaccctggtaacatcattgccctggccagtgagagtggtggtgaagtccgccatgtcactatcgtttggtcctcttccagtagggcttcagggacacttgaggttactgatagctctgcttgatgaggaagatgaggggtggttggtgtgagaggtgacttttttgtttttgtttttatttttgagacagagcctctcactctgtcacccaggctggagtcagtggcgcgatcttggctcactgcaaccttcgcctcccgggttcaagcgattcttctgcctcagcctcctgagtagctgggattacaggcatgcaccaccatgcctggctaatttttgtatttttactagagacggggtttcaccatattggccaggctggtctcgaactcctgacctcgtgatccacctgtcttggcctcccaaagtgctgggattacaggcatgagccaccgcgcccagccctgagaggtgacttttttaaaagaatatttaaaaaatgcagaaaagtacaaagaacaatgtaataacacaagtctaccacccagaaccgtgggcggttgatattttgtccactagcacgcagtacttacttagtgtttatctttcttagctaagcagacttgcatatatgtgatagatgactaatatgcctatgtttgcatatattgcatctatatgcatatatatgatagatgaccaatattgtccaaattgttttgtgaaaataaattgaaaatgcacataaaggttttattttccctcttaacattaggtgtctgagatctaatgttgacatccataggtcagaaatattctttttaactgctatatattgttcctttgtattaccctactggttttatttatccattcacctgttgaaacacttttggatctggttgctaatagtgtttttacaactacaagtaatgctgtaatgaccatttttgtacatgtgtccctgtgcacatgtagagagaagaattgctgggtcaggcacattcttcccattcccatagtccctggttcaagtttgaatcaaagcacagtctactcacttgggcctcagcaactaggtcaccttgccaggttcctggatggctttagttgaacccaaggagaaatgtttgggacccgtggaaaggagtgaaagctgatagtgctgtttgccatcttgatatcagtttctctggctcttcaaggacagcttggctcacggggccccagaggaccttggctgaatttgagcatgtttttgttttctcaaactgtctgcctcagccctgaccctccccaggtctttgcaattccaggaatgaccctgatgtttataaaggagccctttggagagtgcacaggcagggaggctctggagaggctgtgctgtttctgttcactcggggggagcccgagatgagatggcagcatgagggcatactctagaaacctccatgacgccaggccaggagaggcccctctgcatgtatccaagcaagcatatgtgtctgtttacaaacccatgagtggatgagcttacaaggctgtgtctctgcaaagcttatacccgagaccacctctcttgaaaagaagaggcattgttctcgcctcaccaggaagttcttcaggaaacagacagaacagagagaatcacaaaggcatgggggcctggcaatgccagagcttgagaggagaggtatcattcaacatacagattccttagtccttgcctctgactttctggggctggggtccagggctcttatttttagcaagctcatcagatgatttctgatgaagagctatgtttgggaacttctgactgggtctaattccctgaatttacagatgagtacactgacacctgcgcaagttgaacaccctgcttagcgtcactcagaatcccaggggtggtggtgaagtcaggatcgaacctcagtccttcctcccacactgggactgcttggccaaggcttcgaacccaaaagctggtgctccaaaggaaatccaaaaagagtttgcagagtggtttgacgtggaggggattcatcggccaggtgggggctctctgccctcccttggtggtgcatcatctcctgtaggtgttttgtcaccgtggaagggcagtagcctgtgcctgttgggcttgccactcaacagaggtcactgagctcttccgcactcagctgggtgattggcctgtggtagctgcaggtggagggagatgattctcagaagagaaaggccagttcttgcagtcgtgtgatcttataacctaggggacttgtggatctgaaacatctgagaaggctgtttgcagttatttactaccacaaaggaaactgggagaggagggaccacctgttggaaaaccatgctcagaagtgactggtctgttgctgaaggatggaaggatatcaggtgttgcatggctggctcccttgcaggaaggaagggctcagggggtgagaaggaataggaagggagtttacaatgaatgaagctgggtgtcctcaggagatagatggaccagttggggtgatgtcattaactaagctggaatgtgtcctgagtgccagcctataccgcgacagacatctgtgcacactattcagaattccagtgtgagaggccactgccccctctgaccttttgaaaaaaaaagtcaggggaagacacataaacagtgtcactcacccacaaagtctcaggacctcaggttaaactttagctaaagaagtaacaaatagaacccccattttaccttcctctttcctaacttctgcctccctccctcccaccagcatggcctgaattactaatcccagcctgttggtgggctgtgctattgaacatagtggccacatgctagatggagtttggtgaccaccatcagccatccaaagaggactggcttcaaattctctccaacaaaagctgtttcttgtatgtgctggcgtgtttatgagccctgtctcaactgagcatgcgtggtgattatgagctactgcctgacctccttccctttgtccctcccgctaaattctgtaaacaaagttaaagtaatttaggcaaattcattcttgcttaaggacttctattaaatggcagagagctttgatatttagcaatgtataaaatttgtacaatcaaaaccaagctttagaagaaatatgcaatagatcatattgcagaactctcatgactcattttttccatgcatttggccattctacaaatcagacattacctagccttcccctcacatggcagatataacttttatttacccattcaagaaattgattgttcacacattcctttttctttgaaacaaggtcttgctttgtcgcccaggctggagtgtagtggtgtaatcttggctcactgcaagaagtgaactccgcctgctgggttcaagcgattcttgtgcctcagcctctcaagtagctgggacctcaggcatgtgccaccatgcccagctaatttttgtatttttggtagagacaggtttttgctatgttggccaggctggtcttgaactcctggcctcaaatgacccgcccacctcagcctcccaaagtgctgggattacaggcatgagccaccatgcctggccaccacacatttttttctaaagcttggttttggccacagtgagagtttcttgggctgtcaggggtaaaaataccatgtcttgtttaacagcagccatcattttagcagaattttttttttttttttttttgagactgagctttgctcttgttgcccaggctggagcgcaatggcgcgatcttggctcactgcaacctccgcttcccgggttcaagtgattctcctgcctcagcctcctgagtagctgagattacaggcatgcgccaccatgcctggctaatttttatgtatttttagtagagacggtgttactccatgttggtcagactggtctcgaactcctgacctcaggtgatctgcccgcctcggcctcccaaaatgctgggattacaggcgtgagccaccgcgccccgccccttagcagaattgttaatatcatttttcagacttgagaaattagagctcagagaggttaaaggttctgactcgtgagtgaatctcaaagggcaaagctggcattcagggttcttgactgctgggttcattgctcattccagtttgttaatgaatcgtccactgctatggatattaatggagtcgtctagagagcagggggaggaggatggtggttagtatttattctgttgcccaacatatgtcaaggctaagtgccttttgggagaaattaacaaagacggagccatgtgagttttctgcccagctagggtggaaccaggaagctgcggccagccttcattcccctggcccattactcagaattaactcttgaaaggcttggaatgctattacctgttgcaagaggtgagttcattggaggaatgataagttcagggttggcttagatcatctctgtggtactcctctttcccaaccctgctcttagattttaagataatcatgagataggaggagaaggctggaaaatggtaagtctttcttcagaataaagaaaagaagaaagaagagagacacatgagctgttaggaagatgtgattgttctaggtagggaaaaggtcttatttcctcaccaggtctggataaggaccggggccacacactctgaggaggaaggaccagggaggtgcttttagattcttggggcttagtttcctaagaaagctttttaaggtctctcttagatcaggaatcaccaatttccaaatagagcactgaggtcagaacattgtgctaggagaaaatgagattttgtgggagtccgtttctatcaaacacaagagtttgatttgttgagtaagatcattgatgcaacaaacaggattacattggaaaaatgagctgtgccaaaattaggtctgaaaacctgggtatgaatgcatagaagtgaacaataaaggctctttctcttgggctgtcagcttccagctgctctccttggctaaattagtaagggtgggccaaaagacaatgtagggaccccatgataattgtgaagaatggcagagaggttgtttgctcctgtgaattcaaatggtagtccatctgggctcaggtttgccatctgtaaaatgggacaacgatccatttcttttcatcccatctgccttcctcatctccagctctcctctaggggtcttaggtagaaaggtgtggcagtctacccaaggcaggggtgggagttctcccctgcccttcaagcccccgactaatggctctggggttgtctgtcatctccctgacttctctttggaaaagcttgaccttctatatgtgaaatcccttgcttctgtgttgacccatttccctaggtgctatgtagggaagaagagctaatgcttactaatctacagggaaagtctcttcaggggccttggaggtttctatcaaccatttctaagcccatgagccttttccattgatgtcatttggggttgctaagaaccatttcttatagtgacctttgcaccatagcccatggagttgcatacataagtggttgcttaataaatattattctgaacttaactctcttaggcagaaaaaaaatacaacttggtatttttgttctgagggcaaattccatctctagctggagttcgacatctggaatgttttcctgtggattgttcttgcagcctataagaggatatatgggaagaacttggtaacatgtgataatgcccttgtctgactgaggggaggaggggcgctatgtgggtacttcctggtaggcaggaaatggaagtggctaagagctaaccccatgtgtgcctactgtgtgccaggccctctctgtagatgcaggcagcatctctttggatcctcccagttgcccataggaggtgcatactgtttgtccttttacagatgaacaatggaagcacagagagcttaagaaagttgtctggtgctgcacagctcagtagtaaaagcttgaattccaactcaggcagttagattttggggtttgaatctctttggctgggaacagtaacaaggaatgcaggcttttctcagcctacaatgctttctgatctctgcctggtcaactctttcatttcccttccctgggcttctacttcaagatgtggagttcagatctggctacgggatttgagtccagaagcctcttcctgagaaagaaacagatgctgatgcatggtcttgttaaaccatctttaaaagaccttgcatcagcaaaccaggcctgtggatttcttcatcttattggcatgttcgttggcactgctgagatcaataaagaaatggaatgaaacatacccttgaatccaggtggcctctgtgagggtggacaaacttgaccttcacctcagagagtttgggacatctcagggcaggaaatcaactttgtcctcctggaggaacactgccaagttgaccccagcctcctgggacctggagccagggttgcatatgacatctgtgggccctggacactgcctctgtgggcccttctataaaaacatatatttttaaaatttaaaaattattatttttcgtttttgagacaggggctcactctgtcacccaggctggagtgcagtggcatgatcatggctcactgcagcctctaacccctgggctcaagtaatttcccacctcagcctcccaggtagctgggaccacaggtgtgcatcaccatgcctggctatttttttatttttagcagagacaaggtgatatagttaggctttgtgtcgccacccaaatgtcatcttgaattgtaatccccataatccccaggtgttgatggagagatcaggtggaggtaactgaatcatgggggtgggtcccccatgttgttctcgtgatagcgagtgagttctcctgagatctgatggttttataaggagctcttccccgcttcacctggcacttcttcctgccaccttgtgaagaaggtgccttgcttcccggacctttggcttctaccacgattgtgagtttcctgaggcctccccgccatgctgaactgtgagtcaattaaagctctttcctttataaattacccagtctcaggcagttttttatagcagtgtgaaaatggactaatacagtcaggcgtggtggctcatgcctataatcccagcactttgggagaccgaggtgggcctcctgtggcaaggagttcaagaccagcctggccaacatggtgaaatcctgtctgtactaaaaatacaaatattaaccaggcgtggtgacacatgcctgtagttccagctactcgagaagctgaggcaggagaattgctcaaacccaggaggcggaggttgcagtgagccaagatcgtgccactgtactccagcctgggtgacagagcgatactctgtctcaaaaaaacaaaaaaaaaaaatcaaaaaaccatattttataacttgtttaataaatgcaaatgtattaatattatatagtaagactttttaacctaaaagttattttttttttctgatgttaatagaaataaaaatatgtcctaggcactgtgcctccagagcccaatggacaagttggccctgcctggcgccttcaggcccgaatgcacatttatctgtgtgtgcccctctcctttttcaaatgtgtttccagagattgcagagggccttcttctgaggattaatgttggattgacctctgggacgcagccaagggatggttctttggccactgtctgtggtacaagagggcttcgtgaagatggggagctgcaggctgaaagtcctctaggaagcccagggaagatcctgtgccctgcccccaagaaactgtggactattgtggctgcagatggaagggtagcggaggggtcttagccgtggatgcggaatatagtggagctagatttgctttatatggggcctggagccatttcagcaatgggctatcagcttacaggtcttgggctaaaagcctgactccagaagagtgattctcaccctgggcagttttgcctctccggggccatttggcaacgaatgaagacacattttttgttgttccaacctggggagggtgctactggtttctggtgggtagaggccaggaatgctgcccaacatctggagtacactggatgttccaggcattgagttcgggtgagcggctccttcccagaagctcatggatagaggtacacatgagctgcctttcccgtctccaaccagacaacgtgttgttatagccataatagagactgtctgccttcacccaagccacttcccaagagggcagagccaggtgagcctctgagcatcacagacttcatgtccacagattggagagttccagaaatggtggcagggtgcagtaccagcagactcccagagctcctgtcttctctaacaccctgtgagcagaatgcggtgtcgaggtctccagaaacccttcctgcctctgtgggcacaaagctgggtagggtgcggtaagcagggtgatgccaagggtgatctcaggtaccaagggttcgggtgagggcgaactggaggttcccacttcattttgtctttctcattttgatcctgagtagctgggagggagtcaaagggctcttctctatgttccaatgcaggtggtgggagtggggagactttctagctactttgctctcctcctgtgtttttggtgtctgcagagggcaggggcagggagcttgagatggtcccactcaggtctggggacagagaggcctgtctgggaagggcagcggggcggaggcagggaggctggggatgagtgcgtgcgctgcacggcatctgtgtgctgcgttagaggtcagtgttgagatggaaagttgactgaacctgatctcttctgtttgttgtccaggtttgtctgatctcagactttccccggcctttctctttcacccttttgtttctgacctctggggtccatctgaagagggaaggggggctcttctaggagggtgtggacctggtggcgctgaagaaatctgccgcacggtggggtgactcagcgtccctgttcttgggaacctggcaggcagctgagcaggggcactcactcagcctgcctctctgggtccctgggtgtctgctgctctccatgtggaggagcacagagggcagggcaggatggatgaggaaagctgtgggctggtgcctgggtctgattagacatggtggagcaggactccaacctagcctggaggcgcacgctaaatcggccttttcctgccggtggtgctgggctgccgggctgctggaacaatttaatctgtgctggcttcatttatggtacaacccaggctccccagagcccctgtgggaataaccattctaaataaaatatttttccaggtttcaggtttatacatttaacatttaaattttttttttcctggagttcaacaattttggataaaagtcttttaaaatgatgaataggtagaattttaattccctcccctttttgatgtcccaagatcatcgtcatcaacgtaattcttgttttgggctgtaagacatggaaatccccagggttcattgaggggcattcattgtttgccacacacaaaatgtacccggagtttgtatgttttgttttcttagtgtcctctttcctctctgccatttgatccttcttgctactgtccacatgcattccttctttgtcccttcaattttgttgactttcttctgcggtggccttggaaaccagacatccagaagccaggcttcctggagcagcactgttgtgaactgtctcgaggctcctcacgagatgattctcccctcatcttgagtggaaagtgccaacatttgaacatgctcagccgtggtaacagctattaggatgttcagagcttattttccttggtttccctgttctcggttgggttccatgctgctgaagtagtagataactggggcattcgtcattgtcatcatcatcagtgtaataaccagcagcaacatggatatagaatttctgtgctccaggtcgtcttttatttatttatttatttatttatttatttatttatttttgaggcagtgtcttgctctgtcgcccaggctggagtacaatggtgcgatctcagctcactgcaacctccacctcctgggttcaagcaattgtcctgtgtcacccttctgagtagctgggattacaggtgtgtgccaccacacccggctaatttttttgtatttttagcagagatggggtttcaccatgttggccaggctggtcttgaattcctgacctcaagtgatccacatgcctcagcctcccaaagtgctgggattataggcatgagccaccacacccggcctaagtcatcttttaactgcttttctttcgtcatactctcaacagctctatgaagaaagaactgttattatccagcaacttacagttaagaaagtagaggcacagagagggtctgtgtggggagaacacgtgcagctgatatagaaaatggcagagctggactttaacccggctacacccctttaaccagagctgctaccctggggacggcgtgctggcttctaaaggaagctgctgcatggcaagtgaagagtgactagttctcaggcttggagggaggggaaaggacagggacagacccccatcaaatgttccgtatgctggggcctatgctgggcacgccttgacagcaggtgctgctggagtgccagcacccggtgctgtccctggcatggaagaggtgccccctcccttagttggtgagtgagtgaatactcttgtatagagttactctaagaattattcatttgtgttctaagggctgtaagtttttgaaggtgcattaatggggaagaaaaaatgtgcaagattggttgaaaaacacccctctgcacccacccagatcttgttgggacctgcaggagagcagctctgggtgggagggtctggggctgtttgccgtgtctccaaggggttggggctggggctcggcggtgtgaggccctgcccttctggagcttgctggggcttgtctcctggttacagctctgccgtgcgattcagatcctctctgcctcctttggctggacgtgcctcagccagagtgagactgtggcaggctggagctgttttcaaggcctccttcacctaggctgtgcatacaaaaggttgaagaaagtataaggagacttaatggacgcttgttggacagacatcacgaagcttcatgccctgcagtaagctgaagctatgaagtcgaaggagtcatagttcctgttcttggggctctcagtataatggggttggggtgggctgcacagacagacttgggaacgagggaatgtcagtgtggcagagctgtagctgcctcggagcagcactatgggaccttaggcagggccaggtgggcaggtacacagtgacagcagggggatgctgggaccaggttctgggtggcgacgtggccttccaccttctcactcctttgccatatatgctgcagcccggtgatgtctgtggcacgagttttaaaaaactcaactaattggtgctcttccagaccacaaatgaagaactttaaactgggggttatagtgggagaatttgggggggtcccagaaaggacttaaacccattccatggtcagggcctttcaaatgcattcatccctctgtgtgcctctgttctgccccagccttgtaagagggtccctgtcattcttctagagccagacagcctgatttcaaatcctgaatccatcgcttttcagatttgtgatcttggtcaagtaactgacctctgtgagcctcaaattctccatctctgaaagttggggagggtgctagtagtacccacctaagagttagggtgaagctgaaattgatcatccatataaattgctcagcacgggccctgacaggccatcagcactcattagccgcgcgtcatctccaccctcatcatcattacggtccaggtggctcccggagggcacgtctctctgggaaactggacctggagcttttcttcccttcctctcttggcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccagggtcagtgagggtcaggtctggtggaaggggtgggaggacaaggaaagtggggtctcagctggaggagggcatcctaataagaccctcatctggtgtgacctgtaggcaaagcacttttctttccacaaacatagttgtggaacatagttagtgacctttggggcagtcagtatctctggctgtgaaatagactggacatgtggtttcttgatgttctttccttctaaaaccttcacatttctgtatggttttggaacatagacacagaaaatgaatcccagagatgaagacagagtccctgaaacttgcttactcttttttttttcctccccctgagatggggtctcactctgttgcccaggctggagtgtaatggtgccatcacacctcactgctgcctccaactcctgggctcaagtgatcctccttcctcagcctcctgaacagctgggactataggcatgcaccaccacacctggccagttttgtattttttggtggaaatgtgggtctcactatgttgtccaggctggtctcaaactcctggactcaagcaatcctcccacctcggcctcccgaagcgctgggattacaggcgccagccactgcgcctggcctgaaactcagtcttgttgccatatgcttctctcactgagctacccctcccctctttcctgcccagctctgccatcttgggtcctggccctggttggttttatggaaagaagtggagcttgggcatggggtgggggcatggagactcgagacggccacttagatggatatttggaggcagagcccattctgacacatgaaagccacggtcacagaacaaggagaggagtgaacattcccatccatacaattttctttaggggctggggttgggcacccagcatggcagggagatccccgggcaggctgtcagaagtccctgtgccccctgctggctgtgtgaccatgggaaagtcccctccctgtgcagcagcttctcgctgagcttgcgtctggcgcgtgcttcgtgggcctgctgtcctcactccgggcgtcccctgctgtctgtgcgtgcagactccctgccaacaggaactggaccaggtcctggagcggatctccaccatgcgccttccggatgagcggggccctctggagcacctctactccctgcacatccccaactgtgacaagcatggcctgtacaacctcaaacaggtgagcatggtgccagcctgggccagagcagctcctcctcagccctgggccccagatgtgccttgtttctgccccacccgcctatgatcctctgaggtctgagctgagtgagagactcagactcctgtcaccatggggattgggatgccagctgccaggccggggagggtgcagctcttctccctgcctccgacgggtcagttgctggctccacttgcccagctgcccaccggcctcacttggagtccaggttgggaggaggctcttggagaagggaagtggcttaaaccaagcatctttgctttgatcggacattgacatcacctggagctcttcctgggcccgcccccaaattgccatgtggtaggtcaggtttacagctctaacaagcctcaggtgaggctcataatctgtgagccacactcccaccatcattgatttcaaccactggccgagttgtcttcatggtttcgaagtatctcagggaatgaagagtcaggcatggagaaatgccgttggaaagattctgaaaagccatctagaacaccattgtaaatacattggagaaaatgaagtttaagtaaaccactcaggagcccttctccctcctctatccaccttctgttcatatgcattttaacagtcactgtgcatatgcaatattttccctgtaacatactttttttttttttttttttgagacggagtctcgctcttgtctcccaggctggagtgcagtggcgtgatctcagctcactgcaacctccgcctgccgtgttcaagcaattctcctgcctcagcctcctgagtagctgggattacaggtgcctgccaccatgcctggctaatttttgtatttttagtagagatggggtttaccatgttggccaggctggtcttgaactcctaacctcagatgatccccccacctcgacctcccaaagtgctgggattacaggcatgagccaccacacctggctgggcattgggatttttataaagctaccctggtggttctaatggcagtctgggctgacctaaagtgtggatgtgtgaagccaggagatgggaacagggctctgctgatggcttaggggtctcacctctccacccccttcacccaggcaccttgaccctctaaatattagtttagtattaaaagctatttgtgtacatgccctctaaatgtaagctggctaactccatttggagctcagaagaaaaagtctcgatcacttctcatgggaaacaggctgtctgctgcacctttgggtaactaaagtgatttgcaggagtagttagcttatgcataaaacagagaccttggagtgggaatgcctggcttccatctcagctgtgcttcttaccagctgtgtgaccttgggcaagttgcttagcccatctgtgcttcagttttatcatctgtaaaccagggataataatggcatctaattcacaaagtggttgtgaggattaaatgactatatttgttagaaactgaactagcagtgtgtcttgcacacacagtgcctggcacacactgattttgattgcaacatgaggttttacacctgacgctatgactaagcttcagtatgctgggtcccccccactggatgggacctctttgaggctggggactgagtcttttcatcttcgggtgtcctgaatggccttgctaatcagaggggtccctgggcgggcagcatcagcatcacgttggagctcgctagcgatgcagaatttccagctctccagggacctactgcatgggaatctgcattttatcaaggtttcctggcgcatatttgaagttgcgaagctccacccggcagcgcatgggtagctgcaaggggtggctgctcagtggacgccgggttgagggacagaaggaaagttgctggctgcgggctcctccatgctcttctcctctctccccagtgcaagatgtctctgaacgggcagcgtggggagtgctggtgtgtgaaccccaacaccgggaagctgatccagggagcccccaccatccggggggaccccgagtgtcatctcttctacaatgagcagcaggaggctcgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagtaaagtgtgtgtcttttgcctgagtctttggggtcttccagggagagatgcagagcctggcctaggttggcctacccgccatcatccagctttgcccctggctcctaggaaaatgggaaaggccgtttccgtttcccattcatgaaatggcaataagaaggaatgggaacctcctgaatccagatcaaaccctccagtgcagccccaggactcccaggtgaggcttctgcccccatgtggcccctgcctggcgctgtacaggggcaggatgagtcacgggctggggatggggagatgtggagcgtgggtccggcttcctccctcgcacattcagataacactgcagtgggagataaggaacaggaaacgttgggattggggttgtctggcccctgaaataaaggaggctccctcctgcccagggtgagacatttccactcagcccccaaagaaagggagtagggctgtggttaactggcttattcataaggatgacaccactgagaattcagatgctgctctctaaggggcccaagggatttggtgtccagaccagcaaatatcaattgaaacaaagggctatgagtgtgtcaaggtgtgcagg

SEQ ID NO: 11 - DNA, 292815 bp, Homo sapiens
  source 1..292815: mol_type = other DNA
ttgtaaccataagtattcagggttgatactgcttttgaattagacagtgctcatatcagttgcataagaccaacctaaagtagaggatgaaatcttttttctgaacctttttcagaacgtaacttagtgaatatattaaaactaaactttctttgaatgggagtaatttctacggattaatctgtaatctcttagaccacacctaaggtaatgtagaggttgttgtatcataggctttgtgattagagaccactggatttgtctttggaaaagtcactcatgattctttgggctttggtttcctcatcttttataccggcttaataatgaccaccgtagagttgtcatggaagttaaatgaactcatgtaccataagtgaccaatacaatgattgacactcagtgatttatcaataaattaaaacatttattatcaatatgacagagaaggtgccgctaaaatagacaataggtttttggaagaggtgattaaatggatgcaaaatttatggattgtttattccgtctacctttgctgtgtcccctggttgtggcatacacacgtgtgggtataaaatcgtaaatcctatgtagtcgcgtagtgcatgcgcagaaggcttagacacgaaatgtcatttcagcaatgtgcctagagaagctctgacgccgccttggaagtaagtcgttgctgcctgacctttgggcgtctgggacggatgcctatacctgcacccagcagcactggaaggggcccaggcccttcgcagcacagcctatccccagaccgcttagtccttcataacatatatctccacggaaaagggtatttcctcccgtcagaaaaagcgccccagtctggtctgggttggtttttatttcacgttgttgcaagtaggcgaagtcccttctgtctcctcccttggggtaagtggaaaggagtccggcagggggcccgcagtggcctgcacaggggaactgggtagcgagagagttccaggcaattccgggggctgccccacagaagcaggtggggatcgacagtggctctccggcccagggaggagagcgcggtcgcgggtccctcccctcagcctggaggctgcagccgctcgagtcggcccgggtgggggcggggtgggggcggcgcggagggcacggagattacggcggcgccacccgggacatccagggccccgaggccctgggcggtccccacgcgagatcgcaaaccatgacaataggcagtcacccgaggtcaaataaaaacggagtgggtcccccgcgcgccgccgccccccgcgtccctggcggcctcccccgaggcccccggcggcctcacgagcccgcagtagccggtggcgacgtcgcccccgccccacctccctgcgcaagtgcgaggctgccggcagcgcggcgcacgctccggccgttcccggcttccgcgcaaaacttccatcctgtccacgtgaagttgtcgctgccttagagagggggaaagagctgcgggaaaagccggggagtgacgactgcggcggctgggcgcgctctctcattttcttttcttctcctttcccccctgtcgcagtccggagttttggctcctctcctttcctcctccccctcggagccggcttctccctccgccccgcttctcccccgcttgtgtacgctatttgttgtggggtggccgaaggggatgtcctgttttcaccagaggcacagcgcgaaggggaaacttcgacactggaaggaacgagaataaatacttaattacggacgcactgaaccgcggctgggacagacacttcgggaacccgaggcggaccgggcgacgaggtgagtgaccccttcttccaacccccgccccagggctcccgggggagcctgagttgagagaacccccaaactttccgggaaagtgcgcgaggctccgccggggacgccgagcgctgggtactgaggacgcgcagctggacggtgcgtgggcgcctgcgtccccggggggcgcttggaggccgggtgccccacgcctgagggcccgggccgctcggaccgcagcggtgctctctgccctagaagacgtccccaagccccaagggtcccttccgagcctgcctgtcccttccggggtcggcgcggagcctgcgcgtaacggagttcatccagcagtccagcgcgcggcttctacctgcaccccgcctccacctggcagaggcgcgagcatcggggtctcccccacatctttcttatgacgtgtattactttctgatgaccccctagatggtccaggcgcgaggatgctgacccagagtccttcggagggtcacaggcgcctgggctttcccggtgccgggtgcgtgtgtactttaaaggctcgcgttctaatctccaggcactgatcgggcttttcaactgcggcgatcccactttaatagtttttatgtggcgtggactgaatgtctcctgcagtttgccagggtcggtgaaattagaggcgccttgtcagagcagtcgcgttcattggctcgagtagcgggtgccatggaaggcttataacttctccaaaggaagggacctggctgggtagagcaggtttttctctccttccaagcctgctgggtctggggaggcagtggaacttgaaatggctcggatttttagcgtggtgaagcgaggtttggaagtagacgtgtgtgtgcttgttttattctgcgccgcacagcaacccgaactttcgtttggtagcacttgaaagagttttctccctttgtttgcgagattctgaacagctcggagcgattagggaatttgcggaccgagtccggtggcagagctggggcgaaagcagagagcgcaatttaatttttgtcatctcttccctgcttgggaggatagtgtttcccttcaccaccacccctctttctccttcctatgaagacaacggatttgcgcctggggtgagagtgtgtgcgggagagtggtgtggagactgtcctctctcaccgcgtctcctgcgcctctccccgccatcccgagcgggcctagagagtcattcatgaatcttaacctgagggcaggggaggaaggtgcaggtccctctgccctttctgccaaggtgcagaatagcgcccgggcgtgtgttttggttccagagcagttccacgtggagcaacttcgtgtgtgtgtgtgtgtgtgtgtgtgtgtgttgtgtacttgatctgtgaggaggtaacaggactctggtgttcaaacccagtgggccgttggccattagtttgctttcctggctgtcattacagacacttccaaaatctgatacctaagagaaccaacaggttaggtttcacattaagggctcatacttaacagttttctttctcccttaccccttttggcttggcaccctgggatcaacgtaattgttggagcgaaatacacctcctggaatatggcattttgttcctcttctcatctgtggccactttgtgaaccctcgggtgtttgtcagtttcagtgcgggctcctgcgggatttaggtgggagtcttaggagcgtttaacaaccgcgggctccccatcagcagcttctgaagtttcacttacacgtaggtgactgacaggattgaaagttgacgatggtttttttgtttgttgcgttgtttttgtttttaaaaccttagggaagggattgtacttgaattcctttcccgggtacggtttggttttaagcagaatcagtgcctttttttttttttttgtcttttaaaatattattggcaagcttaaacctgaagaaccaaaaactagagggggtggggagagaatccccccaaaaaatatttgatacgtgatacggagcgttttaggagactgcattcaaagacatttgtgtatttttaaaaataacattatcccaagaaacaaaaagcagtagtaacaagagacagattgttttgtgtggagcaagactgccagaatctgatttttatggcaacaatatcgaaagcagacataactacacccacatttattgttataaaccgtaaaaatagtttgttccacctgattaaaagttgtaagtcattcaaagttaaacccgtatttaggaatgactgtacaagaatgttaaaacctttgacagctacagctttgaaagcaacataattagatttgttgaaaatgtctcctttctctgattattagtgatatgtacttgtctttcataataagtaaaggtctacaatcaatatggttttcaaaagcctgttttcctttgtaaatttcctgaaaatagtatgtttcaatatttaaagaccattgtaactaccggttggttcacaattataactaaaataagatttactcagttgttttctttttattagtatttgttgaatatttgaaattgaatttctcatttccaaaataaaacgtctcacatatatgtatgtactcacaattaacacttcttttaagtagtaggcttaagttttaaaatttttaaaatcttaagatttgtataaaaaggagtgattgtataaaaaggaataataattattaattgttaagaaaaatagatgctgaatttcagagggtttctagaagctggagaaaaaaattgcatagaagtttcttttttgacgggtgactggcagatgagttacttgtcattttgctataatttatttttttcttttaataaggctctttgggttaaaaaaaaaaaaggaaaaacaagtactccatttctaagagtttcctttttatttatgttgtactttggggtgtaaataaattttacatagccacgggaaatagagtatccaatttcatgttttcagtctctgttgcaagctctaaatgactgatgtcgtgtctaaaaatatatttatatggcttgatatttcattttaacattttatgcactggaattagattttttaggatttttagaaagatagaatgacagaaagacgatgactattgttataaaaagttagaatgttaggaagttcacattctaactttcaaataatcaaacttattttgccccacaaaaactcacagtttttatacagatttcaagaagaggggtgataatctttttcactacttagttaagtgttaagtaatttaagaatgcaagtatgtattatgctttctcattatcatttttttctgttaggtaaatgcaagaacactggacattctgtaaaaacaggcccctctttataaggagtttattcaccatagttctgtattgcagctgctgatcatatttcatggagctgttaaagcacttaaaacctaaaattaggtactgtctggttgtaaatatttcagatcacttatttaagaaataaatagaaaagtgtgctatcaaaagtaggagacgttttgaatcctctcattgaagagctgaacaaacctctatcaaaacaccttcttcttttttcagtgagaatataatcttgacagtttctttttctaaatggatattattcttacatgtactaaatgctaaactcttataacgtgcctccctgtagagtatcttaaactaattatattcagaaatacagttgggggattttattacaatggttactaggtgaaggaaatcaacaccaggggaatggggtggcattgcagtgtacctgcttctctcatgaatttttcctgctaaactaagaaatgacatgctgtttcaggcatttgccctggagatgggtgagatgcaatatgtgtaatgctgcatcttatagttagatgtgttttaatgaaggggacactgcatagtcattaaatcatttttggagccaaacttggcgtcatttagcttgaacttaagtggaagaaaatgaacaagagttacacattcaaaagaagtacaagcaactttgattgcttttaagaggtttgaagactttgtaaacattactgtcactcaatattgcttgtggagctgtacattaatatatgctttggtgatatgtcattttacgcttgaaatttgtcattcttagtgtttctccatccatctttttattagtaaagagatactgaaaatgaacactactattcttactcccctaacccctttcacacctccagaaaaagagatgaaactgattaatttaaaatagaaaccattttgtgttatcaaaaccacatttatatagtgatttgagacagtttcagagagtgcacctctgagtctcactgtaaccttttttgtcattgaaaggtgctaattgatcttagggtactgacacaatagtatagtttgatatttgaaacctttcagagttggtctggcccttttcttaccctgagatttcagtgcatggatgatgaagaaagacaccattctaaaataccagaaattctcatttttttccaatatgaaatgttttaatacagtatgttcatatttttaaagcttttatttacatacagtaagtaaatttattttaacgtacttttggacagtaggagaaagacctatatgttctatcgtgttagaattttttagtttttttttttctgcacaggtagtttatttaggttataatttttaggcaaagtctgattcctattatcacatgaatattttcaaagtgaaattgcgttaaaccaatgtggaatagcttttgtatcaccaaggcatatattaatgtagatgtcaaatatgagagcatattttcttgagtatatttatatcctaaagtgtatttttaaataaaagtggtcactgtagtctttagataattacaatttggctgtcattattactataatattaatcactatcaccatcataacatcatagctagcatttactcaggaatttcgtgcaaaacactgttttaagcatttatatggattagctaattttaatcttcttaactatgccgtaaattaggtacttttgttattcctattttacagataaggaagctgagacccagtcatgcagcacagtggagccaggatcctaactccccagtatgaggccagcacccttatccttaagcgtgtgctgggctcttgcttccatcagtcatataccaccatttttaggtggtacctgaacattttgttttaataatacttatattttatgtaaggataactagatattagaaaaatttgttaaattttgtattaaacctgtaacttcatgggcaatattgtttgagacaagaccaaacaaagtattgaagtcaaagaaaaaaaattaagtatctgaagaaacgtattaagtaacagtgaacaagaatatggcctaaataaccacagtcatgaaggctggacagcaaatgactgaatttggagaaatgctgtattttgtaatgtttcccaattatcaagaacttatgaccagatcttttaaatatttaactaacatgtggaatcttcctttgctcttcaagccttatccaaattggttaaatgttatcaactttgtattttctttgtttttgttttttgttgttttaatgctggttttgaatctcaaatctgcacatttatgttgaaccaactaataaggcttgaagagttaaagagtgcatgatggacttctggaggcaggtttaaattataatggagctgcccatattttggaaatacattcaatttatctggttatcgcatgtgtaaggttttctgtaggtaaatacttttgctgttaataacctgttttacaattttatagtatttttccactgaagcagtggttttcatttttttatttacttatatactcagccacggatcctttcatttaaatggaaactgatgatatgcccagtagagaaatgtgccactgctctaggtgaagcaggatgtaggtagcctgagaatgactcaccaacagtaccctctcacggtggccgccgcctctggagttctctctaagtgtcttcaatgtatggaaactgctgcaaaaaaattcaagtcttctgacaaaaggggttaattcagagtacctgccctaacatgttcatgtggcatcactaaaaaacagactgtcagatacggtaaaatatctcccagtgtgacatatcagcagaaaggtgtgtctactctcccttctataacttgagtatccgtattaaccagtcttcaaattcgatttagcactgagaaaattaaaactgatcaaaatgttccctgtgtgtagttacaggtctgaatgaggcacaaaggacttgtacctgcaaaggttgactttattaattagaacatctttcctcctttaaagactgtaagaagaaacaccagcagtggcctaacttgacatgactttagattttcacgtaaattattgctactatttctgttatcctttccccctttctttttaaaatgaaagggacatttcttgtgaaagactacaattaaatcataaaaatttacattcatgtgccattaagtttaattctactcacaaaagcaacagtacagagtttgaaattctatccctaatcaagtaggtgtaccacataccgggagggctcattatgcacaaggtcatatatacaattcacagacctctgcatatacccaacggagtgatctattcattacatttcacctctgactttgaactccctaatgttaaaagatttgaaaagaaccgaatgttctgattaagagattgaatatttctaacttaatgttttcagtatgttgaaagtgatgatgacttgggggaatcagcagatctctacattacctaattcttttctcttacatttgaatgcaaatgtatattcatgtgcggttatgactcaagtcattcttgctaaatttaatgacgttgtaggtgaatcacattcagatttccttttgcaggttttccagtaatctaaaacaatgcttctagtaggtaacttaagcatgcaaacctcaataaacctgtcaagaacggcaattctactgttttattttgtttttttttttgtttgtttgtttttgcattaactttagttgataagatgatggtactgttatttttcttagttgactcatgaagaattttaatttaggtctagttttttcccttaattgttgactttagtttttaaaggtttcgttcatgaaaatggttagcaaagttgtgggtacttggtaaatgcttgttaaatgctttttccttatcagtgttgctgaagacttgcaaaattagagtgggatggatagatttcttttctactctgcatggctttgaagactttggagcttttattgtattcttatatttttacataccattcccaggaatatttagagagagaatcattgtaaccaaggtcacaggtctaatcctcaggtattcaaattagctttaggtggacaattgtcctatacacacttgttcgtattaatgttgtcataacaaatatagttataatatcttgatgcctctcctgggggcccattctggactgtgttgaagctgtctctaacactctctcacgtccctaagatattcaaccacatttgttcatgatttttatgaggtccctcctggacttaaaattccttaaaaaatttgcctgctgcctatagggcaaattccaaaattctctgcagggaagataacccttcatactgagaaccttgctcaggttgctcttctcatctctcactccccactcacaacaactctcttttccagctgtatggaaaactgcagttctcaaatacaccttggacatttccacttcatttctttgtgcgtggaatgctttttctctggaaaacatcgcccctctccccctgcaaatatcttttctactgctttttcaatacttaaggtgcaaaatgttagctcctctgataagttcctctgacctccagagtctgagttgaacgtttctttcttgttactcccaaagcatccagcttctacatttaacatagcccttgccacattgaaatgtaatagattacattagtctaaatcctgctttaagctgagaatttactggaaggattagctgtatttgtcatttctgtatcgctaatgcacagtgcactgctggacatatagtcagtaggtagttgttatattcccattgaatgaatgagtagaggtactgggaatgagagcagagattgtgacactgaacatcccctccttgagactgggaacagtgagaagggcagtgactattacaaggtgggactacctcaataacaacctcagggagttttggtataaacccagtagaattgcagctgtgaatccaggcttaggttatatgtatgtatacgtgaatgtagaaatgtgtttatttcttaccactgggaagtcagtgatctggtggataagccaaggatcctgaaaatctctggagatacggtaatttcatagtacttgaaatctgagagactcagttcttatagcacagtgagtaagtagaaagaatatgggggctgggcgcggtggctcacacttgtaatcccagcactttgggaggccgaggcggatggatcacctgaagtcaggagttcgagaccagcctgaccaaaatggcaaaaccccgtctctactaaaaatacaaaaattagctgggcgtggtggtgggcgcctgtaatcccagctactcgggaggctgaggcaggagaatcacttgaacccagaaggcagaggttgcagtgagctgagattgcaccattgcactccagcctggacaacagagtgagactccatctcaaaaataaataaataaataaaaaggaagaagaatatggagccaggcacttgggttcagacctcagctctaacattgatgagtttgtaacatcaaacacattactgaaacagattttgcttcagtttctcatgtgtaacagaacatgcctcatggggttctgatgagaattcaatgagtaaatatatgtaaatatatttagagtagtgcttgatatgagtatcagttgttattactatgattgggtatctttcattagattacccccaaatgttccagagatatttagagctgaaggtcttttttggggggctatggattggtgaccataaatggagtgtccagtagtccaagtataaattagatactgtgggtcatcttggcaaatctgaggttgtatttggagccttaagagaagacactataggtgctcaacagttggcattggccatttacgttgtgcagtaatatttcttttagtaaatatttccgcatacagtaatctagattgttttagagctttaatctgtgtggttccttaactactttaagcaattataagcacgttgtataatggtagtactttatagccaatatttaagtttcctcagttgcttttttttcagccgtaaggtaccaattatgagaatttgtataattttcaatagtcagtgggcctgatgaactcaatgtaaactttttaaaaggtagctttacatattgctatgattcttacctttagagagacagtaggaggtaattagtacctctacttacagaatttatcacttggattcagatataatggctatgtggcaggatggggctgaaggaaaaaggaaagttaaaatattaattgtcaaggtctcatttttacagcttgtctggaactgctaccaggtgtatctgtatagttttaaaaatgataacgttgtacctgaattcttcagtatatttaaataagagtttttcaagctctggttattgacattttgggctggataatttattcaggggatactgtcctgtgcctggtaggatgcttagtggcatcgctggatgccagggaccctccctcccctggtaatgatggtgtctccagacattgtcaggtgatctctgggagcaaaattgcctcttcttgagaacccataacctaagcagatcttaatatcatttgatatagtcaaaaaactcccagggctctgcataagaggattgttctatttccagtcaagtgtggagaatcctaataccttcctcccaagtttaaacatgaagtcaaacaattctcattagtctgtgttgatagattaactttgcacagaggggatttacagatacgcttcacacagattgaccatctccagcatattttccttttctggaaataatatatgagtgggagtaacagaatacctgagagagagtatgtaggaagtaaatatttatttgaatcatctatgtgtcttttcttccggttatttgtcagtagatagtttggatttatttttaaataatgaccattgcctttccatttctgtgggtaaatattcagtaataagaaacttttattttatttagtctgtagtgttagaaaaggtaaagttactgataatcacaactgctgaagattaaaatacttagtgagttaaattatttgtttgtaatgagaaattttcaaaagaattatgtgtagctttcagttgtaaccacaagttcaagatcttgagttaataacatttgtacagctagaagaaagtaaaaataaatatattacatgtaaaaactctcttttgtgaattctcagatccaaacttttaagttctcattctatgggtttgatgttgaccaatacattttatctatgaaatgatatataattgatgtaagtaaatttgactgttgtttgggcaccacccttacataatttaagcatatatgaaacacatttcaaaaatcacttgaacaaaatgggaacaataatgttcacataaagtgagagaggataacttttcccatatgagttttgggttgaatttcaaaattattctgaaatatgaaacattaatattataaatatatgattagactaattttcttaagagttctaaacagtagataatttttatcttattagaaattgcaaagatacttgaaattccacacttatgcattgtctaatttagtggttgtactttaattgtattaggtagataatcatggaagcagtcagactaacctggagatttcacatcatttactaattgatgcctaagtaggcagggtagaggtacttgaaaacacacacacacacacacacacacacacacacacacacacacacacactcactctctctctctcacttcctttgatttgtgctttaattttgaaatgtgtgcttaggtggcaaagggctgcaaatacagcatactgtgtttgaccaaaatatttttctgtttcctaggtggagaaggaccctgcatagctccttgttagcagagagataaagtgggtgctgggcaacccaatgacaggtggaggccgttctcggggagtgttgcggagaatttgagacaggggtttaaatcacagatagtggaggagagttctgcttcattcatggacaggaagtccaaagaaggcacagcgaaccacaggtccatccaggcactttatgccagatattgaacgggaagcctagtagccctttagttactggagaacggccaaaaacccagaggttctatttttccaaatgagcacttcctgggttcataaatcaaggaaccccacagagtattcaagtcacatatctcctttctcaggttgattctttgtatagtgggtcctaatgtggtaagacttacttatgaacattttatttcttataatgccgtcagtcacttccttttttctccctggcatattctcaccacccttatcctccatggccccaactcccttaacctacttgcctagtgctcttttgctgcctacctggacaaaataataataataataataatctactcatctttcaaggcttagctcaaatgtcacctcctctgtgaagccttccttaaggctcaggcagaattagttcttttctctgtgtaataccatagcactttttttggactgttaatataacagcagattgtgttggggctcattgtatgtgtctgtcttctagagagactatgaactcacagaggggagagatcatatctttttccctccgtaacctcaacacctggagatgttaatggaaattaagtgactgcactattccatgcatatctttaaagggcattaggtcccaaaccagacatctgcaaaggatgaatcctgttaactttttggaaaactggttctcttttgcttgtggtcatgaaagctggtttattggttattttctactccaacttatttaaagcctcatttgtaccaaagtattactgattttctagtggaaaaacaggtatgtccggcgtacattggtattttcaataaactgacatttaaaagacagatttctcgttgaggtttatggtcttcataattctagttttctccagaaacagaccttgatctctttggttgccttctgctaagatcgatttcatgttattttgaagaatttttaaccctcagaattatagatttcatttgagagaaagcatgttctgtatttgtttgtgtcagtgtttcaaaatgtgaggaccaaaaatcccagatgtatctattaaaaatatacgtggccttttgctgagggaaattacatcttttttttcaggcacaattgaggaagtaaaaaaattttgtttagtttttagtggtttgtttttaggcattttccatacaaagatgagcaagacccttcaaaaaccaccaatttgcttatttaggggggaaagtcttcctatgtccagaaataacattaaattaagattattgtttccaatagtttcaaaaattgtggttttattttcttgtatgagtcattttttagcatagtgcagtacatgattgcactacatcatgacaaagcatatttgctctgtgtttcagtgggtcactttatttatcagcgtcagatgatcagatcagaagagagatctagcctaacccttccattttgcagatgagatgttgtattatgtgaccttaagaccttaagtcatttactcagaacaaaacagaaaaagagaacttcatgctggggacacttcatgctcccaactctacatcttaccactttatttttatttgtttgtttctgatgaggagttgagaatgctggaatcagactacctttgtgtgtatccaggctctgccactcatcagcgatgtgactttggtcatgtaattttctgagtggctgtaagccatagttccagcatgtgtacatggggaataataacagtactcgcctcatggagttgtaaggaataattgaattcatgcaggaaagacatttggttcagtgcctggcccactataaaagctcaggttatagtttgaattgatatcttggaataaaatgctgagtgtatttgatattcagagaaggaaattcatgctgctaaagcaagtttttttttttaatctgttacttaaaaaatacaaataagtcatactttgtataacatattaaaacagagatgatagaggttacttttgagaggaatgaccagagggagcaagagagaggctttggaatgctgattatgtcttttatcttggtttggttgctggtcatgtgaaagtgtttattcaataaaaattcatcagtgatcaacttttgatttatgtattttattttactgtgcatatgttatactttcttaaaaagttttaaaaatacggatatatttaaagtaaaaagtggatttactcattcttattctctgcaaatcaggataagttgttatacttagatgtgcatcttttcaggcatttttttttctatatatgtgtaaatgtaataactatgtgaggatgtaatttattttcattttacatttttaactccaataggatcattgatttccttaaaacttgcttttgatctgctgaatcttgaatatttttccacatcagtacatatagttttgggtgcttctatttaatgtatgcagatatttcataatttcactgttcccctcttggtgtatgtttacattgcttccatatttttactattactaagtatgttgcactgatgatttttatgtctcttcgtacatctgccaaaaatatctgtagataaaactctagatgcaatattaatagataaatatgtatgtgcattttccatagaaaataccaaaatgctttttaaaaagatttatgaatcttgcatctttaaggtaatattctcatctttctcacaaaaaccaggtgcaaaaatttcaacttaattgaaccttgtaattatttgtgctctggttaagaaaaatggttatagtagtaaattccttgaaactcatatagtaatcggaatactccactatgattaggataaataattctattaacgatatatatttaatatatacatataatttatataattagtttctcacttaagaatatttagtattcatttttatgaatgcatattatatttacttatggttcgtaagcccaaatgttaggaagaaataaatcagtaatattatattcactacatttttttttacttagtaccatcaaatatttaccagaattattaggagaaatggttctaactctcatgtgattggtgagaacttatgaggaatgatatattgttattctagtaagagcctactctgtgccaggcacagttctaagcaccttacatatattacgatagctttttaacatagagatgaaaggtatagacattgtataagtaataaatgacattactccattataaatgtgttgataaatattattatacatgtcacaatatcagttgtgtcatatcaatgactaatttataaatctgttacttattataatctgtttaatgtggaaataaaagttaatgcatcaaaatccagaaaattgctactcaaatgctggctaaagtaatctgatagatacagttttaaaatgaagtatattaatcagttgattttgttgttcgatttttatatgtagaaaattctgtctccagaatattcagttcttgtagtttttggttaagattttgattaatcttcaatggttatctagtgcctttaaaaagtagtccaactcagaatagctaatgtattcctcttgcattgaataaatatggctatcaatttttgtggggttttttttcctgcagaatttcctgttattctaagggattcaataggacttacacatataaaactgaaaattatattactatggggaagctgctttgtgcctttcagaaggaactctcttgctaacttaagtactgtatgtgtgtagaatagctaatattttcctcccaagaaatttcatgtatgcagttaaaacactcttaaattgattaaggatttgttatatatttccagaagcactcaagagtacttttatggttataagagtagagtgcattagaatgccagaaaccaatgaatacgctagaggccttcaaaactgtgcccaacattcctgactttaccatataaacatgtggggtagcctggaaaaacaaaacaactctccttcccccaacgtaggtgaggcctgtgatgataacacttcaactggcttgaatattaatttgatacttttctgttagaagtaatttttattacttagcaaatgaaatggagaggtaataaatatgactgatatttatttcttgagtaaagtattattttacatcagattcgaccagttacctttatcctttttggaaaatgtattatacaaactaccacagtattttgcctattaatcagggaagctaaaaaacgttttataaacgttgaatcaaaactctcactgctgtgagggaattaaatttcaagcaatatactccttttatagggtgagaaactgaagtatggagaaattacaaggttttcccaaagctatacactgagtcatggcagacatagaaatctacaattctgtttgcctgattcaatctgaaacttcatgaaacgtaatttatccatttcctgacatgtgctttagaagtatatttccttatttgcttatttatgctctcattcattcattcatgcatgcatttatgcactattgactatttatgcactcattcactcattcatgcattttaaaagaatcggtgtttttgtatgatacagtcttgatttagggactatgaatattctgaatttatatatcttagatatttcttcaaagaaactcattgcatttttttcacgatcatgaaaaagaaattaagacattaagaaaaccaagctggattgtccaagtgtgggccatagcagtgtctatgggcagcacctgaagctaattcaggagtgaatctagtttgttcaagtgcttgaacaagaacttggctgaagccctggaggagatgcctggggcatagagtggggacgaggaaaaaccatagcactcttagagagtactgtaaggactagtatccacatctctaccacctgatgtctttacatttggttcagtcaagagagagggggcaaattattctgcttctcattctgccatcttcctgatttcagttctcacgctttaaattcttttttattgcgatctttttcttactagtttaaccagtattttcccgcgctttcttcaccaaattcgtttctttccatcagcttgcggagttatattttttgatcgaactgctggggttacaagtaatttgaaatgaggaaagtgtctagcaacttccctcagtccagtaccctgtaacagagacttcagagtacgtagtgatgacacctgccctcctccccttctatagagagtttagtgggcaacttttttggcttgttctcttgttcttttcttgcagccccaagacttaggactcagacttggtttctgcttgtatggctctccatctcttcccttggaagagttctttcccgggggctttttaggtccaagagcatttggacccctgggagatgaacttgtagattctacaaagaacatccaagtaccaggcaaaccacatcttcctgtagcttaaacgttagtcttttttcccccttcaattttactgaagaataatttagtctccaacaaacaagcaaacagaaccctagggtctttctcaaaggtgcattcttgctgctttgaagattatcatcattaatttctatttcagctaattggttttcctttacatttttatttgggtataaatgtgatactttggtatacattaatgttgatctatattaatacgtcaatttataattgagtgatttaaaaataatgatgcactatgacctagtttatatctgccttccacttaatgcatttcctaagcaattatgcttttgtggctgactgaattcccttataattccctgttgggaaacaaagatttttttttaaattgaagaagtggtcttgttaaccaggggcaccgtacttaacttgagagaaaaagattaggaatgacaggaatgacagcatactcctaatatctaacagcctgccatacatgtgaaagcaggacacatagcagggaagcataacgttgagttgactcaaaacaccaaatgtatttgatacattattttctgaattcttacaacttcagcccttttaaaggtctttggatttttagaagctataagggcagtttttggtaaaggtggccctacttctaattcagttacagagtcagtggaatgatttttatattcaaatgctacaggggaatttattgagaggaaaaagggtactcaaactttcctggatgcctcaattaaaaatctggatcaataaaatttcctcaagtgtcccaaatttaaggaaagcaaacagcatcttatttaattacattcttaattagtattcactatagacacaattcaaatcagtaacttggcattagatgaatcagatttattgccatattatataactctcatgtttactttgttgtgtttgggacttctccctccccataataaagaatatgactcacaggtgtcacaccttgattcctgagaaattaatgttatagaaaagttgtttggaaagatataattgtgattttgttattgtttttgttgtcattattttcttttatggggatgggcacacacttcttgagatttaccagatttaccttgtagttcataaattatttctttagatgtatctaatttgctgtttagtttgtttcttgtaaaaaaattcagtgaatatatttttaaaaacatttttatggttctcttccaggtttgtgtgtatatttttttcgtgtgtggcttttcaattttatttctgtttcttttgtgtctgttttaagtatacctatctagttttttccacattgctctgtagtttcacattcttagattcctaatttgttgttgtggctactctctactgtgttttgtttctttatgtagcttataattttttttttctttttgagatggagttgtgctctgttgcccaggctggagtgcagtggcacgatcttggcttactgcaatctctgcctcctgggttcaggcgattctcctgcctcagcctcccgaatagctgggatcacaggtgcccaccaccatgcccagctaacttttgtatttttagtagaaaaggggtttcgccatgttagccaggctggcctcaaactcctgacctcaagtgatctgcctgcctgagcctcccaatgtgctgggattacaggcatgagccaccgcacctcgcctgtagtttataacttttggtggatttctctcttagaaccctttgtactctgtggagcaattttgcattcccctggagtctagggatttcaaacctctaggtcagtctttattttattttatttttttgtcttgaggtttccataccacagatgcagtaaacatttagaatttacatcctgcaaggctgaagttttgatttatctcactcaagaaattattttcttttattctcttcccaaaggctccatcataggacaggcttctttgctgaatctgatggtgggccgagtttttctatcccccctccaataaatgtgacaaactttttagactccttcctctgtggattgcctcttgcttttgcttcctgcccatcgccataaaagaagataatgacaacaaaacgataacaacacaaatgtaaacgtaccttttcctcttaacttttccatatcatcaatttctcatgaattaaggtgtgatacctgaaagtcacatttgccttatcagttatattcatatagttcctttgtcttattgatgtatctctcatattatccctcatatccacttctccaggcctttttccaccccatgtcagtctagattctcttggcttctcacttggagcgtcacagcagcctttaagttgccacttaactccacttcttgttcatcttgtttgtcagaaatctgaagtagatgtgttgttgcaatgatatcacatgtgccattctttgttttgggggttttaaggcttttaaggttatcaaaatattgtaataaccaattgtcttaattgtattttgtacttattctcctgcttttgccaacttaaaatgtcttaacatcctgttaatactgaatgctgatgctttcctattcccttttctttcacatgtccgtctataattgtttattgattatatcagtctctcaacactaaaggcaccagtcacaactttgatgttatacagaatgacaactaaaaggtgacaaatgctaatgacttaatcgaggagttgttcatttttacaagcttacctgagttcagagaattggtaaaagactggtgacttttggctactatctaaatataattggttaatgtagctcctggagatcagtggttagtataatactcttagttgagcagggttaaaaataattacatttgtttaagttgcagatactactctactgtacagcaaagaatgttaaaatgttttaattcagtatatgaagagcaaaatataaaaacaggttttcttccactctcactagcaagcaaagtggtctgtatttttgaaatatattttttgtttaatcataacaaaatataatacaaatgttattaaaagccctattttatggatgaagacagtgatatttagagatattaattagcttttccaagatcatacatttatccagtagtaaaagcaagttttcacaagctgatctattttattccagtctttatttggaatcactaaacttacattccctttagcaaaactatcgtttacatttctagggtttcaagagttcaatgctaaacttgggagatctattcttaaattctctcaaagtgcaacacattttcctcaggatgatcataaagtcatcagatgtgtgttaagcaatatatatagtcatatactcttgatcaaaaaagatttccaggttacacagaagagagatttatggctgagaaaaaggcagattgcttctaagaaaggcttcaaatatattctcttctgacaggctacaggagagccagctagatgaaccattacagtgtgtacaaaatgctcagcagaataatttctcagtactggtagggaagagcaataggagtaacacaagagaaggaaagttggtctgcagagtctgccttagtatccgtaggggtgatgaggttgcttgggctaagaattctttcacatgtctccctgggacaattcactcagaattacttctgtgttttctaggctatctaggcaaccatcaccaattagcaaacacagacaaaatagaacaccttgtttggacctgcttgtcaaaaatcaaatgatcataatttataattccaggaagacttgccatcaaatgaacttatgacaaatgacccaaatgattctcacaaagtagagcttgttgtaacttttagcatgagaagtatgaaccccattatcattttttaaacattttgagttaaatggaccgttttgcaaagtccatgttctatctttatagacagctcaaaaatagctcagcatctagtttgtaggttggctccatttaaggagcttgaagtcaggcaaggtgctcattactattcatccagatctgtttcttggacacctcagctgtttttcttttctacggccagggaggagagtcatgtgcatagtccaagaatgtgcatctccatgtctctgaacagttggccatagaattggagctaggtgttctttgtgccattggctttcctctctggcatgcttcccgggtggttggagccagccagtgcctgatccagcttcacagacccagtaatgaaatttggagggcagagtaggtaactaggtacacaagaattgtaaagcaatgcccatctggacagttgatcacaactataacttctcaataatgtaacaggaatataaactgttagacttcctataccattgcaatataaagaagataattgaaggccgtgaagatagctcatgaaatgcaaggaagaattggacaactaagcaacaggaagagcagagaaacagtgagactttgggagtcacaagatctgggtgcttaaattctgccaggactttcttgccatcttcatttttgctcctctttctcggtcagttctcttctgtctttccctacagaccagcttttttccacatggatatgaaacgtttccaagttttatatcctaggggttttgccactgaaggaggactagcctgatattttcccagactcatatcctaaggaagatgaatcattagatccaagtaatttcgtgcaactggagaagacttgcagaccctacctaatgggttaagctggatatgcatctgtgggagggagatatgttttgtgggtataatacacatgcaagcaaaccttaatattgtacaagatacgcttctgaaaatttacttgcagtttgaaaatctggaagccaaacagtagaccaactgttgtgagtgttagaatttagtgtaatggcactttctgtgacccaggcaactctgcataggcagaaagattcttgaaagccccacccaggttggaaagggtctgtgtcagaggtcatgagtgggactcttcaggcacctgtttccccatcccaaggtctgcacatggactggaacaaaagttttggcagctgcaatttttaaatcttttcccaagggtatctagggatttaaagcagtagggaatgtactcccccactttcccttgttttccttttcactgcagcaagaggcaagtatagccctcagccctgcttgtaccctcacaggagactcatgtacaggtcccgggctctggctgcaggacagtgtgaggctgctgacagggcgagagaatggcgagaacaatggaacaggactgagggagaaggaagaggaatcgtccccagaactactacttgggacggtggtcccagggagcaaaaaacacccctggagccccttcaaggggccttttggcagagcggtactaccgctagacactgccatggctgcaggtaggactgattaaagaatttctgatttcttctgaggacagggtaaagctctgaattgtttttagagactgcattcagatctatatttcggaaccatcactctggttgcactgtgaacggtaacgtccagggagcaggactggagggaaggagaccattacgtggctgttgctcctgcccaagcagggaacaatgatggcggaacgcctgtactggcagagaggagagagaggagggatttggcgaacttttggaggtattggtaggacttggtaatatattgaatgtggagagcaaatgggcagggagtcagtgacaaagcctaggtgtttggctttgatgactggctatgcctgtgccggttgtgtacagaatccagaggtttagcgtttccatgtgcagcgtcggtcctcattccgcggcagcctggtgtggagttgagatgtgccccctccactccatgttcacgtttctttagccagagcaaagcatgcctggacaacctaggtttctttctttctttctttcttttctctagagagagagggtctccgctctgtcacctagaagaagtgcagtgtcgtgatcatagctcactgcagccttgaactcctggcctcaagtgatcagaggattaggtgtacttctctacaaacttctcaaacctccttcagcatctcattccttaagggaccttacagtctctcccacctggacggcgccatggtgcctcattccatttctgagaattgtcttaaaactttatatagaaacttgctattatgtatttaaatgagaagaaatgttgaatttgtaattctaattttctaagacaagagtttgtagctcgcttctagcagagttaaagaaaaagatatgatgaatggtctatatattgaaaactgcagaagaaataatctaatatggaatgggtgaggttgctacaggtggtgcaaaaataattgcggtttttgctgttactttcagtggcaaaaaccgcaattgtttttgcacaaacctaataatactgtttgaaattcatattttctggtggaacagtcttgagatagagcgggaactagattattggctcataaaacagcatgcagttactgttattccttaatcttatatctttattatgaggtaatgcttctcttacttgatttggagaatgttctcatctttttctcatatatttgtgaaaactaaatgttaaaggaattaacataacaaccattgctaaattatgatgtaaaataatactgtgtctgcaattagcctaggtagttttacttggttcaataatataaaatcactaggctcataatattttagtaccttagtgtgattggccaagaaaaaaattcacgttaccataaaaagtcaaaccaagtaacaattaatctaaatttcaaggaaatataaagcaaagtattatgttgtaacatggaacattaatgtaaataactccccaacagttcatgaaatagattctgaactggcagttgacaaagaaaggctagttttggcttaattctcttatgctccaatatctttcctgaagtcttatttaatttcacttgcgttagcaacagtactctgagatggtggtgcatctggaaaattatcacttctttcctgacaatcagtatctttaatttaattcttacatatgctttctggaaatattacattcatactttgatgacgtcttaaaaataatttcaatgtaataattatacatgtgtggtgagtaaattgtgttccgtaaaagatacttttaaatcctaatgtctgtatttgggaatgtgacctcatttggaaacagggtctttgcaggtatgatcaagttaaaatgagattttactggattaggatgggtcctaatacaatgactgttgtacttaagaggaaaatttgtacagagacatacacacacacaaaagaatgccatgtaacgttaggcacggagaacatcatgtgacaatgaaggcagagattggagtgatgcatctatgagccaaggatggccagcaacccccagaagctggggagagacaaaggatgcttccctaggatctccagagcaagcataaatctgccagcaccttgatttaggacttctttcctccagaactgtgagaatagatttcagttctaagccacccagtttgtggtactttgttataggagtcctaggaaactaatgcaacatgctatgttgtaaataaacagtagttctaattggtataaataataattttaagtatattttattattaggtcaggtgagtaggaaattgcctggtagactaattatcaatgttgtattttctgaaaagttaaaatattcccattcagtaaataatacttgtctaaaagtttctatctgtattgtcctaaaacaaagcaaaaattaaaatggagtcagttatgtacctgaaaaatgtgaaaaaggaaagaaaaatcttttaggaaagatttttcctaaaatggctatacatttgagaaagaattttaaaacttaaatcatctggctgtttattccatttttttgtttacattatcagatacattatgtatgatttcatacattatgtatgttacattcaatcaatatttattaaatacctaaaatttgctaggccttgagctaggtattgtagataggtaaataagacaaacattcccttcccctaaagagattattttggcatgaaatttaatctgtagaagaagtgaaagaggccatttgcagagtctaccatgaaaattcattttcactatctgctgtagttttacctgatcctgtgaaaccattttagcatcatttggttttactttgtgcttctgtattatattggagaaaataaaatttacttattgcacagacaacataaaattaattaaagtatgtgaaaccaaaaatgtaattgtttatcaaattttttttagtgtttcccattcatcagtatccaaatacattcttagttgtgcatccatgttagcataactaaaatgtggtgttcttattttttcatctaattcacaatcaacaatgttcacttttccactttttttttttgagacagagtctcactctgtcacacccaggctggagtgcagtggcatgatcttggcacactgcaacctccgcctcctgggttcaagcgattctcctgcctcagcctcctgagtaattgggattacaggcgcccgccaccatgcccggctaattttttgtattttagtagagacagggtttcaccatgttggtcaggctggtctcgaactcctgacctcaggtgatccgcccacctcggcctcccaaagtgctgggattacaggcatgaaccaccacgcctggcccctcatcatttttataattacttttttgatcattaaattatattatacaactgaccatgatttgtttaattaatccactgcaaagatgaggctgtttctagttttcattgtcatgaattattctgtagtttatattttacgagtttttttggggaaaggttacttcttaggaaagttaatactgggtaaaagggcttaaacatatttatagcttttaaagtacagagatggattgctttctaaaaatattagaaaatgacattgttactagcaaatggttgaggacaccgtgttcacagtgtttctgatattaagtattatcaatatataaatagtttgggcattgaatatgtatacaataatccaaaaaacatgtacttattgctcagatttatgtatttcagtaactgaaagtgaacgcttttccatatattagtttattatttacattttcttataataaattatgttctcctactctttcctaatcatttagtgggcatttaaacttcgcctcactaatttttatgtcctcagtaagtgtatatatagtcacatttatttactaaagatattttccataatctgttgtatccttttacttttggggctgtttttctttattcagaaatgcaaaggttttgctttgttttgtttatgtaggcaatgaattgtatggtctctttgcaatccaatgcagtatttttgccatttatgtaatccatgtatctatcagagtaagaaggaacatcagattttcctcggaaagagagcacttggctaatttgttatcaaggtaatgaggagaaaatggctctaaaggagaaggccagccacagttcagtctgggagaatagagggattgacatgaggtgggcaggtgtcaagtcttgggcagctctgtgcctgctgaccagtggccagcacttaactatttgcgtgcatcatttccaaggtgaacctgagccagagtggtcttggttacttgggccaggggacagttcacaggaccaaggatcaatgggtcagctaggaaaggataagcaagaaagggattagcaggaatcaggaccaggctgagatacccagatttgaggggaggaaccagtgcatcaggaggagaagagggagagtgggaggaggaaggtgggcgtgtgagctaagaggcaaaaagctgggcttttccatcacggtttcttagcctccacaccccatgctgtcccaacacaaaaacaccaccgtaaatctagaactctctgcctacttatactcctgaattcaaatgtgtactgctggctgggcatggtggctcatgcgtgtaatcccagcactttgagaggctgaggcaggcagatcacctgaggccaggggttcgagagcagcctggccaacatggtgagaccctgtctctactaaaaatgcaaaaattagccaggcgtggtggcacgctcctgtagtcccagctacttgggaggctgagtcacaacagtcacttgaacccacgaggcagaggtcgcagtgagctgagattatgccactgcactacaacctgggcgacagagcaggacttcatctcaaagacacaaaacaaacaaaaaaaccgaatgtgtactgcatcaactttcagtgtgcttgataatccaccatatcctatataccctcccttcccttttcatatgcagtttaaaattgtgcttttcttcgaaatgatgctttcctagttttggagtattttaaatcaatatctcactgaaactgtcacattatatacatgtgttattattattttctgacttttaagaatgaaaaaaacatacctttgatctgtgtctctgttatatatgcataaaacttttgcaatttacattttaaatttctcttgtaatttttaaaacgtcttgtgatctaataaggaaagtaacatgtactttggaaaatattgcaaagcataaaagaaattgaagaaattaagaccatgcctacttggatgcagtagggaatatatgactggatatatgtaaaactgagcaaaacatctactgatattgaatattcaataaactgaattggcatctgtattagggttctctagagggacagaactaataggatatatatatatatgggagtttattaagtattaacttacacttattattaacttaacttacccttaacaatcacagggtcctccaataggctgtctgcaagctgaggagcaaggagagccagtgcgagtcccaaaactgaagcacttggagcctgatgttcaaggacaggaagtatccagcatgggagaaagatataggctgggaggctaggccagtctctctttttcacatttttctacctgctttatattcactggcagcggattagattgtacccaccagattaagggtggatctgccttcttcagcccactgactcaaatgttaatctgttttggcaacaccctcacagacacacccagggtcaatactttgtatccttcaatccaatcaagttgacattcagtattaaccatcacaagtccaccccttgtcaacttgaacccatatacatctcctgagattatacatcatcttcaaataaagacaataataaggtcataattatacctaacataatacagctgtcctttgtataaccggaaatgcaccaatccccaacccaaatactattacataaagttaacaatatttaaatgccgatatgaagtcaataaatcttatgtcacatgataaaggaaaaggaaataaaggtattttcttagtacaagtgtatacatgcacaaacatgtttttaacaaaagaaggaggaaatactcatgacaattacagtccccgtttctgcagtggtctcgtggtcgtagatgctattgatgactaacttcttctactacccattctgtattccctttgccttcagcaagcacctcaacaggtcatgtttttttttccttgtggagtgacccaaaccttcattcctgggggatctgggccatttctagtcctgcctggattgggctgttgtagtttcccattgaccttaatcatagggcgtggtaatactaagagacaccctaatggatctcctgtattccatgcatactcttccttacctccgttgtggagtaatagactgatttcatcttgaaagtctgggtcagtcagtcaccccagccaacaatgtaactcccctcttagcctgttgacttaaaggtaggaggagcccaaagtgtccaggtggcaatgttaacatccagtttaatggaatcgttgttgtctctcctggtggcagtgttcctccctctggaactaagacctctaggccagcagaaggtaatgtcgtgggaacattttgctagtggaaggaaaaattttgctagtgaatcactaggggtgatggtgagtggtgccacttccacttccacccttgattcgtgaacccgtgaatcctggctatgggagagacagtgccatatattggatgctgattcagagcgtacatggctttctagagaactttgccccagccctgcaaagtattgtcacctagttggcattgtaattgtgacttcaaaaggccattccaccattctgtcaatccagctgcttcaggatgatggggtacatggtgagaccagtgaattctgtgagcatgagcccattgtcgcacttctttatccataaagtgagtgcctcggtcagaggcaatgctgtgtggaataccaagacagtggaaaaggcattccgtgagtccgtggatggtagtcttggcagaagcattgcgtgcagaataggcagacccatatccacagtaagtgtctattccagtgaggacaaacctcttccctttctatgatggaagaggtccaatataatcaacctgccaccaggtagctggctgatcaccccgaggaatggtgccacatcgagggctcagtgttgctctctgctgctggcaaattgggcactcagcagtggccgtagccagtttagccttggaggatggaagtccatgttgctgagcccatgtgtaacctccatccctgccaccatggccactttgttcatgggcccattgggcaatgacaggggtggctggggaaagaggctgagtggtgtccacagaatgggtcatcctatccacttgattattgaaatcctcctctgctgaagtaacccatttgtgagcactcatatggaatacaaataccttcctggtttttgatcactcagagaggtctattcacatatcttttccccagatttctttgtcaccagttttccaatcccgcttcttccaaatctctgaccatccagccaaaaaccattggctacagcccatgaatcagtatataatcacacatctgggcatttctccttcatgcaaagtgcacaacgaggtgcactgctcaaagttctgccaactgggaagatttcccttcagcgctgtccttcagagatgtcccagaaaggggctgtagtgctgcagctgtccactttagaggggtgcctccatatcatgcagaaccatttgtgaatcaggccctagtcttctcttcctctgtcaactgatcatagggaactcccgatgagggcatcgatgcagccttggggagagaaggtagggtggcaggagtagaaaccatggacatttgagccacttcctcatgaacttacttgtgccttcaggacctgctcaaacctgatcacatatatgctactttcatttgatgatagaatgctgctctgtacgaccttctttatggccagatgggtcagaaagctcccagttcatgatagccagttaaggttgcatagtgacttgatgactcgtagtcaaaccttcagtttccaccaaagcccagtaatgggccaagagctgtctctcaaaaggaaggtagttatctgtagaagatggttgggccttgctccaaaatcctagaggcctttgctgtcattcacctatgggggcctggcaaaggctccaaacagcatccctatctgctactggcacctcaagcaccattggatctgctggatcatatggcccaagtggcagaacagcttgcacagcagcctggacctgttgcagagccttctcctgttctggaccccaggcagttttatttggtaaagtgaatgattaaaggtcttggggccaaaatgatctcaacctattttcaaactttaaatgggttagaagcctggctcgctggcatagagctaggcgtggaatatgagtccctagtgggccacttttggtaagcagaactggcactgtgggaggagccaaacgctgggttaaggcacccgatgctgatgctcattagacaccccactcttgttaccaaaatctgtattagggttctctatagggacagaactaataggagatatatatatgtatatatatatatatatataaagggaagtttattaaatattaactcacatgttcacaaggtttcacaataggctgcttgcaagctgaggagcaaggagagccagtccgattccccaaactgaagcacttggagtccagtgtttgagggcaggaggcatctagcacgggagaaagatgtaggctgggaagctaggcccgtctctttttttcacacttttctgcctgctttatattcgctggcagctgattagattgtgcctgccagattaagggtggatctgccttcttcagcccactgactcaaaatgttaatctcctttggcaacaccctcacagacatacccaggatcaatactttgtatccttcaatccaatcaagttgacactcagtattaaccaccacagtgtccgattgtttctttagattgtaatactcttttccatttaaattgtattttcagaaatatttaatgtcagtagatatttcttgaaatgtgacttcaatgattatgttcatctatgatgaacaactttttaaatctaaattgtttttattataatcttttaatttttcttttttagaattagaatttgaacactagaaatgtaccattgcaattttaaattacatttcattgtgaatttgaacacagttttatgataattgaacatatttctgtagggcatgtgcctttctgtttgaattgtaagagctctttatatgttaagaacatcagcccattgtgtttggtatttgcacaggtatttctagatttttctgtgccttttaattttcattgtgtgtgtgtgggtgtgtgtgtgtgcgtgtatgtgttttaaaagagagagcaagagagaggattcagggttacttgggtttttggtgtgtcttctattgctactatgataaaatcctctatctggggatttaaaaatatatgcctatttttcttagagtttttcaactttaattcatttgaaaatattttaatgtatggtatggagtaaagatattttttgaatagttaaccagttatctcaccatagtgtattactaattatctttcccatgtttatttgtaatacattctttaacatattgtctttttaaggttggtcatgtattttatatttttaaaaagtgaagagtaattatttttcagaagaataatcatctgataattcaaattttcctgtaagagaaagttgtcatattaaaatgcacacttactttttcaaattattttatgtacttttgaaagtaaatggttaattcaaactcttgattgttaatctgtcaattgcttgatgacatacctccctcagtaaaatagtgtattaactgtttattgcagactgtgtttagtgcagactatggatatttttgttgatctgtagaattcctgactagctgttcacataccccacttttaccatgacactattttcaagatcattaattgcatcagatatggagacaatattagtggattttcttcttgagagaatgctcctacaagttactaattaccatgctatgtcttgatatgtttttgtaaccttacaaatgaatataaagataacatttttcccctttgtttccatcttcttgttttagatagtcatttttacttgaaggaagctgcttctacttgggagtggcaggagaagtgagaaaaccacatggaagactcccaggatttaaatgaacaatcagtaagtcttcattctcagttttgctgaaaaaagttgcgataacactactgaaaatcatacgtaaaagagatatattgtaagaagtaagtttttcattaaatatatttttaaatgatttttgaatttcctttcaaagaccatgaaacagacctttggagatttatgccgtttctgcttaatgatgatgtcacattttgatgtcttctttgattttctacttggaaaactagtggtggaaagactaaggcattgaaatcatgatgcatgttttagacagtctaaacaattttatatacgtcctttattattttatttcagatatgctggatattgttgaatatcagaagtataaaaggagaaagtatattattagcatgcttgttagtcattttaaggaggaaagaaaccatagttctcaagcgcttttagtattttactagattcattgcaggaccaagaatgcaccagcttgatggcaatccttttaagattctgatttcccaagggaaaggctctttgcaacctttcctcctagcttgttagtggtgtctctgtgacagtagttctttagcgtgtggaactagaggcagtgggagggcttccctcacacccccacgccttcacactactggaagctgtcgtttgagctgttttgcatacggacaactcatgatgttacaagtaacaagacgtgtgcttactgagagtgtgttctgcagtctgatgctgttccttttttttttttttgatgctgttctttattagcttatgtgatcatgacttgatcttacagtcaaatgtttctccatcaggcaactgaggagaagatacttatatctaataatgtggttctagaaaaggtatatgtgatgagtttaattttaagagggatgtatattctggtgccatttgaagttgatttgatttgctttctagtggatattttatttaaaagaaatgatagccatgcctttaatttggatagacatgaccatagtctaatttcaatatatattatattatgatttatttcacatggcatcataggaaaaatgtaacaaaaaattataaagtgaaaaaactatgaaatttgtcaaatggtagagatatattcctctagaattgtgtgacttctttagtaaggtaaacagaaatggctatgggaaggcaagggctttcaaacttttaaaaatgctggttattgactgtaaatattcacagcatggactctagcaaaatggaggttagtcatcatgactgttggaatcagaagtctgtctgtgtgactatggtggacttgaatagttagtagtaaatgttgacaacatcttagtggtgatgcttcattaaaagagtgctgttgatctgaactttgttctgatacaccgagggtcacatacaagtgtgtgtgtatatgcaaatgtttgtatgtatgtgtgcgtatatgttagagtgaccaacgtttttttgttgccagagactgaggattttctagggacatagaactttcagtgctaaagcacacaaagtttgggggaaaaccaggatggttgattctgttaattactatagggtgtgtatatactcaagaaagaattttgttttcaatgcagttgagaatatatttttatgtttaacactaaagtgatgtgttactaaagatgcgtcatatcaccgttcacaaaatgaagcacagaacttgtaaatcagatatggtgtccttctagtgatattttaaggtcatggagctcactatttctttttaaagtatgtttttaaaagtttccagtgatgctagtgagcagtcaggtttgagaacaagaaatctagggagacctgcctaccctctcagcttgaacccatttgctgaatttaaatttcaaacatgtagaccaatgtattcatctcataaatgtgcaaagagatgcttagaactgttaaatgacttgcccatgttggcatagctgataagaggcagagttaggacttcacacggttcttctgactccaagtccaaagctctctccataacatcgacgtgcctcattgcctttgggttatgcatattgtggcatttatagaaatgtcaaaaaaaaaaaaaaaaagaaaagaaaaagaatcagcccctttacatggaaataatttctctttgaatgaaatctttatatgattctaaattttgtggagtatattaatgtctcaaaatattttgaaaaatgatagcatttattaagatatagaacaagtaggggtggaattaaaataaagttcatagttagatgaaagtgaatttactcatatcattgctattttagttcaagagggattctttgggtgtagacatagcagtaggagtcatttgtggcacatgcacactgcatctagcattttccatatacttgctccaattcttaccatgtattccttcattgcaatttttaaggccactattttacagatgaaactgtgacctagagagtgtcaaggtcaaacagctgttaagtagtagagatcagaattaaaccaacttctctctggccccaaaacttttaatggattttgcatgatacaacataataggtagttaagggcatgagctataaatcagacttccctgtcacttaaatgctttaaaacctttgggaaattacttaaactcttcctcctctatggagtggaaggataataaaaagatgtcttatgaagtgcttgcatatgcctcacttagagtaaatattcaataaatgttagctgctatcacaaatacctgagatattcagatgctctttaatgagtattcattcattctagcatatccagctactgaccagacatcccttctgggagttcacaggtccacacactcagcaaatgaagaataaattcaccagctctccttccctctcccagcttttcttcctttgtcctttatgccagtattactcatctcagtgaatggcatcatcagctactgaatgggttatggcagaaaagaggtagtaatcctccttttcttcatcccgcatctcaaagctttgtccactcttaatgtttaatgtcattcaaagctattctttttgctcgagctcccttagtttagttgagatctttattttatctcttgactttcttgttttactttaagttgcttctagagttgtcttcctaaaatacaaccctgatcacatcactaccttcctttttatccttcacaagccctttcttgccttcaagggagaggctcaagtgtgtcatcaagcatacagagcttaatgcttatgtatttcttcagtttttttttttctggtttctgtcagtttctgaatctcccaggttctctgaggtctctatgcatttgcaagtgctttaatatcttcccagaagggtctctttcccaccaccaggataacctgtaattcttgaagactcagtttaggtttccatccctctgagcagccctctcagactacctcctttcttctcacccaatcagccctaccggtcagggccctctgcagtgtgctgccataaccctttggcatttacctctagcccagcacttaccctacaggactgaaaccctatctgtttacttgcctatctcaccactacattataaactacttaagagtaatggtggatctcgttcatctcgtactcttacttcttagcacgattcttgccagtgttcagtaaatgttttcttcaaaagcctttatatacttttagtgaggtatgttaatgagtagcctgacaaattttagaaagccaaaatgttattctcagagtagattactgactcatgaatttatattcgactcacttaaaaaattgcatagaaggtctaacatgaatttaactgcatatgaccaaaataaagttcagtccttcatttttcctctagtatatctggctcctatttggcattctggcttagaggatgtagagaattttaagagataagcaactaaaaagaatatttaactgtcataaatttatctgcagttctattttagagcttcataattctttagaattggtttaagttatagccagttacagcttattacagtttttaatgaaatgttcttgataatagaaatggctgtgaatgtaggttatacctagcaatagctcccttcatactgtgaaatatattgtatttttgtattcaagcagagaatgagcaatatttattatttttatagcttctataatatctttatagattgaggtgttttataatcctattttggtggtccgggatattacttcagattttttctcatccaaaacattcatatcaacatgagatggagtctggcctaaattaatgatcaagccagcaggtttgaaaagaaatatattttattaatgttaagtattgtatgttataaagtaaaacattcgtgaatattaagtaaaactaagtaaaatgaataagttacagcaggattgctttccctaaatgttatgcattacgggcctgaacattcatatgtatttccacattgttactaaatcaattcacttatattgttggtggcatgattttattagtgacatctaaattaactcacattcattggattatgttacatattgtatgattcattaatacttctggaaattcatttctgtgtccatttttaaatataatacatttatatttcaaagctattctatttgtatttatgaaacatattcatagacatgtttcagtaatacttttggaaatttgtttctacatgcatttgtagatatgaattgtatttatttgccttcaccaaccatgctaatttaataatgtctattaaaattctagagctagtttagtaaattaatttatttttatctctctcttgtttttccttagagtactcatgactttgattagtcctcagatctttgttcctagagatcctacagtattatctttgattatagccactagcccatctttaaaacgtgtactcctctctgtatcagatttttaactattgtatagaattgaatttgccattaaacttatcatctagacatagaacttcagaaccaccaatttatgacagtgtttaaacagtatgctagaatataattattttgattaatttttgtttaaatcatttttctatttttataatgtgacattatcaaaatcctaaaattctattattctgttgctttattcttgggtactctaaacccattcgaccagaaggcatacttgtactgaatatcttaaatatattttacttagatctaaacaatgttggattattctttttcaggatccaattctgtatcgaagaacttgacctttaaactagttaaaagaatcttgtttacaacatgtaatttttatactatccaactgaataacatacttcagagttctgtggaattctgttcaaagcccagtttcagaattactgttaactccaaattgccaattacagcttgactttgcagttatgtagtagagtacctaaactgaagttacaaagtagatgcgttcagtataatttaatgtttcataatcattaaggcttactcttcctttttaagcatttttaaaataatagtttttcttttttctttttttttgtttgagatggagtcttgctctgttgagtacggtggtacaatctcggctcactgcaacctccgactcccgggttcaagtgattctcctgcctcagcctcctgagtagctgggactacaggcatgtgccacggcgcccagataattttttgtatttttagtagagatggagtttcaccctgttagccaggatggtctcaatctcctgacctcatgatctgcctcgttggcctcccaaagtgctgggattacaggtgtgagccaccgcgcccggccaatagtagttttattgagatgtcattctcatacaatacagctcacccacttaaaatgtacaattcagtgatttctagggtattcatagagttgtacaattattaccacagtcaattttagaggatttttaatcagttgagaaagagatcccatgcccattagcacactccccattcccccgatgagcacccctcagccctagacaagcaccaatccacttcctatctctatggatttgcctattctggacctttcatataaattgagtcatacaatatgagaacttttgtggcttgctttttatacgtgacataatgtttgcaaggttcatctatgttgtagcatgaatcagtacttagttcctttttatggccaaataatattccattgtatggagacacttctatttatactctttaaatagaaagaatataaatttgcccctatgttataaggttacttctttttttacaattatgttttaagagtcatttctgtattctagataaaagtccttcaccagatctacacttttcaaaggatatctcccattttgtgggttgtcttttcactcttgatagtgtcctttgatgcacagaagttttaaatttgagtccagtttatctatttttttcttttgtcacttgcgcttttagtgttgtgtataagaaatctttgcctaatccagggtcacaaagatttactcctatgatttcatttctctatgatttctcatatgtaagcgagaacatgtgatatttgactttctatatctgagttgcttcagttaagataatggccttcacaaatacttactcctatgatttcttatatgaattttacagttttctatagttttagctcttacatttaggtctttagttcattttgagttaaattttattgaggaagatttttttttaaacttcaacttagtttagatttcaggggtacgtgtgcaggtttttgtcatgggtatattgtgtgacactgaggtttggggtatgattgatgccagcacctaggtagtgagaataatacccaactggtagttgttcagcccttgttcctcttcctccctgccacttccggtagttcccactgtctattgtttctgtctctatgtccatgtttcctcactgcttagctctcacatctaagtgagaacatgtggtatttgttttttttttcctgtgttgattcacttaggatgatggcctccagctgcatccatatggctgcaaaggagatgatttcattttttttatggctgtatagtatcccatggtgtatataaaccacattttctttatccattccattgttgtgggcacctaagttggttccttgtctttgctattgtgaataatgtctttctgatagaatggtttcttttcctttgggtctataaccagtagtaggattgctgggtcaaatggtagttctgtttaaagttctttgagaaatctccaacctgcttcatagtggctgaactaatttacattcccaccagcagtatataagtgtttcctttttttctgcagccttgccaacatctgttatgttttgacttgttaataatagccattctgactggtgtgagatggtatttcattgtggttctgatttgcacttctctgatcattagtgatgttaagcatttgttcatgtttgttggccgcttgtatgtcctcttttgagaaatgtctgttcatatccttttcctacttctaagtattttttaaactctgaatttttatatttttgtgtgagtttatgaagattcagcacaagatttcattaattattatcttctatgaacttctaaaattataatttgatttgttaaaattttttctcaaatcttagaatatatatgacacatatgtgtgtgtatatatatatatgtatatatggagagcctcatattatacagaatatggagtgtgattctatgtcatttgccaagatctggagactaaatattacgttttcttctgtcaactgtgatcagctggtaacagctgtttgtgttaagaaggaacctatggctacatccgagctcattggaaaagtaatgttatcaatttggtgtgatttgccatggacttggtaaggaagaagggtagcaccaagcctcataactgctaattctggtctggtgagttgtcaagatccctgggcagttggtttggatagtatagatataaataagagctatgatatctttgggtaccaaaagtcttgtgtctgaacttacaatttacatattgttatctaataattctatcttataaattaaaatgaaaataattcaggctgggtgcagtggctcactcctgtaatcccagcactttgggaggctgaggcgggtggatcacctgaggtcaggagttcgagaccagcctggccaacatggtgaaaccctgtatctactaaaaatacaaaaattagccgggcatggtgatgcatgcctgtagtcccagctacttagaaggctgaggcatgagaattgcttgaacccgggaagcggaggttgcagtgagccgagattgtgccactgtgctccaccctgagcgacagagcaagattctgtctcaataataataataataatataaaaaataaaatgaaaataatttttatgatagaaatataatctctttcatggtttaatactcttgagaaatagaaaacattaataaatataatgcaatttctgctataaggtatttaacaaatggaaaaattcactgatgaaaaaagttaatgttcatcgatagaggtaccatgccaagtgctttatgctactcatctcactaaatctcataacggccctttgtggaggctctgtcataacctcagtctgaagaagagtacactgaagcagagtttaggtatcttattcagggttgtgccagcttggaagctatgctgatggaatatgaactcacatagtctttattccagaatccatactgtttttttaaatatgtaagtttaaggggtacaagtgtggttttgttacgtggatatattgcatggtgatgaagtctgggctttcagtgtaaccatcatttgaatagtgtatattgtaaccattaagtaatttctcatccctcactccctcccacctgcccacccttctccagtggtctccaaaggtctccagtgactattattcctctctgtttccatatgtacatattatttagctcccatttataagtgaaaatatgtgatattggactttctgtgtctgagttgtttcacttaagataatggccttcagttccatccatgctactgctaaagacacgatttcatgttaaacacgtgaactcacctgcttccatcaacttacatgattttcagacccttcggtcaacttggtgtcatagagtatgtattaaaactcagcttgatcttagattatttatatgaagatcaaacaattttaaatattatggatttgagtgagaaatttaaattattccaccgtgattacagattaattgtacaatcttcattaatcacatatctcggctcactgcaacctctgcctcctgagttaagtgattctcctgtctcagccccccgagtagagtaactgggactacaggtgcacactaccacaacaggataatttttgtatttttagtggagatggggttttgcaacattggctaggctggttttgaactcctggcctcatgtgatccacctgccttgacctcccaaagtgctgggattacaggcatgagccaccgcacctgaccctattagtaaatattttaacaggcagtcataagatagcagaatgtatggaaattaaatgatagttcagcaaattcaattaagtaagcatttcaagacttcttgctatcctctagaaactttgctatactagggattaaaagatgaatataataattccttttctccccctaaattttagtttagcataagattatatgatactgtgtaagttctattcttagttcttttaaaacgagacttggatagctagtactaattcattcagtgaatatttgtggagcactgctatatttgaagcatggttttcattgttggaaatacttggtggacaaaatggacgcatattactgttcttgtggaacttatattcttgtggagggggagacaaacagtaaacaaatatagtatgtcagatagtaataagtgcaaggagaaaaaccaaataaagcagggaggaggataggaagtgcttgcgggcagggtaggtaggtgaagggacagctgtggttttttgtttttttttttgtttgtttgttttttgagatggagtctcactctgtcccccaagctggagtgcagtggcgcgagctcggctcactgcaagctccacctcccgggttcacgccattctcctgcctcagcctcccgagtagctgggactacaggcacccgccaccacgcccggctaattttttgtatttttagtagagacggggtttcaccatgttagcggggatggtctcaatctcctgaccttgtgatctgcctgcctcggcctcccaagctgcggtttttggagagtagttagggaggcctcactgagaagatgttgtgcaaagactcaagagaggcgaggaagtgagccatgtggatatctggggaaagtgggttttaattagagggaacagcaaccgtgaggtggtagcatgcagggtaggttcaaggaatagaaaggatgtcagcgttggtggtagtgatgggcagaacaaggcagaaataaggctgcaaaggtaatcagcatgggcatgtgagggtggaggaatacatattgtgtagaggtttccagaccattgtaaagcttaaactttttctcttagtgacatgtaaagcaattggaggcttttgagcttaggtattgtgtgatgtgactaaagtttttaaataacccactctggcttttattttgaaaatagactatgatgacaagagcagagactggctattttaactatccagatgaaaaatcaacaaagtagcattggaggtagtaagtcgttgcattggggttaatctaagatgacttgctaatgtatgggttatggattatgatagaaagaaaggtgtcagacataacttcaaggtctttgtgcagagaaatgggaaggatggagttggcattgactgagataggatgactggggaagaacaggttggggttgcagatcaggaggtcagttttagacatggtggcagagacattgctgtgttttcaccaaccctctttcatacccctcttttctggtgcatctcagtttccttacagttcagcagggccatataactagtgctggcctgtgaaatgtaaggagaagtgataatgttgatcattttgagaatgaggcagtgagaagccccagaatgattctttggtctctgtgttctcctagggtgatgaaggaggaggaggcctcagggaaacaatggggatccccagttatgaagaacgctggattgctagtgcactgcatggggggcacattctgaggggagtgttaaccaactaagctaattaaacgcatgtgaaattttactttctcttaggcatatgcatcagatgtaaccattgggaactcagggaaatgagttgaacttgaaatataaatttgggaggcttcaagatatcaatggtatttcaaaacattatatttgttgaaatctccaaaagaataagtctagctagggacaaattaaaaggactaagccctgggcaccccaggttagagagcaaggagatgctagtttacatagatagctgcttgatcctaatacccataagcaatgagttatgtcatagactgtatgtggccacattcagtgaattttcctccgaggtgcttctttccaaatttccaaaaacacatatccaggtcactgattactggattcacctctatcatagcagaaagagcaccatacagctatgaataggtattgagacagtattcacttatttgagatttccatcaaagtaaccaaaatcatgaaatatttagaagaaaattcagtcaacaaatagttttcattttttcattgctttgtttgtagatgtgatgtttctaggcacttcaggttaggaaagaattaatctaattagagttttttaacctcagcattgttgacattttgggccacaaaattctttgtagtgttggggaggggtgctgtcctgtacattgtgttatttagcagcatctctggtatctacccactagatgtcagtagcaccccccttcctttgttgtgacaataaaaaatgtcttgagacattgccaaatgtcttctctgtgtgtgtcggggggaccacagatctacattacatctgtttccaatgagtgttttatttatatacttactgcctgcaaaacatttgagaaagtttacctgaacatttaatataatatatactatgctatgatatattattgagggaactgcaatgaaaagccaaagacataatatagaatttaagcaagaaagcaaaataatattgcaaaggtaacagtactatgctatcagcatggaaaatgtgttcaataaaagacaagaaaggaattatagatagactaggttacaccttcaatgggtcttgaaaggcagaatgaatttgtatggacagaagagaacctatcctggcctggggaatatcaggcataaaagctcagaggtaccaaatataaggtgttaaggcctgcctaggactgtggctaggttagaattactgaatcagaagctctgtgggcatgaggttgtgaggcagtgggaaatctggctgagaagcactgagactagactgaaaaggactttgaagccaagctgagatgttttcgtgttcttgggtggccaccatgatgctcattactgcacaaaagacagagatgtataagataacagtcctgtatttctggactttctaacctggacataatggaaaattacatataatttttcggcaagagattaatatatttaaaatgatgttttaggaaaattaaccagatggtagggagattactgaaggtgggaaattgagagtacgagagatggcttaggggatcttgaaggagtttgcacatgaactccgaaaggcttaaaccaagtggccgggcacggtggctcatgcctgtaatcccagcactttgggaggccgaggtgggcggatcacctgaggtcaggtgaccagcctggccaacatggtgaaaccccatctctactaaaaatacgaaaattagccgggtgtgggggcacacacctgtaatcccagctactcgggaggctgaagtagaagaatcgctggaacccaggagtcggaggtggcagtgagccaagatcatgccactgcactgcactccagcctgggcgacagagtgagactccatttcaataaaaaaaaggcttaagtcagggattggcaatttttttgaaatgtgaaaacagctgtagacattaagtaaatgaatgggcatggttgtgttccagtaaaattttatttacaaaagcatgtggcaggtggtgggccatactttgacaacccctggcctaaacttatgacatgatggtggaaagaaagacatcgagtaaagaatcaataaaaggaacttgaaaataaagctttgctgaacttggtaactaaaccaaatagagttagtaggaggaagggcaaggggtggacttggaggaggaaaaagattatcctggagtttcaagcctagaagatgtcattaatagagtttaggagcaggtatcaatgacttaaggaaagagaaagatgtgtttgaattgaggacttttgagaacatgttaaactagcaattggtgaaggcctgctgggccatgggggagaagtctggaacttctccatgctcatgtgtggaacagaacagtattttccttgaggtgatgcttttaaggggagatggtatctgaggaagggcagaaggatgaagataaacataggagaccagcatgtttatgaagtgcaaggaggaagatgaactgtaagaaactgagaaaagatagggaaatgagaaccagtaggataatagtgtcttcagagccatggccaagctaactctcaggaggaattggtcattactgtctgacgttccaagaggtcaagcagaatggcactgagaaagagattgtttcatttcttgaccagaaggttactggtggcccttcaaaaggaagtcccaagagagttgtggtaagaaaaggcatcccagagattcaatttagagaagataatgaggaattaagagaaagtttgtaagtttaaagaagagacacagaacagcaattgaatggagcaccttcttccaagaggaggcttgcttgtgtttgatgacagattttgagaaggattcagtggagaatagacttcaagttgttgcagaagggatgatgaggttaatggagaacagaaagaaggtgaactagctggagaggagtgatacactctgactctggaaatttaagagaacgtggatatcctggaattttccaaagtgctcttccttaatgcattctttttacaggaggcaaatacctttgtccaggcaagatcaggaagtagatggaagcagggttagaattaagggcttaagtagtataaagaagatttgcagtagccattgttataaaaaaatagtaggtgatctcctagaactcaatacttggattattgagacatactcagttttcttttagcaaaatatctctcattgactaggacttaaggctatactgctagcattgaaaatttattttattttgtattgaaacaattaagaagataaaggggaaaaataaacacttctaagattaatgaatatgcctctcgttatctttggaagaaactatgtggcctgttttccttggatattagcatctccaacaaatagaagagcctatgttcataaatatgattgggtcaaatttgacaatagagtcagactaaagttagctgacattgactttaaaagtacttgcctcaaagtacttgaaattcggtggcttcatattttcttggattagggattgatttctctgctcagacttcttcagaatacaaataactttgtaagggctaatctgttaaaaagtttgtgttagctctcttggcaagagctaggatagcagggtagaaggagaactgcgaaaacaaacaaacaaaggagagttaaaaatgaaagggcaaacactccctacttactttgctttgctttcattattttgcctaaactgttgtggttgacacacgtggacagtagattataactagattctgagccccagatgcccacagctaacacttatgtaatgctcattgtgtgccatgggctaagtgctataaatgaatgtatatataatataaatgcattaactcatttaattctcatgacgatttgatgaggtgagctctatctcccagtttctagatgagggattgcccaacttgtgggtaggggagcagaatcttaccccgggaagtgtggcttgagagttggcaattgtaactgctggacagatttgttagaaaaataccttatttatttttcagtcctccaccctgacttgtgccttcatcaaaaaagtttgaaatgttttcagttcatacataagaagaaaaaaatttgatatgggtttattttcccctattaacattttctttgatttacagtctgttgtatcagacatgatgtgaaaggggtagacaacaacctagactatatttatttttcaatctgctaatgtgaagaaaaaaggggaggagttaggtgagatttggagtctgtaatggtttttctacaccagggtttttagggagattctaaatagagcacactctcatttgtttcaattaaatgccttgcccacttaatctccttttcgtattgccatgcttgatacagagggctccaggactagtttactaggtggtaggacctttagagattatgctgtccaaatcctttacgttagtttaacagatgcaaaaatggagactgaaagggtttaagcagagtgagaacattaacaacaacactttgttttgaaatgaggttttttgattcgttccaaccttcacacaaatttgaaagcatacttttgtgtaagtttcttaatatttggaaattaacatcagccccagcctccctgaaaactgtggtgagttattactatatataaagattaatgaaataattgcttttgagtttctactacaattggtggatcagggtcatctaaaaaaactcaagcaaaggtgtgaaaactttattttgcatgtttgtttttctcctagttattatttactgctcaatcagcattaacagaagaaaaataatttggctgctaagttactgtagtataggatttcaaaggaccttattagagagagatgtaatttcatcccctgggccttttatgtaacagagtgcatatttgggttacttcattttgacccggaattaaacaatagacttaagttgtattcaggaagttaaaatatttattcaacaaagatttaaaagcattttacagttatttccaaattcctagggatatctttatagctttattttcttattttagctaactcttgtctgaatagctccttcatcaaagaattttctgtttatttcataaatcttataagatgtatgtatttgataactactgcatataatcagtgttttttaaataaacatctttggcaaattgatataaacatgattttatatattgcttaactacatatataggccatagtttcgttatggtgctcagtaatgaaaaatatggagtagatgttacaagtggtaataaagctaatattaaagaaagtaaggctgaagattcctaagttcaaaataaaaaggctttgcagcttcagttttaggagcgttttcataaaatgaaagaggcaaatagtgccaaggtgctaccaactccagcttcctagcaatgcaaggattcttcctttagccacttataaaagtggtcacctcagctcactctctgtaggactgactgcttcccctttcttctgtgatttgcatagtattatccacattgattcatgtttggggtgattgtttccatcctcactgtgagattatgaacttcttattttctttccatgaaataaagttttcattggctgggatcccagctagactgatttttttttccttttttcgtttgtttgcttaatattttagattttgaaagtccttcaaaattaaagtacttgcatactatcctagggcaataatttgtgactgaaaatctttgtgaaaaatcctaagaggtatttaaaagtttatgttaagtaagttgttatcagtggttaagaggcctttccagagtgcttgtccactggacatttatatggaacttactgtaatagcatctgcttaatttttggatgatgcagacacattccgttgcacaaaattactctgcaaaagtaagtcaggaatcactcatgataatttcaggttatgcatacttattttagaatgttgaatcaatttgctaatcttcaaaggagtaaattttacaccaattgcgtctggtttaataggcttaatattttcataatgccagagtaatttttcttcttagttttattcttgcataattgttggaattatatgtacattgttttaattcttgagcccttaatatcaaatagcttgagcattaagggaattaattgaaggccttaggatatgggtgtttattacatgaacatggattgaatgttgatgttgggattttaaacgggaagttgctttgctgttagattccatcacattgaatgtatcaacatttatacaaggaaattgggaagaaatgtactctcagtccctggttgctctttgtttgtgaaaagtaacaaatttaggagttgatctaaaaggaaaatttttgtgttaatgtttttaaaacattaaagcatctttaaacaatatttttgtctcagatgtctagatgcatatttatacccccagaaatttgaataagtctgtttaaatatttttgtccttttcctgaatgtcaaccctttggcagagtgtttatatgttaaagcttgaagagcaagtagagatcacacagtccaactctgtcatttcatagatgtaattagagtaaactaaggatcagtctgagtataatgaagtataaaaaagaacttgacactcactgtcacacaacacagttgatagaaatttgttggtcattcgttggccactggtcatcttccccccaggggtctctggcttgcaatgtattcccgtggtctctttctctgatcccctcttggatctcctcctcttccttaggtgttttgaatttctcttctttgtcttcagcatcctcaattcctcctacttcattttgcagaaatttagctgtctttgttatgccctcaccatccaatgtatgcccagataactgtgcctttttcatccaaagatccactaaaagatgacagtgtttttattttttatgagacacatcacctgcagaatgatttttttttctattacagttgacacatagagatgcattttaattcacatgtagacaaacatttgatgaattcctttttttatatatatacttgggaaataggtaaattttctgctatcaataatctctcagaaacacttgatgactgaatactatagttaaaaatcatgaatgagaataagaaaggcaacatagtattttaggagggacattatcccactcattgaattaccatataacctgtcatagcaccttaactgctctgaacctctttcctcgtctataaccttcatcctggcctgcactggagtcagagttaaactgtgccttgtaaaacataaagttctttataaatataacagttagagcctgggcgcatgcctgtaatcccagcattttgggaggctgaggccaggggatttcttgagcccaggggttcgacaccagcctgggcaacatggtgaaaccccatctctacaaaaacatacaaaaattagacaggcatggtggcatgcacctgtagtaccagctacttgggaggctgaggttggaggatcacctgagccctggaagtggaggctttggaagtggaggctgtggtgagttgtgatcaagccactgcactccatcctcggagacaggaatgttttatatatataaatatagatattattttacatatatatattacatatatatataaaattcctgtgctccctagggtgattttatatatatatatatatatatatatatatatatatatataattctattctatatataacattctactgtagatatatattttaaaaaactgttagatattatgatattatgagttgcgacagtgatgatgatcatcatctcaactaggcaggtctctttttccctggcaaactattcacaggtaggctactcacaggcttatcctcctaagtagtgtgtttatatgtactcgaaataactacataggacacaatgtatatgctttagtccactgaggaaatataaagaaaaaggatattcttggtctcttgctctttctgttttttttttgttttgtttttgttttttttgtttttttttttaagagacagtcttacttcattgcccaggctggagtgcaatggctgttcacagacgcagttataactcctgggttcaggtggtcgtcccgcctcagcctccttagtagctggtgtgtgtcaccactctgctcagtctcttgccctttgtgttgtcattcaacatcgaaatacatttcttcttctttttttaattttattttgaaatggagtcttgctctgtcacccaagctggagtgcagtggtgcgatctcgattcactgcaacctcctcgtcctgagctcaagtgattctcctgcctcagcctccagagtacctgggactacaggcatgaaccaccatgcgtggctaatttttgttattttattttatttttttagtagagatggggtttcaccacgttggccagacttgtctcaagctcctaacctcaagtaatccacctgcctcggcctcccaaagtgctgggattacaggtgcaagccaccacagccggccaatttcttcttattgaaacgtagatgtcaacgttgcatttgttagatatttcttaactttctctcttatttgagtatctaacatattgtatttcttttgctctagtgtaatatagctaaattgctagactctgaattaatatcaataaacatttatatcacacaagatagtgttatactaaatatatttaaatgttttaaatggtttcaaagttaatattttgagtaatatgctatggatggaaagcttattcatgaccagtataagttatacaaaattaatgtttcaaaggatgtctgaatttcaaaaggactcttcaaatgatcagctccataattatatttttcatctaattttatgttcaagtgctcccctagagtggtgataagtgaaatcatcgattaatgctaggactctaagctgacctagaaaaactacatttgaagctgtattcaaagaatttcatttagccttactaggacaactgtcattcttatgtaacattttaaagttttgcattgagattgggcttgaaataaatgcctgcatttgctcagagactgttaaggagggagatagagtaggaagttgtctccagaaataaaatgataatgaaacctattgtttatcgagtacctactaattataaaccacagtgattatatatgacataatttttcatagatactaaatatatgtatgtatatttgcaatttttatccttgcaaaaatgacaagtataattggggtatttacattctctttttaaagacaggacaataaaatctagtaaggttaggattttgcccaaactcatatagaaattagcaaacatgagatccaaatccaggtctgtcagaccgcagggtctttgtttactctgctatatcatatttattattcattgatggatgtattaaacaaagattaattcagcccttcccattgccaggaactgttatagatgctgaaaatactgcagtgaaaaaagacaccaagttcttgctttcaaataatttatattctagtgaaggaaatagaatatacaaatatgtctagtggcattaagtgctacagagaaaagagcaaagcagtgtaacaggatcgaaagtgatggtacaagaggtatttgtactgtattgctgtggaggtcggtgatcagggaaggctccttctgtaaaactgacatttgagcacaggcctgaaggaagtaaaagagtgagcttatgggaatacatgaggaaacagcctacaagcaaaaggaggagctcatgcagtgctcctcaagtggggacttggtggcatgatcatggaacagaaatgaggctagtgtatctgaagcagagtgagtgttggggagagagtagagcacaaggtccaagaggtggtgtggtgtcaaaacctgtagaccttggtcaggacgttggcttttactctgagggagacgtcaagtcactggaaggctttgggcaaatgtctgacatagtgtgccttgtgttttagatggagaatagactgtaggacaatggggaaggatccaggagaccagttgggaggctgttgcagtagttcaggcatgagatgatggtggtggcttgagttgggttgtacttgtggaagtaatgaggagtggtagaattctggatctatttcagaggtagagccaaccagataagctgatggtggatgtggggtctgaggaaaaggaagggtcagtaatgactgtcagtttttgaccctaagtattatgatgagaagcttttcgggagggaagatgggaaaaaaaagagttcagtttttgtatgatgtggggtaggttttttttctcctctatatggcaaacgattcccagcacatttcttagcctttctcctttttcattcatcatctatttttttatttatcatctatcaggtgtccatttatgtccatgaaaataggtttaccaatcgtcttgttcaaagcttccatcttactgatctttatcacttgacctgtaaaatattttgttgttgtcattgaatctatcaactagcaagacaggtgtgttaaatttcccactgtgatggtggatttatcagtttatcttacaattctattgttgttatatacataattgaaattatgatactggatgcatgcactaattcttgcattaattctttctggtgaattcatgcattaattcttcctggtgaattaaattttcctcaatatgtaatgctctttatcaaatactctttgttttaaggtcctttttgttttttatgaatgtgtctacacacatgatttctttagatagcatttgtgtttctgtttttgagctcctattgtcttgaactttatttgtgggaatccctggggtctgaattcagattcacttttgtcagacacttggaaaccctatcatctggggttagtttaaagtttgatttaggacttgtttggcttcacagatagcatgaattttgatcccaaattgaatattgcgggcttgtggttaggaattgtcaggaacagtgttttcttcttttcctcctccctctgagccaaggtcaaaacaggcttgtatccccatcattgccctttgcaagatggggcttttctggtttatcactgagggtgcagcccttcagggaatccactgtgatgtgaccatcccacttttaatatctgaagttggactcctggttttgccttactaacacccaagtttgagttcagggcaaacacagcctgtgcttgctaatcctacagaattttcacgtcttttcctttttgttctctgagatattccttactttcctgccatccccaccaggcattaaaagatgcttcatttttgtataatatttttcatctgttgtaccaacaaaggtttctctgaacatctgattcacagtagttcttgctgacatgcaacgttgtaaacataggagagtttgaagccaaacctagcactgtggtatctgttcaccagctatgtggggtggatattttcaggttgcatgaagatcctgttcttcacaaccattcactcagtggttttagcctccactaatgattgttgcctgaattacttattacaccgaggctacaatatggtgattttcaaattctattatttcttttacatttattagttgcctttttttctgtaaagaaaagagtttttccccagcatctctttacttttttcttgtcttgtgttctcatggatcattttttaatttgacgtgttatacttctttactatgataattattttgatactcaaattgtcttgaattttagggtggaagcccctgaagccaactcctgtgcccttttgctgtgcttcactaaagtctttgagtctatctttgctttttggcagaataagatgtcccaggctggctttatactttgtctgctccagatctggaagcagccaatttttctgacgaatccggcttcttttgatgagaagcgacattcagaaaccaaaattggagttctggatgtacctgttcctacaagtgtcattgtatttgggcccttttagtggaaagaacaagaaaatctgtgtgtgtgtgtatgtatatgtgtgtgtgtttaaatcataagtctgtattggtattctcaactgagagtttattttctctgaaaaagtagtgtcagcaattatagcatagaggtaaacaatcccagtttttcgatgtacttttggctgtttgaccttggacaatttacttaatttcactgtctttcttagtttccgcatctccaaaatcagaataataaaagtacctcaagcatagttgtgagaaataagtgtgtggatttatataaagtgctcacagcagttcctggctcagaggatatacttttaaattatttgctaaaaatatgaggtcggaccgtgtgaaactgccactatttgactgtctttaacctacagaaataatttcatgcggttcatcctgataaatgtgaacaccttcattgaccgaatgccttttaggggcagcaccctttgctttaaggtccattttgtttgtatccacagctctgctgtggataaagataagtaaaagagtctctgccttgaagaagctgagagattaataatagagctataaataaagtttcctagcactgtagtgctttctgcctgtggtgggggaatgaagcaggactcttcttaggttaccacaagcgacttgggggaaggagacaaaatctggcagtctgctgaacagggcggaattttagattgaagaccctagaattaaggtgggaactccaatgggatgtcctcagggagtcacttaggaaaatgatgaaccacgtgtgtgcaataatgtgtgcaatttgacacacagttttaatgcagacaaaaatctttaataatcatgaagctatttccataatatgaagaaatttaatatatgttaaaattctatgtatttctttgttgttttccttttttagaattttctgtgtgtctttcttatgtgccggaaacccaacctcagagcagtattttcaccattgagatggtgccattaaacacttaaaatcggctgggcctgtgatcccaggactttgggaggccgaggtgggtggatcacgaggtcaggagatcgagaccatcctagctaacacggtgaaaccccgtctctactaaaaatacacaaaaattagccaggtgtagtggcgggcgcctgtggtcccagctactcgggaggctgaggcaggagaatggcgtgaacccgggaggcggagcttgtagtgatccgagatcgcgccactgcactccagcctgggcaacagagtgagactctgtctcaaaaaaataaaataaaataaaataaaataaaataaaataaaataaaataaaataaaaacacttaaaatcaagctaatgtgattatccttcacaatagcactgcgattagaagtttatatgatgatcatgttaagatgaacatggggattggggtgctaactttatatatatacacacacacttatatacatatacatatgtatatataaagatctctatctatctatctatctatctatctatctatatatatatatatcttttgactaccccacaacccaactactaatagcctactgttgaccagaaatcttactgataacataaactgttcgttaacacattttttggtgttacgtatattatgtactgtatgtaagctaaagaaaagaaaatattaagaaaatcataaggaagagcaagtagatttactgttcattcagtgaaaatgtattattgtaaaggtcttcatcttcataatcttcacgttgaataggctgaggaggaggagaaagaggaggggttggtcttgctgtctcggggatggcagagatgaaagaaaatccacaagtgagtggacccacacaatttaaactcatgttgttcaagggtccactgtagtttattccatgattctgtcatcatctactgttaaatattaatgtaatgagagtaacataaaattaacacaactatgtggctgaagatacctgatcttccttcagaagttagtttcttttactggccagtatttagtctctgtattaatcacaaatatttgtacaaaatgtattgttgttgttattattattattattattagagatggagtcttgccctgtcaccaggctggagtgcagtggcacaatctcagctcactgcaacctctgcctcctgggttcaagcaactcccctgcctcagcctcccgagttggtgggactacaggcgtgtgctaccatgcctggctaatttttcgtattttagtagagacagggtttcaccattttgggcaggcaaaatgtattataactttgtttcttgtaggcatgttgcattcttatactggctgtaattattttcagaagtgctttaattgatgatagtgtcagaatggaggaatattatgagactcttctcatgaagtgtcttttataacttttaatgagcgcaatagttagatggttaccacagctggtctgcttccctactgtattctatgtgtccagttattttcccagtccttatgaaattaaaagcaaagtttttgcaaagtactaagaaacatcttgctaaaggccaactctagccaggccttgacttttcgtatcatgtgtttcatattttacacactttgactgtaatgagactggcatatccatgtgagtcacgctgatgaacaggatgtgtgtttgtatttacgttctgtatgagggttttttggaatatgtgaattattataatcataagagtatattaaaccatcttgataagaaattttaaacatgtaaactattcattcaaaaaaacatttacagagaatttccttaaggaaaacctgatgaatggatgtttatcttttaaacattatatgattgtattaataacacatgatgaaaattctatccactgttatctttggctggtgagtttttcctttgtgtacattctgaatttccaggtttttcccttgatgcaccagtatgacttttatgattatcaagattttagtggattcccggatgccttttatttggtgaagaaaacaatgttctaatacaaagtgaatgaaaatagctgcacatatgatattaatagccagcatttatcaaggaattactgcacattaagcatgtgttaagtattctacatgtgttacttaatttaatattcacacaattgtataaagaagatgctatttttatttctattttatagggaaattggaacttggtgctgtttaataacatgtacaatatgagagctagtaagtatcggaactgggatttggacctaccttcagaactcaagctcttatctactgtgtcacacttgatttattctgatggttttcattgatgctttcatttatgtgacatgtttttgccagtttgcattttgaaggaggaagcgctgggcttcaggagcagaaggatgaatgtgacatagcccatgctctgaaggagcttgcagtctacttgattagacggtcatggagctgtgggcacatctgcctgggacgaagagagaggaagtatcaggttgacttcttggagagtgatatttaggcttagctggtatagaaagggagaagggtattccagatattcggggaagtgaggacagaatttgcaaagcagcagtttttgcaaatgactcagaaaccagattttgaaaagccttggatgcctagtgaaggagttctgggttacgcagtgtgaacactggggaaccattaaaagaatgtaagcaataatagggtgggatttccttatctgttttattttttgctctttttttttaaataaataaataaaccaaagatttctctagcaatggtgtagaagttctgttttagagtggagagtttccagttaggaagatcatattgaaggctgtctgtcaggagtccaagatcaaagtccttaggggataggaagagatgaaaaggtttaagtgcctgggatttcaatctaatactaaagttgtacaaaaattcataacacttttggcagtatattcattaataatcgtttctcaattgaatgtgtttggaacttattcagccagaagctttgatactctagaaacaaaattgtttatagcaaacaatccgttatacttgtgtttttagcctagctcttagcttctgcaatgaagttgagacttttaagaaaatatatacacagtacacacaaacacacacatgcttacacaacaacttactatttatcagcttacactaagtctcaaattggtataagttttaaaagttaagatcttaaatcttcatgaacagatgatagtgttattataatgtgacaataataaatttatttttattaaatgaaaatctttttgaaaagttcgtagaattttaaatgtttgctgtagtcaagaaatattatattagcaatatcaaccacagataaaagttttatattatttgcctaatccaggggtttgcagatttttttctttaacaagacagaaagtaaatatttcaggcttcacgggccatgcagtctcagttgcagctgcttaactctgctattgaaggatgaaaacatccacagccaatatgttaatgaatgaatgttactgtgttctaataaaactttatttccaaacgcaaatgacaggatggatttggtatatgggcagttatttgctctatagttttgtttgtttacttgttcttgattcttaattttataatgatgcaggcttacactgaacaatactgtatttggttctagtagatgcttcttttaaccatattggtattcagtgtattgtcggtgatgcttttccccttagtgacatacagtaattttatatttggtaggactagatacattaatttatttgttttggcctttatgtcttcttgtcatttggatttcatggtgacttgactgtggtttgttctttgtaattatttcagatattcacttaactagagaggttgttctgtctatcagaaggctgattggaggcatgtgctggagctagaccagtgttttgcagccagtggtatgaacctggagagaccaaagtttatagcctctctctccactgcctccaagatagtgactgtgactgggctcctttctttccttcgtttctgccttcctctatttctggggttagttgtagatattttgaatactgatacacagtaaaattttagtttagaaacacaaaattttgggggttcatttaattgatggaaaagagaatatttcactttatcctttaatacttggtttaggaataatatttgttctctgtaataaaaatcacaaatgtgacaatataacagtttctcctactttaaactcacttgattcatgccatctttccttattgcctcagcctgaagtggtatctcatgtgccatggttccgttagtactttgttcatatcactcctatgaaatttctgtggcactaaatttaatatgagattattagctcctggctggaggtgaaggtggaggtggagtggtgggtacaggggtcagacagggaagacttgcctcctttatcttcagccgttcctggagtctaatgcagtgcttcacatgcagaaggttcagatcagtatgtgatgatggaatgaatggatagatattccacttgaaggaacaagggtgatgagtatgatattattgctagcagtaatttccttcatataccttaaacacataaaacttcctagtgggtttttgtggccatcatttagctcacccttaactaagcatcaatcctgtcctaagtattattttagattctggggatacatactcaattacttaattatctagtggggaagacaaacattaacatattattttaataaaatgtggcaggtgcaactctgggagctgtgccggattctctggagtctagaggagggcaccccactctgtttcaggaagatggacagtcttttctgtctcttgcattcccagccaaatcctgaaatatgcatataggcaagctagggaaataacaggaagaagagcagaagaaaatttagactgaagggctggctttaacagaggtgggtgagacaatgaggtacagataaggattctcaagctgtgttgttggaggataagtctggagaaatacactggggtcagcttctggagggttatgtgtatcatggtaaggagacttgaactttatcttgagaggaagtagagcatacagtggtcaagtggccattgtagagttttacccttgcctctacattttctgaatgtcagacaccttctctgtttttctcacataaaatggggatacaaatgatctatctttcagggtttgtgtattcattcaacagtgaatttatgaatgcataccatgttccaggactctaggccttgggaatacagcactaaacgtgagataaatccttattccaaagagcttatatttctgagtgtggtggtgagatagataagaaataatatcagtgaaatatacagtctgttaaatggtgggtggaggataaagaatctgggactaggttaggagcgaggaagagggaaggctgcaattttaaataacattgtctggaaagaattatttgagatgacaattgaatgaagacctgaggacatggaggcccaattaattgagggtatttgagagaagagcgttttggagggaagagcaaatgcaaagaccccaaatttaatgcctgacatgtttgagaaatgcaaagagtcatgaaagttggggcagtgggagttatagtaagagatggggtcaggctagtagtgggggccagagtttgtaggtctttattggtcattattaggatttggagtgagataaagccactggagagttttgggcaagtaataacataatatgtttcataaggacaccttggctgctgttttgagaatagcctagctagggcaagggcacgaggaagaccggttacaaggccagtttttaggaatggagtggtgagaagtggtgcttgtaaggaattaaataacttgtaaatcacttcatatttaccgtgtgcctgccaaatggtaaggaattaataaatgtcagctagcgtttgactgttaaagcttctatctatgctcagcaaacagggtttaattcttttttaatctttgagtttcacttgtaagaataggaactttgctggctaagataataattgctatcatttagccaagagcattcatggtgagttagcattgtacagcctccttagcatgttttctcctaatcttctcactaacccaaggtggtagctctccttacaccaagatctatgctacatgtggtaccactggggaagttgaaggtattcaggaccacagaattggtaactggaaaccaggtctgtctttttcaactatgtccattagattgcatctgatattcaaaatttcaagtagattctatcaaagaataaaatatgtaaggaaagaagcatatgaaggacatgtctgttatatttctgccattattggtaactcttaagtaataatgattatctatgtgcttaatagatgtcattgtgtgtgtgtatttgttcttctaaatggtatgtttgatatgatcacagacgacattgtgccaaatgtaaccacttttcctggcggttttaatggtgttgcaagagtctgtgatgcttctggttccttttctgtccgatgtagacaatgcattcatattgcatgatgattagtacaaagtacaaacaaggagactagagaaaagagaggctaaatgaccaaagatacttaccgcggtaagtggcagagttcatttgagaattcaacagattggctccagagtccgtgttcttgctgagtagatcaccccaagggaaaactataggcatgtggtattagaagggaccttatagacagagctcttcattttgcatgtgaaaaactggggttctgataggtagaatctgtagccagtaactggtagagccagtactgtaatctgtgcctctcacttccattacctggcacagccttaatttgatcagctgtttttccagatgaaaagtgtattctccaagggaagaaacaaatactttctttattctcttaaagtttgtgagtggctgaatgttttaatgtgacgatttattaaaaactgaatctttaatttgaaaatcagatttgtatctttcagttgttttatatttaattatattgaaaaatcagcaaatttagattctctcatttgtttcatgttcatttatgatagctaacacttttaagaacctgatattgtagccatatttaaatagttatttctacaaatcaaatataaggaataaaatatgtcttatttataactcacataaatttaaatcatagaagaattgaacttgacagagatcctcagtaagaactttaaaaaaaataagtatcacatgtatttaatccatacttcccagtcattgcaacaaagcagggtatgatttatgtagaaggaaaacatttttttgttatttttgtagtggggcatttattgcacacacagtaagtgttataaccaaaacaaatatacaattgggacactgtgtttaaattatcttctgtcatgtactatataaacccacaatagtttgaaatgtatttaagccaccatctgggacaaagcaaaagctgttccaggatccagcacatgatgaatgtgaggcgagtttcattaggagcctcctagcacgtttctgcatcctgaatcgtttcttccacggtttgaaagaattccaacatccgtcttagctccctgaggttttgagtgtttttaatagatcatgaaattttttgtggcctcctaaagaactaaatcagtgaagttaaacattattgcatttagatttccttttaatctggagagataaaattcaagtttgtatcttttaaaagcttcagcagaaatgccccatctgttgggaaaaattcttactgggttttgacagtttgaaaggaataatttctcttttaaagatgactcaccttaattgagaacctattgccatgataacaggtagctatttaatcaagtacccattaagtgctatgggctggaggcagttcaagccttaatactaatctggcattctaatccagatttgactggctgtaaagtttatgtgagtgtttttctttcaggaaactgtttgttgccttatccctatataatgacaatagtacaagtagtgatagttactgaggccttaccatgtacaggcatggttctaagcactttctaaaaattatctcacttattcataagaacagtcttaagaagtagatacatttataattcccattttacccgtgaggaaattgaagatttgaaaggctgtagaacctgctggaggttacatggctagtgatatggtttgattgtgtcatgacccaaatcttatcttgaattgcagctccctcccataatccccgtgtgttgtgggagggacctgataggaggtgattgaatcatgggggtgggtttttcctgtgctgttctcatgatagaggataagtctcatgagatctgatggtttttataaagggcagttcccctgcacacgttctcttgcctgctgccatgtaagatgtgcctttgctcctcctttgccttccaccatgattgtgaggcctccccagccatgtggaactgtgagtcagttaaacttctttttctttatagattccccagtctcaggtgtttcttcatatttcttcattagtccatgaaaatggactaatacagctagtgagttctagccttctaatttaactttcatggcaatgcaggcattattcctgtttttacagatgatatagctaggatcagagaggttaaataactcacccaaatcacatatgtaataaatagaaaaaggagctgaatccatgacttctaattccaaattcagtaattaaaaaaaacttcaacttttattttagatttggggtacatgtgtaagtttgtgacatgggtatattgtttgatgcttaggtttggggtttgattgatccactcactcaggtagtaggcatagtactgtataattactttttcaatcattctctctccctccttcccctttctaatggctcccagtgtctgttgttaccatctttgtgtccatgagtacccagtgtgtagctcccacttgtaagtgacagtatttggttttactacttatgagatgcggtatttggttttctgttactgtgttaatttgcttaggattatggcctccagccgcatccatgttgctgcaaaggacatgattttgtcttttttatatctgcattgtatctcatggtatatgtgtaccacatttttttatccaaattcaataatttttaaaactaaaacatgttccttcattttcagcaattatatgtatttttatgcatagaaacttaaatacaactatgtaaaatattttaaatatgtttaatatataacttaaaaatttttcagagaggtatttttttgaattttttttatctaaattgaatcagagaggaagcaaggttgtgactgtggatatgacttttcataactaccaaactttctgtaggtggactagaatattcactttttaaaacaaaatatagcaaaaaatataacaaacacagaaaagcacttgaaatattgtgtttgaagagatttgtgtataattttgctatgctattgtccaatatgatacagtttcctttagtagttactttaaatatattttttattattaaaacaatataatattcttttataggttatgaaaaaaatccctaaatggtgaaattaaagatattgaagttgaggtttaatgcagaactgcacttgttttcagggaacaaatgtcaatatattaaagagtttaagtagtgtctgataatggttatatggtgatccagaagtacagtaatgattctcttaaaatattccattttaacttattaggcagttatggcttcttatatatgctataagcaaatgtggtggaaacatattttgttttcataaaaaatttctgttttaaagccttctagacattttcaaacaaatctttgttctctattctgattaaggcagtactattccaaaatatttaggagtggttttcattaatttgctattaatgttaccattctggcttctttcctcaataggagactaataaaaccatgtgccgcagtaagataatgtactaacatgccaacatgcctccttatttgaaaccagatctctttttctggtttgaaagttgtggatcttaaagttctctggcacaaggaaggcaagtgaggttagctatggagagaagattgcacaattaaaccccattgtaatgtacggtctgtctggcctagagcttcatcttattcttgtgattatgaaacagtattctgtcctgacgaagaaggtagtggggaaaagtttctgggatttgtaggagagtagaaaagaaagatatttgggaaggatatttttaaaaaagaagggtatctctatgatttgctgacaattaacaagtcttaaacagacaatcttctcttaatatgaggttttatgtgatgttgtgagggagcctggtattcacaccagtggcctcagttatcacacagcagcgaaagtgaggaggtcaattttacttatttggcaatttaacgtgaaagcactgggttgtgttttgataattcctgccatataggatggtcaataatttctacttgcaaaaggaaaggaagcctttttcttagcaaaatattggttggtgaacaggaggagatcttagtgccttgttatttcgcatctgtgtatttgtcatcttattctgacttgaaaactacttcactatctatatgattattttggattctgagtagttactcatttatctcccaccaatatgttattgcaacatgtgtcatctgttaccctccccacatcagctaatcatttgacatataattttaatataatatgctaggatacagatttagtttgaagtttaatatttttcttctagctttggtgagtgaattattaattgccattgtaaagaaaaatgtatcatataactgacatgagaaatgatcgtcaaatatttggtaatagttttttgaggacttacgaacttgaatatggcactaagatttggttaacagtatttccattgatttcaagacttcagtgactaaaagatatttggaggagtcttggggaaaactaaattgtgaaaaacgctgaaaagaaatttgtagcattgcagactgtagcagagttatatgatttagatgccttgtgtttagccaaatcaaaaatccaaaaatggatttctgaatactaaagtattaggataatttaacaacatgcctaccgttaatttcctaggaaacaacttcgtgtgttatgttttgaactttcctttgtattagatttctagaaggacttatattattcttgtttagtttccattttttagaatgtttcttttgatgatcctgtaactctcataagagctcagacttttcccctatacctatatttgaagttgtcttttgtctctctagtatttcaagcctaaacctaggtttcactattttcttcttgcttattccccacatacattctcttagtattaccattacccaggcttgtccattttatccctcagtctgttccaagcctgttcacacctttcctgttctactgccttcacatctgctactgccggcttgtgataatccctctctggaccatggtagttgcttctggcagtgtcccttccttcccctctttcctgtggcatctcttctttatacaatggaagtagtaatttttaaaatttaatttaatttttatttgtttatttatttatttttagacagggtctcatgctgttgcccaggctggattgcagtgatgcgatcacggctcactgcagcctctacctcccagacccaagcaatcctcccacctcagcctcccaagtagctgtgactacgggtgtatgccagcatgcctggctaattttttgtggagatgggagtctcccagtgttgcccaggctggtctcaaaaacctgggctcaagcactcctcttggttcagcctcccaaaatgctgagattacaggtctgagccactgcacccggcttaaattttagattagataacatctacctcacctccaaagcccttcagtagctagctccctgttggactctaaaaatcccaactccttagtgtggcctgcaaagcctgcatggtctgcttctcctggattgtaagctccacgaggacattcattatcattcaggaatgttcattaaaacacttgtgtccaacaccggcactgtgctgagtactgggacatagtaataaattaataaataagaatgagagaagaaaaagaaatggaaaacaggaacacctgtggactctagtcatggaacttcatctactggtagaatagatattatccacgtaattgcagaaagatctgttaaaagtacaacactgataaatgctgtgaagaataggttcatgattttatgaaagcaggtaacagtgaagtcttactgagatagtcatgactgaggtgatgagtgggagtacaggaggttggggtgtttgtgtgagtggggagaggagagaatatgcagggagggggttttctagaggaaaaatctgcatgttcaaagcctgtaggggcaggagcagactgtggctagaaggcaggagcattggcggttacctgctgttaaggatttctaataagctgaatgtggggaggggtgaggaaaagggaggaattagtaggcttttggttaagcacttgggtggatcttggcaaacattaacatttggagaggttcaagtcttggtggtacttgtgtgtgtgtgagagagaggcgcgtgcacacacacacacacacacacacacacagagataaagcaagagaatatgagatctaccttaggcaaatcacttaaccttttgaattgcagattcctatctcatttgaaaaacacacctaaaaatgcctaactcattatggtgaacgtcaaatggaattgtaaaaatacgttataaactgtgagttggtattggtatacatgtagatgattgtagttaactatttctcagatatttccttgtattcctcaatattggtttgcagccattcaagctggactatcattttgatattcttcctcaccatatagctttgctcaagactccaccctcaacaacaaagtaaatgttttaactggagctctaactctttaatagcttctttgcttccatcctctttttctgcttctaagaaatttaacttctcttcttagtttttttcttaccacccttctcattttttttttccattccttccatcttgggctatatttcactttcagaataggactttcttccatttccctgtagcactgagcctaagactatgcccttttatgtttcctttgcctttccttgttggggagaggggtttaggattaagtgtaggtgtatccacatctcctaagatcttttaagattatattttattttcattttcttcttagtaagcaggttctcttatttaaaacctctccccaaactcatgtcgataacattttattacttttttaaagatagaagttacatattggacttataaggtatgggagtgccagggtgtgggagagcttgctttgttaactttttggtttacaatgaaggagatgctcccttttattttggatagaaggacattgttcctcctcctgtggagtaggaaagtttgctccaagccaagcatgcctccttcctgaggactgaggaatagctatctgcctgtgtctattcttcacattgcctggagaaagttagaatagaggtggagattgagtatctagccacatgaagtgattcttactgtcattcatatttaacatttattgcctttattttggggaaaaatcatttagatggaagactcctctctttttaattttatctcccattccagctgatctacagtatattttctaggagaaaatagatttagatactaattaggataatttaatgaaatataaaatctgtgcgaggttagttgttgctattccaataggaaaatgaaactggattcagttatcttattgctaaaatatgctaaccaagattcagctattcagataactttagagctgtaaaatatattttataactctgttttcattctttcattaataaatacctgcagtaattccctggcttattttccttacacagttgttagagtaggactttgttttctgtcagtttcattcatttcattattgtatttaagctgactatagagaaatcatgttagcaaatgtatatgaattatttctccctttccatgaggatctttctgatgtcttattttttttctgagagaggctgtgcattgactttggttagaaatggtctgtcttttcaaaataacttctatgtaattaaaaatggctgaactctaatatgaagctgttatcttgaaagctatttctatatttaaaaaatcttattttaagttgtagaaagcttgtatgttttatgttagtatctgagtaaagctatcaatggaggtacatatgtggttaaagtgatgaaaatataactatggttaaatcattcatctgttgcaaattacttatgacttctgatacagattaaatgaaatgctaggaaaaaaatatagatgagaaaaatataagtagcaccgtatgtgccaatgtgtgactgtatgcttgtatatgttatacacaaaagttgttaacttctgcaaaactttgaccattttcagtttacattgttaattttcatttatttgatttagaaagaccatttaaatatgttcttaaacctagtgacaaaatgtgtggaaaaataatgagaaacatctcacagttaaatatttttgtttttagattagtcatgaatgtcttctttcctgtagtgtgaatggcagaaagttaactattcatggtgtaatttgtgtgaaaacatctatacttgcatatttatttttattattctattttatagaaagcattaattctccaacatggtaataaaacatgtttttccttcaggtacactggtgtaacttcccttctgcatgaaaatatttcagtttatcaaatcatacttttcaactaaaatttggttaacatagaaatgttacaaccataaaaataaagcgctagtattaaaaaaggttaattttgattttgatttttactgtctttattatctttaactgttaaaacatgtttagctcctaaaatatttaatagggtaatttaaattactgcatagcaccctccttttagtagctggtggtaggaactccaaaccaatggaaatttctagctgaaattggaatccatgaagaaccaaactcaggagccaccttgttttccttcctcatctaatatggatgtttaatttggggtggtgactgacttttcaatacaactttaagagaacattctatataaactgttgcgtctccccagctaccctcagagaaaaccacatgtctaagctgaggggaaattcacccgcatggaacggagccagtctcttggagaagaacatggttgatggaacccctgaacaataaagatctgtgattactgaagcggctcagaatttcatttttgaggccactaaatcactcttggcctgcacagctgtgtagtcaggggaggctgcagatattattcagggcagcaaccacagagtctgttttttatagaatattttcccagtgcttttggaaactcatcaagtgttaaaactcagtattttctgggaaagttgaagatttttctctccttttatggcaatttcaacctcaaggtgaaatatttctttaattatgtgtaaactttattttttaaatgagaaaattacattaaaattacaattttgtgggacattattaagcaagaggggaaaaatctaccgttatgtgaggttgggttgtcagatatttttaaattcagaaaatttgctgagaattctccttccaaaatggtaaaagaaaatgagtgcaagaaacgcgccactgatagaaaagtgggaagatactgaagcaatgttgggaacagaaatgatcagggtgactgaagcagagtcatagagccttgaaatagaagtaaaaatgtaaaccacggagacaaacaggaagcttgtggaagtgaatgatggtgagaaatgtgttctacagtaagatcatgaggtatatggattcttattttacattcattttaaaagcatttttccaactgctaagatttatattattggaaagatgttttgcatcttctaatagaaattcattttggaatagagtggcagccacagtgtgggaacttcagaaaagggggtgatgtaaaatcaacaggaacagagtgttttttgaagtgtcagcagcaaggtctggctaatgggagcaggctgtgacactaggcagcatgtactccagaaagcacttctgctttcccgtagggcatcccgcagaccctggctgggctcagcaccaccttctcattgatgtccctgtgtaggtgggtatacgatacgtatgctggtcaagaggctgtccctaattctcacttcagtaagctgaaaactccctaattctcttaaaagcctgaaaactttacgtttttctcagcaaatagttgatattagtgaaggcctatttagagatttctttgatattataaagtgtgctgatgatcatagaaaaaaaaataagctgcatttatttgttccctaaattcttaaatacaatcctcccaccgtgcataattacagtccccaaggtaaaaatctgacagttgtacttacataatagttacataatcagctacttaaataggttttagctattagctaaaataaataaatcctagagcagatttttctgggtagtgaagttgtctttttgtagctgttgagtactggagttctttttagataacctgatagtttagaaaattaatactgaagcaattaattgaaattgacttcaacctaccaattccacgactgtcgttttcttctttagttactttccattaattgactacgctacccctctttggccctgctgtacttgtacatggctatttattcatactgtgttttgcctctcattcatgccacaccaacagatgcataaagtgtccatctctctattttcctctgaaaacacgattttgatgcatctggcatggccttctttttccttgtgcatattagttgaaggtacgtgtgagctggagctagggctctcaccacttcctaactgccttgtgttggccgagaggtgctctggagccttccctgggtcctagtggcttactaggacctagtagtttcttcctttggcctgcatagaagcctagcttagcatagaagccttccttcggcctgcatagaagcaaagctttactcattttggaccattgaggcctcatgtgaattctttgtccattttggatcaaaaaaggaaatgacaaagagagacagaaatagaggttagagagacaagagtctacaccagatggtgttctcaccttcaaacagctgctcaaaactgtacagctccatatgttctcttggcttatcaaaaagtcagaaggctctgagacattaagaaagagatacttgtgggcagctcatttcaatagctcgtagcacaaacctgtaattaccacaggacaggtgtgtgcagatcttcatgatgctttgaagaatctcaagttcatttgagaacattgtcataatgacccatataaataaacagccaaagattaatttagctgccaggtaagcagtgaccaaattgagttttcagtaacaaacccagtcatcatttctcctgggtaagcactggaaccaacatacttatgattttattatgaattatttgtgtatgtaattaataataatgtatttctgctctctttgcttttatataggaactactcaactttaacacaattctaagttaatatttattcttgttatatcattatttggaaaatgtgcttagattaataagtgtaaaaataatcataaaataatacatctcaatgttctggagtaaaattgttattaagtgtatgaaataaaaaacaagttatgtcttctcttcatcgtgtcctttctattcccaaggtaataatcacctaatagttgggtatctgaatttctatatgatcattctcaatctcactctcaatcatctccccttttgtccatccatccagccccccgttcatctatccatccctagattcaccagttcccccatcctcccatgcccgcatccaggcatccacagattcacccatccctccatcccccggtgccccatccacccacccatccacccattccctcatccatccatccatcctccttccgttgatccatctacccacccatccatccaccctcttatacaagtgcagcctttatctcataatgaaacttgctttttaaatgtagttttgaagataaataatacatatttgtagaagtcacctgtaattgtttattgacctacaattcatggaaatgatgtcctagtgctttatttcttagcatgtagttatttagttgggactttttatttttgctgaagcatggcaatttgtgagcaatttaagtctctgccttgtggtaggggttggacccttctatgggtgtgactcttcttttttattgacttcgctgatgatagaacttaggggaaaattatgagaggaattaccacaagtgtagaatcggaattttaaatctgaaaaggcttggagattgttcagaatttgatgtaaacatttttctggcagtgaatgaactgttaaaactgtgatttgccggtggaggtaaacaaaaaccaaagactttttctttgcttgtcctaatttcagatcctagttcgcttggtgcctgtgtcttcatcttcgtatctattctgtcttcccatctgataaaacttgaggttggtttattctgtttccagatgctctttttattctgctacccagaaccctcttggttgaaaaagtaattttaagccattcttttttttttttttaatcatccagagtctcctgagctaatttagttccttcggcagctttgactgcccttttaaacatccagcatacctgagtgggttctgtgcctctatagatgttgctgacacatatcactcgtatttatttttgcctggtactctcatttgtatgtaagtgtgttcctaatgttgagtttccttgcaagtcctgaactgtgttttctactttcttcttccacttcctttgtactattcatggtttttacgcacgcactatattaatgcgatgatctgagcataaagtacgatttttctaaagctcatatgaatctgtggagttgtgtccaagcagtgaatctggttataggaggccacatttacagcatttctgcttaacagcctgtccagcaggcagctccgtgctgtctagtaagtcctcattctgatttgtatggtgtagatccatctgtgcagaataaaatttcatcccttctttccctctcccacctcttctatcagattttagacctcataaaacctctcttcactgctataagagaagagggaagaattcaaccccagggctttgctccaagctttgaggatattcacatggtgatagcttttaaaagatagagaatttcagaacaaggcataagccggaagttgtgactctgcatgcagaattagcagttagaatttagaggtacataagtatctgtaattgccaggaaactagatgagtaaaaatcttccctatggattgcgcctttttttcttacttcatagctctgtcagtttttactttgtgtattttgaggtcatgccacttatgcatacaagttcataattattacatcttggtggacttctctttgcttattggatagtgtccctttttttttttaccagtcttaatgattatttttaattaaacttttttatatcaacattgctgtatcagctttcttttggctagtattactttgatattctttctaaccccatgttctcgcttaaaaaaattgttttgcgtgattttgttttaagggtgtctcttctatgcagaatatgttttacttctgatttttttcttttcttttcttcttttttttttttgagatattgcttcactctgtcactcaggctggagcacagtggtacaatcagagctcactgcagcctcaatttcccagctcaagcgatcctcccatctcagccccccagagtagctgggactatcggtgtgtgccaccatgcctagctaatttttttgtatttttagtagagacagagttttgtagcccagctggtcttgaactcctgggctcaagcaatccacccacctcagccttccaaagtgttaggattacaggtgtgagccaccatgcccagcctacttcataattttaaaaatagctctgataatttctgtatttgaatagggagtttgttatttacatttattgtatttgtgtgtttgtatgtattcatattctattgagttttcgatgaccattccttttttttttttttgctcctttttttcccaacttcttgctttctattggattgttagggatttctgtttttcactttatttctctctgcttatttgaaagctatacagcatggttttctttctttagggatcactcttccactttactttttaaagatggataaattttatacatttaaaaaatttaatctgtatttatatcttcttcctgagtggaccttagcatgttataaatgctcactgaataattctcattgttaattagagtttggttttatttttttaaatgcaatgtacttacttattttagagtaaacagttaattaaatctatattttacttgtttttttttctcttttatcctgttgcattctcatggattcatttttctttttttgttattataaattcagggggtacatgtgaaggtttattatatagacatattgtgtaatgatgaggtctgggcttccagcatacccatcacccaaacagtgaacattgaacctaataggcaatttttcaatctttaccctcctttaaccctcccctctttttggatttcccatgtctattaatttcatctttatgttcatgtgtacccattgtttaggtcccacgtaaaagtaaaaacatgccgtatttgattttcggggttatttcacttaggataatgacctccagctccatctgtgttgctgcaggagacatgatttcactctcttttatggcctcctagtatcctgaactcatttttctacatactgaggtgtatcctcaagtaattgattcaacaatggtctaagggtgggtggctttcttttaattttagttttgaataatactttagctggacataacagtctagattacaaattgttttcttctcggcacttcaaagatgttatcctattgtcttccgcctaggacacaataaacaaaaatatttttgaatgaatgacacgttgttgctaataagaatctgctgtcagtctgtcggttggtcctttgttagtaatctgttttgtctccctagtagcttttgctacttacttaactttgtttatagtctattatgttgtatctaggtattgattcagctttatttgtcctgaatcctatgtaggacaaataaattcttgatgtcaagaattctaactttattaaaattctcaacttttatatctccaaatagggttgccttgtctttatgtgcagtccccttttctgcaatttttattagatagatgatggagcctctcagttgttctgtgctgtctgctcatttcagcagctttaaatttcctttctcatgttttaaatttataatgcttttatctcatgttctttttacatttctgccagttgttgtttgcagaagttttacttttgagggaggaagacaaggaactttcccccttactacctatttgaaaatcctaaacaaacttccataaaaataatttcatgtggagaaaaattcatgtttaaattggtcgatcttgttcactgtatttcttgggattagaatgtatttctgtgtgttttggaattttggtgtgctggcttgtatttgagttggaagattccagcaaaagtcttttctttcccccacagcatcttttcacattagatgaaattgagtatgagatgggaaaatgagcaccacacgagttcagtttcatggagtcatcctaggtgtgtcctctctactgtttgagctcccttaggctcatggtctcaggagccagattcttgcctgctctggacgtagaggcccgagggcccatggtctctcctctcttaccactttcccatttcctgcctaaagatgtttgccatatttaaaagatatcagttacagccctatgactttgtatattttcagattgtgtttatcattgctatggattttgagccagagggcttatgtcagtgatcaaatgtgtcatcttgtttggaagcaatttgactggaagttgaggctagacttggggaataactgctgagaaaaaatttaaaagggtaagcattgattattgatcaagtgactatatcatttaatattcacacccagatacctttgagagtgacagaagacattattagtagtgatataggattataagtgtaagccagagctattataggaatgtagaattgtgtagtcgttgtgctataaaaggactatcctctttaaatttttagaaaggaatatttatgattcaatatcacagttatttttattgatgtctgtatgctacctagagttacaagacagctttttctataagaaacacatgattattgtataaatataaaggccaagagtctttgaaaacatggagtatggagctgagaaagaaatgtgtgtatgagtaaatctgaaactgatgttagtcatgaagtttttcagcaatatgtaccattccctctttaaaatatttatagtattacatctcatagtagctgggcctaattttccaaaaaacagccagaggaagaatgtcagcagtaggagttaattttgaggtaaagttgatgggcacattgtatcataagactatgtcttaactactataacctactataattcctaataaatcttgtaaataagcatatatgagaaatttatagtgtttatgtcttgcaaatacagaatgaaccaatatatagaatttgaattacctctagtaaaagtaaaggttcatgttatgcaaacacatttcagactttctatgagttgcaaaacttacatgtgtaccacacctgcctacatgttttgtcactggtgagatgtgacaaaatgatctctacttaccctatctttgaaagcccatgattttagtgattgtttctaaatataaatattatcagcttctcaagacaaaattcattttattctctatatctatgtctatatcagtataaaatccgtgtgtgtgtgtgtgtgtgtgtttgtgtgtgtgtgtgaggatcaaaaactcatgatgaataacaggctttaaatttttttattcgttagcatatttccccaagccaacataattcttgtttttctctttttagaataagctcttgccatcatgttggtcagatagaagaagagccatctcatcttttgtttgttagagggtttcaattatttaataagagcaagtaattaagacattaagaattaaagtggctccaagaagagctctgagactcagaactgcagagtgttgcctaggtttctaagtttggatttcaacagaagtgtgaaaagtttctgctgctgcttccttcaaaaatttttaataagatgtagtattttgacatattaatctgaattgagactgaaaaattgcgtaaactttggttaatcagatcgattgtcaaatccagctatattgctgtttcattttctactaaaatttgaattattcatatcaccaaatagctcagcaaaaatgtttagtttttgcatatatgtaataaaaatagttgataatgaggttctaggggaagcaaggattttgggtgtgtgtacatatgtgtgagagaatgagagaaggaataggaatgtatcaggatagaatgcttctaaaacctcattttccagtaacaggtttgatatactactgtggtcaaaaataataaagaaactataatagctatttctgtattgttataaatgctgaagagtcctgctctcagattacaagatacgcctcaaggaagaagtagtacaaacacaaggttcttaatataggtccataagaagtggcatgatgtaatatctggatatctgggtggtcactcttatttctcacacaataagaatgaactttttgggattaaaactcactctccattaaatttttagaatttccatctccatgtgaagaagtttgagtattattgatttcttcttctttggaaaatcagcccttcagcttttcaaagacaacttgaaaattggacaccaattcatatataatgcatatatattctccactgctgtccaaacagtcattttgtagcttcatagtagaaaatatagttaaaattcttaaatgagggaaagcacatgaattattatatagtgcatctctttatatgaccaaaaattatgctgtattctagcaatctggttatatttttgaatagtatccataattacaatatttatttacaattccatttaagctcctgtaattttgacggaaatggaagaaatttagaatcttggaagatcttaaacttctggttttgttggttgtttgttttctggggttacttttggccaggaggcagagagaagtgttatatagtacttgattcagcaacattctattttcaagtatttattattttaaaagtttttttatgtattatgtacaatatataactgcaattcctattttggttttatgaattattttttctatttttataaagtaaatctataaagttattctattatgtgtctgaaatattgagaaagaggatccttgtaaaagtggaaagataggagaccaataccaggttgtttctctgatccaggctggttctcagcatagctcaggaccctcagtaaatgcctgttgagtggaaggttgcatgaacttcatcgagtatggattcttaaaaataataataaattaaaagaaagcttttcattgggaaattggtatttcaaaaaatcagattaataaatccagtcttttctttcaagttttgttagtttttcttgaaaatttatggagtatatttcagaagctattttttttttttttttttttttttgagacagagtctcgccctgtcccccagactggagtgcagtggcacaatctcggttcactgcaagctccacctcctgggttcacaccattttcctgtctcagcttcctgagtagctgggactacagtcgcccaccaccacgcctggctaattttgtgtatttttagtaaagacggggtttcaccgtgttagccaggatggtctcaatctcctgaccttgtgatccgcccgcttttacttgagccactgtgcccagcctcaaaaactattttgatagaggtggtgtaatcttctaaagccagctggcacatttataggatgccaggctagcccatcagatgaagtgtccattgtggcagccacagatccacatcagatgggaagagtgtgatcagtgctgcatgcatgtttaatcaggtaacaatgggagctgtacaggaagaggattattattccaagaagaaaggacctatatcatcttgtggaaggtaaacgtatccattgggagggaaaaataatatatacatagggagagagaactgaaatatcatcttattggtgtaaaccattttgttagtatcagcattattattgttgatattcacatctgtatgatccatctgatcaggatgggttagctgtcaagtcatcaagattgactttatgacatttttattatctgtcctttcaaaccttatggttctaaattttcactgtttgcatgcactttcatgtataaatctgcatctggaaagactgatatttctctaaggcaggggtcagcagatgttttttgtaaggggcaaggtagtaaatgttttggaccatagagccatggctgcagttacccagctctactgttgcaatgaaaaagcagccatccacgagatgcaaatgaaaggtgtagctgttagctttctgttggctcagctgcacaaataggagcatgaaacaggctgtagtttgccagccactccttgccacatcctcctctgtcattagctacccgtcttggggttcgttctgctgtatgctgtatctacaggttcctgtttgttcctgtttggtggtctgcctctttgcttcttatgcctcactgaccttcttgtaggagaagcttgtggggacttccatacatctttcaagttcagatttgggaagatgtgtgtcacctctgcccttctttcctgttcctcacatatggaggcagtgatgcctttcacccttctggccagagcagcctggttccagagccagccttgaccagagcttgacccatgctgggggaacttagctctggaagctggcagggtgtgggatgagtgactggtgccaggtaggtctggggagatagatagatagtttgataatcccaaatcccaaaactgaaatgctccaaaatctgaaactctttgagcatcaatatgactgtcaaaataaatgctctttggagcatttcagattttggatttgggatgctcaactggtataatgcaaatattccaaaatctgaaaaaaattggaaatctgaaacacttctggtcccaagccttttggataagggacaacctgtatatacagaaagagaactgaaataatgtgtctttttggtgtgtgttattggtgtggaccaatttattagttatcagcgttactattgttgacacccacatctgggcaaggctgtgtaactggcaaactggaccctccaccctctcgggttcacaggcctttcttcctctgcagggagtgggaccttgccggcttgcctggacacagggaccgattgttccccagagtcctggattcccttcctcatcccttcaccgtgactgaagtgctgctcttaatgggcagattgccatcaactgactctgttctttaagtaccggtgacgtcgtcatcttccatgtgctgcccaattacatgattccatctcttggccgttactaggctctcagaactggcctgacccaaacctctttgcactttcttgccaccttccatccctcctgcccctgtgccttgacctctcgctgacccttgttcctgagggctcttgccttcactgtcagaccagcatgagttttgccaaaatacagattcttttcagtggagtggtatataatttgatttttttgtttaaactcctacattttatagtcattggtagttaaattacaaataaatcttacactaaaatttattttattttatttttatttttttgagacagagtctcgctttttcacccaggctagagtgcagtggcatagtcttagctcactgcgacctctccctcccaggttcaagcagtcctcctgtgtcaccctcccgagtagctgagattacaggtgcccgccgccacgccagtctaattttttgagtagagacggggtttcagcatgttgtctaggctggtctcaaactcctgtcttcaagtgatctgtctgccttgacatcccaaagtgctgggattacaggtgtgagccacagcacctggtctttaaaatatttttttaaggaaccttatcatcagtgctcaaagatagtcaaaataaattaaagccatataaaaggaccagtagggcctcaaattggtcatggaacatagggagcaatttgaaattaacaaaataaaactaatgctaactggattgtttttccccgagtaacaaagacatattaggaggatttctgctcctaaattgtcttttaccatcctagagtcaaaatgcttgatccaattcagtaaatggaattgtacaagggattagtgatgctttaccttttatatcagaaaaattctcagaagtacacgtaagtggaatttattcttttattataaaatcagtctatagagactccttcctatgtttacggatgagagagaggaaatgatagatgcttaaaagattaggtaggaagccactgtgattgtccagatgatagcctgggcaggatagtataagcgaaatggaaaaaattcttagagactatagagtcgtcagtgtcactttcatatgggccaagttgataaacttttaataaagctcaggattatttacataaccagtgtaattctctgggataaaggtctgaataagttagagtctttgggaaagcaatcttagagcagaggctaagagcagtaccaagaagtcagactacctgatgttaaagcctagttctatacctaagtatgggaagattatttaacctttctgtgcttagttttctcatctttaaaatagttcagtgaagaatcaacaatatcatgcagttgattatgtgaggattaaatgaaacaatacaaataagacacttagaacacttaaaatatgttatcactattattattgttattatttggagtcacatacacatggttttaaatcactcctccaccatttgctagctgtgtggctgtcagaaaattaaataaactctctaagtacccttctctttattgtgggggttgattgcataggatggctttagggatttgttcagatcaatgtaagtaatcttctaacacgtagactatgctcaataaatggcagcactaattttcattttctaaagagaaaaatacttggtttaaataacctgattgattttaggtagagaataaaatatatgtagaagtgaaatctatctactatatgtcatttcttcaaaattaatttaagatttccttccactggcaaatatatttaaatgtactgactaattaaattcagtttagtattttgtatggatagacaaatagatacaaacaaacaaacaaaaaaaccagacaaatgtatatataaagggttgtctctccaaacccaatattgataaatttttaaatcttgaagatggatcattttgatgaaaacacaggtttgcagaagatactaggttgttatgggtaaatgtctggcttataaaaatggtactggatattcatgcaaaagctcccatctgatattaagttaaacaaatcgaaattaaattcatttcaacaaatagagggccacctgagtgcaaagaaattttcccactcagtgtgtttctgtgtatcttttttaatttgtctaaatctttttaaaacttttaagcagaagcctctgttagggtaatatgatccttcactttaaatctgaagttaaatttccttttatttgctccaactgtacatttctctggtgtcaacaacaattaattctagtaaaatatgatttgatgattcttaggtctgtttttatatcatccaaactttttaaagttgttaaatttttctgaacttattttttccaatgtgtattttcccagcataaattggatccacgttaatagtcttattatgtgctagcaaatccatgcccagtctagagtgcaatactcccagtccgttgagtaactactatgccatttttacaaatgctatagaggaataaagaagaaatgataatgtgtaacctctcatgaaagagatgaaattctggacggagaatttgaaatcaaatgacaatttttgttgcatagttttgttttgtttttgcagttttggtaaacctttgataccatgagtgtttactacgagtgaacaagtcacaagcctcagttaatacaaaaatctcagaaatagtcattattggttgaataattaacctagattatttctgtgagaaatgtggatgcccatgaatgatgttacattgcatatgtcttctagtaaaactgtttctagttcataccaataaaatgaaatataatggtgcttggtgtttctcaaaaggcctcatatttccccataaataacctgggtctaacatacttgacatctgctgttgtagtagtgtacaatatatactcacatataaattggcatggtacaaatcacataagaactaataagaattatttatagacgtgaatggtgacgtgatgtcaaataattctgaagtggtgtgtttgggatcaattctaaaggagaaggaacatgatttgtcagaaaaggaactcagattgcataggaatgaacaagagttgtctaagaaagatgattgaattgggtcagagccaggaacttgaccatttaatgaaacaatgtacagtgaggcaggagagcaggagagaaaaaagaaaaggaatggaaggtctcaagagagttgaaaagggtatctataagaacttactaattgtagcagctgactaaaagtggaacgagcttcctagcaaagttgtgatatccactgatagagataataaagtcaagagcgtctccttataggatgttgtttacggaattcatacattgggtagaagacaaactagatgctttctaagatatgttccacctctgaaatgctgtgattctcctatttgtattgctaattagccaatgaatatagtgtgtgaaagatatgacagcacaattgattgctctgtaattataaattaccccctctgctaattacaatgaagttttttgaagattttttaaatgaagcagtacacttctttatgggaagatcatgtcacattttctaactgattttgtaaatcccatttaaaaaatctcctctactattagaaatgacaaatatcaaaactacaattttatactgagatttatatatatatatatatatacacacacacacacacacacacacatacttttttgggggggataagaagcattaaaataaattaagtagaataattaaaagctgtatgtttagctggtaatttttggagtggcacagattttgttatacactgtggtattcaaaaaagcccagatttgtcccaagtaaataaagaaatgtcttttaaatgttttcacctctaaaagtctggtttggtggttttaatattttcttatgttgctatatcaggatatgtatcttgtgcaaagtgtggcgagaacagtatgtttacatcagtgaagtatgtggtaactagaagagcctaaaaattgtgatttatgttggctgttggcaatttttttttaaaagtacattgactttgtgagaatttttatgtaaataaacaaattctggttttcatttctaatgctatttgattattttgtataaccaaacgggtttttttttttttttttttttgcaccagaaccagaagtagtgtaatgattaatatatcccgcctcatacttattttaaagttgaaattttaggtttttaattcaaagaccccattgtattcttatcctataaaatgaatgggctctgtgcattaaaatttttaccaaatagatatggttttatgaaatgacaaaatgactaaaggatggctcaaaatctctaatggacttttaaaaatttgtgaaatatcagaaatcttgggaatgtatgactttggagaaggataatgcatagtagtgatcagtatcatttattatcagcagaatggcagaacccaataaataattcactttcttaaggacctactgatgcctagtgaatccctgtgtcatcatcagcattatttaaaagatggcacagaaggaaacatctgggagccaattcatcatttaattaaattatcctgtaatgaaatcttctgtaatgagattaattttaattattaatcaggagttgacatgggcacaaggcatcattaataataaatgaaaggctttgtctgttccatttctaagaaaggacctagtttactgcaacggtgctttttatacagccatgctgagtcctcacaatgtggccatccaagaagatggcttgatttttatttcagaacctagagagaggataattgaccaataccttcagatttggattccaaattgttgaagcaatttttgattttaaaaatatttcaaggatgtcaagaatagaaaatgctttttcagagtaatttgggggtaaattcctaattcagggacaaaaatgtcgccttttagaggccaaaaataggtaatgcagtcacattttggtatcagctataggtatgttattgtgctcttgtgtggatggtctgtcagttcctgacctcaccccagatctgccccagtggggtcactgcctccgaccagggttatatgggtaataaggagacggcaaatcagtggacttacttataattcagtgggaactcaacagtttcccggaagtactagctctctgcgtggtcaccaaacagaagccttcctggagactctggccatgtcgcccaattcttgtggcttctcctttcccttagttgcctgaatggaagatggccataaagaggaggcttgagtcttgttctttgaagccaaggaaatcagggttagggccaaagtaacaggaaagcagacagagggtctttaagaacaaaacaaaacataacctgtttgtttcatcacccttaattaaagttcttctttggatttctaattttatctgaactaaactctgaaatctttaacaaggcttctcccactccttcagctttatttcagaacacccctgctgtgcctccttttcccagtttctgtcacccctgtgccttctcctcggcctgcatattcattgtgctatactctctgtgatattattttggcaattggaaagtgccaagacttcttccactttgaggctttctttttttttgagaccagtcttgctctgtcgccagtgttgccaggctggagtgcagtggctttatctcagctaactgctacctccgcctcccgggttcaagctattcccctgcctcagcctcctgagtagctgggactacaggcatgtgccaccacgcccagctaaatttttttttttttttttttttttttgtattttagcagagacggggtttcaccatgttggccaggatggtctcaatctcctgacctcgtgatccgcccgcgttggcctcgcagagtgctgggattacaggcatgaaccaccgcgcccagcctgaggctttcttatttgcagttgtttagtcagttcagtttctgctgctttctcttggatcttttccagatgtgctcctaggcatcctttaggccctagcttagatgtcacttccactgagaagccttcccagattcccagtctaatttcttccagtttgttatattctctcatgacatccattgattctttcaatattgtgctaaattattttagacctaccatgtgccaaggcttttgctttccttcctataacttatcaaaatatataattatatagtcatcctctgtgattattgatttatctctcccactagactgtaagccttgagaaggcaaggatcatgctgaaaccttggcaccatgcctggtacatagtattccttaatgaatacttgataagtgaatgaaaacaacagagatgagaacgtaacaatttaatctgtcagacacttgaaggagcccccttaagaggcaatgcattttccatgcatggaaatgtttaaaaagagactagacaactctgtgttagatttgcctccagagagaggactgacacatgagaaaggaaattgaaatagatgaccataacagtttactctaattctgtgtggtggtggttccaacagctaggaaataatcaatcaatagttgcctattaattgactaagacattatatctacttggtgacagtctctcaatatttgctttcttgaagaaattgaatattttttctaatttattctttaagcatttattcagagtactgtgaaaactgagacaactggttcaatattttagccacagcaatgtctaacaccaaagaatccctgggattctttttttccttttattttataccaattatgttagcaagaacacttaatatattataatagtgatgaatgtatagaaaagggaaaataagtaacatgatcattgtagtgagtaatatctgattaggttgtactgtaaattttgaatgttactactatgtaaagacaaactttttttcagatagctaagctaatcttgccttctgaaaaaaggtttttttagggtaaggaactttactggtgggctctatgtatactaaggaaatctttcttttaattggtaaaattccactgattggtaaaattccactgctctcagctatgtttcaatcttgaccacttagtgtaattaaaaagacagagcagaacaacattgagcctccttatagttctccagcccagtttgccattcagtccgtgaaaatgagtcagtcttggggaggatatttcccacatggcttaataactttaaacttacaagacaacagaaattcgaattagcagatgcatatgctaaaagaaaaacatcttttttaaaaaatccgattttaaagactaaattgtctgtgatgtgcctcaaaggcacatagggacagaattcgtgtaactcctcacattcttgtgcctgagagaagagagtcaaaccactagtgttttggaaccagagatttaaaacagcctagtccagaatggaacttttctttcaatgtgatttgttcatttgcaggttagtggtaatagtaagactgtcacagtaggatttttttgttgttgttcttgagggatatctggaaattgtattagtaattgtgattagcaggctctacctgaatgtattttcctgctaccatgtgtctagttggcatgtgtcaccagggctgaacattttagcacaaactcgaagttgtattgtgtttcagtagttttcctacatgagtctgcatttgccaatctacctatttgatctctcttagcaaggaaaaaaagaaccctagtgacctgcaccaaattctttataaaatagactattgaaaataagcctaatgaaagcttattttattaaggctgtattatgtactctgaagatcttggtaagtattagtataaatattagaaataatacctatataaagaatattttatgatccagttgtctttggttgaaaatttgcttttactgctgaaatttaaaaatcaagccagctacatatagctacaaatagctacatatagctctagctaataaataatactctttagctagttttaggaatgagaagtcatcaaagtgctaattgaccatttgatatagcttttgctatggtacaatgtgtttgttaggtgagagaattcttattccacaaaagaaaggataattatatttagctatggctgatgacctggaagaggcctttttgcatttccttttttcaagatagtttattgatctggatcactaaaagaaaattaaacatctgaaaaacaaaaaactggaagaactgcagtagtggttcagcaatgcacctgtgtatacctagggctttgcttctcctttttggctaattacagaaggactcaacaataagtgctcaagactttgattgctagtttatacatctttatatacactttcattctatctttaaattgtcctaaacataagtgaaaataattagtaacattattcattgggttcatttaatttgttcaacggatatttattgaggatcctactaaatgccaggcactgttcctgatgaacaaatgacagactctgttcttgccctcatgttgcttgcaatcaagttgttaaaagagggataaagcaaataaccatgcaaataagtatacaatccaactgtcttaagtgctgcagagttaggtaagtagttaaatggtttctctgaggaagggattgattgaagactaaaactaataaataggaattaagtaggaagaggttcttggagaggacattctaggtgggggaaataatgtgcacactctgtagcgggagggagcatggtgcattgtgaatggaaccccgaaaacaaagtgaagcatttgggtgcagtaaagctgtgcgggtaagtaggggccaggacatcaagcacttgtaggccacatttagggtcttttttgtctttgtccctagagcaataggaaagctttaacaagttagagagacagtgagtgagcattgcttttagcaaaggtgactttggctgtacttgtagaccataggttagaggcattaataaaggataaaacataatgattgtgatttattataacacaaatggaagaaattaaaattcatgaatccaaaatgatgcttgaggattgggtagggtagaggaaagctctgctttacagaaaaatgccaactattataaatagaaagaatgatggggtgtcaccatttttgcagttcccaataggataactgatcagggtgtcattaacagatgctaaaattgttagaggaatgtttttggggaataatatagtcctgtggtgccaaaatcctgctctaaagactgtttactaataagagaggaaaaaatgtgactttacagtgataatatatcagagtcaccactttaatcaagggattagatttaacttcacaattagtggtacagcctggtatcacagattgtgtgatgtgatagaatatggagtacctaaccttacaaaggaagtgtttctacaaaagaagaaaaaagtcaaacctgaccacaatcaagcctctaaacagaactctgcgtctgtagagaatgtaagaaatggaggatgaattaaattataccataaggtgagacattctataagaatcctagagtggatatttcagacaagattaaagacatgcaacagccaacaaccaaacaaacaattgtattttggttttagaaagctatcaaagaccttcttagtacaattggagatattagaggattttatggtattgttaatttttataggtttgataggagatcatgtatgtttactggtgaaatctgacatatgcaacttatttccaaacgatttaaaagcaatgatacatataaggtacagataagatacatatggaaacatgttaagaactgtggaattttggaggacaatatctaagtgttcattgtactaatctttcaatttttttgcatctatgaaaatttttacaataaaaagttggagagagagagtagatggcaggctgggtagttacttgcttcaccctatgcatctggccgaaaacagtttctggtccttggctaactacagctgttgggtaaatctttcaccaactcagaaagtcaccccaacatctaaaatcccaatctgcgaaattctccaggttctgaatacatgctgtgctgacatcggcagcgtaaatgtgtgttgtgtggtctgagtctcagtttgtggatacctccagcgggaacctggggcttaaagttgaatagtgtctgtagcagagagagtctttgtgcttgatttaaactacacagaatggatggctgtggaacaccagtgaggaggctatgcggtagacctgatagcagaaaatgtgatagcttcataccaggatgctaggatgagagagagaagcacaagatatgagaaatgaataaattttaggtggtaaaaaatgactggacttcatattatattgttaataggggaataagacagagagggatttaatgcatgactcgctggttttttatttgtataaatggaagaatgttggtgtcagacacagaggtcagaagtctgaaagtgggcaagtttgggaagaaagatccaagatttggtattagacatcatgggttttgcaatcaaaatggaaatgtgtcatttgtatatattgggtagttaaacacagcttaaatggaaaaacagaccttcagaaagtactgtagtacattaattttgcacaaagtagcactaatttgatacacatctctaattgctctcacttaaagcagtttcaagacaaataataaactctttttaattgcaacgttgtaaaaaacacttttctatgctgtttattctctaggaaactatgctatataaaaatattatataagctattccttttccagaatttgttcagatatccacaaactgagactcagaccacacacatttacgctgccgatgtcagcacagcatgtattcagaacctggagaatttcgcagattggggttttagatgttggggtgactttctgagttggtgaaagatttacccaacagctgtagttagccaaggaccagaaactctgttttcggccagatgcatagggtgaagcaagtaactacccagcctgccatctactctctctctccaactttttattgtaaaaagaacattcattctttgtaggagttagaaaaagctaaaaacttactttagagcttactttgtgggcttaccatctagtcattagcttaaaaattaccaaaatatttctaccgcaagtaatagatacccattttagaaacattgaaaatttttaatgagtaaatagaaaaaaaggtcactcaatttaatgagtaaatagaaaaaaaggtcactacatcaattggcatttgatgtagatagtttgtactcattttaaacttaaaatctgtttgtgccttacatgtagtgaatgaaataattactttttgtactatgtgtattttactatagttcttcaatgagaggtaactagatggtttatgtcacttcacttgaatgtgtatttatattccaggatggctctgtcaggaagttttctacttatttatagtgataacaattcatacaaattaagccttttatttttacctctaggagttagatatcataatcattcagcctctattaaatctatagtcacagaaaaatgatataaaaccatgaatcctgctacaacactttggatgtgttgtacttttacccactgtcacaaataatttaaaataatttctaaggaaacatcatggcagccccagttggtggtgcttagtgtttaactacagcatgatgactacagaagaggtaacaattttagttcttgctaaaactaaacaatattacatggatgcacatacacataatacaattcagaaaattatcaccaattgcttcttttttttttttgtcttctcagaaatgggcagatggattatgtactaaaaatataaacccttaagaatcaggtttatcctgtaaagaaggtggtgtctctgggcatcacttggctcttttgtaaattgattgtcacttcatttttatcgttatcagtaaaccttattaggtgctgaaatcagtgctcggggtgcagggtcaggtaaggaaagcatgtgctgtgtatcaggagacctggccacaaacttgtcatatgtctctacataagttattattgatgaaatcattgtttggtgagagtttggatagcctgtatagggaactagctaagtagttgggaaaaggaactgaagcaggacaaggtaataaagtgcatcaagatagaccccaggcccatttggttagcatctctgaaggagacaagttcaaataccaagctcaaagagctggtagaatgagtggagctgtatattcaaagtacctaagcagttgctatgaataccagtgtgaggttcactgtttgcctttaaagaccagcaagtgtgtaatggttgcagttaggtcagactgtatggatagaggtagtacagagccattagtggaagaatattaaatttgtttgtttttagaattggggaaatatgataacatgtgtctgtaaagcagaggttgtccctggagaggagaaggaaatatgcaagagaaatggggggaataagttatacttgcttattggaaaaattaggctctgaaaggatgaccatggattcttggctgcctaatgaaacacgacagccagaaagggcctgtggtaagtggtcagggcagaatgctcaatagaattgtgatttttgaggtgataagaagagaaaacattgtatttagagagggactattaagagttctaacttggttgaactcttggttgattaataacaagttaggacaagtccaggtaggtgtttataggtagccaaagtggataaaaactgggaggacgctgactttgaggcagcgaaggactgtgcttacaaggcagaaatgcttagtaacatgatgtcaaatcatcagggacggtggccaaggatggattacagaggtgcataaagtagttgctaaaaacaacaagtaggtttggaagagtgctcctgggcttgatagaatttagcaaccagtaggggtagaggttagtactaatataagtggtgaaaagacttaaaagagaaggggaagttacgtcacactatcagaagtaatcatatccattacaggggttaaaacgaaattgtctctaatgttaagactatatataggcttaatgtgttaaaacatgggcaaaagtcattccatgctttttctgtgtactagagggactgtaggtatagacacagaaaaaccatttaaaagtgaagcaacggatggtataatcctgtgtcctgttctttgccaaattgattttggactatccctctgaatgcaagaatggttcttctgctgcctttgcctcttttcacatctacataaagtagtgcttacatttgtttggagtcaaaaaataataaataaatgaacaaaatatatggcatgtcagatgatgataagtgctacaagaaagacgcagctgaaaaggaggtgagagagaattaaattagggtaggtgagccccattgataaggtggtgtttatacagagacttcatagacatgagaaaaggaagtgtatgtgtatctgggaagtgagcattccagggaaggatgtgcactggccccaaggcagaactgtgcttgagaacagaaaagaagccaagaggcggcagaggagggaaagtgaggagagggactcgaagctgaggtcagagggagtggtgaagtttttgatatcaagccctgtgggccaataaagaccttgccttttgctgtgagtcatatggaaagccactggaggataagatcagaagagtggactgctgatctaacttttcgaaagagtccctctggctgctgtgtgaagaaaggaatcatatccaggctgctcagcctgtgccactgagcagcctgaaattagaaggggtgggaaggagcagttaaagtatggatgtattctgaagatttagccaacacggcttgctaatggactggattttaaatagaagttatttatcatggagcgcaaaacttagaaaatgctaaggtctagcttctatgtttatacttgctatggcatagaccctggacatgaaccctgggtttgaagacccagaacatgcattttaagagaagatcttccaaccatccaaggaagattctgcaaattgaggaactgagtaaacacagcaaacagttgcctaggctacctgagggaatagttcacaggttaaagtagaaagaatagttgctttgatcaaaatgtggcatgagaataaatcctggcttttccacttacctttaaaatttaaattaccatataggttaaattcattcacctgtccatcaaatcatttatccattccactgacatttagtggatacctaccatgttccaaacactttgtaggtacgcaggaataaagcatgaacaaaacagatgaaaataatctgtgaactattctgagagatgtggataaaacattaacagtggagccctttatgaaatccactataacattaaattttaaattccagctggatttagctttatggacttccaaataagaatagcctaataaattctagtttaaaggtgtaacattgaacagaatttagaaaattcttttctaatgttgaaacaaaggacttttgcaatagttgcattaaaaaaaaaacattgactcacaaatgaatagctagtggagggcaggatttctagataagcaggacaaacaatctccactgtttctcaccaactatggagctaatggggacggtggctttaagccaactaccacttaaggggaaaaaaaaacttaccattcaacaagtgaaatagttagtgtggaatggatagcatctttgaaatttgattttcagcagtctccccaagtgactgaatacattttttcaaactatttacttttgtatcatgtcttctcatggtcaaattcaggatgaattttatgacaggatttggtctaatgctgctgaattgtctcataaatatcttgacaagtcatttgaaacatgctgggcctctaaagaggctgactgacatgaaagcactaacttggaatcatcagggcttaagggtaacttatggaccctaaggcagagccccaaatcaagatcataaaattacagacagtgagttgaaattgagaccagtctagattggtaccagcttctttgggtttcttcggtagcagggtggagtcaggtggctggtgccatggaagcattttggatctggtacctactggctacaattcattgttagattcaaggatgtgtccaggctttgggaagcccttctggggactggaaagggagggtccagtacccagaagggcttcccaaagcctgggcacatattcagagtccagccaagtgggctgaatttcaggacaattttctgggtccatccaagggaaggaagcacagattcagatcggaaggtgtagcaagggacaggaaagcctgttttatagtgagctctgagctttggtcattcattaccaggattacctgataacatggtatattgctgccatccagtttaaggtacacactgccatatatatatatatatatatatatatatataattctttatatttctctgggcttaactgaacttagcttaaggcacatgaatttatcattcatacaataattatttagcatttttatatctgggcctggctaggattaacaggtattgacaaagaggatttgagatttctgaaatctttatttagcataacctaaatcaacaactaggattgatagaaattaaaaagcactgatttattttaaaagtgaaaatttctttatattttgatgcaatattttaaaaatatggtacagatttgattcctaggtatttactaatagttctgcattttgaacaccattatctatattaggcatattgaccttcatgatttttgtacaattaatttttgcttaatttattttctaatttatagagacgtgaacagttgccataaccatgaacatttacagatcaatggctattggagttatttaatttatacatggctcagaaggtggcaaccatttctttcaaatgttatatggacagttctagaatgtatgcagatatcctggggaaatggaggggtgggtttctttacagaaaagaatgacctgagatcaacctaacaaaagcttggttgtgaagaaatctatcagaacaaaattgtacatatgaaaagatataattcttgcatgtgactcctcaactgtatttaccaggatgaccctgtgacctttcttttactttagattgattgttgatttatttcttactcatttgttcacacttttactctgtccccaccctgtgccatccttgagctcaatgctttctcttgctccactttggcttggcacagaccttgatctgatgcttgaactcagcttcacctgctatcctggatcactgttgttgtttttcattcctcggtgcttcccgagttccctagcagatagcctccccctagctcaacaataccaccaccaccaccagcacccccgccaccaccccccccacccaccccaccacccttgaccctctccatacacactctggtagactgtttttcggcgtccttaccttgataagcttaacaaagactaataataacaaataacacaccacattactatttacaaagtgtttacatattcattttcctgtttgagatagtggttcctgtttcaaaatagaaaaggaagagtgagatatgggttaaaggaggactatgtttttcataaatatttacacatatttacatatgtaaaagtccacataagtgtttataaatatgtttcagtgataggatggaaggtcaaatatatatggactcttatgatatatacaaagttttatttgtttatcagaaaaatactgctctgcaaatcttctaatttgatagccatttatcaataaattgtattcattattgaggaaatcattactttggtgatgtttgtcatatctgatttaaaagcaacctcttactcttgagaagcgagaacaggagagtgttcattcagtatgagtgaagatggagacagcaacccagagcatttataggagaaaggctggacatgttttaaacactggataaatgcaaggcacggttatatgccaagtcgcattggactggaagagatcgggagacccaagcctttgtctacatgacttgtctgacttatctcacatcttccatttctacatttaaaataggtgaactagatgtaggaccctgttatttcaaatatatgtgacaagagatatctatgcccctttccttttgaaaatatttctggtgtctcttgatatttgaaaaataaaattttacgaatttaagagtccaaaactaaatttcactgttatgagaattttgcttttgcttgtcaccataataacttgttatttattttaatcttatctggttggtcaaaacttcaggtcagctttacgatatatttgatatggtccatttatacttggaactgatccattgtaggtaataaaccaattaaggcagaaaactacccttagacaaagttggtatttttaaatgagctggcatttgaaatgattattttgatgttaatattatatatattttatacattatgcatttagagattatatttatgaaacatttcaatataacattgcatccaaatttgcatatagtatacataacccaaattatggattttgatacatattcttttaatcataccaaagttgcatcttttcatttactcattccatcttcagttgtttataacaatgtgaatggtatgaaattatgtgtttatataataattactccatatatcaatatcatcagaactgagaaacttattagcttatcttctagaaatgccattaatgccttgagattttcttctctctacttaatagggttaaatagatgccttccattttagaaaggaacagtcttattatttcttatttctgagaaaggactagatactccttgagatgtctgtaataaggtaaaattgttaactttcaacatgctgttttcctaactttgtgtatgtatgggacccatgatcttatctagccttttgaaatgaatcactggaattaagaaatttaaaagaaaaaaaatactgtgtaaaactatgctatgaaagaaaaattattaaatatgctcttttcatatagtttgctaattaaatactgcagctgttatgtggagggaaagttgcagatatgatgagatattgagtttgtcatttctgaagagttacatttggctaatctgtcttaacaaactagaattttccatacttttatgaggagtatgcgtagtaagtatgatgtatatatctttgcatagacatatgtacacaaacaagatttactaaaatattttaaattaaaaatgtaaatctaagacctgcatcctcaacctttaatttccaaagaaatataaattttgctgattatcaatctcattcaccttttctttgtaaataaaccagaaaacagcatatttagagagtacattcaacagcattactatttcatcacctgtttcagtaataaagtgacagaatgcacctttaaaataatactttggttgtatcttaaaaattaaaattgagagtaatgataattaaattgttaatctgtctgttaacttagtcaactataagaaaactgattttttagaaaggggtaaagtaataaaaatagtctattcattagattctcccttaattttaaaggaaaagtctcttataatacattttgaggcatattttcctattttcttctgcacactgctatgcagtgcttaagagaaggtaaggacatcttttagagaaactgagtatttctagttcagagttatttgaatgctggttacttggagtggaagacttgatcgtgggcagactagcaattaaagatcatgaaagtctacactccaggaggcgatagacacttgacttgctcactcctttctcagtcaaatgccctccctatatgccatctgtgggctgtgtcaggcttagggatcctgaaggaaaactacttttggagaaataaatgactgcttgatatgattgaagtgattggtttatacgttagtctttttgagttatgtcatctgtatgttccagaagtattaaagtttgctttccttcttttaaattctagatctaaataaatttaattttggtcagatttggggctaaaatttgatatagtcattcagttttttttccccaagggataattacataatctttcttgacgtggacttagaatgtacagtttcacaggcttgaaagactagtagtcagacgaagtcgtattctgatgaacaaaccttcttaagcatgttctgcaatgtccagatagaattaataccaatccaaattcatttttctcagaaagtttattatctttaaaaatcttatcattagcaaccttgggatctcaaaattatagtcttagaacattctacaagtcttttatttgtgcagttccttaattactactatttctacaaaataactgctaatatttattgagtgcctcctgtgtcacttcatactaagtcctcctcgtaatttatttccttctgtcttcctatcactttcatctggcaggtatgctcattttcctgttctcgggcaataatttgccccaggtggcttacctggtgagtagcagaatgaggatttcatgtcctgttgcctggctgtaggctttgagtcttaaccacacaccgtaaggcacccagagtgtgaactttccatatgatgcagaccagctttctgtttctgcaattctgcattttatggaaatggaagcattgttttaaggatgatgtgtgaccaaattacattttaaaaatctttggtaatactgacattgtaaagatgaagatccagtccctgctctgtggagctccaggtttctggagagagagtttatcagataattgcgttgtgttatttcaacggtcaaaaaagaacatggcaattctgtcatggggagggctttggagaagaccagatgcctgagatcaagggagagcaaaatacttcatttttgtgtaacattctcccgtgcaactttgttccacatttcatgatatccttttcatatctaaattcacatggacttctgattgtgctgtgtattccaaaccatgcatttccccacagtttcattgtgtttgctttcatatgcttttccatttctctttactactatgtaaaacatgtattttactttattaggttcttgaggaagggtaattatcctttttaaatttttttttcaaaatatataactgcatttaactcttctttttctgacttctgagatttaaaatgtgttaattatacacttaaaatggagatttacagtggtaatgtacatttaaaatggtgattattcatgcctccctgtgtcctccccttttgttttgtagctgtacttaataggaaatacggaaaagggcaagaggattgcccagatttaacacagtgtctagtaacaattattgctaacccctattatgcattattctatgccaaacactgttgccagcatttttcatggattattttatttactcactttaactttatgcatagataactgttgttatgcctattttagagatgataaaaccaaggcacagagtggttaaataacttgtcccaaaatcatgtacctagttagtggcagagtgaaaacatgaactgaagaagtctgattctagatactgcatgtttcaaacttaattggggcttctcagcttggaggcaaatctgtaatttatgaaccggtcattcataaatattcagtaagcatttcctggctcacattagagaattatttttccgtatgatatggtggtgtttaaatgttataaaggggagcatattggataggtcaaagagtaggtcagaagttaggtgtacgcttattgaaatctgggttctgccataattagttgtgtgactttttaaaaatcagttccttccatccttcaagctctcagcttcttcgtttgggtgatgattaagtatcactccttcaacagtgagtaatgcaaagttttctacccattcttgcttaaacataaattttgaagccttcagacccatgtggtgggcactgttccatctccattacaaatcttcctcctcatctcttcaagtagtttttttttttttttttttttttttttttggagacagagtctcgctctgttgcccaggctggagtgcagtggtgcagtctcagctcactgcaacctccacctcccgggttcaagcagtcctcttgcttcagcctcctgagtagctggcatgcgccaccatgcccggctaatttttttgtattgttagcagagacggggtttcaccatatcggccaggctggtctcaaactcctaaccttgtgaatcacctgcctcggcctcccaaagtgctgggattacaggcatgagccaccgtacccagcctcaagtttttaatgttggatactcctttggtgatctcatccagtctctgatctttaaataccattttttatagggaagattcacacttttatctccaacctagacctcttccctgatctgtagacatctaatccattggcaatcctgttggctctacccttaaaatataaccagaatttaataaatttctacatctttattgctaccaacctagctcagaggttcatctccatctcggtgattgcagtagccttctaacttgtctccttatggtatattctctgcacaacacccaaagtaagccattaaaagattaagtcagagcctgttgttcctctgctcaaacctggcttccaatctcactcaaagtcacaatctttacagtggcctttgaggctctacacaaacaggcccttagttgcacctttgacattgtctgttatctgtctcctcctcagttaattcaccctagccaaaatagcaggatgtggtctttgaatgtgccagacgtatgcacctgcctcagggcttttgcacttactttcctctatgtctaaaacgttcatctcttaagtgtttgtataactcactccctcagccccttcgggtgttttccaaaatatcttttgaataaagctttccctgactacccattttataatagcattcattcctcaactgctggacactccttacttgctttactttgcccaatagtaatgaacattctctaggtagcataataaattttagtttttttgttttgttactcttttctgtctccatcaattagtatataagtgttatgagggtatagattttgtagatttttatttcagtgttgtatacttaaccacctaggacagtccttgacacatattatgcacttaataaacatttgttgaattcatgtatgaataaatgaatgaaagaatggcatttgagttttattaggaacttttatcttcttttgaaaatgatggcaagttagttttggtctcttattctgatagctggggcctatgagtgaatgtatgctgaaacaaaatgcctcatgatccaggccagaaatgtaagtgtatgaaatgctttgagtgcaaataatataggaatgtgggggctgtaataaaccacagggttaacccctaatcagtgggctacatctcattgctatcataggcaaacagtgccttgccatttgacttagaagaagcagctgcaaatccagatttttatgagagctccagcaattcataaatatcgatttccaattcaaaattttgtgtgggtaaaataaaacatcttctagcaagattcaacttgtggactgccaatttgagagctctagtaaaagaagtataaacttcagacatctatctattttagcaaactttacctggtttaaaatgtcgtcagaaaagtttgtaaaataacttgagtagaaaatgtaggacatgaaataccaatcaaatcaagaatgtttgatgaagaataattcctagaatctttttgcagtgtttctggctccctttgccatttctcttgtttgctcttcattctgataagggcctgatctttgatgttgacagtctaattgaaccaaaaaatgaacaaataaaatttattgagttcccattatgtgccatgcagtgttctaggtgccagggttacaagaatgaagacaaaagtacaccttgatatgaaacttccatcctacttggaggggagagaaaggtgacagataatgttcaaattaactcgtgaaaaagataattttacatagacctaagtgcaataagtacataaacaaaatgttctgatagagaatgctggggttaggtgttggctagtttatactaagtggttgatgagggtatgtttgaaatggggacttttgagcacagaccagttggcaagaaaggcaaccgttcaaatatgtgggcagagcttctagacaatggggatatgagatgaagaaaccataaagctgaaaggaacttattgtgcatggagaacagcaaactggacagaggggctggagtgcagtgagggagagagctgaggtgggtgaagttagggaagttggcccctgtggctctattctttgtcgtttgctctctgtatctttttcgtacttcagtttgctacatcttcacatttgctgatcttttccacttcagtgtcaaatgtgctattaatccagtccaaagaatttatttttattttaaatgttctatattttagctttagaaattgcatttcattccatttctatacatttcatttctttttttgttatattcatgtcttcactagattcttgaatatatttatgataatatcaattatcaattcatatcaatttcttcagattatattatctctgttatttctgggcctatttctaatgactgatttttcttctgcttacattttctttgcatgacaagtaacattttaggagatgctggatatttggctgttgactgtctgggttttattgtcttttttatggagtgttaaacctgtttgagaaggctgttaagtaacttccgaatcagcttaaattttagaggcttgtttttaaatggtattatagtgaggctgctgaagcccttaatctagtgttagttttgtcttaccattaaggtgtgaactttcaggatctcagttgaatgtgtccggtatttagtgaggcctctgtactctggctggataaaatacaaatgtcttctagctccctgtaaactgcatattgttaagattacagcttattaattggccctttcttttgcacagtcttgttgtgtttcaacctatgtatatgcagttcagtctttagcaactatatagtgagtccccatgcagatttctggagctctctgtctacttagctgtgttatccccagtaccctgccctgcagacaccagacaccttaaccttccagaactctatctgactgcttaactgaggataccccctcccgcatcacagtccagaaagctactgtagacagacagttgtgataatagtgctgaccttctttgtttcccttctttcctggattacagtcctccactgcctttgtctaacatctaaaatcaaccatttcacttatttctctggatttctaaatgtttcattacaggaagggtgttgggtacaagttctttaacatggtcatgaatgatgttgagaaagattcttttcccataacattttattatggaaattttcaaatgtgtacagaggtagagaaaggaacctaatgaacccttatgcccaccatccatctttaacaattagctacatggtgccaaatttgttcacctataccttcttcccagcctcttacccatctagatcataagaaagaatttagaagaatttaaaatggcaaaacttcatgctgtgttaaagaaaaaaaaaaaaacaggagaagatagtatttattgaatctcttctttatttatttggtacttattcttgaactatagatctcaatctgttttattatcacagaaaccttacaacttaatctttttgaatgctgccaaatctattttaaataaagctttgatttgcatttttatttttgttgcccaaagctttacaaatagtctgatagtttcaacagtttgtataccttatgcatcatcttgtgtatacaatgtgattcatatcacatacaatgtgatttggttgcaactgataaaacacagtaacgtcacaattcattggctaccatagatttgaccagtaatatcttgttcaaaaggaagtttgtgttgactgttttttgttatgaggaggagcttaaaaggtaaacaatgaaacaacatggaaaatgttttaaaacaatataatacttaatattaagtgaaagaaactacacaacactttcatctacatttcataaatggttaagaatgtcatatacgtacatataaaattggtataacaggtaatttgaatcttaaaagaaatctagaaaaaaattaagttagtgatttttctaacagatatgtgagaatatattttttcttttaaaaatttatttgacactatttcatcagtaaatacaagttagaaagcattaaaaagaaaataattggaatctattgttaatggacatatccagctgacttaacttactaatgcatgatgataatgaggccagaattgtagctggattgaaaggagaatgacagaaacacagtcaaagccaagcttaataaaaggaagaatcatgcaagacagaatgaaagttggaaaatcagtcgcctgtagcaaaaccaacaaagttaataccactaacaaagcttcacatgtcttagctcaaaatatcatcagtgacaattgtgaagagttgatcatcttgtcccaacactaatgatatttcaggcatgtatctacaagttcaaatgatagtaagttcatagcttattatacttgaaaacaagcatatattacaataaaatatcactgtgggtagtaatttctttactttggaataaatttcacatgtaaaaaccatgtctgtaagttgtgctgagaatgtttattctcccacaatgaagataactttcctgaacaatgtatcatgatagttattgcattatcctctatgtctacttgacccataagtttctcaattatctattattcaagattctatttttaacatcttgccagagaacagcattggcatgtgagtatgcctgtgcttccatgctgtgtatgtgtatgtgtgttataaaatgatggttcgttagttctgttaggtgacaaatgttgagatgagtgaagactgtccacttttccttccaaattttgcttctaggtaagaatgaaagaaatgtcaaaccaaattctggcttacatatagatattttttgaacatttcttggcaagcaaacacacataaaaagtaactctatatttgttctgtaggatcaaggacactttagctcttatgtccttttagatgttttgaccattacctggtatgaaaactccagttgagataaacgtgaaaattccaagaccggattctctcaccttatgtctagcatcaggcaattagaatcttctgtagttcatgactaggcataggaatacttaaaaagacttagccaaacacatcctcttgaaagtgaaagctgcaggcttatctaatttttacacttgatttacgtaagggtggagagtattgctaaaatattttagtgatctacttatcttgagtagttttaacttctattaaaatgagatgtgcgagttcatttctgccattcacagtgctactcaagaggctactgttttcgtcagtgaaaacccatttctgacattaaatattctaattttcaaaaagttgtttgttgatggaaattcagataaccagaagacttttagtactcatgtgagggaaggtgggctttatgatgagaatgctacctttctgttgtcacatttttgaatagaatgctcatagcacctacagtgtagcttattctaagtaaaccatgcataattactggatgtgctatagtatttttagaaaattgcagacataataactgcattaatgtttgtggtattaagaacaaggttatatatgctctagaaatccttatttacagcttctgtgacaaaatgtcactgaaattcttgagtaagtcttatataactatgaattccagagaggtggaagttgttatctttaactaccaggagtgaacatactaccagtaataatagttactgttattattattattattggacatcatggcagtgatggtgacattgttgaaaagtcatagcaataataattgtcctattagtaataatagaggaagaagataaagactagctactgtttcccgaatgtttactctgtgctatacgtttaaggttcacatgtcatttattcaccagaataactcaggtattgttattttatcttaatttttcggatgaagaatttcacacaactggtatgcagcagaaagggaaaacaaattctttgctctctgattctaaatatgtactaatttccattgccgtatattgcttctttttctgtaaaaccttttataagaatttccgtgaaaattttaaaatacatttttgttaggacttctggaacaagaatattttatagagaaagaaaggaagagttctttagtttattaaaaattgtgccagggaaaaagataaaaatgtggaaaactttatttctgtttatgaaggcattcagcttctgtttaaccaggtccattgaaaacagattgaaatgtagtgttttgttgttgttgttgttttttcacatgaaagtttgtgggaaatcttaaaaacagagtctttctatacaaccttgattttccccagaacttctcatgaagactgctgccacagcttttagacagtagaataataaatctaccttttgggtacatagatatcctagtaaagattcagataaatggttttgaaaaatagtttctttttcctttctttttttctaacttttattttaagttcaggggtacaagtgcagggtttgttacataggtaaacttgtgtcatggaggtttgttgtacagattatttcatcacccatctcttaagcctagtacccattagttatttttcctgatcctctccctcctgccaccctccacccttccaagaggccccagtgcctgtcgttccccactatgtgtttgtgcattctcttccacccagcaatcccattactggatgtatacccaaagaaatataaatcattctgttataaaaacacatgaacaggtagtttattgcagcactattcacaatagcaaagacatggaattagcctaaatgcccatcaataatagactggatcaagaaaatgtggtacctatacaccatagacaactatgcagtcatgaaaaagaacaagataatgtcctttacagggacatggatggagctagaggccactatccttagaaaaatagttctctaatgattgcagtttcctggggatgaaccatactcttaagcttacttgggatggcttgtcctcattgattgctatataagtaccttctataagatctctaaagagatttgtattgttatttatattcaaagattaagcttcttgtgttgaaaaatcatttttatgttatgtatgtcaagaattcaggatcttgaatgtaaaaatcaataaaaataagagggcatgcttttattagaaatataattctcaaaatttcaaaattgttgtattacatagttgcatattgttgtagtcatctcaggctgccgttacaaaataccatagactgggtagcttaggcaatacaaatgcatttctcacaggtctggagattgggaagctcaagatcaaggtgccagcatgatcaatttctggtgagggctttcttcctgtcttgggggttaggggtggggagagagagagaaagagagaaagagagaaagagagagagagagagagagagaaaagagtgtgctcctgcttgctatctggtgcctcttgttataaaggcattaatcccatcgtgatgaccctgctcctcatgacgtcatataagcctaattatctatcaaaggcccatctccaaataatctcacattggaagttagggcttcaacatgtgaactttggggcaatgcagacattcagcccataacgaacatcatcagtttagtatcaccaacattaaagtcaagagttatactacttatttatagtttttcactcaaatggagtaacccatgtgcattattaaaattggtgtattttacttatatgaatacatattttgatgtttctgatgatgatgtagtcatgttagggaacttcgtgcttgactagaattccatggtacttttcctaaatatttttgttttatttctgaggttattatttattatttataactattgttatgaaagttatgcagaagatagaagttgaatgctgctgaagtgatcgtagtgtctactcagttacagatttagaaatgcgagccattattatgaagcaagccttataataagatataaggaccaaagagtatagtgaatgtgaaagagttatgagcataagattgctaaagattagtttactatctattagcatttgaagtgtcaaagcattattcagtgtgtgtgtgtgtgtgtgtgtgtgtgtgtgtgtgtgtgtgtgtgtatatatatattttatttttattttttttgagacggattctcactctgttgcccaggctggaatgcaatggcatgatctcggctcactgcaccctgtgcctcccaggttcaagtgattctttttggctaatctgagtctttttgaactgagattgtcctattttaagagtttattttccattataaaagagcagaaaaggccaggcgcggtggctcacgcctgtaatcccagcactttgggaggccgaggcgggcggatcacgaggtcaggagatcgagaccatcctggctaaaacagtgaaaccccgtctctactaaaaatacaaaaaattagccgggcttcgtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaaaaaaagagcagaaaaaactaactagtctctttatttatgcataaactctaccagttttatttcctagcaaagaaaaggggggacctttgtattattctcacaaaatgtattttgcagaaatatctgaatgttgttgaagtagcaaaatgaactgtagagaggtgaattgttttgatctaattaaattatggtacctaatctaattgtaagataatttttgaactgaagaaattattcagaaaaattccccaaatatccttccattctcacactatacatttcagaatttcacataaatccaaacacttccatttccaggtcaggtcaaaaaatctaaactgtttggtttgtagcagttttcacagacacaggagcccattatccagaaacagctgtcttactaatacttatttctcttaggaggcactccacacaagcagggactcgctggtgttgtgtttgttctgtattaatggttaagggaaggtaaaggggtctttctgatagtttttcctgtggcacagattttgcgctgtggtaatgcttgttgtcattttgggaaaaatagtttttttatatataatacttatgtaatcattttgatacagttctataaaattatatataatatgaaatatatatttgagaaaaataattatctatattatctatatctgtttaggccctgtttatctaaagtattgctaagtcttgctacggtataatattttcctttatggtttgaattaaagcaaatgttaaaggaaaacaaaccctgaagcgaacctcccgatagcttcatagctgctgtttacttactgttaattttattgaagtttatgcttcaacagagcaaaacacagtggtctgaaactagtttaatagaaacccaagtttttttagtgagccctctggacagagctttgtctttaaggtaggggtaatctagtgtattggagggtcctggacttgtatcagaggacttgagtttgaatcttgactgttgagtatcattgttgtgaccttgaggaagttgctaaatatttgaattttcaattattttcctcagtgaaagtgagacaagaatgatctcacaggctcaaatgagaaaattaataagttaattttaaaaactccctttttgttggaaatgcatctataaagtcatacttctagatagaattcacagtgaaaaggaatcatcgaatgtagttgggattttcagatattctaggttttacccatttaaagcaccaaacatttttttctagttgaactattttgattgagcatttttctaaacattattttacatgtatctgctttgtaaaatgcacgggtattattgccttacatttgtatattcattcattaagtctttaacaaatatttgttgagtgctattctgctccaggcactgtgctaagccttgtacataaactgatggaaaaatggacatacagagtgtatgaactagtaggaagacacgcactcaagaggtaaaacaaacacacataaatgatacacacattgtgataactgctaagaagaaaaacaagagggcactatgagaagaaacagaggggaaaaactactgtaggttgagtggtcaggaaagctctctctgagaaagtgaaattgaagctgagatctgaaggatgagagccagccgccttacagaatgtgggggagaggattccaggtactggaaacgaggaaaagaggcctcgtaagaaaaaaaaggccctttttgttttcaagaaactgacagaaattcagtgtgactagaacacaggaaacatggagaagactggtctgacaaaagatacagaatcctgtatagtttataaaacatatttatagcttaaaaccagttgagatgcagaagactttttttgtttccatgtatagacactgagggttaagggggctgacttgcccaaggtcaccctgctgggaggggaggatgaaagctccagcctcctttgacccctagtcctaggctctttgtgctgtgaaaatttcctagctgaacagtttcatacttttctgatgtctgaggccattattatgtaatcagtcactgaaatgaaaccctaatgggctgttgaggtgtgaaaggcccattgccctgtaagatcttctgaattcttatgtctgaatcatagtttttctgtctgctgttgggttgctctcatttgatttcagcaattcccttatctgtttttgaggagctgcagacatagaaaccaacatgtttagtttattaggcacgtgtaaagtaatagccaatggcttctgtgtgagagcccattggattgttgtaatatttgttgtctcagtaggacaggcagtcatgtttgactattcctaattgtaaaattgattttactttgaaaatatttgccaaaaaataaatcatgtttaaatgagggagatatttacataaagttgttgatgtctgaggagccactcttatcccattctcttaagagtttcaaatcaattgtgaataatcacaggagttagacaaagatgacagtaccaggtttattgttaataaagccagactggacagctgtccatgaacctaaggcctaaatcatgcttccccttctttacccaagctgaacttaacaaatgcaatcagacctctgtctccagaaccagaagacagagaaagaacgctatgtacatctggaaggcaggctgcatctgaagaaggtagcccaggaagggttaatgaaacctgccctgtgaccttttcttctgttactcatcagaaaggaaagaagtaatacctgatactcacaagaaagtttttattttataaaatggaataaaaatatgagaaaagagagaaaatggtttcttctacattaaggtaaaagcaaaccattatgatttaattagacaacatttacaagtaaaggtattatgtcttgtgaacatacatacttagtatcaaataggataatttcagtgttacggcttagtaggctaggagtcagagacgacttttacttactttattatttaggagaagccgtagaagaacagtgagctccagtttcgtcatctgtcataacgagtgctataaatacatgctctgtctgagagaaacagatgctactgtgactatgaaagggtggtaggaaaagagaatgacatctgagtacctactgtatgccaaccaatgtgttagatgcttttcaggatatcttgtgttatcaccataatcaaacaagtgttattttcatttttcaaagaaattgagtgtcagattctacaacaaccattttttgagcatttatgataagccagtctctgtgaatacagtggtgaacagggataccacaagaaagtggcatttcaacacaggtatggagagtgaggggagctgggcacatagagggagggttgcctggtcgtgttcctctctaaccagctttcctctacaccttcctcagatggtcttcctggtgtctatttagtcagaaaccgggatggcattctcgatgctcctctttctcttatcccatacacccataggtacaaaggtcctgaaatagcatagagattgggatgtttatggaatagagagaacatcatttcttgtatgctttgagagttagaattgtgtcttaagtgcatagggaagacacagaagcgctttagactggaatattgtgaacctactcttggaagaaggcagttctccatgggtctctcacatttctgtatgtgttatgagtaatacctgatactccagagttcatccagactagattgggtcactggcctacttatcacaatgggtggatctgaagctgactgttagattgtcaaaaacatctccatgtccattgtcgttgtcacagactcccttttcaaggatgtttgtgaacaactttagaagaaagtcatagtacctccctccatagcaaaagcaggcatgcttactgtccagaatgataataaagataatgtctttctttggaaccaagggcaggcttgtttactgcccattataaaaaatatttgatgtccctacattcagggtcgtttgctgcaatacaatcaattgcatgtacaggtgtcatgtcaccttgagggaactggggatcagagaactgtcacaagaaaatgctgatacctgggcttcctttattggaagtaataagtctcattattgtgagtaataagtctctgaccgagaagtcttgtgtatcctgccagcatctgtgaaattgtggcaggctaacttattagcttgtgaataagataaaactcaaaccgttcgtagttgttggcagttttagggatgaggatggggtcctgatagagacataagtttctggaagagaaagaatgaagacctcctgggccaattgatgagatttgagaggaatccatggacatcattagtgaacatgttgatcaacttgtttggtcagttagggaataaatgaccctccattttattatctgttgatgaggaagaattggagagatgattgtaatccaagtacctggaaataggtttaaagtcagctgtttgaaaggtgccctgcatgttactagccctgttccaggcatggagatgtttttgacaatctaacagtcagcttcagatccacccattgtgacaagtaggccagtgacccaatctagtctggaggaaggttctgaccctctttgtaacaacagttataatttcatgctctggcatacaaggaaggaaatttgtaaccactataggtagaaaccaagcaaatagctataaccttgggtggttcccttagaagatgaaggtgtggggtatgtaatacttgttcagaaagtattaaccctcaaggttcttttagttgcaccattttacagcttatcccctcctgtagactatcacaccaaccttagattcactaggagacttagaatttcttagagtggggccaggctgctataagggagctttggttaccttgggggagatttccttgaaagagaaaagctcccataggaagccatggatgaggtattgactagtctttgggccgttattgtcctagaatgggtttgtaatgctgatgaagattacctattggaaaatgagaaactgacctaatgaaatctagacaaattcttaccgtcacccctgttgcctcgcaaaactctcataattcttttggtgaagcagtgatagaacatgagatgttataataatggggcagtaaatgccctcaattgaaacagactccttaaaaaaaatagctgctgcgcaggtcaagcccaaatcagacaaatggaacaaattctatagccacagccagaaagtggttatggctgctaagtctaggggttcccagaactggaatagatggtgtctgtaatgatcttgctgccaaatatagagcattgcatggaatgaaaatagtagaagtttatttgattgagccctgagccagctgccaatcaaaactgaaagtaaataaatcttgtttactaacatagaactgttctctccctctctgcccctgattccccctcctcggactctacttcagctttagcttctttaaggaggaaagaatggagctgggttctctctgtcctgaaagaggtccacagttcttacatgcctctctccaagtatgctgggaaactctggcgagggcagggacttaaacttttatggctctgtgagatacacattaaggaaatattaactggcccaatttatcatcctgcccagtccgatgaaggaggggatagctgaattcagccaaggtggtttgaacatggtttgcagtgggggaaggaagctagcagaccttacgggtggggcctttgttccaattagtgtgtctttgttgtagcttcccctgctgaattgtagatgactgtactatggatgtttatgaagtccagaggggagtctcctgaccaggaagagttgcaagtacagaagtgcttcttcctttggggccccacaaccctaatgacctctttggagcttcagttctctatgactgatggtattgctgattggagcctcttgcaaaaggaagcagcattcacccttggagttctggactcacagctctgacgttgaaacaggtatattcctttaaagaagggtctcttctagttaaactgaatgcctggcccgtggagaccttgtaactcttttttttttttttttgacggaatcttgctctgtcacccagtctggagtgcagtggcgcgatctaggctcactgcaggctccgcctcccgggttcacgccattctcctgcctcagcctccccagcagctgggactacaggcgcacgctgccacgcctggctattttttttgtatttttagtagagacagggtttcactgtgttagccaggatggtctggatcttctgacctcatgatccgcccgccttggcctcccaaagtgctgggattacaggcgtgacccaccgcgcccggcgagaccttgtaactcttgagcctgatgttcccgctttcgggtccagtcatactcattgaataacaagggaagggcccaacaggcctgggttgtcaaatggaaatggcaaatttaggaatgcagccagcctgaggcaagcagcatcttaacctcacttgagaaagtggcagctctaatctcccactctgctgcctgattcaccaaggttcccctaagtacctgggcctgcctcacttatggatcagctaacctgaaacctaatggtatccagtgggttatgttcagcctcaccatcacctgctcaactagaaatggacattgtttctgtgctcagtgttcacaatcaaggaagtgctcttagccctggccaatactccccttaatgaatcttgctgtgtttttactgtctctgggagtgttgcaagtgttgccaatgacctaggtgtttgggctgccacttgaaaaaatgccaactggaagattataaactcccctctttggggccacaagctgtggaaatggttgggctgtcacagttatttatatagatggtaagggcctattctctgatgaaaagcaatgggaatcaagctgctggttccacttgcaccacccagattgccaccactgctgcctcgcttcatcagggtgttggacatgcgacacacccacagcacagactgggcacaaggtgaaggacttctgatatcgaggtgactggcatcacagacttggggctcctgccaatagttgacctgtttgtctcaatgaaggaagacacattgtatgtacagggagttacttctgctaattcttaacagatttactgcatttgactcctcttgaaactggcggtggtgcctcactgctgttgacacattttcaggttatgagtttgccaggtgcattccagttagtcaaagccagtggtgcaccttttattgtagaagccagggctgatagtcaaagtatttgatagtctttccatgctccctacagtacataggcactttgtattgttgaatgttggaatggcttcctaaaatcaactcaaaaagatttaaaactctacctgcctaatctcctcctggtttggacacgttaataagacagttgggtcattaactgcaactggcccagttagggatcatcctctttttgtcctttcttggttatgatcaggatgaaaataggttatctagaactattttgaaaattcaggatatcatcctgaccatttctagacatgatgaatctttctttctcctaatacctatctcaggctagactgtttggtacatctgctgggtggcagcctggtcaaaagatggaacaggaattcaaatttattctggttcagcctcttgagttttcttattggattgcattatcttaggatattgatcgcttggttgggtaaactgctcaccaagcgcccccaaaccttgtatccttgagtcttcttcctgtattcacgaaacggaggctagctaccatgttagcttgcacatagggtaaaatctcagacctttcataattcttgacatctacttatgtattgaaatagttttagccacttcctgccgaatggattgaagagttaatggtacaaattgggagtttatcagttgtaccaggggattttaatttatggaggtgactgagtgacaacatggagaggccaatgccatcgaaagattatttttattacttacatttcccaagaggagggggggcacttacattaaatgtaagtaattttattacttacatttcccacgatggggcgcatggggaagcactgggtttggtcaggatgcagaaggaggaagggaaagcctgggccatatcctttagtggggtttctgtgggaaagggaagggcagaggaaacagtttaggactggctagtttgagtaatttcagtgggctgtgggttgtaggggtgatacctggttgtctggtacctggccctgggagatttagggaaggggagtattgacttactgatgagaagttgataaaggggatagttgagggtgtggtctctggattggttagctgcatatgaagggcatgatcccagaccagccctttgcactaagaattggctagctctgggagtatagtctcttcccattcagtgaggccacaaattccagagcatcggaatacagaaaataatgaaatagagttgatataactggccttgtgataaatggatgccaaatagacaaacacaatataagaaaacagttttagaggagagaaactgcttaggggattactgtagtaatccaaataggagatgacattggttttaactagagaagtggctgaagagatgaagagaagtgaacagatttgagacatattttgaagcctgcatcaattcaattgatgatgaatttagatatagagtttgtggaaagggaggactcagtgactcctgtttccggctttacctaaatgaaacattttggacggtaaatatagcattggtagagaaataataaagagttcagcactggacatgttcaagttccatatggctgtgagattatttatgtagaggtataagttaggcaggagagtgagagagtttagaactaagaggatgaatctcaggtggtgataaaaatttgggaacctacactatatagacagtagttaaggcatggcactagatgaattatccagtagagtttgaatgactgggggcagatcatgcatttgcccacagccccacctccatttgacctgcttctctcttttaaataaccatcttggcttctgaatgtatgtgagtttgtcagccttaataggcaaagaaggaagacagttaagcaaatggcaaatgaaatgattgcattgatttctacgtggtatttgttatacagtggcatggacatgatttgttaagtgtttaactaaaagtagtcccttaggttttggattttatattctatgtagataacacatcagtagcctggctaaggaagctgtggtggctgtttagttttgatttcatgtggtctttttcaaagtagcaatttaggggcttattgaacatacagaaattgtacattgtttttcttcgttctctaatgcttgccagtagttatcagaattacatgactatgcttttgtttttttctttgttctcccatgttggctctgttttatctcttaatgtcaaatgtcatttcttggaatcattcaggaagttcttttagttagtttgccagtaatgaaaagtggcaagaaatgctgtaaaagaacctggaggtatttcacagctttggagatgaagggtgcttgatagattactagacaagtagtcttactaaactgctcaccaagtgcccccataacctggatccttgggtcttcttcctatatccgtgcaactgaggctactgtgttagcttacaagcagggtaaaatctcagactttttgtgattcttgctgcctacttatgtgtactcatgctgagaagagtagaagacacgaagaaaggaagtatgtaagcagcttccttgtgctaagagttatggagagggagtagacaaagtaaatgtaaaggatgcaccccatgaaactgcagtaattaatttattttcttgattttaaaaatacgtatatatgtggcaagacttctaccaaacttaacaggtgtcattactgactaaatcttgattcgcatattactttgaaggatattcatgcatccaacaaataccagtgtagtactgacaatgggaaagtcactgctatttaatatatcagctgtcaatagttcataatagcttcacttttatcagaatgtgtggctgactctcatagattgatggactcaaaagcactgtatagaggttttggaggtcagggtgccattttcaacattttaaaacaagggaaattaagaatttacccatagtaaggctgtactggtttttaagaatatccagttcctttgattttggctatcttattaactattaataattcgtacctacaggatgtcataataattagcagcattgaatgattgctatgtgcttggttttaaacatggggtaatgaaaaccgttattgttaaataaaatatatatggaagatggagtctttcagaatgtgtggttcatattcaaaaataaatactggcactctgagatggaaacctctatgtttgttcccaggtcacataattaattggtggcagtatgctggaaccagaagggcatcctagacatctctgtttataaaatgttattaaaaatcatctatgcaattttcccacttggttataatggtacaaagtaaagggataccatttctctgtttctaggaacactttggtgtcctggtgtaagctgagataaagattatatttctcatcagaaaaattatagttatttgtactctttaaaaattgaaactgcaaatgacatttgttttcacgtttatatttataaagtcttcaccacactttgtttgttgttcctacctcgccccgctttaagattagggaagaaaattacaatccacccaggcttctttaatgcatgaagaacttattctcccagagttttaggcaaggttcactgggcctctattgtaacaggaacagacatatattttgcttcagtgagtgtttcttgagctcataagcattttgaaaatattgaatgacttacagtcccttaagaaagtagccatttaaaaaagcatattcagttgaactttaaaaatataatttataccacaatactcaacaggctccaactgcttgaaagtatttggctgcaaagacattatttttagtgatcctaaccatactaggaacaaagaattgcgtctgtcaaaatgggatctctggcgctctaaaataatccacatgctttgtgaactttgatgaagtcaggcagctctctgtcatataaatatcatagtggcatcaaaacctgggctgaccatgtgctgcaggtggaatgcacacactcacccagtttcgcagcagaagcagctgacactagtagggagacagcgccgtcttcagttcctaaagccattggggcgtctgtgtgctgtccaaacctgaaataggtaaatgattcttcattcaatcctcccttgaaaatgaaaatgacttcagtaaatacttgtgctacaaattactcagatgcagtatttactcttttcctgtacgtcactgtatcatttagagccaaactgtaaagattatgagaaaaaaagaaataaactcagtgttagataaacaaattagagacaggaaatgctacactgaaatactgtctgaagaacttatgaaatgagtttaatctttcttttaagatctgttaagacatttttgaagggatgagagatgagttggaagatactgttttctttaaagaaaataattatttttcataagctattagaataaaagggaatgttaatatcagataatagcattattatctgattttattatctaatcattaagattactcacattatccttatctcttgaatttatctattcttgtttcattttcatttcattagaaattaatatatccctctctttcgtaaacataaaagactttattttataaaatatattacataaaatgttaacatctgtgtgagcatttataattgtgttcatcccttcagtctacctcagatgaaaatatatacaaaataaattaaataaaaatgtctgtaattatctttccattttgttgacatacatgtgtacctaaaactagaagagagccctgtactttttagagtgaagtaataatgaaagtgaaatcttacataccttgtcttatccaaaaaaacgagaacaaacaaaaatgtaaaatgttggtagggaactttaaatgaataccatccttattgatattaacttgccataaagtgtttctcctacagaaatgttttgctttaaagaaatttaaagtaaaatttgctttttgaaaatttaatgcagggatttaattctgagtggtttacaacaatgggttatgaatgtatcaccatgacagcaggttgctagagtctaggagtcaggagatgtggcttcattaaaagcacctccagataatgccctacgttagatttcactgaaagacaggagctgctgatgagggtgacctctgacatcaagtcatcttactataaacagaagggcgtggaaagccattggtcggaaccaggagtgtttactatccaggtctcctcctatttaacctacataactgcactgctcctggagcccgaaggttcacgccagagcaaggtgcttttaaggctacctgggtgttggttagagactataaacctctcagagcattctgtaaacctcgacacagtttcattctactgacaatggagactggaatttattggctgctgaacttcacacacacgcactacagaactgacttccaaatacagctggaataaatttcaggttttggggttttgaatgaggtaagttgcatttagcttttggttgaagcgttatcattattattatgttccatgggcagtcacacatttaagtgtaggcagttctatctcagagaaaaatatttttcttgttaaaataggttgtttagatttttcacagttttttagtacaaggatgatagaagaaacttgtacataggaactgagcaagccaatcaattagagaaaatccctgatctgtttatggctaaagtgtgaaacacacttcgtctttgtttactttataaaattcagctattcaaattacattatgttaatgttgggagaaatgcaggtaacgtgataagtgataatagattacatttcactacaaaacttttaacagtcaactttgttttcccctaatagtacatgctactgcaggattttaaaaaactaataccaactatttaaatgaaaggcttttttctgcataatttgttttgaaatttgtgctgaatttgaacttttcattagatttgagatgtagtcatcttgatagatatgtcatatttttaagagaatgagtgtttccctccatttatggaactattttttcctgaaaccataatagtaagatcaagtttaattttaactttttaaagaatcctgtagtgtttagtgaggtttaccttcctttagatgtgtactgagcacatactttgatttaaaaccttgatgtggaaggctttggtatgctttggtttaaattatttaaatatatctttgtgtctttctcaagaagaagaagtgtaaattgctgttttgctgcatcagagggttgagatgtgggagaattgtcaaaataataacagcatttgttttgttaaagacgactgagggaagctttgctgtgttgggactattttattcttgtgagaacaacacaattcaggcggagaaaatagatctattctaagtatttgcgagtactgatttactgttagaggtggatagttagggaagctgctggaagaaagggaggatagattatcccagccaataagttttcccgtttggtttacggggtctactgagcatcagtcagcactatccagtttcccacttcttttctgagttgtaggtactattttctgaaatactctgcaattgaataatgatgttacactaccttattttgcgttttgtttgttttctaaccgaaagttcactaaaccactaaaatgttacttatcaaaaatgaccaagaaaagcaaataccagtaaccatataatgggttctaactttttgcagggggaagaagagggacagagaggccaattttttatctctactttggtttttctcttttcttctccaagttccttataaattactctcgttagcagtttcaaaattgtatgatctaactttttgatcctgtagcaaccctgtagagggagatggttgtgtgagtccaggggtggcacacattttgctttagggaaatggtgcaagccaaatttaacaggtaatgcagctgttgaagaaatagaaaagccagatgacttcctacgtgtattgaggaaaccaggataaattgtccttagaaccagctcccagaaagccattttaattgaggtcaaagaggaagaaattgtgagtatatctcaattatttattaggtttacctagaatgagaccttcactacagatatacctaatctataattcctatactctttaacccttgagcattcctgctgtatgaatccctttaacatttagactgcagagtgtatttaattgtttatgacagtccagaatttagggacataaaaattgtgaaattaactgactaaattttactttcatctaaggaataagcttctgcatataaaaattattgtaataaatttcttagattttagttgaagttacacagttaatgatgctgtttcttctggtcacatgcttgtatgtgtgaaatgtttcacatagaactattttcagctcttagtttacaggtaatttaaattgttttttctttaatttgacaatttgagtatttctactcaaacaaatcatataatttgcattcttggtgtgtaaattatgctacagtcagtgtcttattaactactaatgaagaattttattttaggattaaaacctgagaaaaggtgatttgtaatctttttttataacaatggttatagaagagagatttaatataaaatataaaactactttaaaatagattttaaaactcaaaaatgttgctacgatgttgtggaaaatatcagattcaatccagcatgctataaatacatttttttttattgtataaagcttaattttaaaaataaagaacttgatgccaacattcttgaattttctctttataaatgttgctattttaatggaagatcaagaagataaaatctgtaaaattcaaaaatatgcctgttttacataattattttatcaatgtattgttctagaaatatttatattttatataaattaatctacagatatatagagatatatgttgttaaatttaaaattgctttaaaattttgtgtgcattctatcaatttgtttttaaagttaaagaatttctaatttttttaaaagttatggtgatactattgggcacattaggagagtttttatatatacttttaaaatgttttttaaagtttaggcatatatatgtacttatacagacatacgcacacagataaactttaacaaaacttttgcatatgaattgaacaattttatattggtaagtgagacaacctttatagtcaattattgttcaaattgaaaaggttagttgtcatttttacttaataggtaaatgatggtgtactagagtatattacataatgattgatattttatgtttggaaacatatgtaactatgcctgtggccaaattcttttgaagtttaatgaccaagatgagctgcatttgttaagtggtagagcactggcttgtctggggtccttttgtgcttggcagatcttgataaatgggtgagtttttcttaaccttgtggccaaaacattccaatatctttaaaaaaatttataaagatatagaatatgttgcgaaaaacatgagcaaattttctctatttaggggactaggttaaatttttaagatgatttttaaagcaatatttgtgagaattcataaatattcattttaatgcagggaaatctaaagtttctgatatgtaatactgctggaattcttctttcctggttcactgcgaagctgcacagtagtcagcaattttaatttcgctgcagaagattctaccaaattaacattatgatcattaagtgataattctttagaatattcattttgctctttttgctatgctaatacattgtaaagcatttatccatctttcttgtgacacccaggacggcaaagaaacatataaacaaaaggtataaagaagaccaagatatatgaggaagtatctctttaggtgtttgtttatcatgcacatttgtgaggaaaaaaattcttttagcgcttatgtatagtatttatgcatagtatcaaggctggacaatctagtcttttttccaatcttagaatgggagctttcatatgagctgtaacatgatatgttaaagcatattatggttccttttgtggcattttttaaaaagtaatgtgttacctttgcctgccccactgggtaataatgaaaaaatataccattagaaggagtttgaaattttcctaaataattcataaagccgatatggcatttatggcaggtgaaaaaggactgtggcatagataaggagaaaacgtgctttggcattttaaaggttaatgtgatttgacatttggtttaatacttacatactgtacataaataattgtgctttgtttttctgttattgttgctaagttgctttttgaatatattcaaccttgctatgaactctcaaatcacttgaaagctattgtgacagttgtagttgatgaagagttatagactatagaagtttttccacttaaaacataattcctccctagtgcatgattttgtctaacctccctcccccaaaccagggtcctggaaggccctcgcattaaaggccagaaatcaagtgagatcttgcctgtgacagcccaatactaaaggagtaaaataattttgccttcaggtttttggtggtggattttatttattaattatctaactccactattcttaataggtatgttattttgtctactgttgcacttagagtatttgatagtatctttgatgtgtagcagttaaagaagggttatggcaggttttctttgaatggaagtagatgcagacttccaggtatgcagtagagtcctctcactttcatagtcaataaatggtgatttgggcttttagaaacacggttttggttttgccagggagaagcttggttaaggaatgataaagaggttgaaaagtgatatagtaagaaaatggaggaataaatagacttccctaactacatgaaaagaatttcagaaatgtatacattttacagctgcaagatctttgtttctaaggaaaacaaagacactttattaaaagttttgtattatttaataattggatttttagaaactgtatttctaagactttaattttttgaccaccatttggcaaaatgccattttattttccttgaaaagcctctcaaatttgtataaacatattaaaacagagatatattttttaaaactcttggtgacaacttttgtgttgcaaagatttaatgtttcgagtttttaaaagttaagaagtgaagtataacgtaagtgaaagatagaaaatgagtcgtattgagaagtgttgacacatttttagccagttgagaaagtgcatctccaagtttatatccctatgggcctagcagtacaccaatcctatgaagatcttcacaacataaatcatatttacttggatccaaaaataatcataggttatcacagtaaagtgtacatcttatgggaagtccttgagtggtattaacttttaaatattatatttaggatatgagtatatattttctttcacacctgtacgtaagtaaactttggaataatttttagtaaaagcttatattgtaaaaattgtttatgttttgcctatttcatttattacattaggactcaatttttaagttaatacctacttttacatttttttatgttccttcatacattcctgaggtatgtaataggaaaggacatatgtaataggaaaggatatattttaattcatatgtattaaatgctgctaactagaaaccaggataatctagggcagcccattaaaccctgtatcagtcggttatcaatttttaaaaaaattaatgtgataagggccagtcaattgagatgcattaaataagaacatgacagctattcctggaataaagctggaataattatttaaaatacttttaaattacattatttaatttaaaattcaaatatggataacttgtaagtattagaaaatatgaaaaatgtcacatagccatggacatttcaagaagtcttttattttcgctaaattagaagccaaaggtaactaaagttagataccattctgcttaatcaggcaataatttacaacaattttattcaacccagtcataaaattcatttcataaagtatgcattcatataagactctaggcattgatatactacagttttttgaaataaatatcacttattcagtcagattcacatcttagagtattcacattagaaatgccaacaaagccgacaagtctaaattgttgttcacagtaattctgtatcgttttctatgtttacaaagagtttggcatgcaataatctgtaatataatctccatttttggaaaaaaatatttttctatctgcttatccatgttttccttaagcaatagtgcatttaatctcatttacattatattttatattttctacacagttcagtgaagaagtagtcttaccatccataagaatatatattaataaattatttattacataaactttcatttgtgaaattttaatctcgaatttatagttgctgaatccaccttttgttttcagtgtatagtatgtcctaagactcaattattaatgctcatttcaaaacaaaatgaagttccaaaagagatcttttgaaaatgtcttcttacattattctattctttacattttttattcccattaatgtttcacgatttgtaatcagaactattcactagaagataattgggataattgagcccagctattttggaagatgctagatataataaaagaaccttgctcttatttgcaaactttttttatgagttaattctcctcaaaagattgtgagcttctcaaagcacctagtggtttggaaacattaaaaaaattaagattgatttttatatatgaatgtaatagtcataattccaaagagtccagaaattatggtattttctacatatattttccagatattgaaagtattaattattctcaataccaaatcacacattctttattttaaagtatactttatgttacttacgtaaatacaacctaaaaagcttgaggggtgaatagtattgtgaaataagagatgcatagtgttgtgtaataagagaaatgacatttaaaaataattgtgtgactaatgtaataatattgaccagtcctctctactcctttattctgctttattttcatctcagcacttaaccactgctggcattgtattgttcatttaccagtttacttatttcttgtatgtctcctccactagaggaggaggtgtttgttgggcagacaggtgtttgttggtctaggtgagtgctgttcagtagaaaaatgatacaaatcctatgcgtattttaaattttctagtagtcacacttgaaaagtaagaatcaacaggagaaattattttatttaacacagtatacccaaaatatcattttactctcaatatcaaaaatactaagatattctacttcctctttttcatagtaagactttgaaatctggtgtgctttttacactaacagcacatctccgttcaattcagccacatttcaagctctaaatagccacgtgtggttagtggctcctgtgtcaaactgcttacgtctagttcatctctgtggctggaacactgcctcggatatctcaggcattcagtaaagccttgctgaatgagcaaatcttgatattttggacagaaaaagaaacaacgcagaagttgtttttcacattagtttttttttttttttttttttggctgtttttggggtggtggcggaggtggatgctagagatatctggataattttttcaaaactactatttattttgcttacatggagtttaaatgttactagtgcttggtaaaaactggttacttcagtagtctagtttaaaggattcctttaatgactgatgtattataaaagtaactagtaggttcttctttgaaaattgttccacagaactttaatttccactcttagttttaaatgggagatattagtcaagggtacaaagttttacttatgcaaggtgggtcgttcccagaaatccactgtacagaacagtgcctatagcaacaatataggcactaatattgtatactttaaaattcactaagagagtagatttatattatgttcacacacacacacacacacaatttgggaggaaatttttggaagtgatggatatgttcacagcatagattacagtggtagtttcacaggtgcattcttatctcaaactcatcaagttgtatacattaaataggtgtacctttttgtatatcaatcatacctcaataaagtgagattttaaaaagaaaggaaagtaaactaatgaacacaactggcaaatgtagcataggagagaatgaaaggcttgatgtgtggccttaagttgtaattccagccttgctagtgttagctgtgggatattaggcaagttgttgaacctctttgcacattagttttctagttcataagaggaggattttattaatcatacctgtctacatgcattaaatgagattatatatatacatagtctagcagagtgagtggaaaagaatatttgcttaataaatggcagtaattattattgttatgtaattgaagtcaaattttaaatattaagacgttttgatcttcattttgaacatttttaattcatatgtttatgaattttaggttcttcataggttctggttaaaatgccttcatagatctggagttgaaatttgctacataacttgctttcaggttgtttctgcttctcagtgttactatatgggaactgggaagaaaattgtatatatctaagcactgtgcctggcatgtggaaaacactcaaaaattttagctgttattcacccattccttcatctcaaaaatttagaattttgatatttatgttttcattgaatactaatagttgcttttcttaaaatagttaaatatttaactctttttcatggtgcttggaattttcctgtatttttataatttctctgaagagatattgcataggttaaagttcactttatttgcagcaagttgcattcatttacgctattcatcctttctgtcttggtgtgtgtgtgtgtgtgtgtgtgtgtgtgtgtgtgtatttctatggaaaaattttcaacatttatttttttatttattggcccacccccttgtgtgtcttgtgaaaactaggatttgaatttaactttttggcatgaaattctgactcatttcttgacctgcttgttttccacttcattcaaaacacattcagaagtaagcctggggtgatggtgcatacctgtagacccagttacgtggaaggctgagacaggaggatggcttacactagggagtttgagactgtagtgcaccatgattgcacctatgaatagccactgcactcctgcctaaatgatagtcattgaaggttctatatgttttataagaatattctttcttatatagcatatatgggtcagcacttgccatttaatgaagtctcaaatggaagagtgtgttcaaattaggtaaaactatccatttgcctctacttctttgatcaaaaccctagaaatatttttcttgatgattcctttaactcctctaccttaattcagaaataagatagggttttgaagtaatttgaagatgtcataaaataaccttttttgtcttcggtatatctaatctttcttttcaaatacagataaaaagatctgataatattctagttaacacttcaatcaattacaatattctgtcaattctatctcagaagtgtttgtcaaaaacaccctctcagttcaccccatggctcctgtcttgagtcttcaccacctctcactgacagattgaatggttcaaagtagcatttggggaacacctactcagggctggggatataatgatgtaccagatcaatgccatctctgccttcatggaacttctactatccgttttgttgcattaggcttttcccaactttcagccctcacctcatcccccttccacattaccttctatagcatctctacccagtttgtttttttacaacctttcaatggtcttcactgaccaaaggttaaattccaaattctttaccgtatataagggcttcataatatgcttcctctggcaaacttcttgacactgaaatacctactatttcccacatacagtctctgaagactcaattcagaacttactttcttcattgccttctccatcttcctttggctaagttaatttgttctatgaacactcacaatactttagatagacttcccctagagtagtcatggtatcctaaatattattcttctctcatttgaccaagaacaaatgaatgaaacctctaaaaataaccactctgctaaatggttattttaatatctaaaggaatttctaattcattctttcatcacatgcctgttaagcattggttataagtgtgggactctgtgggtactaaatagcaaaaccctgtagatgtgttctctgcctttactgttactactttgagaataaattaaaacaagcagaactttctagtaagttctttggggtaattatgctttggaaagccgtgggtgttaaagactaaaaaaagttaaatgataggaagcttgtctgtctctgactttcagttgcgagcatatgtctagtttgtggtggtctggagcttcctcagccagtcccaaggtccagtcatcatggtcttgaggaaagtgacattcacctggggataatttctgtgtattcattgggcagttcatatagttttttggtgataaatcataggcaagaaccagtaggaaagaaggttctttttttttcagatatgctttccttttaattcttgttttcctcatacaaatttaatggacataagacttgtgggatttggagttaattttatgtttacatttttaaaaaagtatttctccctccccaccctttccccttttctcctcctacgccttattcctcttcctcctcctccttgttcctcttcctcctcctcctcctcttcttcttcttccctcccctcccctcccgttacttccttccttcctttttcctcctcctttcttcctcttctccctctcctcctcctcctcttctttctctttttccttttcttcctcttcttttcctcatcccctcctccttctctacctcctcctcctttttcttctcttctctctgtgaaaccgttttttttttcttttttttttttttttttgcccctccatctcatggtactgattaccatgttaaaggcatagaagacattacataataaaacaggatgacagtctctttgctggtcagttttattttcatattgtgtaagtgtatacacaagatgcaaaagagactacagagttaatcaagaaaataattgatttcccagattataaaaatgtctagactttgtattaaaagcaaaagacgggatttttgaaaatgtagggtttttgtgagtttgtttgttaaggatttttctgaaacagaggaggaaaaaattactttgtgagttggaaagacaatgctgtatcttcaaagcttaagacttacttttaagtggtactgttcatattgcaattcacattgcacacatgttcctcatggtatatattcagtagaatttttttttgcttccaaaccacatattggaagtaaaactccttggattccataggttgcatagttaagttattgaaatatgaacacccagaaatagtgggcttataaatgtatattgtacatttttcatattaaccaagagatttttgaagtttagtctactctgcatatacttttaagaccataataatcagatatagatataagtgcttgaagtgcaattgtgaaaatctatggatttcaccatctttagaggatgtaccacatacttttgtccagaaatagatagcatggcctagaagtatgagttattctcatcctttacctctgtgggttaaaaaccacaaagttgaaatatagtctgcaagatatactgaaaatatatgtgtgaagcagtgctaaacttaattgatatgttgagacatatataaaacatacagccactagactgagaaaatgaaaattagtgtatgaacattttagttaatgctgtgctttgcatcaaaacctggtgtttattattatgtgcttttagtaggagacagaagcagtatcagttggaaaaacatttcttagtggattgagaacagtatgctttctagttcctaagtgggaaacctggttctttggtttctttgttcctggttaggcttatccccagttatttcagctgaacattttacatactaatggagtccagggaagtgtatttggcagctgttcatgggataataatgaaatacactatttagtactgtatgctgctgctgtaaagtgtggggggtattttatagagaacttggtgaacagagctatatccgctttaatagtgtgtgaacctgagtgaggaagttgtattttcatatgagaataactagtactcttttcttacaggtaaagaaaacgtgcacagaatcagatgtttcacaatctcagaattccaggtacagtaaaataaagaccaagactcccctaaggagaattgcagtttcggagcactagtaagatgttatacctgagacacaattgtgatttgaaacattgagcttcttgaagaacttatcttgatggaaactgtatatcatgctggctagcgtaccactactcataatattagtcaaagggactgtgattgaagtcagttttaaggaacaatagtggaagtatctctagaaatacgtgaaaaaaaatcccttggcattaagttaatgttcatttttttgttctaatttctttttataatgttttgaatctaaaattgtgccagatactgaataactgtcaaatctgtgtttacaaaaaaaaaaaaaaaactatgatatttctttttctttgcagtgaaacattgctagattttgagaatgttgtcaaattaggaaactcatagttaatttggcattctcgtttgggttgtttggagcctgtggaaatgatgaattaaagtggtgaggccacttcatataggaatttagcaccatttagttgtttgtggttgaaccagatgcccaacagatttttttagacctacacattttcattgctttcatttcaaagtccaggctgcaattttgaatggataagtaaaagtatcgtaaatactatgacaataataaacacatgttcttactatgctatgagtgatatgacttaacctatgtgtattcagttgagagatgggtctcgttttattttgtcctacttgctcccgcatatagtgcatgtagtgattttcttggtacatgaaaacattgcatatctaggtaaaatgttaatggccaaggccaaggtgggtggatcacctgaggtcaggagttcaagaccagcctggccaatatggtgaaaccccatctctacaaaaatacaaaaattagccaggcatgatggatggtgggtgcctgtaatcccagttactcgggagggtgaggtggaagaatcacttgaacctgggaggcggaggttgcagtgagccgagattgtgccattgcactccagcctgggcgacagagcgagactccatctcaaaaaaaaaaaaaaaaaaaaaaaaaaaatgtaatggccattatgtttttctttttgtagagtaagtgtatcttatgtatagtccacttatagtttgagtaactcttgttttgttgtatgcagtatgatattcctataattgattcctgtgcatattatattaaaatgattgcatatggattttccctttggatcatgaagagcatatgatatgatatcagatttttgatctttgatctttgttctgtgtaattagctatcaaaatggaactaactaattttagattattggagcattaggaaatggatgaaccactaaatgttttcctacaaagcagagtagttctaggatgtgcgaacagaagggaagactggggctctattccttggaggcacctccttcctaagtatttgcagacctatataagtgacctacctataacttgctttttaaggatgatatatgtaattagataatttttgcaagcagctggtcttgggagttaggaaaaacagaacaatataatcatccttattattaatatttcatatcaaaattttttagctttgaggctactcaggatttttgggcagataatactatgcttaagtaaattttaaactaacagaaagcgatcaaactgattcattgttcatagttggtatcagtaagttggagaagaaatagaaagagttcagagctcagtgtatttaaacctggaattaggtcattcttttttatctagaaaatcccagcccatgtggccttaaaaatataattttatttttaagaaaaaaatagtttttttgaagactgcttaaaatatatttgtcatttaataataactgtctctctaaatggaaaagtagccagagttcctggaaatcagcagttagttgctgtggattagctgctgcttaaagtttcctaatctgtaatgtaaatgggtaaaatcaggcaaatcaatttctaagaacttcataactttgtgatatcaaaaacaagcaaacaaatacaataatttttcttaaaatgtgagatggaaagaccttcaagaagacctagagtcatttgttggttcaagtttgcccagacgttttgataatgacaggtctcctctcctgacctgtggtacaatggtcaatctccacactctggtcaatatctatcaataacataaaaatgtgactgttcttggatcctgtatttattccccaatattaggcctgagagtgccagaactggcacttaatgtattgatggaacaccaaggagagaataaaaatataccgaagagcactcgacacaaaaaggatacaagaatttactgaatagtgaacaaattatatgcatgtactgaggttcagtttttctaacctgtcaaaaaacaaaaaaaatgacaaccaaacaagaaaaatgtctctaatatagacactaagcagcattattttaatacatacatatatgggtacattcttttttgctcattaagaaccacaggggctgacgcagtggctcacgcctgtaatcccagcactttgggaggccgaggcgggcagatgatgaggtcaggagatcgagaccatcctggctaacacagtgaaaccccgtctctactaaaaaaacaaaaaattagccaggcgtggtggcaggcgcctgtagtcccagctacttgggaggctgaggcaggagaatggcgtgaacctggaaggcggagcttgcagtgagccgagattgtgccactgcactccagcctgggtgacaaagcaagactctgtctcaaaaaaaaaaaaaaaaaacccacaaaatatatatattttatacatatatatatattttttctctctcctctgtgtgtgtatgtatgctctctctgtgtgtgtgtgtgtgtgtgtgtgtgtgtgtgtgtgtgtgtgtgtgtgtgagagagagagagagagattcagattgagatttaaagcccggggctaggtagttttattctaaacaaatatcaaataaagaattgaactcaaagtgaaatactgtatccgcataaaatgtgagccagagaactactgcctaaaaaatgctgcctcacttggaacctaaagctaatatcattcatttatttagttagctttatagtatgttggttttggatttgaaatgattagaataaatgcagatggaaagtgtttttacagtagggaaataattaaattgggtgtggagactttgacaagagaattcttgaagtgacatatgtgtatcagcaaattctttttcttgcttggccttaattcttttgggaagaaatgcttgggctgcagatgtaatggtatattgggaaaaagaatttacaaacccatgcagtataagtacggcaccaaactatcttctttgaaatagcattacatgtatatagtataagccagatgtcaggaatagtattttttccctgtttcctaaagctactaaacttgatgtcatgtggaaagggatgtgatgtgctttgtgtaaaatttgacattccttaaatgtttttctttttttgctttcctctgtgttgctaaccatgtccaggatgtactattgttcctaaactaatgttaaccataaaatgaatttatgagaaaaacaaaattaatatccttaccctaaaattaagtttgttactagatagaccatcttaaaacataaactgaaatgatattttgcattgatgtttttttctgtcttcatggtgaatttatttgtgtcaatttgggtcccatttgactcatcaccgaaaatacatggttcttgtttaatgtaataaatagctcctggacaaatgaattttacagcagaagtttccatgaatcttatttttttaactcctctactttcagtcctttttatttctgataatgaggtgaggaaataatttatttcattcttgtattgcatactttacaaatatcttagggtgtctctgtgttaaagtagtccctataacacacaaatgatttctctgttgaatttctatttctgtttctctttgcttttcctctcttgggccactgactagcattttcaccaagaagagatcaaagacataggaaggaaataaaactcaaaataggaaatttttattttatactcgttttgttaactagagagtgaatagaaatagaaattgtggtgaaattgggctgtgaagtgacttatcaggagttaagccagctcttgtcctttagtagctttgtttcatcctggccacagtgtcaccagtagaatatagtaaggagatctaaggagaatcttaaaatataggaagaacttgtggtatgtatgagctaagatgatagtttttccacatggagaataacaaaagattctgtggatagtggggaggattgactcttctccacttcgtaagttaaagaaattgagtttgctgagaacttaatgattacctgaaattagtaaaggccttggcctgtgtcttcttgccctagtctttactaactttccaattattaagagaaaggcaaaatgactttccgttttttaaagaagcattggaattagtttcagtttaatatgtaatatttaagtgagcctcagggagtttatctattcaatgtctacatgcaacagcttaaaaaaatgaaattcctaactcctattatgtagacagttgtgatttaataatgtttaatgtattaaattatacatagttatttgctagtaaaataattattgcagtacttccggaagactgaaatgtttatctgcatataattgcctgtctcaaaattaaatgctgtattgttttcaaagcagggaaagctgagggctcctttgtgaaacatgcatagcatttcactgtgtcaaatcagaggtccatctagcccaatattctgtctccaagaagtaccaaaaatcattactgccagtttatgtttttctcaaacctggaaggttggaatatagttctaaataccttaaattctctcaataacatgatctggttgtacagaccacagaattgggtaaccatttcttggacttaattatattatcatatattatctttttgactaatgaacccaggaaattactaatcacaatgcaaagtaataattaattcctttaattattaaaaaacacctctttcaaattataaggcatacttcccctgaaaatgatagatattctaatagttaaatattctaagagtgtgatttaattctcatgtcatttatttaacctgagtcatattatagcttaatctttttcttcccaatttggaaagtgttttgtcttgtttcaaagtctggcctcatgtggatgcttctctagtcttttcatcatttttggctggcatttactggagattttcagaatttgttttaccttttctggggtgtgctgacagattcctgcagatatggatgcagatggggctgtgaatgagggtgaatgatgtcctctgttttgctgttaattgtgtctttgatagctgacctatcttgatcctttgttcatggtggcatctgtgctgatttcctcagataattgtctgtggtgtttgccaaatcacttatttcagtgataattgcagctttaaaaaacagtctcggccgggcacagtggctcaccctataaacccagcactttgggaggctgaggcgggtggatcatctgaggttgggagttcgagaccagcctggccaacatggtgagaccccgtctctactaaaaatacaaaaattagtagggcatagtggtgggcacctgtaatcccagctacttgggaggctgaggcacgagaatcgcttgaacctggggggcggaggttgcagtgagccgtaatcatgccactgcactccagcctgggcaacagagtgagactcggtctcaaaaaaaaaaaaaaagtctcaaaacatagactattgtttcctaaatgtcttatcacatatgtgcccaatcgacccttccagtagctcattgggaactcctggtaacttatctttattggccagtgtagctgagtgttatttttgtacactttttgatttctttctgcacttcccctgtagagttcatcattttctaagattaaatgaatgggcaaatgagaataatatagtacatgtcccttctcattgttactcagagaatagttgcttattatgtgtttaaattgaagaagactcatcctaaaacccattaaggaaaaggaataggctacaaccaaaaggaggaagggcctcacagtggcatttcttcattcataccgtgtttatttaacctttgcctcactgtcttcagctccctaggttttattcttgatgcttttgcttttctttcatttcatactttttcatgtataatctctgtttacaactcttgatgtggcccacagtgcctagcatgctccgcccacgtccttctgcttctgctcggtgtgcatgagctgctgcttgttaggaggtctgcattcaccactgctacttgcctctctccacagaggctgggactctccttccctttcagtttctcctttcccctcccacctttgcttacctacatcatttcaaatcatcttccagatgttttgcgcccgttgcagcactctccccttctctaattagagacaacttaatggttattcatttcactagtatagagacttagagcttgatttcataggtcaggcatctacaaactgtgatatgaaaggagagattgctagggacagacctttaagtggagtggctgccaaactcctgaagaagctcagtgctttaaggaagccttccctatccagccaactagtccagccccactgtcataaggccacagggtaccatgttcttctccttcagagattctgcatgtggtagatgcctcctcagttcaccagactataagctctacaaggttagagttcatatctgcctttgttcaccattgcctcattagtatacagcacagggcctagcacatagcaggtccccagtacatatctgttaaatgaatgaataacttccaaatacacaaatctggcttgcaattgctcctggaatatctcttttaaccacaaatccctagtgtaatttagttagggatttctctctgccatctcagctccacttgtgaaaaattgtaattacacttctttccccagggtctttcctttctagtgataaataattctctcagttgtcctgtctcagaacttttaagcaatctttgacttcttgctccctttcctaatcctgctggtcatcaagttgacattcagctatagcccccccaattttctatgaaaccatgttttttgaattttcctctgccgtgctttttgaatctctttatcctgcctcacctcactgccactatgaaagttcagaatcttgccacctcacctctgaatgaatgcagtaggtcttcacatggcttacagggtctagaccctgcctttctgttctgccaagactgtcagagagcctttcttaatgacactttccatatggtgttgccgtgttggtcaattgatcaggcagtattcagtagacagagtatatacagaagcatgagcaaataaccttaaaagtttatatgtgcaggaagtaaaaatgtgaaaatatactttttgtgcctaattactgcccacaaaaggtcaggggttttctgggctacctgttgtccactctcctagttctcttatttttatttttattttttgagacagactttcgctgtgtcacccagactggagtgcggtggcagaatctcggctcactgtatcctccacctcctaggtttgagcgattctcctgcctcagcctcttgagtagctgggactacaggcgcccccgccaccacacccgcctactttttgtatctttagttgaggtggggttttgtcatcctggccaggctggtctcaaactcctgacctcaagtcatctgcccgccttggcctcctaaagtgctgggattacaggcgtgagccactgcacctggccctatttcccttattttactctgttaaatgttgcttgagtctcattttgtattctacctatcgttccaaactgacatagcagtgcatggctctaacattagtcccaaaacattctaggaaaaaatatataccagttacataggtaatgttttccatctcccctgctgtctccatttcctacatgtatcatagagtaaaatcatgtatgtgtgaactctgtggccacccaagggcatctatttttaggtaatacttatctggtaagttctacagatgaaatcatttcattagagggatagaggagaataagagcaccagagtgcagtttgagcactgggggttagtgtggttaggaagggcttcatagaagattggggggctatagctgaatgtcaaagaatagataaattttgaagaaatgaaggaaagagaactcacagtgaggtacaaacaaacaaacaaacaaaccaaaaaacccaggagctgaaattgaaaggtatggtttttggatagtcccacttgatccatgtgaagaaatagtagatgagattagaaagttagggtaggttagcacagcttgtggctggtttagcttgctagttgttttagttaggactcttattggttgcagctactgttgtcagccagttgaagcagaaaaaggaagttttagtatctattattttttgggttttgtttttgttttttttttttttttgagatggagtttcgctcttgttgcccaggctggagggctggagtgcaatggcatgatcttggctcaccacaacctccacctcccaggttcaagcgattctcctgcttcagcctcctgagtagctgggattacaggcatgcacctaagcaaaggattaatttcttaactcgggaaaagagggcttgctgaatttcagaaggtatgggagacacagattatctccctgttcatctcttattcatctctgtcccctttgcttcaccgttctgtgtctttatctctctggctgaccagctttgtttttctccaggccacacaacaagaggaaaatgcccatgtgacactgctcctgattttatacatttgacttctttagccacctagataatagattcctctctttttcattatctcagttccagatttctagggacgggaatgttttcagagagtcagggtcacaaaacagaaatgtggctttcaggagccacctctgggagcctatgaattgggttgtgaggagctttcaagcaaaggttgagggtagataacccacaagtgccctctactttctactccttcccttcccaggacatttttaacctgccagttttatccagcacctacccctccctcatccttcactttataatccagcctatataaactagcatatatcaaggaaattgaaaatgcactcccccctttcctaattagagacaactcaatggctattcattttactactatagagacttagagcttgatttcataggtcaggcatctacaaaccgtgatctgaaagtagagattgctggagacagacctttaagtggagtggcagcgaaactgcaaagtaagaggaactcagttcccctctttatgtaaactcttgtaccaatttttggcctccctatacccatccccaagctgaccagttcctattcaagggacccagtcttctcttcggctctcaaataccaattcaaatgcaaattgctttctccatttcctgtgtgtagttgttagataattgtctgactaatgccctcaattttgccaagattagtgtctaggttttagctctttaaaaaaaaaaacaatgttcaaacatacctgcagaaagacagtcagcaatcaataacctagagcccagatttaaaacttggcaagctctcttacaggtattgagagctcaccaatggatttctgatcaggtaatgatataattaaaattgtactggtgatgacagatcaaccgtgactcaggtgcttggaggcagggagaccagctatgaaacttacataaaaggctggagagacacttagcaaattgtggttattttggggcggcattacacactatttttttcatagcttaaacacattttccaaattttctacagtggatacttatttcttttgatttaagaacaaatacatgcatataaagcctgaaataatagatttggcttgaaataggcatggtggcagggggaaggatctggcctcttctttctgacagcttgcattgttctttagagatgagaaaatgcctgtcttcgccacatgtattagtgattaattccagaaaatggctgtgttcctggtcagcctctcctacagtaaactgagctcagaaatctttgtggttaacagtacaaaactcgttctggagactgttagctgcttgagttaatttggtcagagtaattcctcatgtaaagcagcatttgtgcagtgacttctatccagaagacctaaggagctaggacaagactctgctttgcatcacaggggtaattagaatgttgtcgattgagggcaacaaccatctctgagtattctccaagattgcttaggggttattaagcatcttaaatccttattttgcacttagagcatctgtagtgcagaaattctagactcagtgtgagtagaaacatagaagcttttgtggtccacttcatgcttggccaactctagatggtaatagaaagggggatatagtggttttcctctcagtagttcaattaaatgcttattctcccccttaaaggtactatttattgagcatctactatgggtcaggcaccaaatgcttgagatacgtcagtaaacaaaacaagagcatacatggtaaaggagtatgttagaaggtaatacataaccaccaattaaataaaatagggaggatcaggaacattgcggaggggaatggttgtggttttaaattgggtgatattttagcaaacttgaaggaaatgaatgagtgagccaagcagagagctggtggaagtacactcccttcagtaaaagcctgcgtggcatgttcaggtgacagaagagggaaggacagtgtggctagaatagagtgagcccagagttcaaaatttgaaatagcaatattctagaaaaagctactatttctagtcttttccacattttggaaatatttacttgtaatttcaataaaccattttcaaataaatgtcttggactaattgcctcttttgtcactgctacaagatggggctgatccccaaccaggaacacctctctctgccacaccacacacatcttctaagactagggctctctcctgatcagaaggcttcaagattcttcaccagtggaactcgaagtcttaaggctttttctaaagtagtgttcttaaagtttcagtcttaaatatagatgagggcttaatagagattccaaaattttgatttgtaaaggtacagagattctgcttattatcttacatttaatcatcacatttcagttgtcttagcaacctacacagtattctcatttggttttcaataagccaatggatgaatgggttctttgttcacgtgtttatgaatttctatcacctgatacttgaagtctttcaggcatgcttactgtgccaaataactgtctttatattcctaaaattcatgtagctaaaattaagctctactttttctaaatatttacatttccaatgagagaatctccaacataagaatagttggaaaaatgtatatataagcttgtgttttttttttttttttttttttttttttttttttggtcatcgaatgatttaatccactcttaaaattctagaaaaagttatatcaaacaaatacttgtcttgcttgggtagtaagagccaaataatacaggtatctttagttcaacagcggcacctttataagcttgtatttaaaaaatacttttgctttagttataattaatttccaaggctccacagacttgatggcaaactcaattgtctagcaaacctcacccatacagacatatttgagaattacagagagaaaaaaaaaagcctatgacaaatctaggcttcatagaggtatctctgagatgttgtctcagaatgttttgtcttactgaatgcacagttttaaattctaagtgaaggaatatatcgaaaatgtttaacaaagtacctgccactgtattatgtgaatttaaatgactgctcttattacaagtaggcatagtgatagtattgacaagtgacagtctaatgacaaaaagacaaaaatagcgaagcataaatgtaagaatgcaaaatgcttagtcacatgagatattttggcatgaacactggaagcccagtatgctttagtaatccctgaggacattgatatgccttagtactgtgtgatgattctcactagtgttggtcaccctgattttctcagtaaaagttaaattacatttaacatcataacattttagaattatttatatacaaaatgatcccattaaaatattttagaattatttatttacaaaatgatcccatttaaaatatgctattgcttaaaataaccacagctcagcaacacacaatatttgactaaaccataatgaccaaattccttgaaggttctggatagatgaggttatctagaattattcctttttcatgctaacatgagcaactcaagcatctggatcacttagggatttttttttttttttctccaatccaggtcaggtacctagcacagtgtgtggcacatagtaggagtgcaataaatatttataataggaattggtgaatgaatgaatgaggagctagtttttatattttggggagagaagtggcaagttcttcaagtctttacctacactagtcgcactaccgtatgccatataagaaggtttaaatgtcatctgtaaataagatcaaatctaaggaagacctctaagtttgggctgtggtggctcccgcctgtaatcccagcactttgggaggccaaggcaggcagattatttgaattcaggagtttgagaccagcctggccaacatggtgaaaccccatctctactaaaaatacaaaaaattagccaggcgtggtgttgcgtgcctgtagtccctggtactcagcaggctgagacagaagaatcgcttgaacccgagaggtggaggttgcagtgagccaagattgtgccattgcactccagcctgggtgacagagcaagacttcgtctaaaaaattaaaataaaataaaaaccctctaagtttgctctaacaagtagagtaactctgtatcccagcttaccccatgtagagtcccagtttacacttggtatctcagcatcccttttggatagagagtttactttcaagaaagtcttgtttgcatgatgaatttaatggttattctaattaaagtcatcacgtccttaccaaaggtttttgaaataaaaatgtaataatcaagaatttccctttttctgtctcccccttcttttctcccttccccttctttcctatcttaggatgacttttaccatgggcattgcacttttatctttaatacaagacaatatgtgaaaaagctctttgaaaggaaagatgacttgggaaataattgttccatttttttaggcttggcttccataggaagctcttctaatgcattctgtcatgtgattaatttgtcaaaattccatacctgtcagaaataatgtaatcttttcaagaacatagacagtcttattacatgaagtgatgtaatcctatactatttatctcctctgtattcctcattctcttattttctattattcttcaaatagaagagctaaagtcccaactaatggggatgttagagatcatgctagagcctgggaaaaaatgattaatgtggtttggtgggatgccaagacagatgcgccacaccctgctcggtgcaacagctgttgtagtaaagactgaaggcattacataggagcatcatgtgtgacatcgaacgggagacaaaatagtttactgtggctgagacactgacatttacatattcttaaccattatgttatgtgtgtgctgttggggtagttattatcctaggtttttctcattatttataggtgtttgttttaaagagaaacaatgatgaaaaccatggtatagagaaaaaatgaggagagggtgtgtttgtgtgtgtgtctgtgtgtatatgtgcatgcatgcaacatgactggaagtgtggattacccattggtatttctccaaaggggatttagaagggttaattctttctgcgcagggctgacagcacatgacacaactatccaaccatgccttcttccagatctcagtagcagttccagtcacagtgagaaccccaaactccctgccgtgttttcacactctcctggctacccaagagatgcatgattttcatcagaggaagatactgtgtatcctaaaaattcacatctcatggacttaaaaagatctggaaaatgcaaggcttagaaaatgttagtgaagcagacttttaatggcccagaatgaatcactcaacttggctggaaaaagcattctcttctatagcttctgacaatgaaccatggcatggctgcccagatacttaacattgcaatgagggcatccacatgacagtataggcagcaaagcacaacccctcaataccttagaaaagcaactcacagttggtttcccagtaacatcatttgactgtaggtgctgcttagaaagagtcaataagctccagatcctgaattcaaagccttttcctgacactggaagaagagcagtgtgaggcttcaaattgtttccaaaacgcctacatttccagcatcttttctctttccattccatttccaatcagctgcattccatctagcaaattcttgatattagggcatacagatttttagaaaattgaattttatttcatggaaaggttatttttttgtgcagttagaattagtgatctttgaggaatattgggaatagttggcttttcacatattgataactgcttcttgatttattgtttatcagtgctctaaacagggtttggaaaatatttggaactgattccttttatttgcatatatgatatatccccatgtgtattagttttcacactgctgataaagacatactcaagactgggcaatttacaaaataaagaggtttaatggacttacagctccacgtgcctggggaggcctcacaattatggcggaaggcaaaaggcatgtgtcacatgggggccgacaagagaagagggcttgtgctttttaaaaccatcagatctcgtgagacttattcactatcacaagaacagcatgggaaagacctgcccccatgattcaattacctcccactgggtctttcccacaacatgtgggaattcaagataagatttggatggggacacagacaaaccatatcaccatgtttcttacagtgcattgttttgtgtgtcttttcccctatggccatacattgtgaatccatttagttaaagcaactgagtggccattaaatgttttgtgctaaatgtaagagataaaactggaaaaaagaaaagggggggaggacagtgcaagatatcagcccttgttgcctagtcaagaaacagaggaatcgtgaaagaaagcttattttccatgtttgcaattcctgggaatgaccaaactggttttgcaaaccatgtgaattgagtccatgtgtatgctaggggtgagggagttattgagactagtttatttgagatattctgggtgtcttttactttctaaatattcttactccttttcttatgggggaaagaattttatgcaatagactttcattaaattgaattttcacaaccaaattgaagctgttatcagttggttgaactcttgctaactgaatgactataaggacaggtttcaatttcttcttatttaaagagtctgatttttttcagctgctattttttttcctcatattttcatgttgcctttatccagttggctgtttacattgtcagtaataagagttagaacttctaagtgtgacatgtgttttttatttcaaggacttctactaaaattgtttcttttgcatgcatgctatggaaggcttgcttaaaaactttgagaagactctaagtagagcccagaatttcgctaagaatgtggaccttttctttggtaagttggccatcctgtagaaagcgaatggcagtttcctaaagaaaaccctaatatggcttctttgaaaaaaaattgccaacatgcagtgccaaatcgtatttagtaaaaccttgtccccaaataaaaccagggaaataggcacaaagaagagaaacaggatacatcagtgcatgttgcaggagggttggatttgttgaaagattaatcataaagtatgtggacaaaagtgaattaagtaagtataagatacaatgtcactacctatttattcatcttttagctaaatatatgccttcccatgtctgcggacttttcctctcttattttgaccagcgccagctcacctctgctggtgcccacactggatcatgctaaattcaggtcagcatgcaaagggaatcagcctgcttagctagtggattttctaggcataatttatacatacataatttttagtgtcaggccaagcattttcatttttataatttcttctttctgatagtaaaccatttaatttggagcaaatataaatgattcctaatgaagcacaagtgaacagaaggaaattacttttgttaaactagtacttcaatgtatttttctctccctttcaagtagaggatgactatgagaaactgatacacagtattattcattatctggttagttcactagatcaagatgttgatgtgtggtgtagaaaggtggaaaaagttagagagttaaaaacaggttgatacaagctgttgagaaaaaaaaatacaatgaggctacttatgttttaaatcaagaatatgttttcagtgaaaatactctctaatgattcacagaagaatgtcaacttggactacatctgctgaaaatcaaagtgtaagtagttatatggcattgaattctggcaggagtctatgctgtgatatgaaacgaaagttcccatcaaatgacaatactttatttttctgcctttaagagtagttttctgactttaaaaaatgttaacagcattttttctccttcacattacaaagataaattggcaacgtgcctgtatgatttccagagtaggtttcttaaaataattttgatgggaaggtaattgtttaatgtcagtgtataaagttctcccatagtttaaacaaaattaaataagagaaaacacaaagaggaggttttgtataattactgggaaaacatagaaggaacaattaggtagttagacctgaaaagaagtgaactagggttttcaaatttcatactatttatagggttccttgcattattctctgacatcacttgatttttttgtttctgagtttgcaaatcagaatagcagcatatttatccattaatacatatttatagctttttgcacttatatattttctattcaagagtttcatgataaaatgaaatacactgaatacatattatgtgcaaaacaacaccattacaaaagtatacactgatacagatgcgagttagaaaattatatgcggtggggcgtggtggctcatgcctgtaatcccagcacttctggaggccaaggtgggcggatcaagaggtcaggagttcgagaccagcctggccaacatggtgaaaccctgtctctactgaaaatacaaaaattagtagggtgtggtggcgcacgcctgtaatcccagctactccggaggatgaggcaagagaattgcttgaacccgggaggcggagggtgcagtgagctgagattgcaccactgaactctagcttgggtgacaaagcaagactctgtcttgaaaaaaaaaaagaaaaaaaaaattatatgcttatattgcaagccgctttgcataaataatctgttctaatataggattcatttagcaaaattcatgaaaatagacatagtgtttgtgccctgaacatcatattgaagggtatcccagtcaattaaaggatagtaataaagtataaaaattattattgattaccttatttttttacagtgttatttttaaataccttggagatatcttaaattgttatttagaatgtacaatttaacataaatttaactgaagttgggacctgcttttttatgttaaattgtagactttttcttactcaattgccataaattctaataaattctaacttttttgcataacagtggagagaaagtttgaaatggttcatatgagcagctttgattttggaatgagactggggacacagctcaaggtttcctattttctcctgtccctctcttctggaatcaacccaagtgcccatagtgaatgaatcggtaaagaaaatgtggtatatgtatacaatggaatattattcagccataaaaagaattaaatcctgttatttgcaacaacatggatagaactgggggtcattatttaagtgaagtaagacaagcacggaaaagcaagtatcacatattctcactcctatgtaggagctaaaaaagtggatctcataaggatagagagtagattgtagattggtgtttaccagaggccaggaagggtaggaaggaaggagggatccaaagattttggttaatgggtacaaatataaattagatagaagaaataagagctggtgttcaacacgtcagtagggtgagtatagttaatattaatctattgtacatttcaaaatctctaaaagagaataattggaatgttcctagcataaagaaaatacaaatatttaagatgatggatatcccaaataccctgatttgattatatgaatgtatcaaattatcacacgtaccctcaaaatgtgtccttctgttacgtaccaactttaaaaagaaaataacatcatataacaactatagcccttagatgcaaaaagacctgagtgcaaatatcattccctttcttactaacatctaagtttcatatagtcttttagcttcatcgtttaaccccgaaatctggtaccatcctcttctattttcttgatgactaaattagatggcattatgtgtactgtttgacacatagttaacactcaatgaaagatagttatcttcagcctttgtacaaataactctagagaaatattagttgacctattgaaatgtggcacaatccagggctcacccctctcaggatcattagcattagattgagggtatttccctgcatacctctctctaataccagaaactcaatattttagtagtcacagggaaaagatgatagcaacagtgccatgggtgaactgtactctttttctgttctttgagcataaccataatgaatcaagaggaaaaggggaaggaactgaggagttgcccaccagctgtttctactgactgtgggaggaatttcctttgctcttttgttctaaaagatatctagatcatttcagcaaaaacccatatttttaaatcaaaaaaaagatctatatgattctagggacacagacacacacacacatacacacacacataccccttctttacaccattgtagaattgtaaaattaacacgtacagctaccaaagattgcatcatgacacagaaggatttctaatggcagtacttaatacctctccaaaataccttaagagggaaaaccactttggtacatctgttgtcactgagtggtgagcacttcttatattatctttattctctaagtagttgctcttcgttatttattcattggctctctaaatacagcacttaccctttttattccctgtctgatttctaaaaatggaacattcataaaataactcagtattatataaaaatttctaaagtgagaatctagctatatatctggaggcttaaactcacattgttttgttaccaacacaaatgttgaaatctactgaaagagtgtagtcaacttttccctgagcaaattcattttgatgaggcactgtgggaatttttgtctggaactgggtgtcatatgcttacatctctgtctaggctactgattcagggagctgtgcaatataaagatgaaatgtgatacacacacacacacacacacagccaggagaaataaaatgtgaacccaaaagaataacagaatgaatctatttgcaaatgtagtaaattcccatgtttatagagaaacaagagggagggtaacaggggacaagaccagtaagaattagggaagcacatggcttatagaattgttcattatctgatgtttccatgaattgtgacactctaggagtggagtctcagcccctccagcacccactctaccaacttgggcccttcagtgcctcggttttatcatggggctaataacaatgcctgctttctacaattgttatgaggattaaattagctataactaaaacacttccagcagcatcaagtgctatttttattatttccattactaattttgtgtttatgcattttgtggaaaaggaaacatgaacattgtgtggttcaaatagccaaaaaaaaattaaaatgtgattacactaaattagaatttatgtgattttattgcacctgaaggttaacatgattctgggattctaataacaaaaaagctttaaaaccatcttgatttgtttatacagaaaaacaggaattggcctgatagagaatgaaaactatgaaatagctggctgtcttgctttattgatatttttcacaggctttatttaactcccaaagacctagcctaaacaatagtatttttagaggacatagtcacataaataatctttctcattttgctctaaatgcctgctttaaccacagggggaaaaaatacaactttgttcttgtatgtttaagtcaaagatagtacacatagtatataatgttaattccatattaaaatgaccatttgaagaaagtgttcattaagaaagattacatttacttcaaggaattttcttgatatcaagcaatatttatttttcataattgagcttatgtgcctaaaataatccactcaaatctggataaatcatagtcttctatttgcacattcatcaactaaactctcatataaggaaggaagccactctacaagtcatatttctgaactgggaattatcttttcctgtcatgttttttgtttttgtttgtttgtgtgtttttctttgagatggagtttcgcctttgttacccaggctggagtgcaatggcatgattttggctcaccacaacctctgcccccagagttcaagcaattctcctgcctcagcctcacgagtacgtgggattacagttgtgcgtgtgccaccacacctgggtaattttgtagttttagtagagatggggtttctccatgttggtcaggctggtctcaaactcccgacctcaggtgatccacccgcctcagcctcccaaagtgctaagattacaggcgtgagccactgtgcccggcctcctgtcatgtatttttaacaggagtgtctcaggaatttaagccctttatttggactgactcccctgttcacttatttttctcagttcagtgagtgttgccaataagagagagattagacaaaaaagacaaagcacatcatctcttttctaatgtggatgttactcagtgatgaggggagagcagataagatccccagttaccaaagcgattaataacatcattctgtccattcacactcattcagccagtgtttattgaagagctttgtttgtctctgtcactgtgatcctacaggaaagacaatacctcctcttaaccaaggttgacaagtagctactcctagtttaataatttttgtaaaactttcagagctgttttttttttcgtattacaaatttactacattggatctactacactgtagattcttcagacagaaaatacatatggtcatgctaaataaaacaaaaataatctctagtttcagcacccacagtcttctagtcatagagagcaattccagttgttggcttgaatagtaaagcttttttctaacacaaaacatttgagactagtaacttcctatgcatttagttatcagttaatgtaagtctgtgtgtaataaatacgactttcattgagtttcgggatgtccttcgtgttcacgtgtagccctgcctcatcacccaccttaaagtggattgcttgtatccagtttctagtctccttggctcttcgcatgaggagagagagaaatgggaaaccctaattccctgttcacttgattcaacatctacgatttcattctattttaaaactctaccctggcagtaccctgcttactcattcgtagctgccttcagacatgctattctctgcttgaacatagtcgctcttagttcctcaacttttattttctcacttaagagcctgcttcactgagaatcatttcctagtgtcatgattctgaggtgccttttccatccactccttcaggctcctgtgctcaccatgccatggcctcaacacactgtgctagaatattgttttaccccctgactccctaactggactttgagttggtaggtagcattttatctgtcttccaaggatcttagccatctcctggcacatggaagtggtcataaaatatctattggatatatgaatgaataaatggactgacatcttgtggcaacatcctttcccccagtaaaaaccagaatggaatatacttcttatagtccagtggttctgactgaataacatatttattttggtagcagaaaatgccattgtttgggaaaactcaaggccatctactaacttctttattcttgcattgtcgtgatgatgtgactgatttccacagatctcataaaaatcattctcggtattctatattctatttcctatgtatgaaaggtgaatactcttgagtcattaattcaacaaattaattcaacagttatttattgaatgtcaattatgttccaggcacaattttaggtggggctacagcaaagtataaaacagacaaaagtttctcctttggggtttacattccagtggagagaggcaagcaataataaagattaatatacaaaacagtatattagaggtggtaagtgcaatggagaaaaagcaggaaaagaagagagagaatgaatggggggaagggggtgacaattttgcattgcctggccagggaaggccttagtgagagctgggtctcactaaggaggaaatgagggctgggtcatgcagctgtctgggagaagagtcccaggaagagagaacagcatgtgcaaaggtcctgaggtgagagcatgtctgttgtattttaggaactgtgtagtcaactggagagcagagagaatgctgctaaattctattatctatctatctacctacctacctacctacctatcttctatctatctatttagtaacattgtatcagattactttatatatatattgtataggctgtcaaacttcattcatatttttatttactaattgaccaggcagttatttaccacctaccatgggccaggttctatcttaggtattttaaggaaagtaaaatctattagatatagttcttgcccaaaagaagtttacgaaataatcagagataagacacatacaggactcatgacacgagatgagctctcataaggaaatgactactcattggccagagaaacagctagagaatgacatctgggcttcttggaagagatggcatcaaactgcaccttgagaggcatacaatgccaacggcacatcatatgcaatggtgtaaacgggggcacacccactgagtcactggcagtgcaggatctgtgaaggcaggagtgataggtaaggtcaaaaggaaagctttggtcagcatttgaaagcctttgaatgagtatatattttgtgaattctcttcattaaaaatgttctaagatttgtcatgatgttcagtgccttggtcttcataattctgtgttgccctcacctaattgccagcatattttagttaaattcaattagatatttcagttagctatatacatttttcagattgaaagctgttaacaggttgtataaaacctgtttcttatactttaacaaaataatttccaagtgtactatgcgatttttaattctagaaggttttctattaaaatgaaaatgaaatgtatgaaatctgtctcttattatttcagtcttctataggttttcgagtgtaaattagggagaaatgcagaataatttaaaattaaagttacttatagagaaagctgaatagaaaaataatgtgtatatagcatttgtttttctagaataaagtaagcacttatttagcttttaggtttcaactatattgtctttctcttgagcagattttacatagagctggtatgacatatctcagtgtaatacataatatattactaatgtctttaaattcagaagataagaaaaaactttgagatcaagtttttaatttgggaaaggtctaattaaactccttacattgggatatttgagttaattgttaaagtgctaattcagtttatcaaccacttgttatcacatagttttatcaacctacactgtgaaataaaggagtctagggttttttttttttttttttggaaggaaacgtatttatcgaaagagatttatttagattattctacatattctctgggaatttgtagtgtttcttaaagttggactaaatgttgaatgggaggataggaggagaaggcgatggaccctacaggtgcaaactctttccaaatcaccagggctttggctgttactcactcacccccgccttccttgctcttggagccttcagatttttccccagtagcttcaccttacagcaagcttagcagaagcagttccacagcatgccaacccccctcacttttgaataatccagagactccctgtgccacagataacatgcaatcataaatcttctcagcttcagcagttttcgtcttctaaattcaattaagttgagcccagtaagaatttgtggatcaaacagtgagctctatatagattcaaattcaacttagggttctgatgtttgttactaattttcaggaaaaaaaaataaaagaaaaatactgctgctattttctaaagcttccttataaaggtatttcttattgaaaccaactcatatttctcaaattcctagaccactatctgtcatgcaatttgagtctgttttccaaatggtagagtgccagagaatcaaaagctgcttgttttgcttgcctgattttctttagcattctttggaggatttaaagtatggggggaagaaaaggaaacctgacttacattaagagctgaacatgaaaaagggattaggcaaagctttaactttttcattatgcaaactttcctgaactataatacatatctttatagagttttattaattgttggggctgcaatttggattttgctaaaaagtatttactactaaaaacttcaaggttgttactaaacacatcaatcattttacatttggcccctttgcatggtgaacagaccgtccttcctccatcttttgttggggaatacctggctgaaggtctcagtgaaaagctaggaagatactcctcctctgagggagacatctggcttcttggctgattccatgcacacttttgttaatgggctcagattccccagaattcttttatgtggttacattgtagatggactgtctgcatagtcttgtcctcttgaatgatttatgtagaggttaaaaatacatatatatttggtctggttcttagaatctacgcataagattaaatgaatctcagcaagttcagttttggaaaaaaaaattaaatttattattacaaaggcaatctagtagaggatgtagattctcgagccagaatgcttaatttgaaacatggatttgccacttgctagctatacagtgttggacaagttatttaatctctttgtgccttcttggtttccctaaccacatacaggatgataataacaatatctacctcatatgtttccatgaggtttaaatggtttacactgtcaaagcttttaggacagtgcctggcgcgactaagtactcagtgagtgttagtcatgatgtcatgattattcatggtattcaccagacaagaatatgttctcatttgtgtatgatttaggattaaatttgggactaatattgagtggttcaatgctaattttgataaggctcatggaggtgtgtggtcacttttagtggagagtaactttaattcctttttacatagtgaaaaaaagagttcaccttccactcactagacttgattttgcagactccctgttctgatctggattttattatgagtgaaatgaagttagtgactcagtggtccataggtcattagtctgtagctgctctgttacatggaaaagtcctgaatgacaaaggtctactcaagggtgggttggcaaggtaatttagaaacctgtattattcccaaccaaaattggagcataaaaccttctattcactatccaaaaaggaagtgaaatcagccccacatagagatgtatgcactcccatgtttattgcagcgttattcacaataaccaagatatggaaaacagttgaagtgttcatagatggttgaatggataaagaaattgcaaaatatatacacacaaaaaaagaagtgttattcagccttaaaaaggagattatgccatttgtgacaacatggatgaacctggaggacattatgctaggtgaatatggcagacgcaaaaagaaaaatactgcatgatctcacttatatgtggaatacacgtactttctatagatagatagaaattgtatataatttctgttcccttcctcagggtaatttaaatggatattttaaaatagagggctattaagctgttgaaacatttcaaatacaactataacctatgtatatctctatatgtggaatctaaaaataaaaaagtcaaatacatagaaacagagggtagaatggtagttaccagagatgggaggagggatcaggaggtgtggatcaaagtttacaaagttacagttatgtacgatgaataactctagaaatctaaagcactggtgtgaagattatagtcaataatactgtaatgtataccagaaatttattaagagtatggatgttagaaaatctttcacaaaaaagaaaggtgactgtgaaagaaatgatatgttgattgtcttatcattttactaagtatatgtgtatgaaaacatatgttgtacaccttaaatatatgtcctccccccatataaaatttctgttcccttcttcagagtcatttaagtggatatttaaaaaatagagttattaagcttttgaaacacttcaaatacgtcagcctatgcatattaaaattttaatgcagcaaatttactaatatttgcaaaaccagtttagatagagataatatatagatatatagtcagattctgaccatgtcctaagtgttcttagtatctattgatgaatagatttcattaatttaatgagatgatagtatactaaaacttgtctaagtaaatggttggcgtccttgaaacttcctattgaatacaacatgagtaaaatgttcgtgtaaagtaaatatttttcaaatagggatgtgttattggtgtttcttaaatatagattacttgctgctgctttttaaattggatataaactttaaagtgaacagcattgtaggaagttgaagaggaagaaaatatattattttgagaaggtattatttaaatctatataaatccaacttaattaaggacattggataattactattaaggtcacaaatttataaaaatttacaaactaacatgaagtttaacagctttattcaagagtaaagttcatgaattgactgaaagagctcctcctaaccaaaaaaaaaaaaacttctgtatttttccatagaactaaggtcttaattccttctatcaatcatgtgttttccccgtttgcattgaaggctccctactctgtcctgcagcagcagtccctcctgttggcagggcttttctctcctcctgggggcttcctgcagtccacttcaaacagggctctggttccctagcgggcctgttaaattattaccctcaggtgttgattcatccttggcattgttgcattttaccattttgtagtcttcaggcatgggggaaggggaagagagttgaagaagatcaaagtagcagtactgaagttggagctttagaggtggggatgagccctcagcccctctgctgttcctccctgcccttcccccgcctgaccccagcctacccccaccaccccctgactttctgccctctcaagcctaatcagaagacagagggggctatgtgcttgaactgtttacttacaagcactttatttttacttttgcatacaagcaacaaacggtttaacactacatgatgttttattgtatagtacaactgtataaacagaagataacagatgaggacttactatttttttgattcggtggtttttgaacatacactctattctctgtaagacactgtgggggaaggttatgatgagcaagagactccttgttcccaggcagtgctgagtgaaatagttaataaatttttatggagcgggacccaattattacttgtgttatagagcacaaatatgaaactccatgagaaaagcacatcaaagggaaacaccagcatggtttttaaatatctgtaatcagtaaagtattgcatgttctttttagaaaatcagaaaatactcaatttattaaactaaaaacacagtcactctacccagagttaaccacactttggcacatttaattttccatatatgtgtgtgtgtgtatctgtatatctgtctatctatatatatatatattcggtctttttttaatttttgtttcataaaatgtatgactcagtctatcttgctttttaaaaactctacattatcttatgaatatttcaccatagaattgaaagttatttgcataatatatcagagcacataataatttatgtatgtaatcattttcttatttggggatatttctgttcctttcagaatttgtcctcttataaataattcattgaatatatttgtgcataaatcttctcatattcctgattttcttagagtgaacttctagaaatgcaatttcaggtcaaaggtcatgcatattttaaagctcctgaacattttgccagatagcattccatcaagcttataccattgaaaagtcaaagccagcatggcagaggagttcttgtctcatgcctgtcagcaccttgttttgtcattgtaaactataattgttgctgttctgttcccttcttcaaagtcatttaaatggatattttaaaaagtagagggttattaagcttctgaaacacttcaaatacaacttcaacctatgcatatctattaaaagtttaatgcagcaaatttactaatatttgcaaaaccagtttagacaaagataatatatagatatatagccagatcatgactttcatgatgtaagtgttggttaagtaaaaattatgagttttttagtttggcagatttttagtttatttttaaaagtacaatatgcaaacagaaaattatacatatcataaatatgcagttttgtgaattttcataaaactggatacacccatgtaatccgtacccagagaagacatcgaatgtgatcaattcccagaatcctccctcatgccctcttcacaatcataccccttcttcctgactttcaacactgtagatcaatattgcctgtgttctactttgtataaatggaatttcgtgtaatgtatgtattttttgtgtgtctggcttcttttgcttaactttgtatttgtaaaatttatgcattttgctatgtgagagcatgtgtttgttatagtctccattcgtttttgttttgtggccctaatgttgagtgagacctcattaagatgtcatgtcttgatacaccttacatggtctctgaatgatctttgcttgtttcctattcagcatagatctcatggctcaccttttcaaccactaatcttccaacagtcttaagttcctgtctccttttctcttcatcttctttacattgtggcctttggctgctggttgtttttgctgtcctcttcatcggacttcctttggtgggtttgaagtggctatttccatactcaccacagcccagcagctggatgtggtaaggacagggagaacaagcctgcacacctctcttacactcccttccattagataggatgcagtcatgtgtacacttacatataagagatattctaaagtacagaattatggttgggggcccaggagcacaacttaaatcctatttattgcaaaggaagaaggaaatcataaaatactggggataactatgaaatgtgctagaaaactctaccaatttgttctttattctcagctaggccattaatcctgcctggaaattattctgtttcacagacaactctcttttatttctaatagtatttatttaaaaacttctccactctccttaaacttactatgtttcatcttgccttcatagttccacatttgaaggtcctgactcccacttcacggataaattaaaagcctgtctattgtgcctcattctgtaaatggccaattctctctatatatattctgggtctcctaccatttcagaggttaccaaaaacttttgtgttaacccagtgattttttttttttttttgggcctttactttcttaacctctgctaaatttatcactattgacgtgttaactacttcttaaaatattctttttttagctttctaagacagtatctttctggctttcctgccatcttttgattctctttttttctgatatcatgtatatgttcctcttttatgcttatcccttaagcaatgttgcttcccagtgttctatcacttgtatctttttacttaccacacttttctggggtgacctcttccatacccatagcttgaatacccagtatatgctggtgaatcttaaatctctgtttccagcatagatctcttctgtgaagcttcctgctgactgctggatatctctacttgcatatcccttgtgcaccttaaactcaacatgtccagaattgaactcacatgcatctgcaaagattctcctctttcttttcttcttatcttggtgcatcgtgccttcttcatcacagaaatctggatgtccatcctgattcttttttcatgctctctcctcgtatttaatcaatcacaattccctagaaaaatgtgtcttcttaaaatttctagaatctattgactttcattgatctctactgttactactccagttcatacccataatctgtcaccttggtagtggagtctcttgatcatttccctgcatgtggttttctcttctaatctatgcatccagacatgtaagacccccatggtgtggtgcctgctgacatttccaccttcttctccgatcctccatgttctctatgctcatctccatgtcttggtttttacatgtgttgcccactctgcctggaatgtcctacatttctttgtctagcttctgtttagatttcgaaaaaaaaggtaacagttttctctgccaacaacctttgtctgactcacacactcccaaggtttcatgaagaatccctcgtatgtattccccaaagcttgccgttatgatagattacttagaaaaatgtattttaattttctactgatttgatgttacctttactagactttgagcttctcattcctcactgtatctatagatgttatttaaaatatttaacacgagatacagcattggcatggatccaatagaaagaagggtggggccctttgctatgccaggctatatctcttcaattcactggacttcacctggcttcctcctttctgcaccatggcctgaaaattttccccagggggtaaactggagcaatcatagggctcaccagatttgttttccatttcccagggagcactgtctcatgctgcctgatgttcaatatcttgagaaacattattatctgtattttatttagttctttttagtttttttcaggtggaaagataagttagttccctgttactccatcttaggtaaaagcagaagttacaccatgtctgttacctgtttgaggtgggtgacgttatatgtcagttatggacttttattgatatacttagagaataacttaaagatgaagggacatcagaaataagcatttctggagcctttattttatgccatattttttatgtacatgaatttatttcatccaatttggtaaatcttattgtcctgttttacagatgggcaaattgtaactaaccctgtaactaaccaaagttcacaaagctagtcacatccagctttcaaactcaggcttggctgctatcaaacctgcctcctttctcacttacccacactgctggtttggtaaattccagagccttattgtattgacaaaggacctcattactgggtaggtaagtgatccactagagtgaaactgtgccttggtcctgaggtgaagcgagtctttttacctatcttcttttttgtaaaaccctccagtagttccccattcctctcagaataaatgcccaagaacttacaatagtctgaaagtccaatgcagcctagccccccatgatatctttggctccacttcctcctcttctctcactgctcggtcttttctgggctgctggcctttgctctgtctgagcctgccagacgtgcctcctctcacagcctttacgcaggctgttctgtttggaattttcttccctctggtatctgcatgaatgaggattatgagacacatgtgccaattatatttaaattgtaccctgcctcctcatcaacactgtcgatctctgcttcaggttttctcctataatttaccatctcctgacataatgcacaatttacttagttattaaagatgtggggttaaggagttccaactcctctgtgcacttttatgcacttagaatggtgcctagtacattgtaggtgctctgtaaatatttgttaattaattaatgtccaaggtcctgtgccagataagacatctcaaaaaactcatagtctaatgggaggaaacagacacacaacaagtataaaacagcgtggaagggcaatagtagagatatgtatagggaacatcacaagcatttagtttattctgaatggctcaataaacacttccagcaaatggtaaagcctttgctaatcccttaaggataagttggagtctgtcaggtaaaaagaggtaaggttttccgagtagagtgaatctttgtgcaaggatatgggatcagaatcagcatgccttgtagaggacactaagatgtgatactctgggcagtgagaactcatggaaggatgcttgttaagggagtgccatgggcagatgaacctttatggagggaatactcaggctacagtatcaataatggatttgaagaagatgaaattggaggcaggaaagataatggaaggttgttgcatagtacagggaggagttaaaatgcagataaaggactgactcataaaagtttaggaatttaaagcatcacgcttttttatttgatgggatttggttagtgagggaaggggaaaaaccaggtgactccaggagactgtcagtcagtgcctcttgcacattttttttcctgaagtcaattaataactgtctatattgatcaagtgttttgttgatttgttttgtttgatctattttaaaatccttacaaggtcccattttccatgaggaggttaggaggggaatgagaaagtaagagaacctatctctgcatcactagattgagcttcagatagaatttcaacatcatatagataagataaagcattacgtatatattatattacattgatatcatagaagagcaaaactgcacatgcataagaatttatacacctatattttcgcattgagaatagaggccccaaatgtatttcagtttgcttggcagaaaagagtagaaccacactccttgatgtgaaattgcatattctctttgtgtccctctgctcttcacataccttgactcacacatgaataggagaatagggcaaagcaccatctccctatagcgttcaaacggaatgttctagaaatgagtggagttggccggggcccactgccctctgtggttacgccactgtgggtgcagggtaggtttgggtgtatgccgaacacctactgcccatgtgaaggagagcaaagcaatctgttgatgaagcagtttgttgtcattgttcctattctgatcgttatttgaaacagttgtcttggctatcgatttttgcaatgatgtttttgatgtgtcatttgaatgtgaaacttaaaattaagaatattagagttggaagagatccagaatcatttagagcaaatctactgaatttattgatggggtaattaaaggccagagaggcaaaatgatttgctcaatgtttgtggcatttcaggcagaaccaacactagacgttaacttcttgggtcattgggtcacactttggttctctttgcactctgtgagttatgaggcacctagacaaggaaaggaaagccatttttaggaagatgtgtctgttattatagtaggttaggaaggaagattgaagtttagaagatactgtcgtgtttcagacctgagacagtgaaagaggacctgaatagggtggagtgaatgggaaaaagaaagaaaaggtgaatatgacatatattcaatatatgtcaagataaactcataggacttgtgatgagttaggtaatgcagagcatttgtacttggtagttagaatatatatgttctatataggcaaaaacacatacacacacacaaccctcatacatatacctatcacagatacactctgtattggatattcataacatgcatatcactaataaaaacaaaacaaaacagcagtcaaaacgttattttctcctttaatctttgtaaaaacactgagagatattgttgttccctttacatagaaataaggctcaaagggtttaaatgtttgcctaaagtcataaattagtgtgaggagctgaggtgggacttgaagacaagtttcttaattaaaatctgttctttccacaggcgccctttcactcaccccaagcatgtagggtctgaattttcttctgtggtgaatttttaaacctgttatatcagtctattcctttattttaggcagtgcatagtacggctcctcagaaaacatatatcccacaccctcttatctgcttttccagatttaagttgccttaagctattggtttcctctgtttgacagcatacaccatagtgtttagagatgtagtgcattatggcattaattttgcctggggtctttctcgagctattcatccttcagcagctggaggagagccatatacactctaaatctgtgtactacctagcgaaatctgtaaagccaagcttcccatccaggtcatgattcactgcatcctaggagtttgtgcatagaagcttgactcttcagtatggtgtctgagtaacagatcacattcttcatggatgtgacagttgtggcttctctttgggctttgttaggcataggctagactgttagacatcttacaaatctcctctccccacagtgccttacacagcaccttgtctgtattaattgctcaaaaacattgttgaatgcatggttaatgaaagttattattggcattattctgtggacttttttctttgacatttaacatttaaatatatacagatgcatatatatacacatacattatatatacacacaaatattaaactatacatatgtattctccatacatttcaaatatacataattgataagtcagtacacagataagttgtatgaaatctgaaacatgaagttaaggttttgtgcaacatgagtgctttatattttagacagattagctggggaaatggtagtccaggaactaagcacaactctataaataagggcaaccaaatagatatgagggatctagagataaacattttcagtttagaatactgtatatttcttttttttgacatatatttgaaaagagagaaaacggtaaacccaggagcagtaatttctaaagtaataaagatttacttacatggccttctgttccttttgttgataaggatgaatagaatgatataatgccctgttctctccctctagtttctgggccactgttatattgtctaataatatccactcataggacatagggtggcctttctctctttttataagggtataattatccagtagaaagtcaatattcctttcatcaatttttaaaattacttaatttcttttgtttcgataggtataggccctgcacggccttacaaatgagtaagggatacagcccaggagcaatgtccagctgcactgtttttacttacataaactcatgaacagtgtgtcagccagctttacacactgtgaccgtgagggatgtattttcccaaacattactgatcctttattgaaatggaaaatttagtttgcatgtggaataatttccctttgcaagtcaattgtgatttattaaatttccttgtccttcacaagatgtcaatggtgttatctcttgatacacctcaggacacgtcttgtctgtttgtcgtcactaggccttcatgatatacagcctttatgatatgtcttcctgaactctgaactcttgtaggaaatggattctatcatataaaactcagtatcctccaaatatgcctcagatgaagtagatttcagataaatatttgtaagttaatgagataattttatcctgtatgaaaataattctgaaatggaagacagcatgtagatacatcgtaaatattgttcactctatgtggacatctctccatggcaaaagaagactcagacaaaccaaactattatactaccaccaaaaaagtctgtgcaattaaaaaacagtgcaacacaatgcaacaatgcatccagccatgatggtctgaataggtgcaatctcattaaggcagaacacccccatgactgcttcatcttgcatctgtggtgcctagcatggcagccagtacatggtgggcattaaagatagacatttatgaatgaatgaataagacgccccacatttaaaactcagaaaatatgttttatgctattagttttgcttgaggtcccatttagtatgtgttatgtgtgaaggtgggaatagtggcattgtaaaatggaaggagtacatttttgaggtgattagccatagaggaaaagctgtaggaacctatttgggcaaatgacaccactgtgcaggaaatctctaaggagatctgtaattcttggctttagtacaacaccaaaaaaaaatcatccataccttattatgcagaaatttcccattattaagagatgttaatagtgaatatgcattcatattcactattaagagaatgaacacttattcagtatcccctctgggcagagctgattgataaacatcctggtgttaggcaaagactatagaaagaaggggtaccttgtgtaaaatgtaggttttctctcctgattatgatttttgccattactacaaacaatatgttgcatttgcatacaacttacaattttgaaagcatcgtctcaaacatacattattcttcactccatcttccttgttaggacaagagctgtttttctggtttcatacatgaaagcctaaataatgtctctcacctgggaaaaagtagagacaggactagagcccaagccttccaggcatccattctttctgcctgcccacttagacttaaagtaagactctgacagcaggctcttgaatttacctttgcttatattataacaacatccaaagcagggacaaccttgatgaccttgaccaactttaacacttgtgtaaatacaagaaaaatcaactttgctaaacttgtggtgtgcccataggtgacagtaagctaaaatgctaactccatggcataactgcatttgctacatgcagacagatagagttgatgtttgtcatggtatgagcccagggaatcagacaagtacttctctaaactcttcccactcaacttaatacaaggtaagcaggtcctgcagtagttcaaggacctgctgcacactgtatacagaatagggtactgtttaacagccttttatctgtcatacgtttcttttcacttactactgttactgtagcctgcctccacatcaagcttcgctgcagaaaggagatggatagacacctttaaagaattattcttcccccacttcttccctccatttggcttcctcttagactgtgcctcaagtgaagacaaggctagttacggcctcattatgcaagaggtcagagtgagatgtgaggtgtctacctttctctctaagcagatgcacccaaccaaagtgtgcagaaagaaccactgctgttcctactacacgctgattacagttgcacctggagccacattccagcatcagtgtcattcagatcgtcttagttctatccaagaattcttgtagaacctaagaaggagagctgatcagactaccagcacattaactcaaacatgccacggagtctaaataaatcaagtaatatcatacttgggataagatttaatatactgagtttgtgggcgagccacgatgttttgaatctattccatatgcctaaaatttggtgatgatatgtcttatttagcatgagttctcaagagtagccttagacattgtgcatttccgtgtcttgatatttggacattttcttgtgggtgtcctgaagctcttcctcactttgaggtgacattgaattgcaaagtctagacttttctgaaattgagaattatattatgggatgggattgaaaccatggttttctattgttttgcttaaagctaactttgaacaacaacagctcagctttagaaggtgttttgcctataaaaccattcacataatcaaaagctgaagtgtatgcaggatcagcttaatcaggacagagttatggatagccgtatacttccctagtgtttatagataggattgtgaatttggaagggaaacccagtcagcctaaattacttgtgttataactggagagttcactattctccattataatggggttttgaagatatgatatgcctacaaaactcttttttgtttttaaacttactatcttttttcattagtaaatatgtgaaagttttagtatcagtgctttttgctttgaaagggaatacaattattaattttacccatttcttattatgatattgcaaacagacaacagcagagacaaatacacatgtgtacccacttgctacctatataaggccttataatcattttatattcactatagatcgtaaaaataaacactacagactctgatgagggtctctatactcctctgagatatcgaccccattgctccttgagaaaaacactatcctgaatttagaagttatcattcccataagtgcattcacattatattttagtacatatttatgaattcagaaacaatatataatatgcttttgccagttttaaaaatccacataaatataaaattgtatgcataatgctgtgacatttaaaaatctaagattatattctttatatttattcctattaatacatgtagctctagctcatttattataaccgctgtatagtatttcattgtaattatatggcatagactatgctctcttttagatagacatttaagttatttctaattcttaactcttagaaaccatgttatggaataatttttctggagttgaaacagaaaagaggaatttggggacttagtgcaaggtacttgcttcaccatttattgtgtaatacaagttttaataacagtaaattaaagtattagaatgtgtgcattcaataataacattttgtaaaaatatttcaaagatttcaataaaaattaataaacacaattggtagaatatccctctgcctgctattggctttgctgcctccctagtctttagttccctcaacccagaatacccttggaattgccccttctgacttggctggccctgtaagggcttcctgtcttcctgctgagtgtatgcagctttgagtgaagttctttctgtatctctggcaacagttttgttttctctctctacccaccttgcccactctctgactgcttcccatcccaaaggagagatgtgttttcagatggcacttctcatctctactcagcctttttttggagggtgagtggctagatatgggcgatggggaacacaaagggaaatgctaagacacaaggggttttcatgagtggtaaaatgactggagggtttgcaggaggatcagggaggagaaagagtaaggaaaggggaaggagagagggaacagggaaaaatctaggaggggtggactaagatattatagtttcaaatatcagtatttatgcaaaatacacaaaaatatatgctagtactttaaaatggcacctattaatggaaaattaaaaaccggtgttacagttagtagtagtgtaaacacattttcaactttactagataatgtaatatttctttccaaattggctgtaccaatttacattcccaacagcagtgagagaattacccattttaccacactctagttaacgtttgacctttatttgattttgtaatgtttgccagcctgataggtttgatacaatgtttcattgttgtttaaagttataattcattcactgattattaggactgtagtgaatacttgtccagtgcatttttataacttttataatttttaaaataaattttattacaaaggtagtatagttttataatatcattaaaatcattaaaaacacaagcaagcaaccatcagccgccacataaacatacctcagtttgacatatgtatgtgccaatgaagccgttaccacagtcaagataattaacttattcatcaaccccaaaagtttgctcctgccccttaataatttattcctctctacttattgttacgggatctttggggtgtcaattttctagccagaaacctctgtgaccgtggcgcctttacccaggttcttgtcctgcatccaggaagaatgaggtacacagacaagtgaagagtgaacaagataaagatgagttttatttagtgtcacaacagctcagaggagacccacagtgggtggctcctctctgtaggtatgtcatccgtggagtgttcagttctcagcagagaggaggctctggagagagtagcctgtctctgcagctggttgtccagatggctgcagctctcagcagagagggtagctcctctttctgctggttgtcctgttgtctctttgcccccttcggccatcctctggctgtcctatgccctgctctggctgagcccagggcttttacagacctcagaggggaagagggaggaagtgcctgccaaatggtccatgggtggccattggtggcccagaagaggcaccaagactccccactctggtctgcaggactggcgtcccagcccccagccttcaggccctcactggcctgaaagtggggccttactgaggaccctcctccttctgctcaagaatcagtctgcctcctactgccattcatggccctggtctcggccccaacccctgctctgatcttggagcaggtgccaggagcagagagaggccaggcaacgggagcaaacaccccccccccagcctataaggatgggggtccttcctgggggcaccgtgggtgccagtggcagagacgacccagatcctgcacctgggagggcaggcacagctgcacccagaagctcccaccctgccaacttggaaggggcgggactcctacttgtccccagctcctgccttctccatggagtgggaggcccaggtctgcagctgtgggtcaggtggctgcagctgcacccaggagggcagattctgcctgttcttggcacccctcaagagcacaaggaggcttggatccacagccgcagtttgggcggctacagtggcatccagggagctcccgcccactcagaagggatggggctcccacccgctccatggagtgtgcagccccagcagtgcctccctgctacagccagtgtgatggcatcaactactgccatcactaccaccttcctccctccaatctcttggcaaccatggatcttccttgatagttgctttcagagccttctgtgttgtaagtgtgtatcaatgggtcattccttttattgctgagcagtattcctttgggtggtggtactacagtttatctatccgctcacctattaatggacatttgtgttatttccagctgtttaattgaccacttaatctttcacagaagaatatatatatgcgcctataaattttgtcatatgcaaaatgttaaataggaaataatccctgttagtattgtaatcagtgcctgggacaggacgtgcataggtattcagtaagtaggtgttaaaggaacttagacatgttttggcaaaatctgcaatctaattaggaaaacaaagcaagcacacctaagaaattaaagtgcacagagcagttgacctgaatggaaatcagtaatataataatatccatcttctacccaaaggtcatgacacccccccaacctcgccatcagcacttacttggaagtctcccttctttcagttcaaggcatcctaaattaaatcactcatctttcactgaaaacagtccctccactttcttcctgttttgttcaaatcttttcctaactactgagttcaaaacctgaattatttttgtaatgtccctctgttcacatttgattagtttccaagttctggttattctgcttcctaaatgcccccactttgttgccgctatgactgctttagtttagctctaactatctcttagctcagcacttagtcatctatcttgcctccttgactggctttcccctctctaattcagcctccacatggtcatcacacacttttctgaagtgtaattttgaccatgctgctttgttgcttctacctctccatttgctcctcattgcctgtaggctaaaattcagacttcgtgggatagcactgaagatgctctatgcttttcacgctttctccctgacctctatcatgcagaaatatttaactgcttgcaggaacgtgaacacacgatgccgtttcccatctccatgccattgaataaacctgtccatccatcttctggaatgtttcgccaactttgaccaggagaaaactcagaactcagatttgaagccttcttttatctcctgcatacatgtacatttacagaaaactcagtaaagttaaccactcctcatctatgccaagtgtctacaacactgcccttgcactgttgccagatcttccatgctagactgtgaccacagcaagtggctttttacctttgaatctagtaacaagtacaagagcagttacctaataggtactcggcaaatattttgggtgctgaataaatatgagtaactcaaattatggtgctcaataaatacgaataaatgaacatgtgactgaaataatctgatcatcaagttgtaatatcccagccaacatatgcctcttgtattttacttgttttcacttgtcaccaccaccttagtcaaaaaccatttaactttcttcctgaattgttgaatcagcttgctaacctatgtcagtaaattatccctcttttctaaactatagtaacacagattagctagactaatcttgcaaagactgctttaattatgttatttccccattagattccactgcctggctttcaaaagctctagctactctaataatcagacttttttctattgtataacacagtctccatttcagctaacccattcttcccacttgcccataacaagctctgttccttccagcttgtgtgccggtgctcacatttatccttttgcctgaatacccttgtcctctcttcccatctgtctgaattctactgactatttatgccttgctaaagttggcacctgcatcatatctcctttctatctatatctatatctatctatttatatcacatctccttggaacttctgaactatagcaatttgctattcaaagcgaatagaagttacaatgtacatattttctctctatatatcggaatctatattttcccccctaccctatactagaatataagctccacaaaggcagagaatttttgtctgtattgtctgtaaaggaaagccaagggctgagacacacaatatttcttaaataagtgaatgaaagtatgaaattaacagttgcatggtgctggttcaaataatcctcatttcctcggaagaaaagcactttgaagaaagtatgaacaaatttgaatttctgaattttaaagtatgtgatactttagaggcaatttcttgagtggaattacaaaaccatattcacttatctaaaatgtagcattaaagttctgaattttagaaccacttctgaacgtcatgatttctgtacaagatagtgtcaattagtcaatcatgttatctggaatttttatgcattagtataaattgaaattaactcaacatagttctattacagaagtggataaatggagcatgaaatgatatgagaacattctactcaaagtaaaaaagtagattcaggttcttggatccagattaatgtgatgatatctgtattttgacaggggtaaggccattttgcaagtctttcctaaattccatgattgttaaaaggaaaaaattaaagctcactctattgaagtccttagctcaaaatttgacacgtactagatatgtgttaaatagttgaatggaagccattggagaaccattttttttaagaggagggggacttcagaagggcagtgacatagttccagttgaatttgagaaaaaaattagggtaattgtctagagaatgatatagagagaagacagaagatagtaaaaccaggaagttctttgttcattcatcaaggttcagttataaagcacctactacttggtatatacctagggctgagtgaataactaagattctgttataagtatagcctgctggaaaaaatttagatgaacaattatgatgacaaaatttaagagtgattagagggaaaggagtgaacactttggaatctattctgctagaaatgctgattgtacttggtgaacggtgtaaaaattgagggtttgaagacacactccagtgttctgatttgagagaccaatttaataatatagttagagataagagtatgagaaagtatgaaaggaatgatgatggggttggttgtggacatatgaagttagaagagtctatagaaaatccctgtggagataaccagtatagaattataggtaagttctggtgctcagagagatatgagggctgaggagggatgaatttggaattaatcaacaaaaaatagttgttgaaaacataaatccatgtggaatttccccagaagggtgcttacctcattgagatgaaataagggctgaggctgcagataaggacagcagaatgttaagaggacaagaatgacactcaaagacagcacatcatggctagacgtgacgtcaagaaaccaggaaagagtagttgtatcacttaaaatggtttcaactacaagtaatagagaacctgacccacccacttttaaacaatactgtaatttatctgctctcataactggaattccagaggtagactggcctcaagagtggttgatccaggagctcaataacttcatcaagaaaccagtttcctttgtctccttttgcactgccatccacggctgtgaatttgtcctacatctgtttctcctctggggatagaatgactggtaatacaagtcagactacatgctccctcctttatattcagcggaagaggcagtttgaataggaacctctcctagaagaatgaagaggaacttacccccaagtctccagcaaagtgctccttctgtcccattggcacaaatttggtaaactaattattcccaaaacaattattaatttagattaagaattaggcctccatcttctggggctgagggtagatttagcttctttttttttttttttttttttttttttttttgtgaaatggagtctcactctgttgcccaggctggagggctgtggcatgatctcggctcactgcaacctctacctcccaggttcaagcgattctcctgccacagcctcccgagtagctgggattacaggtgtgcgccaccacacccgactaatttttgtatttttagtagagacggggtttcaccatgtcagtcaggctggtctctaactcctgacctcatgatccacctgcctcagcctcccaaagtgctgggattataggcatgagccactgcacctgtccagatttagcttcttttgaggcaccagcctttgctggtgagaggtgaatacctgaataaaaattaagattctgttaggtaaaaggaagtgagacagttaatgcatagcagacaacaaggagcactacagaagcattagtaagcttaggagggagaaaggttgaggagaagttattaacattgtcatataccaaatgggtcaagggaagaattaaggagaggcggaaaaatgcccatgagagcagtaattagaatgtgaccccagagacacaccatggattctaattaaagtggatagagggtgactgtgaggggaaaagtagttatgaggaattgagtctagtttcaagattctgagttttgaaagaaacgataaagataatagctagaggggggaaataatgagaatcaggggtttcttttttgttttgttttcatgatcatcaattaaatgtttcaaagttagtcagtgatttctaactgggctctgatgaaggggctttgagtgaccagttgtatgaacttagtcaacttgtcaagtctctttgacttctcatccattgaaagggaaaaaaagaaagctgtttttatcctatggagttggtggatttcagaaatatttttcttcagttttcttttattttatttataatttcaacttttagaatcacaagttacatgtgcaggtttgttatatgagtagattgtgtgatgctgaggtttagggtgtgaatgatcccatcacccagattgtgaacacagcacccaacagttagttttcaacccttgcccctccctccttcccacctcgattagtccctagtgtctattgttcccatctttatgtccatgagtactcaatgtttagctcccacttgtaagtgagaacatgcggtatttggttttctgttcctgcagtaaattcacttaggataatgacctccagctggtacatacataccatggaatactatgcagccaaaacataatggaatcatgtccttttcagaaatattttaaagaagagaatattgtttatattttaggtgtgtgtgtttaggagatattttaaatgttatactccatcaagtcttcaatacatttgggggaaacagaaaataaactaattttactttctacctcattgtttggtcttaggctaaaatccatgttttacggagaattcaagaaatttttaaacttcaggtagaactgtgttttttacaaatgtatagaaagcatagtgcctaatgcatggtagaaactcattaaatgattattaaattgttctacttttcatatttaaggatgaaaaatattagagattctccatcaatcagattataaaattctttatactggagaaatttcttataccatattttttatttgtcagagaattgattctaaaaaacaataaaactatgccacaaaaaaaactagtaatattgagtagtgcagcaaaacctgggtaactaagtctaactaggtaggttaattgaagttactggaaaatttattcatacattcaacaagtgttcattgagcacttattatgtgacaggccttgtgctaggcttcttgctctcatagagtatacattctagagggagagccaattaaataaattaatatacaatatgtcagggagataattgccttgaagaagaataaaatgagataatgggatagtaggcaattagtacaaatgttttcattttgtatgaggagagcaggaagggaatctctgaacaggggatgtttgagctgagacacaggacatgaagaatggctgggagaactgtgttcctgggaaaggggaaatcaagtgcagaggtcctcacctgggaacacattgggtgtgtttgacaaacagcaggttagactggggagatgggatgtagggaattggggctctgtgagtgaggggaatcacctaggagatgaggtcaagaaatggggcatcagtgtgccatataaggaagcaaggccttagagactgtgggaggaatttgggaactactctaagagacatgagaaggcattagagggtttagagcagaggactgattgcattgagctggttatcactctaactgccttggggagaataggctgttctagggcaagtgtggcttggggagataaagttcaaacaaatgtaactttttgtttccttgaaatatttaataaagtaaagctacctattcagggagtttgtgcaagtaatagcactgtcagatctactacaataaataggtgataatggtttgtaattagcaaagtaactattcatatagaaatcattcagttgatgctagttgactaatcactcctcatgcaatcttactcttgtatttgatgaactttgttttgaagccagtgcaatatgaagtgcaccatccctcctcagcagggacaaattaccagattattgagatcagtctccttaaaattaatgttattataacattctgttgtaaagaaagaattatatggcaggtatttttgtcaagcttttaggaactgttggagtgctttaaatttataacattgtgtgttccccttaaatgttcatggttggaacacatctaattttatgcaatatttaaaaacttttttggcagtgagattcatagggcagaagttacagaatgatttcattttggaaaataagtgaaaagtaaagataacacatgagcccaataaaatagtccattttcttgatttttttttttttttgccaatatctttttcatgagggcagaccatggtgcattattccacccccgcccccagcatttgcaaaccaccagtcatttccttagggaccttaacaaacggaagcattgaattctcttcagtcagtggtgcctcccacctgggaaaaagacagctctgtaattgaatcccagcatatgcattgtcatccctcctgcctgctctcctggtgctacagcaaattaaaagatctccaagggtcagcctgcctcccagctgccagccccagtagacatgagggagccagcaaggaccaggtggagcagttttgagttgcactaaacacacggcgcaaggagattggccataacttcccaattgaatatttatgccagccacatttctcatcttgctctttggcactgaacacagggctgagctcctgctgatttcagggcgctgcattgtgtttggatcagctttgtgaagtattacaccagatgagaaatgggcccaagttcctatcactaagacggctggttgactttttttttttttttcatggagactatcatgttatagtatcaaaagctggggcagtgagggagttccctgacatctcctgacactgctgttacatgaatcgtacacaacttcaaccattgcttgctatggagtggaactttaatggctcctattcagggacagacaaatatactctgatttgtatattactgcatgttcgtctttttttttcaaggttttatttttgtttgctaaggtcagggacttatcacagggccctctcatcacatttcacattctgatccctcgtggcaggtgctaacttccttgctctcaaactgaatgggttgagtaacctcataaagatacagaatgtcttggcagagaaaggaaaagactacagaatgcctgtctatggaagagtctcagtgttatttagttattagttagccctgtaaacccgagaagatatttggaaacccatctacacacacccattaactttttctaggtggtggatcacagacttaaattcatgttcctgattcctaatccatattgttatctaataaaagcacattccttatgtagattttatcatgaaatttgcttaaaaaaatcattctcttcaattgtatttttgtatagcataataaactatagatcagtgaaccactgaacttcttgtgaattgctggttctaaaatgaaattctcaaatgattaaatttaaataattaggaggcagtgataatgagaagccaatataagttctgtaggcaaaagacatgccagagtaacctcatagtcttttgtgacagcagtaccagaaaggaagagcaaagacatgcattacacttagaacacatagaattcagccaagcgcatgccagaggctcggatatttatgagaaaaagacagaaattagctcaataactattcccgatgaactgatataaatctggaggtatacctctagtaccctctttaagacctgtccttggcattgtgcttttcaacatgactacaggcatgcgccaccacgcccagctaatttttgtatttttagtagagagggggtttcaccatgttggccaggatggtctccatctcttgacctcatgatccacccaccttggcctcccaaaatgctgggattacaggcgtgagccactgtggctggccaacatgtttattgtgagggcattgagggcatgctaagaaagtttgtaggtgagataggaacttcactgggaaagaggacattgaacagcagaatcagaatctaacagacaggctgaaacaatggactcaagtccaacaagatggcatttaggagaaacacatgaaaagctgttcactcgggtcccaaatgccaactgcatgtgtagtgagcgactgggaggacacagggctctcaagcatcacatgggacaaagacttgagaatttcactggctgggtttaggcatgaattaggactgcacagtgtctgaggacaaagctaatgtactcttcagtctacactgacaagtgagagttgtaatagccttgccatattttggaccacaactgtgtgactatggggaccactgttaaagaagaatcctggagatcttatggaagagaatgctaggaaagtaaaagagtttgagatggactgagtggactgagggaattggagatgccctccctgagagagaaaaagctcttgggcaggacaggtcattgatgtgtgaaagactgttatatgaaagaaggtttagacttgacccatgacaataaatgaattttcttggaccaaagtttcagggaagccccggatagaaaactttctgatagtataagttatctgaaaatgaaagggctgcctcttaagaaaatgattttcttgacactacaagttttcaaatagacaaaccaggctgttttattgtctgtgttgtagaagtgattgtagtaataaaatttatgaaggatgcactgtaggccaacctttggatacttttttttgtgtgatcttatttaattcacacattagccttccagaggatattattatctccaatattgagactaaggtcccatgactggtaaatgaggcagagtttaaaattcaggttttagttgacagtagagactcttaacactgctctcatagccttaagcacaatattccattttgcttttgaggacatttaatgtcccttcagaactggaaatctgatcattttaataggttaccaccttttttttgtacattatatcaatgctttgctcttgttagtacaacattgggaaacctagatttgtttatcctgtgaacagtttgatctttactggtagagttttttcttagaaaaaaacagtgatatgtttcttagtacaattgatggcattaaatgttttgattgtggtcttatggctttactccactacttgctagaggaaaataattttctgcttcattaatgatggtttgaactgatctgacagaaagtgaatcctgtgccattcaaataatgcttggaaaatccattggatttttgtttatttctttgttttccgattaagggggacttttaaaaacaaaaatttagggaaatgcttctggttttcggaaacatttattcacatttcttggggttggagagaactgacatgttttaaatttgttagagtcatagtgaacaaaggggaagagttgcaggtctcaagtctagagctgtaggccctggaaaggaaaaattgcaaaagtgtccagagaacagtgtgttcatggctttatttgctgttagtccgaggcctgaggtcaccctggcagatgggtcacttgcactgcaagcactcatgctggcctcagggacagggatgaggtcactggcacatttcaagaacagcttcctttctttatgcatacaccactttcctggtccttgttaagggtctccttcctcttgcggttcccatctctgcagctgaccccagttctgactgtgtctggggaaatgggttggtgagggcctccttctgggactatctgtccacctgatcagtgatatagacactgtgccgcggagaggagccacatctcccagccagcctgccagcagagtaattggctcagggttacaagttgtgacaagtggtttttggatcctaatctgaaatatgtcggtttaacatgacctggcattgttacctgatgtttttaaatgcccgtactttttccagaaagtgggaactgatacacagcagtggcatgtcctactaattttcataagatctaaattgatcaggatgctttattactttttttattccacagtatatttttcctctttgactctaaagtgcatgtgcacgcgtgtacacacacacacacagacacaaacatgtgtgcacacacacacaccccccctcactcatttgggagatctaacactgcacatataaatattgaaacttccaggctgggcgcggtggctcacacctgtaatccaagcactttgggaggctgaggcagttggatcaggaggtcaggagttcgagaccagcctggccaacatggtgaaaccccgtctccactaaaaatacaaaaattagccaggcgtggtggcacgtgcctataatcccagctactcaggaagctgaggcaggagaattgcttgaactcggaaggcggaggttgcagtgagccaagatcgtgccgttgcactccagctctgggcgacagagcaagactccgtcttgaggaaaaaaaaaaaaaaagaaatttccacatgcatttccatcagagaactaattccatctccccttacctagccccttgctctcctgcctagcctgtgggaatctttgaagattctttgtattctaagaactgtcccaagaacaaagtagatgctcaagcaatgtttgctgaactagactacatttgattccatgtaaccaaatgactttatctacatttttttctattccttttattcattagcacccactaaatatactccactattctaggaatagggaaaaaaattcatgaacaaattagaaaacaatcctaccctgatggtgccttacagtcttaattacgtgaatagaagctattgttgttattcttacttttcaggtaaggagaattgaggcttttgtcacataggaagaaaaaattatgagcaagtcttgcccttaaaatcattctgatggtcaaatgcaaatttgctattttggtggactcttgagaatgtgggaaatagacccatgtccactttatgcaaagagaaaaagctggcatgagtgggagtgtttgaacatcaagaatgcttttgcatttcttgggttgtgaacaactgatggagtcaacttataaataagcaggatcatgactcattaccaggatctgtacagaataaataatggcttcagagttcgagtagactctaaactacacagttttaaggcttcccctttcttgggtttgaagaggatatttcaaattattagaaagaaattccatctatgctacagaaaaattgcaggaaaaaaaaatccccagaaactagttgcatgtctttacatttttctttcttttccttttttaagtttttgtgtttatgactaagttcattttttatattgtctggaaagatgctaatattcaaatggcctcatgacataaggaccactaacaaggggttgaaataaacatcctggaaagaaagaatgactacctttaaatattatgagcagtgtacatttggcagagcagatccttatattgactttagtaacctgaaaactattcaaaatgttttatttcagggatatgcattcctgattgtgatttcatggtgtgaataaaataaagtcacttaaattgatagaaacagtctgagctctggcatcagataaacatgagttcaagttctggtgctgctactttctatctgtgtaaccttgggactgctgtgtatcctctctgagcctcaatttcttcatctgtaaaatgtggaaaattatatattaatacgttattttaaagattaaaggtgataatatatatatgaaaaaccaaacacagaatgtgtcatactgtaatagtccattttcatactgctgtaaagaactgcccaagactgggtaatttatagaggaaagaggcttagttgacttacagttccgcatggctggggaggcttcaggaaacttataattatggcaaaaggtgaccgggaagcaaggcaccttcttcccaaggtagcagggaggagaatgaacacaggaggaactaccaaacacttatgacaccatcagatcttgtgagaactcactcactatcttgagaatagcatgggggagaccgcccctatgacttgattacctccaccttgtctctcccttgacatgtggggattatgaggattacagggattacaattcaagatgagatttgggtggggacacaaagcctaaaccatatcacatacaatatatacacagtaaaaactgatacccattaatcatttcctgttgcttaaacctaaggtaaaatttatagattttacattctgtataaattggaattaaaaagaaaatgaagtgtcacaattaaattaatcacaaaacatataaaaggaaatacaggaatatttttgctgtattcttagtagccatagtgtatctctgttctctgaagacttttagttgcaagaaacattgacctacctaaatgaattcaagcataagtaactaaattgtaaatatctagataattgcatttcggtttcctgtggactggaatgtagaatgccaaggctatttttattttgaggtttttgtttttcaggggtaacattttctgtttcttctctgctccgttctatggtgtgtgcttcatccttttattcctgaccggtgttccttctttactcatcttgtatgtcgtccagattggctatgagcccctacactactttgtggcttcagtgatatcattaagcagatacctcccccaggatcccttattcgaattcatcaaaacccagctaagaatttctgatgacattttctagaatctttaagtttacattaatttctatctgttccactttatggacgcttactgtactttaattttctattcacaggacgctgctttgtcatttagatggtaaactccttgagggtagggccatgttttcttcactcagcaatcctaacacctgtgacagtgctttacatgtagtaagcaaccattaatttgtttgatcatttaaattaaaagtcttggccgggtgcggtggctcacgcctgtaatcccagcactttgggaggccgaggtgggtggatcacaaggtcaggagatcgagatcatcctggctaacacagtgaaatcccgtctctactaaaaatacaaaaaattagccgggcgtggtggcagccacctgtagtcccagctactcgggaggctgaggcaggagaatggtgtgaacccaggagacagagcttgcagtgagccaagatggcaccactgcactactgtactccagcctgggtgacaaagcgagactccgtctcaaaaaaaaaaaaaaaaaaaaaaaaaaagtctttgggttgggcgagtctagtagaagataatgactttacgagcatttagcattgactgtaattaagtctgttatcgggatgtgctacagatactacttggtactgtttttgagctcatctttgttcttggatttcacatctctttttcttctgtttgttccatgattggcaaatcttctgattcctgactgccatttaaactcatttatcccagtttttcacacttaaattcaccttgtcacactcacatgcttgatttcacagctgtttctaagatggttatggcttacacaccatttcactcattcatttattactgaaatgtttcttgagaaactgcttttcttgagaaactactttttcaggcagctttgcaggcaataaggatatagcagtgattcactggaatgaaataatacaaattaaaatcagcaaaggaaaaaggcatatggggcagagttcaggagaaaccggagtttatttccagttgtcttctgtccagagttgtacaaatagcacttatttgtccctacaacaatgtgcgacagtacatgcagagcgttgccaatggtggatgctcatccaagccttagtgcccaggggtcactattattggaggtcagttatataaacatgtctgaccacccatgtggctgaccttcattactgtttttagccccttcataagtcagactgattctgcgtggctcaaagcccccaccaggaatcacattgttagcatcattgttagcaaagagtatctggtgtggcccaaggctccagctaaacacaggtactctaataagttaagatattccaagggcttaaaggttatctcccaggaattggtcaagggacagacctttcttttgaatgtgcagggtttggacgatacactttttactgcacaagggctaacctcacttattttctttcttctctcaaggaccaagccctgcgccatctaaacaattgtttccaacgttgtttcctcagttttagttttatagtggcaatattaggtcccagttactctatcatagacaggaattatagtcaagtaatttgaacaactctttataaaatgttatttcattcttattacttcctttctatgcttttgaatatattctataatgtgcctatataaaaatgtacataattaattagcacatataaatgacacctaaataaatatgtacacatatttgaggtgcttactttttttattcaattaacaatctagtagtttggaagctactgagtttgacagctgtttatttgcatctttaataggaagggcacttcaggagttaacaaagataaatgaaaaagaatcttgttatcacagacatcctgtggcagcttcttttttttcatttgttttctagaagaacgtgtacacaaatgacaataagttaagacagagtgtgaaaagtgctgcagaggaaggtaaaaataatataacaaaaatatgacagtatgaccaggtcacttctggttttgaggaccagaaaaagctgtttggagaaagtagcatttctgaggtatttaaacattgattaagatggttttcaggagctatcagacaagaaagatgctgtacaagctaaaattgtgaaggggaaattgtgacaatagtacaataatcatttctgatacaagaaactgtatactaggtgattacttgaagataaagctgaatggagagttgtgaatgtctgggtaaggcattgggacaggatagtactgattatatttgtaaagagaagacatataataagagctaagtttggatttctcagaaatcacatataactttatttttgttcccctctatgcatttcttatgattggctttaataaagcattctgttattttcagaatttgactgatgtgtttagctgtggtttttctttttcctatttgatgtttgtggtgctgtaaggctctggggttgatgtttttcatcagatttgggaaaattttgaccattacttattccaatattattctgtatcattctctttctcctgtttttttgataatattacacaggttacttatactcaattaagtttccactcccacttttttcctctgtgcttcagtttggataatttgcattggcccagcttcagatatcctggtcctttcttttgcaatgtccaatatgctgttaagtccactcaaataatacttatgtaatatattgttattttcattctagaaattttatttagttcttttttaagagtttccatttcttctccctatgcaatatagagatacatactgttataaataaagcttcaatgctgcaaaagaaatggcactcgaatataaaattttctttttaattctcagcaaggcaacatacttctatagaagggtgcgcacttacagatagagcaatggtgagtgcacacctggacaacagagggaaaggggttcttacccctgaggcacatagcccctgctgccgtgtctttcccctattggctatggtgagatggctaaactaattctgattggctaatttaaagagagtgacagggtgagtggtctggtggaaaaaaatggttatccagggtggagaatgagtcagggcggagcaggtagtaggtaattggaatgagttgggatgaagcaggtaatcagaatgagccagggtgaagcaggtaattggaatgagtcagggtggagcaggtaatcagaatgagtcagggtggaacaggtaattgaaaagggttgctttatgaggaagttaagtttaaaagtagaaggcaaagaatcaatactgacatattgattctttgaaaagaaatttagaactcatatctaacaatacatatatttttatatatatgttatatacatatatgtattgatccatatatgacagatgtatatatggcagatatgtatatattaatacctatatatggcatataaatatttatataccttttgtgtgtatcttttcctataagttacttaatatattcacattagttttaaaatccttatctgctagtttcaacacttgggtcataaacaggtctgcttttatttactgtgttttctgtccactaggagtcatatttttctgctttttgttaggtcacaaaatatttataatgttctgaacattatatacaataaatcaatagcgattgaagcatagtatttcttttgtgttttatttccccaaaaagtggtagttctttcctctggcagttaaggtgaggatctgttcactgagacccttcctagagttgagcttaactggagctagactatgattagcccaacttccatccctctgaaaaatcactgcttttatatttcaacatttgaactggagaggcattgactgaagttataattttttagggttttttttagttaatatttttatttagtactggcaaggtccttatacatgtaactttggtaaattcactcattagttctagtagtttgggggtttggggtatgaatttcccagaattttctctataaacaattaaatcatttgtgaattaaagttttttctttctaaactttgcttattttatttatctttgtcttgtactggctaagattgctagtagaatattgtggtgaagtaggaaaagcagatgtctttggcttgctaccgaaataattcattatttcatcaccgaatatgatataagctatagacttttcaagagtgcttttatcagacagaggaaaaatacttcttttcttagtttgccaagaatttgtatcatgaatggatgatgaatattgtcaaattttttttgcactcatggaaataatattatgttaatatattgaattacattgatttccacatgtgaaatcagctttacattactggcataaagcgcacttggtcatgatttttttctattgctgggtttgatttgagaatattttgttaaatattttctcatctatgtttataagaaatatatatccatattttcttttcttaaaattgttcctttctggttttggggtcagggtaattctggtctcctaccatggaatgaaaagtgtttccttctattttttctgaaagaatttattaaaaaaattgttactaaaaaaaatttttttttaaataacagtagcaaaaaaagtttgctagtgtttattaattgaaccatatggccctggaggttttttgataaatatttaattataaattaagttttttaatcaatgcaggactatttagattttctatgttaattggatcaattttaataaatgtgtctttcaggaaattcactgattttgtgtgtctttgaattaattggcataatatagtttataatacttccttattaccctttcaaaatcattggtatctctcatgatcatcttttggatttgtaaattttctctattgttcacagattttgtttcaattatttctactctttattgtttccttctacttaccttttgtttaatttccttttcttcctctagtttcttaaagtggaaacttaggtcacagattttaaatccttcttcttttctaatataaccatttaaaggtataaacttctcattaatcactactttatttacataccacagtttgatttgatcttttcattatcattttgttctaaatatttattatatttacttgtgattttttttcctttggttcacaaattatttggaagtgtagtgtttaattttcaaatgttggggattttttatatgtcttattaccattgctttctaaattaattcattgtggtcagatactataccctgtgaaatttcaactttaaaatctatcaatatttactttatagcccacctatatcccaggtctgtcctcttgatagatgttccatgtgcacttgaaaatagtgtgcattttataattattgggtattagtgtcctggaactgtcaattaggtcacattggtgataacgctttccaaatcttacgtgtatattttttaaaatttatttttgtctacttttcttctgttggttactgtgaaaactgtgtcataaaagctccaactatgatcatagacttatctgtttcttcctttaattctgtcagtttttgcttcatgtattctgtatctctgcttgtaggcacatgttatgtcttcttgaataatttattgtttttcatcattttccaattcctttctttatatttgctgatagtcttcatcttgaggcctattttgtgtgatgtaatattagcttgaccatcttttttatgttaattatctgcgtggtatatcattttccattctttgaccctccacctatttgggccttatcacttttcaggttcccaggatgcagatgctgagatatttcctgcaggagtttatttgggttgtgctgtcagtattagttccttttgaggggcatgaaggaagcagggctggattcagagacgttgatctgtagcacagttgcagcaaaggtctcagctagtcttatgagaggccctagactatggtgacccttagggtaagagggaatagtcattttacccctccattgacctgtcagtagatgagggctgccctcagaaagagtgtgctcttgggcacagtgaccccgtttagttgagggcaagttttggaaaggaactcagtagaaagctaccagttggtaacactctcagcagctatgggaatgagttcttcaggatgtctgggcagcgtactaaagtagcacagtatgctgatatttaaagtgcttctctcatagatcccccgtaattaggtcttggttgtataatctaacctgaccatagtcagctttttattggagtgtttagtccattgatatttagtgtaatcgtcaatgtgtttgagttcgagtgtgccatcttgctttgtattttccatttttcacatatattctttgtttgcttccctttttttggcttcttggggttaattgaattctttaaaatgctgctttaatcgttctgttgggtttttgttatacctgatggtatttttcttaacatttaatgctagagattactccatgtaccctaaacttaacctcttatccaagtttaatataacacttcatgccaaatgaaagaaccttgaaaaatataatttcaccccactttttgctgttgttgtcacatattatataagtccctgtatacagtgcaattatttttgcttcaataggctgtctttcatgaattaagagaaaaataaaagataatcttttatatttacctatatatttacatttatgttattcttaattctttcctgtaggtctgagttttcctctggtatcttttctttgagctgaaggtcttcctttagtatttgttatagttcaggttgttggtgagaaattctctcaacttttattgatctttatgtttctttcttccatgaaagcatgtgtagcttctttgggctactgcacagttctttttcctagtttcctttcctttttcttttttttttttttcttttgaaacagagtctcgctctgtcgctccggctggagtgcagtggcgcaatctccactcactgcaagctccgcatcccaggttcacaccactctcctgcctcagcctccggagtagctgggactacaggcgcccgccaccaagcccggctgattttttttgtatttgtagtagagacggggtttcaccgtgttagccaggatggtctcgatctcctgacctcgtgatccactcgcctcggcctcccgaagtgctgggattacaggcgtgagccaccgcgcccggcctctagtttccttttcaaaagatggtcagcccttcaagaatcccaattttattttatcccagttgtagcacaatctctggagcaaacttaagtcaaagtcctattcctgaatgcatactaaaagtgttacaaaaccagctgtcaggacttgggctcattttttgactccctgggtcaacaaggtcagctggaagcttctgcttgatttggagttcacactttatttctagctactgatttcttccatattggtggcaggggatgccaaatttaggtaattaaacattttcagctctctagtgtgtagctaaggtgatcagtttaggagggtgggtatgcatatcagcttaaattgccaagatgacagaatctgcattttttcttacagtattatatggaatagtcataggattattttttgttatttttatatgtcagtgataacatgtttatttttgtgttagtaataacatgttttccatattatgttataatatgttataataacgtgttatgttattacaaaccggttatgaggcttaacttactagggaagcttttcttatttatttatgcatttgtttgtatgtttgtttctcatgaactggcatgttcatctggttgactaagtcttagatactggttttaattattactgtgagatcacgtggtcaatagaataatatttgtaatctattaaaaaataactgctgcaatttcaacttttctctgctgcttactgctctaccaggtctatggaaatgcaggacctagcaagtcctcatactcttgttggaggtggtgatactccaggtagctccaaactggaaaaatctaatctcagcagcacatcagttactacaaatgggacaggaggtaagtgtactaccctgaagatacccagaatcatatttcaatgtttgctttaattttacttcttagagatctgctaataaataaatatcttattatcaataacacaaatcagcattatatccaaggtatatataattaatattgattgttttgatgattttcttgaaatttttgctcctcctgtatagctgggaacattcacagagaatagatagcattgagcaacatttcaagggccttgaacaaggctttctttttgtatttgcccttgtaaatattccacaaactttagaaattgaggtcaaaggatttccaccctggtattagcaaaaatgtctgtatgtgtgtgtgtacacacttactactttttggttttattttgaattataaacatttttcttaaaaatgtttcttcttaaaagtgcttttttaaaaaaacacatggttttggaaatacaaaaatactcatatctgctgcattttcttttcaaagagaataacacttaacatttatttaaaaaggaattgcatttttttcagataatttctcattctattagcccataattacgccacagcatgacaacaataatagctattacttcagatattctgctttattatttttataaaatgtattcaacttgtcagaaaatatggtccaatttttatatcagaatgtgtgtttgtagagattatgtatacacttatatatagactgtattttaaattattcacaatttgaaattttgtagatccatctagccaataaatttagaattatatgacatttaatttagatattagcatttgccacatcatattttcttttaaattttaaataaatatctgtgaaattgatacacctcttcccatttattttagaataacgatgcaatctaaagttttctctgtaaggttgtgtgcattgcaacttacctgtatttaataatgttttacaccagttcccttgtagattaacattttccttgtttatgatgtttatttgtctctaaacatgcatctgtaatttcagctaattaattatgcaattgcaaatgtatagaatatttagtgatgaaaactagagtaaggtacatttaaaccttaaattgggtacaaataataaaatttaaaaactcaagcactctaggaacagactggtgaagttcactaatcatcatatgtatatatttgactgaactaaaacagacctgataaactaaaatgttgaagtcatacagtgcagttataattttggctaaaaggtcagaaaccagtgcaagcattgaagattattcatatccactcagcaggcattcagacaatgaacttttatactgttcctgttctcaggggaaaacatgactgttttaaacacagcagactggttgctgagttgcaacaccccctcttctgcaacaagtatgagagatgctggtacactgcatgtgtttgggtgtgtggatttgcccctctggattgctgtctgttgctatgtctatgtttgcatctcttttctgtgttcagtacttaaaaaggaaaaatgaacatgccttttagttgtttattgacagcagttggttatgatttcttatcaatctgtattcttagatttttttaggtcagtagtattagtattcttttcttaaatttttcataatttcacctaaatgatgcatatattggttttctaactatttatcagtttattacatgattaattttgcccaacaactgccatgtcaaacagcaattgaagcagaatggccacgcatgcaaacttaatcaccttcataacccacagactttcataatttaaattaaattcttaattaaggaaagatatttgctactctacagactttgagaaatattccaaagtatataaggtaacattaataatcaatatcccctcaccttaaaaattatcagtaatgaaagctatacatttaaaactagaaattaatattggcttttaaatttttttgaactatggatattaactgcattcatacctacacacatataaacaaactttctcctaaatcaaattctaatatcctaaaaaaaatcaattagaacaaagccatcacatttttgtggcaccacccagttgtagaatcaaaaaggagctcatgcctattggaaatgtgcatcacaaacaagcagttagaataggcacagtttgcctggaccttgctctatctctgatgaaattagaatatatgatcagtgaagaaaaactacttcaatatgctcattatttgtatttgtatagttttagttttaatgtttttcaattttatacttaaatatacttttcatacttaaaattatgttgctagaatttagatactcttaattctatagcagggtctttttctctaaagggccagatagtaagtatttttggctttgtaggccacatgtctgtgtttcagctgctcagctctgccatcatagtgcaaaagcaacatagaaaatatgtaaatgaataagcatggctatggtccaataaaacttcatgggccctaaaatttaaatttcatataattttcatgtgtcatgaaatattgtaattttgacaactttttaaacatttaatatgtaaaaaaatgttcttagcttactggctgtggtctgttctcaaatagtgtttaacacaagcaacattataatagtgaatatgtgcaataaatagtaatttacctctgatctcaaccagaaacatttttctaagtggtaagcatcaggctttcctggttcttaatgtcagtgttaaaactcgtgagtccatgggagaagaacaaggagttgtgcgtcagggccactgtggatctggatcttagcactacacatactggctgtgtcaccttgaacaagttacttaagctctctgagcttcaattcctttctataaaaggatgataataatacttcttggcaagacttttgtcttagataatgtatataaattgcttaacaataaatatcagtatgtcttcctttctttaattagtaatgcaataataggcaacagctttttaaggaaaatgaaaatatgcattgtgttaacagactccatttggtcagataaatttagttgcatttagaaatgtgtaaactgggctaaagaaagcaaacttttggtaacaaaatgttagataaactttgattattaataatgtagtcctggtagcatattatatgaaacttttaatgaatttcaatgacttttttttttttgaaacggagtcttgctctgtcacccaggctggagtgcagtggcgcgatctccgctcactgcaagctccgcctcccgggttcatgccattctcctgcctcagcctcccgagtagctgggactacaggcacccgccacgacacccagctaattttttgtatttttagtagagatggggtttcaccgtgttagccagggcggtctcgatctcctgacttcgtgatcggcccgcctcagcctcccgaagtgctgggattacaggcctgaggcaccgtgcccggccaaatttcaatgactttttatggtgggtgttggtgacacagactaagcatttgttgcatgatctcttctgttaagaataaactattttattttcaattcttggtttttattgttggaattgtatcacttcataaaattataacaaaacaaaaggcaaaagaaaagaactaatttaatagcttttcaagatcattccaataactcgagatgttcaaaaagattctttatttatttatttaagatggagtctcactctgtctaccaggctggagtgcagtggtgccatctcggctcaccgcaacttccacctcccaggttcaagtgattctcctgcctcagtctcctgagtagctgggactacagacacacgccaccacgcccatcaggctaatttttgtactttacttagtagaggctgggtttcaccacgttggccaggctggtcctgaactcctcacctcaggtgatctgactacctaggcctcccaaagtgtcgggattacaggcgtgggccactacacccagcctaattttttttaattgatataagtagatagtatcacctttagattttatcctactacattggtgctctttaagtatgttgcaatattcttgaaagtaatttaatgtgtttcactaccttaatcctaattatataattacatctgcagatttatatggaacatagattttcctttgaaattgttagcacttgtaaaattattaaagtgtaaaatatagagatagcataaatgcaagcataccttagcaaataaagcagaggtcaggccttgtgggctgtctgctctctgttgtcgctatttagctctgctattgttgcacaaaagcagccatatacaatacctacacaaattaacgtgattgtatttaaataatttgacttatgaacactgaaatttgaatttcattaaaatgttcatgtcacaaaatattctccttttgatatttttcagccacttaaaaaggtaaaaaccattcttagcttaaaaccatacagaaacaggccatgggtcagatttggcccaactcctgatatagatctatatgtgtatacataagttatgttgtacatataagaatgcatgccaattatatgaaaatttacttccagatataggtaattccagattctaatttgctgaatggtgtcaggattcatgatgaactacacttcatatacaaaaatacttagatctattgggagggctagggcagtatctaaaagagtaaattttcactttctcacttaattgaaatgtatcaagttcctattatatgccaagttctatgccaggaaatgggaacccagtgacctaaagaacacaataccagccatccaggaaccaaaagtctagcagagggaggcagacatgaaaataatgaatgccataatatgttcaaggtggatgaacagtggagggagggaatatttgattctacatggagattaagaaacaacttcaaagaaaaggtttgggggaatagaaatcagacattctgatcagctttagtgtttttttctatatttgaaatcgatatagaaaagaatcttctagaatttttactgtagagatgattgactcacatataatgaattagtatttgtgcaatagagctacctactgctgtgggtcagctcttttgtagatttttttttagaataaaagtcataaatggcttcatcaaattattttacaaaattaaatactttatgggtagttaggaatgaaagacacttggtaatcataggtttttgaatgaatatattgatccaaacaagcataaatcataggctatactgattattaaaaccagtaggcaatacttttactgcttccctgcagtgttataaattgtaactttcctgatctaactctttctgaaattctttgtagaaactaagctgtcaaaagcctctatatgtgatttttgtggttcagaaagaagtgtatgtggaattacatatatgtagttttttaaatggacaccttaaactggtgcacacacaccttaatctatgtttaaactacactgatttatgaagatgaaatgtaaatgtaaattatgtgggttacctttcgaggatgaagcctgaattgcaacatatgaatatgaactttgagttatgataataaaacagactttctctaaaagtaattaatcgacattaatgttatacttaacttctaaagacaatttaaggggacaagagtaagactgctttaaaaataagtgtatggcagtagggatgaaggttgggatgtgaagacaggaaatctgtgatttagtagccattactggtacctgacaataattatgtatgaccttgatttagtctatagataagaattatttttaaagctaaaaaaatggaaaagccaatcatgtatgtttatttatttttaccatttgactctatcagaaacatccaaattgcaagtagctaactagtggggagggggacctgacagtacaaggatttcttaacggttctcgacaaagtggtatagaaagtaattttatgccacttttagcaaaacgagctctgactcacttactttgaagatacagaataataatcccacaaaactcttcaacctaggacattaaaatattgtttcttttgaaatcaaaacatttggtgtgtaagcttgcatttggttgagaaatacggtgcttgtcttctataaaaggattttgcttccattttattgcatgtaatgattttggtttgctgtcctgaaaattaaaagccccatttaaacttcatgttaatttaagaaagacaaaatatagcttaacatacatttgaacagtcaaggaataggcactgataattatttggaataaggatttttacaagttagtagtaataagactagatttgtcttactaatgaacatttttccaatagtgtcttgcatatatttaatgaaagtttcctgccactagaaaagtatatagtaggtgtaatttaaagtgcagaaacaaaagcaagataaaagaaaaaagaatacatttcaaatatatttctagcttcagtgaaaataacgttgttttccaagctttatctataattattatataatgtttactttatactagttaactgttatataaatatcaagataatacttcttcgtattctaacatgtcttaataaatcagctaataatataaggagtgtctattcacttctctatttgagctttctactgctgaaaaaatagttgtttattattacctaatattactgctttttatttagaaaaatcaaaccaaaataactttgggacttacttagagatcaagaggtgcttgttgatgctcacttagtatgaatgcagcttattttctgtgacagaataaatgttttatttctacttttccattttgttctacccttgcataggaaaaacaattttacattaacatgtaccacttcccctcaaatgttagcatccattactttaaaattcaggcattttagtaaagctaacacatcttgcttgttactcctctcttcatatactttcccagggatggcttttctcagtaaaatgaacttcctttgtctatctttgactaaaagcaaacctttaaaattttaatgagagatgtttccagtataatgaattagaccaaaaactcactctctccttcttctttctccttttgacctccccaaactttcaaattgaagagattataatcaaaatcaatactaagcttttgaaattgggtctcaaagttgaaattcaaagtaacaaaactaatcttcatgtctggcttctagaccagtttcgttagacacagctcacattctgccacctccattcgcaagtaaaatctatattacaattagtataataaagctacttttgcaaatattgttcgtagatatagatatgcataaataggaatttagctgcaagtagaagtcagaagctctgataaactgtttcttagttttgctgaaggctttgggccatgactttttgtcataaatgttagaactcaattgtaaacattgcgtgaaatggagtagccttgatacccatctagattatactctgcattatcttcatacagacaaaaggaattcttgattagctagtgctgcctgagaggcagtatggtttaagagtttggattctgaacacacactacttggtctcaaattctagctcttacaagctttgtgactggggacgttatttaacctttttgtgcctcagtttccttatctgaaaaataaagatgatgatattatctactgactgttgagaggagtaaatcagtaggtgtaaaggacaagagaagtgcctggaatgtatcaagttcattttaagggtgtgctgttactgttacctaatttcaaatttgctacttcttgatgtcttttaaaatatcccatgaggaagaaatgggaaacatgccttagtatcctattccagtattttgcagtttttattttcagtgaagtatttttaattattctatctaggagataattgcattctcatcatgctttgggcctttgagaagagacactgtcatgagcataagctattttcgtacctgaaggcagcctgatacggtggaaagatcctgcatactagagctaaaaacctgagttcaaatcttagctctaccgtctcctgcactggaacttatgcaggcttttcagcccccctgaacttcagtttccttgtctataaaatggttatagtgatttgtattaattagagttgttttaaggattagctaaatgtacatcatctagcacgtagcaagctctcaataagtattagttttatagtagtaaatataaaggtgataaaatgattttataatgataggtttagttattgataaggtataaccataatagggattactgtgcaaattactactgaaaagtaataaccaccaattcattatttatcattatataaataattagactactcttcagatcattgttgctcttgaaagataagtcaaacttgagtttgcttttttgctaagaaaagtaatccaagtttctttactttctgtttccaacatttacatttctcctctgaatcttccagctgatcatgtcattaataaataatggatactaaattaacccagagctcactgaaggtctggttaattctgttcatgtggaaaagagcataatcattaccgcatggttttctatcaattaatttcttttctaacagaataacattactgacttgtgtgattggtgggtaccctatgactgactgttcttttttgtcctttggctagtcttcagctttcttctttataattttctgcttgcttctaagttatttaactgtatcattcatttcacttatgaccaggaaacacaagaggtgttaagtagaaaatagcttagtttatttccttgctgctatgatatatgtagtaaagccttgccaacaaagggaaaatatggtgtgaccaaatgggtaaaactttattctcacgccccatcacccttgttgcatctaataatgataataatgtgtactaggccactttgttaccaagcaaagattttgccaaaatacaccattaccacgtagaaataacatattaaaatgagttttcaaaaggtgtacttgctgctgtagaagtcatctctttttctacgcttatctctaaacatattaaaaattgttatgccattaaagtatgaagcccaacaccttatagctcgtgagtcttttttagagcttcattgagtgcttcatttttcattcattgtgtgaatgagcccatggtttctatacttttatatgtctccattgtattattatgcaaacttaaaaaatatcttcctgggaaaaataaaatataagctagtaaatttctttggtataattttaattaaatgtgctgaagactgatgatgattccttctttcctattgactatatgagagtgtgtttaggtgaattaaaaatacaaatatgtgtactgctgtttcactttttaaggctgactgcctttaaaagctggatttgagagaactagcttggccagaggattttagattaaatcctctaaatgcatgcccgtttagaagctaaattaacaaactctcttcatcaacttctcaggttttctcttgcatgcccatggtagctagaaatatcactagtagaacggacagagtctttgacataaatttagaagtaaaactcacatgtacttattcttctacgtagtgtctcttcttgcagtcaaaacagagcccttgaacagcagtgaaaccacagccacgactggagatggagcgcttgacacttttactgggtcaggtaaagcctttagctcgtgtgtccttacgtacacatgggggtgcatccacatcctcctcctcgggaataagcaacataagcatccaagctcttagaactttgatccttataaagttagaaacaaattcttattttttgagaactcatagtcatagcagtatgcattgttataaattatatttagaataatttcgcttgatattttagttgacagcaaaagtcccatgactttcttattatggataagtaaacttaacttccttcaaaattattttatacgatgtttaattaatttaataattaagttgtagtagctgaccttgcttcttcaacttacccatttgaattacataaaaactgccttctgtttaacattcgagatgtaaaactatccatttgattccttctaaataaccttgtttcctctctgggtttaaaaaactcctcctgtgtaagcagctgagtgacaagtattaaaggttaatgggctgcaggagagcacatgctggttgcattagaacccagcctctgagagcgctcgaatgcttcaaagagaacaatagcatgtctttaaacatcaatccctttaaaatataaatacctgataactcccagctgggcacaaagggcatgaattaagtagacttaattaaatttttttaagttacaatatttcttcggctttttatctggtgaaaggatttttctaacctgaggaggatcgggtacatcagaaagaagaacattaaacctaggaaagtatttgtggtaattgaatctcggttctgttattctactttaatacattttacatttatccatgtgctcatgttataaggatttttttgtcagtatgttttgtgtaaaaatagttgagatatgtcttagagtattatgtcattatagcatatatttgatttttacagatgatctaatttcactagttcacaccgattagaagcagttgccatatatttacataaatattagtactccaagaaagaaataatagtacataaactttgcacagagactcatatgtactaatccacatcttttctagcagtttacctgcctctcaggagatagttaccagttcagtattctgtattgcacacttaccatagcttttgtaaactttctgcaaaataaatctatatgaacaaattgttaaggaaaggaatggagttattaacttattgctgacctacttcagtagtatactacaagttaattgaaagctgaatgacaaaactataaagtgttagctccagaaggaatttcagagctctctgatccgttttgtagacaagagaagttaaaagtggttaaggccatacagtcaaggcctcacagctggtcagtggtataaacaaatctaaaaccatgtccttatgatcagtccctgggtcctttctgtaatgccccatcagaaaatagatggaaaaagaaataggcatgaacaatcatcctgtctgaaattcactcatattgtctttgtagatattttcgcaaatttttaaagaagtatctagggacgtgctgtccagtaaggtagccaccagtcatatgtggttattaaaatttgataaaattaaaaattttgttttttattttcattttcacagcaatattgcagtaagtatatttttattaattgattttgaaaatcatcaagtacgactagaggcgttaagttctcaatattttcagcaaccaaaatttaactgaattttcaagtttaaacacatttcaagtgtttaacagctgcatgtggttagcagctaccatattggacagctcagatacagaacatttccaacactgtggaaagttttattccacagcagtgatccagggcagggattatcaacatcagcactgttaaagacttgggctaattctttagtgtgggaaagctatcctctgcactgtaagatgtttggctgcatctatcccagaatgccagcagcactcccccagatgcttctttactaaagaagcaaccaaagatgtcttactaaagacaaccaaaaatgtatccagactgtttaagtgtcccctggcaggcaaaattctcacccccacccacttctcatcccattgagagtcaccaatctaagatggcacgataatgagaatgaaaccttaaaattgctttgacaccatcagagaaactgacaatagatattaaacatgtatttaaattaattttcagggcattttgaaaacaagcagttttgcttactgttgtatgcagaaatgatggaatgtaatgagtgttttgggtaatgtttatgattaatcagatgacagtggggaataaaacgaaattctttcaagtttgtacaagatattccagaaaccattctggaaatgaggctgccctttgtgggagcagtctttactttgatctgcaaaatttccactcactttaatccaattttgttgttcttttcagttatctgcaaatttctgaattagaacaaaatgtatatattttagtaaagaagcataaactttcctacgtgaacatcgtagttttctgagccaaaggttttctgttctatttagaaaatgaacatatagacgttaatagaaatttgtaaaggttagttgaggctttatagtgttgtgagtacattattctaatgatgtttagatttatcattagaagaaacatcctatatattcagaattgagttcatgtatatgagttttttctgaaatgtgtaattttggtctatgtcttatttataaaaggttactataatgaatatttatcttcatgattcagcgtctctttggaaaagccttctatttcacaatattgataataactgatgagtatagcccaagtcttgagtgtaccaaggagaaattctgtaaagaagagacgaaagaatatttaatcaagcgtcatttaactatatttctgttagagtatttggtattgttaatcgataaattagaatgaatatctttggaataacactactattatgaagtgcacatttaatgtccattgtgtagtagaacaaagagtctgttttcattaagttgatttgaaaatttaaaacgttctttaagttttgataaatatgtagaaaagtaatagtttattggtcaatttattttcaacgatacgtatcaaattcagttaaattctggttgctgaaaatatttagaagtaacacctttagtcaaatttgatgattttcaaaattgactgataatattcttaaaaatatacttacttcattatcgctatgaaaatgaaaatattctttccatattcagagcttataggcaaaatagcattttaaacacattaaatgatgcatgtttgtgaagcagatcacaagggttcttgtgatatatatagaaatatatcatgtttcataaaatagttagcaacctaaacctatttgtcacttttacaattctaaaagtttcgctccagaattattatgaacaactcagaaaggacatagtactttgaagtataactatgattgattagactaaactaattccagcactgcattgtgtactactttaatctttttgaaaagtgaatgaacagcagatggcctaataaagacactctgtgtgctcgctcagtgtggattggctctaagggaactggctcacactttagagtccatatatttgcaaaacatctgtcttagtcaaggcagatagttaaaggaaagagaaacttccaagctattggatatattccagtttggggctccatgtttggatgaactgttaataggacctcactttcacctttaagttcttctcagaaaagtattatctctaatagttcaaagtatatttgtaaatgtaattgctactgcattttgtgattatgttttaaagaaatcacctgcactttctttaattattactggaatattttagactgcattttttaattgttcatctgatgggtaaaatgagaggaaatagaataaaatagtattttaccattagaaaatgaagtttggaacatcacagtcttattttgtattgtatttgaattagaacagatatggaggaagagcaatatgtatatattgccatcatatttggtattttaaaatgtaaagtttgcaaattttaaaaatttacttttatattttgaaagcttaattaccctataatcaaagatatttattcatagcatattaatatataagagcatgctaatgatttgagaaactggaagaatgacagagggaatgatttgtgagacaaatgtttatttcatgctttttgcacaaaactcaaagcagtttaattaggagaaagtgtttaggcagctgtttccttttagaaaatgaatttctggaaacatgtatttaacatggtaatttcttcaaattggttatgtacactctagtgaaatgtatttatgaagcaacctttggtgcactggtatttttgtgtcttacagtaattacaagtagtggctacagccccagatcagcacatcagtattccccacagctgtatccttccaagtaagtggtcagtagattcttgctttaaattggcaaacatttatgtctatacatgttttatagatggttggataatacttggatagattacatatatagataaatatcctgtacaaatttgaaatggagacacccacatgtatcttgatgattcccttgaatatttatgtataccttgagaaaaatatcaagctcaaattagaatacattgcattttaattcaaattcaaaaatgctggctggttaaaaaatctaaagagattacattttatctaaatgtcaacacttcataattaattccatcattgaaattcttttgttggatttccagttctaataaacatatttttgaagaatatgccttattgtttttataactaatatagtcactttattgaggacagtggtagatatttcttaagtttaactatattgggtgtatatctgaattggtttggaatcagccagacgtgccaagattttgtattgctgatacatacacatttaaatatagtatgttattttctgcagcatctagagtgtctgcctttacatatatatagactgaaggttggatatccaatggggaacatgatgtaaaatgtggcatgatgaaaggaatgatgttccttttttttttttttttttaaggaagcttcctacctttctttttctttttttaaatttccaattcttgggagcgaattcttagtagggcttttaaatagacttccatagtctttgcatagcattaagtcactcatggtgagtgtgaatttgctcatgagcatattaattagctagtgtgtgctccattttcctcatctgtgtattaactggagtgctatttcagatatgagcaagaattgtagacctggaagagacacaggtgtaaaatctaaatcaaatttctacttttacaggtgtggtatagacatagggagtttaaatcatttgtctatggtcactagattggcttgtagcagagcctgagctcctgacattcagactgttgctttttcctatattgttaaaattatattgtggataaagaggattacttcctggatattatagggtttcactgaatctaggctgtctaaattaatacacagtctttgttgccacagtaatgctatttttctgatatttaggccctatccacacattctttctacaccagcagctcaaacaatgtctgcctatgcaggccagactcagtattcggggatgcagcagccagccgtctacacagcctactcacagacaggacagccctacagcttgcccacttacggtatttcacatcttctgttttcttctttggttataggcaggtaatcctgctggctggtagctttgtattctattgtaggttacaacttagaaggaaaatcatcttactaattaaatggtttacacattcaattttctgaacagatttgggtgtgatgttgccagccatcaagacagagagtggactttcccaaactcagtccccattacagagtggctgcctcagttacagcccagggttctctaccccacagccaggccagacaccttattcttaccaaatgccaggtaagtagctacacatgaaacatgttttgggagaactaattctttgagtgagactcattggcttcagactaggaatatccaatcatgaaggtagttttaagctttatcacagctcacacaatttctaaagaaagataactagacagaaacacgatgaaatggttgagtatacgtcaggaaacatgacctataccaaacttaaatacttaagtttaagctgcgtaacatcagcaggaaaccaagtccaagcaaagcaactcccaagccagacaccaggtcagccacacatttggcgtctacttaagcaatttaaaagatctgtagcacaaaccagtattttcttttcactcttcctgaagtatgtaatgctgttttgattatacttaaacttgaattaactcttttctaagttccatatattattgaccagttgaggattttttgttcattcctaaaattcctctctcctattgatactatattgcttaataggactgttatataatatttactctccaatcctccatataggcatataggatcattaaatttttttaaattttagactcctattaaaattgtgttttatctttttttttttttttttttttctgagatgcagtctcgctctgttgcccaggctggagtgcaatggcatgatcttggctaacttgcaacctccacctcctgggttcaagcgattctcctgcctcagcctcctgagtagctgggattacaggtgtgcaccaccacacccagctaatttttgtatttttagtagtgacggggtttcaccatgttgatcaggctggtctcaaactcctgatctcaggtgacccgcccgccacggcctcccaaagtactgggattacaggcgtgagccaccatgcctagctgtgttttatcttaatgcaatagttgccaaaagaattttagtgacatagtaaaaactttaaaagctggttactatctgtcattttgtctgtactgcaaatcacaaatttggggctatattaacatatttgaaccaactggtatgacattttggtcaattgattcaaatgaaataattacttagatatgttcagctggaatagcagtgctcttttttacctagaatgtaggttgaaacatatttgaaagcatgctatagccacaaaaattcatacagcttcagttagccatgtgacagacagtatttttttttttttcacgaatactttcatactggttattccaggtcaggtcagtggcactgattacataatacattgctagtcaaaacacattgataaaacaagtaatcagctacctcaatagcctttcattttacttcttgacaagcacattccataaggactttttttttaggaaacctgtaattggcagctcacttgtttttgccctgatttgagaaatcattaaatttcagaccttgtagtaaagttttagaaggccctcaaaccctagatttgtgtaccataaacagttctcccttgagtctgaaccctaattcacagctatttttgttctaaagaaaagacaaagggaacaaaagtacaaggagttgctaaatggggttctgtatgtttatttcccacgctaaaaaccctgtacaaatgttgaactcaagtgattttttgtatgtaaatgcagttgatatggtttgaaagttccagtagtttcctggaggtagtcttagtctgtgtaagattttatcctttgcatacccctgcacttctagactcttaacccttgaatgttggcctcactccttatgaagactgttcccatgtgcttctgtttagctcaacagattcaataggaaagggtttaccagctgttacaagagggagagagctaagcctattttctgtataaatgtctcaacttcaaatttcactgtcttcacgacaaatttcaaagtgtttctctcacagaattctgcaaagtgaaatatctttatcattttacaaggaatacttttaaaatgcaatttgttttttacctctaggttctagttttgcaccatcatctactatttatgcaaataattcagtttccaattcaacgaatttcagtggttcacaacaggtatagtagctttttgtgtttatgctttttatatgtatttcagatttttgaagagtactctcaggttgccatatatgtgcatactctcccagaataaggtttctttatttaaaaattatgatagcatttcaggatagtaaaaaattagctatctagaattcttggaatatgcattattctggctattttagattttttgattgctaaaggtttgtgctttaaacatttatgctgtagttgttttaatcatttggcatggaattacaaaatgtattggatattttgctatcttaaaatatactaagaacaattttatttttcttgataaagcagttattataatgaatattaattggttgaatcaatattaattgattaaaataagtcataggattatactacagactatataacattttcagtcctaaaaagtgtcctttgttactaaacaaaatgcctcaaagactactgttggcttttaaataatttttctgttttttagttttatgaatacttttaataaatgtttgattcccactttcctgacagctgcaactttttgcttttgtcactgtgcctgcctctggtagtctttagtcctccttctaattatgttttcttaatatttttgtgagttttaaaatctagcatgttagaattatgcatgaagcaagagtgaatgtccctgaataagtaaatcagctcggagtgagatgaattcgtgatatatgttgacacttccaaatcttttgtccttattttttgattttcagccatgttcgaagtagttgagattgaagagttagtggctgacttctggtctggcaatacactattttactttagtatagttgctagacacttatatattctaaaatgtaagttactgtacaagtgcttactttttagatttactgaaggaattttatcaattgctttattttagttttattgatatcacaaaaggcagaaaggaaattttgatcattaaaatcgtgtggaataataatatttttgataggattaaacggtgtaatttagcattaggaaaatgatgtcttaaaacactaaacatcagagccagtgtatacattttattttttaaaattgaggttttttttgtaacttgattttatgcatttatttattggtccagcaagcatttattgagcacttgctatatgttagatttttggctagggattgaagattcaaaacgagtaactcattgttccctgctctttagaaggattcagtcctccgaaagaaacaaatatgaaaataagatggacagagggtgggatgggatatagtgaatataataacagaaatatacactaggcagagttgtactataaaggaaaaactgctctgcctctatttgggaggattaagaaaagatttgtaaaggaaaagagcatttatttgaaaagagttaatcatagaacagatctgattctctgtgaagatttaaatatagtggatcccaatgtaaaatgtaatctgtatttgggaccatggcctgagcaccagtttgttgttttggctcatacctattgagtgcccataaagtgttaggaattggtctgtatactggggcacacgaaggtggggctggagtagatcagatctgattcctgcctcatagagcttacagcctactgggacaaacatacaataaaccagtaaacacacaaataaatgcagaattagaaagagaacagggcctgcgaaaacatcaactgaatataattttgaataggggaggttttatataaggcttctctatggaaaccacatttaggccaagacttctaagagttaggtgaagaaggagggcagtggtgtttcaggcaaagaaagcagagtgtaccaaatccccaatggatggaaaacttggtgtgtcactgaacttaaaaaaaaaaaaaaggctggagttcagttagtgcatgagggggaagtggggaagcacatttgcagagataggcgggggcctgctcacgtaaggcctggtggtttacaataaggaatttggattctattccaagcccagtgggaggctatgatagcattttgagcaagaaaattacatcatttaatttatgttttgagacgataactctgccaccaaggtctttggagaaagtcagaaatggaaacgaggagataaatttaaaaatgaatacattgcctgcaacaggaagaaaaggtagtttggacaagggggcgacagtggaaatgaaaagaaagtgagatctttgagatatgtttgaatatgaaaagacattatattccccagtggggaatagcctggacctttccagtatgtaacctcatcctcttctgcaggaattagtaggctaacagagagcttcagcacagatgtttgacttaggcactctgggtttaaattctagctctgaaacttactagctgtgtgactttgtgcaaatcacttaaccactctgtgccttagtccccttcaaagtaaaatgaatatagtattaagaccacataagagagtataaggattaaatgagtaaatatatctgagcccttagaacagtgctttgatacagagtaagcactatgtaaacatttgctaccactgttttactctgagagatattggtaggaactttgtttaaatatgatgaggtttttgttcttggctcacaagttacagtagtgggaaacattggatcttgaaattttcagactaatagaattttagttttctgccatattacttaatcatttatgaagatatacatatttacttataaagattatttcttttattttttggaaaccacatcaaatgtagatcttttttcctttggaacacatttcatcaggggatcttaatgttatcagtgatgaccaaggatttgtcatcactgataacattttgggctgacacatgctagatatcttaaacaccaattatcacaacaaatccttactcatcatcctactgtgtaaaaggattttgaatcattttaaggttcttaaaatttttaaataaaaagcaggtcaggggtgttgattcagtgctttcatttttctctgtaacaaaggagggaaactaaatcattgaaaagttcactaaagactttaaatcaaggcctcaggtggcatgtagtttgtgagaggctgggtaaaaaaaaaataataatattcatcttcttttatttactttttacctcctgccaagaaaacagaattatcagtggtagcccagaaaggaacaaaaatcacaaaaatgcagactgaggtctctgtggagtctaggaggggcagccccaactccttgctggtgatgttagaagatgtgtaagaacggattttgtgaatatgagctgattccatctccttctttcctcttcaaacacgtaggtcctgatgatgtaaatccttgaataagtaactatcaggagcaaagtgaagcatatgctaactttaatcacattaaaatttgtgtactggacctggtttgccaaaacaatagggtatttcactactatatgtttgtaagaaatgcccacaaagtagtctctgctcattaggatcacctcaaagctgtggactcagaaacaaatggggctgagtactaatctttcagttaccataatgactggttttcttgggagagtattaaagagtaatttctctttcaaacacacacatctcaattattgtttcaggattatccatcctatacagcctttggccaaaaccagtatgcacagtattattcagcatcaacgtatggagcgtatatgacatcgaataacacagccgatggcacaccctcttcaacctctacttatcagttgcaggaatctctcccaggactgactaaccaaccaggtacagatcttcacccaggtgaaatacttttatatgttttgcaatatctaataaaacggagaaagtggacattccaaaaacatggcggaaagctcccagataattgtgctgatgtttaatctgtgttagcatgactgtgtaagacgaggctcttctggcatttcagaagatccttcttttagatatgggacagataggcgattttaatctctaaatgatggtgtacatatgaatatggagcttttgccagactcattattcatttgtgaagaacttgcaacaaagttgtaatattatcttccttatgactgatattatttcttttgtgctaaagagactgcataagcccttcacctttgtaagttgtcagtagctctagcccgtgaacatttcctacattcctgggatttgtagaatcagtgataggtagtttaatatctttgtgtgatcagtgtgtatttaaaatccattgtgtgtttcattatctgaaacttgcagtatttgaattgcatttgcgacacgtaatttgacacagctgaagtatttaaagttaaaagcaaatgtaacagagactcagtataatcctaagaaaagttccaggactttttggagaaattattgaggtgattctaaaatcacaaggaaaggtggatgaattagaatagtcaaaactatttttaaaaataagaacgaagtttgaagactcagattacctgatttaaagacttataatgtacagtaatcaagagagtgcaaaaggatagacacatagagcaatgaaacacaatggagagtccagaaacagatgcatatatatgctagaaaatggatttttgaatcaggatacaaaggcaattgagtagagaatgaatagtttttttcaacaaatggtgctagaacaattggacatctatatgcaaaagaggaaaggatctcaacttatatgcaaaaattaactccaaatggattgtaagcctacatctaaaaagctaaaactataaaacttctagaagaaaaaaatggtggaaatctttgtgaccctgggctacataaagattttatagatatgacattaaaaacacaattcatacaagaaaagaacagataaaattagatttcaaaaaattgtaaagaacttttgctcttcaaaagacactattagagaagtgtctgttcatgtccttcgcccactttttgatggggttgtttgttttcttcttgtaaatttgtttgagttcattgtagattctggatattagccctttgtcagatgagtaggttgcaaaaattttctcccatgttgtaggttgcctgttcactctgatggtagtttcttttgctgtgcagaagctctttagtttaattagatcccatttgtcaatgttggcttttgttgccattgcttttggtgttttggacatgaagtccttgcccacgcctatgtcctgaatggtaatgcctaggttttcttctagggtttttatggttttaggtctaacgtttaaatctttaatccatcttgaattgatttttgtataaggtgtaaggaagggatccagtttcagctttctacatatggctagccagttttcccagcaccatttattaaatagggaatcctttccccattgcttgtttttctcaggtttgtcaaagatcagatagttgtagataggcggcattatttctgagggctctgttctgttccattgatctatatctctgttttggtaccagtaccatgctgttttggttactgtagccttgtagtatagtttgaagtcaggtagcgtgatgcctccagctttgttcttttggcttaggagtgacttggcaatgcgggctcttttttggttccatatgaactttaaagtagttttttccaattctgtgaagaaagtcattggtagcttgatggggatggcattgaatctataaattaccttgggcagtatggccattttcacgatattgattcttcctacccatgagcatggaatgttcttccatttgtttgtgtcctcttttatttcattgagcagtggtttgtagttctccttgaagaggtccttcacatcccttgtaagttggattcctaggtattttattctctttgaagcaattgtgaatgggagttcacccatgatttggctctctgtctgtctgttgttggtgtataagaatgcttgtgatttttgtacattgattttgtatcctgagactttgctgaagttgcttatcagcttaaggagattttgggctgagacgatggggttctctagataaacaatcatgtcgtctgcaaacagggacaatttgacttcctcttttcctaattgaataccctttatttccttctcctgcctgattgccctggccagaacttccaacactatgttgaataggagcggtgagagagggcatccctgtcttgtgccagttttcaaagggaatgcttccagtttttgcccattcagtatgatattggctatgggtttgtcatagatagctcttattattttgagatacgtcccatcaatacctaatttattgagagtttttagcatgaagggttgttgaattttgtcaaaggctttttctgcatctattgagataatcatgtggtttttgtctttggctctgtttatatgctggattacatttattgatttgcgtatattgaaccagccttgcatcccagggatgaagcccacttgatcatggtggataagctttttgatgtgctgctggattcggtttgccagtattttattgaggatttttgcatcaatgttcatcaaggatattggtctacaattctcttttttggttgtgtctctgcccggctttggtatcagaatgatgctggcctcataaaatgagttagggaggattccctctttttctattgattggaatagtttcagaaggaatggtaccagttcctccttgtacctctggtagaattcggctgtgaatccatctggtcctggactctttttggttggtaaactattgattattgccacaatttcagagcctgttattggtctattcagagattcaacttcttcctggtttagtcttgggagagtgtatgtgtcgaggaatgtatccatttcttctagattttctagtttatttgcgtagaggtgtttgtagtattctctgatggtagtttgtatttctgtgggatcggtggtgatatcccctttatcattttttattgtgtctatttgattcttctctctttttttctttattagtcttgctagcggtctatcaattttgttgatcctttcaaaaaaccagctcctggattcattgatttttttgaagggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtaggcatttagtgctataaatttccctctacacactgctttgaatgcgtcccagagattctggtatgtggtgtctttgttctcgttggtttcgaagaacatctttatttctgccttcatttcgttatgtacccagtagtcattcaggagcaggttgttcagtttccatgtagttgagtggctttgagtgagattcttaatcctgagttctagtttgattgcactgtggtctgagagatagtctgttataatttctgttcttttacatttgctgaggagagctttacttccaagtatgtggtcaattttggaataggtgtggtgtggtgctgaaaaaaatgtatattctgttgatttggggtggagagttctgtagatgtctattaggtctgcttggtgcagagctgagttcaattcctgggtatccttgttgactttctgtctcgttgatctgtctaatgttgacagtggggtgttaaagtctcccattattaatgtgtgggagtctaagtctctttgtaggtcactgaggacttgctttatgaatctgggtgctcctgtattgggtgcataaatatttaggatagttagctcctcttgttgaattgatccctttaccattatgtaatggccttctttgtctcttttgatctttgttggtttaaagtctgttttatcagagactaggattgcaacccctgcctttttttgttttccattggcttggtagatcttcctccatccttttaaaaagacaagctatagacttggagaaaatatttataatacacatactgatacaggaattatataaatatataaagaactgtcaaaattcactaagaaaaattaggttaagaaatttttaaaatatcattaatttgttcaagtatatttttctgccttcagttatttgcaagatccaaattcatctctacttctttcttattcagctttggatgggagggaaaatagggaaacatggtcacagtactgtaaactctttctaactggaccagtggggccaagatgactcaatgccattaaacgattatttattgagtatatattaggcatcagcactgtgctagaaactgatatttcagcagtaaatgagctctttgcccacctttggctttcatttgacaagcaggacaaatatcaagcaagtaattacaatagtgagaaagggaaaatggtaggtcatatgggggatattaacaagtaaactaatttggacataaagtaaaaatgaaactgatattttaagcaacagccaagatatgtttatatcatgaattgatgaaatgataacattcatgatttttagttccctgttcagaatataatctttacctgtgggtccaagaaataattatctagtagatagagcttgtctttgatgcccctacattttagttttttccccttatcttccttttatatcctcctacctgtgggcacacagcccctttgaatttctggtccttgtccctttggggctctctgttttggcttttcagctagccttagcctccctgggtatctgtaccccttgcagggccaggattagggtgaggtcagtgaagcacctagagcacaaaatttaaggtggaactcgttcttggggtccttaaattttgtgccctaggcgccttactcattagccccagcccaaatcacatgttccaaatctgtcatgatcctctttgggtcaatacttgtgttgcatcaaggatcttactttcattcaaagaatgaatgatttcagaatccttgcttaacaagcagtggagatacttcagggaaggagatatttcacgacctcttatcaccctgtggaaggtttttctcaatgatgaatgtgtgaggaaaacaaggtagggtgaaaccacagaagtatttaattatcaagatatcagagactgaatgatggacaagaccaaaagggtgtttttttaatttgctagactacagctaccaagaatgaaacccatttgtgtactcagatacggtatatgctacgtggagctttgtgtaaaatgttgcttttgctccaatttccatataatcacgagaaagcttctctgcaggaaaaacacagatggttactttccttattagaataatcagtattgaaaatagctattttattcctattaaatatattaaatgcaggcaggtagatattctgagtatgtgactttggatgtgacacattcacatatggaattctgacattcactatcagaatgctgcaatttcatcaggccttcattggttgctgaggtcaagaaatatagtttgacatcattttcagattttattagactgtctgttgaatttaaagaaggaattaattctacttataactatattattctctcaaccaaaatcgtaacaacacacatgtctggatctttaaagaattctatgacctcagaccttcagtcaatgcagttttaggtttaattttgccttttcagatctggcatacggccaaaaagcaaacaagtttataactatggaatttttctttattattgatggaatctgatgtatttagttcaagggcaagaaaagtatttcccattgactggttctgcattttggacttaaagtagaaacctaaaattaaacccacagtttttcatagaacagtcactgctgaattctacttttataatgaaaacataccttatgttcaataagagacagctggtttttctgatttagagataagcctaatgaaataaagtgggatctaaatatccaaaacttaaaaggtgcagttattcattactgtactgattaaaggataattattcaatcatgtatagtattgggtcaagaaaagcctagggcaattttgtaatgactgatgttacttggaaattgcaaatagcattccattatttaatttaccttattaggtagtttgacagtggatttaaagaaacactaccatgctgattaagagcagacattttacttcattctctttctaaaggtctttctttaaagtaaaatttatattgggtatatcagattttttatttgtattaagaaagtttgaggacagatttgcacatgaagatgagaaatatttttatagcgcctccttttcaaatattttaaacatctttaaaatcaaatacagttagtcttgtataacaatattatgttttaccctgacactttgacaaacattacaatacattagctaactgaacctaacaggtgctctagaaaagtaaaggatagagtaagatcatttcactctctgaaaagaagccagtttcttaggcagaacacagcagccttttgataacttcatggtatttcacgtaaggaaaggaagggaagtaaagtgctaactcatctaatggaaactaacaagaggatttagatagaaaggaccacattcaactgctctgccaatttacattttccctacctgtggcattgctatgcccttgtgcaattacctgagtgaaatttggcagaggcactggaattaacagctttcttcttttaaaaaaaagtgtcaacaaatgtttaatgactggttgagctcaagttcctggttttaccctttatcagaagaaaggatagcacctgcaaaaggaatagaggtcagtacaattccacctgggtgattgctctaggagcccagatgtatcgcatggcctctgcagggcatgctcttttgcttctgtgaagttctttcagtgaattctttggccctccatctcacagaaattatataatgcccatgtgacttttattttaatttattgtttaactgacaaataaaaaagtatatatattggtgcagtgatgggcacccgtaatctcagctactctggaggctgaggcgagaagattgcttgagtccaggagtctgagtctttaaaaagaaattgtatctatttttgtgtataacccattgttttgaaatacatttatattgtgaaatggctaaattgagctgattaacatatgcattacctcacatgcttatcattttgtggtgagaaccccgaaaatctattctcttagcaatttttagaaatacaatacattgttactacgtatagtaacatgttgtataatagatatcttgaacttactcctcttgtctagctgaagtgttatatcctttgaccaacagctccccagtctccctctccagccccacagcccttggcaaccaccatcttactcacttcttctacgagttcaacttttttgcactccacatataaatgaggtcatgtagtatttgtctttctgtgcttagcttatttcacttaacataatgttctctaagttcatccttgttgtctcaaatgacaggattttcttttttaaggccaaacattattccataatgtacatataccacatttcctttgtccgtttatccattgatgaacacttaggttgactccatatcttggctatttgtgaataatgctgcaatgaacatggaagtgcagatatctcttcaacatactgatttcatttcctttgatatatacccagcggtgtgattgctggatcatatggtagttccgtttttaggaacttccatagcattttctataatggctgatatggtttggtggtgtccccatccaaaatctcatcttgaattgtaatccccctaatgcccatgtgtcaagggagagaccaggtggaggtaattgaatcaggggggcagtttcctccatgctgttctcgtgatagtgagtgagttctcatgaaatctgacagttttatacatgtttggtagttccttctgctttccttctccttcctgccaccttgtgaagaaggtgccttgcttccccttccccttctgccatgattgtaagtttcctgaggcttcctcagccatgctcaactgagtcagctaaacctctttcctttgtaaattacccagtctcgagtagttctttatagcgatgtgaaaatggactaaatggctgtgctaatttacacccccaccagcagtgtgcaagatttctgtccgccacagtgtctcctacacttgttatttttcttcttttagataataccattttaacaggtgtgaggtggtatctcattgtagttttaatttgcatttctttgataatgagtcatgttgagcatttttcatacacctgttgtcatttttatgtcttcttttgagtgatgatcatgccaacccattgcccattttttatttgagttatttgttttttactattgagttgtttaagttccttgtgtattttggatattaaccctttatcagatgtatggtttgcaaatattttctctcactctagtaggttgttatatgactcttttataagtagaagcagatctaggtttgaccttctggtaacctgaagagtccattgctatttgcctgagaaagtatagccaaatggttaaaggaggagttctgcagtcataccagtgttgcatcatacaagctatttaattttgttaaattaattgccacttctccagtttctccattattaaaagacaataatgaaggacctaattcaccgggttggagtgaggtttaagtcagacacacacacacacacacacgcatatatatatatacacacataaatatatataatgtttcataataaagagctttgacaaatcaataagaaaaagacaaacagcctagttttcttaatgggcaaaagatttaacagttacttcattaaaaagaagatatcaaatgagcaagaaacatgaaaaagtattcttcataattactcatcaagaaaattataattaaaaccacagtgagataccatcaatcacacacccaaaatggtgaaaataaaaaaacgctaacaatatcaagtattgacaaggatgtggaacaactgaaactctcattgtggtaagagtaaattaatactgtctttaaattaataaaaatgagcagtgggttagcatggtcgttaatacactggaaagagagtaatacactggaaaaaagtcagtgtctgttaaagatgaacatatgccttccctctgacccaggaatcacattgccaggtatgcatacccaagaagaattaagagcttgtatccacaaagacatgtacaagaatgtatatagcagctttattcatagtagcctaaaattgtgaataacccaaatgtccatcaacagaatggatacagtgtaatagatacagtgtaatatatatatacatgaagaagttctagccacattaaggaaaggacaactgctatcccaggctagaatatggattatctcacagcagtgaatgaagccaaacacatatatttcaataaaaaaaatttttaaagaacagtgcatgacatgtactaaggggtgaataaatgtagagttacccttcatagttttctaaaaaggctgtctttatccccatcaaaacacttctgcagatattttgtccaaattcttagagcccttttaagatccttcagtaatttgaaaagcatcaatgggaataaagtcttaggtttactgaaacccagatacgtcctacaaccacagtcttttctctttcctcttctgctcttttcacccctttgctgtgacagctgctggatcccagttcatttgaatagcagaattattcctgctaaactcctcctttatggactcagcctacaaccacccatgactggggttcctcaagtttttgtcctttcagctcttgcactgtgacaagaacaaagttgccttatgatagttaacacctgaaaattgatctataatgactataaggagaagttttatggaacgtcatccaaaatgttttttaaatgcatgcttaataagtgagggttaattttaatcaccattttatttgaaagcagacatttggtttgtgggatatagtaaccttaggaaaattctgtttcaaaaataattaaaaatggaacttttatgtgattttatttaaaagttggtgtatgttcagacatctaaagactaatttaatgcctatacaaataaggtgaaacatgatttactatgagacaaggctatattcaaccccataacttaaaaaccttattactctgctatctactctcaccgaggtgataacatagacccatgaaatgaactatgctggcaatattttaagtgcaaataatatttggagcacaggcatatttttttgatcatgtgctgacatgcacagtgtttcccaaagaaaatgttagaggttacccatgtaaaaattactatttctcagaaaaagtgatgcctagtttgttcagatttgtttatttagattaataactgcttggttttcctcagaattgttttattttaatttttaaatttaatgataaaaaataatagccataatatctttttgtttaaaaatcatgtaatttaaatttaatgtagtttaccttctgtcttggaaattgtcactttatacatataatttaaatcaaattaaatataaatcagatttatccctcagtattccagaaagctgccatgaaactgagatttagagagattgaataaaatgcttgtactcatatagtaattctgactcttcaacttgttatactgtatctcacagttgcctcagtttatcttaacagttgctcctacagatttgctactactcagtactctctacctcctaagatactttctcacaagctttaatcatttcagagttttagatatctatgggacgtacagaagtcaattggattctgaaactgaattaaagtctgaaacttaggatgtggcctagttggaggtataggtttatatgtgagtcatcacagagagggtcattaacacatgagagagttaaatcccccagattgtgtggagtgaagacagtgatcagaggatggaattctgacatactagctcataaggaatgggagaagtacaggagtccatgttttctagatggagatggactgatcggagtgattctagaactaggagagtgcaaggtatcagctgtcaaataatagtttcgaggaggtgctgtttaagagaatcacacacagcacagagatcctggaagacaaggactgagtctgtctattgcatttgtcaacaaggtgatttctggggactattagcaggagtatttttatcgcattttggggaactgaagctagattatgataaattggatatcaataagtggggacagatatgccgtccagtgctgcagccactggccacacgtgactcttgagcacttgtcatgttgctagtgtggactgagatccagtataagcatagaacactgcacaacatttcaaagacttagtatgaaaaagaaaatgtaaaacatatcattgataattttatattgattacatacggatataacatgaatatattgagtcaattaacacattgattacatactgatatgataacatggatatattggggcaattaacatagaagtgatgataattttacatatctctttttactttttttaacatagctgataaaaaactttaaaatattatataagatcacatttgtggctcacatggtatttctgtcatatagcactagtatgcactattctaagaagttttatgtaaaagagaaaatggaagcaaatagtatctagatggaaattccaggtcagagggaaggactttttaacttctcagtgaccccaagaaatgggagaggtggaagggaaaatgggagaggtggaagggaagatgggagaggtggaagggaagatgggagaggtggaagggaagatgagagaggtggaagggaagatgagagagggtttgactaatgaagcatgacttcctaggaggtggggctgggagtcaggtttgcaggtggagggatggaattgatccagaagaagacatctcattctctgaactgtgacaaaaaaggaggtgaggacatatctaaatggagataaatatgtaagtgaaggtatagacaggaagttgaaggcgtatggtgtagaaaagacattcaagtttaatagatcacatagattacaactagtaggagtttgtgtaaactacaaaagttctgactgacttgtgtgtgcttgggattataaatctcttttttgccatcaggaggtttctattgtataggaatttgttaagataattaataatgaagactcatacttgatctaaaaatgaagtgctattcttgacctaagtcatgttatctataggagagttcgataccatgcagagtccctccacacccatcaaagatcttgatgagagaacctgtaggagttctgggtcaaagtccagaggaagaggccggaaaaataatccctccccgcctcctgatagtgacctggaggtatgcctactcattcttaaagattgtagtgtgatgctttattttaccgaatgatacattctctatcttacagttccattatgtttcaaatttaaaaatcaagatatatcatgaattgaatggaactactttgtgataggcattttacaactctgaagtttgagattccatccattataatattaaaattaagaacaaataacagtataaaaatgatcttgatttacagattattggcctgagataattgcaagttttgatctaaattggcaccgacaaattttaaaactatagccattgtttgtgggatacttttctatgtagcactgtattcgatagcactaaagtaatggaagatgtccaaatgggtcattaagtttaaaaagatgaaagtaatttgtatagaaagaaaaattcatcttctaaaaataggacaagtttcaatcacttaatagtatgtgttaaaatctgaaaatttcattatatgtcactttctatttatgagttcttttgagatatatctgtactaagcagggttgatttttatatacaacatcagtagaaactattcttaaccataatatgttacttcttacataccacatacttcggttaagaaaaaaattgaacttccagatttagtaaaatcaagtgacagacgttgtgattctttataagagataacttccttttcttgaacaacagcaacaagttcacattgcctcaagaacttgacagatgatataaatgagtgaccgggctgagttgcccagcagactgtgaggagctcagagtgcaggcccattctgagggacctgcctgtcactgccatgtaggaatggcagccagcctggccacaacttctagttgctgtgctacagtcaaaaccctgaagtttgtgggcaattgcctagttctcaaatgttaattcataatttgaaatccttcaaaactccagggaggccaacatatctgctggcctaatgtgaccccaggatctatacttgacaattctgtttagcggttactcattgattcttttaaatatttctttaaaatctaattctttgcagaaattgtaaaagtttatgaatccctaaggtataaaactttaaaatatgtagagtttcaagcatgcttctatcataaagtctgaatgtatctctttttggtgttaactacgagtaaatgtatggattaatttattcaagcgaggacatctgactcttaagcttataatttgtataattcctgctcctcaggctttataggagttagatttggcaaatttgaaaaacaaaaaaatgaatagtattattttctattaaatgatatcaagatgatctctaggaagggagacattttctgcttgtaaagagatttctgtttccttggactttttaattttctgatatttattttttgtcttcagcgtgtgtttgtctgggatttggatgaaaccatcattgtttttcactcactgctcaccgggtcttatgcacagaagtatggcaaggtaagaaatcaagaaatgttactccaagaaatcttgttaaattttttgtgtttgtttaaaatcaagggctatttaaaaattcttttaagtgtttttttattgcagtctgtgaaatcttctcttaattatctatttctagtgtcttatagttctttaatatgaactggcaaatttaacaaagctaattataaattgaattccttgctatttcttcaaaaggactgagttttctacatcaagaccaagcttcttagtctcacataattattttagaaaaaaaattgtaaatcgaaatggacttcaaaacattataaaatcatatattatccccaataaaagagctttaagaaattgtttaaattaatttgttgcaattttatagcaatagcgagtaatctttcgtctgacagatcttttggatttatgaatgagtgttttttctgtgacttgagtacacatatgaacagcaaccgaggtaacaccccctcccatacacaaagtcagcaatagatggtctcatgcctcacctgcctttctctcttgctattgtttttattttctcttttatttatttatttcattgttattattattattattattattattattattattattattttgagacaaagtctcactctgtcccccaggctagagtgcagtgacatgatcttggctcactgcaacctctgcttcccgtgctcaaatgatccacctgcctcagcctctcgagtagctgggatcacaggtgcccgccaccatgcctggctaatttttgtatttttagtagagacggggtttcaccatgttggccaggctggtctcgaactcctgacctcaagtgatctgcctgcccctcggcctccaaagtgctgggattacaggcgtgagccaccacacccagcagttatttttatagttagcttcttataacatcattattacaaacctctgtgagtgcttctccgaagactgatgcaactctgaaaggctttttggctagaaacacagctgtttaggcaataactgtttaagtcttagactctgtagggctcataatcaaactaaagccaactcagaacatgttggcttactgtcagttactatactcaaagttgtatctttggttcctttacatatcacgcttacagaaatgtcttcctatgtcttcatagcattccattattttataatatctcatgctcatgcagtttttagctttgtctatattaagctatattaagctttgtcttctgacaagtaatgataaaattgcgttgaagtctagtcttcctaaattacactttgggtgaatctgcattgtcatttggttttagtataggtaatgaggtaatggatatttctaaacagatgtgaaggtaataattttgtttaacaaccatttacaagaagtgttagcccctttatcacaaaagtaaagtaaattatacagaaatttggagtacacactcaatatcctactactgattaagtgtgcaatataaaaagtttccagactataatctgaagaatttattctaagtagtattggcttatcagaagtacacagtaaatcctgttgagctgattgcaatatttttgtgcagctaagtttcatattaggtaagtgtttaaaataaacaatctaataagcagactggcccaaataaagatcccttcaaaatagtcaccatgaggtgttacacctaaatatccatgttgcccccattgtgccaaatcagtggtagaactcatcttttggaattatcattagccttttaacatatttcctggcttcacatctttatccttttgtggtcattttgatctttctttaaaaatagaaagatcattcggacccaaggcggtgaataaggagcatggtaaaactgtcactgtatatatttgttttatttttgtcaaaaataccatagagtaatgaagctgcctttctcaatttactcataaactggctttaagagcaattccaaaagcaaagttcaacttctctcagcactgctgcgtcatggaaataaacctgtcagctcccaggatgtcgacaggacatgcagcactcatttcaacacacaagttttatttttccaaagtatttccctaaaaggttttttttcaaattcaaaatagagactctctatgcatcctctgtgcttctagaaggttaaaaccaaaagaaggagaataccaaaagggtggggcaaagcgaggggaagaaggaaactaaccattatggaaggctcatgatgcgccaggcaggaagcatcccacaggatgaaggggtaggctgttctctcaaagctatagatgaggaaacatatccagagaagtaataaaaatgtgtccacagccacacaaccagtcagtggcagagacgagtcctggggccccagattctttgaccctgaagccatgttctttctgccatagcacatcgcatggcctggacagttaatataccaggaatgcaatagaaaagtaatatttccaaacaggagtaaagacaattagtgaggtactgcataggcagttgggtgagttctcttttaagttgtaattttaatgatcttctctcagacacttggaatgtgttttcatcaagaggtaaaacttaagaaatattccatgaaatagaatatggcagacagtaatcagtatctgctggtggggcagaatacagaaagaatcagaggcactattggaaggggatgagaaagataaacttacatttataggatacctgtcctgtggttcagccgatgtttgacattttagaagcaatagggtggtggtgaaaggcaaaagcttttgagtcagataaacttaagttcgaaaatcgagttctgatgcttacagcattctgactctggcaaattacttaaatcttcagtttcaaaatctgtaaaatgggaataacaccatctgttagattgaccacactgaataatgtttttactcagagcaatttaagaaaactaaagtaaaagtgtttttaggctatattttttttaataaaaacaaaaacatccttgtcgaattccatgaaaaacaacttatattgccttatttattgaattattacatatatttctctaaaagttgttttatcattgacaatttacttaatgtaattcagaaaatcggcactgccttttagccagtaataaataatatcttggcatattgagaagtagctaaataaggcccatcttatgaagctatggaccacagacacctcagggttcacggcagatgggacagagatgacgttgggtagcttcagcttcaccacaaaccataactgaaaaatggtaaactagagcccctcagacagactgcattcatttaacaaatagttagtaatatcaatagacatgcttcttgctctcatggagcttacaaaccaaacaaaaaacaaaacaaaacaaaaaaaaacacatttcaggtattggtgacttctgtgaagaaaataaaactaggtcgtataatgagtgactggggtatgtgactgaggaagtgacattggtactgagaggagctagccctacaaattaagagagtgggagaattggaaagagcattccagcagtgggtgctgatcttccagtgttccaggaatatgaagtctagtgtggctggaaggttgtaaacaagagaaagaatagtacaaggtaaagggaaggaggtggaacaagttggcaaaatagaaacctccactgattgtcctccctgcagcaacaccaaattgaataagtatccacacaacacaaccaccttcataagaaccaaaagccaggtgagcaatcacacaatacctagttttaactcatattactgaaggaggcactgaagagggtaagaaagacagtcttgacttgctgacaccaccaccccacaccagtcttccagcagcagctgaatggcacagagagagaatctgtgcacttgggggagggagagctcagtgagtgtaggtttttgcattggaactcagtgacctttcacagcagaaagcaacactgggtagaacttagccaacacccatggagggggcatttagaccagtcctagctagagaagaatcttccatctcagctgttggaacctgagttctggcaaatcatcaccaccatgggataaagtgctctggggtccaggcagtttaggccacaaggaccgcaattcctggacaagtcctgctgctctgctgggcttgaagccagtggactcggcagacacatgacatggtaagacaccagccagggcagccaagggagtgcttgtgctacccctccctcaggcccaggcatcacagctcacagttctagtagagatgtcttccttccacttgagggaggagaggaaagagtaaagaggaatttgccttgcaacttggatactaactcagtcacagaaagaaagggcaccaggcagagtcctgaggctccagttccaggccctggttctgagataacatttctacacacgccctgaaggaagcttgcttccttgaagggaaggatccaatcttggcaagattcatcacctgctaccaagattcatcactcttgggccctgaataatcaacagtagtagcaaggcagtacttgccgcaggccttgggtgagactcagagatgtgctggctttaggtgtgacccagcatattcctagctgtggtggctatggagagggactccttttacttgagaaaaggagaaggaagaataaaggggacttcctcttgcagtttaggtaccagcttggcaacagtgggatagggcaccaagtcggcatgtggggtccctgattccaggccatggctcttggatggcatttctgcacctgccttaggtcagaggggtgcccactgccctgaagggagagccctaggcatgaaagcattcgccacaagctgactgaagagcccttgggcattgaatgaacatctgtgatagctacacattacttgccatgggcctggagcagtcaatagtggtcatgggagagaattttctgcttgtggaaagggtagggaaaagtgggaaggacttcattttgtggcttgggtgctagctcaaccacagtagaatagagcaacaggtagtttcctaatagagcaccaggtagattccagattcaggtcctagctcctagatggagccaggctctgtagacccaccagggaccaggggaacttgacaccctgaagggaatcatacaaacttggctagttttgccacctgctgattgtagagccttgagcaaacataggtggtagccaggcagtggttaccacaggctttgaatgtgacccagtcctgtgctggctacaggtctgatccagcacagtcccagggtggtggtcacagaggtgcttgtgtcaccctttccccagctccaggcagctcaacacaagagagagaccccatttgtttgggagaaagtaagggaatagaacaaaagtctctgcctggtaatccagagaattctttgagatcttatccaagaccaagaagagggcatctctatgagtctgcaagagccacagtattactgggcttgggtgcccccatttaatgcagatagcatacagtgaccaaaaacttagatcacaacacccaagttccttcagatacctcaaaagccttcccaaaaaggacaggtataaagaagcccagacagcagcgactccagtaaatacctaactcttcaatgttcagacactggtgggcatccacaatcattaagactacccaggaaaacatgacatcaccaaacaaggcaccagtgatcaatcctagagagatggagatatgtgacctttccgatagagaattcaaaatagcttttttgaggaatcgcaacaaaattcaagataacagagaaggaattcagaatcctataaaataaatttaacaaagatattgaagtaaaaatcaagcagaaattctggaattgaaaatgtaatggacatattgaagaatgcatgagtctcttaacagcagaattgataaagcagaaaaaagaattagtgagcttgaagacaggctatttgaaaatacacagccaggggagacaaaagaaaaaagaataaaaacgaatgaagcgcacctacaagatctagaaaatagcctcaaaagggcaaatctaagagttattggccttaaacaggagatggaggggtggggctagaaggtttattgaaaaggataataacagagaacttcccaaacctagagaaagatatcaatatccacatgcaagaaggttctagaacaccaataaaatttaacccaaaaaagactatctcaaagcatttgataatcaaacctcaaagcccaagaataaagaaaggatcctaaaatcagtgaaagaaaagaaatgaggaacatacagtggagctccagtacatggcagcagacttttcagtggaaaccttataggctaggagggagtggcatgacatttttaaagtgctgaaggattaaaaaaaaaaaccatatttaccctagaatagtatatccagtgacaatatccttcaaacatgaaggagaaatactttcccagacaaacaaaaactgagtgatttcatcaacactggatttatcctaccagaaatgctaaagggagttcttcaatctgaaagaaagggatgttaatgagcagtaagaactcatctgaaggtaaaaaactcacaggtaacaataagtacacaggaaaacacagaatagtgtaacactgtaattgtggtatgtaagctactcatactttaagtagaaagactaaaagatgaaccaattaaaaataataactgacaacttttcaagacatagtaaaataggatataaatagaaacaacaaatagttaaaaagcaggggaacagagtcaaagtgtagagtttttaattagttttctctttgctggtttgttagtttgttggtttatgcaatcagtgttacattgccatcaatttcaattaatgagttatattatttgcaagcctcatgataactgcaaatcaaaaacatacaacagatacaccaaaaatacaaagcaagaaattaaaacataccaacaaataagtcatctttactaaaaggaagacaagaagaaggaagaggaaaccacaaaacaaccagcaaacaaataacaaaatggcaggaataagtccttacttatcagtaataacactgaatgtaaatggactaaactctccaatgaaaagacatagagtggctgaatagattaaaaaaaaacaaaaaaaaacaaaaaaccgtgacccagcgatctcttgcctacaagaaatacacttcacctataaaggcacacagagactgaaaataatgggatggaaaaatatatttcctgaaaatgaaaactaaaaaagagctagagtagctatacttataatcatcaaagtggatttcaacacaaaaagtaaaacaaaaaaaaactataaaaattagacaaaagaaggtcatcatataataataaaggagtcaaatcagcaacaggatataacaattttaaatgtatatgcactcaatactggagcacccagatatataaagcaaatatcattaatactaaagagagagataaacttcaataccatagtcactggagacttcagcacctgactttcaacattgggcacatcatccagacacacaatcaacaaagaaacactggacttaatctgcacaggcttagaccaaatggacctaatagatatttacagaacatttaatacaacaactgcagaatatacattcttctcctacacacatggatcattctcatgatagaccacatgttaggccacaatacaagtctttaaaaattaaaaaaaatgaaattatattatgtatcttctctgaccacaatggagtaaaattagaaatcaataatgaggaattttggaaactgtgcaagcacatggaaactaaacaataactcctgaatgaccaatgggtcaatgaaaaaattaagaaggaaattaaaaatttcttgaaagaaaaaagtgataatggaaacacaaaatactaaaccctatggaatacagtgaaagcagcactaagaggaaagtttatagctataattgtctgcatcaaaaaaagaaaaacttcaaataaaccacctaatgatgcatcttaaagaactagaaaagatcagagaagaaataaatgaacttgaaatgaaaaaaatacaaaagatcaacaaaattaaaagttggttttttgaaaagataaacaaaatagacaaatctttaaccagcctagctaacaaaaaagaagactcaaataagtaaaaccagaaatgaaaaaggagacgttacaactatacttcagaaattccaagtattcttagaggctactgtgagaaactacatgccaataaactggaaaatctagatgaaatggaccaacaactagacatataaagctgtcaagattgaatcatgaagaagtccaaaacgtgaacacaccaataacaagtagagatggaagctgcaataaaaagtaccccagcaggcccggcgcagtgggttatgcctgtaatcccagcactttgggaggccgaggtgggcagatcacgaggtcaggagatcaagaccatcctagctaacatggtgaaaccccgtctctactaaaaaatacaaaaaattagccaggcatggtggcgggtgcctgtagttccagctactcgggaggctgaggcaggagaagggcgtgaacctgggtggcagagcttgcagtgagccgagttagcaccacagcactgcagcctgggggacagagtgaaactccgtctcaaaaaaaaaaaaaaaaaaaagaggaagaagaaaagaaagcctgggaccctatgactttactgctgaatttttccaaacatttaaggaagagctaatagcaaccctgcccaaactattccagaaaacagaggaaggaatatttccaaactcattctacagggctattgttactctgataccaaaaccagaagaagacacattaaagaaagaaagctacaggccaatatccttgataaatattgatgcaaaaatcctcaacaaaatactagcaaactgaattcaacaacacattgaaaagataattcatcatgacccaagtgggctttaccccagggatgcaaggatggttcaacatatgcaaatcaatcaacgtgatacatcataccaacagaatgaaacacataaagattatttcatctgatgctgaaaagtatttgataaaattcaacatcctttgctgataaaaaaaatcttcaaaaaactgggtatggaagaacaataccttaacacaataaaagccatatatgacagacctgcagctgatatcacacagaagggggaaaaattgaaaaccgttcctctaagatctggagcagaacaaagatgcccactgtcaccactgttattcagcatagtactggaagtcctagttagagcagttagacagtagaaagaaataaatggcatccaatttggaaaggaagaagtcaaattagccttatttgtagatgatataatcttatttttagaaaagcttaaagattccatcaaaaaactatcagagctgataaaatcagtaaagttgcaggatacagaatcaatcaacatacaaaaatcagaagcatttttatatgtcagctgtgaacaatctgaataagaaataaaaaagtaatgctatttataatagctacatataaaataaaattcctaggaattaaccaaagaaatgaaagatctctacaatgaaaactgtaaaacattgatggcataaattgaagaggacacaaaaaaaatggaaagagattccatgttcatggactagaagaatcaatattgttaaaatgtccgttttacctgaagcaatctatcaattcaatgcaatcactatcaaaatactactgacattcttcacagaaaaaaacaatcctaaaatttatatggaaacacagaagacccagaatagccaaagctaccctgagcaaaaggagtaaaactggaaaaatcacattacctgacttcaaattataacacaaagtgatagtaacaaaaacaacatggtactgtcataaaaacagacatataaagcaatgaaaagaatagagtacctagaaacaaattcatacatttacagtgaaatcattttttacaaagatgccaagcacatacattggggagagaacagtctcttcaataaatgatgctgggataatggagatccacgtgaagaagaacgaaactagacccctatctcttgccacatacaaaaatcaaatcaaaatggactaaagacttaaatataagatctcaaactataaaactactacaataaaacattggggacactctccaggacattggactagggaaagatttcttgagcagtaccccacaagcacaggcaaccaagcaaaaatggataaatgggaggcatcaagttaaaaagcttctgcacagcaaagaacacagtcaacaaagtgaagagataatccacagaataggagaaaatatttgtaaactatccattatacaagggattaataagcggaacatacaaggagctcaactcaataggaaaatatttaataatctgaataaaaaccggtcaagaaatttgaatagatatttctcagaagacatacacatggcaaacaggtacgtgaaaaggtgttcccatcattgatcatcagagaaactcaaaactacaatgagatatcatcttaagacacttaaaatggtttgtatccaaaagacaggcaatgccaatgaggttgtacagaaaagggaaccctcatacaccgtttgtgggaatgtaaattagtaccaccactatggagaacagtttggaggttcctcaaaaaccaaaaatagaactaccatgtgatccagcaatctcattgctaggtatatacccgacagaaaggaaatcagtatatcaaagaggtatttgcactcctatgtttatttcagcacttttcacaatagccaagatttggaagctacctaggtgtccatcaacagatgaatggtaaagaaaatgtggtacattacacagtggagtactattcagccataaaaaatgagatcctgtcatttttagcaacatggatggaactggagctcttttagtgaaataagccaggcacaaggtgacaaactttacatattcttacttatttggaagctaaaaaacgaactaatggtgacagagtagaacaatggttactagaggttgggaagggtagttgttgaggggtgcagttagtgggaatggttaatgggtacaaaaatatagttagacagaatgaataacatctagtatttgatagcacatcagggtgactgtagtcaacaataatttattgtacattttaaaataactaagagagtccaggcacagtagctcaggcctgtaatcctaacactttgggaggtgacgcaggagaatcacttgaggccaggagttggagattagcctggacaacatagtgagaccctgcctctacaaaaaaaaaaaaagtgttttttttttaattagctgggcatggtggtgtatgcctgtattctcagctactcaggaggctgatgttggaggatcatttgagctcagagattgaggctgcagtgagccatgttcacaccactgcattccagcccagatgtcagagtaaaaaataaataaaattataaaataaataaaagagtttaattggattgtctgtaacacaacaaaatgataagcacttgaggtgatggataccccatttaccctgatgtgattattatgcattgtatacttgtatcaaaatatctcatgacgccaggtgcggtggctcacgcctataatcccagcactttgggaggccgaagtgggcagatcatctgaggtcaggagttcaagaccagcctggccaacacggcaaaaccccgtctctgctaaaaataaaaaaattagccaggtgtggtggtgggcacctgtaatgccagctactcaggaggctgaggcagcagaatcgcctgaaccccagaggcagaggttgcagtgagctgagatggctccactgcattccagcctgggcaacagagtgagactctgtctcaaaaaaaagaaaaaaaatctcatgtatcccatgtgtatatacacctactatgtacccacaaaaataaaaaatactacagagatatatttatttatttataaataaatataaatatattcatttatttataaataaatataaagaaatatattcatttatttataaataaatataaagaaatatattcatttatttataaataaatataaagaaatatagaagagagaggtaggcaagggctggaccaggcaaggactcttcattatggtaaggtgtttgggttttttctggttgcagtgagaagctgttggggagttttaggtttattattgtctgctatgcagagaatagaatttagaggaataagagtggacacaagttgaccacttaggagacactatgcagtaagaatggccatgccttagataggaacagtgcttctgaaacttgagtgtgtgtcacaataacctgcagggcctgttgagcacagattgctgcgttacaacccagagtttctgattcaagaggtttgaggtgggaccataggttttgcatttggaacaaatttccagataaagctgatggtcctgggctgcattttgagaaccactagagtggagtatttcccagactttttttccttgagcaaactaatgaacatgttaattaatagtctttgagaaaaggccatggtcaaatatatttagggaatgcttacattttatagccattcaatgcatgttatcatattaaaggtcatgagatgtttaaaagaatcctctataactaagtttaatccctcagttccctaatgtcatcagtaataaccttgaggactgactgaaataaggttattctgatgattagttaagtcaggactttgcattgctaataacaaaagtgaccagagccattatttctgggaggggggaaaagacctttgtagtgttaagattgtctaagcttgaaattattatgggtccttaaaattattatgggtccttaattcttttcatttcttagatcagtcttatttccacaatatgcatgttagagtgtaacaacattttggtatagcatagtgatttttttgattattttagccagagattcctctacacacatatgaccgagagcatcactgtatagaacatctaagggcagaactgctctaggtaaaatgtacagatgggggactggagagatcccatttcccctacatgaccacccttccccatcattcttggtcaccatgaaaggtgttcctgaatcctggagcttgctcagaatacagcctggaaggcagtcgactagctgaaggagcactgaactaaaagtcagaaggacctcctgtgcctggctgtgcctaagggcaaactgacctctgagcctgtttcatctgaaaatggttataattcatttccttccaggggttacggggtggcttaaatgagataatgtacgtatataaaaatgacactacacggtgaaaactatcatctacctcctaggttagagaaaattcataaaaatataccttctttctccaaaatgagaaataactaaaggcagatgttttcagacctgcaaggtcaatgtttactcctgcaccatgtgaagtggcactgcagtggcctcaagtatagtgagctgggagctgcacctatgaagaccagagatgtcaatcatatcctgaaattaccatacctacctgcatatctacctgtctttcttcctctccccgcacccataattagtgggcaatactgagtatcatttttttactggttactgtgcgcctggcccttggctgagtgtcctgtgaaatgcattctctcattagcttttcccatcaccctgtgtgattgattggtactaccatacatagctcataagtggcaagatcagtgaaatccgtatctctttggcaggttcttaatctctgtattctttggtctcgtgttctttccccacaacttgtaaaaataaaatgtgtggaccacagtaatgtaggaatgaccttttacccataaattgtcccaactaacttttgtgttttaatttaaaaaaacttttggaaaattctacttccatttataaattaaattatgtgaatttcccaagcatcttcctaatgggttatcactttttgaccctgttcaattgtcaacactgtcttttccaagcagaactattcctggacatttctgacgatggtgaatgaggaaccttgctatctgagccctgctgctatgaggctggtgatgggttttgcagctgagcaccaattgcaatgtacagtgtctgtgctgcaaaactctaaaataggagcttgtaaattcgacaggatgtagaccaggaatcatccatcacgttagagtttatttctattccctgattgtgtatatcattttacctgataacttgcagtgattccttttgttttcaggggagggtgaagaaagctatataaattgtgctttagggatttggtttatcttggatcattaatgtatcatgtgctatgaagctatgatactctaaagttattccacagtaaggacacagaggaaatagcccaagtcagggggaaaaagtgaaggaactgcaggaaaagtgaatggttggctgataatgtgaaactcagattcagggtccccaaactgcttgcttattttagacaacatctcatattaacagcagtgctggcaaaataatatgtttatcagaattaatcaattgattgcttgtatattgaacagtctacacacatttgtgcagaaaaatacttctaacatagcatttggctggattttaaatgataggctagtcatgaagataaatgctgatgtttgtgcatttggactaaagaaaaccaacatggtttaatagaaagtaatagaattaatcaagtgttagaaccatggatctgaggcaatcctaatggcagtgctaacaaacaccatatgcttagagggctctggctttggagggagaatgaccctgagttcaaattctgcctctccctcctagtacccatgtgaccttgagtagtttatttagcatttatgagcctcagttgtcttgtatataaaataaggagaataataaaagtagtaatttgattctgagggttaaatgagttaatgcattaaaactcaccacagaataaacattcagtaagcataacctatttatttagtttcaaggaaatctggaatacaggaaactgaatttttctaaggaaatttgcattttatatttgtatctgttattttacctcataatatgagtcagtcagtgttgcagagaagtagcactatgccagggtgactagagagtggttttggtatatattatataaaaggaatgattacagaatttgcaagactcaaaaaatgcataatgtttttactggtagcttatagtaagtttgtgtgtgcatgtgtatgtatatagtgtgtacataaatatcatacatatgcatgtgaaaataaagccaaatcaaagacagtgacatatctcgaactcttccctagggaattggggaatcattgctacctattgtctcccctttcagagcttcgtgttgagtatatgtttgagaatttcttttaaaaattaaatggaatgactctgcgtgatctcatgaatagccaaatatattcctaacttttatttaagcccatctttagcaagtaattgctggaattatgttagaaaaggggaaagatcttgaaaggacatcacattgattaacttgaagctaatataccagagagataatggtaactaatctcttaaaaacacatagagcaaccttctgtaacctgccctgaagttttcagttcctttgatctcagttgatgatcatttaatctcatatcttgagaaggtgggggattagaacattaacttaccttaagtattctttaaccatcatcaaccctctcctaggcatttcctcctatagccatctcctaacatgtcaatcacctttctcctggagtcctgaggagcctcgtgaggtgtgtgtatttgccacctctgctctgccacttttctctgggccctgtgctttctagcccatcctcccaacatggtatttttctttccatccaagtaccatgttacactgttgccatgtactaccactgtaattccccccttgaagtacatttattctgttattagtttaatacatttggattcccttctgactgaaactgtagaagcctctagtggtaagttgtgtgatgatccccttcatcatacaatttaagcttgacaacattaactactgttcaactgcaaatatgttgcttacactgaacacttgaattgtgaacaaaatcttttaattcaaaatgtgctagcttttcacctttaccagctccccagcaggtaattggctacattactaagaaaaagacaccatcagacatttggtccttgaagttaagtccacagaatgatctttatggaggtgaagggtggaaaaggcactcttcctcataagcaactctagagaagtctagttcccgagaaattatactaagccacagttctactcagtgatcacgctggctcaagtccagccgtcctgtggaagcaccacagaggcttaactcttggaaagccagaaggtcatgccagctctgatttttcttaactatcccagtctctcattataatatcttatagaccagtatttattcaaaagtaacatgtatatgaatcacctgataatcttgttaaactacctggcaaactagatctggggaagggtctacaagtgtgcatttctaacagtttccagatgatgccaatggtgctggtctgtgaaccacactttgagtagcaagactgtcatctgctgactctcaatttcgtttgtacattggactcactgcagagctttagcatgactgatgcgtggacccacctcagataatccgatttagttggtatggggtgtggcccgagcaacaggcattgtgaaagctccaccggtgttccccatgcatggtggagtctgagaactcctgtcttatatgtcatcctcccagcttggagtcctccttatgtaccactataaccctacttttccataacgcctctctatcctttcctcattatttctccatcagaaaaccccactgactgcctgtacctcaacagttgaatgtggccacaaatctcaagtgggtgcaagcactcttggggtcatgaactcttcctattggaggttttgaagagctgaagggagagcaagcatatctctcccaattctgtacaaacctcctcaattactagggggaaaaaagcaaacaaatgaattctgtcatctttgcaccaccaaatccgtgcctcctacatctgtagccatagatgtctcttgcacctctcttgcaccctggttccgtgctccctcacctacccaaagaatcgactcctgaaaggacttctttcgctaagccaccagtttctcactctcctagattcttcccatcaccatcgtatctgctgtcacattcctgtcattagaagagcttcttgagcccattttcccctctggtgactgctctccttctctgctctttccagctttgccataacatgacacttgccaagtctgaggagccaccttgttgccaaatcaagtgttccttcttggcttatcttgctggggcctctcacatacattcagtggagcactcttgcattcttaaaatccttgtttcctttcccttccctggtcagggtccctcttctcacacacagactcctcacctctgggctctgtgtatgttcgctactattgcacctgttcatacctagctccctcacctttgtcaggcctctgtttctctctctgccattacctcctgaaagggggcttctctgaatttgctttaaattagccctttccactcatcctcatcacttctgtatctgcttgtcctggtttttctttcatagctcatcaccacctgaaatgcttatactcccttgttttcttgtctgtttatcagatatctcatccagtaataatataaaccacataaaggtacgggctttgcttttttggttatctgctctctgcttggaatttgaacgtgtctaatacataattagtcactcaataaatatatgatgaataaatgaatgttcctgaaaccattttccagttcctcctaagaacctgtaagcccactacctatccatcatagacaagtatcacaattgtaaaatttctgttctgatttgtgactgaatttcattttatctcttatgatttaaagagtatatgtattctaaaaaatccagtaactttttccaccttggttcatggagctggtctgccttctttcttccatgatgaacttgcatatcttctatctttgtgtgcacatgtctacacatatgctacgggtcccaaaagaataactgtttcaagaggttgatgtctttaaaaaacaaaaaaaaacgtccttagtttctttaaaaatacataattgctgcctctccagagatatttttacagtgtgtccctagaggaggaattccagctgatggcccaatgcagggcagaggcccgtagacagacagtctggctccaatttatgctctgtgttcacatggctaataggcgttagtattttgatttagagaactgctgtactggatcctgccctggttatacactggcaacatctggcttagaaccacacatttaaaggccaagagctatttattttcttcatcttcctcttctctctctctctctctctctctctctcactctctctctctctctattcagtaaaatgtctggggaatagtttcagtattttgatagagcagtacacagaataaattggcagtagttcaatttgcttcctattaaactctgttgtagacactttgaaccttttggaactgttataatatgtataatatatatcagaacttccacaaaaccctgttagtgcctttattatcccacttttttcctctatccaagattttgctatcacctcattttagaccctaaactcaacattcactacacttaaatgcaaattaaaaaaaagcaactcttgattcataaggtgcccataattctacaaacatctaatgaaaatgagcaaacatctctgtataaaacttgggttcaatccagaagtatgtttcattctcacatgcctgataaaacagtggaattgcaggtgctggcagctccctcacagcagtttggcaagatggagggtgcccagcattgctaaggctacacccaccaaatgtccagtcatctttctgggattcttggatctgtgagtcctttagctggaaaaatgcagtgctgtaatctgctcacttagaattagctgtcccagtgacttagtaagctggaaaaaaacaaaattaaaattagatatttgaaagtaagcctgccccagacattctcttagaaacaagatcaccacctcttacaggcatggttttccttggctgatggcatgactagccctggcccatctgtgccaagactttggcatggctgatgaaacctctctctttcaataggccttgctctctggaaagcttagtaaaatacagctagttcacactccagcaaaccacaaacctaaactttcccagttacaaatgatattttgccaaaatgcttactaagtcctggctctcacagagtgtgtgatgtggaatctccacagcttgaaaaacaaatgaccagccctcccagtgccgtggggttgcattccagtggagtgctcaactacagtttttatcacaccacatgccatgcttgcttgctagcagattcttgcattttgctttttttaaaaaaaattatgcttgtttgcatgcttatcagatgtacaaaatgcattcttcatggattttcctatcactgtttgggtagcacagagttacgtacttcagaagcatggaattgatcaaagtaacaatcaaaaggaacagtatgttttctcccatccccataaatctgatctgtaaatgcaaagaatgtgaagaatgaacataagaaattcaataatggtatttaaaacttaaaaagaaaaattggcaaatcataggataagagcctaaccaacctggtctccagaaggaaaaccttttaaatcagatatttctgaactcctatctgaaatatatatctttagggcagtaatcatcagaaacataacagactagtgtggagctcagttttattgtcttgcatcagtgaacattcctcagaagcacagagcgtcactaatggaattatatgttttcatgctataattacatttgatttttcctgatatttctgtagactttgttcatggttccatgactaggcagaatagtttcatagaatatcctcagcctagttaatgcctagttcccccaaccctgtcaaaaagacagagactaattataggaatttttcaaccaaaaaagacaaaatgttttttttttaatttacacaacctttagagattttatttcaacaaaaacatgatcaacaaaatgaagaaaaaagaattttatacttgaaggaaacagagcaaggggcccttctgagctctgaaaccccttgagacacaaactggcacttattaatattttttttcttttgagatactgtttcactctgtcacccaagctggagtacagtggcatgatcttggctcactgcaacctccacctccaaggttcaagggattctcgtgcctcagcatcctgagtagctgggattacaagcacacaccactgtgtccagctaatttttgtatttttagtagaaacagggttttaccatgttggccaggctggtctcgaactcctgacctcaaatggtctgcctgcctcagcctcccaaagtgctgggaacacaggcgtgagccactgtgcctggcctacttattaataattttattcttgtttttaatgaatatttgttggctaaaacagaactgatttgatctttgggtatatagtcccaaatggttctgtcactggaagacggatgaatccggcagcacagtcaagctaccggatgacataataccaggaggccatcagatatgtgaagagtcagcatccattactgagaaatgagaatctctgcctatgaaatccacataaatctctaaatgagaagatgcaaacaattagaaaattatgttgaaatccagcgtattattatcttgtctctatcttcactcacatcgtcttagtttagctctcataccccagtgaccccttagcactcaatgcctttttcttagcatttaattcctattctccaggttttcatggctatagaacacaggatgacatctcaaatatgtttacactttagggcttccccctcaaaacatgatgaaaggaaatataggaataaaatggattatcatagtggaggtgtggaatgaagagtggtttctccttgcaaatggaatcagttcagggtgtagatgaatactgcttggcctgacctgggttgcaggttcttatcataaattgagtaaagtttgcattacatttccccaagttgagatacatttcttgaaatgccaactattagaacgttgaaaacacaatttcggctctctcttctcaaatccctgcagtctggctcctggcccagccactcaacccaaatcaccctctgaggtcttccaaaataacttcccagccaaaaccaatagtatccttctagacttgtcagctttattcaaaaccttattcttctccctgctaacacccatctggtctcaaattttaagggtttgtcccttccaggttttttctttctcttatcttcggagttgcacctatgagcagttcttctttagacatcattccaactttgataattttaaacacaatgtttgagcatgaattacttccctattcccatccctatccccaaatgctcctttctttttagtcttatatctcttgatgctaggccttcagctgcagttaattatggtaaagtaacaactctggtcttgattttcgacttcttgctgctgtttaatatcttggtttctgtaaacaaaaattgagtcattttaactctttactgaccccatcacttcccaaagcatatattttttacctggattacacttagagaactaatgttcttttagctattctcacccctcccttcttcaaccttagtcaaatgcaggttagaccaaactctacacatgtttggatagggagattcctcttaccttgttgataaaaatgcaattaaaaattaagtgatggtcgcaccatcctctgccagctcttcctgtttcacctccagataacattctctactcaagtcctataaatacccagttgacctatttttctctatcttttaattcttattatttgtcattattcttaaaaagacatcctttctttttagtaagtttgctccattcttcagcaccccatccaaatacctttctccagaatactttccatagattatatgtaaactgggtcatatgaactagctgatttccttcctccttctctcctcaacatacagctttatgtgaaaatgtatgctgagtcatcgttacgtatgtctgaattcccaggtgtgtctgtgcatttggtatgtctgaaccttctggaaagtcacctttttggaaaactgagaaatcttttgaatcttcagtaaaaacccatgcagtcttctccctcttccaccacagcaatatgtgcttaaaatatttttaataataagcgcttactgaaattttacctcattatgtgtacattttctttacaggatccccccatggctgtaacccttggactccgcatggaagaaatgatttttaatcttgctgatactcatttgttttttaatgatttagaggtaagaattttacaaggtacaaatagttgtatccaacaccagacctcctagatggtttctgtatctgtagataacctcgagaaaacagaaagataaaaattcagctcaagagaagcataaagttcttgtcaagaggaaaaattgtatatacaaatataagcagaaagttaatttgtgttcatgaataatacatatgtagaacaaaaatgtctctgaagaggagattgttggctttgctgtccttactgtccacagctgtacatgactcttggttcttttagtgtttcagcattagctcttccagattattaacccggaacctatctaggttaatcactaaacgatattcacagacactattcatggtgatagcgtggatcaattaattttgtggataatctgaaacttaactttagctaacattatcattccccgtttctttcccttctggataaaacagtgctagaactatttaagcacatttgatgcatttaaatgcatttattttctcattttaaaaattatgtttctattttcaacctcaaatggcaagacagtgtctaaatggcaaataagaggaatagaaaataaaaatcaacagcaaaatgaaacgacccaaggaaaaatagcctcagagtagttgaatgtgtactgcaataaagtatgccatctgtgtgctaatgacctaggttattagacattcagataatagaattcgaaataactaaatagttcaggttcttggcagaagtacttctcagtcaagcataactatgctgaaatagagatttctctttcattttttttcagatggcatgtttaaatttattttttatctcatagtttcataattaaagtttttatctatagtcatattatgaattgagagggagattactactactgaacccctaccttgttctagatattttatgttggactttccattaattacttaacaatgtattgaagtaaatgtgtttatcacattatttatgcaagggagaaactgtatggtccacaacatacaaatcacatctaaagttacctgctatctaaatggcagaactggaatttgaagcagatttgcgtggctccagagcacatacttctgacccatcctttgctgaaaattttacaagtgtgtgagaaagaaaagccaagttattgagttacttaaataacttttttttttaatgctctaagttctggagtacatgcgcagaacgtgcaggtttgttacatagatatacacatgctgtggtggtttgctgcacccatcaacccgtcatctacattaggtatttctcctaatgttatccctctcctagccctccaccccatcacaggccccagtgtgtggtgttcccctccctgtgtccatgtgttctcattattcaactcccacttatgagtgagaacatgcattgtttggttttctgttcttatgttagtttgctgagaatgacgatttccagcttcatacatgtccctgcaaaggacatgaactcatcattttttatggctgcatagtattctatggtgtatatgtgccacattttctttatccagtctatcattgatgggcatttgggttggttccaagtctttgctattgaacattgctgcagtaaaaatacatgtgcatgtgtctttatagtagaatgattaataatcctttgggtatatacccagtagtggaattgctgggtcaaatggtatttctagttctagatgcttgaggagttgccacactgtcttccacaatggttgaactaatttacactcccaccaacacgccagttagaaagtttttttttttaaatgacaaggtcttaaatccagtttgagttatatttgagacatgagagtaaaaatgagtggttcagtacattccataaatgtgtgtgctgaaatctgtgtaatagcattttgctgttgttgttgttgtttttctctatggtgagtcatgtttttactgaataatacaattactgtctgtttaaagcggcattcttttttttctacagtggacagtatgaaagctcatattactaggcaagctttcagttttagataacaaataataatcacctctctaaaacctgatttaaattgatcctgctacttatgaatacagaatgattttatatatatatatctggtaataacaaacaccttaacctttccacaacttgaattttttacatacagcgataacttttggggttagtttttctgttgatcagtatggatgagttgcattgtaaagaggatcttaatattattagtcttttcaaaggtataaaaagaaaaaagaaattttatgtggtagtatccaagtaaaagtgcttatttcatttggttaattattttttactggaaataagtggaataaatgaacttttagcttttgtagtggcacattagaagccagatggaataaatttgctacccaacttttaaaagaacactgcagtgagaaggtgaaagtagcatataatgaatttgtaactaactagcaagtttgaaatgaatattggtatatcaaaaacatgttgtagtcatgtatggcacttaaagacatttattttagttcaggtactatgttatatgtatttcaaaaatacctggcaggatctttggtagaagtagttcatgagaaaatactggtattgactttctcttctctgagtatatagctttttagttgtgttttagaatgtgataagaatttcatttgactcaaaccctagaacttagctattcaacaatactgtacttaatcaataaaataattgagtttctgcagataaattgaactctcactgtatatgcgtgccacacaaatgcttgcacatccagataaagtgcccaaggaaacataaatgatttttactgacttgagtaaacaagcactaaaaccaatagaatcaaaacagtagctttcaaaagcattcagtacagttacatacatagcgccctatccttctgcatctgactttgaaaggcagttttactttcacacaactttttatttcaataaacctcttacagccaaaaattgtcttgggctccataactggaattaaatgatttctactcttgttgacaaaaagaattcctcaagagagtaactctaacattagcttattatgttgtcattgttgatgttgaatatttaagctctcagggtgcttttcttaaaaaaaaaaaaaaaaccgaaccatctaatttgacactccattctttcagtagatactgcaacatatatagtagcagctgttcctcacataattccttaaaagatacaaatttaaatcctcctaatagacacttcactgcatcctgcccaactgcagagaaggcagatgcccaaactaccctcccccaacagaagagaatcctgtgtgtttaaataaagtgctatttcggttagaataacctccagaaaagaagtctcaggaagtttgagatggcaaaggtgagaaaagactgctgtgaattataactgataaatatcagtgaccactttttggtcactttgtgagacaaaattaaagtatttaaagttagttctagaatattttcaaatgtttgagttttattaaaatttgttaaagccaaaacattacagtgtttttagtatttcactttttggtcaaaatgattcttacaaagtttctcagagtgaaaaataattgaaggacaattatttagctttttctaaaaatgtaaataatggaagttcaggtttctttcactggaccgtaatcctcaggcaaccgtccagagtcaacaggcatagttaactaggcataaatttaaagaaaagttaaatttggaaaaacctcaaatataacctcaattatggagcttatggtccaataagtgtgatatactgttgtcctcaggagtaaaagataagtttttgggcaacctatgtacaaattaatcatgcgagttaatatttaatgtcagaggaatttctctaaaaattacaatgagaggatattaaatagagaaataacttctgattaccagggaaatttacaaggattcctccacttttttgaatgtcattaatttattataaaagagaaacacagaagttatttacatgtggaaagatttcagagcttcaatggaatgggcaacttcatgtgacaccatttcaatagtgatttatttcagtctacggactttccaagtatgtcaccatctctaaataagaaataatctcatcatttagaactactttggtgcctccatattctgggagaaaaactttatttccaacccttatgctaatgggtaaaatctctccactcttttccttaaagcccagtccagcagttactatcattgcttgcaatgcctttactggatgtttttcaggaagcataatgcctcctttggttacagtttggactgcactcctttcagccaatacttggtcaaagaggggaagacattttctaaatacctgtccggccaggccattccttgggcagtctctgcactcttctctctcccttccttccactcttgacataaatatcttcttactctcctcaccatttagaatgcttttattttatatcatcgtaaccttgtgttagaaaatttaaatgattgtggtaatcctgtaaagaattagcatttgtctagacttctcccatttatttaagaaagaattgtggagtgattattctatgctagattgtgccgtaagtggggatgtaacctggtgaaccaaatgccgcactccagggcacttccagtcagattaaatttggggacccatacgtgtaggtggagatttattcaaaacttactaaccctgtaatttctgtaaattatatgaacttttaacttacctggattttcaagttacatactcagaaggtactgattgggctagtcactgagttttgtatattaaaggaagcctgagtcaactatacctttaaactggaaccggattaaacactttctgtgtggaaatctggagactaggtatgaaatatttgactgccagatactttcaagcccaataatgaatataaatagtagctttcaaaaaacctttgtttagtatggtgtattttcaaatttaatgattttttttaaaaattaattttgaatgctaagcactatgtatatagtcagaagctgtaagtatgagtaaaatactttgagggggactaaggaggcccaagatgctaaaccatacttactctgaggttaatatgatgtagttatttattataaataatttatttttatttattatatatttattattctgagaactaggcattttaaatgccctcaccaaatttcacctttagatcttgtaatattgcgctggaactttgcagattatttgaagatggactaagactcagaacagcaaagaaggatcaaatccttatccacataagttaccatttgttctgagcacacagtaacttacaatcaggtcaactgtggccgggcgtggtggctcacacctgtaatcccagcactttgggaggccgaaacaggcggatcacaaggtcaggagatcgagaccatcctggctaacacagtaaaacatcatctctagtaaaaatacaaaaaacacaaaaaaattagccaggcgtggtggcgggcgcctgtagtcccagctactcgggaggctgaggcaggagaatggcgtgaacccaggaggcagagcttgcagtgagccgagatcgcgccactgcactccaacctgggcgactaagtgagattccatataaaaaaaaaaaaaatcaggtcaattgtgtgggtatacttacataaagtttattttgcttggaattttgtagcacatttgagatgataaatcgtcattatttcccttttacttaaaagtagtctttttgatttgaaaatttaaaaacttgacaaaaagaaaatgtcaatatctatgatcattaccctttttctgtgtcaaaagactaggaccttgaaataacaccagcatggttctttcatggttctggtccagcaggaagtcatggaaattccacaggacaccataatctgaaggcaagcccctctgttgccactcaacaaatgataaataatagtaaccatctgaggtgttcagactaagttttgtgtatcttcagattagccatgcttaagtatgcacaacagaaatggcctggaagggaactcaaatgcttatgaagattcaagagaatttcttatcaataattttaagattaaaatttttaaattgctctaaagaaactctcatgccgtatattttctcacccctcctttctatggaactcttctggatttaaattttgtggggtagaaatggtgccatttcccaacagaagaaaacaagagtgaggcaatgagatggacgggcacatgctgccatcttctggtggtagtctgatcagatgagaagatgggaaaaccttcaagcatggtaacaagcatcatttagaaaacaaataacttttccaatgtttttaacaggagtgtgatcaagttcatatagatgatgtttcctctgatgataatgggcaggacttaaggtaagctatgcctttcagtatgctgtttcctacagaaattcggctgtggagttttgcacatgtaattatttctttttaatgtattttgggtgttacagtacctacagttttgcaactgatggcttccatgcagctgcaagtagtgcaaacctttgtttgccaacaggtgtaagaggaggggttgactggatgaggaagttggcttttcgttacagaagagtaaaagaattatataacacctacaagaacaacgttggaggtatgtgtggctttttcaatctaacaaaggtactctgggtataggtagaattcaatctgtagtttcatgttaaagatgattctgctgtaaaagaaacaagcaaaagctcatattaaaccacattgagccagaagtttcttagaattggtgggaaaaaatttaactacaaaataaaacatgcaccaccgtcacataaattgggaattctttgtgtaaccttgtgcccagatgtttggtcaagatttataccagggcatttagttgaccctttatcagctggtgcttggagcattatcagtgaaaatcagacagaggaactaaccgaggcaacagaaagaagtcctttctcaagtagagagcctcaatcattgaaggacggtggttagcaagcattatctttctggaatcccatcaaaaataaatataaacagttattaatgtcatttcaaagactttcaacaggtttattttatcttttaaaaaaatcatgttttatgtcagtgtatcattctcatgcatagtgtcaactatgattatcatagtttttaaaagttcaaataatctgtaggtaatatcaaaaccattttataccccttcatatattttacaggtgatatcaaaacctttgctatcaaaactgtgatcctggatgtgcttctatatataaaataaaccttggtgatatcttattagaatcaattttaggtaaacaagattcctaaaatttgctattaggtcacatcagattaatctcccaggattttgtttctttatgtgcttctctttttttctaaaaaggaagagtaattgaggaacataaactagaatccaatatactaaatatacatgtgcatactatacatacatatattcacatatatatcatagaatacaatatagtaaataaaatgttcatatatagtatataatcacatatatccatatacatcatgagggtggggctatgtgtctggtcaccttgtatccccagtgcctagcagtgaaacacataagagatgctcaacagatacataataactgtaagagaacaaaaaatgtagagccaaagaggataataatgttagcaacaaaagggagcttagatatggaagacattcatttccaaaaagaaattgacccctgcaagctgccgacttgcatggggtgctggggagaatctctgatttcctgatttgctctagtgctctgtacatgatgcttttgtaaaatcaaataagtagattttctagttaaaatcaatggtagataatatgttttaactttaaaataaatatccccttgggaaatctgagttaatttttatcattcagatgtcatcatttccttcttgcggtgtattaataaaattattttaagttaacaagaagactaagaagtaatcttgtatcttagaaactttaaacaaccacgagtctactttctgtcatacgtgagcatctctctattataatgaggacaaatgagacttgctagaaactgtaaattttgattgaaaaaaccatacaagtgtgtatgtgctcccgatattctaaaacacaatttcaataagaaaccttcctttttaatggatttacatgatcttgttttatcataaaagcaagaactggatcttttaaaataaaaaattagcacatctatcttttttcttttaacatcaatattataattcatgattgtaactgtcctacacttcatgattttaaacaaaattattaattttttacatagcaaagtatttatagtagcattagcagtagtattagaaacattttaacagctacatatgatgatgccattgttactgtttgaaagtggaaatgattgtgattattaagaagctaaacgttttcagacagtcaaaacatttgggatttgtgtccctaggttggtgaagcccaactattcctccagacctcaaatatcacagtcccttggaataagatccaggcaggggcatgttcaaagctctttagatgattgagatccactgcgctagattgtaggatcagtgagggcagagactggggccacttttcagtggcactacctgagacctagaagttcagtaaatattcattgacggaatcaatggatattcaattcagaaacgaaggatcaatatggacataaatctgtatctctctgcatttctgatatatactgaaataaaaatgatctattccagacagaagggaatctaaactagatattctgagacaatattcatctctcgactctgccttggttttttggtgttgcaggactccttggccctgccaagagggatgcctggctacagttaagggcagagattgaaggtctgacagattcctggctaacaaatgcacttaagtctttatcaattattagcactaggtaagtggaattgttacctttctatgtgtatcgtattgaagatttagttctaattgtacaacatagaaaaaatgttttaattcctagactgttttagactttaagggacctaacaattctgagttcagtgattttttcaatttcagtgcatgtgcatgagagagagagagagagagtgtgtgtgtgagtgtgtgtgtgtgtgtgtgtgtgtgtgtgtgtgttgggatgagagaaggttgctgcttctcagaataaacaggagaccctatctcaccacccatctcttctcatagcaactgataatccccacagaatggttacatgacttctaatgaaaaaagaaagagaactcagttggtggcaaagtacaagggggtgatggcaaatatgaaacagtactgttgatacaaaaaggcatgttttaggtgttaggccataatggataatacacataatgtaagcacatcagaaaagcaggagaaagctcttgaaaaaattctaaccattttgttgtttgaactaaacagtgaaattctgacttgtgtcagtatacagagcagaattaaattgatttgtaaccagttgtacaaccaggtgccaaatatgctaattcataaagtgatgtaagtaagtctggaacgtgatagctggtaaggtagagttgaatctctgtactgtccgcttccattattttatatctgagttaggtgaataaataaaagcatgcttaataagtgggagctaaatgatgagaactcatggacacatatagggaaacaacagacactggggcctacttgagggtgaagggtaggaggagggagagaatcagaaagagtaactgttaggtactagacttagtacctgagtgataaaataatctgtatagcaaaaccccgtgacctatatagcaaacatgcacatgtatccctgcacctaaaataaaagtcaaaaaaaaagaatctgtcagcatttccattaagaaaagattaatgggtcaggcgcagtggctcatgcctataatcccagcactttgggaggccgaggcaggtggatcacgaggtcaggagattgagaccaccctagccaacatggtgaaaccccatctctactaaaaatacaaaaaaaaaaaaacaaaattagctggtcgtggtggtgcacacctgtagtcccagctactcgggaggctgaggcaggagagttgcttgaacctgggaggcggaggttacactgagctgagatcatgctactgtactccagcctggagacagagcaagactctgtctcaaaaaaaaaaaaaagtaaagaaaagattaatgagaacgtgcagtatttgcttttttgttcctgtgttagtttgttaaggataatggcctccagctccatccatgtcctggcaaaggacatgatctcattcttctttatggctgcatagtattccatggtgtacatgtaccacattttctttatccagtctatcattgatgcacagttaggttgactccatgtctttgctattgtgaatagtgctgcagtgaacatgtgtgtgcatgtgtctttattataaaatgatttatatgcctttgggtatatacccagtactgggattgctgggttaaatagtagttttgtctttagatctttgaggattcgccacactgccttccacaatggctgaactaatttacactcccaccaaatgccgcatgttctcacttacaagtgagagctaaatgatgagaacatgtggacacatagaggggaacaacacacactggggccttccagagggtggagaatagaaggagggagaggatcagcaaaaagaaaaatgggtattaggcttaatacctggatgatgaaataatctgtacaacaaccccccatgacacaagttaacctatgtaaccaacctgcccttgtacccctgaacttaaaataaaagttggaaaagaaaagatcaatgaataagatgacaaattgttagttttaaaaaaagaagaaaaaagcatgcttaataaatgtatggggaacacagcattcagagaaagatttatttggactaattgaatcagaatgcagaaaagcctgacagggcaaaaaaaaaaaaagttaatattaagaacatgaaagtaaatgaatagttgtcacatcctgcacagcttctgactcgtggcacaggcagagctgcatggatagcagaaagtccaaatggagagcggaggggaagtagcttcctgtgtctgactggggaagccatggcagcttagtttgtcagttctagggaccatattccagtgggggctgtggcagggttgaagcagcctgtgtcaagtgtggcagtcacgggtttggactgtgggaaccacctaaaagaaacagatggaaggaggagccagagagcttagtcagaagagtcatgtgcagaaccaagattaccaatttcacctgtttcagacgttaggctgaaaatcaattagatttctccctgtgtctggaaaggaaagaactgggaccaatgagtaggaatcctgcaagggagattctggctctatataaggaagaaatgtctaaaccagttgaaatgggacttgctatctcatgtcctctgtaattggtgacaagagagaccaaatgtcagggacgctggagcacgaggacccctgagccactgacatgatgtgtaaaatgtagtggagtgtgcatttctctgagaaggaggttgatagctttcattatttctaaaagatgtcttctatataagttaaaaaagatgattacattattagattatatatactatataatatatacagattaattattaaacagatggctttaaggcaactaaaggaatatgtgatattccagaaataaataaagatataacaaggaagggaggagaaaaagaaatcaacatacattgagtagttacatgctatggaatttgctgagttatccacatatattaccatattgatacaattgcagttttgccattacttttaatggcaaaaaacaaaattgcttttgcaccaacccaatagttttcacaaaatgcctggaatgtcagaaaaacagatgctttgttgaattatgtagtagaacccctagcaaaattaacttaccctctgaccctaattattctgtgaatatagtctcagaataacgaaagatgtaagaatatattttggtagcattacttgtaatatttagaaatcaaaagccacttaagcaactcaatttaactctaaaaaattctggtgcatcccacaccacacctattccaaaattgaccacatacttggaagtaaagctctcctcagcaaatgtaaaagaacagaaattataacagactatctctcagaccacagtgcaatcaaactagaactcaggattaagaatctcactcgaaaccgctcaactacatggaaactgaacaacctgctcctgaatgactactgggtacataacgaaatgaaggcagaaataaagatgttctttgaaaccaacgagaacaaagacacaacataccagaatctctgggacgcattcaaagcagtgtgtagagggaaatttatagcactaaatgcccacaagagaaagcaggaaagatccaaaattgacaccctaacatcgcaattaaaagaactagaaaagcaagagcaaacacattcaaaagctagcagaaggcaagaaataactaaaatcagagcagaactgaaggaaatagagacacaaaaaaaccttcaaaaaattaatgaatccaggagctggttttttgaaaggatcaacaaaattgatagaccgctagcaagactaataaagaaaaaaagagagaagaatcaaatagacgcaataaaaaatgataaaggggatatcaccaccaatcccacagaaatacaaactaccatcagaatactacaaacacctctacgcaaataaactagaaaatctagaagaaatggatacattcctcgacacatacactctcccaagactaaaccaggaagaagttgaatctctgaatagaccaataacaggatctgaaattgtggcaataatccatagcttaccaaccaaaaagagtccaggaccagatggattcacagccgaattctaccagaggtacaaggaggaactggtaccattccttctgaaactattccaatcaatagaaaaagagggaatcctctctaactcattttatgaggccagcatcattctgataccaaagccaggcagagacacaacaaaaaaagagaattttagaccaatatccttgatgaacattgatgcaaaaatcctcaataaaatactggcaaaacgaatccagcagcacatcaaaaagcttatccaccatgatcaaatgggcttcatccctgggatgcaaggctggttcaatatatgcaaatcaataaatgtaatccagcatataaacagagccaaagacaaaaaccacatgattatctcaatagatgcagaaaaagcctttgacaaaattcaacaacccttcatgctaaaaactctcaattgatgggacgtatttcaaaataataagagctatctatgacaaacccacagccaatatcatactgaatgggcaaaaactggaagcgttccctttgaaaactggcacaagacagggatgccctctctcaccactcctattcaacatagtgttggaagttctggccagggcaattaggcaggagaaagaaagaaagggtattcaattaggaaaagaggaagtcaaattgtccctctttgcagacgacatgattgtatatctagaaaaccccattgtctcagcccaaaatctccttaagctgataagcaacttcagcaaagtctcaggatacaaaatcaatgtacaaaaatcacaagcattcttatacaccaacaacagacaaacagagagccaaatcatgagtgaactcccattcacaattgcttcaaagagaataaaatacctaggaatccaacttacaagggatgtgaaggacctcttcaaggagaactacaaaacactgctcaaggaaataaaagaggatacaaacaaatggaagaacattccatgctcatgggtaggaagaatcaatatcgtgaaaatggccatactgcccaaggtaatttacagattcaatgccatccccatcaagctaccaatgcctttcttcacagaattggaaaaaactactttaaagttcatatggaaccaaaaaagagcccgcatcgccaagtcaatcctaagccaaaagaacaaagctggaggcatcacactacctgacttcaaactatactacaaggctacagtaaccaaaacagcgatatagatcaaaggaacagaacagcgccctcagaaataacaccgcctatctacaactatctgatctttgacaaacctgagaaaaacaagcaatggggaaaggattccctatttaataaatggtgctgggacaactggctagccaaatgtagaaagctgaaactggatcccttccttacaccttatacaaaaatcaattcaagatggattaaagacttaaatgttagacctaaaactataaaaaccctagaagaaaacctaggcattaccattcaggacataggcatgggcaaggacttcatgtctaaaacaccaaaagcaatggcaacaaaagacaaaattgacaaatgggatctaattaaactaaagagcttctgcacagcaaaagaaactaccatcagagtgaacaggcaactgacaaaatgggagaaaattttcgcaacctactcatctgacaaagggctgatatccagaatctacaatgaactcaaacaaatttacaagaaaaaaacaaacaaccccatcaaaaagtgggcgaaggacatgaacagacacttctctaaagaagacatttatgcagccaaaaaacacatgaaaaaatgctcatcatcactggccatcagagaaatgcaaatcaaaaccacaatgagataccatctcacaccagttagaatggcaatcattaaaaagtcaggaaacaacaggtgctggagaggatgtggagaaataggaacacttttacactgttagtgggactgtaaactagttcaaccattgtggaagtcagtgtggcgattcctcagggatctagaactggaaataccatttgacccagccatcccattactgggtatatacccaaaggactataaatcatgctgctataaagacacatgcacacgtatgtttattgcggcattattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaatcatcattctcagtaaactatcgcaagaacaaaaaaccaaacactgcatattctcactcataggtggaaattgaacaatgagatcacatggacacaggaaggggaatatcacactctggggactgttgtggggtggggggaggggggagggatagcattgggagatatacctaatgctagatgacgagttagtgggtgcagcgcaccagcatggcacatgtatgcatatgtaactaacctgcacaatgtgcacatgtaccctaaaacttaaagtataataataaaaaaaaaaaggaaaaaaaaaaattctggtgcatctacatggtagagtagtttttttttttttttaattatactttaagttctagggtacatgtgcacaacgtgcaggcttgttacatatgtatacgtgtgccatgttggtgtgctatttgtttatatctattaactattattcccacctcccaacaaaacaatgtataaaaactactctaccgtgtagagtatatacatgtataaaactactctaccgtgtatgtactctacattgttttgttgggagaggggaataatagttaatagatataaacaagtaggctcatgatataaatacataaagtagaatacaaaattgaatatacagaatgttcccaatgggaaaacaaaatctgtatatgaacagaaacactgaaagtaaatgcatgaaatggagaaagcagttttctccagatggttaaataatagttaatgttttttgttttcttctttatatttgtcttttacaaattttcctgttacaagtatatacagtgtttgtaatttaaaaagaaatgtaactctgaatatcacacagtgcaagtcacaaaagttcatatccaaaccacatctcgaaactttaaaagcctgtgctttatttcattctaccatgttttcattctgtaacattttgtaacatttgcctacgtatgacttttaatagcatattgttttgacgaaatattttgtagaactttaagcatcacagtatagtcatgggtttgtatattcttcttaataatctgccattattggaggagtgtctgtctgctgataggcaatagtaatgaattttgaaagggaccattaattaaggaatttagagaccacagtgacaattttaaatctattagaagttatttagtattagaaaacaaacatgtatatttcctccctattttaggagtaactgcataaatgtcttggtaacgacaactcaactgatcccagcacttgcgaaggttctactctatagtttaggaggtgctttccccattgagaatatttacagtgcaactaaaataggtaaggaaattattttaaactctgtatggaatgtgtccacatctgtgtgtctctgcctagttccctttttcagcaatttcacttagtacatgaaacaaatttggataaagttcaaattacaaagtcccctacaactcattgtacatgtatgaattttttgagaactgttgcctagtgacattaagaactcagtatctgagtatagatatttcattcttgtatttcacagaatattacagcccatgttatctaaataatatgtaatacctctagccatagtatttaatagtttaattagtttggcttataatgttgcatccttgcatatgcagtaatctagatccaagataaaaatacaattttggctttgcaggggattctttgatggattgaacgagtgcccttaattacagaaaaaaattaagagttttatgtatcagaaaacataataggaatattcataacacatgagcgactggaaatctgcatttccaaaaatgtgcagtagcaatcaaagaaccacaaccttgtttatttagatgatatctgcttgtggagccatctttgatttttaagtgcatttctcgcagactccaacttctaataaacattcaaaagcaaaacttcatcaacttctgccttattgaaagacttaaaactgatgtattccactgtctgtcaaggaaatacttgggaaaataaatgggttgtgtaatatgccttgaaggtcaacaaacttaggctctggcacattgacaagagaagttcaaaatcctaaaatcccccaccaaaaataacctgccctgggctttgaggaggtctgatgttggccctggcaaaaggagcctccagtggcagctgtttcaagagtacagttaaggagagccaagcttccgtagagtaaatttcacattgtcataagcactacagttcagagcctggatatcaataccatacatttttaatctaaatgtgtgtatttatgtcagcaggactaatagaaagaaggtaattttgcttttttgatgaatcaaataattttacagcagaataaacatttattttagtaaagacaatattctttctgttggcgaaatagttccttatattctttaatctgaaacaaaatcatttttattttttccattaagctagtattttttggacttctgaaccatcagattaagaaaaaaggcagaagtagccatggctgcatgaacattctgggagtgaaatttagagaataatgccaatgttttaaaaacaaagaatatgtagaaattccacggtgatgttcccgtagtgcaataagaggataccagtgttgtcagtactgtggcccagtcagacagcacccttagcagaagttatttgagcaggctaactctcacccacttgaagaaaggtgttgtttatcaagataaattcacttcttagtatttcctctttttttagcggtgaaccttcagtttttctctagaacacatttagctgacttgtctatgctacttaaaattgatttacagaattaaacaaaggttaaaacttatagttgcttgtaaagttaccccaatactctgttggtggagaaaaacatactaatattttctctaggtcaaatgcttatccctactgatgtttttggagtgtgaaacacatttgtttaatcatctgtccgaagtaacgccttaccttattcaaatatatgttgatttcataaacccagaggttgtgagccagttcaagtgatgttcaagaggcattaacttaacactttatgaatctttaggttaatgaatgcagtggctggaagaataagcagtgcattgtttttcaggcaaggaaagctgttttgagcgtatagtgtccagatttggcactaacataacttatgttgtgattggagatggccgagatgaggagcatgccgctaaccaggtaacttcactctgaactttatctcatttatcttccaggaaaagaaagttgctttgaacgaataatgcaaaggtttggcagaaaagtagtgtatgttgtaattggggatggtgtagaagaagaacaggcagcaaaaaaggtaacctgtctcaaacaatgtcggtgtgatacttctaatgcaagcctttttgtttgcatttctgtagtttggcccaatggcagcttgacaaatgatttaaatttatagatgcaaagcaataagagcatgagagggtcaatacttacattcaaataaaccatttggagtttagcattttttgcctgaaatttgataggaaatgcagggaaatttagttatatttatttatgtgtttgaaagtatatacatatagtaaatatatatagtaacatatatatttgtgtgggcatgtatacacatgcttatatagatgcatatgtatatattaatacacatacaatgtgcacagatatagatgagccaggggatctgcaagaaagattgggggggagaataaccattgttaagcacatgaaatttagcaagatagagattaatgagctccatacaaaagtcatgtgagtagttcatgagacctgcatgccatatggtcctcgtgagtgcctggaagtaggcatgctccatgataaggaatgctgagggcagatttaatagctgaagaaaaactgctacaagagattttaataagtgctactgaaatgggatgtattgctcagatccttttgcaccaacttgtcttagccgtaatgtgcctcacatctcctagaagagtttcatctgttttggtgagccagtggtacttgattgaagccaagcagctggctttgaacacattcagaagatcccatgggccctaagtattttaataagccttttcatctggcccgtatttctgaagaagaaaccaaggcaattaaaaaccattaaatggttggttcaacatcaaaagaagaatgatatcaggattggcccccaccaccagactgtcaactccaccctaagatctactgctgccttcaccgaatgatagaaaaatatcatctacccaagataaaagtgatagaatttacaaaacaaagccagatgatgaattagtatttttaaaacaatgacatgccagcctatactgttttacatagaaagaatgtcatctgtatcaccctaaggagattttaaatcatcatctaataaagcctttgccatttgcatgtgctggaatgtttacctgttttcaaaaaacagtcaacatagacagttttatagaaaagtttatggctataagagaaagagaaaggaaatgatgtatgtttctagtcgatttaaatcaagcaaaatggagaaaaggaaagcacttccactttagaagttttggtgacgtattggcagccatacttgcttttcgattacatttaagagagtaggctcaggctctgcctgccacagaacacctcagccttcattagcagtgtcaatgacccaagacagcacactagccccaagcacgtcctgtatgaggtcagggctgcagcctcaagaaatacggctttcattgctgactactacttcaaatgcttccagtcacactgaactgaaatatcttctggagtttgtcaatgtcctgagtccatgttgtgatttttcttattggcaccatagttatacacaacaaagagcagccaatgattagtaatacagccccactcctgctaaatttacatgtcttttagcctatgcaaattaaattcacgtatatttaggtaaggactaatataattcagttttccaaaccagtaacacaatatgcttttgacaattaaaacttagccgagtctacctgttttccacagcattaatgtttagattcggtttaccagttgttatacctaaaacttcaggagggaaacgcataaccctgaatggaaaacaatctcagatagaatgctatttccaaatggttgtttatgtaggtacagttttggtgatggtgaaggcttattattctattttaaaacagaggtgattggtttctctagtacccagaatgcatgcttttctttggatgtagcggtaatcgcatgtgcagaaatgttaagtactgttttgttgttacaaatagatctgagctgaatgaaaaaatgttacttctatgtgaatttcagtgacttaatttgacctagacatattttgtctgagaaatttaagaagaaatatcaaagtttaggaataggaatgtctagctgcgctcttccatagaaacatgtaatattaatacaagctgtatatgtagtttgaaattttctagaagctacattttacaaaattaaagagaaacaggtagaattaattttaataacatgtcaacccagtatacccaaaataattgtcttgatatctaattaatgtaaaaattattgaagtcatgtttacattcttttttgtttgtttgttttgtcctaagtctttgaaattcattgtgtattttaaactggaccaaccacatttcaagtgctcaatagccacatgtggctggtggctaccatagtgtgcagcaaggaattatatagcaataaggattattatgctacttagttcttaaataaaatatcttttgcatgttttatatctatatactcatttcccttatatacatgaatattttatacgtacatattctagaagatgtcatcagcccaaacttaacaaatcatgcttttgttttgtcagttcctgtaatgcagtgttttttctttttcattatcatcgccagtggggactctttagacagtttttttctaattactccctttcatgaaattttaatgtcacatatgtactgtatactattatagtatatcttttgaaggccacaaaccactgtaatgtctaagattttctgccccacaagagctcatttgtgtccctttggagatatcttccccattgaaaatgtatgctagagtactttctctcaattctacgccagtgaaataaatcaggtcgactctactggcttaagttattggttgtcccagacaagtaagaaagttataatccccaccttttttttctactttatgtagtcctgcttcaaattatttttatacagaaaggaattcaccctcttttcttcctagattaacaaaattaagaagtttgtcattttgtttcattttatttttaattgagaggcccgtaggagtgatgcactgattaaaacttgctttccattagccaggtaggattttgatgcccacttgctttcccagatgttgatgttggtagcgatacttccagaataaatattaaactatctaagaaatgttaatatttgctattattctcattaaaatccagttcagatctatttctctgttactttgacagtgccatattggtgctgccaagccttcacatatgatcatcccgcctttaaactctctcccctgaacttgggtggaccacacatccctgtgtgtgctgctgcttcattgagacaatggttgtgatctctctccatgcctcattccttccccttctctctcccatccctccttctcctaaccacacagcacaacatgcccttctggaggatatccagtcactcagacctcctggctctccaccaagcactggaattagagtatttgtaactgtgttctttagccggagatccattttttatatttcaagtacactgaatttttatgtgtgattcaatgcctctggctctacacatataaattgtcttaatggatgaaatcatatttggaataaaaattccagaatgaagaattcagattgctgaatggagttaaactttagtgctacagaaaagaaactctatggtcttatatttacaacactttaatgggttttttaaaaatctgtggaggttgctggtacacaccaaatgagtccaaactggaatgagcagctttagcaaagaactcttaccctggcaaagcagcaacacacatgctccgtctgacaaggtggtcaacaacattcctcaaaatgggagatcttctcagccctgaggtttgaatctgactttagcctacctaacccagaaaatctgaattggaatgcactcagactgtataaggacagtcctatttagacatgtaatttgtgtaaattattgatgaaaataatttactgtgactttattagcagctgactttcaaagtggatgcaatttttctttcttttgttggggaggggaatgggaggggaaatgggaatataatattgtctcttttttaagtttggcaaacagaatgttcatactgatgtgttgtgccttaaagacaagacagcatttgtgtgttacaatgtaactttggttaaaatctctgtagataatgaaaaaaaacaaaaaaaaaaacctttgtgatgattcttaacatgaccaaatttaaaagtcaagctctcagagcttaattaccgcatcagcaagaaactgagtattttttgcaataagaaaacaacaataataaaggaaagcttgtgtttcatttgggttcttaataattccaataattgtatgaggcaactatttgcgcatccaaccatgagtggaaggtttgggaaagactgtgggacctttacttagaaagtgaaatgtatgtagaagtctcaagtaccccttctacagttttactggagaaaactaagagccatattcatgacaacttgcacagttttgaggttgagacttttgatatgtgtaagttgcatagaggaggatattatcatgcaaatcatgagcaattatcacataaacttttttagaatgtgccatgaacatggcataaaattcacattgagtgcacagggcttaaaataaagctaagtatgtttattcccaatgccatggcaaaaatgataatatcatcagaaatggaaggcagttctcccagatggtgtctaatgaaagcaatgagtctatgaaaattttacctagaatatcatcataaattaaattagcaagtgcgctggatcttggcagcgctgctgaaatgacaacagtaaaataatacctggttctccatctgaatacatcaatgcaggttctcctcgtaacagacttgcatatgtttgttagtttctgcctgtattgtcactgcgcaacggatggcattcattacaagaagagcccatcatcgttgtgtttgcatggtttttttccttgtgtgtagcccatgttgggaacacgatacaggttctcctcttatttcctatgacacgatttcccttgtggaaatttaagactttaagaactagagtatttttatggtgtctgcacctgcagttctgtgtttaaaatgtcataatgtggatcctggagtcaggctactagtcagtgcccctagccagaggctggttcatgagttcatcaactgaggcctctgtggcttcaataaagtctacattttgctcacagatcacaacattcactgtggaaatatgatttcatttctttaggctacaaacctgtatttctttactgaatgctaaggccatgtttatattgggtagaaagatattgagatcccaattttgtacaagattgtgatttcattatctaaaccttaaacttaatcctttaaattttgtagcttttggctgcatctgccccaagtactattccaggcaaattaaagttggaatacctttaataatataaaaataatgatagtaaatcttatacttctgttggcccttagcttgaaaatagcagttaaaaaaatttaaatgttgccttgattatcagtacttaattatgttgtgcactaaaaccttaaatatttattactgtgaataaaaacaaattatctttactgtatagctggtttctttaaatgttgatagaattgtggcattacatctaaatttgtaagtcttttcatatcaaacaagcaaggctttttatgctgctaagtctgtgggtgcagaaagaaacaccccttggaagggcaaagagaagccggctggttgcatcaccccgtgcagtttctcacacacatctctttttctgattctgtgttcagaagaggctgccggcataaaacctaaatgcaaggttgacggagaacagcttgtctggcacaacaatggtgcaggcccacgagccagcatcacagcttggccatgggacgttgagtatgcacaaactagaactcttcccttcccaccttaggaatagaaaatcctcttcctttctaatctgaaaaacgaaaactgaacaaacacaaaaccaaccccttggcagttcccacctcctattgacatatggaatattgtgccttattgtaaaccagtttgaaaaatgttctgtaactaaagtgggttttcggctattatgtatacagcttggtatattactacgaaagataaccaccttgtgttgcaccttaaaaatatcaagaccatgtaatttccataacaaaataggtggcctggctatggtattatagcatacaattaggacactatgcccctgcaaaattttgtaaatcaaattcagaggcaaaaacatattttagaatcatacagtttgcacacgacaagtaggtaataactgtctctaaaaatgttttctccttagtccgcaatgagctaagattcagaatagtgtcaacagcatgctcctatatgaagttgttttttttaaagcacatttgtttatgacaagcctacattctcagtgaatatggcatttagtatttcttttgaaaacaaacttaagcatcatgagccaaagttgcactttgactcccaactacggtagcattatggacatctcacaatgtcaagggtttctgttatgtattagtaaagtatagattattggggcctacataatttaaaagaaatgtaaattaatattaaaagcttgtaaaaatatgtatatctttactatttctcaataaatacctttgcaattgttttcatttccttcaccctcccatgttgtagagtttttgtgtgtttatttagcttggttctaaatcatgtgaaatttgtatttaggtttctggtcttcataacttttcttttttatgcctaagttgcataatttccagttaaagcaaaatgagatgtaacacagtaaaacgtcactaattatgactaaaagtgagctgctttactgaaaagatgtcagttgcataattttttatacacaggtagctacattcaagtaagtataatgcataatgtagccacaattcactaatgataaagtaattaaaatatttagacctattagaatatttaaagcaagtatcgtttacgtgaatatgggacatagtttataagtatcttccaagaggataaaaaggtagttttctgtaacagatttaatagctcctttattgtctggtgtatagtatttatttatttgtgaatctgttgttcacatgtaaaccaaatgcaaatttcagataaacctctgctcattcacaacctttaaaaatgttagcctccaaattaatggtatttaagtggagctatgtggcaacattctagtaagccttacaccatccaaaaatcaatactgatttacagg

SEQ ID NO: 12 - DNA, 13711 bp, Homo sapiens
  source 1..13711: mol_type = other DNA
ccgagagccaggccgcctacaggcgtctccagccctaccccatcgccggctacagagcgggagccccgagccccagcgccgcgccgcgcggggagagaagcagctgttcccaacagctggggggcggcggcgcccggcgccccaggtgctgccgcggccaatcgcaggcgtccggcccgcccccctccccagccgggcgtcgacccccggcccgcgggggaagggagcagagaggtggagagctgtcctcggcactcgtcttccagatcccacggcgggagggacagaccagagccgaagctgcgcggagttcctcgcccctggccccgccgccgcgccccccgcccgcttcggcgctcggacgctcttcccgcggaccctccccacaccccacccaaggcacctgattcctaaggaagaaaaatctacctctaaagcacccgcttcttcgcttgagaactaccctctcttttcatttttggcctccccgccgggtgagtggaaaccgcttgcacattctatagaacccgattctaatccagacgatccggagaatttgtacggtgaccggcagggggggcgggaggagagagcgaagtcaagtctccagggccgctggcgacttcggaagccgcgcgcccggcgctctcggccgcccccggccgctcgcccccgccgcgccgccgccctcgcccccgggctcgcccttggcccccggcggccgcgaaagggtgcgggagacgcggagccggaggaggaggcgcagccgctgcccgagccgcagccgcagccggagcccgagccgcggggcgggtgcgaagatgcacacgacccagaaggacacgacgtacaccaagatcttcgtcggggggctgccctaccacaccaccgacgccagcctgcgcaagtacttcgaggtcttcggcgagatcgaggaggcggtggtcatcaccgaccggcagacgggcaagtcccggggctatggatttgtaagttgcacggactgggggtgacggggagggacggagtggcggctgaccccggggatcgggagcttggagtgaggggctcggcgtgacccgtgaggagccccgcgggtagagcggcgctgccccttcgctccggggtgaactgaaactttgctaggggagagggtcggcgccagcctcgcggggttcggagaagacccagcgctgtgcgaggtcgggggccgggcagggcagagcaggggtgaaaggagagacctgtaatgacggcgggatttggggtgcggagggttgcgagggaggggccgcaaccctgaacaattgcattcccgggctggatgccggttgttaagcgcgctgttgctttgtaaaaatgcgtgtgttctggtgttttggctggggcagggaggggacaccccacgaggtctggggctgttgcccttgtgtcatccccccgccttttaactgcaaaggtaggttcttttaaatcatgatcctagggctctttgccgaactgaaacagagcatattaagcttccttcctagagatttaagcgtgcaaattcagctgccgaagagcagggagccccagagtaggacaatagctattgtcgttggggagtgttttcgaggttgggagcgttgggcggatgtaaggctggactttctttaaagcaaacggtgcctctgaggctgagtagggacctggggggcgggggattcgggggaaggaggatctagagttccaagaatttaggcttccaaatctcggctaggccctactaacacccttaagatttcggcggtggggtggggggaggcagggttagagggtttctttcttttttccttcgtctacccgccccccccccccacgcccccgcccgcctgttaaaaatgggagatcaaggaggagggtccagccaaaagaagcaaagaatagaaaaaaagtttgatctcagttttatcatgaatgacttctccattgtgattcttgatttccttcatgggttaatttagaggttatgtatgtatgtctgtgtgtgtctgttgctaaggtcaccatggctgaccgggctgctgccgaaagggcctgcaaggatcccaatcccatcattgatggcagaaaggccaacgtgaacctggcatacttaggagcaaaaccaaggatcatgcaaccaggtgagaaatgtctgtctcccctacccccctctccaagaaccccccatttatgggtgggatgattctctaatttagaagacattgtctctttctgtagatgtgtttagtaaacttgaaccatggcttgttattctaccaaggcattaaacataaatgagagttgtaaatactcctctcaaggcttgaaatgtacctgtgtgcaaaaatggatcttgggggtcgtgcaggaagaagagttagtcatgtctcacctgtttcctatatggcatacatttgtagtctgctctcaattttccaatttttagccacctgtagataattcacatgtggattatataggccaaatttgtcgacttcagctaatatacttccctacttagtggtgtctctccttttagcaccagttgcactgtgctcagagcaggcagtctgacttgaacacagacccaagggaagcattaaggggtaggggcgatgtccaaatggtaaaggttttgtttttttttgagatggagtttcgttcttgttgcccaggctggagtgcaatgacgcgatctaggctcactgcaacctccgcctcctgggttcaagtgattgtcctgcctcagcctcccaagtagctgggattacaggaatgtgtcaccacacccagctaattttgtatttttagtagagacggggtttcactgtgttggtcaggctggtcttgaactcctgacctcaggtgatccacccgccttggcctcctaaagtgctgggattacaggcgtgagccaccgcgcctggcccaaaatggtaaaccttcaacataaaatcacagagtattagcaatcaaagaaaacagaaattgcacctactcaagaaccctatgctacagacaaagaaactgatgcttagagatgagaaatattttacttaagtcatgtagctattagttgccaaaagggagcagttttttgtctatgggaaaacaatacctcttggattatttacaacactgccctttgcattagcacaaatagttgagagttgatttttaagcaaatataatattttctttggtcaggcaactttacttgtaaagtttgattaaatcatatactcttcactcctaagcctttccatctgaaattttgggaagaggttgacacttccatccaaacgggtttaaaaggaaactaggttgtcaagaaaatgagtgtttgtgcaaatattcatgggcgttaaaaaaatttgtctaatacattgtgggtatgtagtatccagaaaaaaaacatttttttttgtgaatttttaatactggaaagtgtaaaacccataatcttccaaagcattgtctgaagcatcccagaacgttccaaattacaaagacttttcatagattctggttcaaaactgtgagaatatttgagatgtaacttcactgtcaaatcctgctgtacttaattttatttcatgaacattttcccaaggtctgattctgtgtctgtcaactaaaatttgcagatttttttaaagttttcaccttttctctgttaaaacctttattttgaatcaatgatagcatttataaaactgactttaaaaaatatacatacatatagtttctttaaagtggcgaaagaatgtttatcttttataagtgaaagttgtgattgcattttacatgtaggaataataaatgcctaaccatgcacaaataaagaatgtggtatattttgttgttaggttttgcctttggtgttcaacaacttcatccagcccttatacaaagacctttcgggtaagttgattaatcaggctttcttaagtttcagtatgactatcatttgttaacgtgcctctttgttatatacagataagatcatgatgtcacagaatggtcctagttatacaattttatatatatttgtgcatttttaatgctgatactataaatatttttagatgagtcaagttatctgaccaagtatgcagcctccaaacgagacaaattttgagcttatgattagcaaaacaaaacaaaaataccaggtatgacttgtgtacaagacgattgtgtcccatcaccattactactgttcgacttgactcaattttatttatctctttctaacctgacagggctcgtatacagttaagctaactgatcagaaggataccatgataagaggtggaatagttctgtttggaattaaactctgtaaccagtagactcagacacacaagctcagatggtgattaacgatgcatggatgttggtgttgttgtttttatttttcacaatagtcaatagcccttcgtgtgctttattttatttattacagtaatgaagtggtgttgatagctaaactgtgtgctttttacttaattgaaaagagctgtagtgaaagtgttttcttttcctcggggaaggggctctttgcgttttgattttggtggtggtgttgaggggtgttgacttttgactttgccatgggaagcaaagggcctttttttttttcttgcttttaagctgtagcctgtgacttccatttaaccacatttttgtagtgagtgctaaattgtgtgatttctctttgtcagaatacagattcattgcttgcatgtcctaagtttctgttttatgggaaattttctgggaatggggttctttcttttgcaatgtgtatccacttttaggccatctaatttaaaggttcaacaactctcatcagattttattaacacaagccagtttttttgagtagcgtcacactaactttcacctttcagatctaactcaggtgaacacctgttcatatttatgttgtctttgggtcctgtgattaccagttttcctgttccaaggtcctcatttctcttcctttcagaggttatatgggtgggagatcacccctcgcctcaatttatcctggacactagattctgttttggctctgttctacctgatcaacatgcctgtaactctaacgcaggatacttaaattttctttttaaatgttcatgttgttgaaagagtgttttatcctcaataataatttctcctctagcttaggacctaaaacatctacctgtgtaacctggaaggctggaatctgtctacatagatagatgtctaccatatgtgtctctaagaatagattttaaaaagaaattaataataacttgacttggaagagtaaagttatatttcccatgaccacctccttacaactctaacatatcggtacctcactgttattgccaactactactatttttttttttttttttttagctgtggaagagtcttctgttcaatatgatcagaaagggccttcctatatccatagctacttagcaaatccccatccaagccaacccaaatactgaaaacaagtggtgggttgatgggttcagctatttctgtgggtaattcatttagacttaactgtttaaggtgggattagggaggagagcatatatctgaaggtgtgatgtgagcttagtggtgggctcacactaataatcttgaaacagttataaaattgaatcaatcaacaggtagttgcatgtgtttattttttcactgtgccagtgggtgatgcaaaatgagggaattgcacattagtttgggaagcaaaagtcatgcaaaaaccagtcaagaattattaatttctaagtgacttggtattgtttcaagtacaaaaggaattcagagtagcaagaaaatagatgaagtggaggagttgaataaggctgtggtgtgcatggcaggtagactggcactagacttgcaccactgggtatgtcttaggcaggtaaaaggaagactggatatgtctggagggtataggatggagtgagccgaagcagaggcatagaggagaagactcagtgcgtggagaaaccagactgtctaaggtgggtcttgagagctgggcataggaatttggactgattgtggaagtccttatagatcagttcttaagaaagaaggtaacacaaggaaaattacaaccaccagtgtaagatgtgtcatggaaatttgaaagcaagtgtgttttgatagcaaccttttgtttttcacctcttctaatattaactgtctttataaaggatggtgattattagtgtgtgcataaattgtttgaacttgagattcttattagaaatagatagcattccaggtggaccaagtgagctttctctttcttttttaagaggttctcaacactgctctaatttcagagtctgtacctaacaaatcacacctggtcatcctaaattctgtttgaatgctacatgttcctattttaggcctggaaagaaacttgagaatttcaaatttgacttgacttacttgaaattctagtaactgaactgttcctttgaatgtggtccttaaatggcccccaggtgactttgggcctctgtagacaaaaccttggcctaaaaggaatttctgatgtagcacagtgggcagatgtagaaaaacttataaacttgctaaacaatattttatagtttatggttgaaagaagcatttccttattgaaaccttggaaaggggaggctacagatctgtgagtagaaatttctgggctagtggccaggtgcggtggctcatgcctgtaatcccagcactttgggaggccgaggtgggtggatcatgaggtcaggaggttgagagggcgagaccatcctggctaacacagtgaaaccccatctctaataaaaatacaaaaaattagctgggcgtggtggtgggcgcctgtagtccagctactcgggaggctgaggcaggagaatggcgtgaacccagaaggcggagcttgcagcgagccgagattgtgccactgcactccagcctgggcgacagagcgagactccatctcaaaaaaaaaaaccaaaaaacaaatttctgggctagtttagatgaggttgaacatacacttaaacattgactcagtacccctttaagtgctcagttttatgctgggtgtggtaacactgtgcttagtttttatagtgtccactaaggagggcaggagggactgtaactaacactaaccaaacatctgccatatggattgtaatgataagggatttacataaattacataatttaattctcacagtaagtgtttgttgaaggcattgttaatatcttttccaaaactaagacacagagaggttaattaacttccccctgatcacaggactgctaagtgttagaggctgagtctagattcagaatcatggtctgcctgacctcagaagctgcagtctttcctctttgtcaagctgtagccccaaaagacaaaactctattctctcataatccagtaagaaagccgataagcaccaagaagggctgtccatgtggcttcagataagagagacgaacatcaggacaatgagtgggattcagaagtcttcgtgaaggaagtagaagttgacctaggttttaaagaatggtggagggtagggaactcttttattgagtgcctactatatttggcattgtagactctctccacagataattttatttaatgcacagcaatccagggttgtgagattattagtccatttcacatatgcaaaataggaggctatgttaagacaacaaagcataaaaaaagaaacatatgagactaaacgataaagcaaatttctgaaacctctacacgtagtgagtgctagagctagggtgagtgggatttggatttcagcaggtagagacagtgtgaggattagcatctgggtgagggatgaatgacatgaataaagggcaagaatctgggaatgtgaagcttgtccaggaaacataaaggagcccagttgacctgggtttgaggaaaggtggaaggaaatactagaaaggtgttttggagccaggggcagtctggatgttttggcctagaatgccaaggcttaagaaatttgcactgagtttggttttttaggggccaaggattactttgagcaaatgaatgagatggagtatttgtcatcagttgctgtgagatagattggaaaaagaaaccgattaggatattgcttcaaaaccagttaggatgttgctttaaaagtcacatagagatgtcagtggaacctgaagagatgggaagaaaaggaaaatgtagtaaattttctttgaaaattaccaaaagtgtcttctttgcacttgtgctttttgtaactgctaggcatgcacgttttatggttaaggaaacgacattggtaaatgtctaataggagggagattcccagatcaccttaatgggtctcagactattaaaaagaactcctaattcaggatttcattccaaagtgccacttgaaccttcagtccaattctgccttccttcctgacccaggatattttaggctctccaaggtctttcgggttctccttttccctctaagccttaaaatgttctaggttttcatttgccttccagacgtctctttaacctttgccaaacttcatgtaggagtctgataatccagtgaactttctttatattctcattgtttgacattgtgaaatcaaaactatttcctaatgcaaagtaaatgcagagctaaagccccctaagcaaccttgcctgtcttttaatgcattcttcacaacactttctcctttttgtaggataaacattttcacttaacaccttaattcagagataccaacctagcacccacagtgttgttcttggaactttgagagtacaaggggttaaggtttcggtgtcagtgagatccgcactggctggcctttgcagctgatagatgttttactttggccttataaagacattttacccgctggaaacccttgtcgcatgacaggatcaaaaatagcccggctgcagcccctgctgtccatcaccacatgtaaactggcataagagtaggctctcttagccgtgtctacacgttgtagacacggcagcctccagggacagtgtttcataaagccacacctgttgttgcgagtgagccagtcacctgacctgcagcaaagttctgagtgtgtgcatgaccacgcgcctggtttctgacaagcagttaagaacatctctgagtgtcctcagaaacacttgatgacgtcgtttctcctggcactgggaaacatctacaagtctggtaaaattgagtgtatttttatcattggggccaaagtccactaccctctgagataaattcatcttttccatggatgttcgttaatgagtgctttcagtaaaactctgaaaaggcaaagcaaaatctatgtttctgaaaaatctacaagtgctgttttctgttttgagttctttgaaattgaaaggttgttacttgtccttgtacttcttacaagttggctttgccagcaaccacgtagtattatagtgtcatgtttttagatataaatatagggggctttaatggagattattttaaagaagaagataaatcagatatttatggtagtgtttcagatgggcagtaataaattgcaagagtgagaatggagatgcatggaatttttgtttaaatacctcttgtgatttatttttttaaccacattaacttttaagtaacgtcattggagtctgtcttgtattatctgtgaatgtataattttaaaagatacttggttttgtttgactttcttgaacttttgcaggataacacatgattatacaattgatatgatttgtacaaaatatcaacgaatagggcagatttcgaacataacactaacgtatttctgctaatttggaggatgaaacaagagttagtagatgtgcaaaaaaagtttccttgttatgtaaaccttttaaatttctattaattttagaaatatttagttgtacttccctgtcctgccttttccctcaacctcatcatttccccaaaaccccttctgtctgtcttgcatagagataaacaagctggctgaccctcaggtgcctgatagtcaaacaggtttggttgaccgttgtgtccttatgaattttatgggggaaaaaaaagagaaagaaagaaaataagtgtgctgcacatctctttgggaatcactgccatgaatgttaatagcatttttgtagcttcccacgacagcctcttgtgacagagtgagctgcactacagttcgtttgagaagtgtgattctgctgtgtttaccacacttgtgaacttttaaactcctttttcttattttgtcgcctgcttccttttttaattattatttttaataagggtgactgaggttgcagagagattaatgtgtgctgagtctgcatagcaatttcaagtagggttgccatgataataaacatacggattttgttaacgtttatgttaatttcgacaaactggtgatcaccccacagaagtgaataattatggactaccttttgagtcctatattcttggctaagaatttttctatttacttaattaaacattcttattttatgaactcattacaaaattttccaaagtcttcatcaattgagcacttgttaaacctctcttacgtgtgtagccctcatgaatcttgatttattttccaaactcttgataaactgtacttagcagctccaaataatcatacatgacaattctagcagggagggtgtcatcatatttttggggtccagttcttctgggggcactctatgactttggctactgcctgagtattctcaatggaccactagaaagtagatattgtggcaaccctatgctcataataaccactaaatttgagtttgttttatctttgaacaacatgctacagttaggaggtgactaccgcctgactttgttttccatttctcaacaggatacctgcccactatgtctatccgcaggcttttgtgcagccgggagtggtcattccacacgtccagccgacagcagctgccgcctccaccaccccttacattgattacactggagctgcatacgcacaatactcagcagctgctgctgctgccgccgccgctgctgcctatgaccagtacccctatgcagcctctccagctgctgcaggatatgttactgctgggggctatggctacgcagtccagcagccaatcaccgcagcggcacctgggacagctgccgccgccgctgcagcagctgctgccgctgcagcatttggccagtaccagcctcagcagctgcagacagaccgaatgcaatagaccagccatctgatcaaagttgaattgttttctctttccctcccaattttccaatttttagtagctaataagagagttaacattgacttaacagctttaaaaaaaaaaacagccatgctattgtgaagcagagttattattttttttatactccaggtagtgttctagatgagaaagaggtaagaatgaggggaatgggcacaattttggaaatcaatcccaaagagcctgagtaaatgaaaggccactacgaaatgacgccaggagtaacaacggaacttcacttttgtaacgggatttttatttttgctctttttatagtatcagggaagcaaactgccttttacaagttagaaaatgctgtttgaatctagttgaaccagggaatacagagcgagcaatatgtagcttgaattacatttaaaagcagattttttacaaacaaaatggcgaaagcactgattgtcatttttagcagtcacttaaggcctatagaacttttttcaagtcggaaggtcctgttcttactatctcaaaaatgggcatcgaacaatcaatctaggagcgtggcagtgggtaaaatggtggacaggcaccaaagctattttctcatctgtcctgtggatgagtggaactgtggaacaagtgatgtggaattaatgggtgcaacagctgtacagacaatcaataacacacacagttctggaaagaacacatcacttgtgcttgtttgatgagcttgtcacattctaatccctctccccatcctgtttcaattttgggaaacttgtatctgctggtgtcagcaattctgattctgaaataggatcaggcttttactacaacagccttctctttctatttattgtgtcgactcgtggcttatgaataaaggcaggcaaagtttgcagaactgtcttaaggacatttattttttttccctttttaagtaattttacactttagtatctatttcagagtcatatttaaaactatttattagtgactacagtacatgagacaacaaggttactgagattacaattcttcaaaggttaatgataatgtggtttatactgtgccttaatagtaatgctatttaagatatttatttttaagttttactatgctgcactctaaagaaaggaactttagatgtgacactgtaaaattatgtattcatctcatggcataaattatttagtaagtctagatgtagcgtattaaatattaacctattcaactaaagatgttgacttggatttatttaaattcatatgtgcactgtataagagagtactcttacattaacacattttagtttagtttttagatttttttttaaacgatatattgctagtttcatgcttcctcctctgattttgcctgtgtagatttcatttattggtaattcattagtttaacactattatgtagtttttaaatgctgctttacatttttcttctgaaactgcaatacttgcaatttttattcttaaactaaattgaatactattttacactgtattggatttttatacttgaacaatttcatacaagggaagacaggttagcatttttatggactttctccattatcactggatttactttaagtattcccatactagacagtgttatgtaatgtagacatgactctcctgtgcaaattatttattcgttgtgtatattgctttataacatttcagatcttctaatctattcacttgtattaaatataatttttaaaaacttctgttgcattggctgtttctaattctgtagtaaaggtcaatgatttctcttttgaacttgaatcaatagccatgattgatacgtaacattttcagtgagcataatactctcttggatgtcctggttttgtgcctttaaagtaagtttcactaactgaatatctaccttatacttagcaaatgatataggtcatcccctgtcccaccaggggagccctaaaggacttcgtatcactgaaaactggttagctgaaagatggcttggaaggcgtgggccagtagtagagaggactagtcagaagtgggaccagcccagtggtgctcgggtcagctaaactcaggtttgagctggactccccacttttcagcaatgttagtgaagccaattgctcttcgtaattcacctttttttttttttttttttttgagacagagtcttgctctgtcacccagtctggagtgcagtggcacgacctcggctcactgcaagctccgcctcccaggttcacgccattctcctgccttagcctcccgcgtagctgggactgcaggcacccgccacctcgcctggctaattttttgtatttttagtagagac

SEQ ID NO: 13 - DNA, 22 bp, synthetic construct
  source 1..22: mol_type = other DNA
atcgctgctgagtgaaccacag

SEQ ID NO: 14 - DNA, 22 bp, synthetic construct
  source 1..22: mol_type = other DNA
ctactctctgcctcacaggtca

SEQ ID NO: 15 - DNA, 22 bp, synthetic construct
  source 1..22: mol_type = other DNA
aacctcgatggcttggtggcaa

SEQ ID NO: 16 - DNA, 22 bp, synthetic construct
  source 1..22: mol_type = other DNA
tcaggatgtggcactcatagcc

SEQ ID NO: 17 - DNA, 20 bp, synthetic construct
  source 1..20: mol_type = other DNA
aagcatggcctgtacaacct

SEQ ID NO: 18 - DNA, 19 bp, synthetic construct
  source 1..19: mol_type = other DNA
gggttcacacaccagcact

SEQ ID NO: 19 - DNA, 19 bp, synthetic construct
  source 1..19: mol_type = other DNA
ccaaggatcatgcaaccag

SEQ ID NO: 20 - DNA, 20 bp, synthetic construct
  source 1..20: mol_type = other DNA
gcaggtatcccgaaaggtct

SEQ ID NO: 21 - DNA, 23 bp, synthetic construct
  source 1..23: mol_type = other DNA
cttcagttcgtgtgtggagacag

SEQ ID NO: 22 - DNA, 16 bp, synthetic construct
  source 1..16: mol_type = other DNA
cgccctccgactgctg

SEQ ID NO: 23 - DNA, 18 bp, synthetic construct
  source 1..18: mol_type = other DNA
gaataacacagccgatgg

SEQ ID NO: 24 - DNA, 18 bp, synthetic construct
  source 1..18: mol_type = other DNA
ccaggtcactatcaggag

SEQ ID NO: 25 - DNA, 18 bp, synthetic construct
  source 1..18: mol_type = other DNA
caccaactgggacgacat

SEQ ID NO: 26 - DNA, 18 bp, synthetic construct
  source 1..18: mol_type = other DNA
acagcctggatagcaacg

SEQ ID NO: 27 - DNA, 19 bp, synthetic construct
  source 1..19: mol_type = other DNA
tatggcgacccgcagccct

SEQ ID NO: 28 - DNA, 21 bp, synthetic construct
  source 1..21: mol_type = other DNA
catctcgagcaagacgttcag

(19) United States
(12) Patent Application Publication
(10) Pub. No.: US 2023/0332237 A1
(43) Pub. Date: Oct. 19, 2023

(54) Molecular Biomarkers for Predicting Patient Response to Steroid Therapy

(71) Applicant: Florida State University Research Foundation, Inc., Tallahassee, FL (US)

(72) Inventors: Akash GUNJAN, Tallahassee, FL (US); Yuna SON, Waltham, MA (US)

(21) Appl. No.: 18/300,477

(22) Filed: Apr. 14, 2023

Related U.S. Application Data
(60) Provisional application No. 63/330,965, filed on Apr. 14, 2022.

(51) Int. Cl. C12Q 1/6883

(52) U.S. Cl. CPC C12Q 1/6883; C12Q 2600/106; C12Q 2600/158

(57) ABSTRACT

The invention concerns molecular biomarkers useful for screening individuals for steroid resistance and steroid sensitivity in the selection of treatments for keloids and other conditions. Aspects of the invention include a method for determining whether or not an individual will respond beneficially to a steroid therapy; a method for selecting a therapy for an individual having a condition, using the screening method; a method for treating a condition of an individual, using the screening method; and a detection device useful for detecting expression levels of the biomarkers.

### CROSS-REFERENCE TO RELATED APPLICATIONS

This application claims the benefit of U.S. Provisional Application No. 63/330,965, filed on Apr. 14, 2022, which is incorporated herein by reference in its entirety.

### GOVERNMENT SUPPORT

This invention was made with government support under Grant No. NIH UL1TR001427 (Subaward SUB00002344) awarded by the Clinical and Translational Science Award (CTSA) Precision Health Initiative. The government has certain rights in the invention.

### CROSS-REFERENCE TO SEQUENCE LISTING

This application contains a sequence listing filed in ST.26 format entitled “930603-1250_Sequence_Listing.xml” created on Mar. 24, 2023 and having size 504,513 bytes. The content of the sequence listing is incorporated herein in its entirety.

### BACKGROUND

Steroids are used to treat a wide variety of inflammatory, hyperproliferative, autoimmune and pruritic disorders. Topical steroids are used for treating multiple skin disorders, while steroid injections are used for keloid therapy. However, there is a wide variation in patient response to steroid therapy (topical and otherwise) that is likely due to genetic differences between patients, although the genes involved have not been previously identified. Given that no tests for steroid responsiveness are available and steroid therapy often lasts for years, there is a dire need for a rapid molecular test to determine the patient's response to steroids prior to initiating therapy.

Keloids are disfiguring, painful and itchy but benign fibrotic skin lesions characterized by excessive dermal fibroblast proliferation and collagen deposition. They occur in susceptible individuals due to abnormal wound healing. Keloids are understudied, difficult to treat and predominantly affect dark-skinned individuals. Keloids are relatively common in certain racial and ethnic populations such as African, Asian, and Hispanic descent with an estimated incidence of ˜ 1/30 for African Americans and ˜ 1/625 in the overall US population (1, 19). Moreover, it has been reported that people of African ancestry show higher frequency or more severe and exaggerated response to injury in keloid formation (20). However, the molecular mechanisms involved in driving keloid formation in susceptible individuals are unclear. Currently there are no standardized treatments available for keloids and they have very high recurrence rates upon surgical resection alone. Intralesional steroids are commonly used, albeit with highly variable responses.

Wound healing leads to tissue repair or regeneration after skin injury. It is a complicated and dynamic process with three main time dependent phases: inflammation, proliferation, and remodeling. When the skin is damaged, many cytokine mediators are activated, and inflammatory cells, epithelial cells, and fibroblasts are recruited during the inflammatory phase (1). The proliferation and relocation of fibroblasts into the wound matrix starts around day 4 or 5 after wound formation, during the second stage of the wound healing (2, 3). The remodeling phase usually begins three weeks after tissue injury, and it is responsible for intra- and interpersonal differences in scar formation. Abnormal scarring such as keloids and hypertrophic scars can develop as a result of the imbalance between production and degradation of collagen during the remodeling phase (3).

Keloids are benign dermal fibro-proliferative lesions that extend beyond the site of injury and invade the adjacent normal dermis; in contrast, hypertrophic scars do not extend beyond the original wound edges (4). Although a keloid is classified as a benign dermal growth, its behaviors share the biological features of malignant cells in terms of local invasion and hyper-proliferation (2). Previous studies have shown that the proinflammatory genes are upregulated via an inflammatory response in keloid formation (5). In the normal wound healing process, the balance between inflammation and extracellular matrix formation is well maintained, while the control of fibroblast activity is lost in keloids (5, 6).

The management of keloids is suboptimal after surgical treatment alone with a recurrence rate of up to 100% (7). Although intralesional corticosteroids are widely used as a post-surgical adjunctive therapy (8-11), there are no systemic studies on the effects of steroid treatments for keloid therapy. Furthermore, previous data from clinical studies (12, 13) and in vitro results (14) suggested that different individuals may show highly variable effects of steroid treatments (15, 16). Although previous research has shed some light on the possible molecular mechanisms involved in keloid pathology, the understanding of the pathophysiology of keloids and the variable effects of steroid therapy remains incomplete (17, 18).

### BRIEF SUMMARY OF THE INVENTION

To address the lack of a molecular test to determine a patient's likely response to steroids prior to initiating therapy, the inventors have used an unbiased transcriptomic approach using RNA sequencing (RNA-seq) performed on total RNA isolated from human patient cells to identify genes that are differentially expressed between patients whose cells proliferate slowly upon steroid treatment (i.e., they are steroid responsive), versus those that do not. The inventors applied a whole genome transcriptomic approach on total RNA isolated from the same set of patient derived keloid cells to provide new insights into the molecular pathways that contribute to keloid pathogenesis and determine response to steroid therapy. Additionally, the inventors'study identifies biomarkers for response to steroid therapy that can be used to predict patient response to steroid therapy prior to initiating treatment.

Using quantitative reverse transcription-polymerase chain reaction (qRT-PCR), the inventors have determined that each of the four genes Melanoma Cell Adhesion Molecule (MCAM), WAP Four-Disulfide Core Domain 1 (WFDC1), Insulin Like Growth Factor Binding Protein 2 (IGFBP2), and RNA Binding Motif Protein 24 (RBM24) are poorly expressed in steroid responsive patient cells, and each of insulin-like growth factor 1 (IGF1) and Transcriptional Coactivator and Phosphatase (EYA4) are highly expressed, while the opposite is seen in steroid resistant patient cells (as shown in FIGS. 2A and 2B). This suggests that the expression patterns of these genes can serve as excellent biomarkers for predicting response to steroid therapy in patients and can be used in a molecular test, such as a qRT-PCR based rapid molecular test, to determine patients' response to steroids in just a few hours. Following this, only the patients categorized as steroid responsive are selected to undergo steroid therapy, while other patients categorized as steroid resistant can avoid the potentially harmful steroids and obtain alternative (non-steroidal) therapies. Incorporation of this predictive tool into clinical decision making will reduce possible adverse effects of steroid treatment, while increasing patients' compliance significantly.

One aspect of the invention concerns a method for determining whether or not an individual will respond to a steroid therapy, the method comprising determining the expression level of one or more genes selected from among MCAM, WFDC1, IGF1, IGFBP2, EYA4, and RBM24 in a sample obtained from the individual, wherein a higher level of expression of one or both of IGF1 and EYA4, or a lower level of expression of one or more of MCAM, WFDC1, IGFBP2, and RBM24, relative to a reference expression level that is representative of the level of expression of the one or more genes in an individual who is steroid resistant, indicates that the individual is steroid sensitive (and thus predicted to be clinically responsive to steroid therapy), and wherein a lower level of expression of one or both of IGF1 and EYA4, or a higher level of expression of one or more of MCAM, WFDC1, IGFBP2, and RBM24, relative to a reference expression level that is representative of the level of expression of the one or more genes in an individual who is steroid sensitive, indicates that the individual is steroid resistant (and thus predicted to be clinically non-responsive to steroid therapy).

Another aspect of the invention concerns a method for selecting a therapy for an individual having a condition, comprising: receiving results of the aforementioned determination method; and selecting a steroid therapy for the individual if the individual is steroid sensitive (predicted to be clinically responsive to steroid therapy), or withholding steroid therapy from the individual and optionally selecting a non-steroid therapy for the individual if the individual is steroid resistant (predicted to be clinically non-responsive to steroid therapy).

Another aspect of the invention concerns a method for treating a condition in an individual who is classified as having a steroid sensitive phenotype (steroid responsive phenotype) or a steroid resistant phenotype, the method comprising:
(a) identifying the steroid sensitive phenotype or steroid resistant phenotype for the individual, wherein said identifying relies on a determination of the expression level of one or more genes selected from among MCAM, WFDC1, IGF1, IGFBP2, EYA4, and RBM24 in a sample obtained from the individual,
   wherein a higher level of expression of one or both of IGF1 and EYA4, or a lower level of expression of one or more of MCAM, WFDC1, IGFBP2, and RBM24, relative to a reference expression level that is representative of the level of expression of the one or more genes in an individual who has a steroid resistant phenotype, indicates that the individual has a steroid sensitive phenotype, and
   wherein a lower level of expression of one or both of IGF1 and EYA4, or a higher level of expression of one or more of MCAM, WFDC1, IGFBP2, and RBM24, relative to a reference expression level that is representative of the level of expression of the one or more genes in an individual who has a steroid sensitive phenotype, indicates that the individual has a steroid resistant phenotype; and
(b) treating the individual classified as having a steroid sensitive phenotype with a steroid that is suitable for the condition (e.g., administering a steroid by any suitable route), or withholding steroid treatment from the individual classified as having steroid resistant phenotype, and optionally treating the individual classified as having a steroid resistant phenotype with a non-steroidal treatment (e.g., administering a non-steroid agent by suitable route) that is suitable for treatment of the condition.

Another aspect of the invention concerns a method for determining the expression level of one, two, three, four, five, or all six genes selected from among MCAM, WFDC1, IGF1, IGFBP2, EYA4, and RBM24 in a sample obtained from an individual, wherein the cell sample comprises one or more cell types selected from among fibroblasts, epithelial cells, keratinocytes, and melanocytes.

Another aspect of the invention concerns a detection device comprising one or more capture probes associated with a substrate (e.g., a solid surface such as a bead, chip, plate, or other substrate), wherein the one or more capture probes hybridize with target nucleic acids encoding one or more of MCAM, WFDC1, IGF1, IGFBP2, EYA4, and RBM24, or wherein the one or more capture probes have an affinity for target polypeptides of one or more of MCAM, WFDC1, IGF1, IGFBP2, EYA4, and RBM24, and wherein the detection device includes capture probes that hybridize with no more than 500 total nucleic acid targets or that have affinity for no more than 500 total target polypeptides.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIGS. 1A-1C. RNA-seq reveals differential gene expression profiles of steroid sensitive and resistant keloids following steroid treatment. FIG. 1A is an MA plot for the differential expression of genes in steroid sensitive keloids following steroid treatment. FIG. 1B is an MA plot for the differential expression of genes in steroid resistant keloids following steroid treatment. FIG. 1C is a heatmap of top 20 differentially expressed genes in steroid resistant keloids compared to steroid sensitive keloids. Up/Down: The number of genes upregulated/downregulated in keloids. Red color denotes higher mean expression levels and blue color denotes lower mean expression levels (log2FC: log2 fold change).

FIGS. 2A and 2B. Differential expression of specific genes in steroid sensitive (SEN) versus resistant (RES) keloid fibroblasts. FIG. 2A shows that the mRNA levels of each of MCAM, WFDC1, IGFBP2, and RBM24 are upregulated in RES keloids. Primary keloid fibroblasts were obtained and processed for qRT-PCR as described in Methods (KPA, P5, P17 (SEN keloids); P16, P21, P24 (RES keloids). UT: untreated; TA: steroid treated. Relative expression was normalized relative to ACTB gene expression. Error bars represent standard deviation of the mean (mean ±SD) obtained from 3 experiments. Significant differences are indicated by the asterisk (*P<0.05; **P<0.005). FIG. 2B shows that the mRNA levels of IGF1 and EYA4 are downregulated in RES keloids. Data was obtained as described for FIG. 1A.

FIGS. 3A-3C show that IGF1, IGFBP2, MCAM, and EYA4 are present in human saliva. FIG. 3A shows the presence of MCAM and IGF1 in saliva samples from 3 different individuals using Western blot analysis. Tubulin has been previously reported to be present in saliva and its levels serve as a loading control. The use of twice as much saliva sample results in the detection of roughly twice as much IGF1 signal. FIG. 3B shows measurement of IGFBP2 levels in saliva and cell culture supernatants using highly specific sandwich ELISA kits. Colorimatically detected IGFBP2 levels are provided in pg/mL of saliva or cell culture supernatant. FIG. 3C shows measurement of EYA4 levels in saliva in ng/mL using ELISA. Twice the amount of saliva results in roughly twice the signal.

### BRIEF DESCRIPTION OF THE SEQUENCES

SEQ ID NO:1 is the amino acid sequence of human Melanoma Cell Adhesion Molecule (MCAM), a.k.a. cell surface glycoprotein MUC18, UniProt Accession Number P43121.
SEQ ID NO:2 is the amino acid sequence of human WAP Four-Disulfide Core Domain 1 (WFDC1), a.k.a., PS20, UniProt Accession Number Q9HC57.
SEQ ID NO:3 is the amino acid sequence of human insulin-like growth factor one (IGF1), a.k.a. somatomedin C, UniProt Accession Number P05019.
SEQ ID NO:4 is the amino acid sequence of human insulin-like growth factor binding protein 2 (IGFBP2), a.k.a. aka IBP-2 and BP2: UniProt Accession Number P18065.
SEQ ID NO:5 is the amino acid sequence of human transcriptional coactivator and phosphatase 4 (EYA4), a.k.a. eyes absent homolog 4, UniProt Accession Number 095677.
SEQ ID NO:6 is the amino acid sequence of human RNA-binding motif protein 24 (RBM24), a.k.a. RNPC6, UniProt Accession Number Q9BX46.
SEQ ID NO:7 is the nucleic acid sequence of human MCAM.
SEQ ID NO:8 is the nucleic acid sequence of human WFDC1.
SEQ ID NO:9 is the nucleic acid sequence of human IGF1.
SEQ ID NO:10 is the nucleic acid sequence of human IGFBP2.
SEQ ID NO:11 is the nucleic acid sequence of human EYA4.
SEQ ID NO:12 is the nucleic acid sequence of human RBM24.
SEQ ID NOs:13-28 are forward and reverse primers used in this study (Table 3).

### DETAILED DESCRIPTION OF THE INVENTION

In the study described in the Examples, the inventors used RNA-seq in an unbiased effort to identify the genes and molecular pathways underlying keloid pathogenesis and their differential response to steroid therapy. Although previous studies suggest that aberrant wound healing processes may play a key role in keloid pathogenesis, the details of molecular pathways involved in keloid pathogenesis are largely unknown. Furthermore, while intralesional steroid injections have widely been used for the therapy of excessive scars since the mid-1960s (22), the understanding of the pathophysiology of keloids and the variable effect of steroid therapy is still very minimal (17, 18).

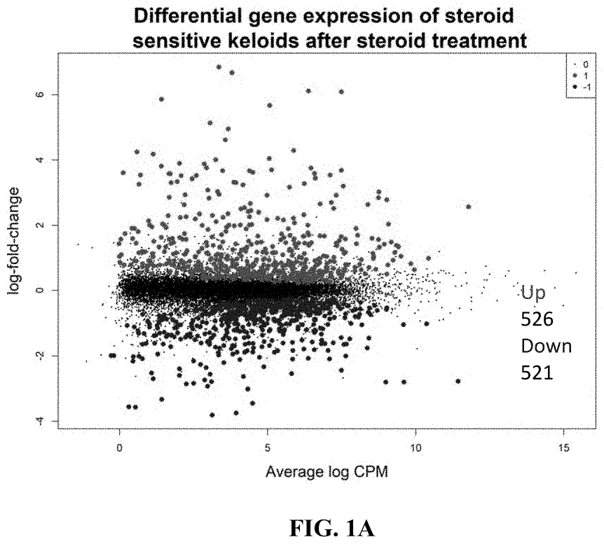

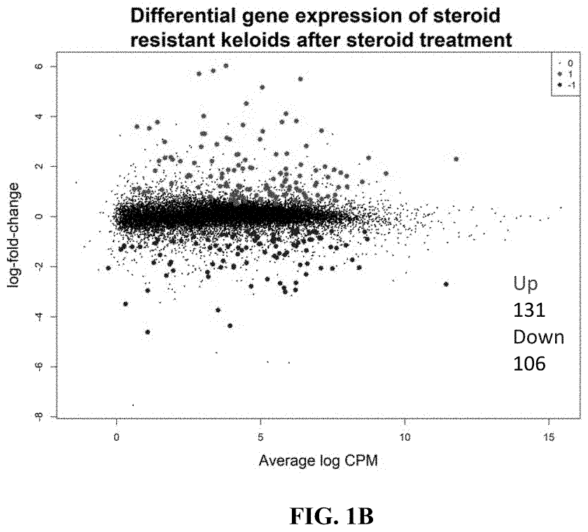

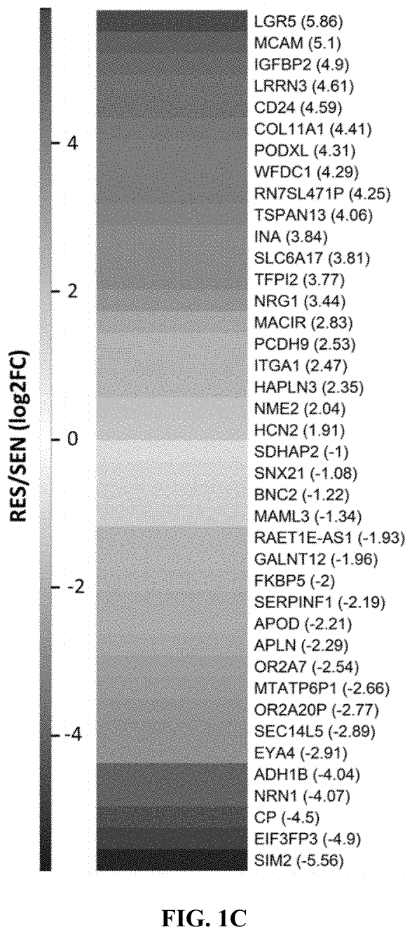

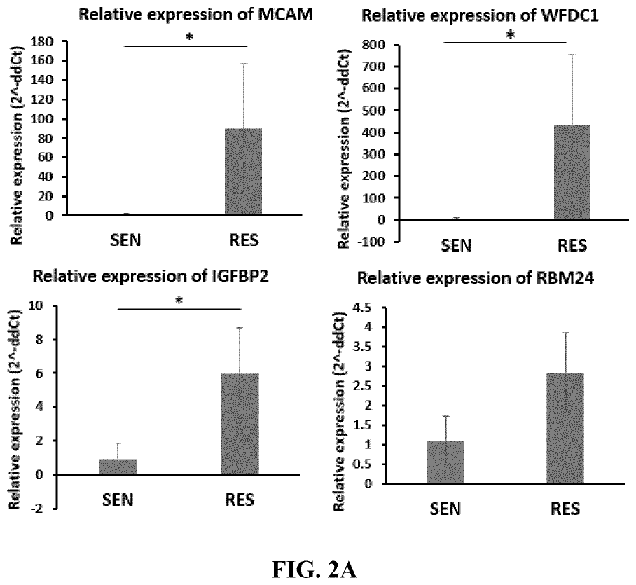

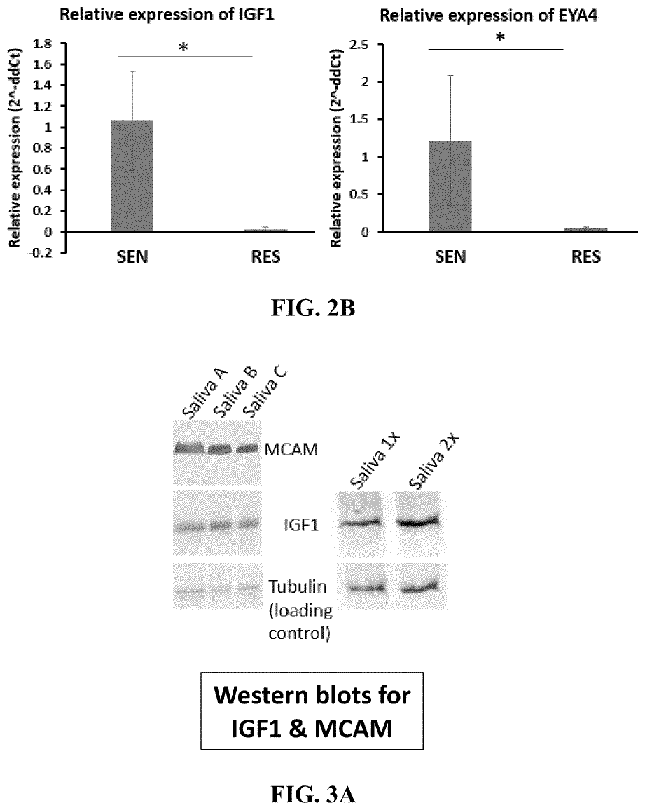

There has been little or no significant basic research to date aimed at dissecting the variable effects of steroids in keloid therapy. Previously, numerous studies have reported that different individuals show highly variable responses to steroid treatment for keloids (12-15, 25). In the study described in the Examples, the inventors have identified 526 upregulated and 521 downregulated genes in steroid sensitive keloid fibroblasts following Triamcinolone Acetonide (TA) treatment, while there were 131 upregulated and 106 downregulated genes in steroid resistant keloid fibroblasts after TA treatment (FIGS. 1A-C). Subsequent qRT-PCR confirmed the RNA-seq results for several of these genes, with EYA4 and IGF1 being downregulated, while MCAM, WFDC1, IGFBP2, and RBM24 were upregulated in steroid resistant keloid fibroblasts (FIGS. 2A and 2B).

Elevated MCAM was first discovered in metastatic melanoma, but a recent study found that MCAM is highly upregulated while estrogen receptor (ER) α is decreased in response to tamoxifen (a selective estrogen receptor modulator, SERM) resistant breast cancer (26). Here, the inventors have shown that MCAM is upregulated in steroid resistant keloid fibroblasts, and it suggests that MCAM may serve as a potential biomarker for steroid sensitivity for keloid therapy and possibly other diseases treated with steroids. Furthermore, based on our RNA-seq and qRT-PCR analyses, the inventors discovered that IGF1 expression is downregulated, while IGFBP2 is upregulated in steroid resistant keloids. Surprisingly, it has been reported that treatment with the corticosteroid prednisolone increases serum IGF1 concentration (24) and reduces IGFBP1 and IGFBP2 (23, 24). This suggests that the steroid resistant keloids are indeed showing different downstream molecular pathways following steroid treatment compared to the steroid sensitive cells. While this is a preliminary study with a small sample size, following further studies, the inventors are confident that the expression levels of several of these genes can be used as molecular biomarkers for detecting steroid insensitivity in patients.

The RNA-seq analysis of keloid cells expands the knowledge of the molecular mechanisms involved in keloid pathogenesis and in the response of keloids to steroid treatment. The inventors have uncovered multiple genes involved in several molecular pathways that appear to contribute strongly to keloid pathogenesis. More importantly, the inventors have identified several molecular biomarkers for steroid resistance in individuals. This provides the opportunity to determine steroid sensitivity of individual patients before initiating treatment with steroids. Having this available will reduce possible adverse effects of steroid treatment, while improving patients' compliance.

The various aspects of the invention are based on the inventors' determination that the levels of expression (at the transcript (mRNA) or protein level) of the following six genes can be used as predictive biomarkers for steroid responsiveness: (1) MCAM—melanoma cell adhesion molecule, also known as CD146 (cluster of differentiation 146) or cell surface glycoprotein MUC18; (2) WFDC1—WAP four-disulfide core domain 1, also known as PS20; (3) IGF1—insulin-like growth factor one, a.k.a. somatomedin C; (4) RBM24—RNA-binding motif protein 24, also known as RNPC6; (5) IGFBP2—insulin-like growth factor binding protein 2, also known as IBP-2 and BP2; and (6) EYA4—EYA transcriptional coactivator and phosphatase 4, also known as eyes absent homolog.

One aspect of the invention concerns a method for determining whether or not an individual will respond (positively) to a steroid therapy, the method comprising determining the expression level of one or more genes selected from among MCAM, WFDC1, IGF1, IGFBP2, EYA4, and RBM24 in a sample obtained from the individual,

wherein a higher level of expression of one or both of IGF1 and EYA4, or a lower level of expression of one or more of MCAM, WFDC1, IGFBP2, and RBM24, relative to a reference expression level that is representative of the level of expression of the one or more genes in an individual who is steroid resistant, indicates that the individual is steroid sensitive (and thus predicted to be clinically responsive to steroid therapy), and

wherein a lower level of expression of one or both of IGF1 and EYA4, or a higher level of expression of one or more of MCAM, WFDC1, IGFBP2, and RBM24, relative to a reference expression level that is representative of the level of expression of the one or more genes in an individual who is steroid sensitive, indicates that the individual is steroid resistant (and thus predicted to be clinically non-responsive to steroid therapy).

The cells of the sample can be from a diseased individual or a normal individual (e.g., prior to development of a disease or condition), and can be normal cells or abnormal or diseased cells, e.g., from normal tissue or abnormal or diseased tissue. Without being limited by theory, it is proposed that the response to steroids is genetically/epigenetically encoded and therefore will be reflected in cells obtained from either normal or diseased tissues.

The cells may be relevant to a condition that the subject has. In some embodiments, the cells are cells of a keloid, such as fibroblasts. Keloid fibroblasts may be from any site in keloid tissue (peri-lesional, intra-lesional and/or extra-lesional sites).

As will be understood by one of skill in the art, there are over 200 cell types in the human body. It is believed that the methods of the subject invention can be used to screen samples of any of these cell types. For example, any cell arising from the ectoderm, mesoderm, or endoderm germ cell layers can be proliferated using methods of the subject invention.

In some embodiments, the sample of cells includes buccal cells, or cells of a surgical biopsy. In some embodiments, the cell sample includes one or more of fibroblasts, epithelial cells, keratinocytes, and melanocytes. Fibroblasts and epithelial cells tend to be easy cells to grow from a tissue where they are found, and as such are very convenient to use in the screening method of the invention.

Advantageously, cells may be sampled and screened using the screening method of the invention at any time. For example, a cell sample from a subject that has a condition that is potentially treatable with a steroid may be screened; however, it may also be desirable to screen cell samples from subjects in advance when they are still healthy for their response to steroids, and use this information when the need arises to treat them for a condition that is at least potentially treatable using steroids (i.e., in which treatment with a steroid is an option).

Optionally, the screening method further includes, prior to determining the expression level of the one or more genes in the sample, obtaining the sample directly from the individual.

The sampling step may include preparing the sample for the determining step, e.g., by dissociating cells of the sample of cells (e.g., enzymatically, using an enzyme such as trypsin, collagenase, or hyaluronidase; or mechanically, by cutting, pipetting).

Another aspect of the invention concerns a method for selecting a therapy for an individual having a condition, comprising: receiving results of the screening method described above; and selecting a steroid therapy for the individual if the individual is steroid sensitive, or withholding steroid therapy from the individual and optionally selecting a non-steroid therapy for the individual if the individual is steroid resistant. The individual may have already undergone therapy (a steroidal therapy or non-steroidal therapy) for the condition, and the selection method is utilized to determine the next therapy, or determine whether a current therapy should be changed or maintained; or the individual may have not yet received therapy for the condition.

Another aspect of the invention concerns a method for treating a condition in an individual who is classified as having a steroid sensitive phenotype (steroid responsive phenotype) or a steroid resistant phenotype, the method comprising:
(a) identifying the steroid sensitive phenotype or steroid resistant phenotype for the individual, wherein said identifying relies on a determination of the expression level of one or more genes selected from among MCAM, WFDC1, IGF1, IGFBP2, EYA4, and RBM24 in a sample obtained from the individual,
   wherein a higher level of expression of one or both of IGF1 and EYA4, or a lower level of expression of one or more of MCAM, WFDC1, IGFBP2, and RBM24, relative to a reference expression level that is representative of the level of expression of the one or more genes in an individual who has a steroid resistant phenotype, indicates that the individual has a steroid sensitive phenotype, and
   wherein a lower level of expression of one or both of IGF1 and EYA4, or a higher level of expression of one or more of MCAM, WFDC1, IGFBP2, and RBM24, relative to a reference expression level that is representative of the level of expression of the one or more genes in an individual who has a steroid sensitive phenotype, indicates that the individual has a steroid resistant phenotype; and
(b) treating the individual classified as having a steroid sensitive phenotype with a steroid that is suitable for the condition (e.g., by administering a steroid to the individual by any suitable route), or withholding steroid treatment from the individual classified as having steroid resistant phenotype, and optionally treating the individual classified as having a steroid resistant phenotype with a non-steroidal treatment (e.g., by administering a non-steroid drug or other agent to the individual by any suitable route) that is suitable for treatment of the condition.

Another aspect of the invention concerns a method for determining the expression level of one, two, three, four, five, or all six genes selected from among MCAM, WFDC1, IGF1, IGFBP2, EYA4, and RBM24 in a sample obtained from an individual, wherein the cell sample comprises one or more cell types selected from among fibroblasts, epithelial cells, keratinocytes, and melanocytes.

Another aspect of the invention concerns a detection device, such as a microarray or immunoassay device, comprising one or more capture probes associated with a substrate (e.g., (e.g., a solid surface such as a bead, chip, plate, or other substrate), wherein the one or more capture probes hybridize with target nucleic acids encoding one or more of MCAM, WFDC1, IGF1, IGFBP2, EYA4, and RBM24, or wherein the one or more capture probes have an affinity for target polypeptides of one or more of MCAM, WFDC1, IGF1, IGFBP2, EYA4, and RBM24, and wherein the detection device includes capture probes that hybridize with no more than 500 total nucleic acid targets or that have affinity for no more than 500 total target polypeptides. The detection device may be used, for example, to determine the expression level of one or more of the genes in accordance with any of the methods of the invention described herein.

In the methods of the invention (e.g., method for determining whether or not an individual will respond to a steroid therapy; method for selecting a therapy for an individual having a condition; and method for treating a condition of an individual), the step of determining the expression level of one, two, three, four, five, or all six genes selected from among MCAM, WFDC1, IGF1, IGFBP2, EYA4, and RBM24 may be carried out by the party potentially administering the steroid therapy to the individual, or the step of determining the expression level of one, two, three, four, five, or all six genes selected from among MCAM, WFDC1, IGF1, IGFBP2, EYA4, and RBM24 may be conducted by a third party, such as a diagnostic laboratory. Results of the expression level determination may be conveyed in any form or format that conveys the results of the test, such as digital or electronic, paper, verbally (in-person or telephone), etc.

Steroids are widely used for a variety of medical conditions, in various dosages and delivery routes (Ericson-Neilsen W et al., The Oschner Journal, 2014, 14:203-207; Liu D et al., Allergy, Asthma & Clinical Immunology, 2013, 9(30); Shaikh S et al., ISRN Anesthesiology, 2012, Article ID 985495, which are incorporated herein by reference in their entireties). Any individual with a condition, or who may develop a condition in the future, in which steroids are a potential treatment option can benefit from the methods of the invention. Conditions that may be treated with one or more steroids include, but are not limited to, immune disorders, inflammatory disorders, hyper-proliferative disorders, dermatological disorders, and pain. Examples of immune disorders or inflammatory disorders include rheumatoid arthritis, lupus erythematosus. Examples of dermatological disorders that may be treated using the method of the invention include keloids, psoriasis, eczema, and dermatitis. In some embodiments, the dermatological disorder is a hyper-proliferative skin condition such as one or more keloids, hypertrophic scars, or tumors.

Steroids are used to treat a variety of inflammatory and non-inflammatory conditions. Corticosteroid drugs are chemically modified versions of natural glucocorticoids. Examples include cortisone, flurohydrocortisone, hydrocortisone, prednisolone, prednisone, methylprednisolone, dexamethasone, and betamethasone.

Two main classes of corticosteroids include glucocorticoids and mineralocorticoids. Glucocorticoids affect carbohydrate, fat, and protein metabolism, and have anti-inflammatory, immunosuppressive, anti-proliferative, and vasoconstrictive effects. Mineralocorticoids are primarily involved in the regulation of electrolyte and water balance by modulating ion transport.

Corticosteroids are generally grouped into four classes based on their chemical structure. Allergic reactions to one member of a class typically indicate an intolerance to all members of the class (the Coopman classification). Group A corticosteroids (hydrocortisone type) include, for example, hydrocortisone, hydrocortisone acetate, cortisone acetate, tixocortol pivalate, prednisolone, methylprednisolone, and prednisone. Group B corticosteroids (acetonides and related substances) include, for example, amcinonide, budesonide, desonide, fluocinolone acetonide, fluocinonide, halcinonide, and triamcinolone acetonide. Group C corticosteroids (betamethasone type) include, for example, beclomethasone, betamethasone, dexamethasone, fluocortolone, halometasone, and mometasone. Group D corticosteroids include the Group D₁ corticosteroids (halogenated) such as alclometasone diproprionate, betamethasone dipropionate, betamethasone valerate, clobetasol propionate, clobetasone butyrate, fluprednidene acetate, and mometasone furoate; and Group D₂ (labile prodrug esters), such as ciclesonide, cortisone acetate, hydrocortisone aceponate, hydrocortisone acetate, hydrocortisone buteprate, hydrocortisone butyrate, hydrocortisone valerate, prednicarabate, and tixocortal pivalate.

As used herein, the term “glucocorticoid” refers to a class of steroid hormones that bind to a glucocorticoid receptor, which is present in almost all vertebrate animal cells. The glucocorticoid receptor is also known as a nuclear receptor subfamily 3, group C, member 1 (NR3C1), which is a receptor to which cortisol and other glucocorticoids bind. The glucocorticoid receptor may have an amino acid sequence of NP_000167 (human) and NP_032199 (mouse). The glucocorticoid receptor may be encoded by a nucleotide sequence of NM_000176 (human) and NM_008173 (mouse).

In some embodiments, the steroid is one or more selected from among cortisol, hydrocortin, cortisone, prednisolone, methyl prednisolone, triamcinolone, triamcinolone acetonide, paramethasone, dexamethasone, betamethasone, hexoestrol, methimazole, fluocinonide, fluocinolone acetonide, fluorometholone, beclometasone dipropionate, estriol, diflorasone diacetate, diflucortolone valerate, and difluprednate. Examples of medical conditions that may potentially be treated with corticosteroids includes allergy and respiratory conditions, such as asthma, chronic obstructive pulmonary disease (COPD), allergic rhinitis, atopic dermatitis, hives, angioedema, anaphylaxis, food allergies, drug allergies, nasal polyps, hypersensitivity pneumonitis, sarcoidosis, eosinophilic pneumonia and other pneumonias, and interstitial lung disease. Other conditions include dermatological conditions (e.g., pemphigus vulgaris, contact dermatitis), endocrine conditions (e.g., Addison's disease, adrenal insufficiency, congenital adrenal hyperplasia), gastroenterological conditions (e.g., ulcerative colitis, Crohn's disease, autoimmune hepatitis), hematological conditions (e.g., lymphoma, leukemia, hemolytic anemia, idiopathic thrombocytopenic purpura, multiple myeloma), rheumatological/immunological conditions (e.g., rheumatoid arthritis, systemic lupus erythematosus, polymyalgia rheumatic, polymyositis, dermatomyositis, polyarteritis, vasculitis), ophthalmological conditions (e.g., uveitis, optic neuritis, keratoconjunctivitis), and other conditions, such as multiple sclerosis relapses, organ transplant rejection, nephrotic syndrome, chronic hepatitis (flare ups), cerebral edema, IgG4-related diseases, prostate cancer, tendinosis, and lichen planus.

In some embodiments, the condition is an autoimmune disorder, such as rheumatoid arthritis, lupus erythematosus, or multiple sclerosis. In some embodiments, the condition is an inflammatory disorder such as atopic dermatitis, chronic obstructive pulmonary disease, or asthma.

The condition may be of any severity (e.g., mild, moderate, or severe), and may be acute or chronic.

In some embodiments, the condition is selected from among nephrotic syndrome, myasthenia gravis, lupus nephritis, cerebritis, dermatopolymyositis, temporal arteritis, immune hemolytic anemia, sarcoidosis, and systemic vasculitides.

The condition may be a cancer. The cancer may be a solid tumor such as breast cancer or prostate cancer, or a hematologic malignancy. In some embodiments, the cancer is acute lymphocytic leukemia, Hodgkin's lymphoma, non-Hodgkin's lymphoma, myeloma, or chronic lymphocytic leukemia.

In some embodiments, the individual has a dermatologic condition and the steroid therapy is topical steroid therapy. In some embodiments, the individual has a respiratory or lung disorder, and the steroid therapy is an inhaled steroid therapy. In some embodiments, the individual has an autoimmune disease, and the steroid therapy is a systemic steroid therapy.

The alternative non-steroidal therapy may be any non-steroid agent (i.e., non-steroid and not a steroid derivative) or non-steroidal treatment, such as a non-steroid chemical compound, radiation therapy, cryotherapy, surgery, or a combination of two or more of the foregoing. In some embodiments, the non-steroid alternative is a non-steroid anti-inflammatory agent, or non-steroid immune-suppressive agent, or non-steroid immunomodulator. Examples of conditions and corresponding steroid therapies and some alternative non-steroid therapies are shown in Table 1, below.

TABLE 1

| Condition | Steroid Therapies | Non-Steroid Alternatives |
|---|---|---|
| Rheumatoid arthritis | Prednisone, hydrocortisone, | methotrexate, sulfasalazine, |
| | prednisolone, | leflunomide, (Hydroxy-) |
| | dexamethasone, | chloroquine, tofacitinib, |
| | methylprednisolone, | baricitinib, etanercept, |
| | triamcinolone, | infliximab, adalimumab, |
| | betamethasone | golimumab, certolizumab, |
| | | tocilizumab, sarilumab, |
| | | abatacept, rituximab |
| Lupus erythematosus | Prednisone, prednisolone, | belimumab, tabalumab, |
| | methylprednisolone, | blisibimod, atacicept, |
| | cortisone, hydrocortisone, | abetimus sodium, rituximab, |
| | | ocrelizumab, ofatumumab, |
| | | obinutuzumab, |
| | | epratuzumab, rontalizumab, |
| | | sifalimumab |
| | | Anifrolumab, tocilizumab, |
| | | sirukumab, eculizumab, |
| | | ruplizumab/toralizumab, |
| | | dapirolizumab, abatacept |
| Severe atopic dermatitis | Clobetasol, betamethasone, | topical calcineurin inhibitor, |
| | mometasone, fluticasone, | topical PDE4 inhibitor, |
| | methylprednisolone, | tofacitinib, dupilumab, |
| | hydrocortisone, | cyclosporine, phototherapy, |
| | flumethasone | methotrexate, |
| | | mycophenolate mofetil |
| | | (MMF), azathioprine, |
| | | upadacitinib, PF-04965842, |
| | | baricitinib, nemolizumab, |
| | | lebrikizumab, tralokinumab, |
| | | fezakinumab, ustekinumab |
| Multiple sclerosis | Methylprednisolone, | topical tocoretinate, |
| | prednisone, dexamethasone | copaxone natalizumab, |
| | | alemtuzumab, daclizumab, |
| | | mitoxantrone |
| | | teriflunomide, delayed- |
| | | release dimethyl fumarate |
| | | (DMF), fingolimod, |
| | | rituximab, ocrelizumab, |
| | | ofatumumab, laquinimod, |
| | | cladribine, siponimod, |
| | | ozanimod, transplantation of |
| | | autologous bone marrow, |
| | | opicinumab, MSC |
| | | engraftment, autologous |
| | | MSCs |
| Asthma | prednisone | albuterol, levalbuterol, |
| | | terbutaline, metaproterenol |
| | | pirbuterol, salmeterol, |
| | | formoterol, zafirlukast, |
| | | montelukast, zileuton |
| | | ipratropium, tiotropium, |
| | | aclidinium, umeclidinium |
| | | glycopyrronium, |
| | | omalizumab, |
| | | mepolizumab, reslizumab |
| | | benralizumab, dupilumab, |
| | | tezepelumab |

The steroid therapy under consideration as a treatment for the individual may be a single steroid, or may be a combination of two or more steroids that utilize the same or different cellular pathways, and the steroid therapy may include a single steroid as the active agent or a combination of two or more steroids that utilize the same or different cellular pathways.

Techniques for formulation and administration of drugs, including steroids and non-steroids may be found in the latest edition of “Remington's Pharmaceutical Sciences,” Mack Publishing Co., Easton, Pa., which is herein fully incorporated by reference.

The steroid therapy may include administration of a steroid prodrug. Suitable routes of administration for steroids and the pharmaceutical compositions containing them may, for example, include topical, local injection, oral, inhalation, rectal, transmucosal, especially transnasal, intestinal, or parenteral delivery, including intramuscular, subcutaneous, and intramedullary injections, as well as intrathecal, direct intraventricular, intravenous, intraperitoneal, intranasal, or intraocular injections. Generally, the expression level of a biomarker of the invention (any one, two, three, four, five, or all six of MCAM, WFDC1, IGF1, IGFBP2, EYA4, and RBM24) may be determined at the RNA or protein level as a relative expression level. In some embodiments, the determination comprises contacting the sample with selective reagents (i.e., capture probes), such as probes, primers or ligands, and thereby detecting the presence, or measuring the amount, of immunoglobulin (antibody or antigen-binding antibody fragment), polypeptide, or nucleic acids of interest originally in the sample. Optionally, a detection device of the invention may be used for this purpose. The capture probes may be disposed on (immobilized, deposited on, or otherwise associated with) a substrate as an array. The capture probes may be arranged on the substrate of the array in an organized (spatially arranged) or random fashion.

In some embodiments, the capture probe is an antibody, or antibody fragment that specifically binds an antigen of interest (an antigen-binding fragment). In some embodiments, the capture probe is at least an antigenic epitope of an antigen (preferably, the full-length antigen) that induces antibodies that specifically bind the antigenic epitope or antigen. In some embodiments, the capture probes are oligonucleotides that bind to nucleic acid sequences encoding the protein biomarkers of interest. In some embodiments, the capture probes comprise or consist of:
(a) antibodies, or antibody fragments, that specifically bind one or more antigens on one or more of the protein biomarkers MCAM, WFDC1, IGF1, IGFBP2, EYA4, and RBM24; or
(b) oligonucleotides that are partially or fully complementary to, and bind (hybridize) to, nucleic acid sequences encoding one or more of the biomarkers MCAM, WFDC1, IGF1, IGFBP2, EYA4, and RBM24.

In some embodiments, the array comprises or consists of:
(a) antibodies, or antibody fragments, that specifically bind antigens on one, two, three, four, five, or all six of the protein biomarkers; or
(b) oligonucleotides that bind to nucleic acid sequences encoding one, two, three, four, five, or all six of the biomarkers.

Optionally, the arrays further include capture probes directed at other targets (e.g., different proteins or nucleic acids encoding different proteins, i.e., other than the six biomarkers). Alternatively, in some embodiments, arrays do not include captures probes for any other targets. In some embodiments, the arrays include capture probes directed to less than 500 targets in total (including any one, two, three, four, five, or all six of the biomarkers of the invention). In some embodiments, the arrays include capture probes directed to less than 100 targets in total (including any one, two, three, four, five, or all six of the biomarkers of the invention). In some embodiments, the arrays include capture probes directed to less than 50 targets in total (including any one, two, three, four, five, or all six of the biomarkers of the invention).

In some embodiments, the arrays have capture probes that target no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, or 100 molecular species in total.

The substrate bearing the capture probes is contacted with a sample from an individual that potentially contains the target binding partner of the capture probes (e.g., antibodies, antigens, or nucleic acid sequences (DNA or mRNA) encoding the polypeptides). Contacting may be performed in any suitable device. The substrate may be, for example, a plate, microtiter dish, test tube, well, glass, polymer, membrane, column, and so forth. In specific embodiments, the contacting is performed on a substrate coated with the capture probes, such as a nucleic acid array, protein array, antibody array, or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a chip, a gel, etc. The contacting may be made under any condition suitable for a detectable complex, such as a nucleic acid hybrid or an antibody-antigen complex, to be formed between the capture probe and the nucleic acids or polypeptides of the sample.

The invention also includes kits for the detection of two or more target antigens of the invention. In one embodiment, a kit of the invention comprises, in one or more separate containers, two or more capture probes of the invention. Optionally, the two or more capture probes are attached to a substrate. The kits may include one or more arrays of the invention. Kits of the invention can also optionally comprise additional reagents. Containers in a kit of the invention can be composed of any suitable material, such as glass or plastic. In one embodiment, a kit of the invention further comprises positive or negative controls or standards that the assayed sample can be compared to. In one embodiment, a kit of the invention can optionally comprise instructions pertaining to the use of the reagents and/or methods of the invention, packaging materials, sample diluents, buffers, wash reagents, and/or additional containers.

The arrays and kits of the invention may be used to carry out methods of the invention. The names, Universal Protein resource (UniProt) Reference Sequence Accession numbers, canonical nucleic acid sequences, and canonical amino acid sequences of the biomarkers of the invention are provided herein. Numeric sequence identifiers assigned to amino acid sequences representing embodiments of these biomarkers are as follows: MCAM amino acid sequence (SEQ ID NO:1), WFDC1 amino acid sequence (SEQ ID NO:2), IGF1 amino acid sequence (SEQ ID NO:3), IGFBP2 amino acid sequence (SEQ ID NO:4), EYA4 amino acid sequence (SEQ ID NO:5), and RBM24 amino acid sequence (SEQ ID NO:6), MCAM nucleic acid sequence (SEQ ID NO:7), WFDC1 nucleic acid sequence (SEQ ID NO:8), IGF1 nucleic acid sequence (SEQ ID NO:9), IGFBP2 nucleic acid sequence (SEQ ID NO:10), EYA4 nucleic acid sequence (SEQ ID NO:11), and RBM24 nucleic acid sequence (SEQ ID NO:12).

It should be understood that the biomarkers used in the subject invention also include variants of nucleic acid sequences of SEQ ID Nos:7-12, variant polypeptides encoded by those nucleic acid sequences, and variant polypeptides of SEQ ID NOs:1-6 including isoforms of these nucleic acid sequences and polypeptides. Preferably, the nucleic acid sequences encode functional polypeptides (functional versions of the recited polypeptide biomarkers). Variant sequences include those sequences wherein one or more nucleotides or amino acids of the sequence have been substituted, deleted, and/or inserted. Amino acids can be generally categorized in the following classes: non-polar, uncharged polar, basic, and acidic.

Conservative substitutions whereby a polypeptide having an amino acid of one class is replaced with another amino acid of the same class fall within the scope of the subject invention so long as the polypeptide having the substitution still retains substantially the same functional activity as the polypeptide that does not have the substitution. Polynucleotides encoding a polypeptide having one or more amino acid substitutions in the sequence are contemplated within the scope of the present invention.

Polynucleotides and polypeptides contemplated within the scope of the subject invention can also be defined in terms of more particular identity and/or similarity ranges with those sequences of the invention specifically exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein (e.g., compared to a sequence of SEQ ID NOs:1-12, or compared to a sequence encoded by SEQ ID NOs:1-12). Unless otherwise specified, as used herein, percent sequence identity and/or similarity of two sequences can be determined using the algorithm of Karlin and Altschul (1990) (“Methods for Assessing the Statistical Significance of Molecular Sequence Features by Using General Scoring Schemes” Proc. Natl. Acad. Sci. USA 87:2264-2268 (1990)), modified as in Karlin and Altschul (1993) (“Applications and Statistics for Multiple High-Scoring Segments in Molecular Sequences” Proc. Natl. Acad. Sci. USA 90:5873-5877 (1993)). Such an algorithm is incorporated into the NBLAST and XBLAST programs of Altschul et al. (1997) (“Gapped BLAST and PSI-BLAST: A New Generation of Protein Database Search Programs” Nucl. Acids Res. 25:3389-3402 (1997)). BLAST searches can be performed with the NBLAST program, score=100, word length=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

The subject invention also contemplates the use of those polynucleotide molecules having sequences which are sufficiently homologous with the polynucleotide sequences exemplified herein so as to permit hybridization with that sequence under standard stringent conditions and standard methods (Maniatis et al., 1982). As used herein, “stringent” conditions for hybridization refers to conditions wherein hybridization is typically carried out overnight at 20-25 C below the melting temperature (Tm) of the DNA hybrid in 6× SSPE, 5x Denhardt's solution, 0.1% SDS, 0.1 mg/ml denatured DNA. The melting temperature, Tm, is described by the following formula (Beltz et al., 1983):

Tm=81.5 C+16.6 Log[Na+]+0.41(%G+C)−0.61(% formamide)−600/length of duplex in base pairs.

Washes are typically carried out as follows:
(1) Twice at room temperature for 15 minutes in 1× SSPE, 0.1% SDS (low stringency wash).
(2) Once at Tm-20 C for 15 minutes in 0.2× SSPE, 0.1% SDS (moderate stringency wash).

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the

DNA strand that is transcribed. The polynucleotide sequences also include both full-length sequences as well as shorter sequences derived from the full-length sequences. Allelic variations of the exemplified sequences also fall within the scope of the subject invention. The polynucleotide sequence includes both the sense and antisense strands either as individual strands or in the duplex.

As used herein, the term “bind” refers to any physical attachment or close association, which may be permanent or temporary. The binding can result from hydrogen bonding, hydrophobic forces, van der Waals forces, covalent, or ionic bonding, for example. For example, the binding may be an antigen-antibody reaction, such as between an epitope on a protein biomarker of the invention and an antibody. Binding may also be hybridization at various stringencies through standard Watson and Crick type base-pairing, as between an oligonucleotide and a nucleic acid sequence encoding a biomarker of the invention.

As used herein, the term “sample” refers to a composition (e.g., biological composition) that potentially contains the target molecules (e.g., target proteins, target antigens, nucleic acid molecules, etc.) with which the capture probes are contacted. Thus, a sample potentially contains the target binding partner of capture probes (e.g., proteins, or nucleic acid sequences (DNA or mRNA) encoding the proteins). Samples may be removed from the body of a subject using any method or technique. For example, blood or other fluid samples may be removed using a syringe or needle. A swab may be used to remove endothelium cells. Other samples may be removed by biopsy or tissue section.

Examples of such samples include fluids such as blood (e.g., peripheral blood), whole blood, plasma, serum, saliva, urine, cerebrospinal fluid, seminal fluid, and other body fluid samples, as well as biopsies, organs, tissues, or cell samples. The sample may be treated prior to its use, e.g., in order to render target molecules accessible or more accessible (for example, target nucleic acids if nucleic acids levels such as RNA are to be measured). In some embodiments, the sample is a sample of a scar or keloid.

The sample may be a cellular sample (samples of intact cells, e.g., a cytology sample) or non-cellular sample. One or more samples may be obtained from a subject by techniques known in the art, such as biopsy. The type of biopsy utilized is dependent upon the anatomical location from which the sample is to be obtained. Methods for collecting various body samples are known in the art. Examples include fine needle aspiration (FSA), excisional biopsy, incisional biopsy, and punch biopsy. Samples may be transferred to a glass slide for viewing under magnification. Fixative and staining solutions may be applied to the cells on the slide for preserving the specimen and/or for facilitating examination. It should be understood that the methods of the invention may include a step in which a sample is obtained directly from an individual; alternatively, a sample may be obtained or otherwise provided, e.g., by a third party.

A sample may be taken from an individual having or suspected of having a condition. A sample may also comprise proteins isolated from a tissue or cell sample from a subject. In certain aspects, the sample can be, but is not limited to tissue (e.g., biopsy, particularly fine needle biopsy, excision, or punch biopsy), blood, serum, plasma.

In some embodiments, the sample is a cell sample that includes one or more cell types selected from among fibroblasts, epithelial cells, keratinocytes, and melanocytes. In some embodiments, the sample is a cell sample, and cells of the cell sample include normal cells. In some embodiments, the sample is a cell sample, and cells of the cell sample include abnormal or diseased cells. In some embodiments, the sample is a cell sample, and the cell sample includes cells of a keloid. In some embodiments, the sample is a cell sample, and the cell sample includes buccal cells, or cells of a surgical biopsy.

As indicated above, the invention includes an array comprising capture probes disposed on a substrate, in which the capture probes specifically bind (1) antigens (proteins) representing biomarkers of the invention, or (3) nucleic acid molecules encoding such proteins (see, for example, Berton P. and Snyder M., “Advances in functional protein microarray technology,” FEBS J, 2005; 272(21):5400-5411; Wingren C. and Borrebaeck C.A., “Antibody microarrys: current status and key technological advances,” OMICS, 2006, 10(3):411-427; Zhu H. and Snyder M., Curr. Opin. Chem. Biol., 2003, 7(1):55-63; Bussow K. et al., “Protein Array Technology: Potential Use in Medical Diagnostics,” Am. J. Pharmaceogenomics, 2001, 1(1):1-7). Thus, for example, the array can be an antibody array (with antibodies or antigen-binding antibody fragments disposed on the substrate), or a nucleic acid array (with oligonucleotides disposed on the substrate, in which the oligonucleotides are partially or fully complementary with nucleic acid sequences encoding the proteins).

The substrate may be any solid or semi-solid support for supporting the capture probes, such as a particle (e.g., magnetic or latex particle), a microtiter multi-well plate (e.g., 96-well, 384-well, 1536-well, etc.), a bead, a slide, a filter, a chip, a membrane, a cuvette, or a reaction vessel. The capture probes may be manufactured synthetically directly on the substrate or be produced and subsequently immobilized or otherwise attached to the substrate using standard technologies such as pin-based spotting, liquid microdispensing, adsorption to charged or hydrophobic surfaces, covalent cross-linking or specific binding via tags (e.g., nickel chelating or streptavidin coated surfaces for plasmon resonance measurements). In the arrays of the invention, the capture probes can be in ordered arrangements on the substrates, or be randomly disposed, and can be of various densities.

Detectable labels that can be used with the present invention include, but are not limited to, enzymes, radioisotopes, chemiluminescent and bioluminescent reagents, and fluorescent moieties. Enzymes that can be used include but are not limited to lucerifase, beta-galactosidase, acetylcholinesterase, horseradish peroxidase, glucose -6-phosphate dehydrogenase, and alkaline phosphatase. If the detectable label is an enzyme, then a suitable substrate that can be acted upon by the enzyme can be used for detection and measurement of enzyme activity. In one embodiment, if the detectable label is a peroxidase, the substrate can be hydrogen peroxide (H₂O₂) and 3-3′ diaminobenzidine or 4-chloro-1-naphthol and the like. Other substrates suitable for use with other enzymes are well known in the art. An example of a luminescent material includes luminol. Examples of bioluminescent materials include, but are not limited to, luciferin, green fluorescent protein (GFP), enhanced GFP (Yang et al., 1996), and aequorin. Fluorescent moieties include, but are not limited to, umbelliferone, fluorescein, fluorescein isothiocyanate, Cascade Blue, rhodamine, dichlorotriazinylamine fluorescein, dansyl chloride, Texas Red, Oregon Green, cyanines (e.g., CY2, CY3, and CY5), allophycocyanine, or phycoerythrin. Isotopes that can be used include, but are not limited to, ¹²⁵I, ¹⁴C, ³⁵S, and ³H.

In embodiments of the various methods of the invention, the expression level of the one or more genes may be the level of mRNA or the level of protein. The expression level of one or more biomarkers may be determined using a nucleic acid measurement technique comprising one or more of: polymerase chain reaction (PCR, such as quantitative Reverse Transcription PCR (qRT-PCR) and digital PCR), microarray analysis, whole transcriptome shotgun sequencing (RNA-seq), and direct multiplexed gene expression analysis. The expression level one or more biomarkers may be determined using a protein measurement technique comprising one or more of: a spectrometry method (e.g., high-performance liquid chromatography (HPLC), and liquid chromatography-mass spectrometry) or an immuno-based method (e.g., enzyme-linked immunosorbent assay (ELISA), protein immunoprecipitation, immunoelectrophoresis, Western blot, and protein immunostaining).

### Antibodies

Antibodies contemplated for use in the present invention can be in any of a variety of forms, including a whole immunoglobulin, an antibody fragment such as Fv, Fab, and similar fragments, a single chain antibody that includes the variable domain complementarity determining regions (CDR), and the like forms, all of which fall under the broad term “antibody,” as used herein. Antibodies useful in the arrays, kits, and methods of the present invention can be monoclonal or polyclonal antibodies, and can be from any source including, but not limited to, mouse, rabbit, goat, rat, or human. Antibodies of the invention can be conjugated to a detectable label, such as, for example, a fluorescent moiety. In one embodiment of the present invention, a detectable label can be directly bound to an antibody that binds to an epitope on a biomarker of the invention (or to another antibody that binds the epitope). If the detectable label is to be directly bound, the label may comprise a functional group which is capable of binding to the antibody used with the invention. Alternatively, the detectable label may be indirectly bound, for example, using an avidin-biotin or streptavidin-biotin bridge wherein the avidin or biotin is labeled with a detectable label. In one embodiment, an antibody of the invention is conjugated with avidin and the detectable label is conjugated with biotin.

The term “antibody fragment”, “antigen-binding fragment”, or “antigen-binding antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments, which are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

Antibody fragments can retain an ability to selectively bind with the antigen or analyte and are defined as follows:
(1) Fab is the fragment that contains a monovalent antigen-binding fragment of an antibody molecule. A Fab fragment can be produced by digestion of whole antibody with the enzyme papain to yield an intact light chain and a portion of one heavy chain.
(2) Fab′ is the fragment of an antibody molecule can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.
(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.
(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.
(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269 315 (1994). Antibodies specific for protein biomarkers of the invention that are used in the methods, arrays, and kits of the invention may be obtained from scientific or commercial sources. Alternatively, isolated native polypeptides or recombinant polypeptides may be utilized to prepare antibodies, monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladne et al., U.S. Pat. No. 4,946,778), or a chimeric antibody, for example, an antibody which contains the binding specificity of a murine antibody, but in which the remaining portions are of human origin. Antibodies, including monoclonal and polyclonal antibodies, fragments and chimeras, may be prepared using methods known to those skilled in the art. In some embodiments, antibodies used in the methods of the invention are reactive against antigens of the invention if they bind with a Kₐ of greater than or equal to 107 M. In a sandwich immunoassay of the invention, mouse polyclonal antibodies and rabbit polyclonal antibodies can be utilized, for example.

In order to produce monoclonal antibodies, a host mammal can be inoculated with a protein or peptide representing a protein biomarker of the invention and then boosted. Spleens are collected from inoculated mammals a few days after the final boost. Cell suspensions from the spleens are fused with a tumor cell in accordance with the general method described by Kohler and Milstein (Nature, 1975, 256:495-497). In order to be useful, a peptide fragment must contain sufficient amino acid residues to define the epitope of the biomarker molecule being detected.

If the fragment is too short to be immunogenic, it may be conjugated to a carrier molecule. Some suitable carrier molecules include keyhole limpet hemocyanin and bovine serum albumin. Conjugation may be carried out by methods known in the art. One such method is to combine a cysteine residue of the fragment with a cysteine residue on the carrier molecule. The peptide fragments may be synthesized by methods known in the art. Some suitable methods are described by Stuart and Young in “Solid Phase Peptide Synthesis,” Second Edition, Pierce Chemical Company (1984).

Purification of the antibodies or fragments can be accomplished by a variety of methods known to those skilled in the art including, precipitation by ammonium sulfate or sodium sulfate followed by dialysis against saline, ion exchange chromatography, affinity or immunoaffinity chromatography as well as gel filtration, zone electrophoresis, etc. (Goding in, Monoclonal Antibodies: Principles and Practice, 2d ed., pp. 104-126, Orlando, Fla., Academic Press). It is preferable to use purified antibodies or purified fragments of the antibodies having at least a portion of an antigenic binding region, including such as Fv, F(ab′)₂, Fab fragments (Harlow and Lane, 1988, Antibody Cold Spring Harbor) for the detection of the biomarker proteins in the samples of subjects.

For use in detection, the purified antibodies can be covalently attached, either directly or via linker, to a compound which serves as a reporter group to permit detection of the presence of the antigen. A variety of different types of substances can serve as the reporter group, including but not limited to enzymes, dyes, radioactive metal and non-metal isotopes, fluorogenic compounds, fluorescent compounds, etc. Methods for preparation of antibody conjugates of the antibodies (or fragments thereof) of the invention useful for detection, monitoring are described in U.S. Pat. Nos. 4,671,958; 4,741,900 and 4,867,973.

In one aspect of the invention, preferred binding epitopes may be identified from a known gene sequence and its encoded amino acid sequence and used to generate antibodies to an antigen of a protein biomarker with high binding affinity. Also, identification of binding epitopes on the antigen can be used in the design and construction of preferred antibodies. For example, a DNA encoding a preferred epitope on a biomarker protein may be recombinantly expressed and used to select an antibody which binds selectively to that epitope. The selected antibodies then are exposed to the sample under conditions sufficient to allow specific binding of the antibody to the specific binding epitope on the antigen and the amount of complex formed then detected. Specific antibody methodologies are well understood and described in the literature. A more detailed description of their preparation can be found, for example, in Practical Immunology, Butt, W. R., ed., Marcel Dekker, New York, 1984.

### Protein Targets (Protein Binding Assays)

Antibodies specifically reactive with the antigens of protein biomarkers disclosed herein or derivatives, such as enzyme conjugates or labeled derivatives, may be used to the detect antigens in various samples, for example they may be used in any known immunoassays which rely on the binding interaction between an antigenic determinant of a protein and the antibodies. Examples of such assays are radioimmunoassays, enzyme immunoassay (e.g., ELISA), immunofluorescence, immunoprecipitation, latex agglutination, hemagglutination, and histochemical tests.

An antibody specific for an antigen of a biomarker in the invention can be labeled with a detectable substance and localized in biological samples based upon the presence of the detectable substance. Examples of detectable substances include, but are not limited to, the following radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinestease), biotinyl groups (which can be detected by marked avidin, e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). Indirect methods may also be employed in which the primary antigen-antibody reaction is amplified by the introduction of a second antibody, having specificity for the antibody reactive against the biomarker protein. By way of example, if the antibody having specificity against a biomarker protein is a rabbit IgG antibody, the second antibody may be goat anti-rabbit gamma-globulin labeled with a detectable substance.

Methods for conjugating or labeling the antibodies discussed above may be readily accomplished by one of ordinary skill in the art. (See, for example, Imman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem., 1988, 171:1-32, regarding methods for conjugating or labeling the antibodies with an enzyme or ligand binding partner).

Time-resolved fluorometry may be used to detect a signal. For example, the method described in Christopoulos T. K. and Diamandis E. P., Anal. Chem., 1992:64:342-346 may be used with a conventional time-resolved fluorometer.

Therefore, in accordance with an embodiment of the invention, a method is provided wherein an antibody to an antigen of a biomarker of the invention is labeled with an enzyme, a substrate for the enzyme is added wherein the substrate is selected so that the substrate, or a reaction product of the enzyme and substrate, forms fluorescent complexes with a lanthanide metal. A lanthanide metal is added and the antigen is quantitated in the sample by measuring fluorescence of the fluorescent complexes. The antibodies specific for the antigen may be directly or indirectly labeled with an enzyme. Enzymes are selected based on the ability of a substrate of the enzyme, or a reaction product of the enzyme and substrate, to complex with lanthanide metals such as europium and terbium. Examples of suitable enzymes include alkaline phosphatase and beta-galactosidase. Preferably, the enzyme is alkaline phosphatase. The antibodies may also be indirectly labeled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. Preferably the antibodies are biotinylated, and the enzyme is coupled to streptavidin.

In an embodiment of the invention, antibody bound to an antigen of a biomarker of the invention in a sample is detected by adding a substrate for the enzyme. The substrate is selected so that in the presence of a lanthanide metal (e.g., europium, terbium, samarium, and dysprosium, preferably europium and terbium), the substrate or a reaction product of the enzyme and substrate, forms a fluorescent complex with the lanthanide metal. Examples of enzymes and substrates for enzymes that provide such fluorescent complexes are described in U.S. Pat. No. 5,312,922 to Diamandis. By way of example, when the antibody is directly or indirectly labeled with alkaline phosphatase, the substrate employed in the method may be 4 -methylumbeliferyl phosphate, or 5-fluorpsalicyl phosphate. The fluorescence intensity of the complexes is typically measured using a time-resolved fluorometer, e.g., a CyberFluor 615 Immoanalyzer (Nordion International, Kanata Ontario).

The antibody (or antibody fragment) specific for the antigen may be immobilized on a substrate. Examples of suitable substrates are agarose, cellulose, dextran, Sephadex, Sepharose, liposomes, carboxymethyl cellulose polystyrene, filter paper, ion-exchange resin, plastic film, plastic tube, glass beads, polyamine-methyl vinyl ether-maleic acid copolymer, amino acid copolymer, ethylene-maleic acid copolymer, nylon, silk, etc. The substrate may be in the shape of, for example, a tube, test plate, well, beads, disc, chip, sphere, etc. The immobilized antibody may be prepared by reacting the material with a suitable insoluble carrier using known chemical or physical methods, for example, cyanogen bromide coupling.

In accordance with an embodiment, the present invention provides a mode for determining the presence and, preferably, the abundance of antigen of protein biomarkers, or nucleic acid sequences encoding the biomarker proteins, in an appropriate sample, such as a keloid sample or fluid sample, by measuring the antigen or nucleic acids. The antigens and nucleic acids can be removed from tissue using methods known in the art. It will be evident to a skilled artisan that a variety of immunoassay methods can be used to measure these biomolecules. In general, an immunoassay method may be competitive or noncompetitive. Competitive methods typically employ an immobilized or immobilizable antibody to the antigen and a labeled form of the antigen. Sample antigen and labeled antigen compete for binding to the antibody. After separation of the resulting labeled antigen that has become bound to antibody (bound fraction) from that which has remained unbound (unbound fraction), the amount of the label in either bound or unbound fraction is measured and may be correlated with the amount of antigen in the biological sample in any conventional manner, e.g., by comparison to a standard curve.

Preferably, a noncompetitive method is used for the determination of one or more antigen of protein biomarkers of the invention, with the most common method being the “sandwich” method. In this assay, two anti-antigen antibodies, such as two anti-tumor antigen antibodies, are employed. One of the antibodies is directly or indirectly labeled (also referred to as the “detection antibody”) and the other is immobilized or immobilizable (also referred to as the “capture antibody”). The capture and detection antibodies can be contacted simultaneously or sequentially with the biological sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally, it is measured in the capture antibody phase since it comprises the antigen bound by (“sandwiched” between) the capture and detection antibodies.

In a typical two-site immunometric assay for an antigen, one or both of the capture and detection antibodies are polyclonal antibodies. The label used in the detection antibody can be selected from any of those known conventionally in the art. As with other embodiments of the protein detection assay, the label can be an enzyme or a chemiluminescent moiety, for example, or a radioactive isotope, a fluorophore, a detectable ligand (e.g., detectable by a secondary binding by a labeled binding partner for the ligand), and the like. Preferably, the antibody is labeled with an enzyme that is detected by adding a substrate that is selected so that a reaction product of the enzyme and substrate forms fluorescent complexes. The capture antibody is selected so that it provides a mode for being separated from the remainder of the test mixture.

Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody can comprise an antibody covalently or noncovalently attached to a solid phase (substrate) such as a magnetic particle, a latex particle, a microtiter multi-well plate, a bead, a cuvette, chip, slide, or other reaction vessel. An example of an immobilizable capture antibody is an antibody that has been chemically modified with a ligand moiety, e.g., a hapten, biotin, or the like, and that can be subsequently immobilized by contact with an immobilized form of a binding partner for the ligand, e.g., an antibody, avidin, or the like. In an embodiment, the capture antibody can be immobilized using a species specific antibody for the capture antibody that is bound to the solid phase.

A particular sandwich immunoassay method of the invention employs two antibodies reactive against an antigen of a protein biomarker of the invention, a second antibody having specificity against an antibody reactive against the antigen labeled with an enzymatic label, and a fluorogenic substrate for the enzyme. In an embodiment, the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb³⁺ fluorescence in a time-resolved mode. Fluorescence intensity is typically measured using a time-resolved fluorometry as described herein.

The above-described immunoassay methods and formats are intended to be exemplary and are not limiting since, in general, it will be understood that any immunoassay method or format can be used in the present invention.

The detection methods, arrays, and kits of the invention can utilize nanowire sensor technology (Zhen et al., Nature Biotechnology, 2005, 23(10):1294-1301; Lieber et al., Anal. Chem., 2006, 78(13):4260-4269, which are incorporated herein by reference) or microcantilever technology (Lee et al., Biosens. Bioelectron, 2005, 20(10):2157-2162; Wee et al., Biosens. Bioelectron., 2005, 20(10):1932-1938; Campbell and Mutharasan, Biosens. Bioelectron., 2005, 21(3):462-473; Campbell and Mutharasan, Biosens. Bioelectron., 2005, 21(4):597-607; Hwang et al., Lab Chip, 2004, 4(6):547-552; Mukhopadhyay et al., Nano. Lett., 2005, 5(12):2835-2388, which are incorporated herein by reference) for detection of one or more antigens or nucleic acid sequences in samples. In addition, Huang et al. describe a prostate specific antigen immunoassay on a commercially available surface plasmon resonance biosensor (Biosens. Bioelectron., 2005, 21(3):483-490) which may be adapted for detection of one or more antigens of protein biomarkers of the invention. High-sensitivity miniaturized immunoassays may also be utilized for detection of the antigens (Cesaro-Tadic et al., Lab Chip, 2004, 4(6):563-569; Zimmerman et al., Biomed. Microdevices, 2005, 7(2):99-110).

The level of one or more of the biomarkers may be measured using a rapid antibody test, which can be performed at the point of care in minutes. Rapid antibody tests are particularly useful for quickly measuring the level of biomarker proteins in less invasive samples at the point of care, such as body fluids (e.g., whole blood, serum, plasma, saliva, urine, etc.).

### Nucleic Acid Targets

Nucleic acids including naturally occurring nucleic acids, oligonucleotides, antisense oligonucleotides, and synthetic oligonucleotides that hybridize to target nucleic acids within target genes or transcripts (e.g., encoding biomarker polypeptides), are useful as agents to detect the presence of nucleic acids encoding the protein biomarkers in biological samples of individuals, such as keloid samples. The present invention contemplates the use of nucleic acid sequences corresponding to the coding sequence of the protein biomarkers and to the complementary sequence thereof, as well as sequences complementary to the transcript sequences occurring further upstream or downstream from the coding sequence (e.g., sequences contained in, or extending into, the 5′ and 3′ untranslated regions) for use as agents for detecting the expression of protein biomarkers in samples of individuals.

The preferred oligonucleotides for detecting the presence of protein biomarkers in samples are those that are complementary to at least part of the cDNA sequence encoding the proteins. These complementary sequences are also known in the art as “antisense” sequences. These oligonucleotides may be oligoribonucleotides or oligodeoxyribonucleotides. In addition, oligonucleotides may be natural oligomers composed of the biologically significant nucleotides, i.e., A (adenine), dA (deoxyadenine), G (guanine), dG (deoxyguanine), C (cytosine), dC (deoxycytosine), T (thymine) and U (uracil), or modified oligonucleotide species, substituting, for example, a methyl group or a sulfur atom for a phosphate oxygen in the inter-nucleotide phosphodiester linkage. Additionally, these nucleotides themselves, and/or the ribose moieties may be modified.

The oligonucleotides may be synthesized chemically, using any of the known chemical oligonucleotide synthesis methods well described in the art. For example, the oligonucleotides can be prepared by using any of the commercially available, automated nucleic acid synthesizers. Alternatively, the oligonucleotides may be created by standard recombinant DNA techniques, for example, inducing transcription of the noncoding strand. The DNA sequence encoding the biomarker polypeptide may be inverted in a recombinant DNA system, e.g., inserted in reverse orientation downstream of a suitable promoter, such that the noncoding strand now is transcribed.

Although any length oligonucleotide may be utilized to hybridize to a target nucleic acid within biomarker genes or transcripts (e.g., to a nucleic acid encoding a protein biomarker), oligonucleotides typically within the range of 8-100 nucleotides are preferred. Most preferable oligonucleotides for use in detecting biomarker genes or transcripts in biological samples are those within the range of 15-50 nucleotides.

In some embodiments, the substrate (e.g., solid support) of the array of the invention has oligonucleotides to no more than 500 distinct targets attached to it. In some embodiments, the substrate has oligonucleotides to no more than 100 distinct targets attached to it. In some embodiments, the substrate has oligonucleotides to no more than 50 distinct targets attached to it. In some embodiments, the substrate has oligonucleotides to no more than 20 distinct targets attached to it. In some embodiments, the substrate has oligonucleotides to no more than 10 distinct targets attached to it. In some embodiments, the substrate has oligonucleotides to no more than 5 distinct targets attached to it.

When referring to hybridization of one nucleic to another, “low stringency conditions” means in 10% formamide, 5× Denhart's solution, 6× SSPE, 0.2% SDS at 42° C., followed by washing in 1× SSPE, 0.2% SDS, at 50° C.; “moderate stringency conditions” means in 50% formamide, 5× Denhart's solution, 5× SSPE, 0.2% SDS at 42° C., followed by washing in 0.2× SSPE, 0.2% SDS, at 65° C.; and “high stringency conditions” means in 50% formamide, 5× Denhart's solution, 5× SSPE, 0.2% SDS at 42° C., followed by washing in 0.1× SSPE, and 0.1% SDS at 65° C. The phrase “stringent hybridization conditions” means low, moderate, or high stringency conditions.

The oligonucleotide selected for hybridizing to the nucleic acid molecule encoding the protein biomarker, whether synthesized chemically or by recombinant DNA technology, can be isolated and purified using standard techniques and then preferably labeled (e.g., with ³⁵S or ³²P) using standard labeling protocols. Oligonucleotides can be attached or immobilized to a suitable such as a solid or semi-solid support using methods known in the art.

The present invention also contemplates the use of oligonucleotide pairs (e.g., primers) in polymerize chain reactions (PCR) to detect the expression of biomarker encoding nucleic acid sequences in biological samples. The oligonucleotide pairs include a forward primer and a reverse primer.

The presence of antigen in a sample from a subject may be determined by nucleic acid hybridization, such as but not limited to Northern blot analysis, dot blotting, Southern blot analysis, fluorescence in situ hybridization (FISH), and PCR. Chromatography, preferably HPLC, and other known assays may also be used to determine messenger RNA levels of antigens in a sample.

In one aspect, the present invention contemplates the use of nucleic acids as agents (oligonucleotides) for detecting biomarkers in samples, wherein the nucleic acids are labeled. The oligonucleotides may be labeled with a radioactive label, a fluorescent label, an enzyme, a chemiluminescent tag, a colorimetric tag or other labels or tags that are discussed above or that are known in the art.

In another aspect, the present invention contemplates the use of Northern blot analysis to detect the presence of biomarker mRNA in a sample. The first step of the analysis involves separating a sample containing antigen-encoding nucleic acid by gel electrophoresis. The dispersed nucleic acids are then transferred to a nitrocellulose filter or another filter. Subsequently, the labeled oligonucleotide is exposed to the filter under suitable hybridizing conditions, e.g., 50% formamide, 5×SSPE, 2×Denhardt's solution, 0.1% SDS at 42° C., as described in Molecular Cloning: A Laboratory Manual, Maniatis et al. (1982, CSH Laboratory). Other useful procedures known in the art include solution hybridization, dot and slot RNA hybridization, and probe-based microarrays. Measuring the radioactivity of hybridized fragments, using standard procedures known in the art quantitates the amount of nucleic acid present in the sample of a subject.

Dot blotting involves applying samples containing the nucleic acid of interest to a membrane. The nucleic acid can be denatured before or after application to the membrane. The membrane is incubated with a labeled probe. Dot blot procedures are well known to the skilled artisan and are described more fully in U.S. Pat. Nos. 4,582,789 and 4,617,261, the disclosures of which are incorporated herein by reference.

Polymerase chain reaction (PCR) is a process for amplifying one or more target nucleic acid sequences present in a nucleic acid sample using primers and agents for polymerization and then detecting the amplified sequence. The extension product of one primer when hybridized to the other becomes a template for the production of the desired specific nucleic acid sequence, and vice versa, and the process is repeated as often as is necessary to produce the desired amount of the sequence. The skilled artisan to detect the presence of desired sequence (U.S. Pat. No. 4,683,195) routinely uses PCR.

A specific example of PCR that is routinely performed by the skilled artisan to detect desired sequences is reverse transcription PCR (RT-PCR; Saiki et al., Science, 1985, 230:1350; Scharf et al., Science, 1986, 233:1076). RT-PCR involves isolating total RNA from biological samples (tissues or fluid), denaturing the RNA in the presence of primers that recognize the desired nucleic acid sequence, using the primers and a reverse transcriptase enzyme to generate a cDNA copy of the RNA by reverse transcription, amplifying the cDNA by PCR using specific primers, and detecting the amplified cDNA by electrophoresis or other methods known to the skilled artisan.

In a preferred embodiment, the methods of detecting nucleic acids encoding protein biomarkers in samples of individuals include Northern blot analysis, dot blotting, Southern blot analysis, FISH, and PCR.

### Miscellaneous Assay Components and Platforms

The methods of the invention can be carried out on a substrate (e.g., solid or semi-solid support). The solid supports used may be those which are conventional for the purpose of assaying an analyte in a biological sample, and are typically constructed of materials such as cellulose, polysaccharide such as Sephadex, and the like, and may be partially surrounded by a housing for protection and/or handling of the solid support. The solid support can be rigid, semi-rigid, flexible, elastic (having shape-memory), etc., depending upon the desired application. Biomarkers of the invention can be detected in a sample in vivo or in vitro (ex vivo). When, according to an embodiment of the invention, the amount of protein biomarker in a sample is to be determined without removing the sample from the body (i.e., in vivo, such as with an indwelling catheter or probe), the support should be one which is harmless to the individual and may be in any form convenient for insertion into an appropriate part of the body. For example, the support may be a probe made of polytetrafluoroethylene, polystyrene or other rigid non-harmful plastic material and having a size and shape to enable it to be introduced into a subject. The selection of an appropriate inert support is within the competence of those skilled in the art, as are its dimensions for the intended purpose.

A contacting step made in determining biomarker levels in an assay (method) of the invention can involve contacting, combining, or mixing the biological sample and the support, such as a reaction vessel, microvessel, tube, microtube, well, multi-well plate, or other support. In an embodiment of the invention, the support to be contacted with the biological sample has an absorbent pad or membrane for lateral flow of the liquid medium to be assayed, such as those available from Millipore Corp. (Bedford, MA), including but not limited to Hi-Flow Plus™ membranes and membrane cards, and SureWick™ pad materials.

Arrays useful in carrying out the methods of the invention can be constructed in any form adapted for the intended use. Thus, in one embodiment, the device can be constructed as a disposable or reusable test strip or stick to be contacted with a sample for which the presence of protein (e.g., an epitope), nucleic acid sequence, or level thereof is to be determined. In another embodiment, the device can be constructed using art recognized micro-scale manufacturing techniques to produce needle-like embodiments capable of being implanted or injected into an anatomical site, such as a vein or artery, for indwelling diagnostic applications. In other embodiments, devices intended for repeated laboratory use can be constructed in the form of an elongated probe or catheter, for sampling of blood.

In some embodiments, the arrays of the invention comprise a solid support (such as a strip or dipstick), with a surface that functions as a lateral flow matrix defining a flow path for a biological sample such as a biological fluid.

Immunochromatographic assays, also known as lateral flow test strips or simply strip tests, for detecting various analytes of interest, have been known for some time, and may be used for detection of biomarkers of the invention. The benefits of lateral flow tests include a user-friendly format, rapid results, long-term stability over a wide range of climates, and relatively low cost to manufacture. These features make lateral flow tests ideal for applications involving home testing, rapid point of care testing, and testing in the field for various analytes. The principle behind the test is straightforward. Essentially, any ligand that can be bound to a visually detectable solid support, such as dyed microspheres, can be tested for, qualitatively, and in many cases even semi-quantitatively. For example, a one-step lateral flow immunostrip for the detection of free and total prostate specific antigen in serum is described in Fernandez-Sanchez et al. (J. Immuno. Methods, 2005, 307(1-2):1-12, which is incorporated herein by reference) and may be adapted for detection of one or more biomarkers of the invention in a biological sample.

Some of the more common immunochromatographic assays currently on the market are tests for pregnancy (as an over-the-counter (OTC) test kit), Strep throat, and Chlamydia. Many new tests for well-known antigens have been recently developed using the immunochromatographic assay method. For instance, the antigen for the most common cause of community acquired pneumonia has been known since 1917, but a simple assay was developed only recently, and this was done using this simple test strip method (Murdoch, D. R. et al. J Clin Microbiol, 2001, 39:3495-3498). Human immunodeficiency virus (HIV) has been detected rapidly in pooled blood using a similar assay (Soroka, S. D. et al. J Clin Virol, 2003, 20 27:90-96). A nitrocellulose membrane card has also been used to diagnose schistosomiasis by detecting the movement and binding of nanoparticles of carbon (van Dam, G. J. et al. J Clin Microbiol, 2004, 42:5458-5461).

The two common approaches to the immunochromatographic assay are the non-competitive (or direct) and competitive (or competitive inhibition) reaction schemes (TechNote #303, Rev. #001, 1999, Bangs Laboratories, Inc., Fishers, IN). The direct (double antibody sandwich) format is typically used when testing for larger analytes with multiple antigenic sites such as luteinizing hormone (LH), human chorionic gonadotropin (hCG), and HIV. In this instance, less than an excess of sample analyte is desired, so that some of the microspheres will not be captured at the capture line, and will continue to flow toward the second line of immobilized antibodies, the control zone. This control line uses species-specific anti-immunoglobulin antibodies, specific for the conjugate antibodies on the microspheres. Free antigen, if present, is introduced onto the device by adding sample (blood, etc.) onto a sample addition pad. Free antigen then binds to antibody-microsphere complexes. Antibody 1, specific for epitope 1 of sample antigen, is coupled to dye microspheres and dried onto the device. When sample is added, microsphere-antibody complex is rehydrated and carried to a capture zone and control lines by liquid. Antibody 2, specific for a second antigenic site (epitope 2) of sample antigen, is dried onto a membrane at the capture line. Antibody 3, a species-specific, anti-immunoglobulin antibody that will react with antibody 1, is dried onto the membrane at the control line. If antigen is present in the sample (i.e., a positive test), it will bind by its two antigenic sites, to both antibody 1 (conjugated to microspheres) and antibody 2 (dried onto membrane at the capture line). Antibody 1-coated microspheres are bound by antibody 3 at the control line, whether antigen is present or not. If antigen is not present in the sample (a negative test), microspheres pass the capture line without being trapped, but are caught by the control line.

The competitive reaction scheme is typically used when testing for small molecules with single antigenic determinants, which cannot bond to two antibodies simultaneously. As with double antibody sandwich assay, free antigen, if present is introduced onto the device by adding sample onto a sample pad. Free antigen present in the sample binds to an antibody-microsphere complex. Antibody 1 is specific for sample antigen and couple to dyed microspheres. An antigen-carrier molecule (typically BSA) conjugate is dried onto a membrane at the capture line. Antibody 2 (Ab2) is dried onto the membrane at the control line, and is a species-specific anti-immunoglobulin that will capture the reagent particles and confirm that the test is complete. If antigen is present in the sample (a positive test), antibody on microspheres (Ab 1) is already saturated with antigen from sample and, therefore, antigen conjugate bound at the capture line does not bind to it. Any microspheres not caught by the antigen carrier molecule can be caught by Ab2 on the control line. If antigen is not present in the sample (a negative test), antibody-coated dyed microspheres are allowed to be captured by antigen conjugate bound at the capture line.

Normally, the membranes used to hold the antibodies in place on these devices are made of primary hydrophobic materials, such as nitrocellulose. Both the microspheres used as the solid phase supports and the conjugate antibodies are hydrophobic, and their interaction with the membrane allows them to be effectively dried onto the membrane.

As used herein, the term “ELISA” includes an enzyme-linked immunoabsorbent assay that employs an antibody or antigen bound to a solid phase and an enzyme-antigen or enzyme-antibody conjugate to detect and quantify the amount of an antigen (e.g., biomarker of the invention) or antibody present in a sample. A description of the ELISA technique is found in Chapter 22 of the 4^{th} Edition of Basic and Clinical Immunology by D. P. Sites et al., 1982, published by Lange Medical Publications of Los Altos, Calif. and in U.S. Pat. Nos. 3,654,090; 3,850,752; and 4,016,043, the disclosures of which are herein incorporated by reference. ELISA is an assay that can be used to quantitate the amount of antigen, proteins, or other molecules of interest in a sample. In particular, ELISA can be carried out by attaching on a solid support (e.g., polyvinylchloride) an antibody specific for an antigen or protein of interest. Cell extract or other biological sample of interest such as blood can be added for formation of an antibody-antigen complex, and the extra, unbound sample is washed away. An enzyme-linked antibody, specific for a different site on the antigen is added. The support is washed to remove the unbound enzyme-linked second antibody. The enzyme-linked antibody can include, but is not limited to, alkaline phosphatase. The enzyme on the second antibody can convert an added colorless substrate into a colored product or can convert a non-fluorescent substrate into a fluorescent product. The ELISA-based assay method provided herein can be conducted in a single chamber or on an array of chambers and can be adapted for automated processes.

In these exemplary embodiments, the antibodies can be labeled with pairs of FRET dyes, bioluminescence resonance energy transfer (BRET) protein, fluorescent dye-quencher dye combinations, beta gal complementation assays protein fragments. The antibodies may participate in FRET, BRET, fluorescence quenching or beta-gal complementation to generate fluorescence, colorimetric or enhanced chemiluminescence (ECL) signals, for example.

These methods are routinely employed in the detection of antigen-specific antibody responses, and are well described in general immunology text books such as Immunology by Ivan Roitt, Jonathan Brostoff and David Male (London: Mosby, c1998. 5th ed. and Immunobiology: Immune System in Health and Disease/Charles A. Janeway and Paul Travers. Oxford: Blackwell Sci. Pub., 1994), the contents of which are herein incorporated by reference.

In some embodiments, the level of one or more of the biomarkers may be measured using a rapid antibody test, which may be performed at the point of care. In some embodiments, the rapid antibody is used to determine the level of one or more biomarker proteins in a body fluid sample (e.g., whole blood, serum, plasma, saliva, urine, etc.).

Each method disclosed and claimed herein can be used in combination with any other method disclosed or claimed herein, carried out concurrently or consecutively in any order.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and

Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. Eds. (1991) IRL Press)), each of which are incorporated herein by reference in their entirety.

### Further Definitions

As used herein, the terms “a” and “an” preceding an element or component of the invention are intended to be nonrestrictive regarding the number of instances, i.e., occurrences of the element or component. Therefore, “a” or “an” should be read to include one or at least one, and the singular word form of the element or component also includes the plural unless the number is obviously meant to be singular. For example, “a steroid” or “a test steroid” is inclusive of an individual steroid and a combination of two or more steroids.

As used herein, the term “active ingredient” refers to the agent accountable for the intended biological effect (e.g., a steroid agent, or non-steroid agent). As used herein, the phrases “physiologically acceptable carrier” and “pharmaceutically acceptable carrier,” which may be used interchangeably, refer to a carrier or a diluent that does not cause significant irritation to the subject and does not abrogate the biological activity and properties of the administered agent.

As used herein, the term “administration” is intended to include, but is not limited to, the following delivery methods: topical, oral, parenteral, subcutaneous, transdermal, transbuccal, intravascular (e.g., intravenous or intra-arterial), intramuscular, subcutaneous, intranasal, and intra-ocular administration. The term “administration” is inclusive of a self-administration and administration by another individual, such as by personnel of a health care provider. Administration can be local at a particular anatomical site, such as a site of infection or flare up, or systemic. Administration of steroid therapy and non-steroid therapy can be continuous or at distinct levels as can be readily determined by a person skilled in the art. In the context of administering a test steroid to a cell or sample of cells in vitro, administering means bringing the test steroid and cell(s) into contact with one another by any method.

As used herein, the term “biomarker” of the invention refers to one, two, three, four, five, or all six of MCAM, WFDC1, IGF1, IGFBP2, EYA4, and RBM24, and is inclusive of the polypeptide product (e.g., full-length protein or protein fragment) or nucleic acids (DNA or RNA) encoding the polypeptide product. The expression levels of one or more of the biomarkers may be determined at the mRNA or protein level.

As used herein, the terms “comprises,” “comprising,” “includes,” “including,” “has,” “having,” “contains,” or “containing,” or any other variation thereof, are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “contacting” in the context of determining the expression level of a gene in a sample means bringing the sample containing the nucleic acids and/or polypeptides in contact with the capture probe, or vice-versa, or any other manner of causing the capture probe and the sample to come into contact.

As used herein, the term “excipient” refers to an inert substance added to a pharmaceutical composition to further facilitate administration of an active ingredient. Examples of excipients include, but are not limited to, calcium carbonate, calcium phosphate, various sugars and types of starch, cellulose derivatives, gelatin, vegetable oils, and polyethylene glycols.

As used herein, the term “responsive” or “clinically responsive” in the context of steroid therapy refers to a favorable phenotype in an individual indicative of therapeutic efficacy. Responsiveness can be determined based in part or wholly on the expression level of one or more biomarkers used in the invention.

As used herein, the terms “subject”, “patient”, and “individual” refer to a human or non-human animal. Typically, the animal is a mammal. An individual also refers to for example, primates (e.g., humans), cows, sheep, goats, horses, dogs, cats, rabbits, rats, mice, fish, birds and the like. In certain embodiments, the individual is a primate. In yet other embodiments, the individual is a human. The individual may be any age, gender, or race.

As used herein, the term “steroid” includes steroids and steroid derivatives. In some embodiments, the steroid is a corticosteroid. In some embodiments, the steroid is a prodrug. The steroid may be a singular steroid or a combination of two or more steroids that utilize the same or different cellular pathways. In some embodiments, the steroid is a biologically active organic compound having a core structure composed of four rings. In some embodiments, the steroid has a typical steroid core structure composed of seventeen carbon atoms, bonded in four fused rings: three six-member cyclohexane rings and one five-member cyclopentane ring. This structure encompasses many steroids that vary by the functional groups attached to this four-ring core and by the oxidation state of the rings.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the subject. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to prophylaxis (preventing or delaying the onset, or development, or recurrence, or progression, of the disease or disorder). Thus, in some embodiments, the subject has the condition at the time the steroid therapy or non-steroid therapy is administered, such that the therapy would be administered to treat an existing condition. In other embodiments, the subject does not have the condition at the time the steroid therapy or non-steroid therapy is administered, such that the therapy would be administered to prevent or delay onset or recurrence of the condition (prophylaxis).

### ASPECTS

The present disclosure can be described in accordance with the following numbered Aspects, which should not be confused with the claims.

Aspect 1. A method for determining whether or not an individual will respond to a steroid therapy, the method comprising determining the expression level of one or more genes selected from among MCAM (SEQ ID NO. 7), WFDC1 (SEQ ID NO. 8), IGF1 (SEQ ID NO. 9), IGFBP2 (SEQ ID NO. 10), EYA4 (SEQ ID NO. 11), and RBM24 (SEQ ID NO. 12) in a sample obtained from the individual,

wherein a higher level of expression of one or both of IGF1 (SEQ ID NO. 9) and EYA4 (SEQ ID NO. 11), or a lower level of expression of one or more of MCAM (SEQ ID NO. 7), WFDC1 (SEQ ID NO. 8), IGFBP2 (SEQ ID NO. 10), and RBM24 (SEQ ID NO. 12), relative to a reference expression level that is representative of the level of expression of the one or more genes in an individual who is steroid resistant, indicates that the individual is steroid sensitive and therefore will be responsive to steroid therapy, and

wherein a lower level of expression of one or both of IGF1 (SEQ ID NO. 9) and EYA4 (SEQ ID NO. 11), or a higher level of expression of one or more of MCAM (SEQ ID NO. 7), WFDC1 (SEQ ID NO. 8), IGFBP2 (SEQ ID NO. 10), and RBM24 (SEQ ID NO. 12), relative to a reference expression level that is representative of the level of expression of the one or more genes in an individual who is steroid sensitive, indicates that the individual is steroid resistant and therefore will be non-responsive to steroid therapy.

Aspect 2. The method of aspect 1, wherein the expression level of two, three, four, five, or all six of the genes is determined.

Aspect 3. The method of aspect 1, wherein the expression level of the one or more genes is the level of mRNA.

Aspect 4. The method of aspect 1, wherein the expression level of the one or more genes is the level of protein.

Aspect 5. The method of aspect 1, wherein the expression level of the one or more genes is determined using a nucleic acid measurement technique comprising one or more of: polymerase chain reaction (PCR, such as reverse transcription quantitative PCR (RT-qPCR) and digital PCR), microarray analysis, whole transcriptome shotgun sequencing (RNA-seq), and direct multiplexed gene expression analysis; or wherein the expression level of the one or more genes is determined using a protein measurement technique comprising one or more of: a spectrometry method (e.g., high-performance liquid chromatography (HPLC), and liquid chromatography-mass spectrometry) or an immuno-based method (e.g., enzyme-linked immunosorbent assay (ELISA), protein immunoprecipitation, immunoelectrophoresis, Western blot, and protein immunostaining).

Aspect 6. The method of aspect 1, wherein the expression level of the one or more genes is determined using RT-PCR or a lateral flow-based rapid test.

Aspect 7. The method of aspect 1, wherein the reference expression level comprises a gene expression level for each gene for which the expression level is determined, wherein the reference expression level for each gene is representative of the median amount of gene expression product of the gene in a group of individuals.

Aspect 8. The method of aspect 1, wherein the sample is a body fluid, such as blood (whole blood, plasma, or serum), saliva, or urine.

Aspect 9. The method of aspect 1, wherein the sample is a cell sample comprising one or more cell types selected from among fibroblasts, epithelial cells, keratinocytes, and melanocytes.

Aspect 10. The method of aspect 1, wherein the individual has a condition, for which a steroid is a treatment, at the time the sample is obtained from the individual.

Aspect 11. The method of aspect 1, wherein the individual does not have a condition, for which a steroid is a treatment option, at the time the sample is obtained from the individual.

Aspect 12. The method of aspect 1, wherein the sample is a cell sample, and wherein cells of the cell sample comprise normal cells.

Aspect 13. The method of aspect 1, wherein the sample is a cell sample, wherein the cells of the cell sample comprise abnormal or diseased cells.

Aspect 14. The method of aspect 1, wherein the sample is a cell sample, and wherein the cell sample comprises cells of a keloid.

Aspect 15. The method of aspect 1, wherein the sample is a cell sample, and wherein the cell sample comprises buccal cells, or cells of a surgical biopsy.

Aspect 16. The method of aspect 1, wherein the steroid comprises a corticosteroid.

Aspect 17. The method of aspect 10, wherein the condition is an immune disorder, inflammatory disorder, hyper-proliferative disorder, or dermatological disorder.

Aspect 18. A method for selecting a therapy for an individual having a condition, comprising:

receiving results of the method of any one of aspects 1 to 17; and

selecting a steroid therapy for the individual if the individual is steroid sensitive, or withholding steroid therapy from the individual and optionally selecting a non-steroid therapy for the individual if the individual is steroid resistant.

Aspect 19. The method of aspect 18, wherein the condition is an immune disorder, inflammatory disorder, hyper-proliferative disorder, or dermatological disorder.

Aspect 20. A method for treating a condition in an individual who is classified as having a steroid sensitive phenotype (steroid responsive phenotype) or a steroid resistant phenotype, the method comprising:
(a) identifying the steroid sensitive phenotype or steroid resistant phenotype for the individual, wherein said identifying relies on a determination of the expression level of one or more genes selected from among MCAM (SEQ ID NO. 7), WFDC1 (SEQ ID NO. 8), IGF1 (SEQ ID NO. 9), IGFBP2 (SEQ ID NO. 10), EYA4 (SEQ ID NO. 11), and RBM24 (SEQ ID NO. 12) in a sample obtained from the individual,
   wherein a higher level of expression of one or both of IGF1 (SEQ ID NO. 9) and EYA4 (SEQ ID NO. 11), or a lower level of expression of one or more of MCAM (SEQ ID NO. 7), WFDC1 (SEQ ID NO. 8), IGFBP2 (SEQ ID NO. 10), and RBM24 (SEQ ID NO. 12), relative to a reference expression level that is representative of the level of expression of the one or more genes in an individual who has a steroid resistant phenotype, indicates that the individual has a steroid sensitive phenotype, and
   wherein a lower level of expression of one or both of IGF1 (SEQ ID NO. 9) and EYA4 (SEQ ID NO. 11), or a higher level of expression of one or more of MCAM (SEQ ID NO. 7), WFDC1 (SEQ ID NO. 8), IGFBP2 (SEQ ID NO. 10), and RBM24 (SEQ ID NO. 12), relative to a reference expression level that is representative of the level of expression of the one or more genes in an individual who has a steroid sensitive phenotype, indicates that the individual has a steroid resistant phenotype; and
(b) treating the individual classified as having a steroid sensitive phenotype with a steroid that is suitable for the condition, or withholding steroid treatment from the individual classified as having steroid resistant phenotype, and optionally treating the individual classified as having a steroid resistant phenotype with a non-steroidal treatment (e.g., a non-steroid drug) that is suitable for treatment of the condition.

Aspect 21. The method of aspect 20, wherein the condition is an immune disorder, inflammatory disorder, hyper-proliferative disorder, or dermatological disorder.

Aspect 22. A method for determining the expression level of one, two, three, four, five, or all six genes selected from among MCAM (SEQ ID NO. 7), WFDC1 (SEQ ID NO. 8), IGF1 (SEQ ID NO. 9), IGFBP2 (SEQ ID NO. 10), EYA4 (SEQ ID NO. 11), and RBM24 (SEQ ID NO. 12) in a sample obtained from an individual.

Aspect 23. The method of aspect 22, wherein the expression level of two, three, four, five, or all six of the genes is determined.

24. The method of aspect 22 or 23, wherein the expression level of the one or more genes is the level of mRNA.

Aspect 25. The method of aspect 22 or 23, wherein the expression level of the one or more genes is the level of protein.

Aspect 26. The method of any preceding aspect, wherein the sample is a cell sample comprising one or more cell types selected from among fibroblasts, epithelial cells, keratinocytes, and melanocytes.

Aspect 27. The method of any one of aspects 22 to 25, wherein the sample is a body fluid such as such as blood (whole blood, plasma, or serum), saliva, or urine.

Aspect 28. The method of any preceding aspect, wherein the sample includes cells that comprise or consist of normal cells.

Aspect 29. The method of any preceding aspect, wherein sample includes cells that comprise or consist of abnormal or diseased cells.

Aspect 30. The method of any preceding aspect, wherein the cell sample comprises cells of a keloid.

Aspect 31. The method of any one of aspects 22 to 26, wherein the cell sample comprises buccal cells, or cells of a surgical biopsy.

Aspect 32. The method of any preceding aspect, wherein the individual has an immune disorder, inflammatory disorder, hyper-proliferative disorder, or dermatological disorder.

Aspect 33. A detection device comprising one or more capture probes associated with a substrate, wherein the one or more capture probes hybridize with target nucleic acids encoding one or more of MCAM (SEQ ID NO. 7), WFDC1 (SEQ ID NO. 8), IGF1 (SEQ ID NO. 9), IGFBP2 (SEQ ID NO. 10), EYA4 (SEQ ID NO. 11), and RBM24 (SEQ ID NO. 12), or wherein the one or more capture probes have an affinity for target polypeptides of one or more of MCAM (SEQ ID NO. 1), WFDC1 (SEQ ID NO. 2), IGF1 (SEQ ID NO. 3), IGFBP2 (SEQ ID NO. 4), EYA4 (SEQ ID NO. 5), and RBM24 (SEQ ID NO. 6), and wherein the detection device includes capture probes that hybridize with no more than 500 total nucleic acid targets or that have affinity for no more than 500 total target polypeptides.

Aspect 34. The detection probe of aspect 33, wherein the device includes capture probes that hybridize with target nucleic acids encoding two, three, four, five, or all six of MCAM (SEQ ID NO. 7), WFDC1 (SEQ ID NO. 8), IGF1 (SEQ ID NO. 9), IGFBP2 (SEQ ID NO. 10), EYA4 (SEQ ID NO. 11), and RBM24 (SEQ ID NO. 12), or includes capture probes that have affinity for target polypeptides of two, three, four, five, or all six of MCAM (SEQ ID NO. 1), WFDC1 (SEQ ID NO. 2), IGF1 (SEQ ID NO. 3), IGFBP2 (SEQ ID NO. 4), EYA4 (SEQ ID NO. 5), and RBM24 (SEQ ID NO. 6).

Aspect 35. The detection probe of aspect 33, wherein the one or more capture probes that hybridize with target nucleic acids comprise oligonucleotide probes that hybridize with messenger RNA (mRNA) encoding one or more of MCAM (SEQ ID NO. 7), WFDC1 (SEQ ID NO. 8), IGF1 (SEQ ID NO. 9), IGFBP2 (SEQ ID NO. 10), EYA4 (SEQ ID NO. 11), and. RBM24 (SEQ ID NO. 12).

Aspect 36. The detection probe of aspect 33, wherein the one or more capture probes that have affinity for target polypeptides of one or more of MCAM (SEQ ID NO. 1), WFDC1 (SEQ ID NO. 2), IGF1 (SEQ ID NO. 3), IGFBP2 (SEQ ID NO. 4), EYA4 (SEQ ID NO. 5), and RBM24 (SEQ ID NO. 6) comprise antibodies or antigenic-binding fragments of antibodies.

Aspect 37. The detection probe of aspect 33, wherein the detection device includes capture probes that hybridize with no more than 50 total nucleic acid targets or that have affinity for no more than 50 total target polypeptides.

### MATERIALS AND METHODS

Culture of keloid-derived primary fibroblasts. Surgically excised keloid and normal skin explants were obtained from patients under Florida State University institutional review board guidelines (Approved by the Human Subjects Committee (HSC) of the Florida State University Institutional Review Board; HSC numbers: 2016.19175 and 2017.22173). The explants were cut into small pieces, digested with trypsin, and washed with and cultured in Dulbecco's Modified Eagle Medium containing 10% fetal bovine serum and antibiotic/antimycotic solution maintained at 37.8° C. (98.68° F.) and 5% carbon dioxide. Outgrowth of only primary dermal fibroblasts occurred under our culture conditions, and these were recovered by trypsinization for use in experiments at low passage. The keloid fibroblasts are morphologically identical to commercially obtained normal primary human dermal fibroblasts (HDF; Cell Applications, Inc., San Diego, CA) used as our normal controls. Normal and keloid fibroblasts that were used for these studies as well as the sequencing are listed in Table 2. Note: Hyperproliferative keloids are a type of steroid resistant keloid that grow faster in response to steroid treatment.

TABLE 2
Patient derived normal and keloid fibroblasts used in this study.

| Patient | Race | Location | Sex | Steroid sensitivity | Age | Comments |
|---|---|---|---|---|---|---|
| HDF1 | Caucasian | Ear | F | Sensitive | 48 | Normal skin |
| HDF2 | African American | Abdomen | F | Sensitive | 45 | Normal skin |
| HDF3 | Hispanic | Abdomen | F | Sensitive | 50 | Normal skin |
| HDF PA | African American | Ear | F | Sensitive | 21 | Normal skin |
| PA | African American | Ear | F | Sensitive | 21 | Keloid |
| P5 | Hispanic | Scalp | F | Sensitive | 23 | Keloid |
| P16 | African American | Ear | F | Hyperproliferative | 37 | Keloid |
| P17 | Caucasian | Ear | F | Sensitive | 23 | Keloid |
| P21 | African American | Ear | F | Hyperproliferative | 23 | Keloid |
| P24 | African American | Neck | M | Hyperproliferative | 51 | Keloid |

RNA extraction. Total RNA was isolated from keloid and normal fibroblasts by TRIzol reagent (Life Technologies #15596-018) after being treated with either the vehicle Dimethyl sulfoxide (DMSO) or 10 μM of Triamcinolone Acetonide (TA) (Sigma-Aldrich) for 96 hrs. Details of the samples used for RNA-seq are shown in Table 2. The quality and quantity of each RNA sample was measured by Tapestation (Agilent). 500 ng and 50 ng of total RNAs were used for library preparation for RNA-seq and miRNA-seq respectively.

RNA-seq. NEBNEXT ULTRA™ Directional RNA Library Prep Kit for Illumina (NEB, E7420) and NEBNEXT rRNA Depletion Kit (E6310) were used for library preparation. The RNA-seq libraries were sequenced on a NovaSeq 6000 sequencer (Illumina) to obtain 2×100 or 2×150-base reads. The raw data were trimmed with TrimGalore and Cutadapt. FASTQC was used to filter out reads of low to moderate quality. Paired-end reads were mapped to the GRCh38 reference genome using Salmon (28). The average mapped reads were 323M reads per sample and average mapped rate was 94.8%. The inventors used edgeR (29) and 3D RNA-seq (30) R package to identify differentially expressed genes (DEGs) at a 5% false discovery rate (FDR) (Padj≤0.05) using the Benjamini-Hochberg procedure to adjust P values with the log2 fold change (log2FC) larger than 1.5 (upregulated) or smaller than 1.5 (downregulated). Batch effects of biological replicates were removed from the data using the RUVSeq method (27). Annotation of the genes and pathways was provided by the KEGG database and GO annotations.

Quantitative reverse transcription PCR (qRT-PCR). For mRNA quantification, RNA was reverse transcribed into cDNA with an Omniscript RT kit (205113, Qiagen) following the manufacturer's instructions. qPCR was performed using a PerfeCTa SYBR Green SuperMix (95054-500, Quanta Bio). For miRNA quantification, RNA was reverse-transcribed, followed by qRT-PCR with MIR-X™ miRNA qRT-PCR SYBR kit (638315, 638316, Takara Bio). Primer details are shown in Table 3. The relative expression of mRNA was calculated using the 2—ΔΔCq method.

TABLE 3
Primers used for this study

| Targets | Forward Primer | Reverse Primer |
|---|---|---|
| MCAM | ATC GCT GCT GAG TGA ACC | CTA CTC TCT GCC TCA CAG |
| | ACA G | GTC A |
| | (SEQ ID NO: 13) | (SEQ ID NO: 14) |
| WFDC1 | AAC CTC GAT GGC TTG GTG | TCA GGA TGT GGC ACT CAT |
| | GCA A | AGC C |
| | (SEQ ID NO: 15) | (SEQ ID NO: 16) |
| IGFBP2 | AAG CAT GGC CTG TAC AAC CT | GGG TTC ACA CAC CAG CAC T |
| | (SEQ ID NO: 17) | (SEQ ID NO: 18) |
| RBM24 | CCA AGG ATC ATG CAA CCA G | GCA GGT ATC CCG AAA GGT |
| | (SEQ ID NO: 19) | CT |
| | | (SEQ ID NO: 20) |
| IGF1 | CTT CAG TTC GTG TGT GGA | CGC CCT CCG ACT GCT G |
| | GAC AG | (SEQ ID NO: 22) |
| | (SEQ ID NO: 21) | |
| EYA4 | GAA TAA CAC AGC CGA TGG | CCA GGT CAC TAT CAG GAG |
| | (SEQ ID NO: 23) | (SEQ ID NO: 24) |
| Human beta | CAC CAA CTG GGA CGA CAT | ACA GCC TGG ATA GCA ACG |
| Actin | (SEQ ID NO: 25) | (SEQ ID NO: 26) |
| Human HPRT | TAT GGC GAC CCG CAG CCC T | CAT CTC GAG CAA GAC GTT |
| | (SEQ ID NO: 27) | CAG |
| | | (SEQ ID NO: 28) |

Statistical analysis. The significance of the difference between the groups was analyzed statistically by Student's t-test with repeated measures when appropriate. Statistical analyses were performed using Excel and R software. The difference between the means for all conditions was considered statistically significant when a P value is less than 0.05.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Keloid and Normal Fibroblasts Exhibit Different Gene Expression Profiles

Since humans are the only species affected by keloids (21), in the absence of ideal animal model systems for the study of keloids, the inventors and others have previously shown that keloid dermal fibroblasts cultured in vitro from surgically excised keloids are a suitable model system to study keloids and test therapeutic strategies (14). The inventors applied RNA-seq to systematically study the transcriptomes of normal primary Human Dermal Fibroblasts (HDF) as well as keloid dermal fibroblasts. The inventors isolated total RNAs from 4 normal and 6 keloid fibroblast samples from patients of different races (Table 2) following treatment with or without 10 μM of the steroid triamcinolone acetonide (TA) for 4 days. Sequencing libraries were prepared following ribosomal RNA (rRNA) depletion and the samples were sequenced on an Illumina platform. The sequencing details and the data analysis pipeline is described in the Methods section.

The identification of genes in the inventors' RNA-seq data that had been previously implicated in keloid pathogenesis serves to bolster confidence in their RNA-sell data. Not surprisingly, quantitative Reverse Transcriptase Polymerase Chain Reaction (qRT-PCR) has confirmed the expression changes observed in about half of all the differentially expressed genes (DEGs) that the inventors have tested so far. Several of the DEGs identified in keloid fibroblasts and the pathways in which they are involved were studied further and the results are discussed in Example 2, below.

### Example 2—Differentially Expressed Genes in Steroid Resistant Keloid Fibroblasts

Intralesional steroid injections have widely been used for the therapy of excessive scars since the mid-1960s (22). However, previous data from clinical studies (12, 13) and in vitro results (14) suggested that different individuals may show highly variable effects of steroid treatments (15, 16). To understand the underlying molecular mechanisms of differential steroid sensitivity in keloids, the inventors identified. DEGs in steroid sensitive and resistant keloid fibroblasts following steroid treatment. A total of 526 genes are upregulated and 521 genes were downregulated in steroid sensitive keloid fibroblasts after TA treatment (FIG. 1A), while 131 genes are upregulated and 106 genes are downregulated in steroid resistant keloid fibroblasts following TA treatment (FIG. 113). Next, the inventors identified the top 20 upregulated and downregulated genes between steroid sensitive keloid fibroblasts and steroid resistant keloid fibroblasts (FIG. 1C). The inventors found that Eyes Absent Transcriptional Coactivator and Phosphatase 4 (EYA4) and Insulin-like growth factor 1 (IGF1) were downregulated in steroid resistant keloid fibroblasts compared to steroid sensitive keloid fibroblasts. Melanoma Cell Adhesion Molecule (MCAM), WAP Four-Disulfide Core Domain 1 (WFDC1), Insulin Like Growth Factor Binding Protein 2 (IGFBP2), and RNA Binding Motif Protein 24 (RBM24) are upregulated in steroid resistant keloid fibroblasts.

The inventors validated the differential expression of the genes between steroid sensitive and resistant keloid fibroblasts using GIRT-PCR (FIGS. 2A and 2B). Interestingly, it has been reported that the corticosteroid prednisolone increases serum IGF1 concentration and reduces IGFBP1 and IGFBP2 (23, 24). The inventors confirmed that IGF1 expression is downregulated, while IGFBP2 expression is upregulated in steroid resistant keloids compared to steroid sensitive keloids (FIGS. 2A and 2B). This strongly suggests that steroid resistant keloids may indeed be differentially regulated at the molecular level compared to steroid sensitive keloids. Additionally, the confirmed Dials may be used as molecular biomarkers in screening for steroid resistance in keloid therapy and beyond.

### Example 3—IGF1, IGFBP2, and MCAM are Present in Human Saliva

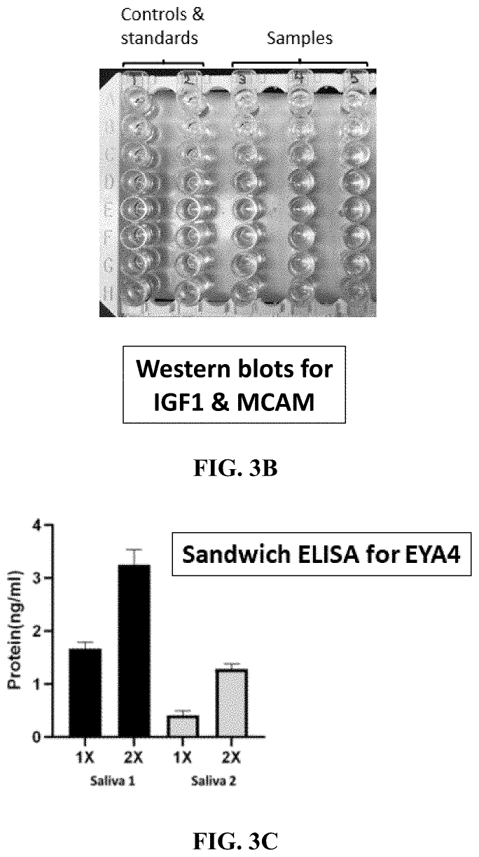

IGF1 IGFBP2, and MCAM proteins are present in human saliva and can be detected by Western blotting and ELISA. FIG. 3A shows the presence of MCAM and IGF1 in saliva samples from 3 different individuals using Western blot analysis. Tubulin has been previously reported to be present in saliva and its levels serve as a loading control. The use of twice as much saliva sample results in the detection of roughly twice as much IGF1 signal. FIG. 3B shows measurement of IGFBP2 levels in saliva and cell culture supernatants using highly specific sandwich ELISA kits. FIG. 3C shows measurement of EYA4 levels in saliva in ng/mL using MASA, Twice the amount of saliva results in roughly twice the signal. Colorimetrically detected IGFBP2 levels are provided in pg/mL of saliva or cell culture supernatant; numerical values are also provided in Table 4:

TABLE 4
IGFBP2 Levels in pg/mL using Sandwich ELISA

| Sample or Control | IGFBP2 Level in pg/mL |
|---|---|
| Saliva A | 1743 |
| Saliva B | 2276 |
| Saliva C | 2828 |
| PA Keloid cell culture media | 536 |
| P5 Keloid cell culture media | 364 |
| P17 keloid cell culture media | 688 |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES 1. P. D. Butler, M. T. Longaker, G. P. Yang, Current Progress in Keloid Research and Treatment. Journal of the American College of Surgeons 206, 731-741 (2008).

2. J. N. R. Bailey, A. E. Waite, W. J. Clayton, M. H. A. Rustin, Application of topical mitomycin C to the base of shave-removed keloid scars to prevent their recurrence. British Journal of Dermatology 156, 682-686 (2007).
3. G. G. Gauglitz, H. C. Korting, T. Pavicic, T. Ruzicka, M. G. Jeschke, Hypertrophic Scarring and Keloids: Pathomechanisms and Current and Emerging Treatment Strategies. Molecular Medicine 17, 113-125 (2011).
4. W. Mari et al., Novel Insights on Understanding of Keloid Scar: Article Review. Journal of the American College of Clinical Wound Specialists 7, 1-7 (2015).
5. X. Dong, S. Mao, H. Wen, Upregulation of proinflammatory genes in skin lesions may be the cause of keloid formation (Review). Biomedical Reports 1, 833-836 (2013).
6. R. A. F. Clark, Cutaneous tissue repair: Basic biologic considerations. I. Journal of the American Academy of Dermatology 13, 701-725 (1985).
7. B. Berman, H. C. Bieley, Adjunct therapies to surgical management of keloids. Dermatol Surg 22, 126-130 (1996).
8. J. Y. Jung, M. R. Roh, Y. S. Kwon, K. Y. Chung, Surgery and perioperative intralesional corticosteroid injection for treating earlobe keloids: a korean experience. Annals of dermatology 21, 221-225 (2009).
9. J. Kiil, Keloids treated with topical injections of triamcinolone acetonide (kenalog). Immediate and long-term results. Scand J Plast Reconstr Surg 11, 169-172 (1977).
10. J. A. Ledon, J. Savas, K. Franca, A. Chacon, K. Nouri, Intralesional treatment for keloids and hypertrophic scars: a review. Dermatol Surg 39, 1745-1757 (2013).
11. A. R. Shons, B. H. Press, The treatment of earlobe keloids by surgical excision and postoperative triamcinolone injection. Ann Plast Surg 10, 480-482 (1983).
12. A. T. Perdanasari et al., Recent Developments in the Use of Intralesional Injections Keloid Treatment. Archives of Plastic Surgery 41, 620 (2014).
13. C. Rogues, L. Téot, The Use of Corticosteroids to Treat Keloids: A Review. The International Journal of Lower Extremity Wounds 7, 137-145 (2008).
14. Y. Son et al., Treatment of keloids with a single dose of low-energy superficial X-ray radiation to prevent recurrence after surgical excision: An in vitro and in vivo study. Journal of the American Academy of Dermatology 83, 1304-1314 (2020).
15. M. Tirgan, Worsening of keloids after intralesional injections. Journal of the American Academy of Dermatology 68, AB68-AB68 (2013).
16. M. Tirgan, Intralesional triamcinolone acetonide in the treatment of keloid lesions—can the treatment be harmful to some patients? Results of an online survey. International Journal of Keloid Research 1, 21-28 (2017).
17. W.-S. Wu, F.-S. Wang, K. D. Yang, C.-C. Huang, Y.-R. Kuo, Dexamethasone Induction of Keloid Regression through Effective Suppression of VEGF Expression and Keloid Fibroblast Proliferation. Journal of Investigative Dermatology 126, 1264-1271 (2006).
18. A. Salem et al., Role of vascular endothelial growth factor in keloids: a clinicopathologic study. International Journal of Dermatology 48, 1071-1077 (2009).
19. F. B. Niessen, P. H. Spauwen, J. Schalkwijk, M. Kon, On the nature of hypertrophic scars and keloids: a review. Plastic And Reconstructive Surgery 104, 1435-1458 (1999).
20. J. C. Smith, B. E. Boone, S. R. Opalenik, S. M. Williams, S. B. Russell, Gene Profiling of Keloid Fibroblasts Shows Altered Expression in Multiple Fibrosis-Associated Pathways. Journal of Investigative Dermatology 128, 1298-1310 (2008).
21. J. Marttala, J. P. Andrews, J. Rosenbloom, J. Uitto, Keloids: Animal models and pathologic equivalents to study tissue fibrosis. Matrix Biol 51, 47-54 (2016).
22. M. Jalali, A. Bayat, Current use of steroids in management of abnormal raised skin scars. Surgeon 5, 175-180 (2007).
23. S. B. Wheatcroft, M. T. Kearney, IGF-dependent and IGF-independent actions of IGF-binding protein-1 and -2: implications for metabolic homeostasis. Trends in Endocrinology & Metabolism 20, 153-162 (2009).
24. N. Ramshanker et al., Effects of Prednisolone on Serum and Tissue Fluid IGF-I Receptor Activation and Post-Receptor Signaling in Humans. The Journal of Clinical Endocrinology & Metabolism 102, 4031-4040 (2017).
25. M. Tirgan. (Int J Keloid Res., 2017), vol. 1, pp. 21-28.
26. Y.-K. Liang et al., MCAM/CD146 promotes tamoxifen resistance in breast cancer cells through induction of epithelial—mesenchymal transition, decreased ERα expression and AKT activation. Cancer Letters 386, 65-76 (2017).
27. D. Risso, J. Ngai, T. P. Speed, S. Dudoit, Normalization of RNA-seq data using factor analysis of control genes or samples. Nature Biotechnology 32, 896-902 (2014).
28. Patio R, Duggal G, Love M I, Irizarry R A, Kingsford C. Salmon provides fast and bias-aware quantification of transcript expression. Nat Methods 14(4):417-419 (2017).
29. Robinson M D, McCarthy D J, Smyth G K. edgeR: a Bioconductor package for differential expression analysis of digital gene expression data. Bioinformatics 26(1):139-40 (2010).
30. Guo W, Tzioutziou N A, Stephen G, Milne I, Calixto CP, Waugh R, Brown J W S, Zhang R. 3D RNA-seq: a powerful and flexible tool for rapid and accurate differential expression and alternative splicing analysis of RNA-seq data for biologists. RNA Biol. 18(11):1574-1587 (2021).

We claim:

1. A method for determining whether or not an individual will respond to a steroid therapy, the method comprising determining an expression level of one or more genes selected from among MCAM (SEQ ID NO. 7), WFDC1 (SEQ ID NO. 8), IGF1 (SEQ ID NO. 9), IGFBP2 (SEQ ID NO. 10), EYA4 (SEQ ID NO. 11), and RBM24 (SEQ ID NO. 12) in a sample obtained from the individual,
wherein a higher level of expression of one or both of IGF1 (SEQ ID NO. 9) and EYA4 (SEQ ID NO. 11), or a lower level of expression of one or more of MCAM (SEQ ID NO. 7), WFDC1 (SEQ ID NO. 8), IGFBP2 (SEQ ID NO. 10), and RBM24 (SEQ ID NO. 12), relative to a reference expression level that is representative of the expression level of the one or more genes in an individual who is steroid resistant, indicates that the individual is steroid sensitive and therefore will be responsive to steroid therapy, and
wherein a lower level of expression of one or both of IGF1 (SEQ ID NO. 9) and EYA4 (SEQ ID NO. 11), or a higher level of expression of one or more of MCAM (SEQ ID NO. 7), WFDC1 (SEQ ID NO. 8), IGFBP2 (SEQ ID NO. 10), and RBM24 (SEQ ID NO. 12), relative to a reference expression level that is representative of the expression level of the one or more genes in an individual who is steroid sensitive, indicates that the individual is steroid resistant and therefore will be non-responsive to steroid therapy.

2. The method of claim 1, wherein the expression level of two, three, four, five, or all six of the genes is determined.

3. The method of claim 1, wherein the expression level of the one or more genes is determined as a level of mRNA.

4. The method of claim 1, wherein the expression level of the one or more genes is determined as a level of protein encoded by the one or more genes.

5. The method of claim 1, wherein the expression level of the one or more genes is determined using a nucleic acid measurement technique comprising one or more of: polymerase chain reaction, microarray analysis, whole transcriptome shotgun sequencing (RNA-seq), and direct multiplexed gene expression analysis; or
wherein the expression level of the one or more genes is determined using a protein measurement technique comprising one or more of: a spectrometry method or an immuno-based method.

6. The method of claim 1, wherein the expression level of the one or more genes is determined using RT-PCR or a lateral flow-based rapid test.

7. The method of claim 1, wherein the reference expression level comprises a gene expression level for each gene for which the expression level is determined, and wherein the reference expression level for each gene is representative of the median amount of gene expression product of the gene in a group of individuals.

8. The method of claim 1, wherein the sample comprises whole blood, plasma, serum, saliva, or urine.

9. The method of claim 1, wherein the sample is a cell sample comprising one or more cell types selected from among fibroblasts, epithelial cells, keratinocytes, and melanocytes.

10. The method of claim 1, wherein the individual has a condition for which steroid therapy is a treatment, at the time the sample is obtained from the individual.

11. The method of claim 1, wherein the sample is a cell sample, and wherein cells of the cell sample comprise normal cells.

12. The method of claim 1, wherein the sample is a cell sample, wherein the cells of the cell sample comprise abnormal or diseased cells.

13. The method of claim 1, wherein the sample is a cell sample, and wherein the cell sample comprises cells of a keloid, buccal cells, or cells from a surgical biopsy.

14. The method of claim 1, wherein the steroid comprises a corticosteroid.

15. The method of claim 14, wherein the corticosteroid comprises alclometasone, diproprionate, amcinonide, beclomethasone, betamethasone, betamethasone dipropionate, betamethasone valerate, budesonide, ciclesonide, clobetasol propionate, clobetasone butyrate, cortisone, cortisone acetate, desonide, dexamethasone, fluocinolone acetonide, fluocinonide, fluocortolone, fluprednidene acetate, flurohydrocortisone, halcinonide, halometasone, hydrocortisone, hydrocortisone aceponate, hydrocortisone acetate, hydrocortisone buteprate, hydrocortisone butyrate, hydrocortisone valerate, methylprednisolone, mometasone, mometasone furoate, prednicarabate, prednisolone, prednisone, tixocortol pivalate, triamcinolone acetonide, or any combination thereof.

16. The method of claim 10, wherein the condition is an immune disorder, an inflammatory disorder, a hyper-proliferative disorder, a dermatological disorder, or any combination thereof.

17. A method for selecting a therapy for an individual having a condition, comprising:
receiving results of the method of claim 1; and
selecting a steroid therapy for the individual if the individual is steroid sensitive, or withholding steroid therapy from the individual and optionally selecting a non-steroid therapy for the individual if the individual is steroid resistant.

18. The method of claim 17, wherein the condition is an immune disorder, inflammatory disorder, hyper-proliferative disorder, or dermatological disorder.

19. A method for treating a condition in an individual who is classified as having a steroid sensitive phenotype (steroid responsive phenotype) or a steroid resistant phenotype, the method comprising:
(a) identifying the steroid sensitive phenotype or steroid resistant phenotype for the individual, wherein said identifying relies on a determination of an expression level of one or more genes selected from among MCAM (SEQ ID NO. 7), WFDC1 (SEQ ID NO. 8), IGF1 (SEQ ID NO. 9), IGFBP2 (SEQ ID NO. 10), EYA4 (SEQ ID NO. 11), and RBM24 (SEQ ID NO. 12) in a sample obtained from the individual,
wherein a higher expression level of one or both of IGF1 (SEQ ID NO. 9) and EYA4 (SEQ ID NO. 11), or a lower expression level of one or more of MCAM (SEQ ID NO. 7), WFDC1 (SEQ ID NO. 8), IGFBP2 (SEQ ID NO. 10), and RBM24 (SEQ ID NO. 12), relative to a reference expression level that is representative of the expression level of the one or more genes in an individual who has a steroid resistant phenotype, indicates that the individual has a steroid sensitive phenotype, and
wherein a lower expression level of one or both of IGF1 (SEQ ID NO. 9) and EYA4 (SEQ ID NO. 11), or a higher expression level of one or more of MCAM (SEQ ID NO. 7), WFDC1 (SEQ ID NO. 8), IGFBP2 (SEQ ID NO. 10), and RBM24 (SEQ ID NO. 12), relative to a reference expression level that is representative of the expression level of the one or more genes in an individual who has a steroid sensitive phenotype, indicates that the individual has a steroid resistant phenotype; and
(b) treating the individual classified as having a steroid sensitive phenotype with a steroid that is suitable for the condition, or withholding steroid treatment from the individual classified as having steroid resistant phenotype, and optionally treating the individual classified as having a steroid resistant phenotype with a non-steroidal treatment that is suitable for treatment of the condition.

20. The method of claim 18, wherein the condition is an immune disorder, inflammatory disorder, hyper-proliferative disorder, or dermatological disorder.
